(19) United States
(12) Patent Application Publication
(10) Pub. No.: US 2019/0194315 A1
(43) Pub. Date: Jun. 27, 2019

(54) ANTIBODY DRUG CONJUGATES

(71) Applicants: Tinya ABRAMS, Acton, MA (US); Steven Bruce COHEN, San Diego, CA (US); Joseph Anthony D'Alessio, Boston, MA (US); Jason Damiano, Oakland, CA (US); Clemens Durr, Weil am Rhein (DE); Bernhard Hubert GEIERSTANGER, Solana Beach, CA (US); Qi-Ying HU, Needham, MA (US); Thomas HUBER, Basel (CH); Hidetomo IMASE, Somerville, MA (US); Yunho JIN, San Diego, CA (US); Daniel MENEZES, Berkeley, CA (US); Kathy MILLER, San Francisco, CA (US); Morvarid MOHSENI, Boston, MA (US); Weijia OU, San Diego, CA (US); Katherine RENDAHL, Berkeley, CA (US); Tetsuo UNO, San Diego, CA (US); Yongqin WAN, San Diego, CA (US); Xing WANG, San Diego, CA (US)

(72) Inventors: Tinya ABRAMS, Acton, MA (US); Steven Bruce COHEN, San Diego, CA (US); Joseph Anthony D'Alessio, Boston, MA (US); Jason Damiano, Oakland, CA (US); Clemens Durr, Weil am Rhein (DE); Bernhard Hubert GEIERSTANGER, Solana Beach, CA (US); Qi-Ying HU, Needham, MA (US); Thomas HUBER, Basel (CH); Hidetomo IMASE, Somerville, MA (US); Yunho JIN, San Diego, CA (US); Daniel MENEZES, Berkeley, CA (US); Kathy MILLER, San Francisco, CA (US); Morvarid MOHSENI, Boston, MA (US); Weijia OU, San Diego, CA (US); Katherine RENDAHL, Berkeley, CA (US); Tetsuo UNO, San Diego, CA (US); Yongqin WAN, San Diego, CA (US); Xing WANG, San Diego, CA (US)

(73) Assignee: Novartis AG, Basel (CH)

(21) Appl. No.: 15/737,042

(22) PCT Filed: Jun. 17, 2016
(86) PCT No.: PCT/IB2016/053595

§ 371 Date: Dec. 15, 2017

Related U.S. Application Data
(60) Provisional application No. 62/181,004, filed on Jun. 17, 2015.

(51) Int. Cl. C07K 16/28; A61K 47/65; A61K 47/68; A61P 35/00; A61K 38/07

(52) U.S. Cl. CPC C07K 16/28; A61K 47/65; A61K 47/6803; A61K 51/10; A61P 35/00; A61K 38/07; A61K 47/6849

(57) ABSTRACT

This application discloses anti-P-cadherin antibodies, antigen binding fragments thereof, and antibody drug conjugates of said antibodies or antigen binding fragments, particularly antibody drug conjugates comprising anti-P-cadherin antibodies conjugated to auristatin analogs. The invention also relates to methods of treating cancer using the antibody drug conjugates. Also disclosed herein are methods of making the antibodies, antigen binding fragments, and antibody drug conjugates, and methods of using the antibodies and antigen binding fragments as diagnostic reagents.

### FIELD OF THE INVENTION

The present invention generally relates to anti-P-cadherin antibodies, antibody fragments, antibody drug conjugates, and their uses for the treatment of cancer.

### BACKGROUND OF THE INVENTION

### P-Cadherin

Classical cadherins represent a family of cell adhesion molecules expressed in adherens-type junctions that mediate calcium-dependent cell-to-cell contacts. Placental cadherin (P-cadherin; also known as cadherin 3, type 1 or “CDH3”) has restricted expression in normal tissues but is known to be expressed in undifferentiated or under-differentiated cell types of several tissues, including the basal epithelial cells of the skin, esophagus, lung and oral cavity. (see, e.g., Albergaria et al., Int. J. Dev. Biol. 55:811-822 (2011)).

The structure of P-cadherin consists of 3 distinct domains: an extracellular domain (ECD) containing five cadherin repeats in tandem, a transmembrane domain, and an intracellular tail containing a catenin binding domain. The ECD mediates both cis- and trans interactions between multiple P-cadherin molecules, while the catenin binding domain links P-cadherin to proteins such as p120 catenin and consequently, cellular cytoskeletal elements. (see, e.g., Wu et al., PNAS 107:17592-7 (2010).

### P-Cadherin and Cancer

P-cadherin (also referred to as “Pcad” “PCad” “P-Cad, or CDH3), is also known to be overexpressed in a number of malignant tumors, including breast, gastric, endometrial, head and neck, and colorectal cancer, among others. The overexpression of P-cadherin in some breast, endometrial, ovarian, colorectal and bladder tumors has also been correlated with a worse prognosis compared to cases where P-cadherin expression levels are low or absent. In breast cancer, P-cadherin is frequently overexpressed in high grade invasive carcinomas and is a reliable marker of basal-like tumors. (see, e.g., Paredes et al., Br. Can. Res. 9:214-226 (2007); Sanders et al., Int. J. Can. 79:573-579 (1998); Albergaria et al., Int. J. Dev. Biol. 55:811-822 (2011); Sousa et al., Histol. Histopathol. 25:963-975 (2010))

In certain cancer types, such as breast and ovarian cancer, P-cadherin is known to promote tumor cell motility, invasiveness and metastasis. (see, e.g., Cheung et al., Oncogene 30:2964-74 (2011); Ribeiro et al, Oncogene 29 :392-402 (2010)).

Numerous cancer-relevant processes are known to promote the expression of P-cadherin mRNA and protein. Inactivation of the tumor suppressor BRCA1 through either mutation or loss of expression has been associated with increased P-cadherin expression in both breast cancer cell lines and patient specimens. The transcription factor C-EBPβ and the anti-estrogen ICI182780 (fulvestrant) are also known to disregulate P-cadherin expression and induce its upregulation in tumor cells, as is hypomethylation of the CDH3 promoter via other processes. In alveolar rhabdomyosarcoma, the chimeric oncogenic transcription factors PAX3-FOXOA1 and PAX7-FOXOA1 (resulting from translocations) directly induce P-cadherin expression, resulting in increased tumor aggressiveness. (see e.g. Albergaria et al., Int. J. Dev. Biol. 55:811-822 (2011); Thuault et al., Oncogene 15:1474-86 (2012); Ames et al., Clin. Can. Res. 11;4003-11 (2005); Gorski et al., Br. Can. Res. Treat. 122:721-31 (2010); Paredes et al., Clin. Can. Res. 11:5869-5877 (2005); Albergaria et al., Human Mol. Gen. 19:2554-2566 (2010).

### Antibody Drug Conjugates

Antibody drug conjugates (“ADCs”) have been used for the local delivery of cytotoxic agents in the treatment of cancer (see e.g., Lambert, Curr. Opinion In Pharmacology 5:543-549, 2005). ADCs allow targeted delivery of the drug moiety where maximum efficacy with minimal toxicity may be achieved. As more ADCs show promising clinical results, there is an increased need to develop new therapeutics for cancer therapy. Moreover, not all attempts to make therapeutically effective ADCs to known cancer targets have been successful. Examples of factors that can effect therapeutic effectiveness of ADCs include affinity, ability of an antibody to conjugate, the cleavability or stability of the linker; stability of the antibody-drug conjugate, the tendency of an antibody drug conjugate to aggregate, and the ratio of the drug/payload molecules that conjugate to each antibody (“DAR” or “drug antibody ratio”).

Aggregation and lack of stability can increase the possibility of adverse reactions to antibody drug conjugates in a clinical setting, reduce efficacy, as well as add to the cost of making ADCS.

Therefore there is a need for therapeutically effective ADC molecules.

### SUMMARY OF THE INVENTION

This application discloses anti-P-cadherin antibodies, antigen binding fragments thereof, and antibody drug conjugates of said antibodies or antigen binding fragments, particularly antibody drug conjugates comprising anti-P-cadherin antibodies conjugated to auristatin analogs.

In one embodiment, this application discloses an antibody that binds human P-cadherin selected from any one of the following:
a. An antibody or antigen binding fragment thereof comprising a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at positions 152 and 375, wherein said cysteine positions are numbered according to the EU system;
b. An antibody or antigen binding fragment thereof comprising a heavy chain variable region that comprises a VH CDR1 of SEQ ID NO: 21, a VH CDR2 of SEQ ID NO: 22, and a VH CDR3 of SEQ ID NO: 23, wherein the CDR is defined in accordance with the Kabat definition; and a light chain variable region that comprises a VL CDR1 of SEQ ID NO: 31, a VL CDR2 of SEQ ID NO: 32, and a VL CDR3 of SEQ ID NO: 33, wherein the CDR is defined in accordance with the Kabat definition, and a modified heavy chain constant region comprising cysteine at positions 152 and 375, wherein said cysteine positions are numbered according to the EU system;
c. An antibody or antigen binding fragment thereof comprising a heavy chain variable region that comprises a VH CDR1 of SEQ ID NO:41, a VH CDR2 of SEQ ID NO:42, and a VH CDR3 of SEQ ID NO:43, wherein the CDR is defined in accordance with the Kabat definition; and a light chain variable region that comprises a VL CDR1 of SEQ ID NO:51, a VL CDR2 of SEQ ID NO:52, and a VL CDR3 of SEQ ID NO:53, wherein the CDR is defined in accordance with the Kabat definition, and a modified heavy chain constant region comprising cysteine at positions 152 and 375, wherein said cysteine positions are numbered according to the EU system;
d. An antibody or antigen binding fragment thereof comprising a heavy chain variable region that comprises a VH CDR1 of SEQ ID NO:61, a VH CDR2 of SEQ ID NO:62, and a VH CDR3 of SEQ ID NO:63, wherein the CDR is defined in accordance with the Kabat definition; and a light chain variable region that comprises a VL CDR1 of SEQ ID NO:71, a VL CDR2 of SEQ ID NO:72, and a VL CDR3 of SEQ ID NO:73, wherein the CDR is defined in accordance with the Kabat definition, and a modified heavy chain constant region comprising cysteine at positions 152 and 375, wherein said cysteine positions are numbered according to the EU system;
e. An antibody or antigen binding fragment thereof comprising a heavy chain variable region that comprises a VH CDR1 of SEQ ID NO:81, a VH CDR2 of SEQ ID NO:82, and a VH CDR3 of SEQ ID NO:83, wherein the CDR is defined in accordance with the Kabat definition; and a light chain variable region that comprises a VL CDR1 of SEQ ID NO:91, a VL CDR2 of SEQ ID NO:92, and a VL CDR3 of SEQ ID NO:93, wherein the CDR is defined in accordance with the Kabat definition, and a modified heavy chain constant region comprising cysteine at positions 152 and 375, wherein said cysteine positions are numbered according to the EU system;
f. An antibody or antigen binding fragment thereof comprising a heavy chain variable region that comprises a VH CDR1 of SEQ ID NO:101, a VH CDR2 of SEQ ID NO:102, and a VH CDR3 of SEQ ID NO:103, wherein the CDR is defined in accordance with the Kabat definition; and a light chain variable region that comprises a VL CDR1 of SEQ ID NO:111, a VL CDR2 of SEQ ID NO:112, and a VL CDR3 of SEQ ID NO:113, wherein the CDR is defined in accordance with the Kabat definition, and a modified heavy chain constant region comprising cysteine at positions 152 and 375, wherein said cysteine positions are numbered according to the EU system;
g. An antibody or antigen binding fragment thereof comprising a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at positions 152 and 375, wherein said cysteine positions are numbered according to the EU system;
h. An antibody or antigen binding fragment thereof comprising a heavy chain variable region (VH) comprising the amino acid sequence of SEQ ID NO:27, and a light chain variable region (VL) comprising the amino acid sequence of SEQ ID NO:37, and a modified heavy chain constant region comprising cysteine at positions 152 and 375, wherein said cysteine positions are numbered according to the EU system;
i. An antibody or antigen binding fragment thereof comprising a heavy chain variable region (VH) comprising the amino acid sequence of SEQ ID NO:47, and a light chain variable region (VL) comprising the amino acid sequence of SEQ ID NO:57, and a modified heavy chain constant region comprising cysteine at positions 152 and 375, wherein said cysteine positions are numbered according to the EU system;
j. An antibody or antigen binding fragment thereof comprising a heavy chain variable region (VH) comprising the amino acid sequence of SEQ ID NO:67, and a light chain variable region (VL) comprising the amino acid sequence of SEQ ID NO:77, and a modified heavy chain constant region comprising cysteine at positions 152 and 375, wherein said cysteine positions are numbered according to the EU system;
k. An antibody or antigen binding fragment thereof comprising a heavy chain variable region (VH) comprising the amino acid sequence of SEQ ID NO:87, and a light chain variable region (VL) comprising the amino acid sequence of SEQ ID NO:97, and a modified heavy chain constant region comprising cysteine at positions 152 and 375, wherein said cysteine positions are numbered according to the EU system;
l. An antibody or antigen binding fragment thereof comprising a heavy chain variable region (VH) comprising the amino acid sequence of SEQ ID NO:107, and a light chain variable region (VL) comprising the amino acid sequence of SEQ ID NO:117, and a modified heavy chain constant region comprising cysteine at positions 152 and 375, wherein said cysteine positions are numbered according to the EU system;
m. An antibody or antigen binding fragment thereof comprising a heavy chain comprising the amino acid sequence of SEQ ID NO:130, and a light chain comprising the amino acid sequence of SEQ ID NO:19;
n. An antibody or antigen binding fragment thereof comprising a heavy chain comprising the amino acid sequence of SEQ ID NO:133, and a light chain comprising the amino acid sequence of SEQ ID NO:39;
o. An antibody or antigen binding fragment thereof comprising a heavy chain comprising the amino acid sequence of SEQ ID NO:136, and a light chain comprising the amino acid sequence of SEQ ID NO:59;
p. An antibody or antigen binding fragment thereof comprising a heavy chain comprising the amino acid sequence of SEQ ID NO:139, and a light chain comprising the amino acid sequence of SEQ ID NO:79;
q. An antibody or antigen binding fragment thereof comprising a heavy chain comprising the amino acid sequence of SEQ ID NO:142, and a light chain comprising the amino acid sequence of SEQ ID NO:99;
r. An antibody or antigen binding fragment thereof comprising a heavy chain comprising the amino acid sequence of SEQ ID NO:145, and a light chain comprising the amino acid sequence of SEQ ID NO:119;
s. An antibody or antigen binding fragment thereof comprising a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
t. An antibody or antigen binding fragment thereof comprising a heavy chain variable region that comprises a VH CDR1 of SEQ ID NO: 21, a VH CDR2 of SEQ ID NO: 22, and a VH CDR3 of SEQ ID NO: 23, wherein the CDR is defined in accordance with the Kabat definition; and a light chain variable region that comprises a VL CDR1 of SEQ ID NO: 31, a VL CDR2 of SEQ ID NO: 32, and a VL CDR3 of SEQ ID NO: 33, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
u. An antibody or antigen binding fragment thereof comprising a heavy chain variable region that comprises a VH CDR1 of SEQ ID NO:41, a VH CDR2 of SEQ ID NO:42, and a VH CDR3 of SEQ ID NO:43, wherein the CDR is defined in accordance with the Kabat definition; and a light chain variable region that comprises a VL CDR1 of SEQ ID NO:51, a VL CDR2 of SEQ ID NO:52, and a VL CDR3 of SEQ ID NO:53, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
v. An antibody or antigen binding fragment thereof comprising a heavy chain variable region that comprises a VH CDR1 of SEQ ID NO:61, a VH CDR2 of SEQ ID NO:62, and a VH CDR3 of SEQ ID NO:63, wherein the CDR is defined in accordance with the Kabat definition; and a light chain variable region that comprises a VL CDR1 of SEQ ID NO:71, a VL CDR2 of SEQ ID NO:72, and a VL CDR3 of SEQ ID NO:73, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
w. An antibody or antigen binding fragment thereof comprising a heavy chain variable region that comprises a VH CDR1 of SEQ ID NO:81, a VH CDR2 of SEQ ID NO:82, and a VH CDR3 of SEQ ID NO:83, wherein the CDR is defined in accordance with the Kabat definition; and a light chain variable region that comprises a VL CDR1 of SEQ ID NO:91, a VL CDR2 of SEQ ID NO:92, and a VL CDR3 of SEQ ID NO:93, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
x. An antibody or antigen binding fragment thereof comprising a heavy chain variable region that comprises a VH CDR1 of SEQ ID NO:101, a VH CDR2 of SEQ ID NO:102, and a VH CDR3 of SEQ ID NO:103, wherein the CDR is defined in accordance with the Kabat definition; and a light chain variable region that comprises a VL CDR1 of SEQ ID NO:111, a VL CDR2 of SEQ ID NO:112, and a VL CDR3 of SEQ ID NO:113, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
y. An antibody or antigen binding fragment thereof comprising a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at position 360, and a modified light chain constant region comprising cysteine at position 107, wherein said cysteine positions are numbered according to the EU system;
z. An antibody or antigen binding fragment thereof comprising a heavy chain variable region (VH) comprising the amino acid sequence of SEQ ID NO:27, and a light chain variable region (VL) comprising the amino acid sequence of SEQ ID NO:37, and a modified heavy chain constant region comprising cysteine at position 360, and a modified light chain constant region comprising cysteine at position 107, wherein said cysteine positions are numbered according to the EU system;
aa. An antibody or antigen binding fragment thereof comprising a heavy chain variable region (VH) comprising the amino acid sequence of SEQ ID NO:47, and a light chain variable region (VL) comprising the amino acid sequence of SEQ ID NO:57, and a modified heavy chain constant region comprising cysteine at position 360, and a modified light chain constant region comprising cysteine at position 107, wherein said cysteine positions are numbered according to the EU system;
bb. An antibody or antigen binding fragment thereof comprising a heavy chain variable region (VH) comprising the amino acid sequence of SEQ ID NO:67, and a light chain variable region (VL) comprising the amino acid sequence of SEQ ID NO:77, and a modified heavy chain constant region comprising cysteine at position 360, and a modified light chain constant region comprising cysteine at position 107, wherein said cysteine positions are numbered according to the EU system;
cc. An antibody or antigen binding fragment thereof comprising a heavy chain variable region (VH) comprising the amino acid sequence of SEQ ID NO:87, and a light chain variable region (VL) comprising the amino acid sequence of SEQ ID NO:97, and a modified heavy chain constant region comprising cysteine at position 360, and a modified light chain constant region comprising cysteine at position 107, wherein said cysteine positions are numbered according to the EU system;
dd. An antibody or antigen binding fragment thereof comprising a heavy chain variable region (VH) comprising the amino acid sequence of SEQ ID NO:107, and a light chain variable region (VL) comprising the amino acid sequence of SEQ ID NO:117and a modified heavy chain constant region comprising cysteine at position 360, and a modified light chain constant region comprising cysteine at position 107, wherein said cysteine positions are numbered according to the EU system;
ee. An antibody or antigen binding fragment thereof comprising a heavy chain comprising the amino acid sequence of SEQ ID NO:131, and a light chain comprising the amino acid sequence of SEQ ID NO:132;
ff. An antibody or antigen binding fragment thereof comprising a heavy chain comprising the amino acid sequence of SEQ ID NO:134, and a light chain comprising the amino acid sequence of SEQ ID NO:135;
gg. An antibody or antigen binding fragment thereof comprising a heavy chain comprising the amino acid sequence of SEQ ID NO:137, and a light chain comprising the amino acid sequence of SEQ ID NO:138;
hh. An antibody or antigen binding fragment thereof comprising a heavy chain comprising the amino acid sequence of SEQ ID NO:140, and a light chain comprising the amino acid sequence of SEQ ID NO:141;
ii. An antibody or antigen binding fragment thereof comprising a heavy chain comprising the amino acid sequence of SEQ ID NO:143, and a light chain comprising the amino acid sequence of SEQ ID NO:144; or
jj. An antibody or antigen binding fragment thereof comprising a heavy chain comprising the amino acid sequence of SEQ ID NO:146, and a light chain comprising the amino acid sequence of SEQ ID NO:147.

In another embodiment, this application discloses an antibody drug conjugate comprising a formula selected from:

(Formula A) or ((D)_{z})-L)_{y}-Ab (Formula B)

or a pharmaceutically acceptable salt thereof, wherein:
Ab is an antibody or antigen binding fragment thereof that binds human P-cadherin and is selected from any one of the following:
a. An antibody or antigen binding fragment thereof comprising a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition;
b. An antibody or antigen binding fragment thereof comprising a heavy chain variable region that comprises a VH CDR1 of SEQ ID NO: 21, a VH CDR2 of SEQ ID NO: 22, and a VH CDR3 of SEQ ID NO: 23, wherein the CDR is defined in accordance with the Kabat definition; and a light chain variable region that comprises a VL CDR1 of SEQ ID NO: 31, a VL CDR2 of SEQ ID NO: 32, and a VL CDR3 of SEQ ID NO: 33, wherein the CDR is defined in accordance with the Kabat definition;
c. An antibody or antigen binding fragment thereof comprising a heavy chain variable region that comprises a VH CDR1 of SEQ ID NO:41, a VH CDR2 of SEQ ID NO:42, and a VH CDR3 of SEQ ID NO:43, wherein the CDR is defined in accordance with the Kabat definition; and a light chain variable region that comprises a VL CDR1 of SEQ ID NO:51, a VL CDR2 of SEQ ID NO:52, and a VL CDR3 of SEQ ID NO:53, wherein the CDR is defined in accordance with the Kabat definition;
d. An antibody or antigen binding fragment thereof comprising a heavy chain variable region that comprises a VH CDR1 of SEQ ID NO:61, a VH CDR2 of SEQ ID NO:62, and a VH CDR3 of SEQ ID NO:63, wherein the CDR is defined in accordance with the Kabat definition; and a light chain variable region that comprises a VL CDR1 of SEQ ID NO:71, a VL CDR2 of SEQ ID NO:72, and a VL CDR3 of SEQ ID NO:73, wherein the CDR is defined in accordance with the Kabat definition;
e. An antibody or antigen binding fragment thereof comprising a heavy chain variable region that comprises a VH CDR1 of SEQ ID NO:81, a VH CDR2 of SEQ ID NO:82, and a VH CDR3 of SEQ ID NO:83, wherein the CDR is defined in accordance with the Kabat definition; and a light chain variable region that comprises a VL CDR1 of SEQ ID NO:91, a VL CDR2 of SEQ ID NO:92, and a VL CDR3 of SEQ ID NO:93, wherein the CDR is defined in accordance with the Kabat definition;
f. An antibody or antigen binding fragment thereof comprising a heavy chain variable region that comprises a VH CDR1 of SEQ ID NO:101, a VH CDR2 of SEQ ID NO:102, and a VH CDR3 of SEQ ID NO:103, wherein the CDR is defined in accordance with the Kabat definition; and a light chain variable region that comprises a VL CDR1 of SEQ ID NO:111, a VL CDR2 of SEQ ID NO:112, and a VL CDR3 of SEQ ID NO:113, wherein the CDR is defined in accordance with the Kabat definition;
g. An antibody or antigen binding fragment thereof comprising a heavy chain variable region (VH) comprising the amino acid sequence of SEQ ID NO:7, and a light chain variable region (VL) comprising the amino acid sequence of SEQ ID NO:17;
h. An antibody or antigen binding fragment thereof comprising a heavy chain variable region (VH) comprising the amino acid sequence of SEQ ID NO:27, and a light chain variable region (VL) comprising the amino acid sequence of SEQ ID NO:37;
i. An antibody or antigen binding fragment thereof comprising a heavy chain variable region (VH) comprising the amino acid sequence of SEQ ID NO:47, and a light chain variable region (VL) comprising the amino acid sequence of SEQ ID NO:57;
j. An antibody or antigen binding fragment thereof comprising a heavy chain variable region (VH) comprising the amino acid sequence of SEQ ID NO:67, and a light chain variable region (VL) comprising the amino acid sequence of SEQ ID NO:77;
k. An antibody or antigen binding fragment thereof comprising a heavy chain variable region (VH) comprising the amino acid sequence of SEQ ID NO:87, and a light chain variable region (VL) comprising the amino acid sequence of SEQ ID NO:97;
l. An antibody or antigen binding fragment thereof comprising a heavy chain variable region (VH) comprising the amino acid sequence of SEQ ID NO:107, and a light chain variable region (VL) comprising the amino acid sequence of SEQ ID NO:117,
m. An antibody or antigen binding fragment thereof comprising a heavy chain comprising the amino acid sequence of SEQ ID NO:9, and a light chain comprising the amino acid sequence of SEQ ID NO:19;
n. An antibody or antigen binding fragment thereof comprising a heavy chain comprising the amino acid sequence of SEQ ID NO:29, and a light chain comprising the amino acid sequence of SEQ ID NO:39;
o. An antibody or antigen binding fragment thereof comprising a heavy chain comprising the amino acid sequence of SEQ ID NO:49, and a light chain comprising the amino acid sequence of SEQ ID NO:59;
p. An antibody or antigen binding fragment thereof comprising a heavy chain comprising the amino acid sequence of SEQ ID NO:69, and a light chain comprising the amino acid sequence of SEQ ID NO:79;
q. An antibody or antigen binding fragment thereof comprising a heavy chain comprising the amino acid sequence of SEQ ID NO:89, and a light chain comprising the amino acid sequence of SEQ ID NO:99;
r. An antibody or antigen binding fragment thereof comprising a heavy chain comprising the amino acid sequence of SEQ ID NO:109, and a light chain comprising the amino acid sequence of SEQ ID NO:119;
s. An antibody or antigen binding fragment thereof selected from any one of the antibodies or antigen binding fragments thereof of claim 1;
t. An antibody or antigen binding fragment thereof that binds to human P-cadherin protein at one or more residues selected from the amino acids at positions 124, 125, 151, 153, 154, 155, 156, 159, 160, 161, 162, 163, 168, 170, 171, and 172 of SEQ ID NO:126;
u. An antibody or antigen binding fragment thereof that binds to human P-cadherin protein at the amino acids at positions 124, 125, 151, 153, 154, 155, 156, 159, 160, 161, 162, 163, 168, 170, 171, and 172 of SEQ ID NO:126;
v. An antibody or antigen binding fragment thereof comprising a heavy chain variable region that binds to human P-cadherin at one or more amino acid residues selected from positions 124, 151, 153-156, and 172 of SEQ ID NO:126;
w. An antibody or antigen binding fragment thereof comprising a heavy chain variable region binding paratope for human P-cadherin protein comprises one or more amino acid residues selected from positions 52, 54, 56, 60, 65, 105, or 107 of SEQ ID NO:128;
x. An antibody or antigen binding fragment thereof comprising a light chain variable region that binds to human P-cadherin at one or more amino acid residues selected from positions 124, 125, 155, 156, 159-163, 168, 170, and 171 of SEQ ID NO:126;
y. An antibody or antigen binding fragment thereof comprising a light chain variable region binding paratope for human P-cadherin protein comprises one or more amino acid residues selected from positions 1, 2, 27, 28, 30, 68, 92, 93, or 94 of SEQ ID NO:129;
z. An antibody or antigen binding fragment thereof comprising a heavy chain variable region that binds to human P-cadherin at one or more amino acid residues selected from positions 124, 151, 153-156, and 172 of SEQ ID NO:126; and the light chain variable region that binds to human P-cadherin at one or more amino acid residues selected from positions 124, 125, 155, 156, 159-163, 168, 170, and 171 of SEQ ID NO:126; or
aa. An antibody or antigen binding fragment thereof that binds to the same epitope of human P-cadherin as any of the antibodies a-z above, or competes with any one of the antibodies a-z above for binding to human P-cadherin;
z is an integer from 1 to 8;
y is an integer from 1 to 16;
L is a linker;
wherein when the antibody drug conjugate comprises Formula A, D is:

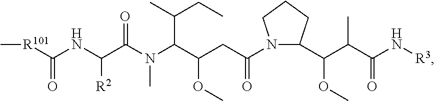

wherein
R¹⁰¹ is a 6 membered heterocycloalkyl divalent radical containing 1-2 N heteroatoms and a C₁-C₂alkylene bridge, wherein the 6 membered heterocycloalkyl divalent radical is C-linked to the

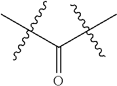 [SMILES: *C(*)=O]

group and is N-linked to L or is C-linked to L, and the 6 membered heterocycloalkyl divalent radical is unsubstituted or substituted with 1 to 3 substituents independently selected from R⁵ and R⁶;
or R¹⁰¹ is a 5-8 membered fused bicyclic heterocycloalkyl divalent radical containing 1-2 N heteroatoms, wherein the 5-8 membered fused bicyclic heterocycloalkyl divalent radical is C-linked to the

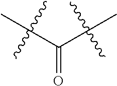 [SMILES: *C(*)=O]

group and is N-linked to L or is C-linked to L, and the 5-8 membered fused bicyclic heterocycloalkyl divalent radical is unsubstituted or substituted with 1 to 3 substituents independently selected from R⁵ and R⁶;
R² is —C₁-C₆alkyl;
R³ is

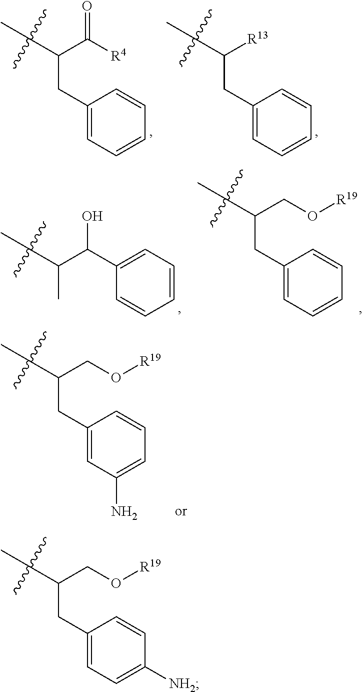

R⁴ is —OH, C₁-C₆alkoxy, —N(R¹⁴)₂, —R¹⁶, —NR¹²(CH₂)ₘN(R₁₄)₂, or —NR¹²(CH₂)ₘR¹⁶, —NHS(O)₂R₁₁ or

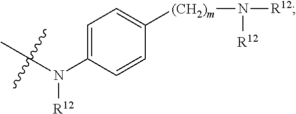

R⁵ is C₁-C₆alkyl, —C(═O)R¹¹, —(CH₂)ₘOH, —C(═O)(CH₂)ₘOH, —C(═O)((CH₂)ₘO)ₙR¹², —((CH₂)ₘO)ₙR¹², or C₁-C₆alkyl which is optionally substituted with —CN, —C(═O)NH₂ or 1 to 5 hydroxyl,
R⁶ is halo, oxo, OH, C₁-C₆alkyl, —N(R¹⁴)₂, —R¹⁶ and —NR¹²C(═O)R¹¹;
R¹¹ is C₁-C₆alkyl or C₁-C₆alkyl which is optionally substituted with 1 to 5 hydroxyl;
each R¹² is independently selected from H and C₁-C₆alkyl;
R¹³ is tetrazolyl, imidazolyl substituted with phenyl, oxadiazolyl substituted with phenyl, pyrazolyl, pyrimidinyl,

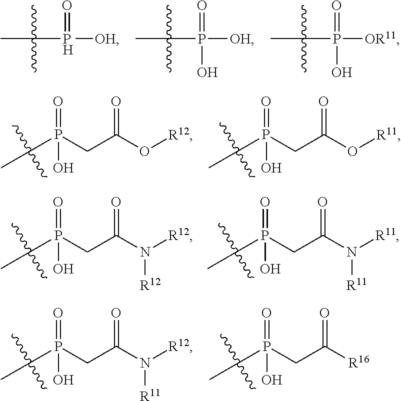

or —CH₂S(═O)₂NH₂;
each R¹⁴ is independently selected from H and C₁-C₆alkyl;
R¹⁶ is an N-linked 4-8 membered heterocycloalkyl containing 1-2 heteroatoms independently selected from N and O;
R¹⁹ is H or C₁-C₆alkyl;
each z is independently selected from 1, 2, 3, 4, 5, 6, 7, 8, 9 and 10,
and
each y is independently selected from 1, 2, 3, 4, 5, 6, 7, 8, 9, 10, 11, 12, 13, 14, 15, 16,17 and 18;
and wherein when the antibody drug conjugate comprises Formula B, D is

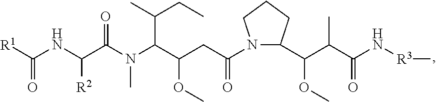

wherein
R¹ is a 6 membered heterocycloalkyl containing 1-2 N heteroatoms and a C₁-C₂alkylene bridge, wherein the 6 membered heterocycloalkyl is unsubstituted or substituted with 1 to 3 substituents independently selected from R⁵ and R⁶;
or R¹ is a 5-8 membered fused bicyclic heterocycloalkyl containing 1-2 N heteroatoms, wherein the 5-8 membered fused bicyclic heterocycloalkyl is unsubstituted or substituted with 1 to 3 substituents independently selected from R⁵ and R⁶;
R² is —C₁-C₆alkyl;
R³ is

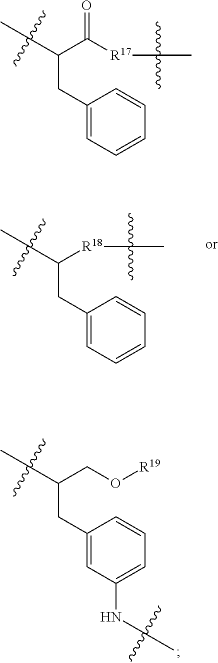

R⁵ is C₁-C₆alkyl, C₁-C₆alkyl which is optionally substituted with 1 to 5 hydroxyl, —C(═O)R¹¹, —(CH₂)ₘOH, —C(═O)(CH₂)ₘOH, —C(═O)((CH₂)ₘO)ₙR¹², or —((CH₂)ₘO)ₙR¹²;
R⁶ is halo, oxo, OH, C₁-C₆alkyl, —N(R¹⁴)₂, —R¹⁶ and —NR¹²C(═O)R¹¹;
R¹¹ is C₁-C₆alkyl or C₁-C₆alkyl which is optionally substituted with 1 to 5 hydroxyl;
each R¹² is independently selected from H and C₁-C₆alkyl;
each R¹⁴ is independently selected from H and C₁-C₆alkyl;
R¹⁶ is an N-linked 4-8 membered heterocycloalkyl containing 1-2 heteroatoms independently selected from N and O;
R¹⁷ is a bond, —NH—, —NHS(═O)₂—,

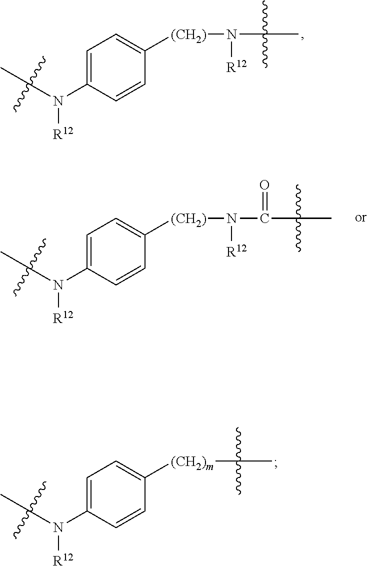

R¹⁸ is a bond,

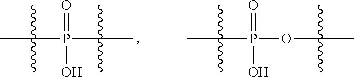 [SMILES: *P(*)(=O)O ; *OP(*)(=O)O]

or —CH₂S(═O)₂NH—;
each z is independently selected from 1, 2, 3, 4, 5, 6, 7, 8, 9 and 10,
and
each y is independently selected from 1, 2, 3, 4, 5, 6, 7, 8, 9, 10, 11, 12, 13, 14, 15, 16,17 and 18.

The Ab of the antibody drug conjugate is selected from any one of the antibodies or antigen binding fragments disclosed herein. In some embodiments, the antibody drug conjugates comprise an Ab that is conjugated to L via a thiol-maleimide linkage at the cysteine residues at positions 152 and 375 of the heavy chain constant region of the antibody, wherein said cysteine positions are numbered according to the EU system. In other embodiments, the antibody drug conjugate comprises an Ab that is conjugated to L via a thiol-maleimide linkage at the cysteine residue at position 360 of the heavy chain constant region of the antibody and position 107 of the light chain constant region, wherein said cysteine positions are numbered according to the EU system. 6. In yet other embodiments of the antibody drug conjugate, the antibody or antigen binding fragment thereof is conjugated to L via an oxime linkage at one or more interchain disulfide bridges of the antibody.

In some embodiments of this application, L is selected from -L₁L₂L₃L₄L₅L₆-, -L₆L₅L₄L₃L₂L₁-, -L₁L₂L₃L₄L₅-, -L₅L₄L₃L₂L₁-, -L₁L₂L₃L₄-, -L₄L₃L₂L₁-, -L₁L₂L₃-, -L₃-L₂-L₁-, -L₁-L₂-, -L₂-L₁- and -L₁; wherein L₂, L₃, L₄, L₅, and L₆ are each independently selected from a bond and L₁;
L₁ is selected from —(CH₂)ₘ—, —C(═O)(CH₂)ₘ—, —C(═O)X₁X₂C(═O)(CH₂)ₘ—, —C(═O)((CH₂)ₘO)ₙ(CH₂)ₘ—, —(CH₂)ₘS(═O)₂((CH₂)ₘO)ₙ(CH₂)ₘ—, —C(═O)(CH₂)ₘNR¹²(CH₂)ₘ—, —(CH₂)ₘC(═O)—, —((CH₂)ₘO)ₙ(CH₂)ₘ—, —(CH₂)ₘ(O(CH₂)ₘ)ₙ—, —(CH₂)ₘX₃((CH₂)ₘO)ₙ(CH₂)ₘ—, —C(═O)(CH₂)ₘX₃(CH₂)ₘ—, —C(═O)((CH₂)ₘO)ₙ(CH₂)ₘX₃(CH₂)ₘ—, —C(═O)(CH₂)ₘNR¹²C(═O)(CH₂)ₘ—, —NR¹²C(═O)(CH₂)ₘ—, —(CH₂)ₘC(═O)NR¹²—, —(CH₂)ₘNR¹²(CH₂)ₘ—, —(CH₂)ₘX₃(CH₂)ₘ—, —((CH₂)ₘO)ₙ(CH₂)ₘX₃(CH₂)ₘ—, —(CH₂)ₘX₃(CH₂)ₘ(O(CH₂)ₘ)ₙ—, —NR¹²(CH₂)ₘ—, —(CH₂)ₘNR¹²—, —S(═O)₂(CH₂)ₘ—, —C(═O)O—, —S—,

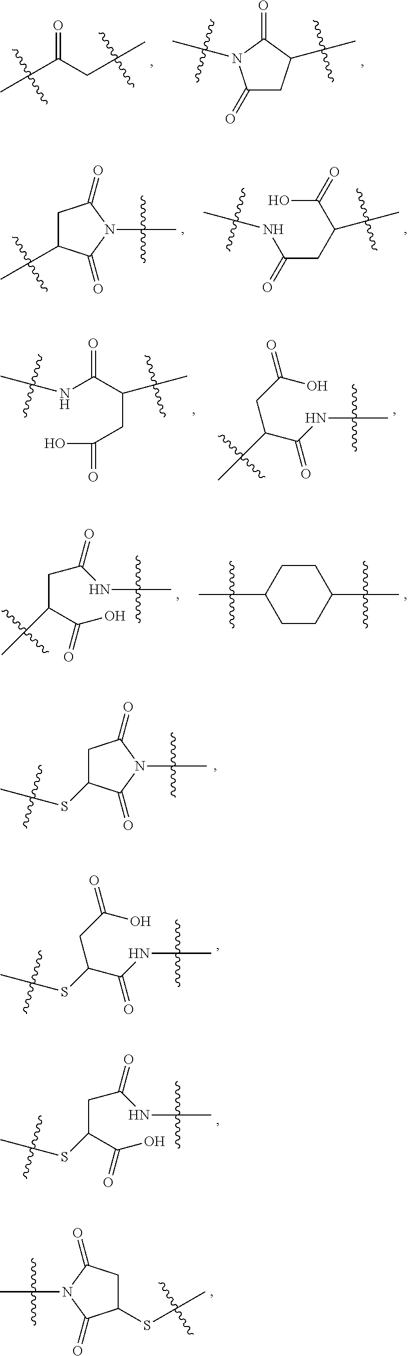

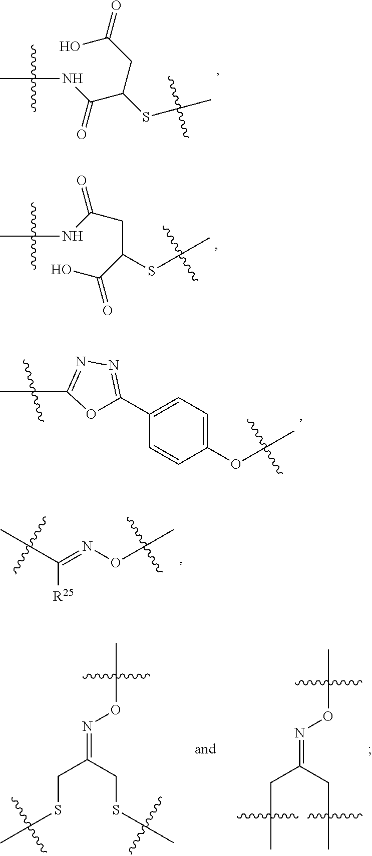

each R²⁵ is independently selected from H or C₁₋₄ alkyl;
X₁ is self immolative spacer selected from

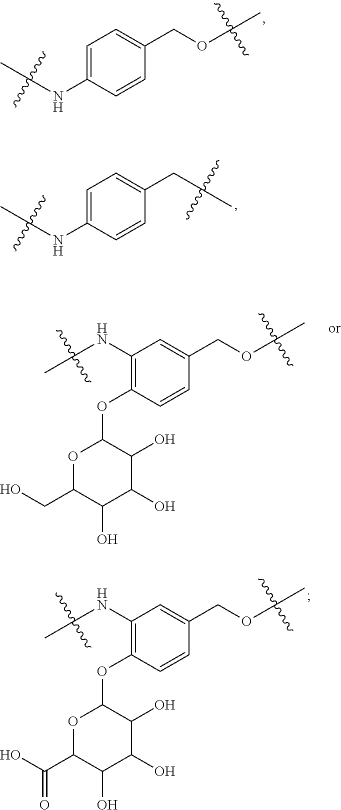 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is dipeptide selected from

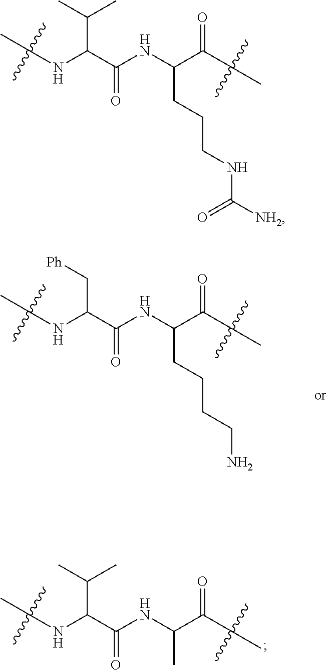 [SMILES: *NC(C(=O)NC(CCCNC(N)=O)C(*)=O)C(C)C ; *NC(Cc1ccccc1)C(=O)NC(CCCCN)C(*)=O ; "or" ; *NC(C(=O)NC(C)C(*)=O)C(C)C]

X₃ is

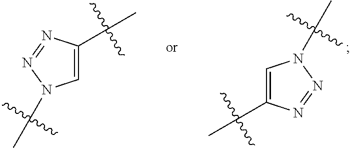 [SMILES: *c1cn(*)nn1 ; *c1cn(*)nn1]

each m is independently selected from 1, 2, 3, 4, 5, 6, 7, 8, 9 and 10, and
each n is independently selected from 1, 2, 3, 4, 5, 6, 7, 8, 9, 10, 11, 12, 13, 14, 15, 16,17 and 18.

In some embodiments of this application, D is selected from any one of the following structures and is conjugated to Ab via L to form the antibody drug conjugate of Formula A or Formula B:

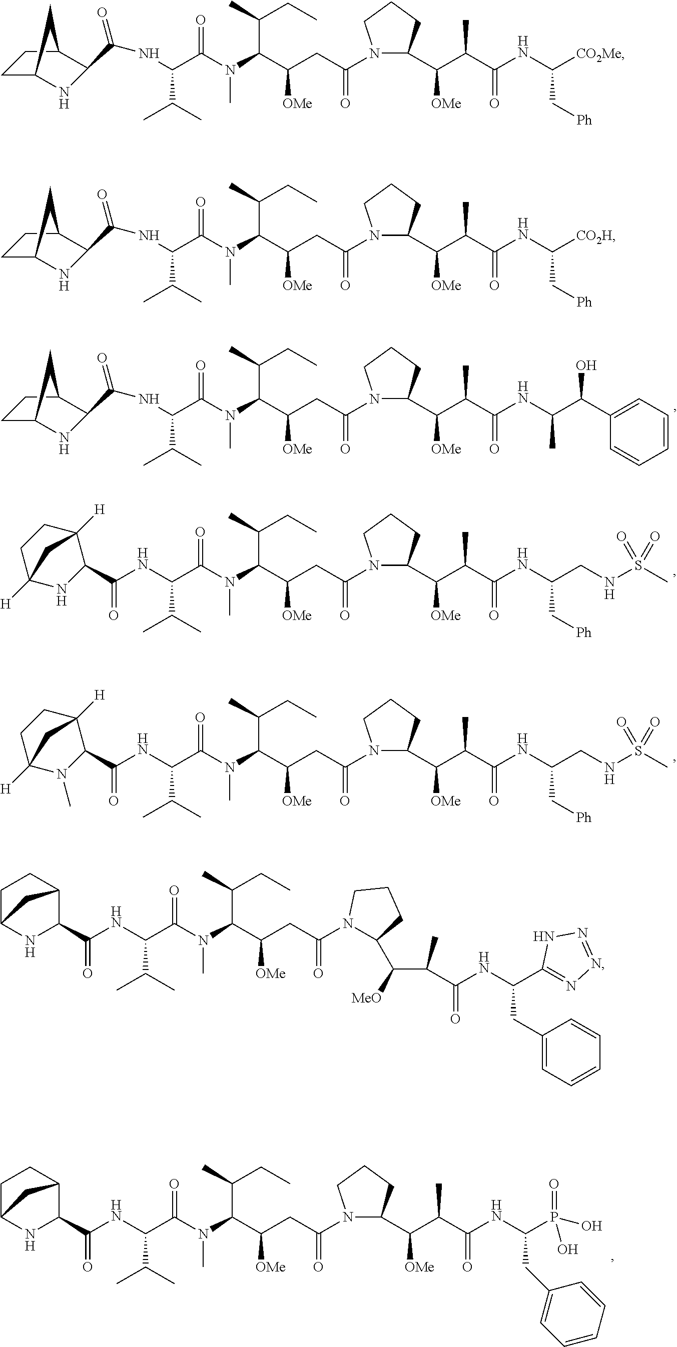

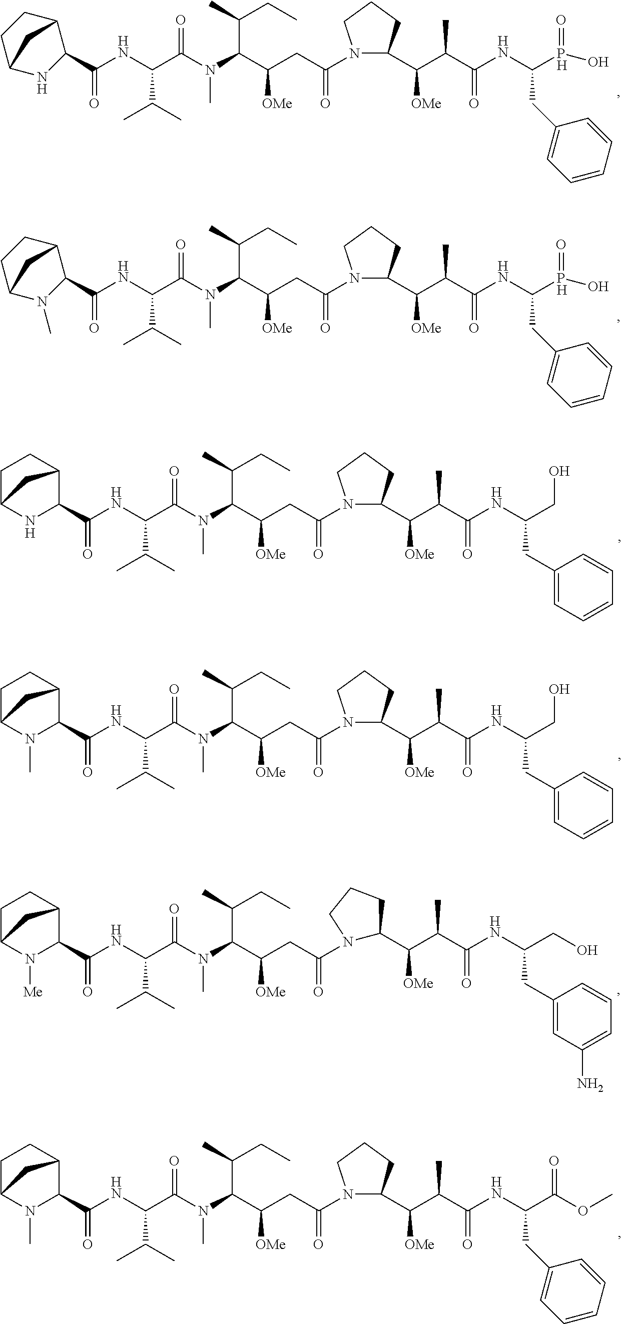

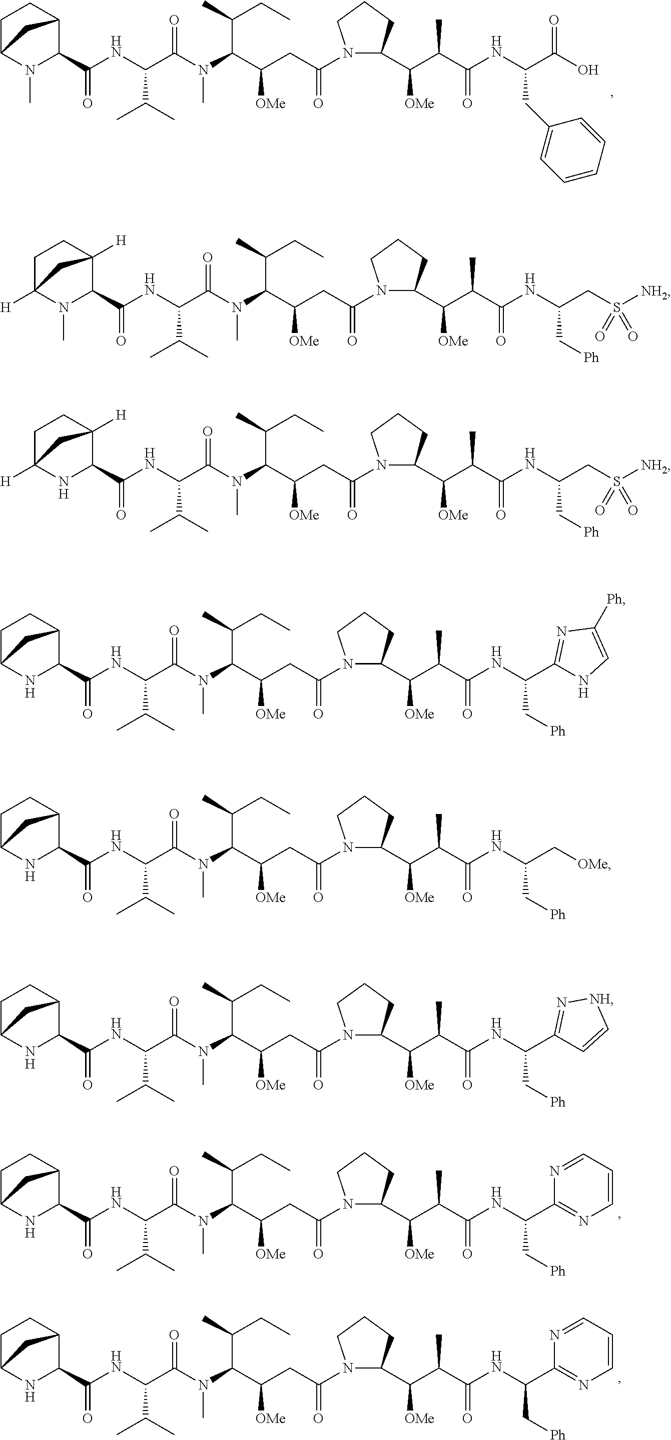

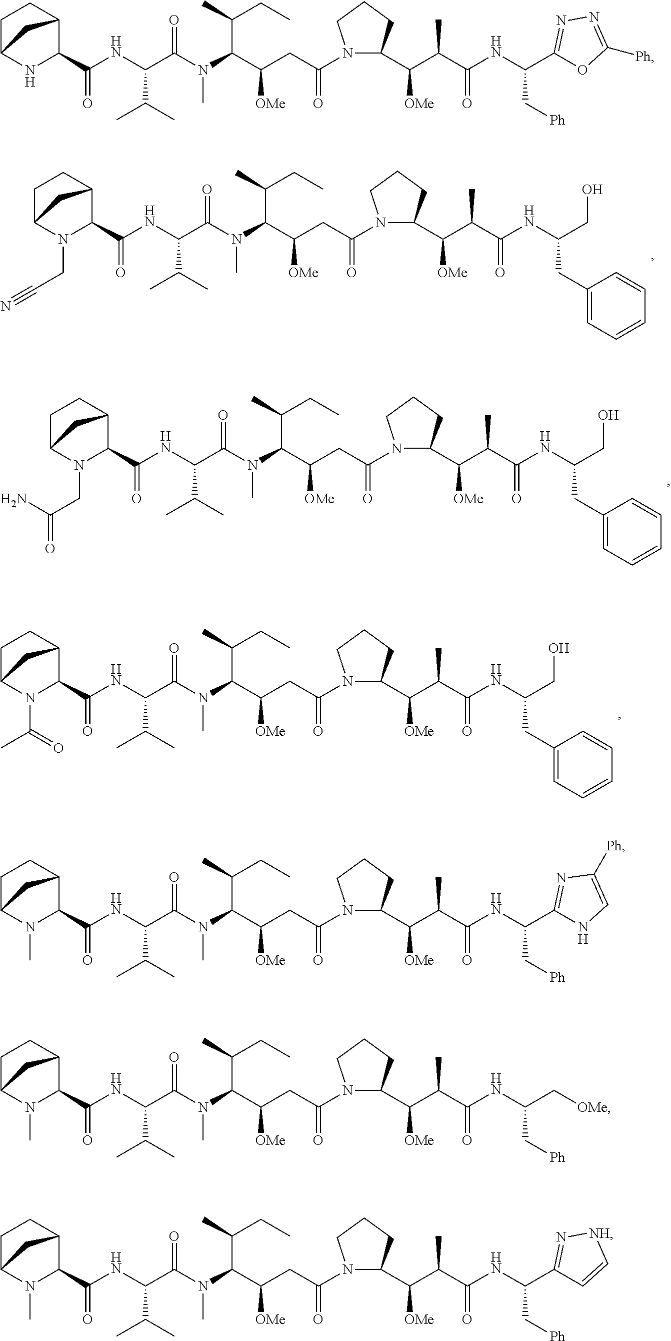

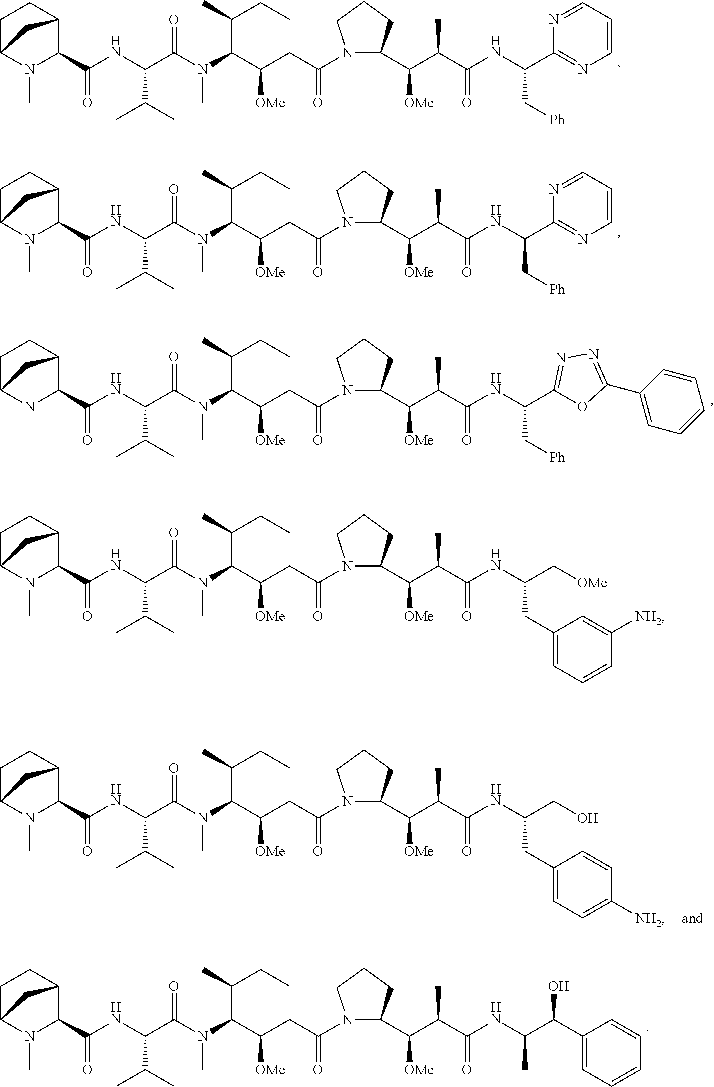

In some embodiments of this application, the antibody drug conjugate comprises a structure selected from:

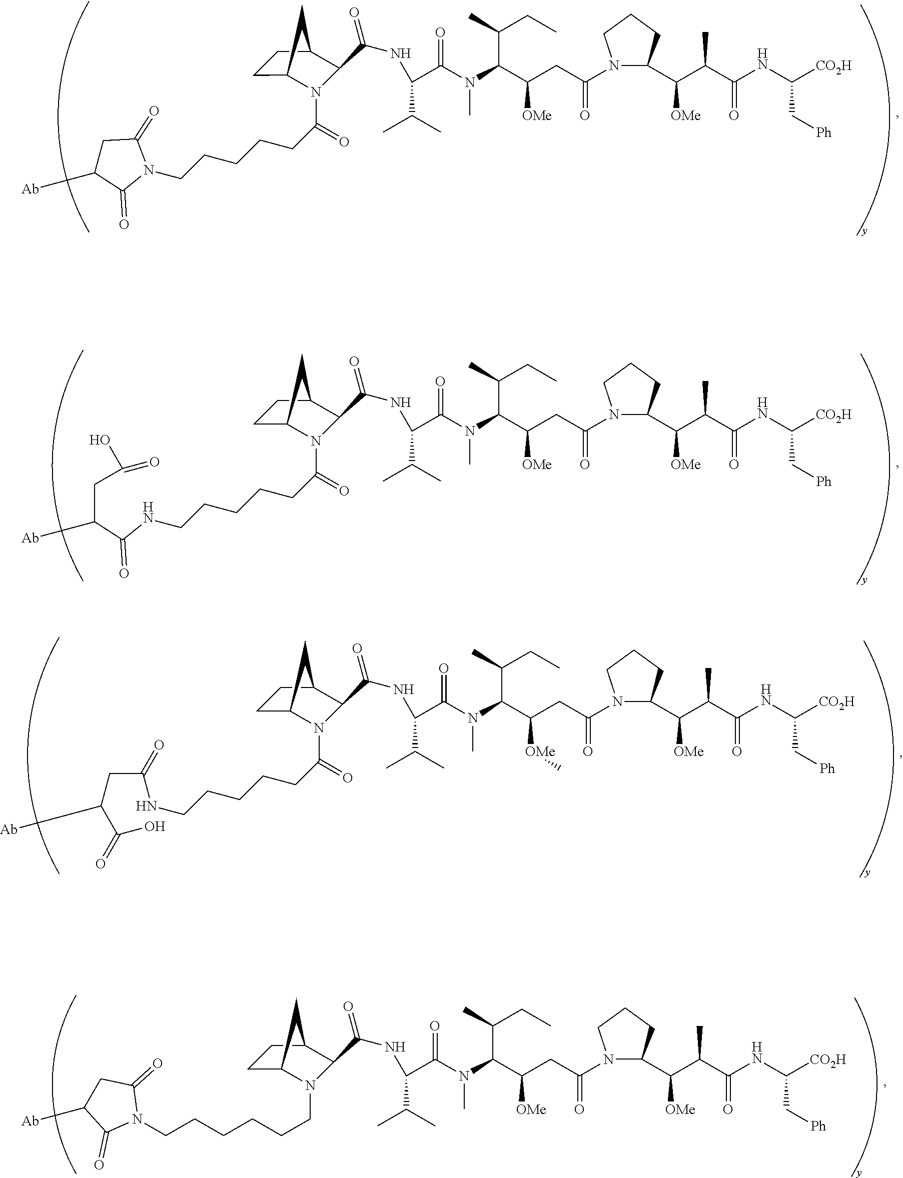

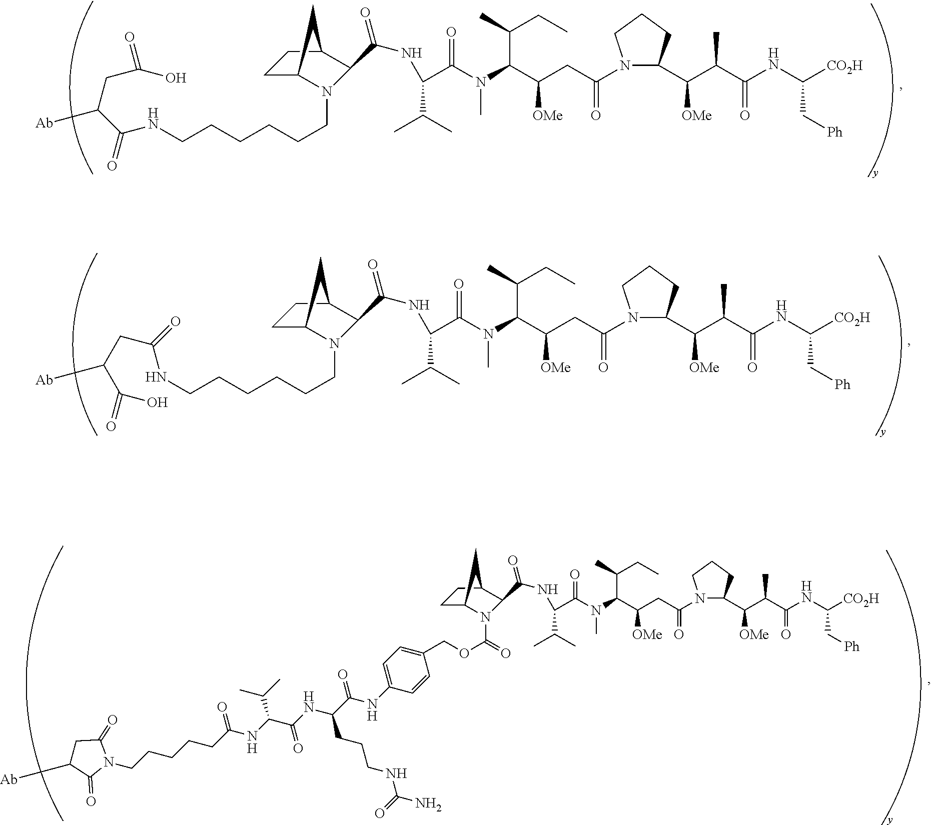

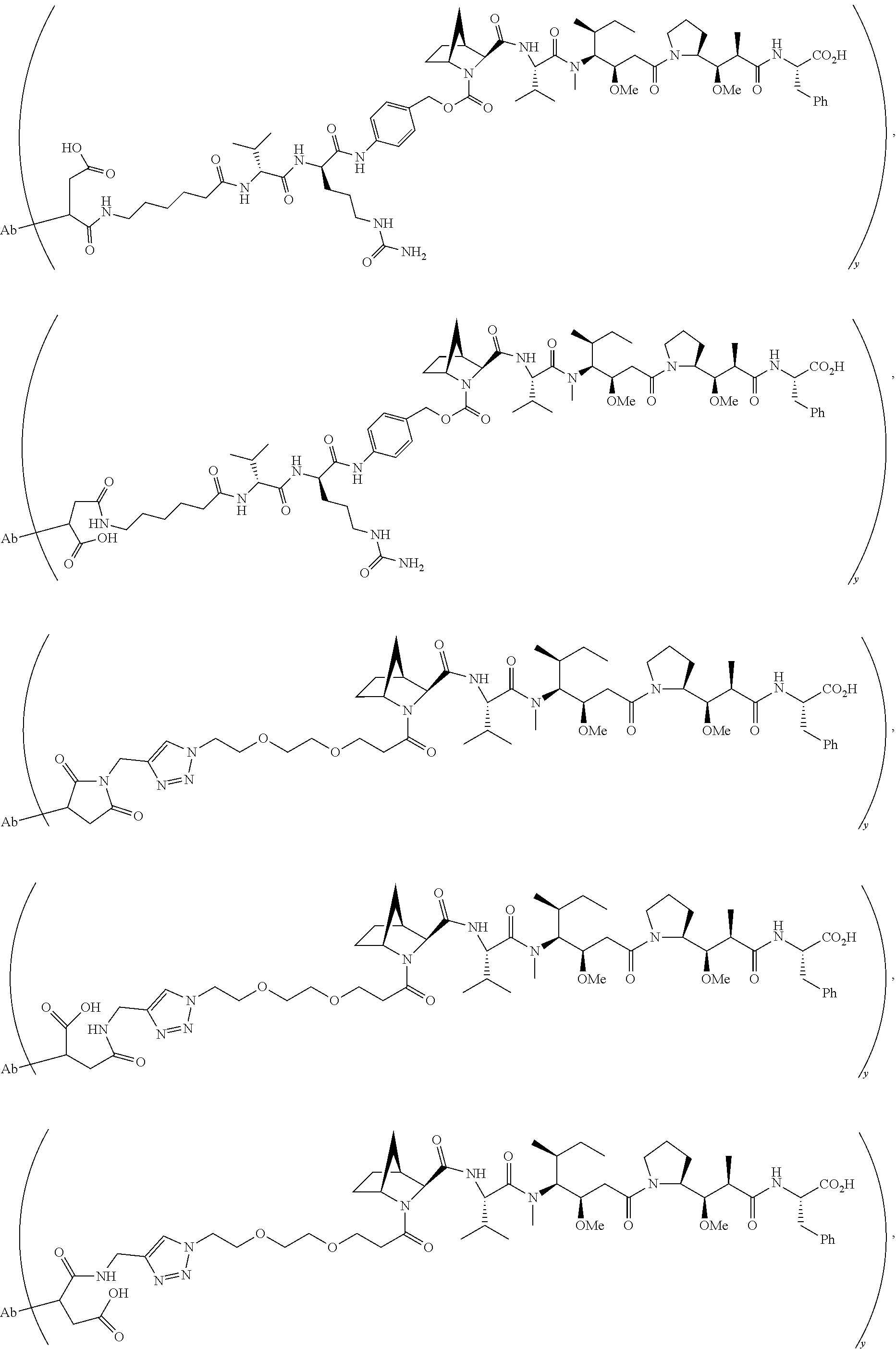

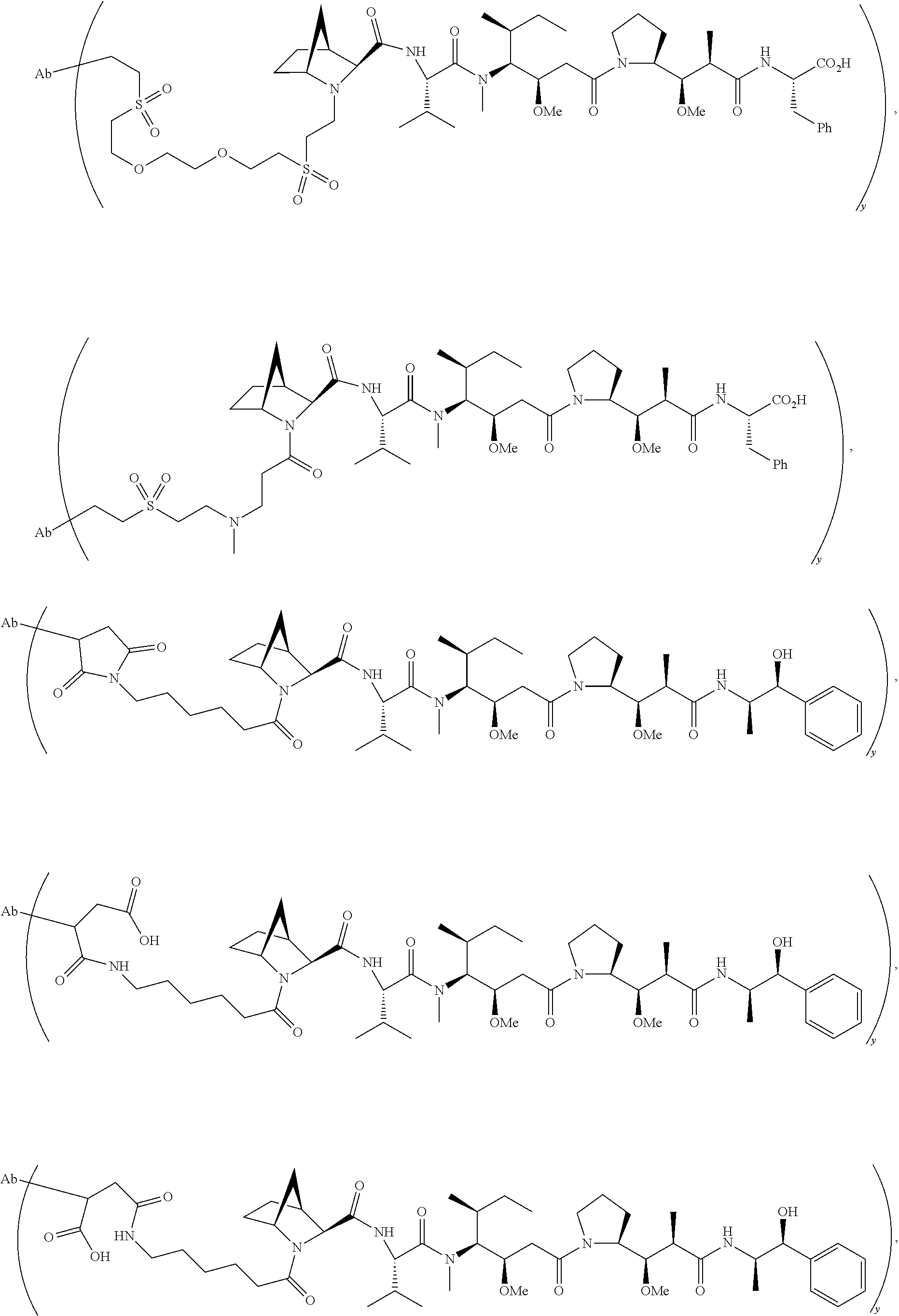

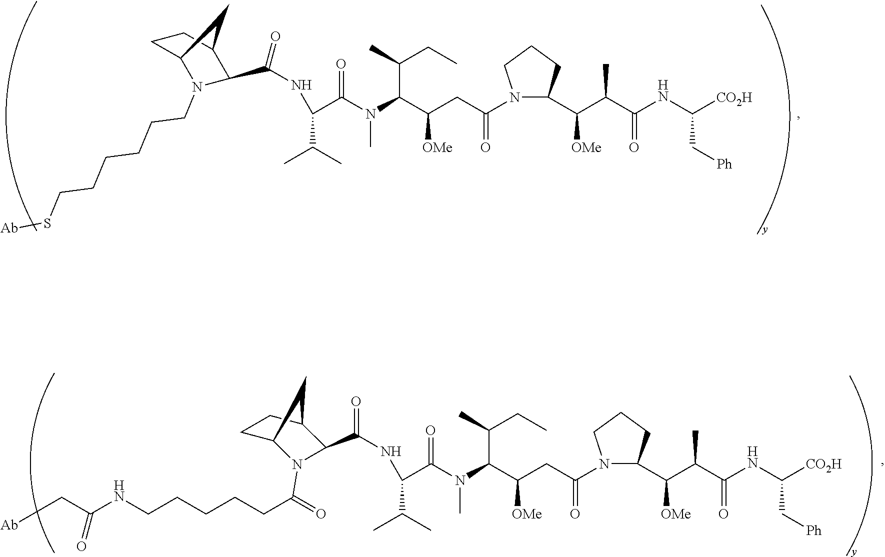

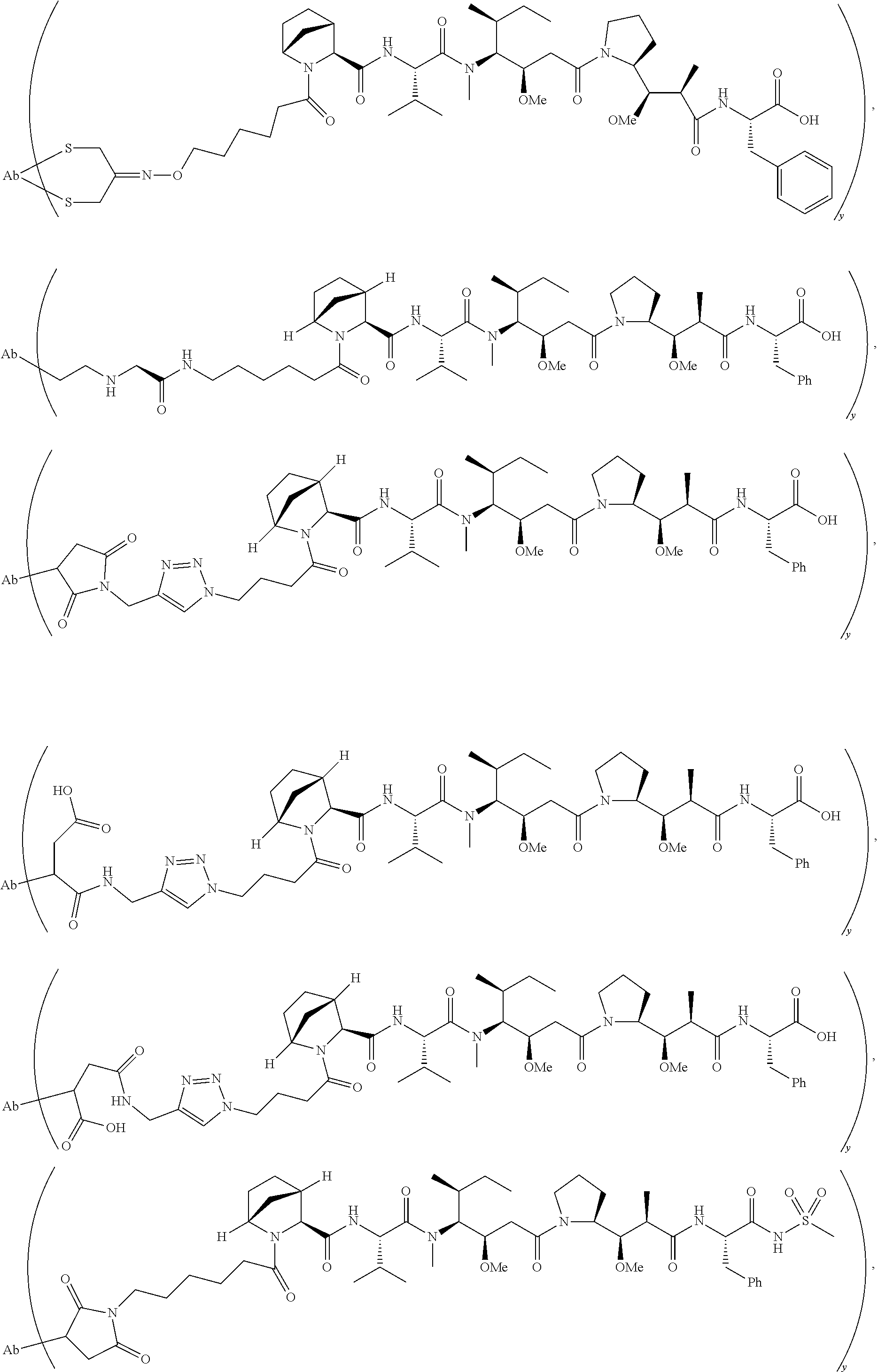

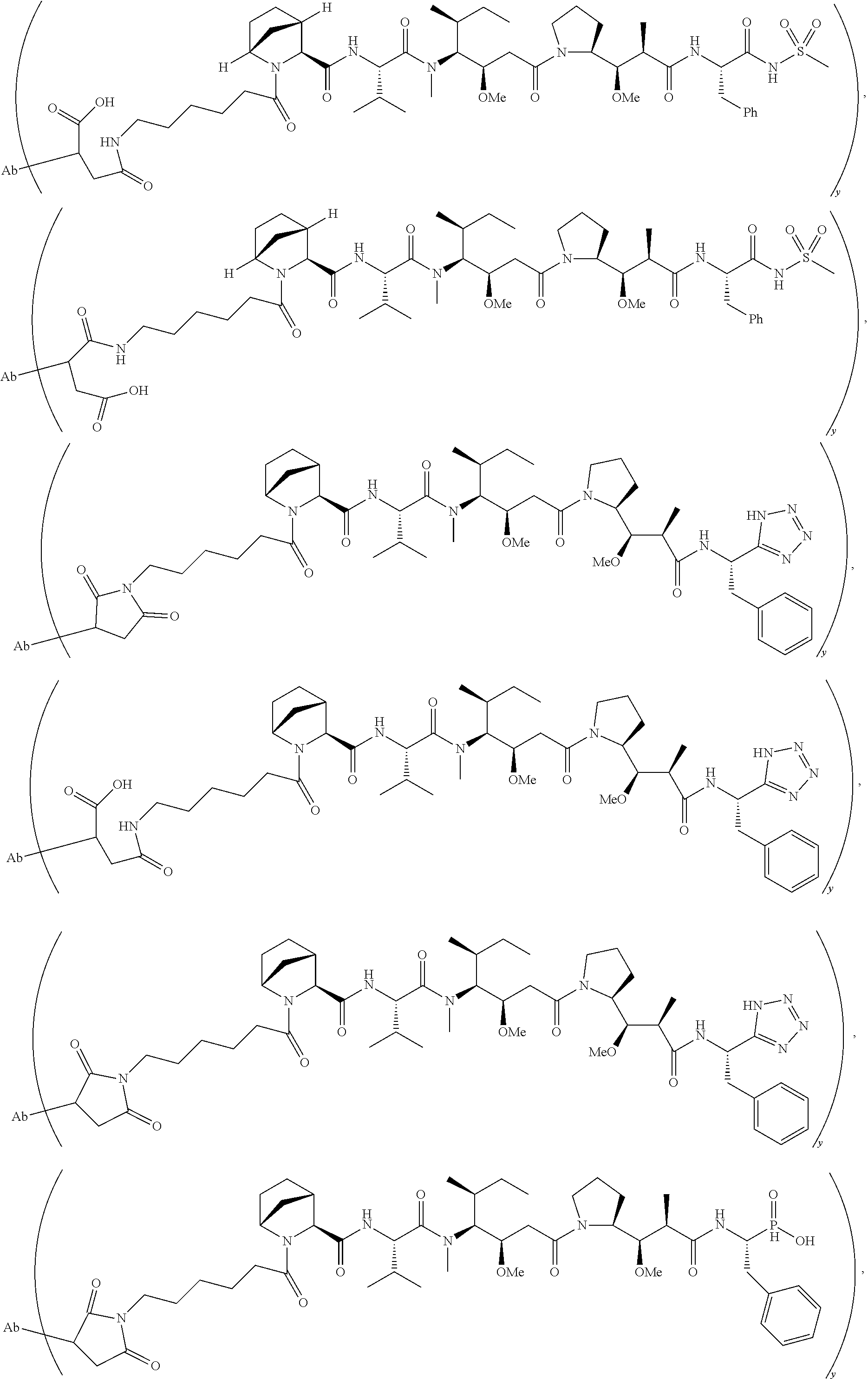

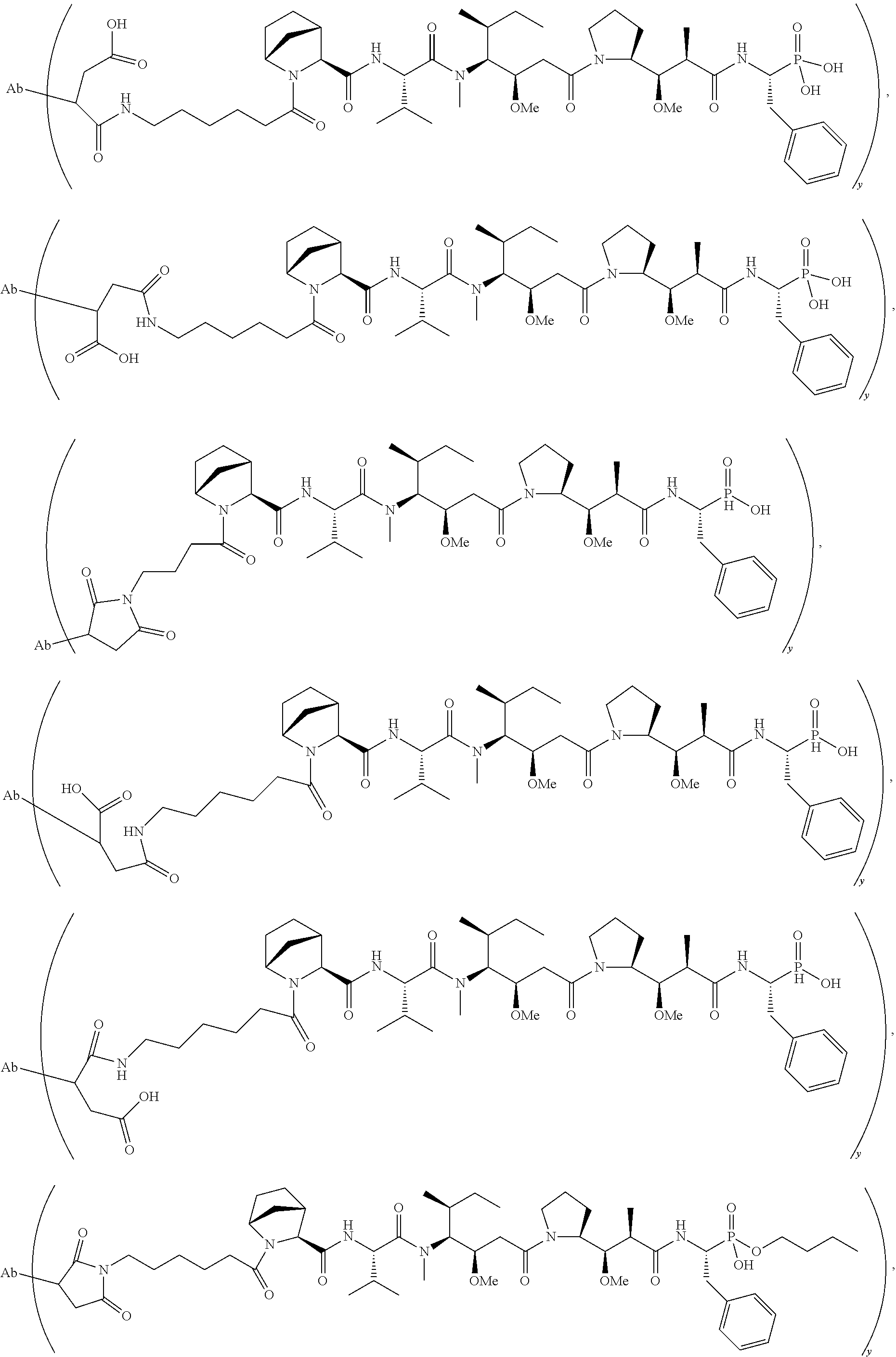

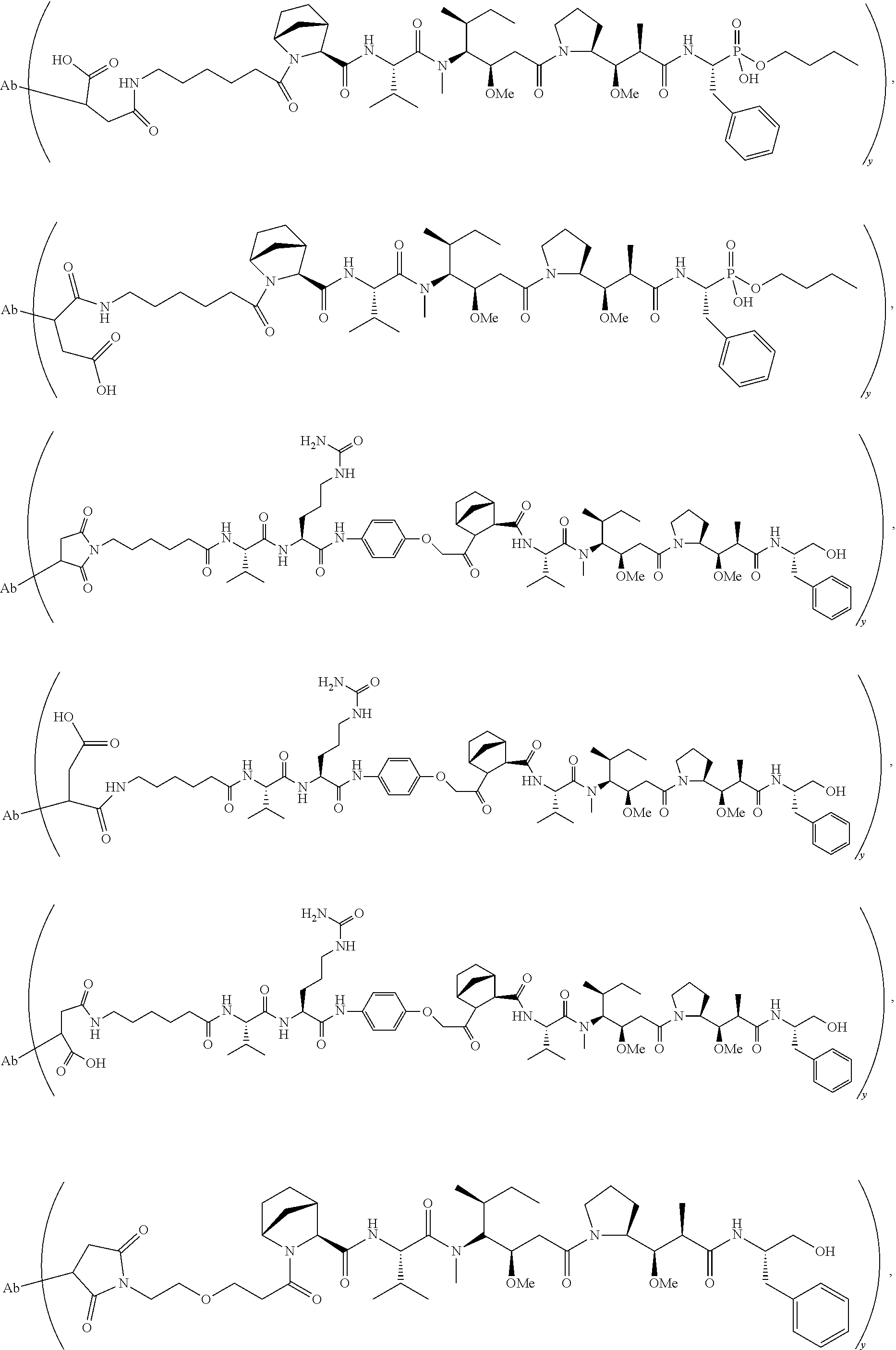

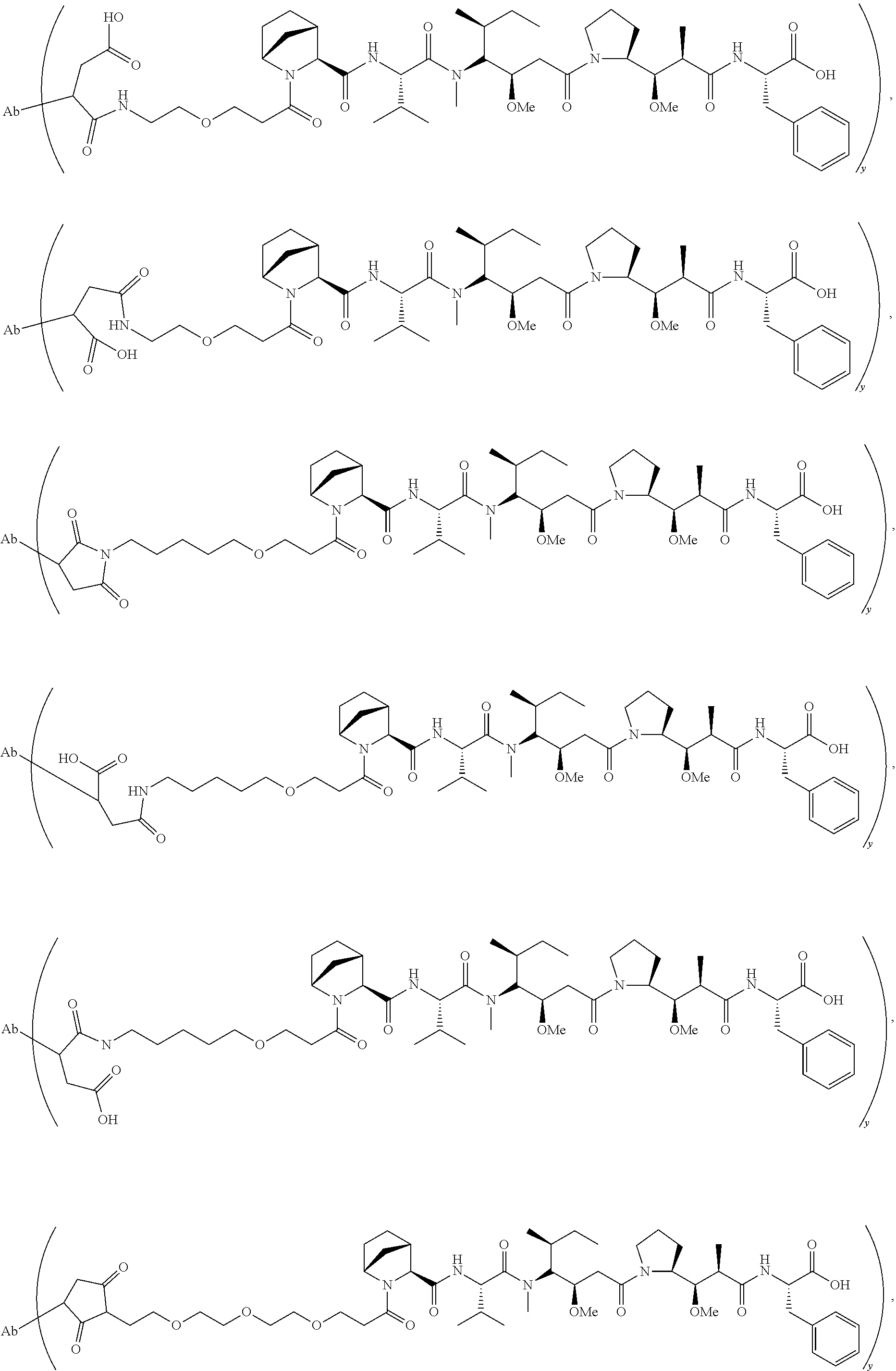

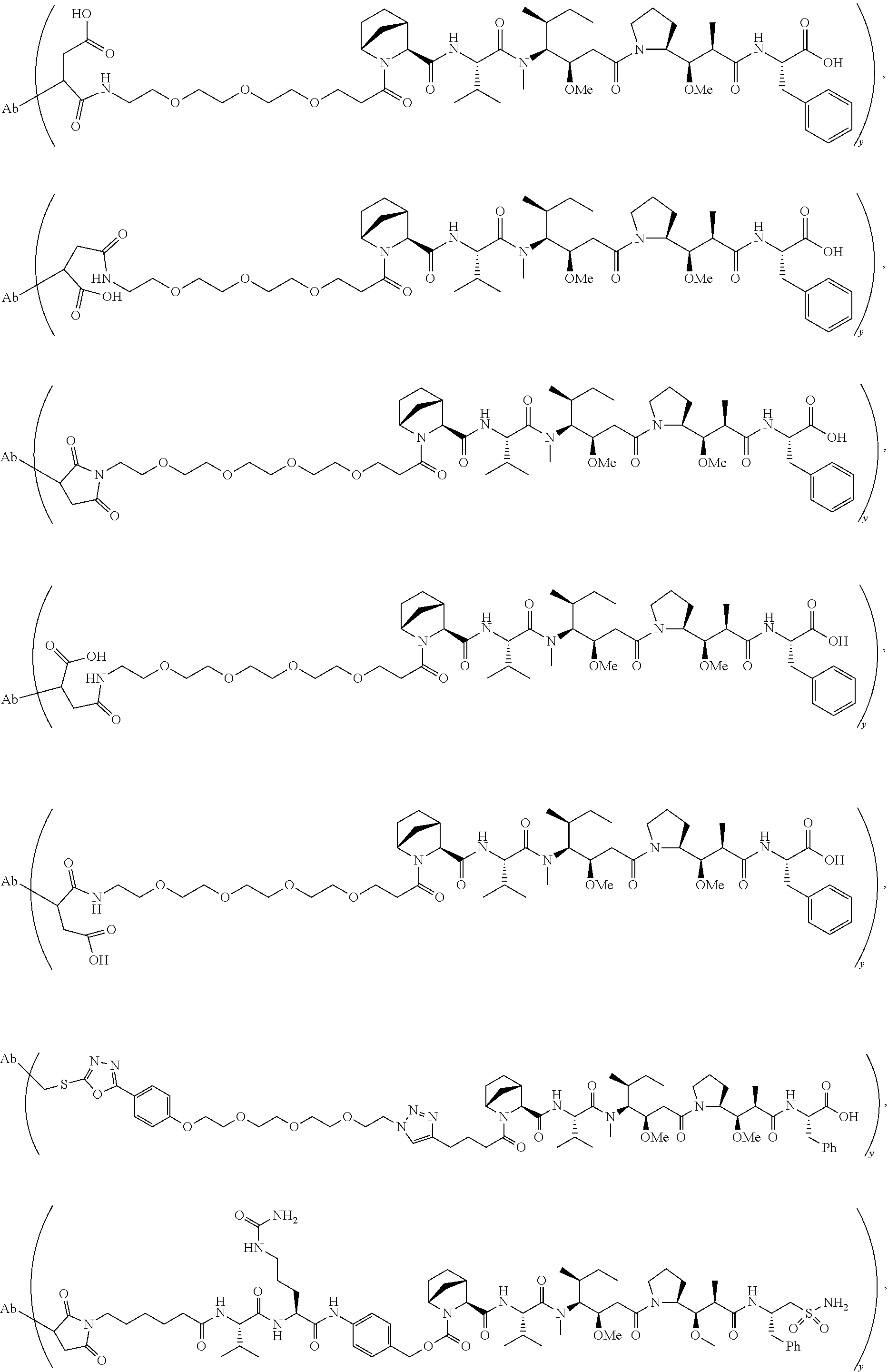

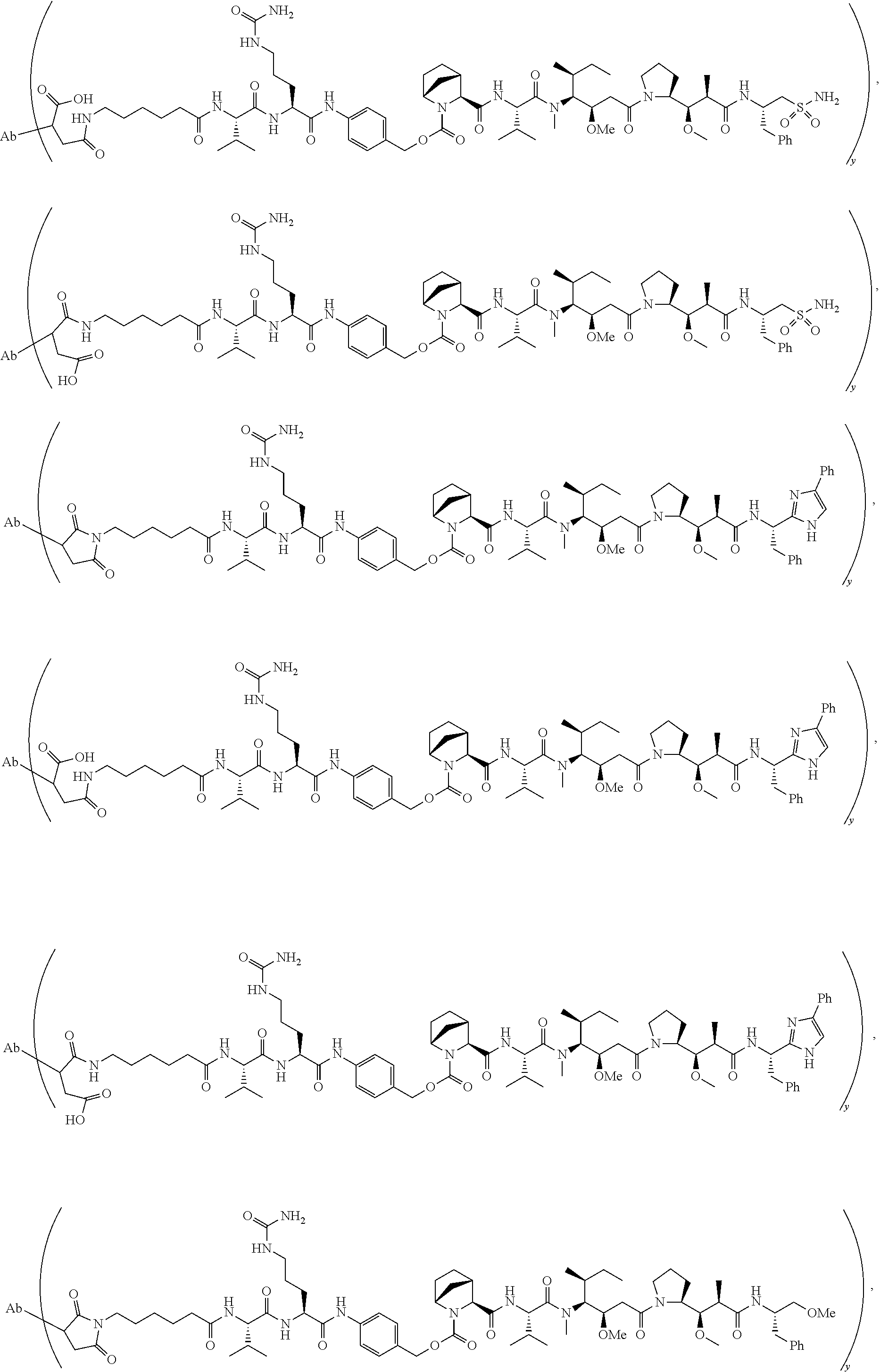

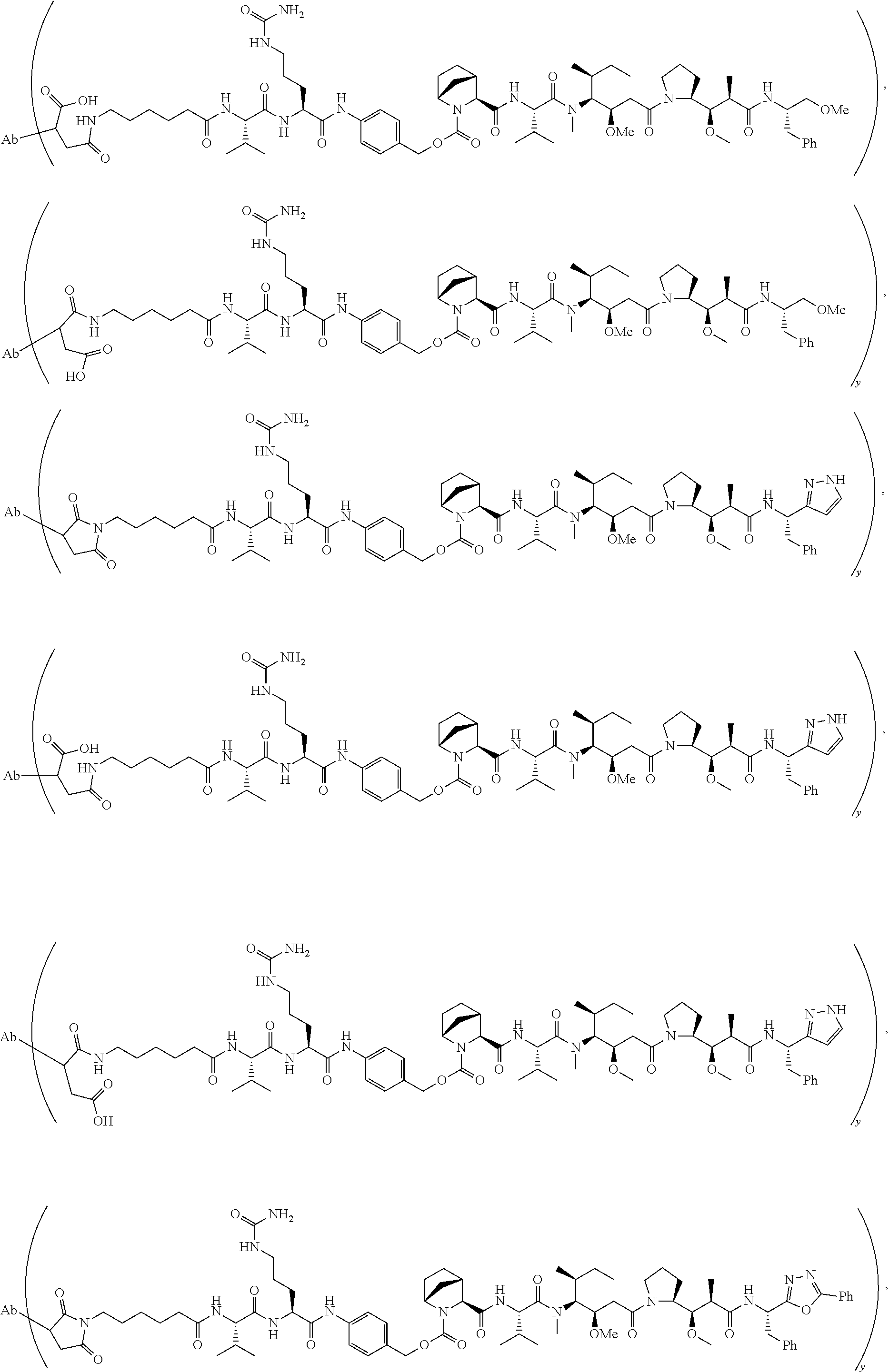

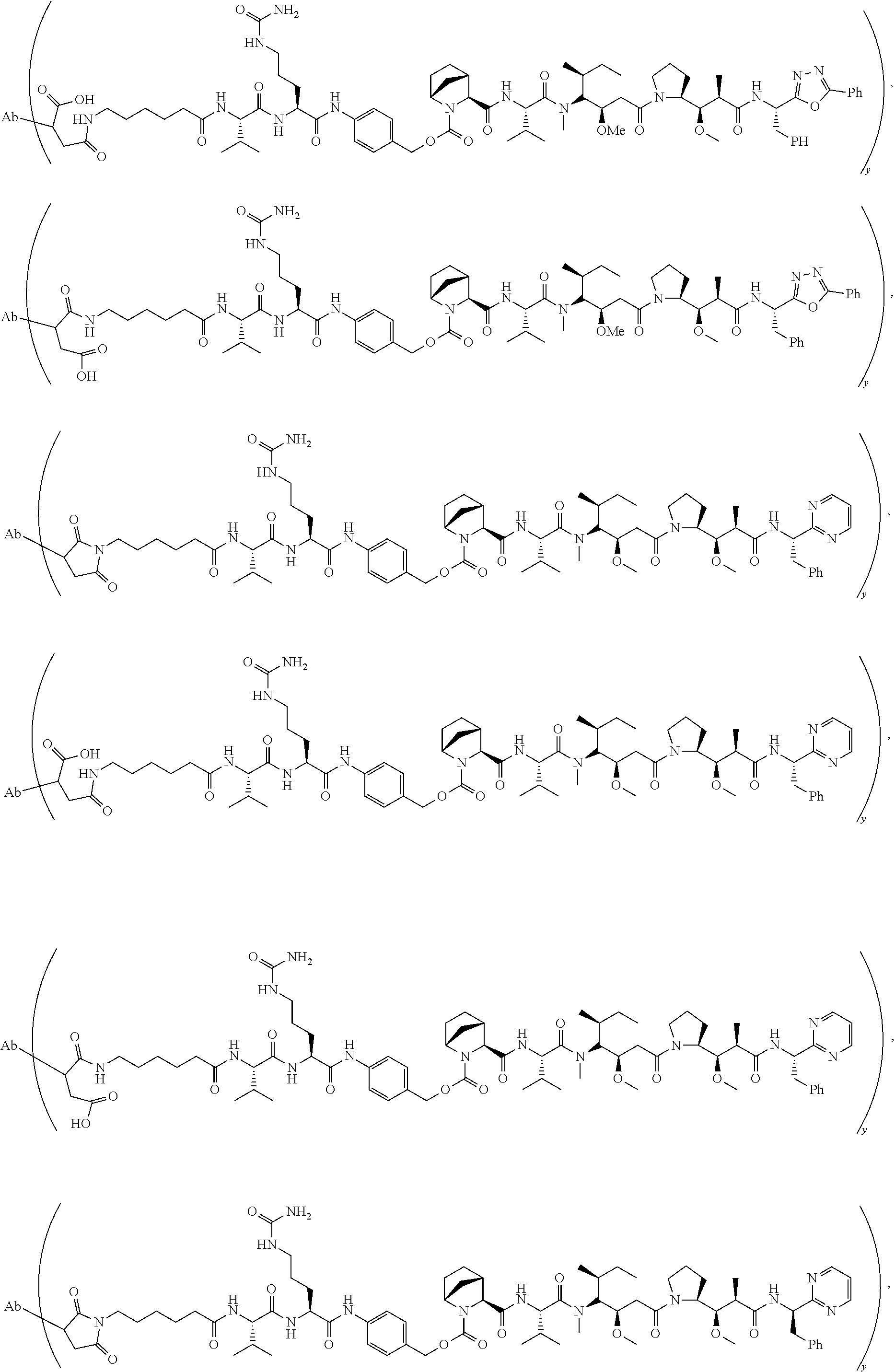

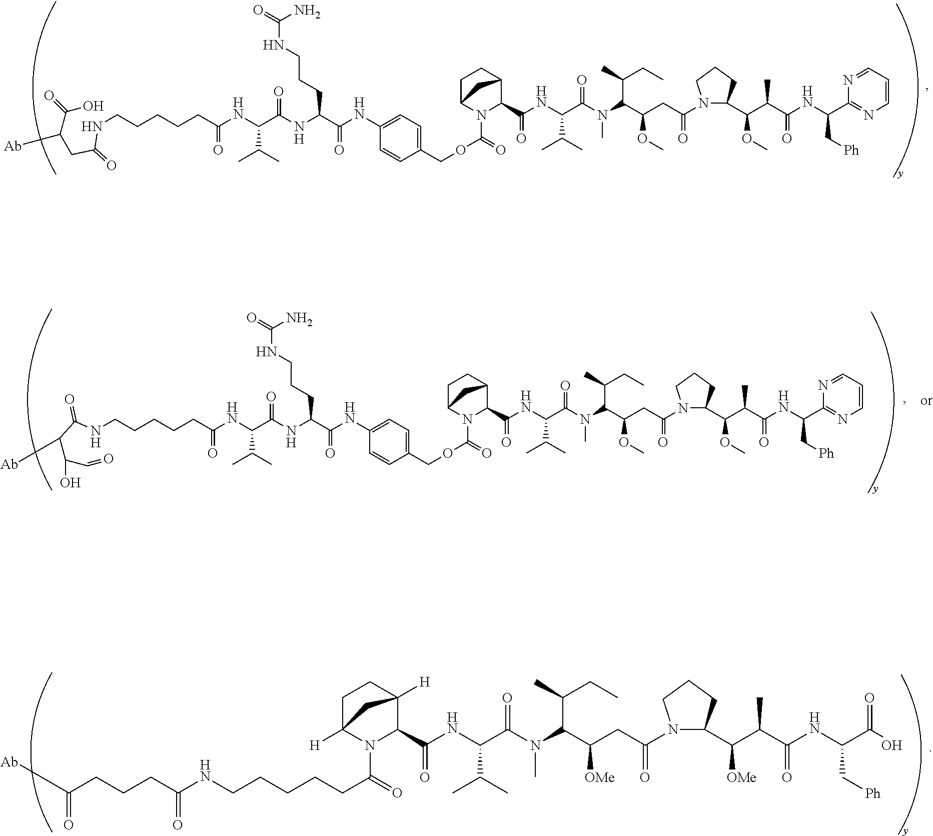

In yet other embodiments of this application, the antibody drug conjugate comprises a structure selected from:

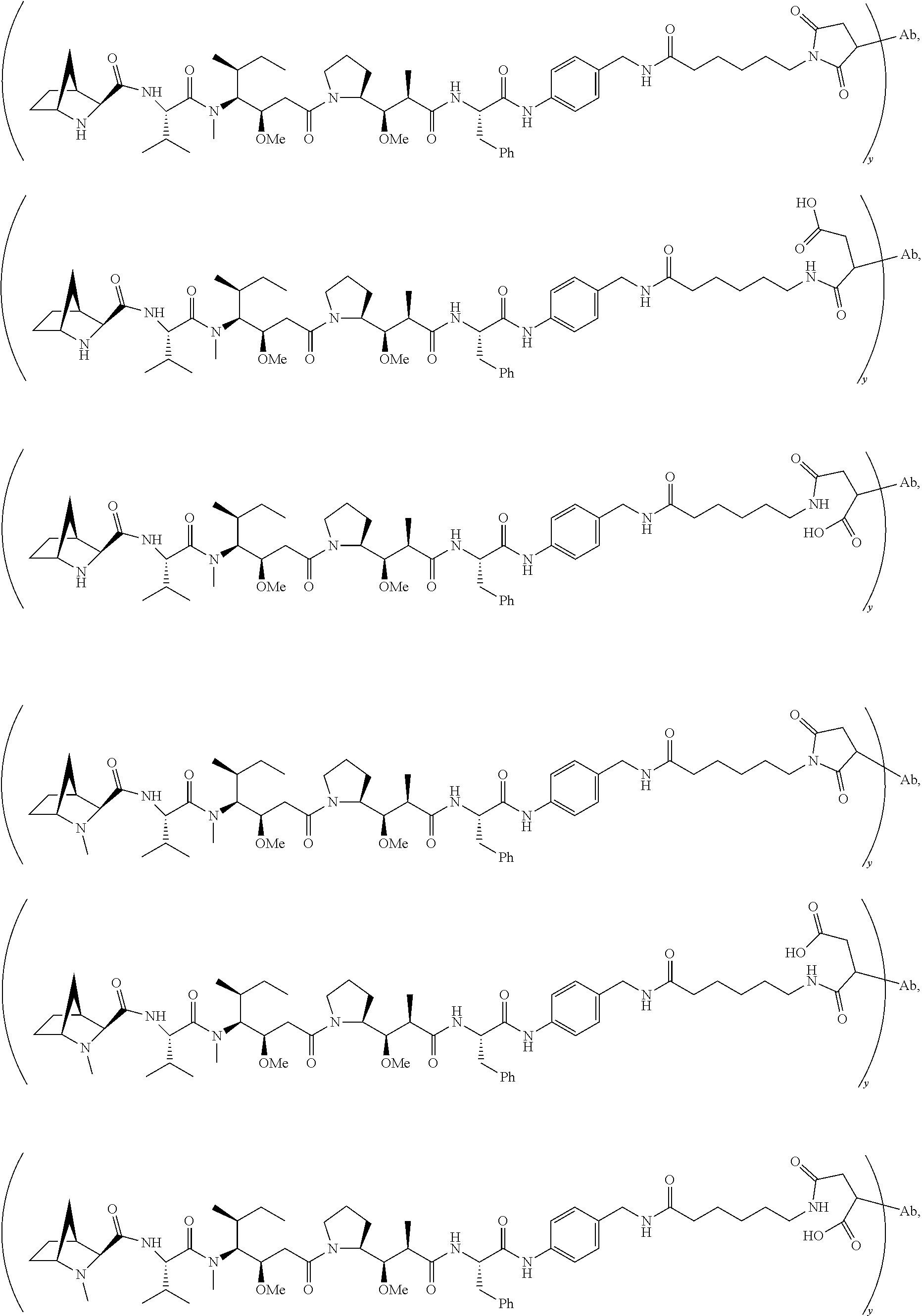

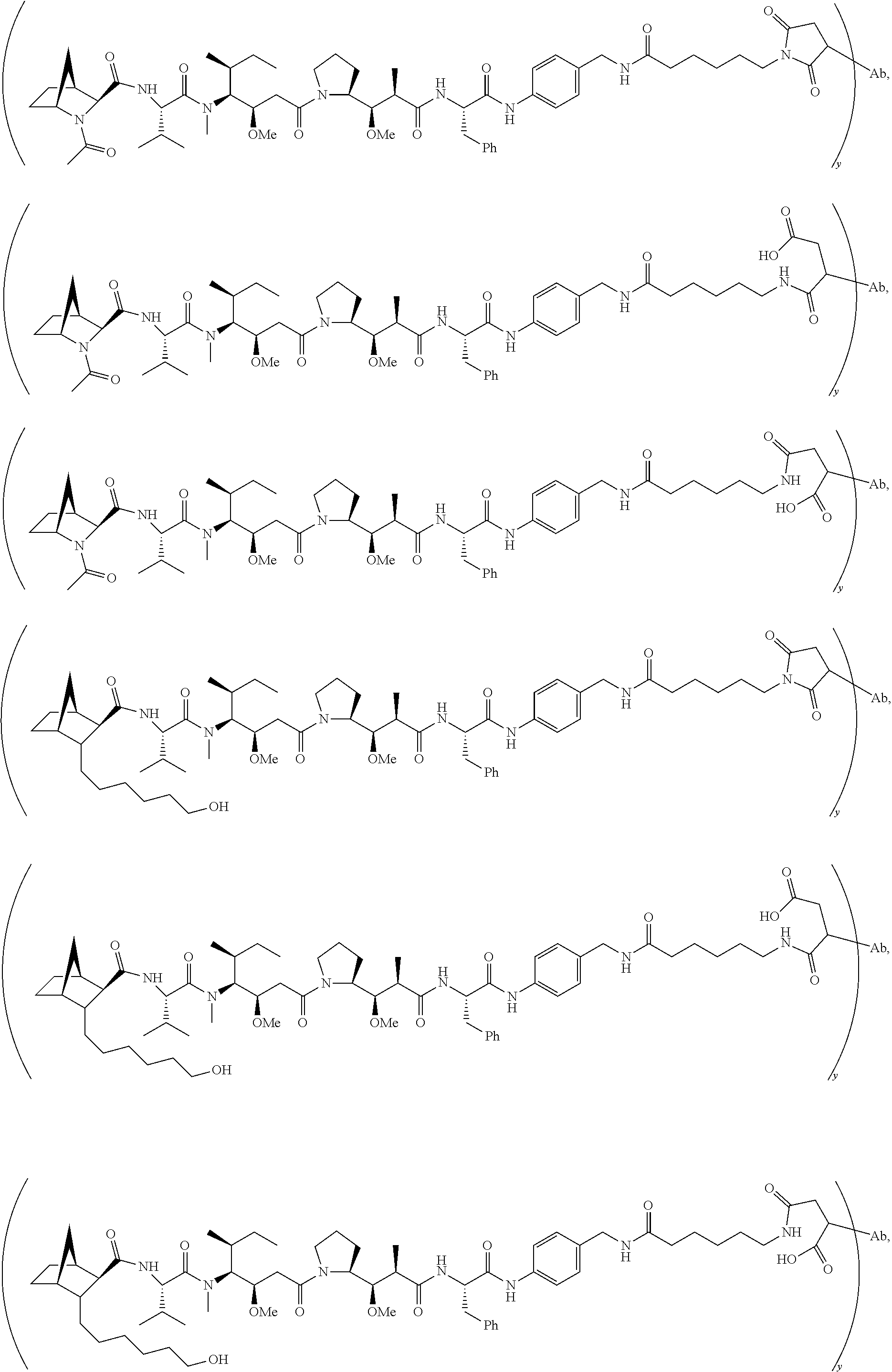

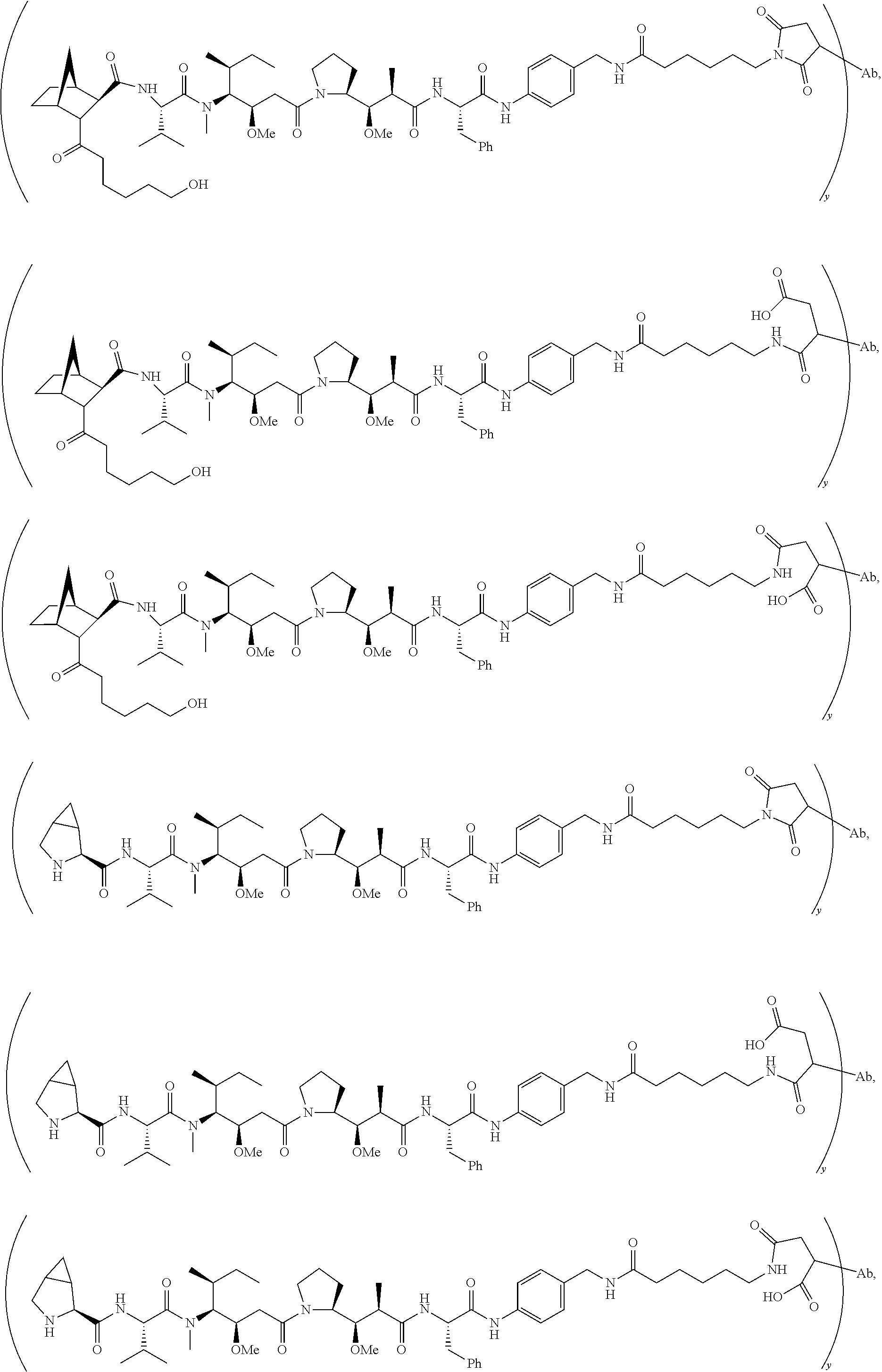

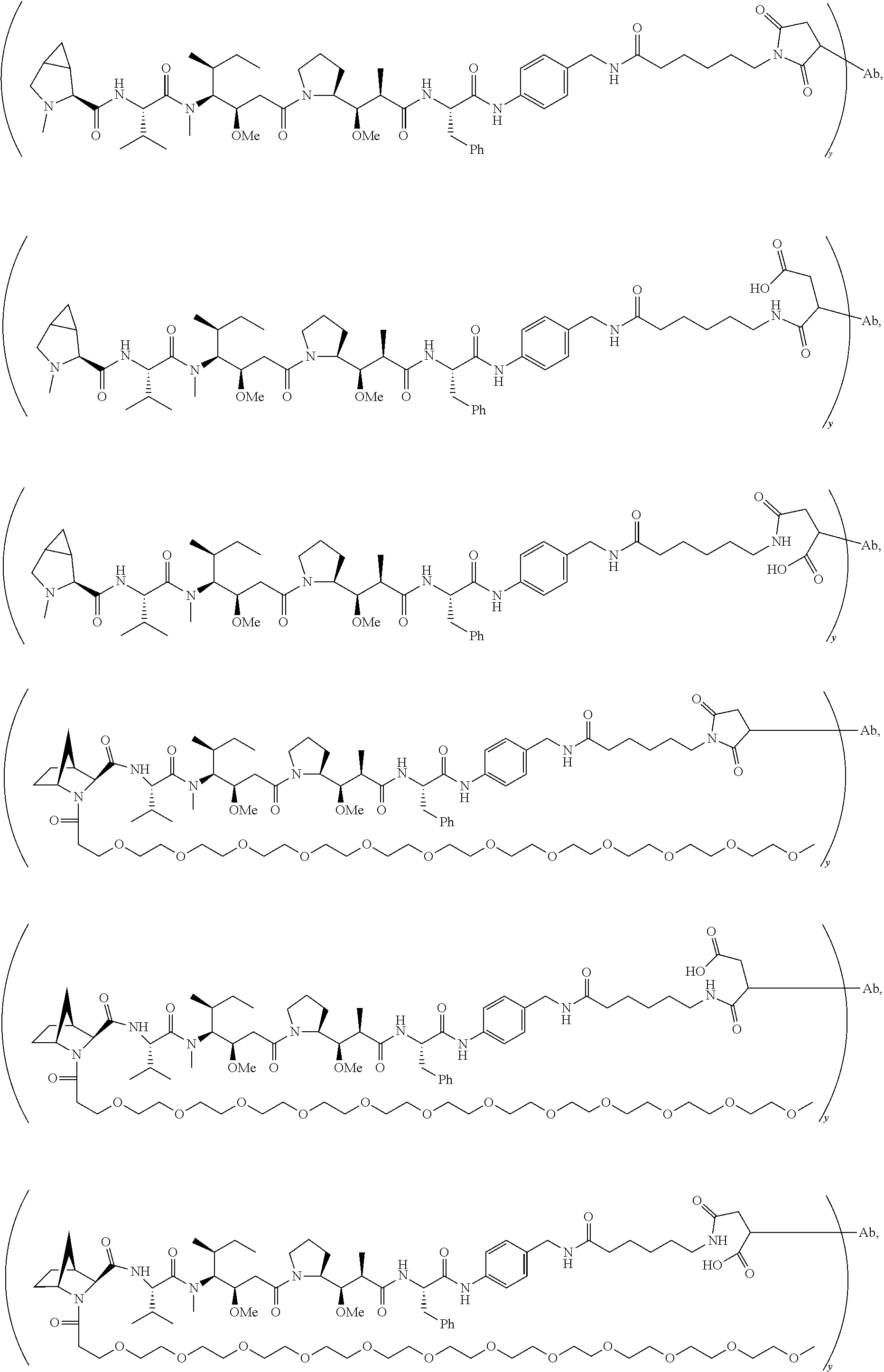

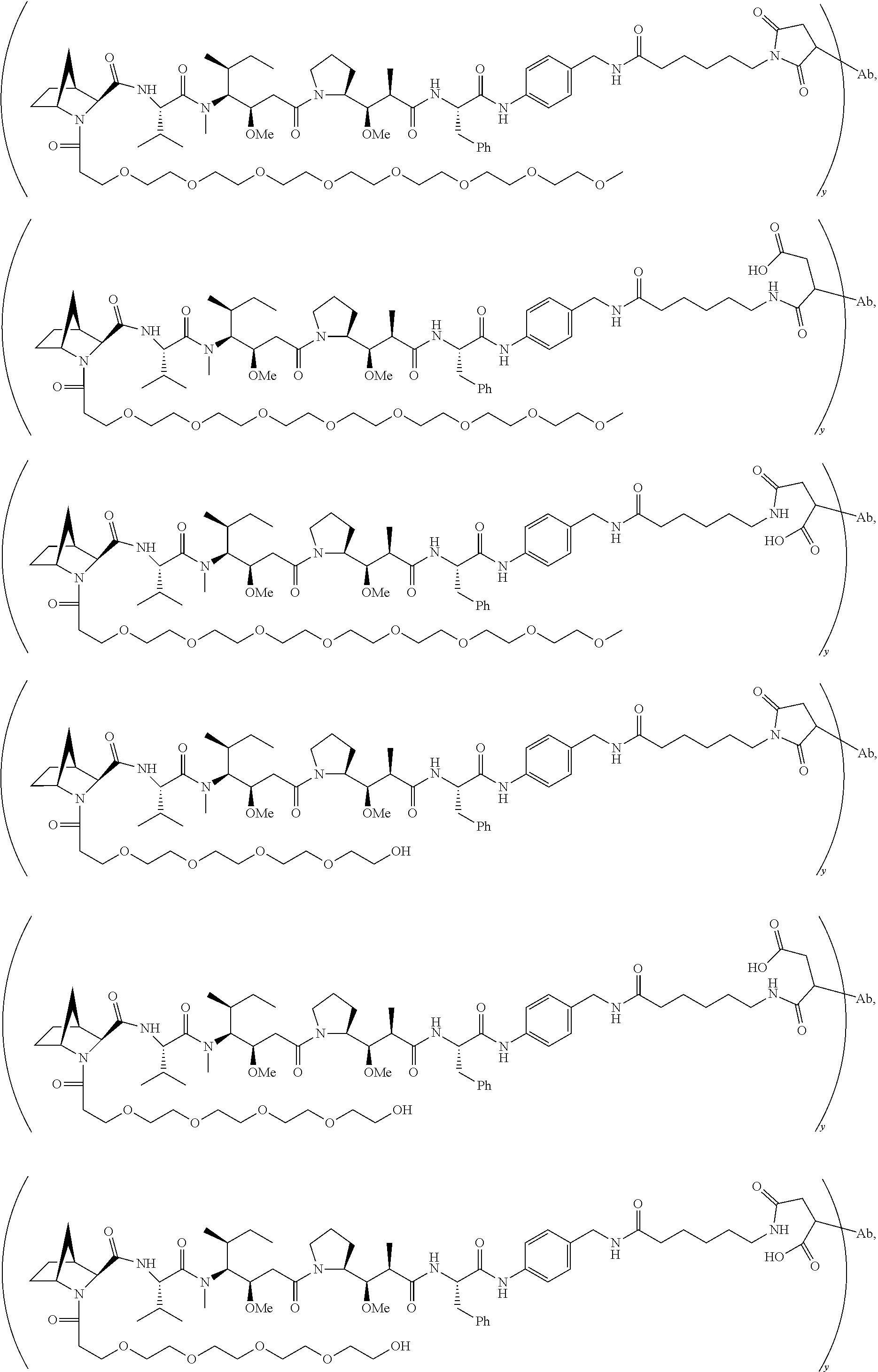

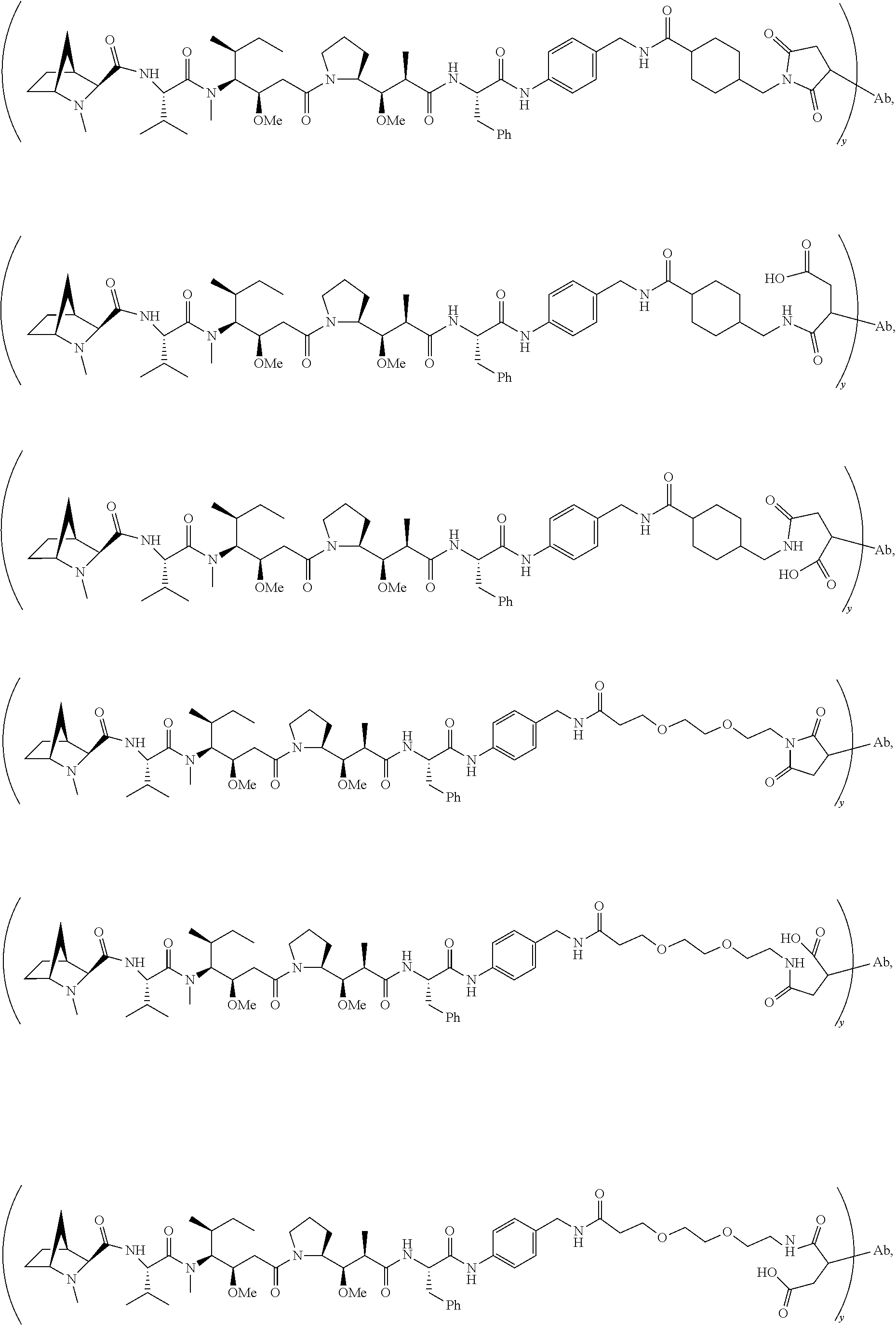

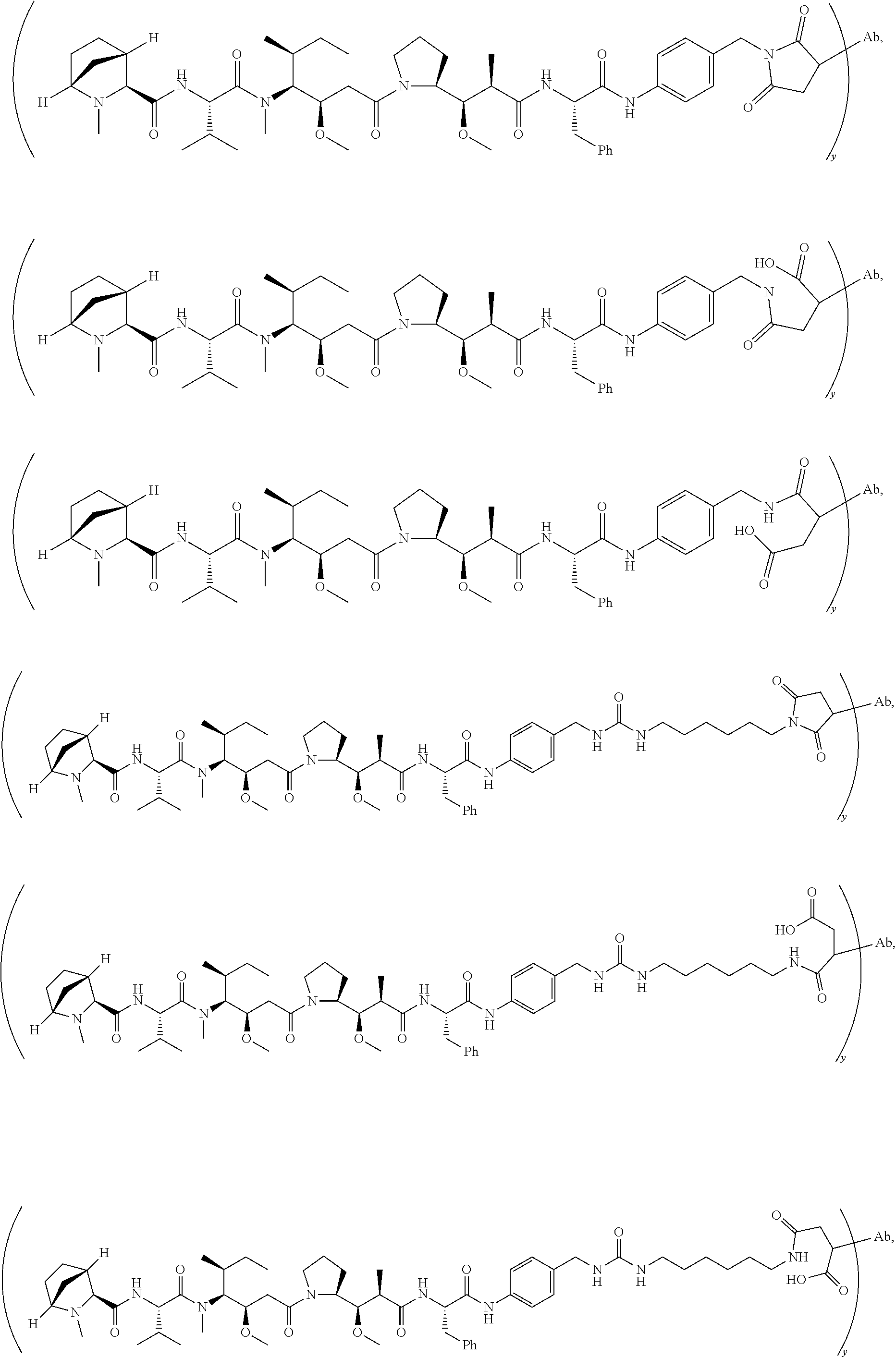

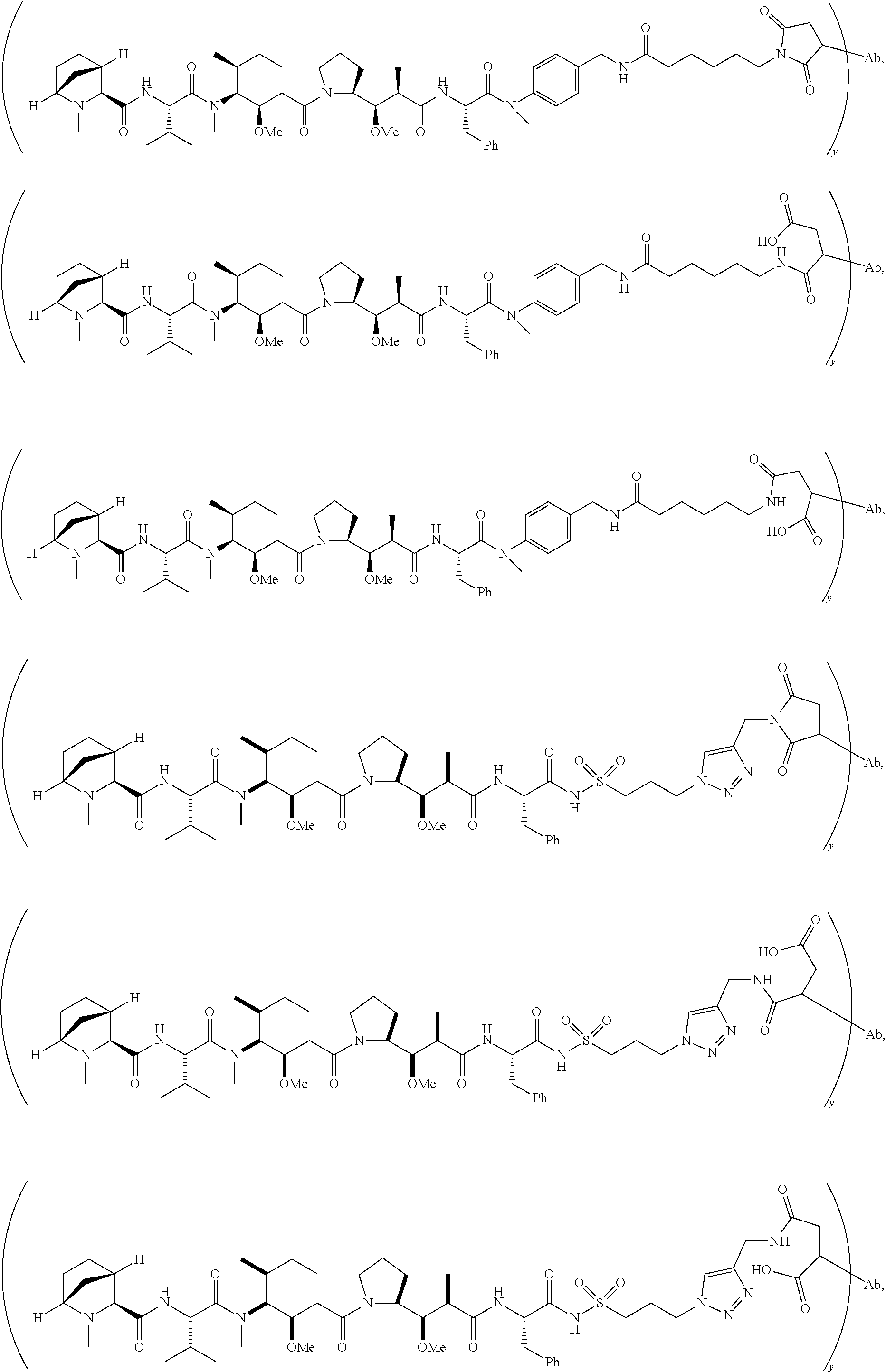

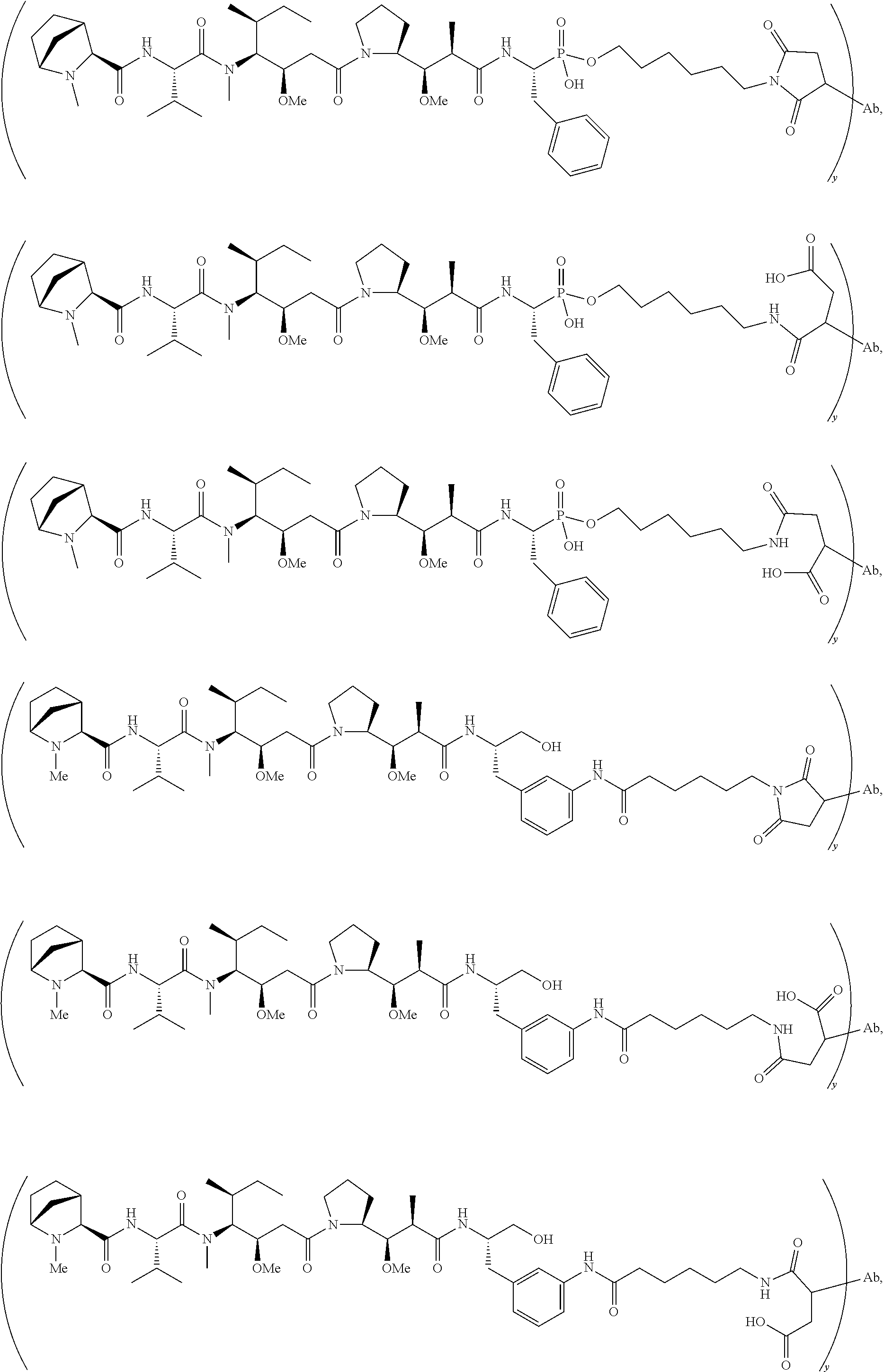

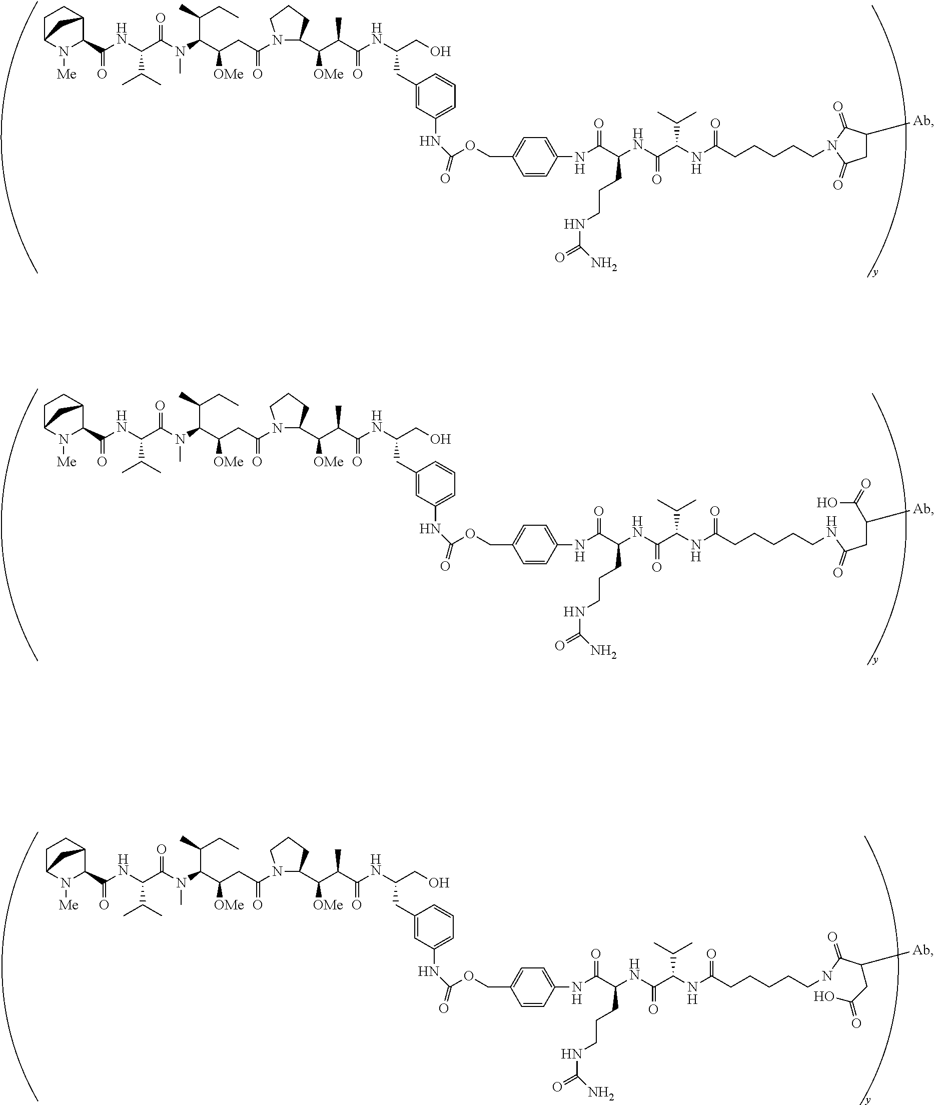

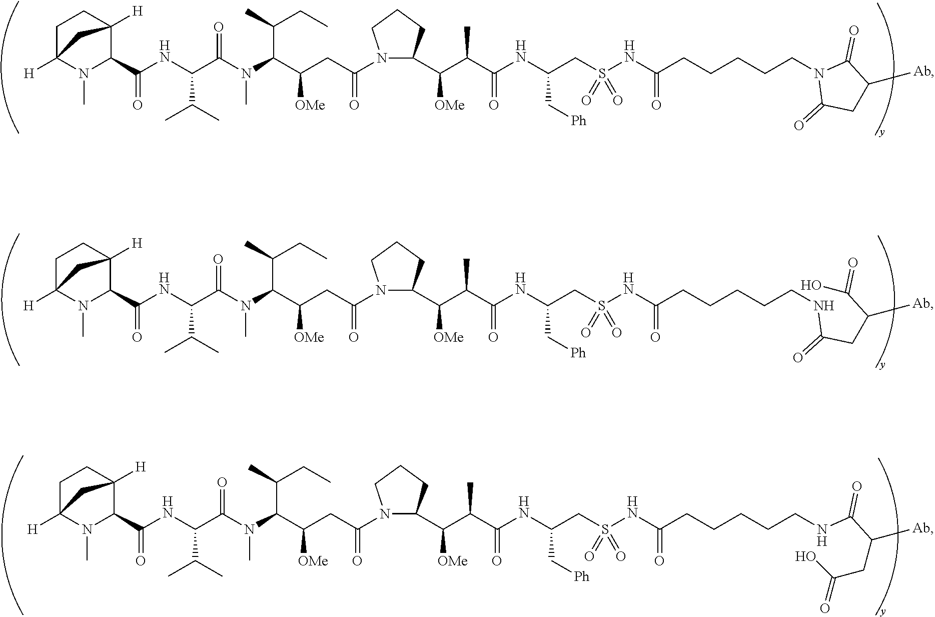

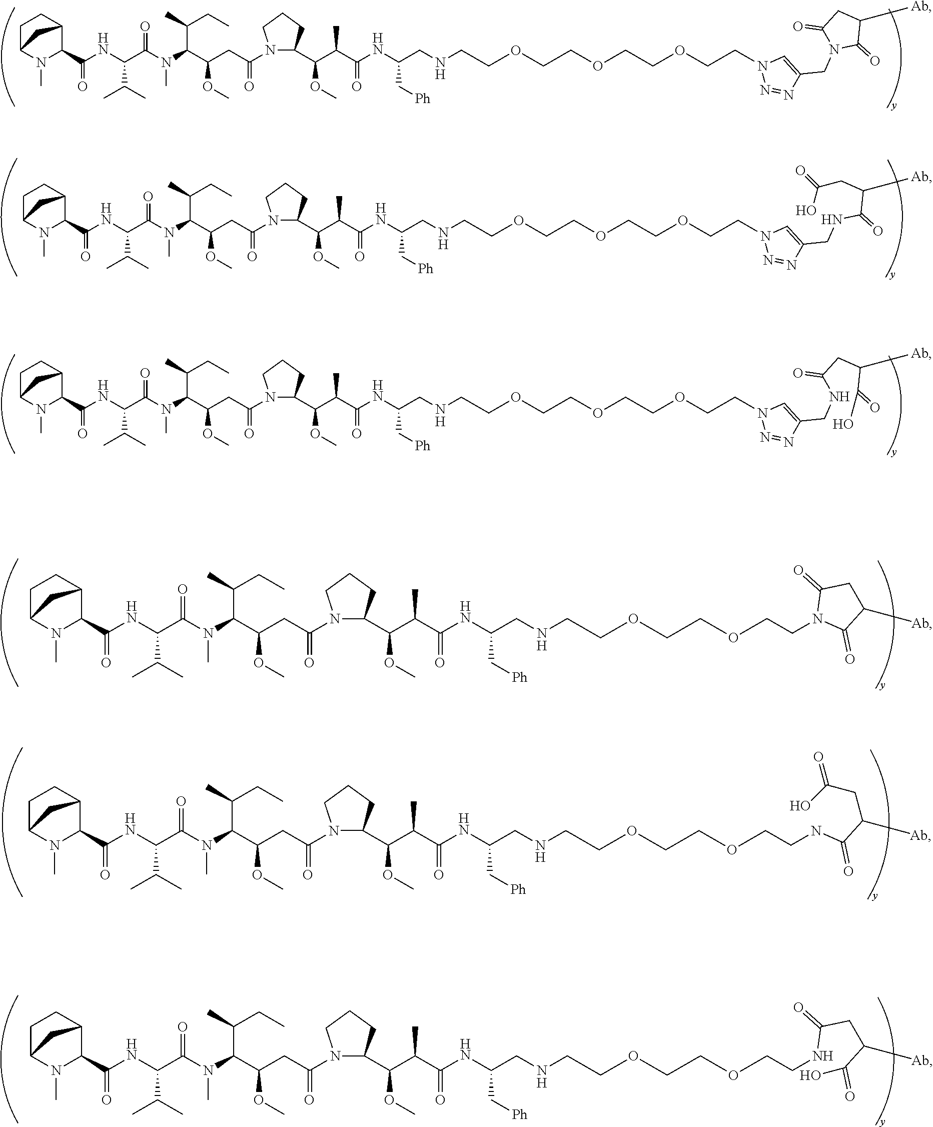

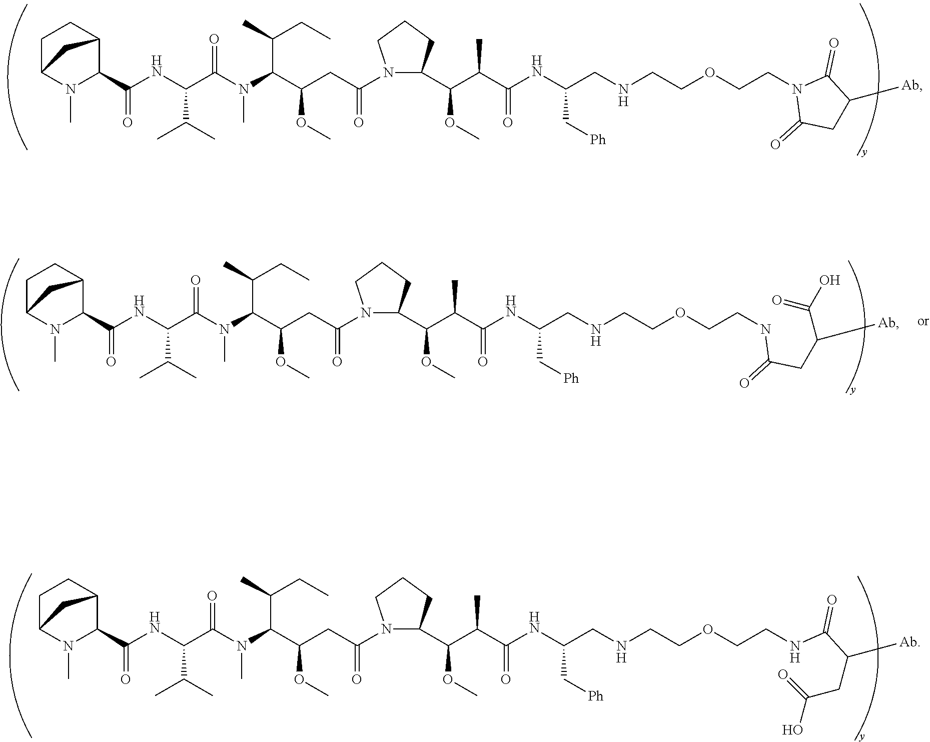

In a specific embodiment of this application, the antibody drug conjugate comprises a structure selected from:

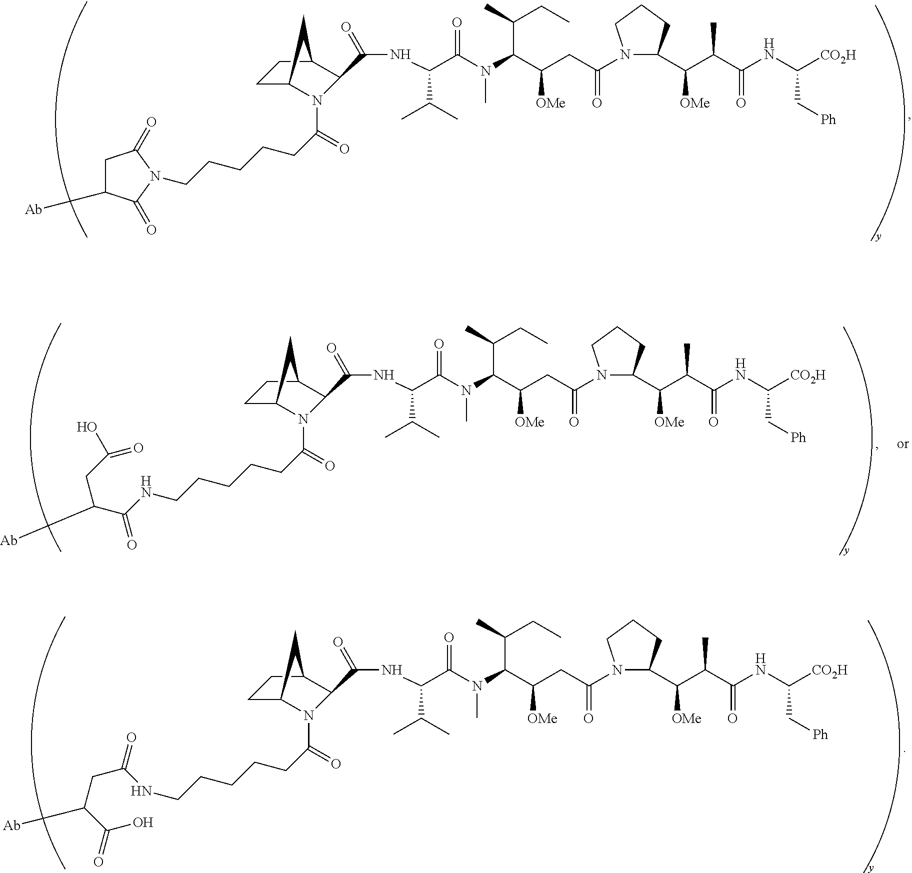

In other embodiments of this application, the antibody drug conjugate comprises a structure selected from:

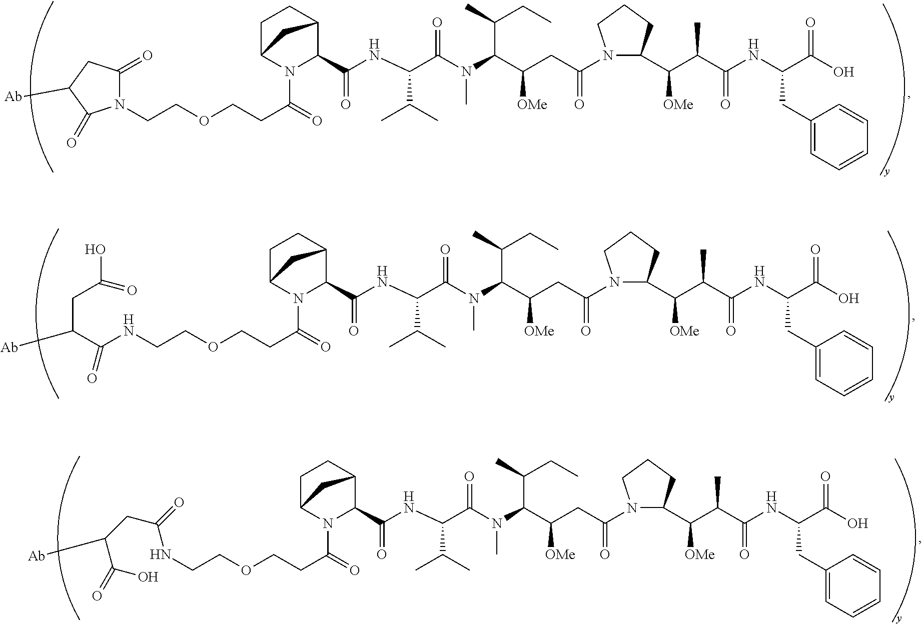

In further embodiments of this application, the antibody drug conjugate has the structure:

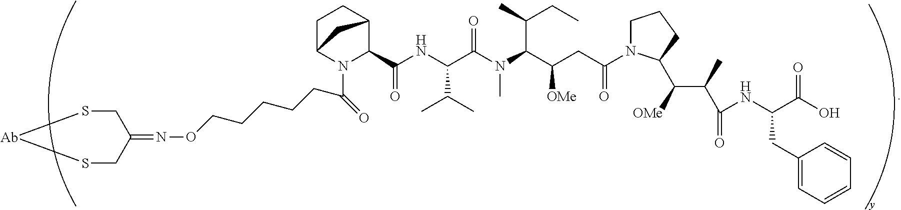

In some embodiments of this application, the antibody drug conjugate comprises an antibody or antigen binding fragment that comprises a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at positions 152 and 375, wherein said cysteine positions are numbered according to the EU system.

In other embodiments of this application, the antibody drug conjugate comprises an antibody or antigen binding fragment thereof that comprises a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at positions 152 and 375, wherein said cysteine positions are numbered according to the EU system.

In yet other embodiments of this application, the antibody drug conjugate comprises an antibody or antigen binding fragment thereof that comprises a heavy chain comprising the amino acid sequence of SEQ ID NO:130, and a light chain comprising the amino acid sequence of 19.

In some embodiments of this application, the antibody drug conjugate comprises an antibody or antigen binding fragment that comprises a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition.

In other embodiments of this application, the antibody drug conjugate comprises an antibody or antigen binding fragment thereof that comprises a heavy chain variable region (VH) comprising the amino acid sequence of SEQ ID NO:7, and a light chain variable region (VL) comprising the amino acid sequence of SEQ ID NO:17.

In yet other embodiments of this application, the antibody drug conjugate comprises an antibody or antigen binding fragment thereof that comprises a heavy chain comprising the amino acid sequence of SEQ ID NO:9, and a light chain comprising the amino acid sequence of 19.

In specific embodiments of this application, the antibody drug conjugate has a structure selected from:

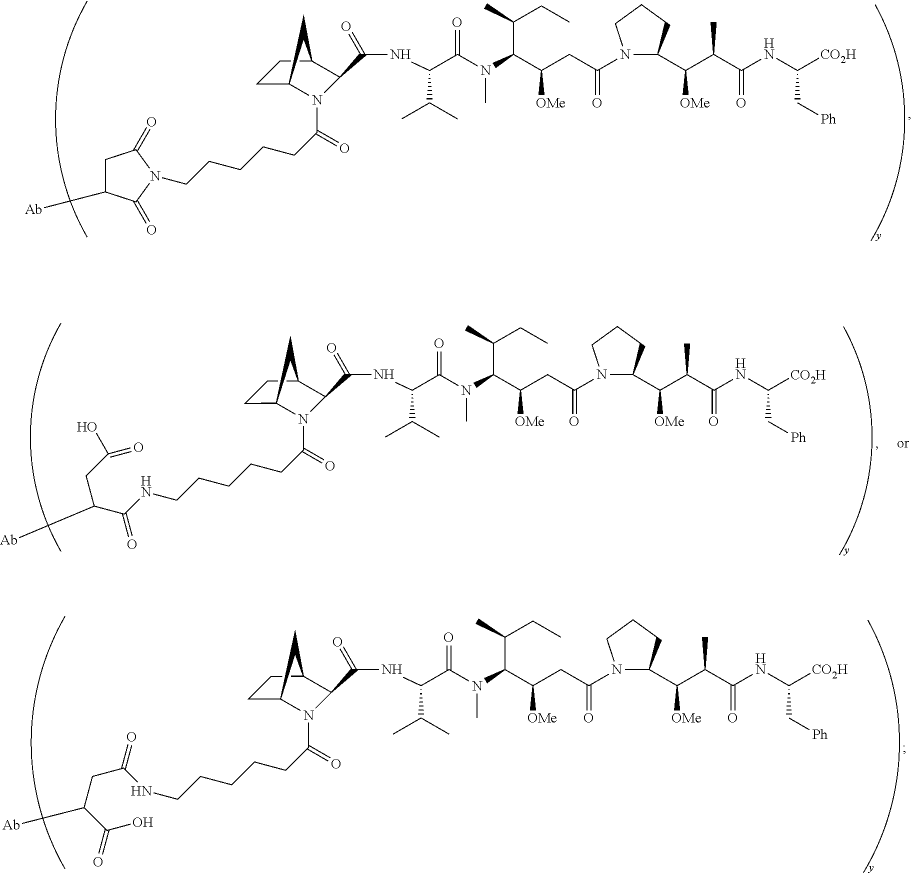

wherein Ab is an antibody comprising a heavy chain having the amino acid sequence of SEQ ID NO:130, and a light chain comprising the amino acid sequence of SEQ ID NO:19, wherein the linker-payload is conjugated to the Ab via maleimide linkage at the cysteine residues at positions 158 and 381 of SEQ ID NO 130, and wherein y is 4.

In another embodiment of this application, the antibody drug conjugate has a structure selected from:

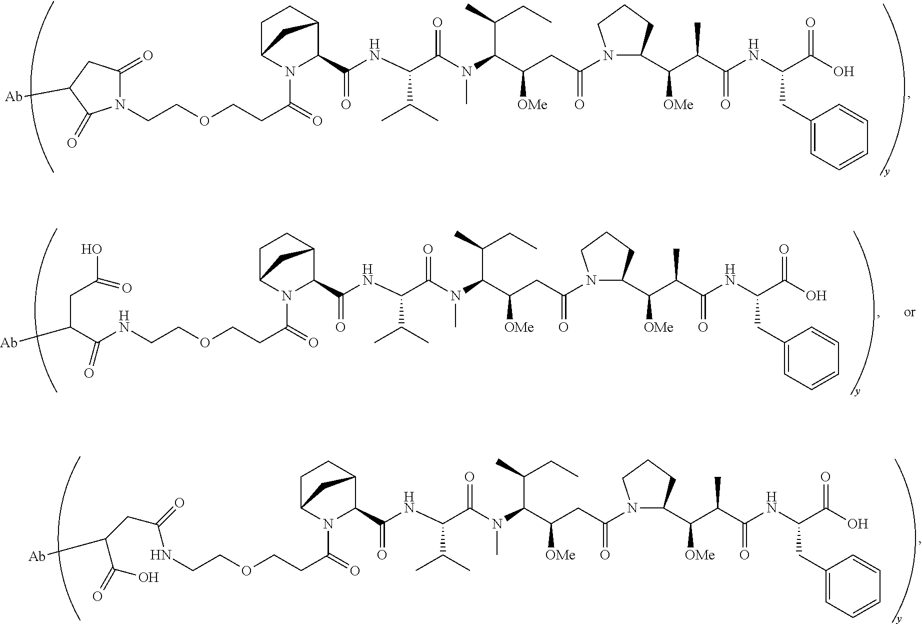

wherein Ab is an antibody comprising a heavy chain having the amino acid sequence of SEQ ID NO:130, and a light chain comprising the amino acid sequence of SEQ ID NO:19, wherein the linker-payload is conjugated to the Ab via maleimide linkage at the cysteine residues at positions 158 and 381 of SEQ ID NO 130, and wherein y is 4.

In yet another embodiment of this application, the antibody drug conjugate has the structure:

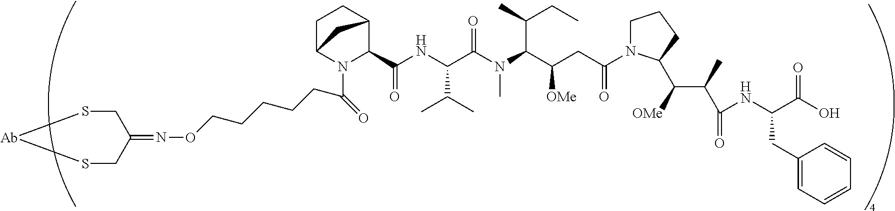

wherein Ab is an antibody comprising a heavy chain having the amino acid sequence of SEQ ID NO:9, and a light chain having the amino acid sequence of SEQ ID NO:19; and wherein the linker payload is conjugated to the Ab at the interchain disulfide bonds of the Ab.

In some embodiments of this application, in the antibody drug conjugate defined by Formula A or Formula B above, z is 1. In other embodiments, y is 4.

This application also discloses pharmaceutical compositions comprising the antibody, or antigen binding fragment thereof, as disclosed herein and a pharmaceutically acceptable carrier. In some embodiments, the pharmaceutical composition comprises the antibody drug conjugate as disclosed herein and a pharmaceutically acceptable carrier. In some embodiments, the pharmaceutical composition is prepared as a lyophilisate.

This application also discloses methods of treating cancer in a patient in need thereof, comprising administering to said patient the antibody drug conjugates or pharmaceutical compositions as disclosed herein. In some embodiments, the antibody drug conjugates or pharmaceutical compositions are administered to the patient in combination with one or more additional therapeutic compounds.

This application also discloses the antibody drug conjugates or the pharmaceutical compositions as disclosed herein for use as a medicament. In some embodiments, the antibody drug conjugates or the pharmaceutical compositions as disclosed herein are in the treatment of cancer in a patient in need thereof. This application also discloses use of the antibody drug conjugate as disclosed herein in the manufacture of a medicament for the treatment of cancer. For any of the methods treatment, antibody drug conjugates, or the uses of antibody drug conjugates as disclosed herein to treat cancer, the cancer may express P-cadherin. In some embodiments, the cancer is selected from the group consisting of adrenocortical carcinoma, bladder cancer, bone cancer, breast cancer, central nervous system atypical teratoid/rhabdoid tumors, colon cancer, colorectal cancer, embryonal tumors, endometrial cancer, esophageal cancer, gastric cancer, head and neck cancer, hepatocellular cancer, Kaposi sarcoma, liver cancer, lung cancer, including small cell lung cancer and non-small cell lung cancer, ovarian cancer, rectal cancer, rhabdomyosarcomasmall intestine cancer, soft tissue sarcoma, squamous cell carcinoma, squamous neck cancer, stomach cancer, uterine cancer, vaginal cancer, and vulvar canceradrenocortical carcinoma, bladder cancer, bone cancer, breast cancer, central nervous system atypical teratoid/rhabdoid tumors, colon cancer, colorectal cancer, embryonal tumors, endometrial cancer, esophageal cancer, gastric cancer, head and neck cancer, hepatocellular cancer, Kaposi sarcoma, liver cancer, lung cancer, including small cell lung cancer and non-small cell lung cancer, ovarian cancer, rectal cancer, rhabdomyosarcomasmall intestine cancer, soft tissue sarcoma, squamous cell carcinoma, squamous neck cancer, stomach cancer, uterine cancer, vaginal cancer, and vulvar cancer. In some specific embodiments, the cancer is selected from the group consisting of bladder, breast, colon, colorectal, endometrial, esophageal, gastric, head and neck, lung, and ovarian cancers.

This application also discloses nucleic acids that encode the antibodies or antigen binding fragments thereof, as disclosed herein. This application also discloses vectors comprising the nucleic acids, and host cells comprising the vector or the nucleic acids. The present application also discloses processes for producing an antibody or antigen binding fragment as disclosed herein comprising cultivating the host cell and recovering the antibody from the culture.

In one embodiment, this application discloses diagnostic reagents comprising the antibody or antigen binding fragment thereof as disclosed herein. In some embodiments, the diagnostic reagents comprise the antibody or antigen binding fragment as disclosed herein labeled with a radiolabel, a fluorophore, a chromophore, an imaging agent, or a metal ion.

This application also discloses a process for producing an anti-P-cadherin antibody drug conjugate as disclosed herein, the process comprising:
(a) (i) conjugating a linker L to a drug moiety D as disclosed herein; and
   (ii) conjugating said linker-drug moiety to the antibody recovered from the cell culture disclosed herein;
or
(b) (i) conjugating a linker L to the antibody recovered from the cell culture disclosed herein; and
   (ii) conjugating said linker-antibody conjugate to a drug moiety D as disclosed herein;
and
(c) purifying the antibody drug conjugate.

This application also discloses a process for producing an anti-P-cadherin antibody drug conjugate comprising:
(a) (i) conjugating a linker L as disclosed herein to a drug moiety D as disclosed herein; and
   (ii) conjugating said linker-drug moiety to the antibody as disclosed herein;
or
(b) (i) conjugating a linker L to the antibody as disclosed herein; and
   (ii) conjugating said linker-antibody conjugate to a drug moiety D as disclosed herein;
and
(c) purifying the antibody drug conjugate.

### BRIEF DESCRIPTION OF THE DRAWINGS

FIG. 1 depicts the overall view of the crystal structure of human P-cadherin EC1_EC2, showing the first two cadherin-repeat domains of the extracellular domain of human P-cadherin, with the three calcium binding sites located at the domain-domain junction.

FIG. 2 depicts the overall view of the crystal structure of two P-cadherin antibody Fabs complexed with two human P-cadherin proteins, forming the asymmetric unit of the crystal. The inset is a close-up view of the contact region involving the EC1 domain of the two P-cadherin molecules. There are only a few crystal contacts between the two complexes.

FIG. 3 is a graph depicting human P-cadherin epitope residues that contact residues of the Fab of P-cadherin antibody NOV169N31Q. The amino acid sequence of the human P-cadherin EC1 domain is listed on the horizontal axis. The upper part of the graph shows the number of direct intermolecular contacts between the protein antigen and the antibody, as identified by the program NCONT using a cut-off distance of 4.0 Å between non-hydrogen atoms. The lower part of the graph shows the reduction in solvent-accessible surface (in Å2) incurred by P-cadherin residues upon antibody binding, as calculated by the program AREAIMOL. The β-barrel structure of the EC1 domain is schematically shown as a string of arrows with labels corresponding to the numbering of the β-strands.

FIG. 4 depicts a close-up view of the crystal structure of N-terminal cadherin-repeat (EC1) domain of human P-cadherin (grey cartoon) with all amino acid residues interacting with the antibody (4.0 Å cut-off distance) shown in black stick (antibody view).

FIG. 5 depicts a sequence alignment of the human and cynomolgus (“cyno”; Macaca fascicularis) P-cadherin EC1 domains. Amino acid residues in bold black font are involved in direct intermolecular contacts (<4.0 Å) with the NOV169N31Q antibody. Amino acid residues in bold grey font and indicated with arrows are farther away but experience a reduction of their solvent-accessible surface upon antibody binding. Note that both categories of epitope residues are fully conserved in cynomolgus P-cadherin.

FIG. 6 depicts a multiple sequence alignment of the EC1 domain of human cadherins. Note that P-cadherin is also referred to as “cadherin-3”. Boxed residues are located at the antigen-antibody interface as determined by a reduction of their solvent-accessible surface. Boxed in thick lines is the insertion found in human cadherins 1 through 4. Note that the key epitope residue Glu155 is not conserved in other human cadherins.

FIG. 7 depicts micrographs that illustrate the effect of P-cadherin antibody NOV169N31Q on P-cadherin mediated cellular adhesion. Cells were pre-treated with NOV169N31Q or a non-specific human IgG1antibody prior to induction of spheroid formation. Spheroid shapes and densities were assessed by microscopy after a 132 hr incubation period.

FIG. 8 depicts graphs that illustrate the in vitro cytotoxic potency of ADC NOV169N31Q-KB-22 in P-cadherin positive (HCC70, HCC1954, HCC1806 and SCaBER) cell lines. Graphs depict in vitro dose-response of NOV169N31Q-KB-22 in HCC1954 (P-cadherin+), (B) HCC70 (P-cadherin+), (C) HCC1806 (P-cadherin+), and (D) SCaBER (P-cadherin+) cells. Viability was measured after 5 days of treatment with auristatin (Me-MMAF, square), isotype control ADC (hIgG1-KB-22, triangle), or NOV169N31Q-KB-22 (circle).

FIG. 9 depicts a graph illustrating in vivo efficacy of NOV169N31Q-KB-22 ADC against HCC70 triple negative breast cancer model in mice. Isotype control ADC 3207-KB-22 was dosed at 10 mg/kg (triangle), while NOV169N31Q-KB-22 was dosed at 2.5 mg/kg (open circle) and 0.625 mg/kg (closed circle).

FIG. 10 depicts body weight changes of mice following dosing of NOV169N31Q-KB-22 ADC in HCC70 triple negative breast cancer model.

FIG. 11 depicts a graph illustrating in vivo efficacy of NOV169N31Q-152/375C-77 ADC against HCC70 triple negative breast cancer model in mice. Animals were either untreated (closed circle), treated with 1 mg/kg NOV169N31Q-152/375C-77 (open circle), or treated with 2 mg/kg NOV169N31Q-152/375C-77 (diamond).

FIG. 12 depicts body weight changes of mice following dosing of NOV169N31Q-152/375C-77 ADC in HCC70 triple negative breast cancer model.

### DETAILED DESCRIPTION OF THE INVENTION

### Definitions

Unless stated otherwise, the following terms and phrases as used herein are intended to have the following meanings:

As used herein, the term “a,” “an,” “the” and similar terms used in the context of the present invention (especially in the context of the claims) are to be construed to cover both the singular and plural unless otherwise indicated herein or clearly contradicted by the context.

The term “alkyl” refers to a monovalent saturated hydrocarbon chain having the specified number of carbon atoms. For example, C₁₋₆ alkyl refers to an alkyl group having from 1 to 6 carbon atoms. Alkyl groups may be straight or branched. Representative branched alkyl groups have one, two, or three branches. Examples of alkyl groups include, but are not limited to, methyl, ethyl, propyl (n-propyl and isopropyl), butyl (n-butyl, isobutyl, sec-butyl, and t-butyl), pentyl (n-pentyl, isopentyl, and neopentyl), and hexyl.

The term “antibody” as used herein refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies (including, e.g., anti-Id antibodies to antibodies of the invention). The antibodies can be of any isotype/class (e.g., IgG, IgE, IgM, IgD, IgA and IgY), or subclass (e.g., IgG1, IgG2, IgG3, IgG4, IgA1 and IgA2).

“Complementarity-determining domains” or “complementary-determining regions (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat, Chothia, international ImMunoGeneTics database (IMGT) (on the worldwide web at www.imgt.org/), and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

The term “antigen binding fragment”, as used herein, refers to a polypeptide including one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), camelid antibodies, disulfide-linked Fvs (sdFv), Fab fragments, F(ab′) fragments, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen binding fragment.” These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, single domain antibodies, intrabodies, diabodies, triabodies, tetrabodies, v-NAR and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8:1057-1062, 1995; and U.S. Pat. No. 5,641,870).

The term “monoclonal antibody” or “monoclonal antibody composition” as used herein refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

The term “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000). Also included are antibodies derived from human sequences wherein one or more CDRs has been mutated for affinity maturation or for manufacturing/payload conjugation purposes. See Hybridoma. 1997 August; 16(4):381-9. Rapid development of affinity matured monoclonal antibodies using RIMMS. Kilpatrick K E, Wring S A, Walker D H, Macklin M D, Payne J A, Su J L, Champion B R, Caterson B, McIntyre G D. Department of Molecular Sciences, Glaxo Wellcome, Research Triangle Park, N.C. 27709, USA

The human antibodies of the invention may include amino acid residues not encoded by human sequences (e.g., mutations introduced by random or site-specific mutagenesis in vitro or by somatic mutation in vivo, or a conservative substitution to promote stability or manufacturing).

The term “recognize” as used herein refers to an antibody or antigen binding fragment thereof that finds and interacts (e.g., binds) with its epitope, whether that epitope is linear or conformational. The term “epitope” refers to a site on an antigen to which an antibody or antigen binding fragment of the invention specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Corresponding human germline sequences can be determined, for example, through the publicly available international ImMunoGeneTics database (IMGT) (on the worldwide web at www.imgt.org/) and V-base (on the worldwide web at vbase.mrc-cpe.cam.ac.uk).

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one embodiment, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some embodiments, antibodies or antibody fragments are selected that cross-react with certain desired molecules.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “equilibrium dissociation constant (KD, M)” refers to the dissociation rate constant (kd, time−1) divided by the association rate constant (ka, time−1, M−1). Equilibrium dissociation constants can be measured using any known method in the art. The antibodies of the present invention generally will have an equilibrium dissociation constant of less than about 10⁻⁷ or 10⁻⁸ M, for example, less than about 10⁻⁹ M or 10⁻¹⁰ M, in some embodiments, less than about 10⁻¹¹ M, 10⁻¹² M or 10⁻¹³ M.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

As used herein, the phrase “consisting essentially of” refers to the genera or species of active pharmaceutical agents included in a method or composition, as well as any excipients inactive for the intended purpose of the methods or compositions. In some embodiments, the phrase “consisting essentially of” expressly excludes the inclusion of one or more additional active agents other than an antibody drug conjugate of the invention. In some embodiments, the phrase “consisting essentially of” expressly excludes the inclusion of one or more additional active agents other than an antibody drug conjugate of the invention and a second co-administered agent.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” are used to refer to amino acid modifications that do not significantly affect or alter the binding characteristics of the antibody containing the amino acid sequence.

The term “optimized” as used herein refers to a nucleotide sequence that has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “antibody drug conjugate” or “immunoconjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the antibody drug conjugate. Additionally, the antibody drug conjugate can be provided in the form of a fusion protein that may be expressed from a polynucleotide encoding the immunoconjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “cytotoxin” or “cytotoxic agent” as used herein, refers to any agent that is detrimental to the growth and proliferation of cells and may act to reduce, inhibit, or destroy a cell or malignancy.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “drug moiety” or “payload” as used herein refers to a chemical moiety that is conjugated to an antibody or antigen binding fragment of the invention, and can include any therapeutic or diagnostic agent, for example, an anti-cancer, anti-inflammatory, anti-infective (e.g., anti-fungal, antibacterial, anti-parasitic, anti-viral), or an anesthetic agent. For example, the drug moiety can be an anti-cancer agent, such as a cytotoxin, including, but not limited to, the cytotoxic peptides described herein. The immunoconjugates of the invention comprise one or more cytotoxic peptides described herein as a payload, but may also include one or more other payloads. Other payloads include, for example, a drug moiety or payload that can be an anti-cancer agent, an anti-inflammatory agent, an antifungal agent, an antibacterial agent, an anti-parasitic agent, an anti-viral agent, or an anesthetic agent. In certain embodiments a drug moiety is selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder and a DHFR inhibitor. Methods for attaching each of these to a linker compatible with the antibodies and method of the invention are known in the art. See, e.g., Singh et al., (2009) Therapeutic Antibodies: Methods and Protocols, vol. 525, 445-457. In addition, a payload can be a biophysical probe, a fluorophore, a spin label, an infrared probe, an affinity probe, a chelator, a spectroscopic probe, a radioactive probe, a lipid molecule, a polyethylene glycol, a polymer, a spin label, DNA, RNA, a protein, a peptide, a surface, an antibody, an antibody fragment, a nanoparticle, a quantum dot, a liposome, a PLGA particle, a saccharide or a polysaccharide.

The term “maytansinoid drug moiety” means the substructure of an antibody-drug conjugate that has the structure of a maytansinoid compound. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and maytansinol analogues have been reported. See U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, and Kawai et al (1984) Chem. Pharm. Bull. 3441-3451), each of which are expressly incorporated by reference. Examples of specific maytansinoids useful for conjugation include DM1, DM3 and DM4.

“Tumor” refers to neoplastic cell growth and proliferation, whether malignant or benign, and all pre-cancerous and cancerous cells and tissues.

The term “anti-tumor activity” means a reduction in the rate of tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, MMTV models, and other known models known in the art to investigate anti-tumor activity.

The term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas.

The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “P-cadherin” (also known as Pcad, PCad, or CDH3) refers to the nucleic acid and amino acid sequence of P-cadherin, which have been published in GenBank Accession Nos. NP_001784, NP_001784.2 (amino acid sequence), and NM_001793.4, GenBank Accession Nos. AA14462, NG_009096, and NG_009096.1 (nucleotide sequences). Sequence information for human P-cadherin domains 1-5 are extracellular and are published in GenBank Acession Nos. NM_001793.4 and NP_001784.

“P-cadherin” also refers to proteins and amino acid sequences that over their full length have at least about 90%, 91%, 92%, 93%, 94%, 95%, 96%, 97%, 98%, 99%, or 100% sequence identity with the amino acid sequence of the above GenBank accession Nos. NP_001784, NP_001784.2.

Structurally, a P-cadherin nucleic acid sequence has over its extracellular domain at least about 90%, 91%, 92%, 93%, 94%, 95%, 96%, 97%, 98%, 99%, or 100% sequence identity with the nucleic acid sequence of GenBank accession numbers NM_001793.4, GenBank Accession Nos. AA14462, NG_009096, and NG_009096.1.

As used herein, the terms “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment, “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically, (e.g., stabilization of a discernible symptom), physiologically, (e.g., stabilization of a physical parameter), or both. In yet another embodiment, “treat,” “treating,” or “treatment” refers to preventing or delaying the onset or development or progression of the disease or disorder.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some embodiments, a therapeutically acceptable amount does not induce or cause undesirable side effects. In some embodiments, a therapeutically acceptable amount induces or causes side effects but only those that are acceptable by the healthcare providers in view of a patient's condition. A therapeutically acceptable amount can be determined by first administering a low dose, and then incrementally increasing that dose until the desired effect is achieved. A “prophylactically effective dosage,” and a “therapeutically effective dosage,” of the molecules of the invention can prevent the onset of, or result in a decrease in severity of, respectively, disease symptoms, including symptoms associated with cancer.

The term “co-administer” refers to the presence of two active agents in the blood of an individual. Active agents that are co-administered can be concurrently or sequentially delivered.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, a subject is “in need of” a treatment if such subject would benefit biologically, medically or in quality of life from such treatment.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

In certain embodiments, the modified immunoconjugates of the invention are described according to a “cytotoxic peptide-to-antibody” ratio of, e.g., 1, 2, 3, 4, 5, 6, 7, or 8, or 12 or 16; this ratio corresponds to “y” in Formula A and Formula B. While this ratio has an integer value for a specific conjugate molecule, it is understood that an average value is typically used to describe a sample containing many molecules, due to some degree of inhomogeneity within a sample of an immunoconjugate. The average loading for a sample of an immunoconjugate is referred to herein as the “drug to antibody ratio,” or DAR. In some embodiments, the DAR is between about 1 and about 16, and typically is about 1, 2, 3, 4, 5, 6, 7, or 8. In some embodiments, at least 50% of a sample by weight is compound having the average DAR plus or minus 2, and preferably at least 50% of the sample is a product that contains the average DAR plus or minus 1.5. Preferred embodiments include immunoconjugates wherein the DAR is about 2 to about 8, e.g., about 2, about 3, about 4, about 5, about 6, about 7, or about 8. In these embodiments, a DAR of “about q” means the measured value for DAR is within ±20% of q, or preferably within ±10% of q.

As used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms, unless otherwise stated, e.g., where a specific isomer is identified. Optically active (R)- and (S)-isomers may be prepared using chiral synthons or chiral reagents, or resolved using conventional techniques. If the compound contains a double bond, the substituent may be E or Z configuration. If the compound contains a di-substituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlorotheophyllinate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns Ito XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d⁶-acetone, d⁶-DMSO, as well as solvates with non-enriched solvents.

Compounds of the invention that contain groups capable of acting as donors and/or acceptors for hydrogen bonds may be capable of forming co-crystals with suitable co-crystal formers. These co-crystals may be prepared from compounds of the presente application by known co-crystal forming procedures. Such procedures include grinding, heating, co-subliming, co-melting, or contacting in solution compounds of of the invention with the co-crystal former under crystallization conditions and isolating co-crystals thereby formed. Suitable co-crystal formers include those described in WO 2004/078163. Hence the invention further provides co-crystals comprising a compound as disclosed herein

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the (R)- or (S)-configuration; i.e., for optically active compounds, it is often preferred to use one enantiomer to the substantial exclusion of the other enantiomer. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. “Substantially pure” or “substantially free of other isomers” as used herein means the product contains less than 5%, and preferably less than 2%, of other isomers relative to the amount of the preferred isomer, by weight.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present disclosure, including salts, hydrates and solvates thereof, may inherently or by design form polymorphs.

The term “thiol-maleimide” as used herein refers to a group formed by reaction of a thiol with maleimide, having this general formula:

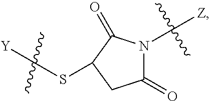

where Y and Z are groups to be connected via the thiol-maleimide linkage and can comprise linker components, antibodies or payloads. The thiol-maleimide linkage can undergo hydrolysis resulting in succinimide ring-opening to give linkages having the following structures:

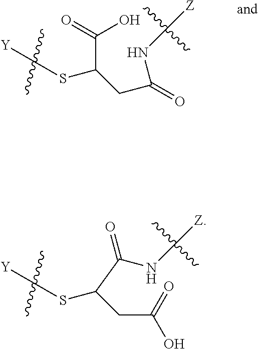

“Pcl” as used herein refers to pyrroline carboxy lysine, e.g.,

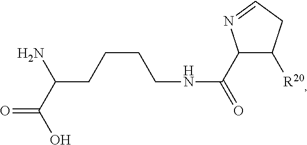

where R²⁰ is H, which has the following formula when incorporated into a peptide:

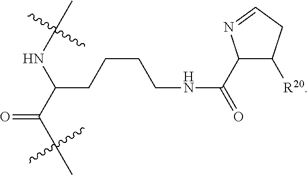

The corresponding compound wherein R²⁰ is methyl is pyrrolysine.

“Cleavable” as used herein refers to a linker or linker component that connects two moieties by covalent connections, but breaks down to sever the covalent connection between the moieties under physiologically relevant conditions, typically a cleavable linker is severed in vivo more rapidly in an intracellular environment than when outside a cell, causing release of the payload to preferentially occur inside a targeted cell. Cleavage may be enzymatic or non-enzymatic, but generally releases a payload from an antibody without degrading the antibody. Cleavage may leave some portion of a linker or linker component attached to the payload, or it may release the payload without any residual part or component of the linker.

“Non-cleavable” as used herein refers to a linker or linker component that is not especially susceptible to breaking down under physiological conditions, e.g., it is at least as stable as the antibody or antigen binding fragment portion of the immunoconjugate. Such linkers are sometimes referred to as “stable”, meaning they are sufficiently resistant to degradation to keep the payload connected to the antigen binding moiety Ab until Ab is itself at least partially degraded, i.e., the degradation of Ab precedes cleavage of the linker in vivo. Degradation of the antibody portion of an ADC having a stable or non-cleavable linker may leave some or all of the linker, and one or more amino acid groups from an antibody, attached to the payload or drug moiety that is delivered in vivo.

The terms “C₁-C₃alkyl”, “C₂-C₃alkyl”, “C₁-C₄alkyl”, “C₁-C₅alkyl”, “C₁-C₆alkyl” and “C₂-C₆alkyl”, as used herein, refer to a fully saturated branched or straight chain hydrocarbon containing 1-3 carbon atoms, 2-3 carbon atoms, 1-4 carbon atoms, 1-5 carbon atoms, 1-6 carbon atoms or 2-6 carbon atoms, respectively. Non-limiting examples of “C₁-C₃alkyl” groups include methyl, ethyl, n-propyl and isopropyl. Non-limiting examples of “C₂-C₃alkyl” groups include ethyl, n-propyl and isopropyl. Non-limiting examples of “C₁-C₄alkyl” groups include methyl, ethyl, n-propyl, isopropyl, n-butyl, isobutyl, sec-butyl and tert-butyl. Non-limiting examples of “C₁-C₅alkyl” groups include methyl, ethyl, n-propyl, isopropyl, n-butyl, isobutyl, sec-butyl, tert-butyl, n-pentyl and isopentyl. Non-limiting examples of “C₁-C₆alkyl” groups include methyl, ethyl, n-propyl, isopropyl, n-butyl, isobutyl, sec-butyl, tert-butyl, n-pentyl, isopentyl and hexyl. Non-limiting examples of “C₂-C₆alkyl” groups include ethyl, n-propyl, isopropyl, n-butyl, isobutyl, sec-butyl, tert-butyl, n-pentyl, isopentyl and hexyl.

As used herein, the term “alkylene” refers to a divalent alkyl group having 1 to 10 carbon atoms, and two open valences to attach to other features. Unless otherwise provided, alkylene refers to moieties having 1 to 10 carbon atoms, 1 to 6 carbon atoms, or 1 to 4 carbon atoms. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like.

The terms “C₁-C₃alkoxy”, “C₂-C₃alkoxy”, “C₁-C₄alkoxy”, “C₁-C₅alkoxy”, “C₁-C₆alkoxy” and “C₂-C₆alkoxy, as used herein, refer to the groups —O—C₁-C₃alkyl, —O—C₂-C₃alkyl, —O—C₁-C₄alkyl, —O—C₁-C₅alkyl, —O—C₁-C₆alkyl and —O—C₂-C₆alkyl, respectively, wherein the groups “C₁-C₃alkyl”, “C₂-C₃alkyl^{”}, “C₁-C₄alkyl”, “C₁-C₅alkyl”, “C₁-C₆alkyl” and “C₂-C₆alkyl” are as defined herein. Non-limiting examples of “C₁-C₃alkoxy” groups include methoxy, ethoxy, n-propoxy and isopropoxy. Non-limiting examples of “C₂-C₃alkoxy” groups include ethoxy, n-propoxy and isopropoxy. Non-limiting examples of “C₁-C₄alkoxy” groups include methoxy, ethoxy, n-propoxy, isopropoxy, n-butoxy, isobutoxy, sec-butoxy and tert-butoxy. Non-limiting examples of “C₁-C₅alkoxy” groups include methoxy, ethoxy, n-propoxy, isopropoxy, n-butoxy, isobutoxy, sec-butoxy, tert-butoxy, n-pentyloxy and isopentyloxy. Non-limiting examples of “C₁-C₆alkoxy” groups include methoxy, ethoxy, n-propoxy, isopropoxy, n-butoxy, isobutoxy, sec-butoxy, tert-butoxy, n-pentyloxy, isopentyloxy and hexyloxy. Non-limiting examples of “C₂-C₆alkoxy” groups include ethoxy, n-propoxy, isopropoxy, n-butoxy, isobutoxy, sec-butoxy, tert-butoxy, n-pentyloxy, isopentyloxy and hexyloxy.

As used herein, the term “halogen” (or halo) refers to fluorine, bromine, chlorine or iodine, in particular fluorine or chlorine. Halogen-substituted groups and moieties, such as alkyl substituted by halogen (haloalkyl) can be mono-, poly- or per-halogenated.

As used herein, the term “heteroatoms” refers to nitrogen (N), oxygen (O) or sulfur (S) atoms, in particular nitrogen or oxygen, unless otherwise provided.

The term “4-8 membered heterocycloalkyl,” as used herein refers to a saturated 4-8 membered monocyclic hydrocarbon ring structure wherein one to two of the ring carbons of the hydrocarbon ring structure are replaced by one to two NR groups, wherein R is hydrogen, a bond, an R⁵ group as defined herein or an R⁷ group as defined herein. Non-limiting examples of 4-8 membered heterocycloalkyl groups, as used herein, include azetadinyl, azetadin-1-yl, azetadin-2-yl, azetadin-3-yl, pyrrolidinyl, pyrrolidin-1-yl, pyrrolidin-2-yl, pyrrolidin-3-yl, pyrrolidin-4-yl, pyrrolidin-5-yl, piperidinyl, piperidin-1-yl, piperidin-2-yl, piperidin-3-yl, piperidin-4-yl, piperidin-5-yl, piperidin-6-yl, piperazinyl, piperazin-1-yl, piperazin-2-yl, piperazin-3-yl, piperazin-4-yl, piperazin-5-yl, piperazin-6-yl, azepanyl, azepan-1-yl, azepan-2-yl, azepan-3-yl, azepan-4-yl, azepan-5-yl, azepan-6-yl, and azepan-7-yl.

The term “6 membered heterocycloalkyl,” as used herein refers to a saturated 6 membered monocyclic hydrocarbon ring structure wherein one to two of the ring carbons of the hydrocarbon ring structure are replaced by one to two NR groups, wherein R is hydrogen, a bond, an R⁵ group as defined herein or an R⁷ group as defined herein. Non-limiting examples of 6 membered heterocycloalkyl groups, as used herein, include piperidinyl, piperidin-1-yl, piperidin-2-yl, piperidin-3-yl, piperidin-4-yl, piperidin-5-yl, piperidin-6-yl, piperazinyl, piperazin-1-yl, piperazin-2-yl, piperazin-3-yl, piperazin-4-yl, piperazin-5-yl and piperazin-6-yl.

The term “5-8 membered fused bicyclic heterocycloalkyl,” as used herein refers to a saturated 5-8 membered fused bicyclic hydrocarbon ring structure, wherein one to two of the ring carbons of the hydrocarbon ring structure are replaced by one to two NR groups, R is hydrogen, a bond, an R⁵ group as defined herein or an R⁷ group as defined herein. Non-limiting examples of 5-8 membered fused bicyclic heterocycloalkyl groups, as used herein, include 3-azabicyclo[3.1.0]hexanyl and 3-azabicyclo[4.1.0]heptanyl.

The immunoconjugate naming convention used herein is “Antibody-Compound Number” or “Antibody-Conjugation Method-Compound Number”, where Compound Number refers to the compound used for conjugation to the particular antibody, and Conjugation Method refers to either “KB” for ketone bridge conjugation between an antibody and a linker-payload compound or payload compound, or “Cys”, “152/375C” or “107/360C” for conjugation between cysteine resides of the antibody, particularly modified cysteine residues in the constant regions of the heavy chain, light chain, or both heavy and light chains of the antibody, and the linker-payload compound or payload compound.

The present invention provides antibodies, antibody fragments (e.g., antigen binding fragments), and drug conjugates thereof, i.e. antibody drug conjugates or ADCs, that bind to P-cadherin. In particular, the present invention provides antibodies and antibody fragments (e.g., antigen binding fragments) that bind to P-cadherin, and internalize upon such binding. The antibodies and antibody fragments (e.g., antigen binding fragments) of the present invention can be used for producing antibody drug conjugates. Furthermore, the present invention provides antibody drug conjugates that have desirable pharmacokinetic characteristics and other desirable attributes, and thus can be used for treating cancer expressing P-cadherin. The present invention further provides pharmaceutical compositions comprising the antibody drug conjugates of the invention, and methods of making and using such pharmaceutical compositions for the treatment of cancer.

### Antibody Drug Conjugates

The present invention provides antibody drug conjugates also referred to as immunoconjugates, where an antibody, antigen binding fragment or its functional equivalent that specifically binds to P-cadherin is linked to a drug moiety. In one aspect, the antibodies, antigen binding fragments or their functional equivalents of the invention are linked, via covalent attachment by a linker, to a drug moiety that is an anti-cancer agent. The antibody drug conjugates of the invention can selectively deliver an effective dose of an anti-cancer agent (e.g., a cytotoxic agent) to tumor tissues expressing P-cadherin, whereby greater selectivity (and lower efficacious dose) may be achieved.

In some embodiments of the invention, the drug moiety comprises a cytotoxic peptide, wherein the cytotoxic peptides comprise auristatin analogs.

In one aspect, the invention provides an antibody drug conjugate comprising a formula selected from:

(Formula A) or ((D)_{z})-L)_{y}-Ab (Formula B)

Wherein Ab represents P-cadherin binding antibody described herein;
L is a linker;
D is a drug moiety;
z is an integer from 1 to 8; and
y is an integer from 1-20. In one embodiment, y is an integer from 1 to 10, 2 to 8, or 2 to 5. In a specific embodiment, y is 2, 3, or 4. In some embodiments, z is 1; in other embodiments y is 2, 3 or 4.

While the drug to antibody ratio has an exact value for a specific conjugate molecule (e.g., y multiplied by z in Formula A or Formula B), it is understood that the value will often be an average value when used to describe a sample containing many molecules, due to some degree of inhomogeneity, typically associated with the conjugation step. The average loading for a sample of an immunoconjugate is referred to herein as the drug to antibody ratio, or “DAR.” In some embodiments, the DAR is between about 2 and about 6, and typically is about 3, 3.5, 4, 4.5, 5, 5.5, 6, 6.5, 7.0, 7.5. 8.0. In some embodiments, at least 50% of a sample by weight is compound having the average DAR plus or minus 2, and preferably at least 50% of the sample is a conjugate that contains the average DAR plus or minus 1. Embodiments include immunoconjugates wherein the DAR is about 3.5, 3.6, 3.7, 3.8 or 3.9. In some embodiments, a DAR of ‘about y’ means the measured value for DAR is within 20% of y.

The present invention is also directed to immunoconjugates comprising the antibodies, antibody fragments (e.g., antigen binding fragments) and their functional equivalents as disclosed herein, linked or conjugated to a drug moiety.

In one embodiment, wherein when the antibody drug conjugate comprises Formula A, the drug moiety D is

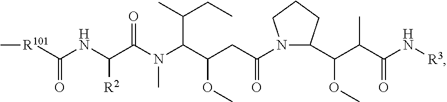

wherein
R¹⁰¹ is a 6 membered heterocycloalkyl divalent radical containing 1-2 N heteroatoms and a C₁-C₂alkylene bridge, wherein the 6 membered heterocycloalkyl divalent radical is C-linked to the

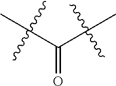 [SMILES: *C(*)=O]

group and is N-linked to L or is C-linked to L, and the 6 membered heterocycloalkyl divalent radical is unsubstituted or substituted with 1 to 3 substituents independently selected from R⁵ and R⁶;
or R¹⁰¹ is a 5-8 membered fused bicyclic heterocycloalkyl divalent radical containing 1-2 N heteroatoms, wherein the 5-8 membered fused bicyclic heterocycloalkyl divalent radical is C-linked to the

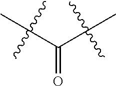 [SMILES: *C(*)=O]

group and is N-linked to L or is C-linked to L, and the 5-8 membered fused bicyclic heterocycloalkyl divalent radical is unsubstituted or substituted with 1 to 3 substituents independently selected from R⁵ and R⁶;
R² is —C₁-C₆alkyl;
R³ is

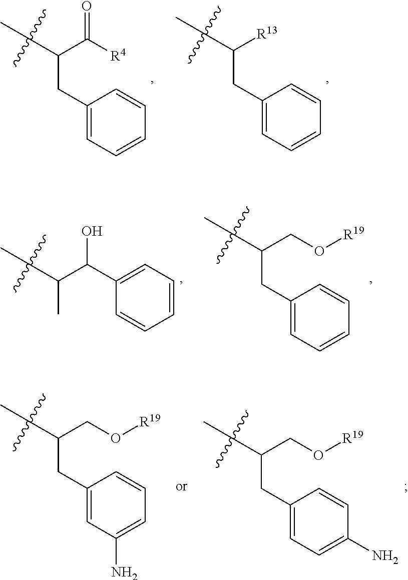

R⁴ is —OH, C₁-C₆alkoxy, —N(R¹⁴)₂, —R¹⁶, —NR¹²(CH₂)ₘN(R¹⁴)₂, or —NR¹²(CH₂)ₘR¹⁶, —NHS(O)₂R₁₁ or

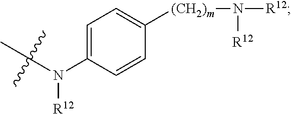

R⁵ is C₁-C₆alkyl, —C(═O)R¹¹, —(CH₂)ₘOH, —C(═O)(CH₂)ₘOH, —C(═O)((CH₂)ₘO)ₙR¹², —((CH₂)ₘO)R¹², or C₁-C₆alkyl which is optionally substituted with —CN, —C(═O)NH₂ or 1 to 5 hydroxyl,
R⁶ is halo, oxo, OH, C₁-C₆alkyl, —N(R¹⁴)₂, —R¹⁶ and —NR¹²C(═O)R¹¹;
R¹¹ is C₁-C₆alkyl or C₁-C₆alkyl which is optionally substituted with 1 to 5 hydroxyl;
each R¹² is independently selected from H and C₁-C₆alkyl;
R¹³ is tetrazolyl, imidazolyl substituted with phenyl, oxadiazolyl substituted with phenyl, pyrazolyl, pyrimidinyl,

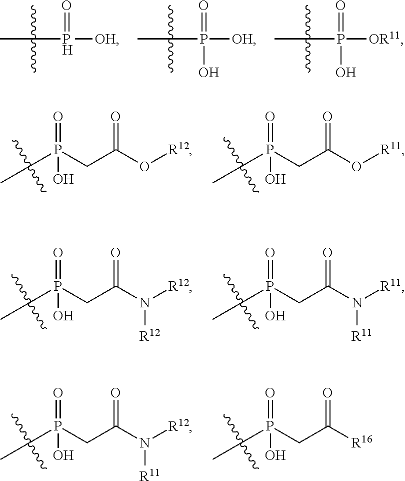

or —CH₂S(═O)₂NH₂;
each R¹⁴ is independently selected from H and C₁-C₆alkyl;
R¹⁶ is an N-linked 4-8 membered heterocycloalkyl containing 1-2 heteroatoms independently selected from N and O;
R¹⁹ is H or C₁-C₆alkyl;
each m is independently selected from 1, 2, 3, 4, 5, 6, 7, 8, 9 and 10,
and
each n is independently selected from 1, 2, 3, 4, 5, 6, 7, 8, 9, 10, 11, 12, 13, 14, 15, 16,17 and 18.

In another embodiment, wherein the antibody drug conjugate comprises Formula B, D is

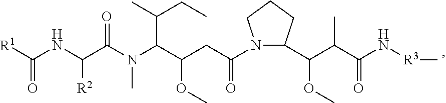

wherein
R¹ is a 6 membered heterocycloalkyl containing 1-2 N heteroatoms and a C₁-C₂alkylene bridge, wherein the 6 membered heterocycloalkyl is unsubstituted or substituted with 1 to 3 substituents independently selected from R⁵ and R⁶;
or R¹ is a 5-8 membered fused bicyclic heterocycloalkyl containing 1-2 N heteroatoms, wherein the 5-8 membered fused bicyclic heterocycloalkyl is unsubstituted or substituted with 1 to 3 substituents independently selected from R⁵ and R⁶;
R² is —C₁-C₆alkyl;
R³ is

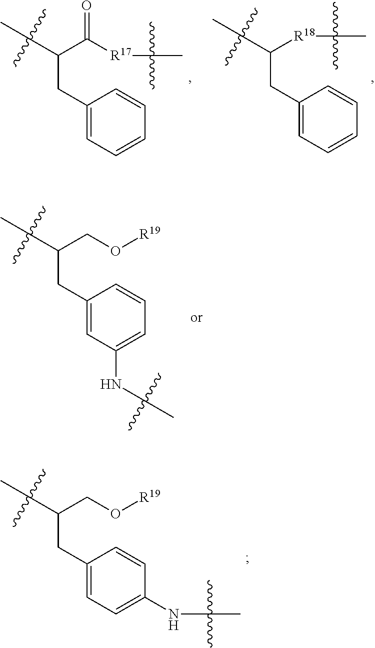

R⁵ is C₁-C₆alkyl, C₁-C₆alkyl which is optionally substituted with 1 to 5 hydroxyl, —C(═O)R¹¹, —(CH₂)ₘOH, —C(═O)(CH₂)ₘOH, —C(═O)((CH₂)ₘO)ₙR¹², or —((CH₂)ₘO)R¹²;
R⁶ is halo, oxo, OH, C₁-C₆alkyl, —N(R¹⁴)₂, —R¹⁶ and —NR¹²C(═O)R¹¹;
R¹¹ is C₁-C₆alkyl C₆alkyl or C₁-C₆alkyl which is optionally substituted with 1 to 5 hydroxyl;
each R¹² is independently selected from H and C₁-C₆alkyl;
each R¹⁴ is independently selected from H and C₁-C₆alkyl;
R¹⁶ is an N-linked 4-8 membered heterocycloalkyl containing 1-2 heteroatoms independently selected from N and O;
R¹⁷ is a bond, —NH—, —NHS(═O)₂—,

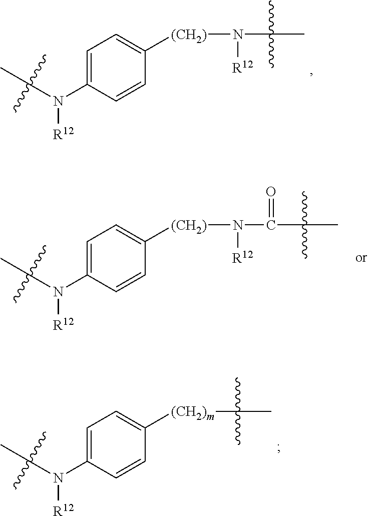

R¹⁸ is a bond,

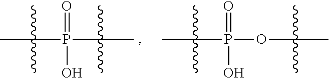 [SMILES: *P(*)(=O)O ; *OP(*)(=O)O]

or —CH₂S(═O)₂NH—;
each m is independently selected from 1, 2, 3, 4, 5, 6, 7, 8, 9 and 10,
and
each n is independently selected from 1, 2, 3, 4, 5, 6, 7, 8, 9, 10, 11, 12, 13, 14, 15, 16,17 and 18.

In one embodiment, the antibody drug conjugate of the present invention is represented by any one of the following structural formulae:

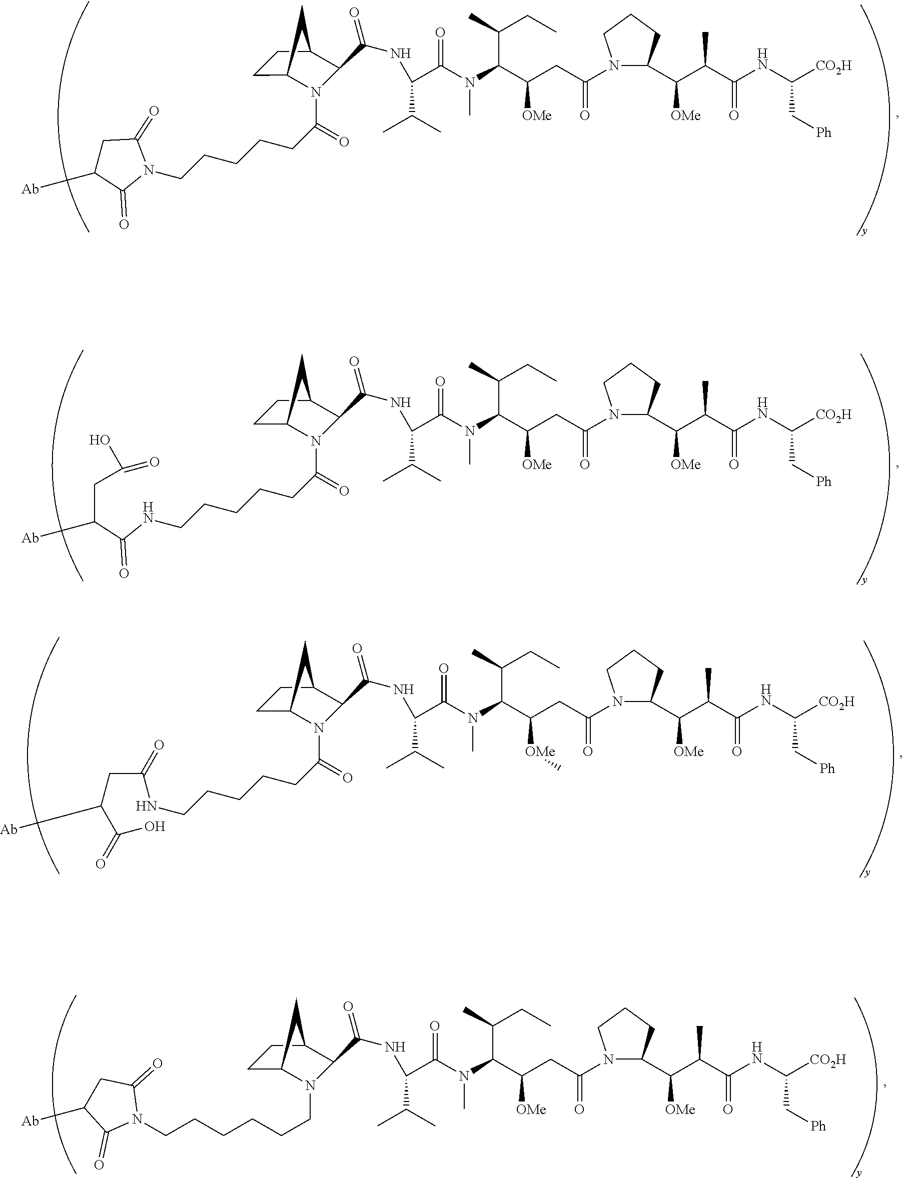

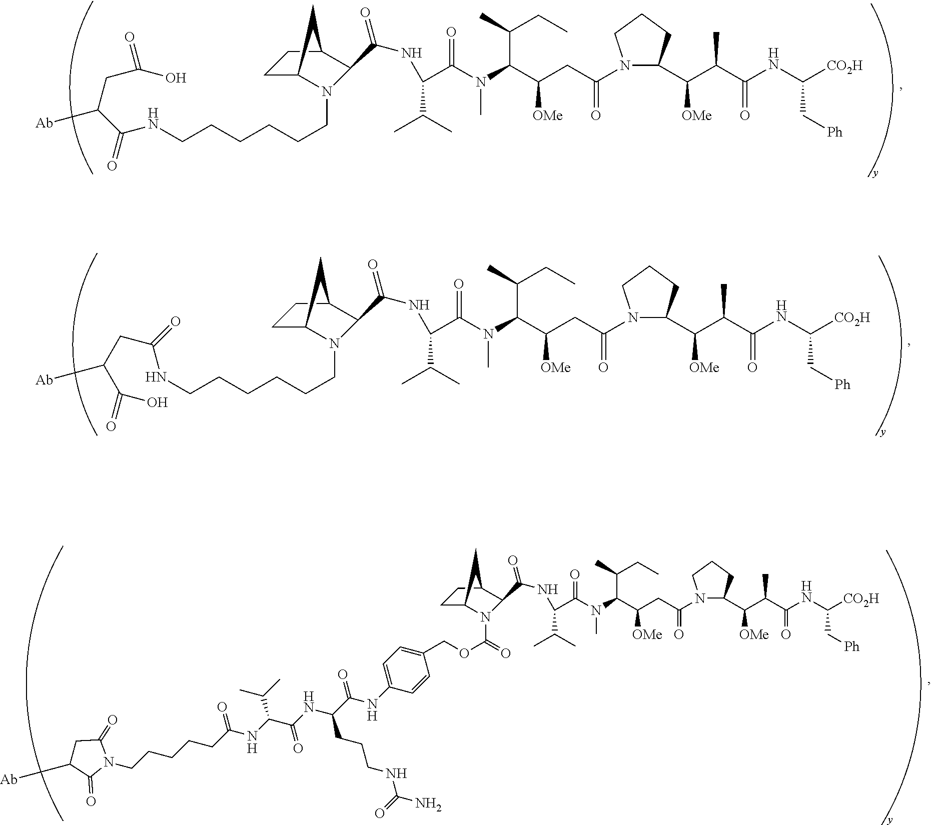

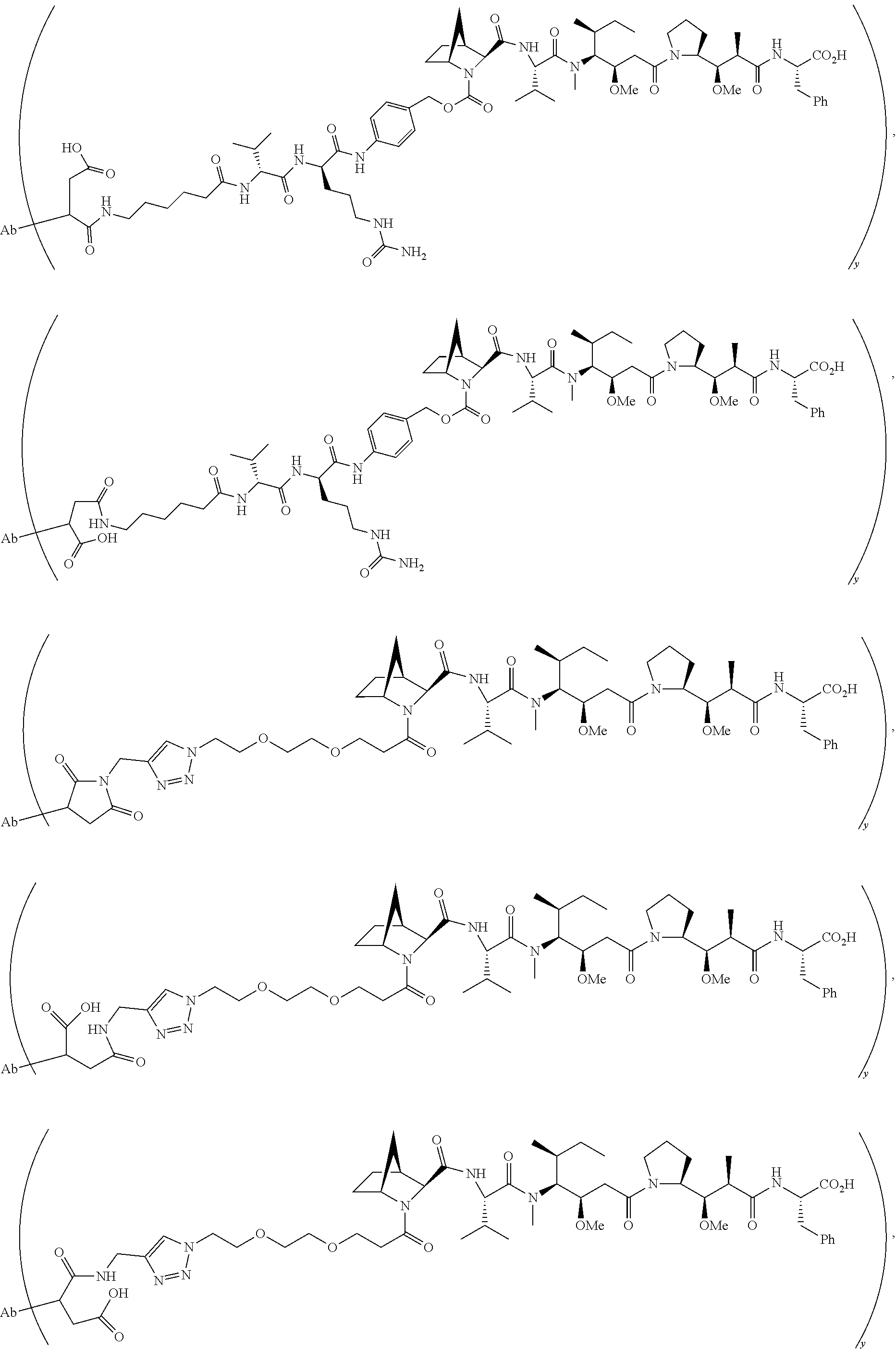

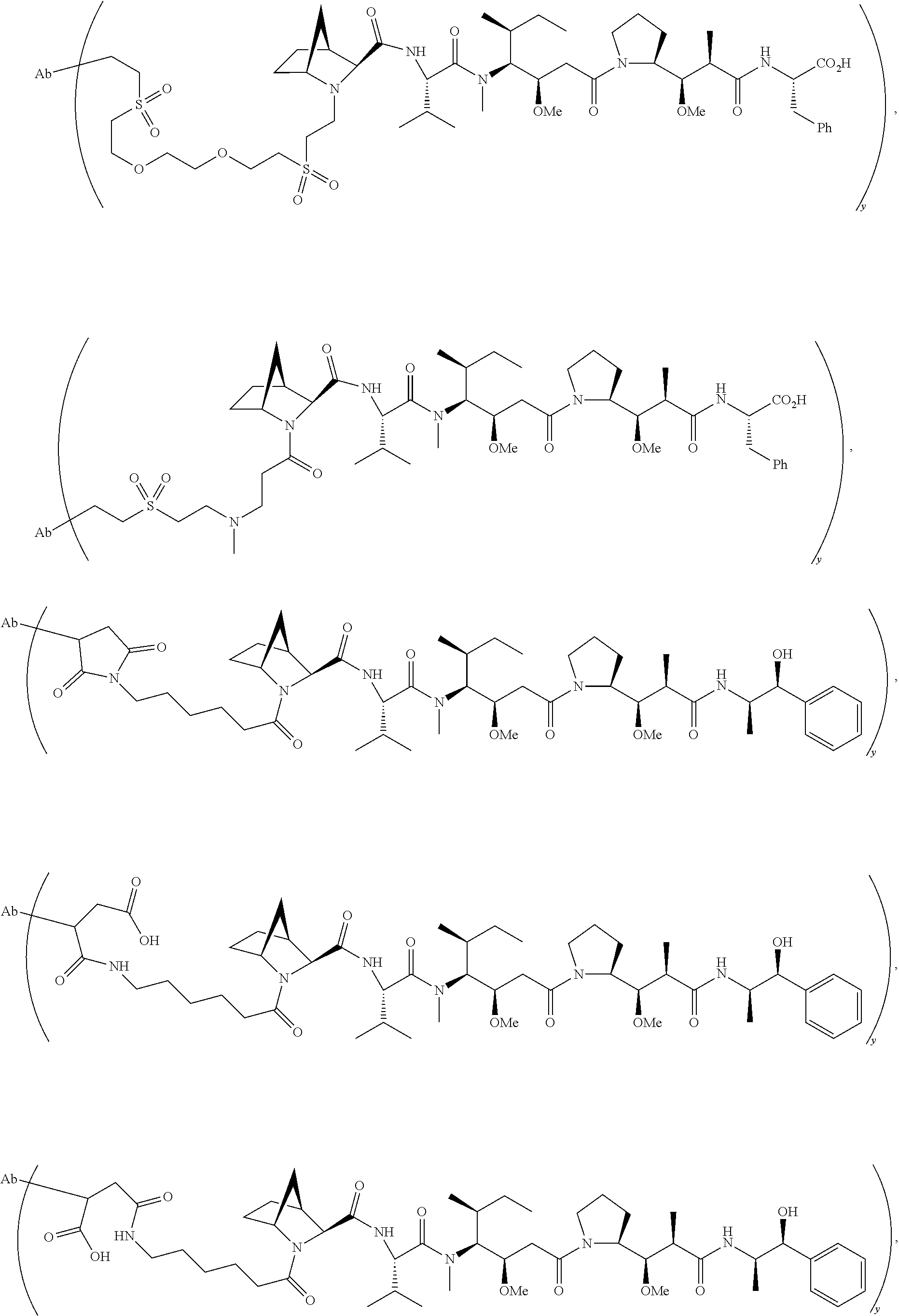

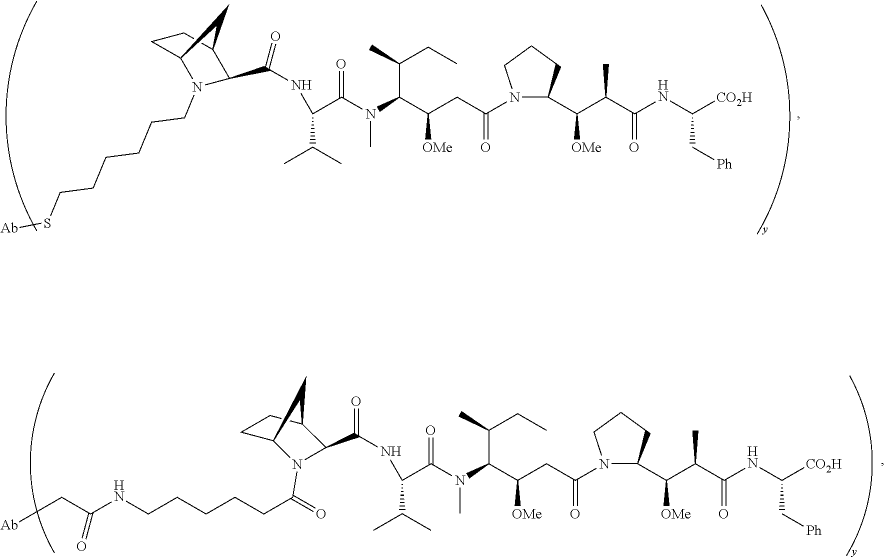

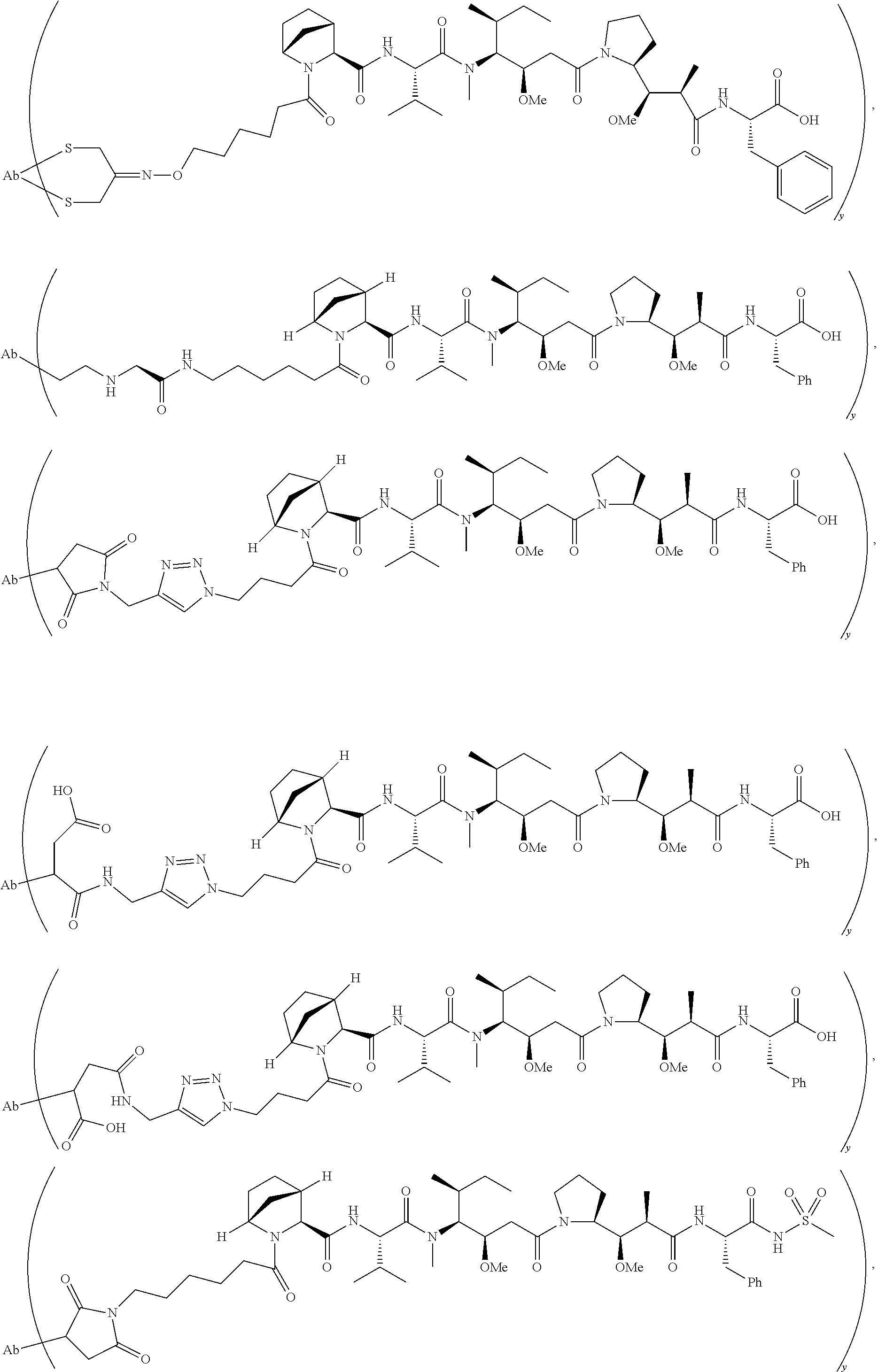

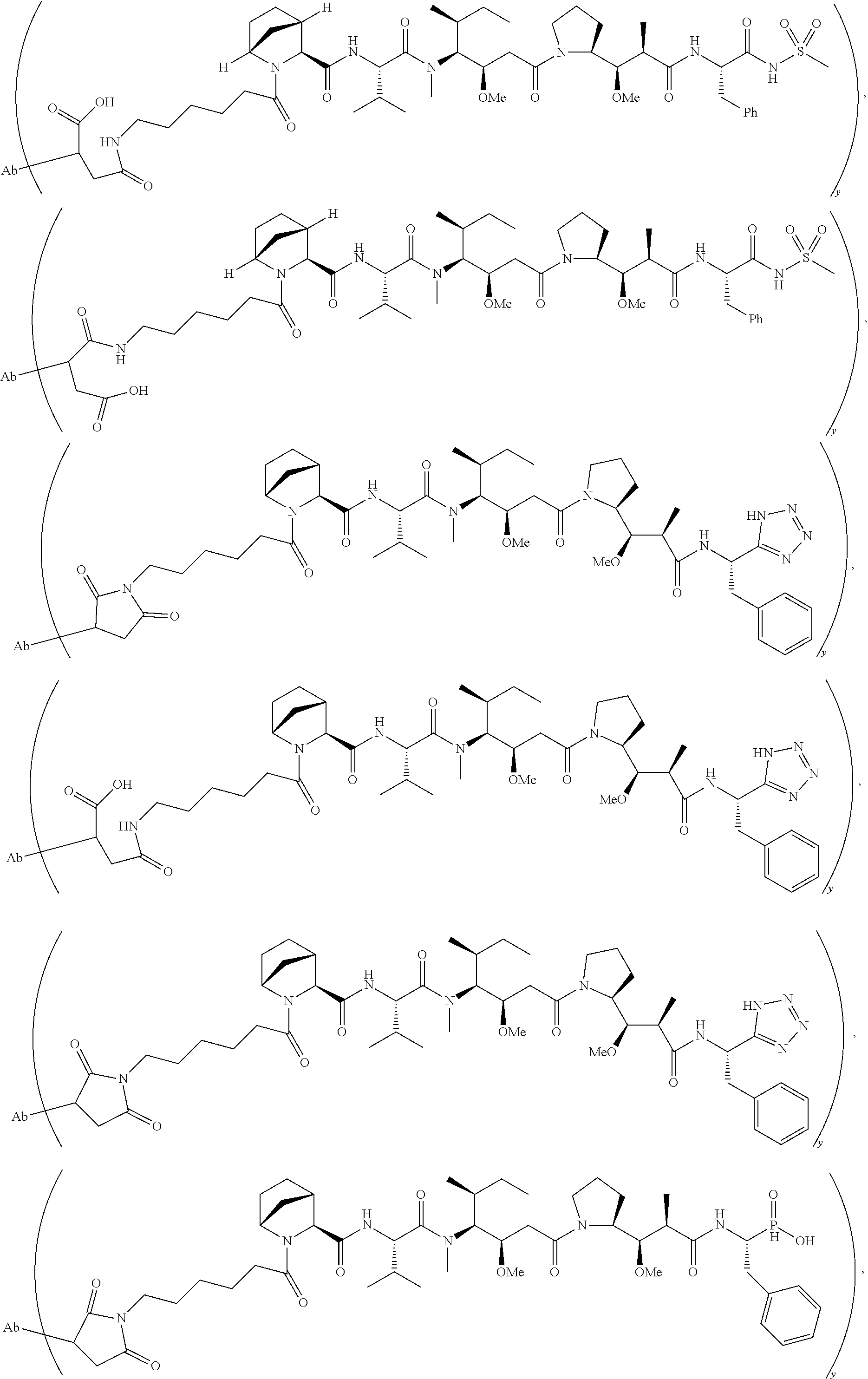

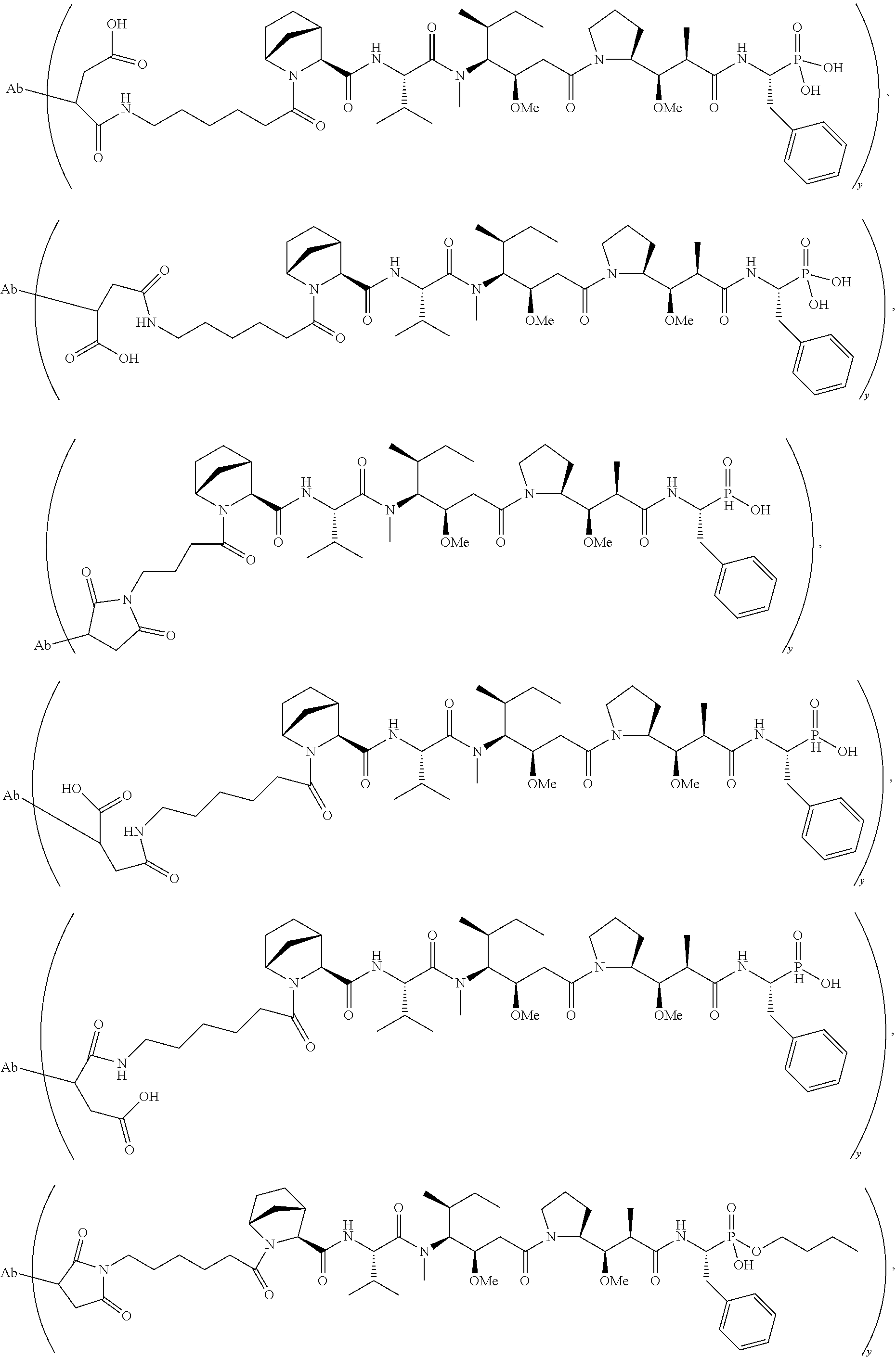

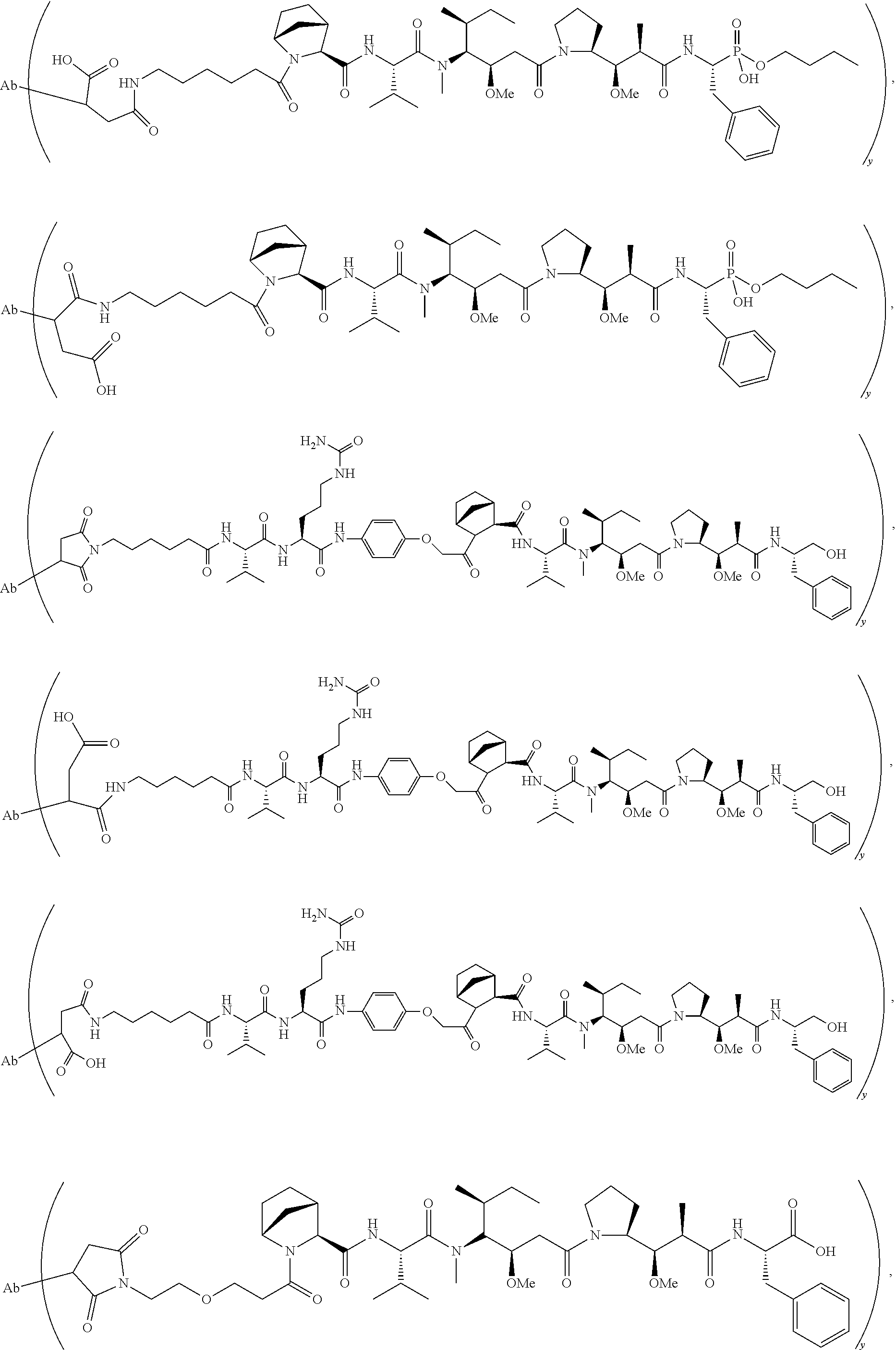

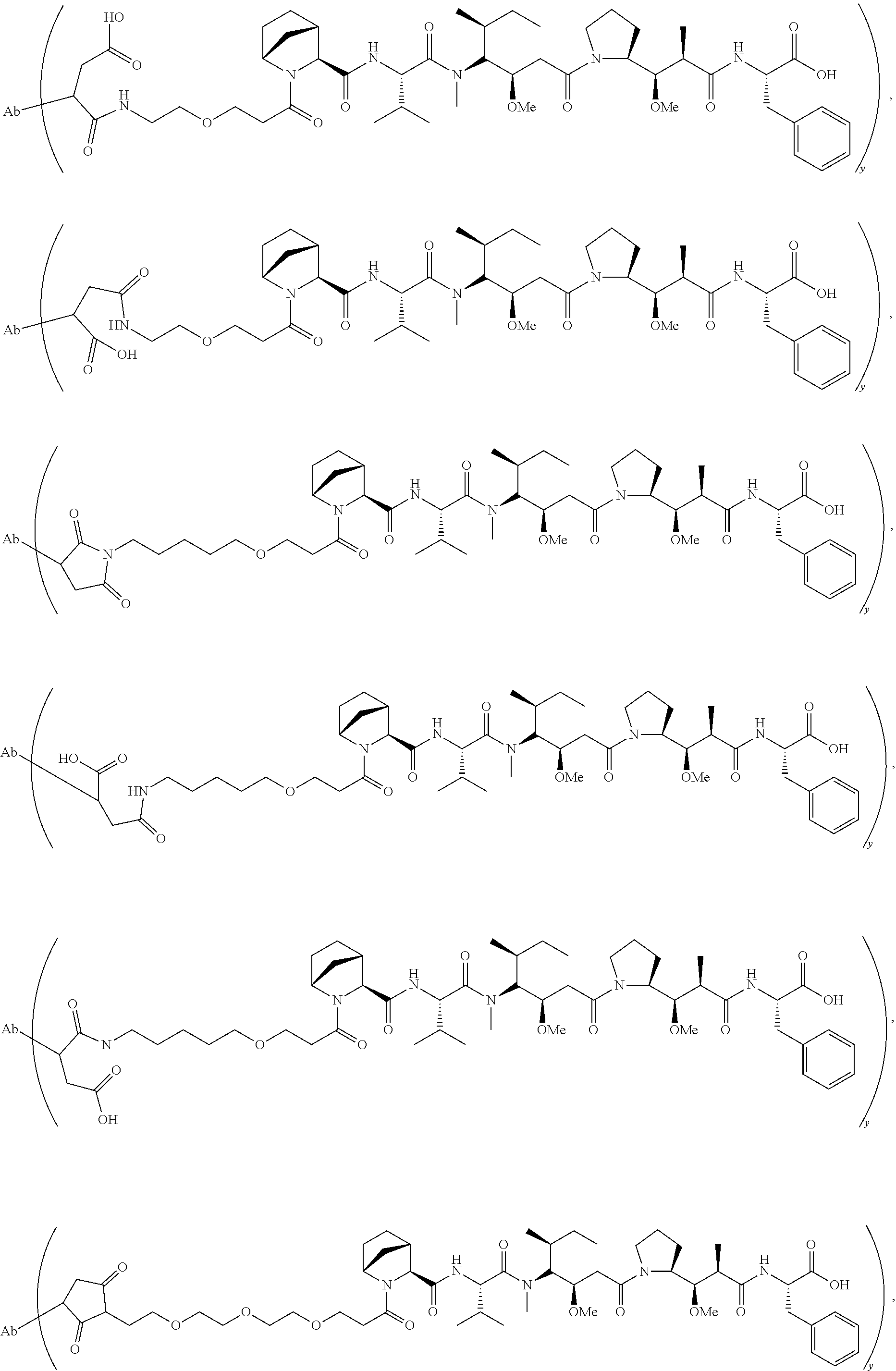

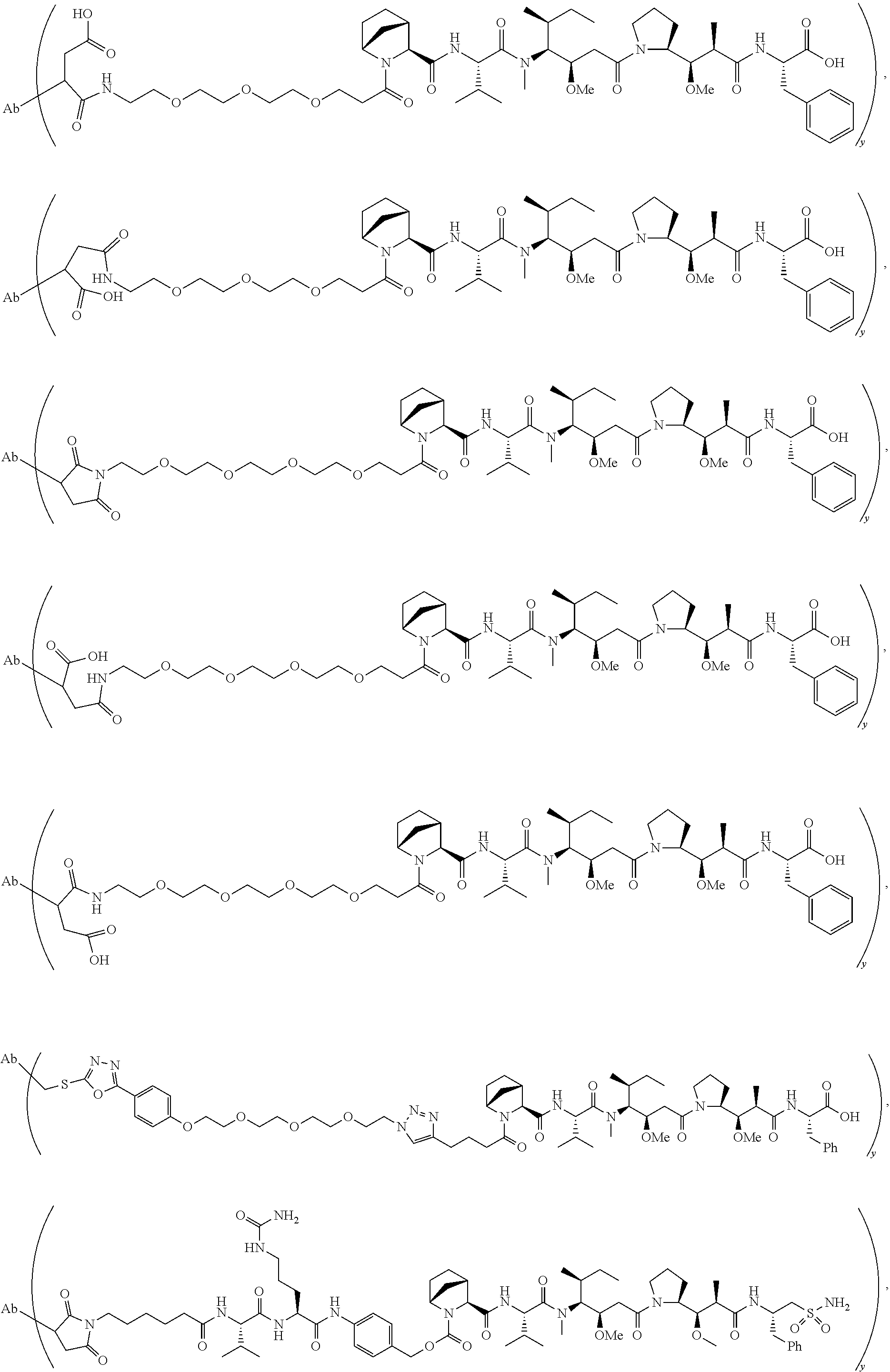

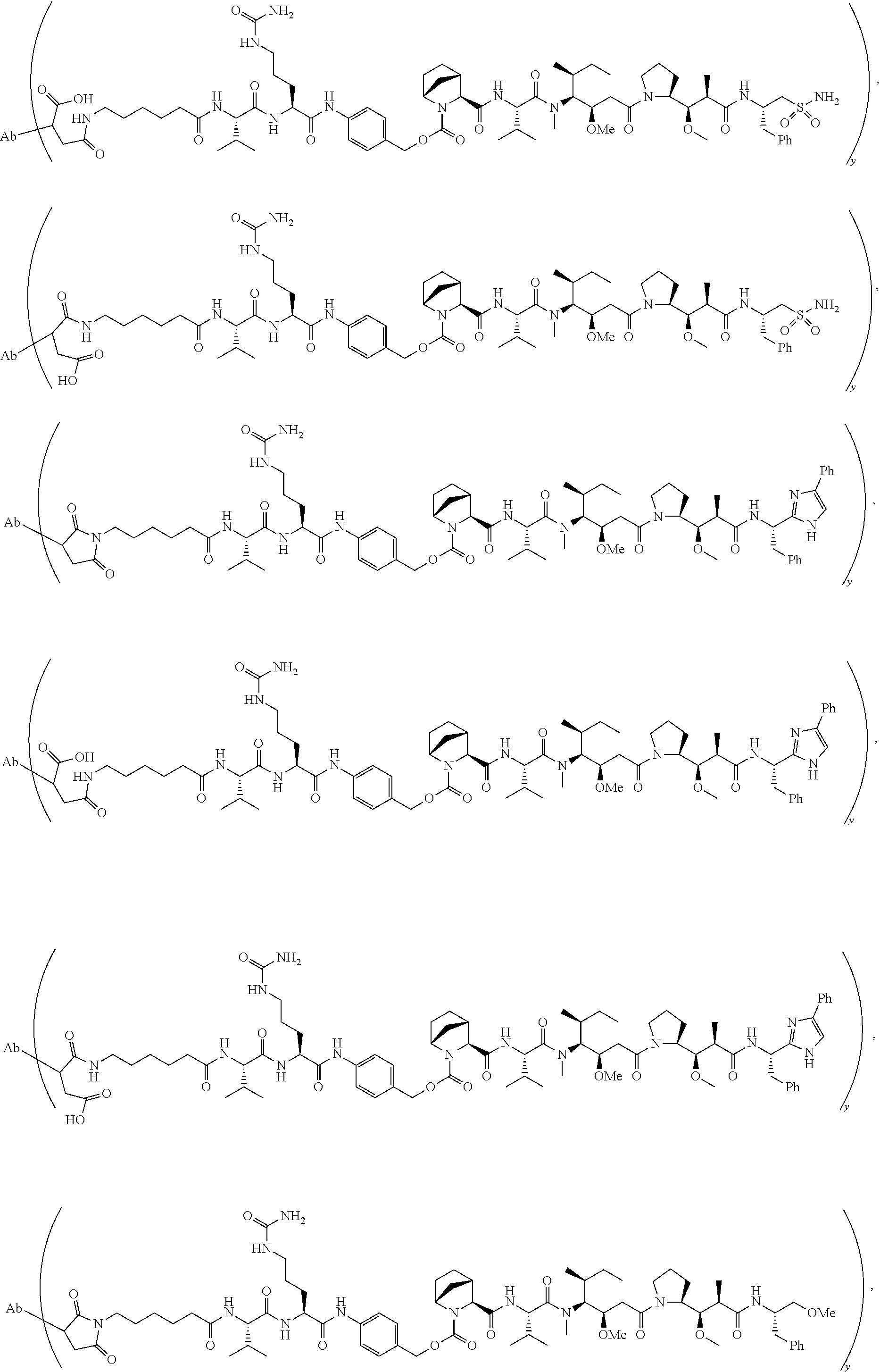

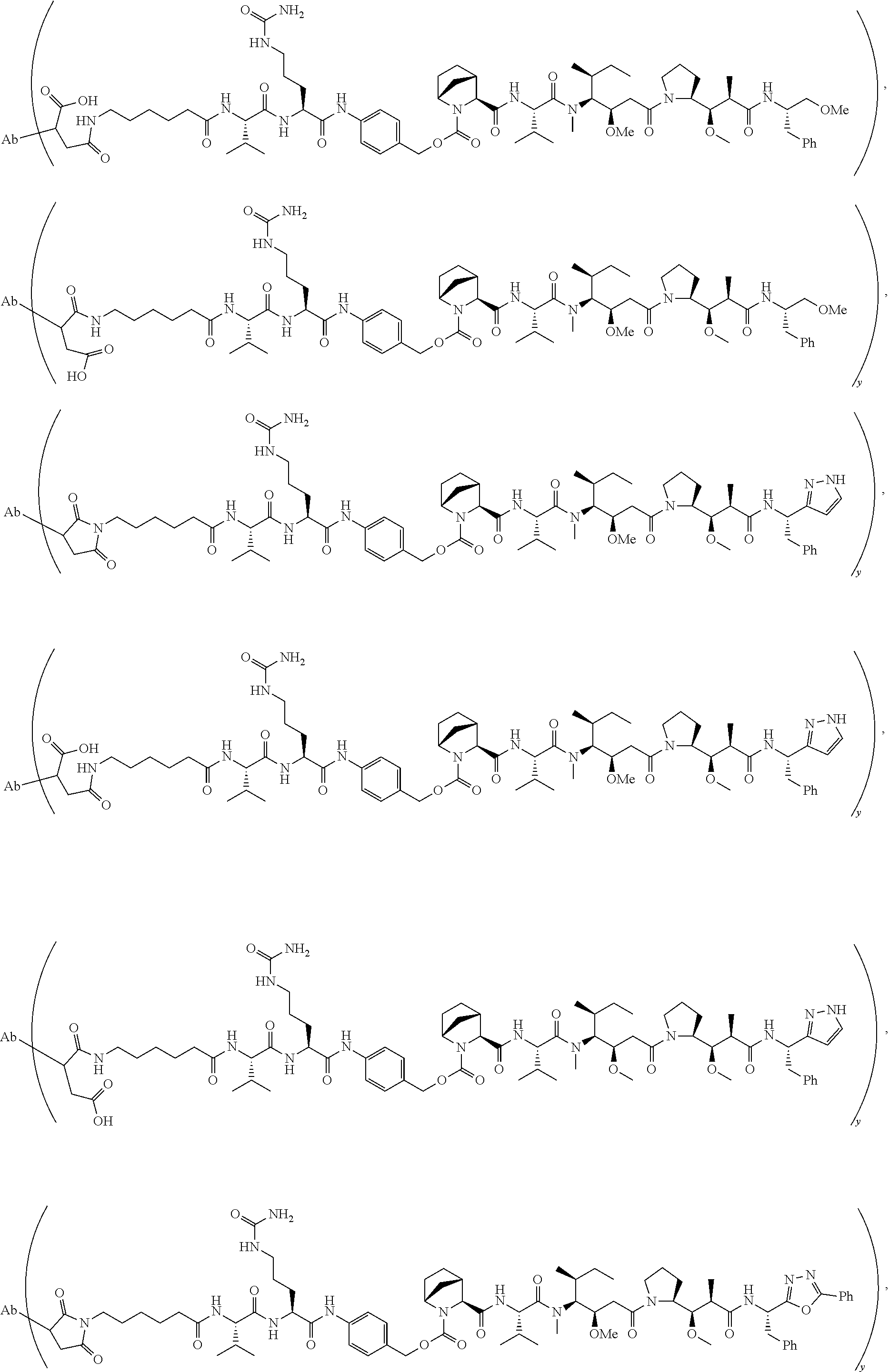

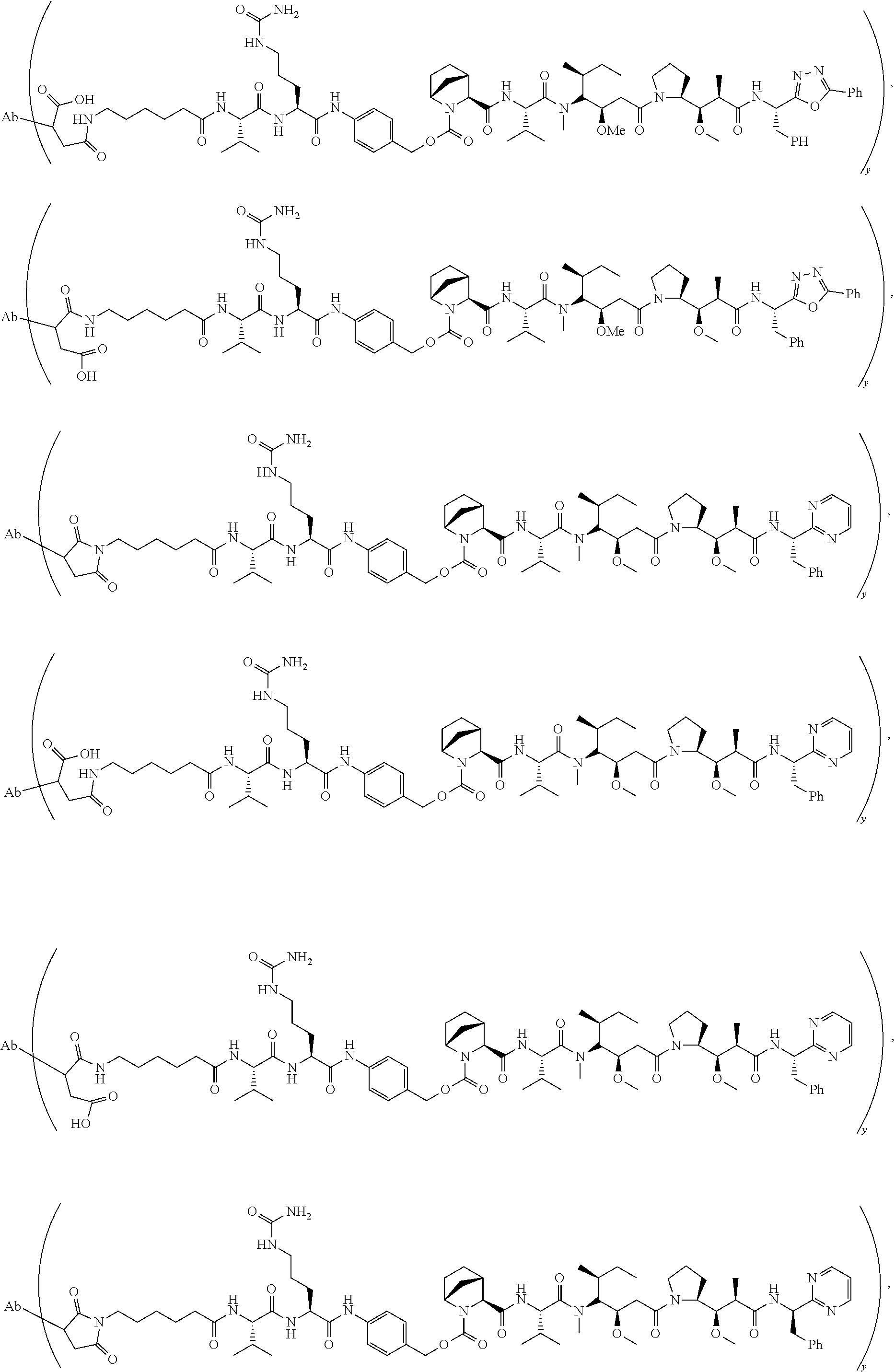

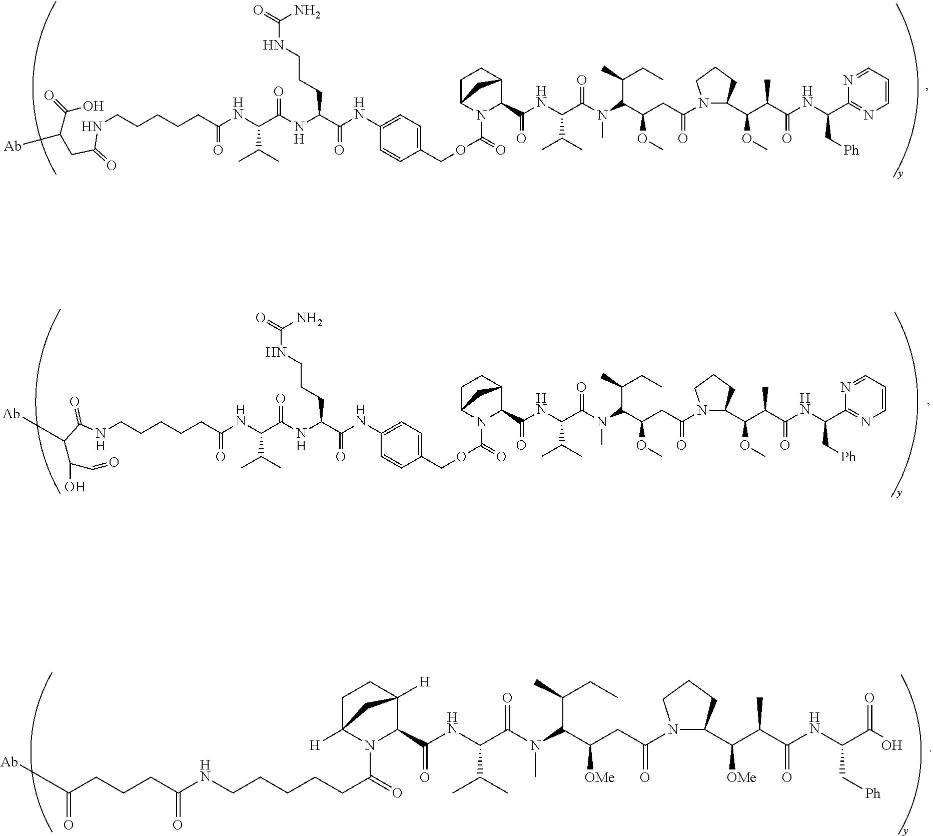

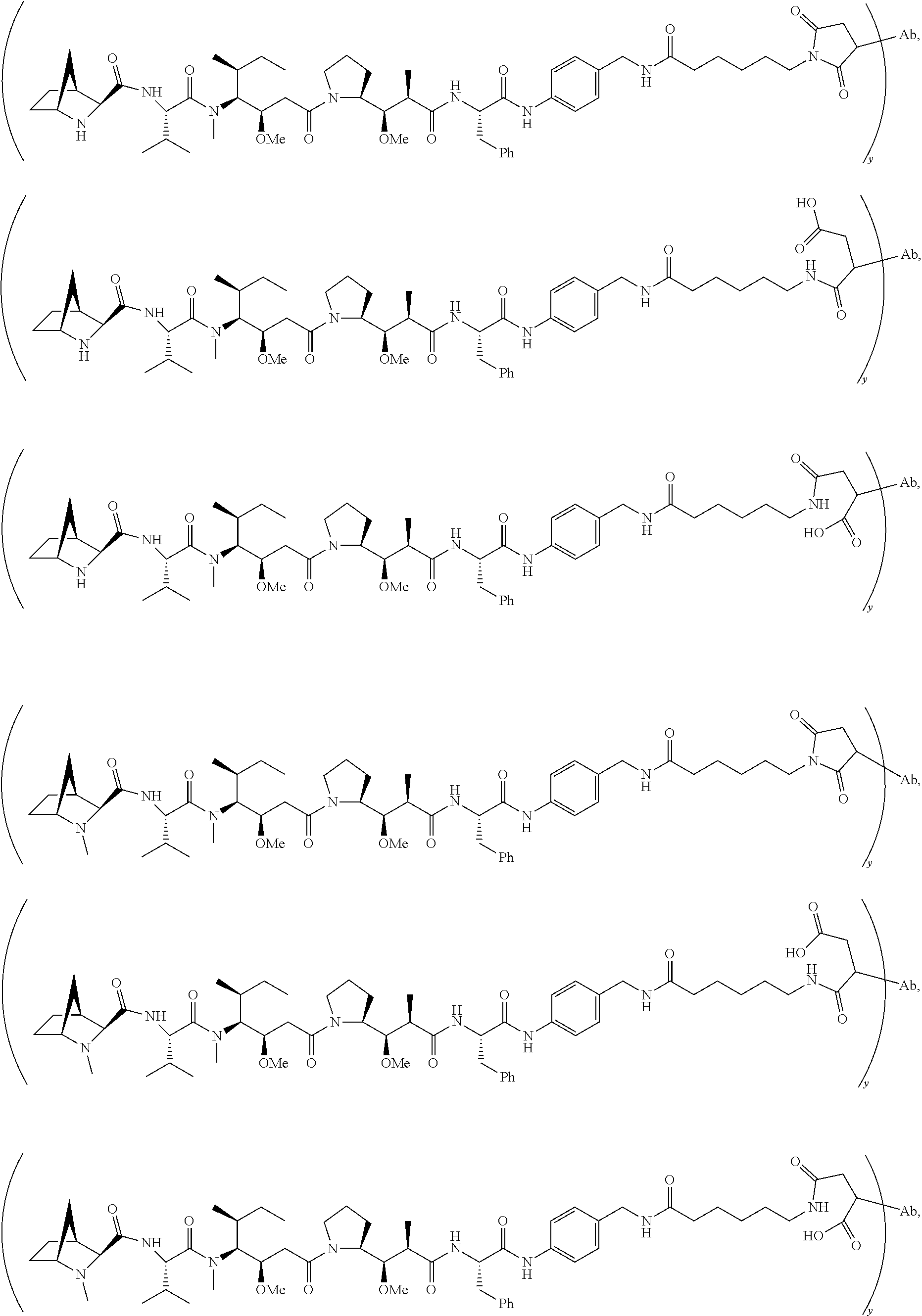

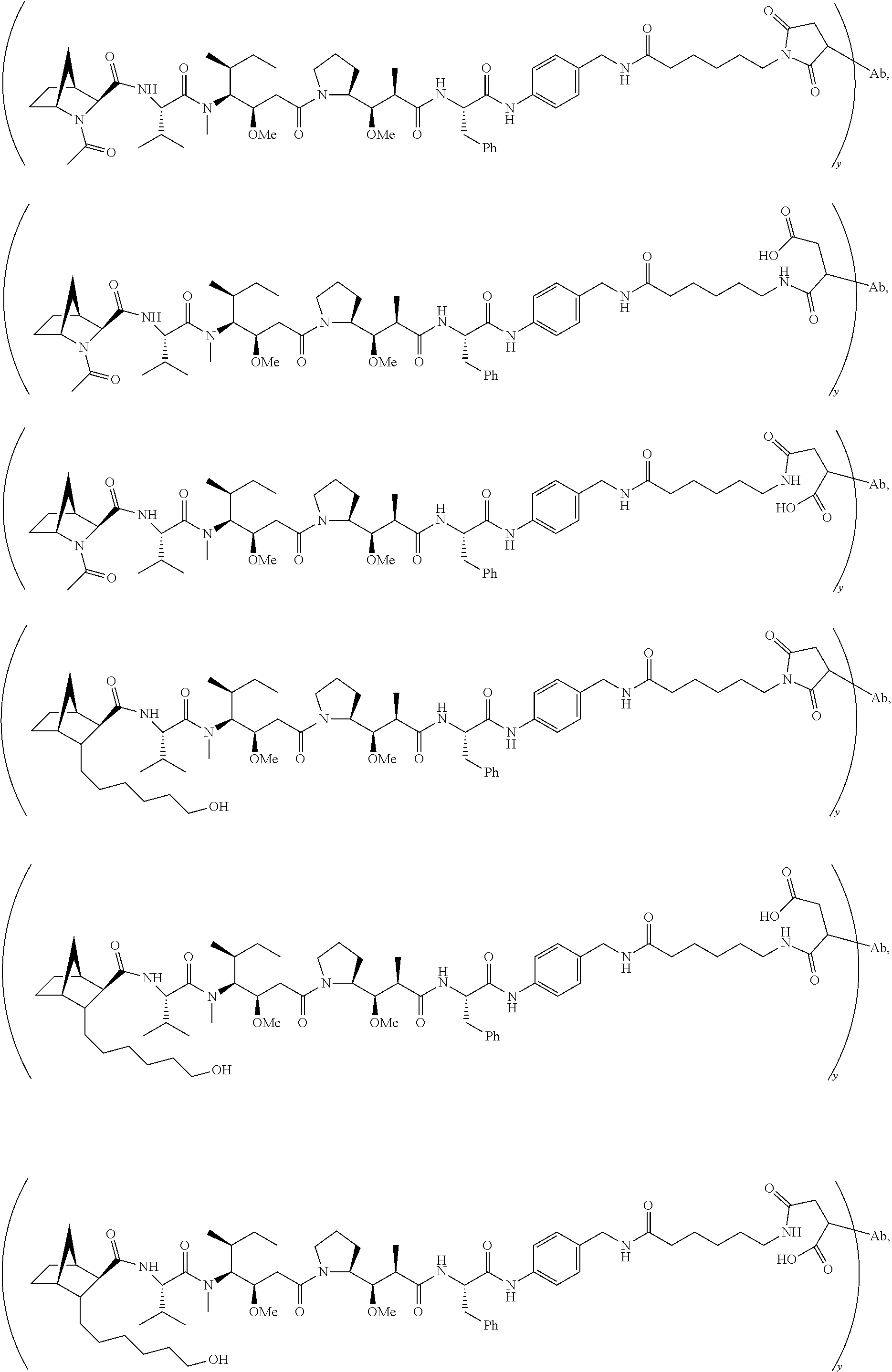

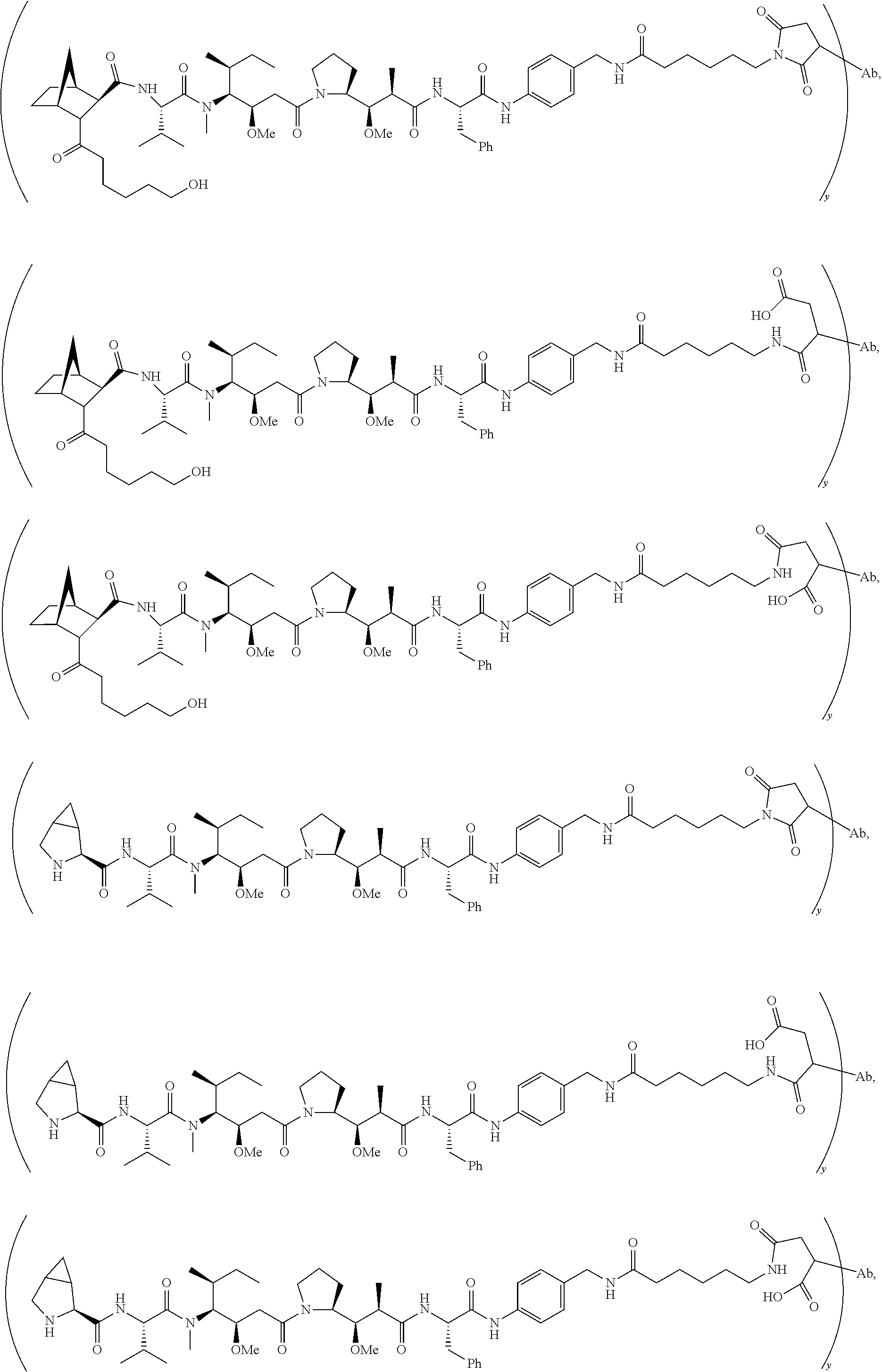

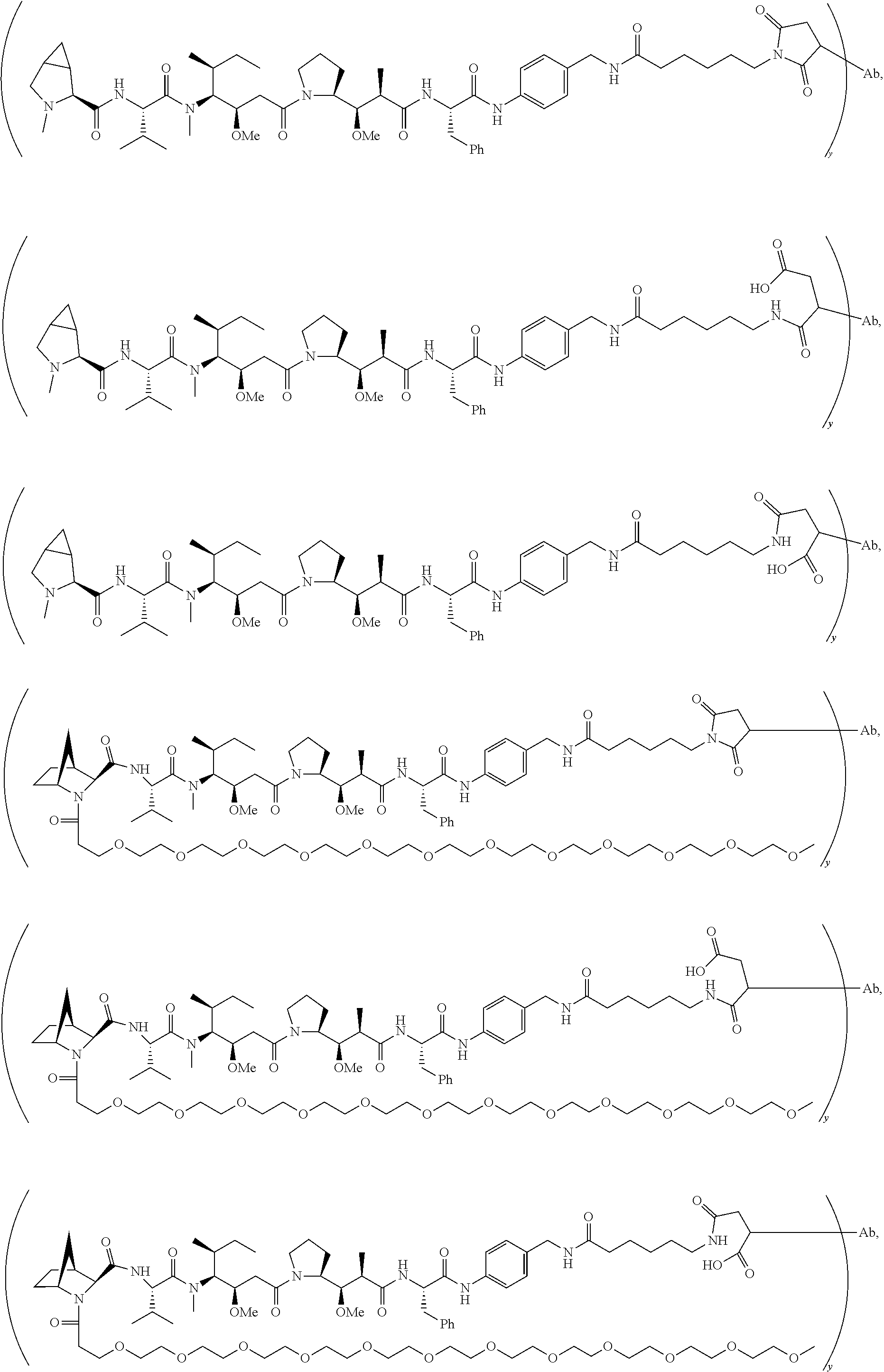

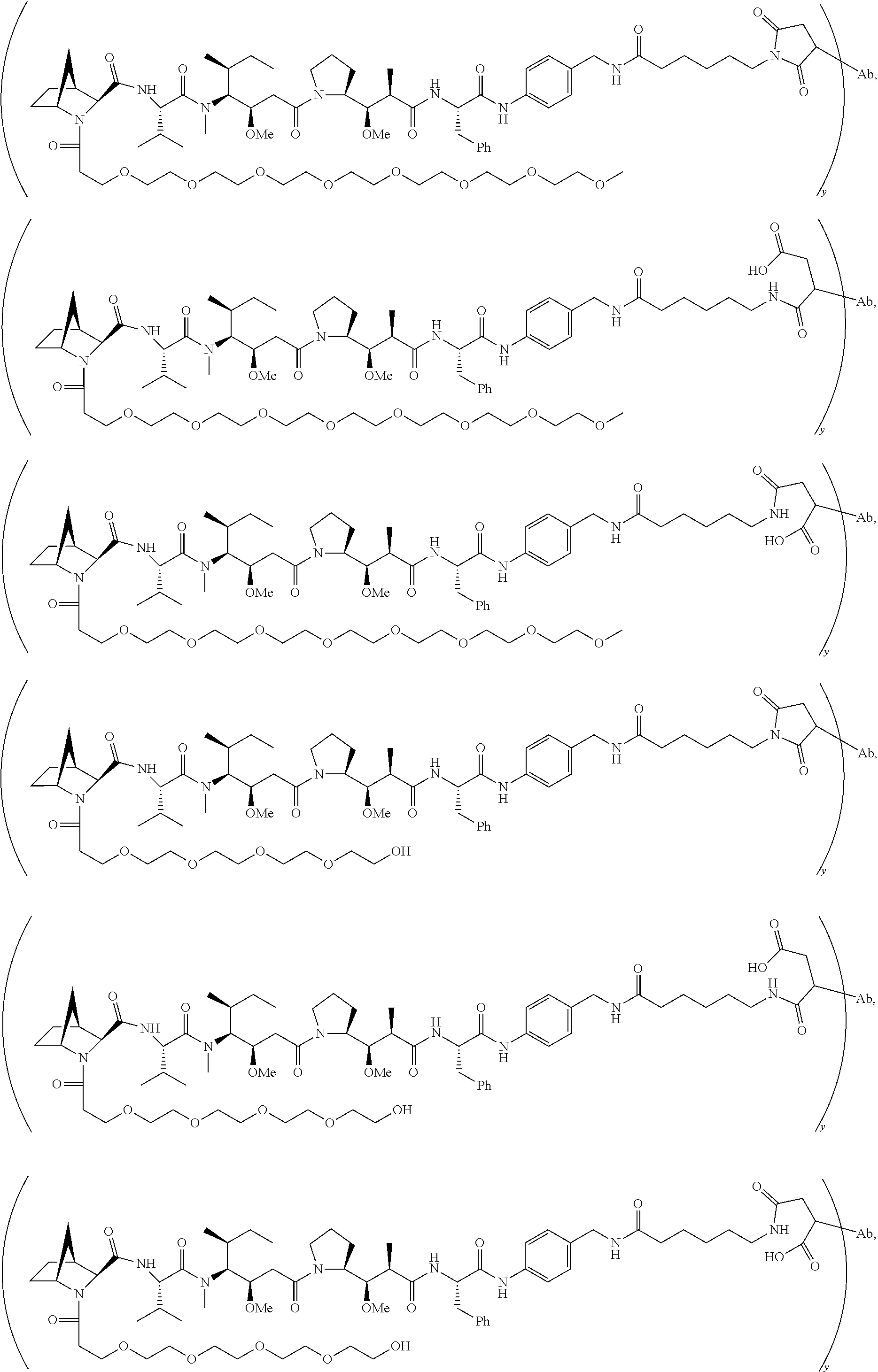

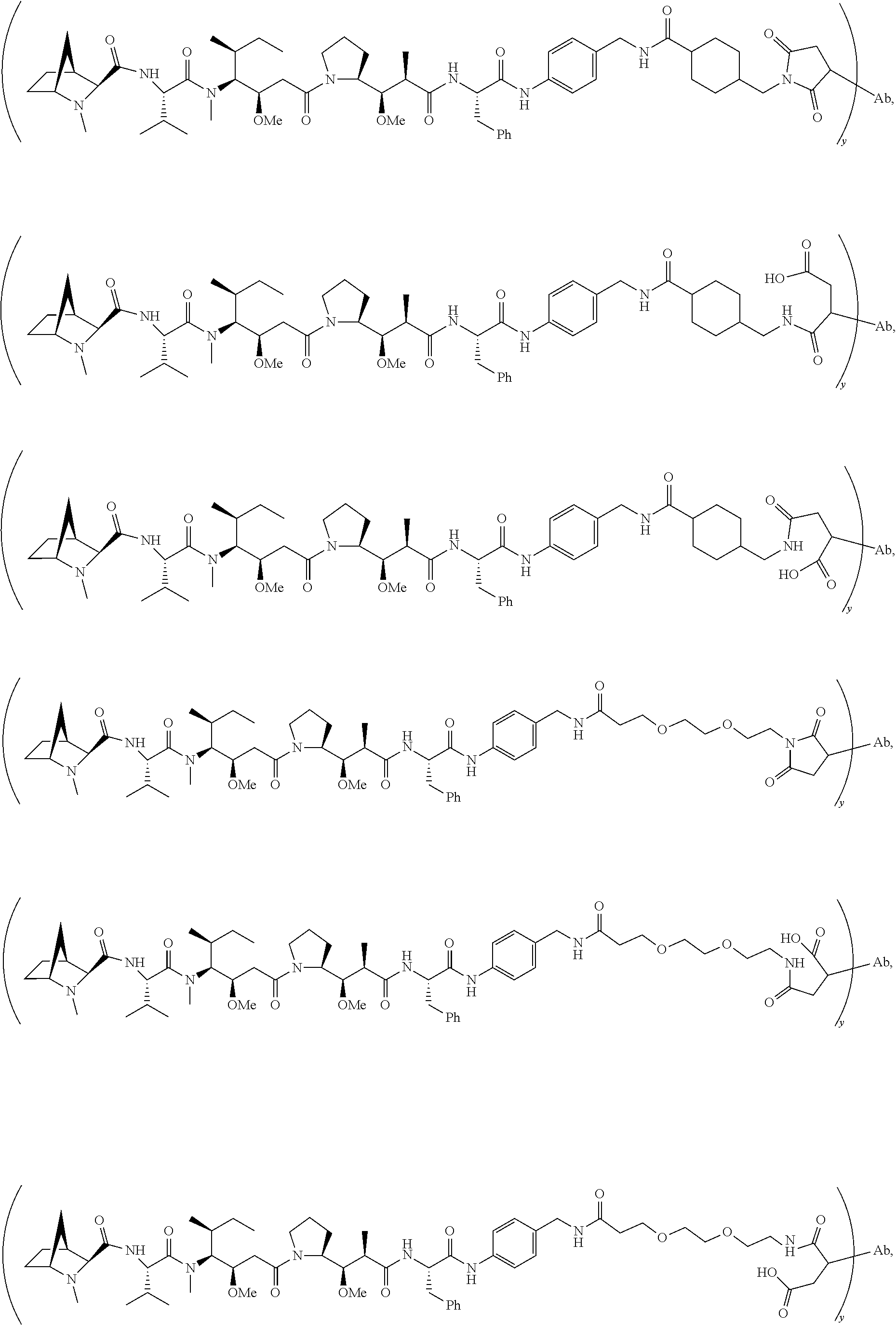

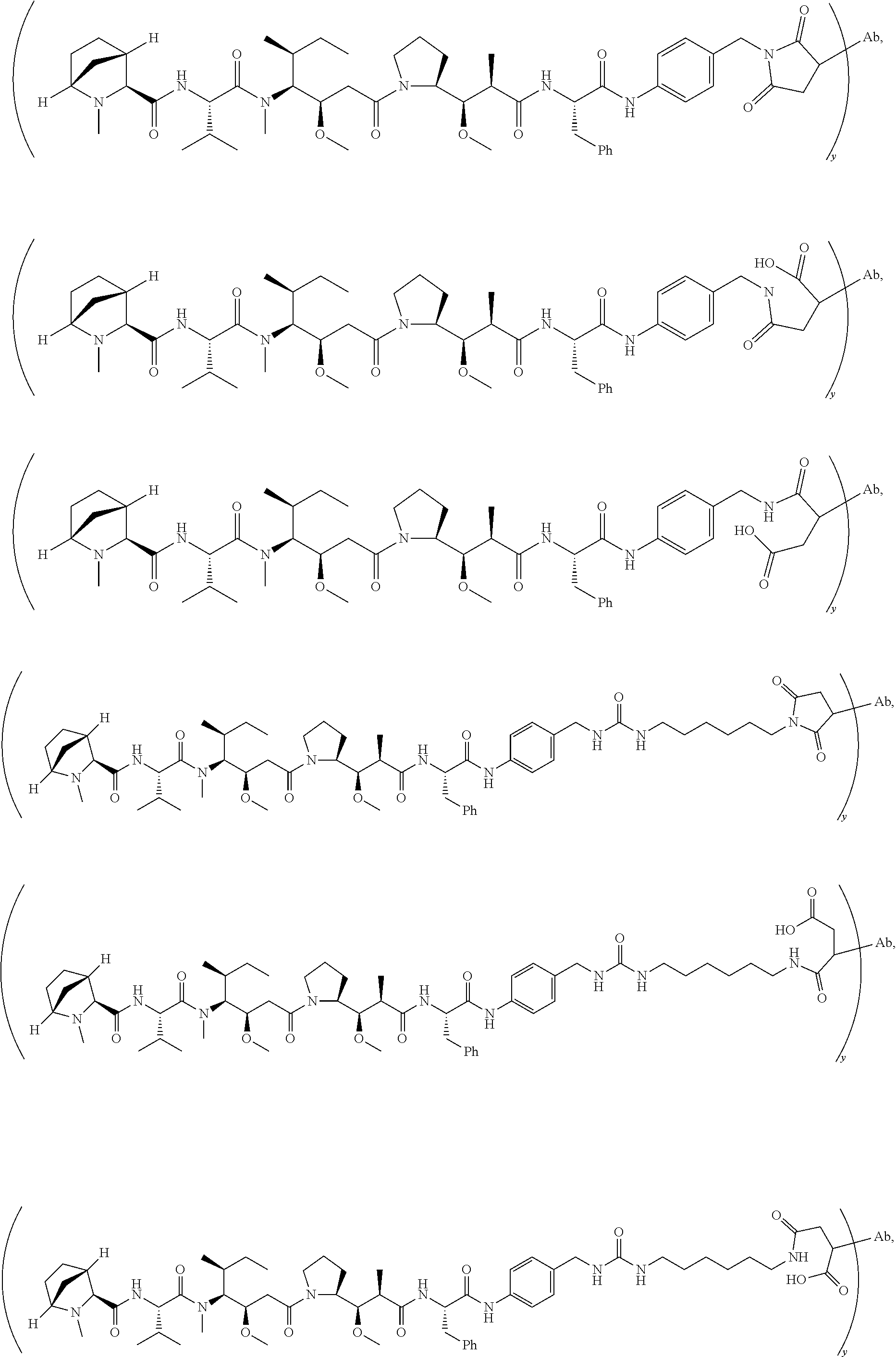

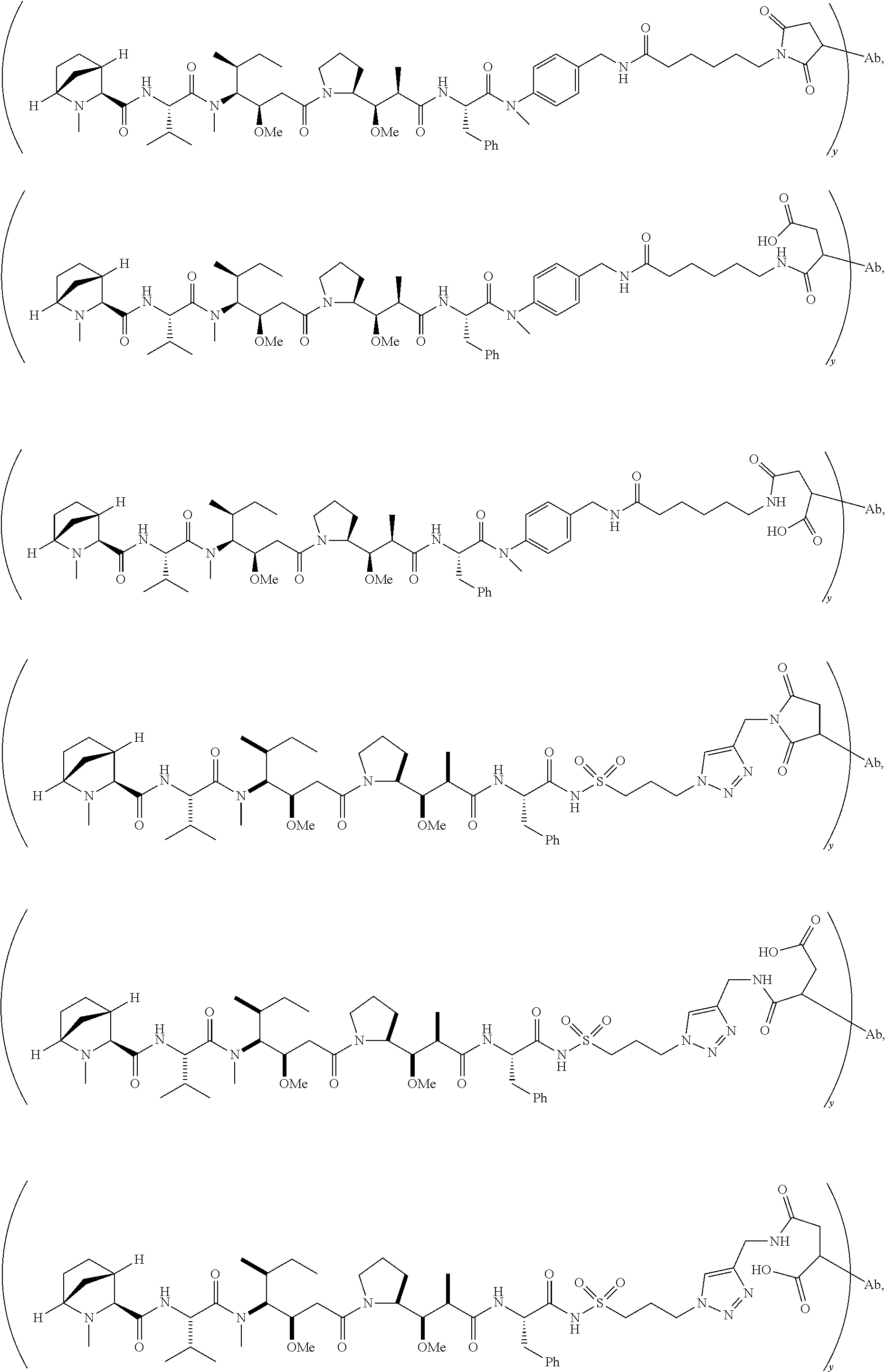

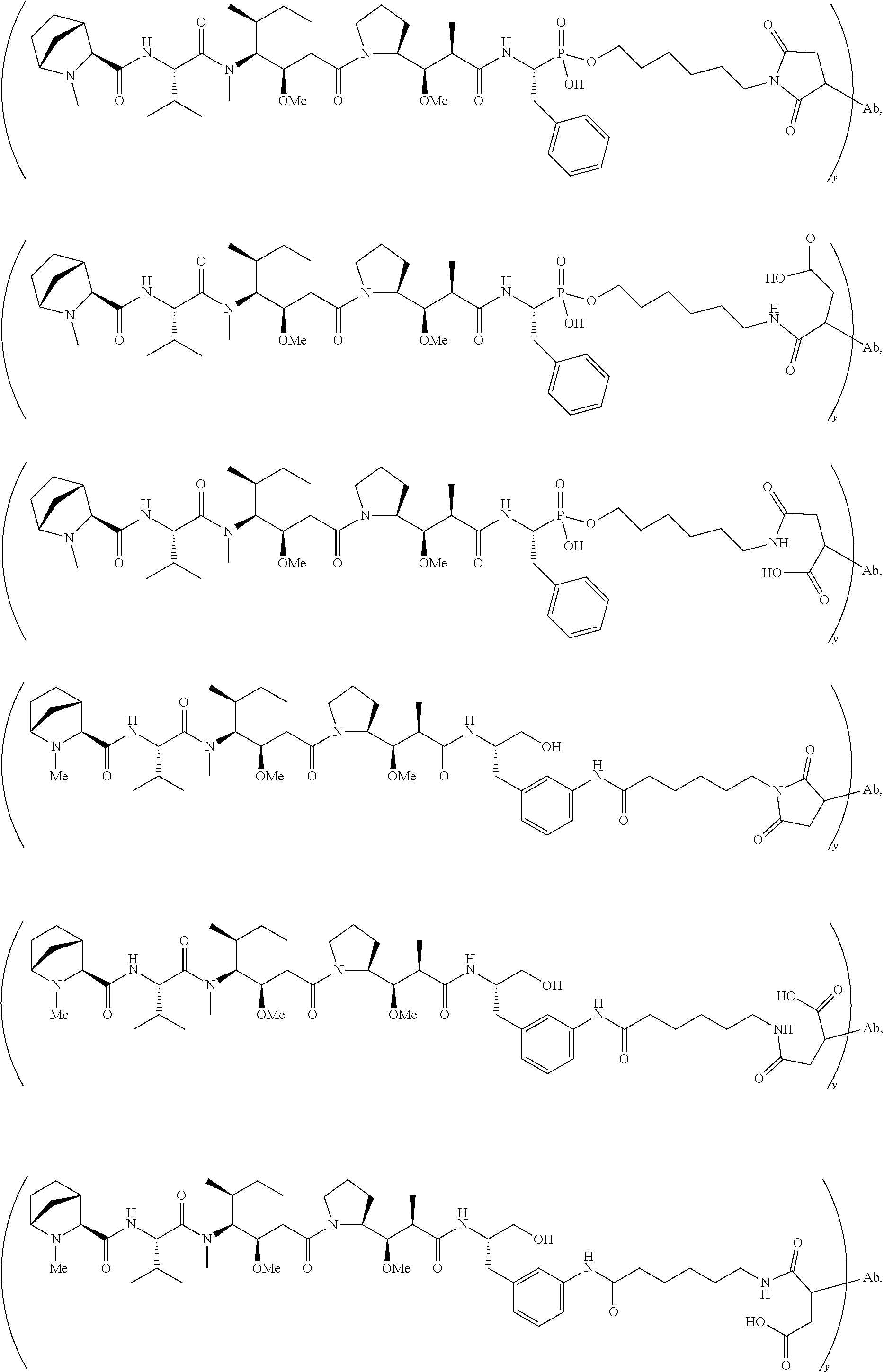

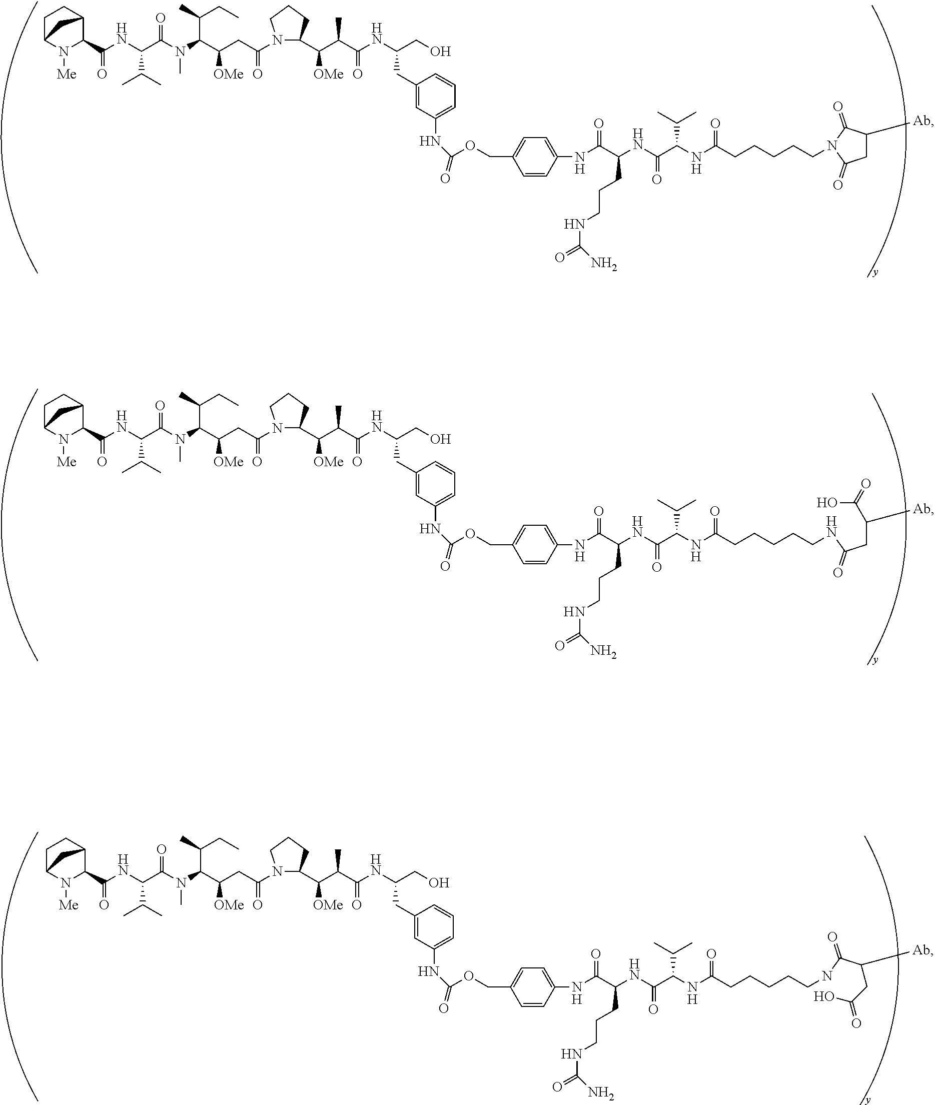

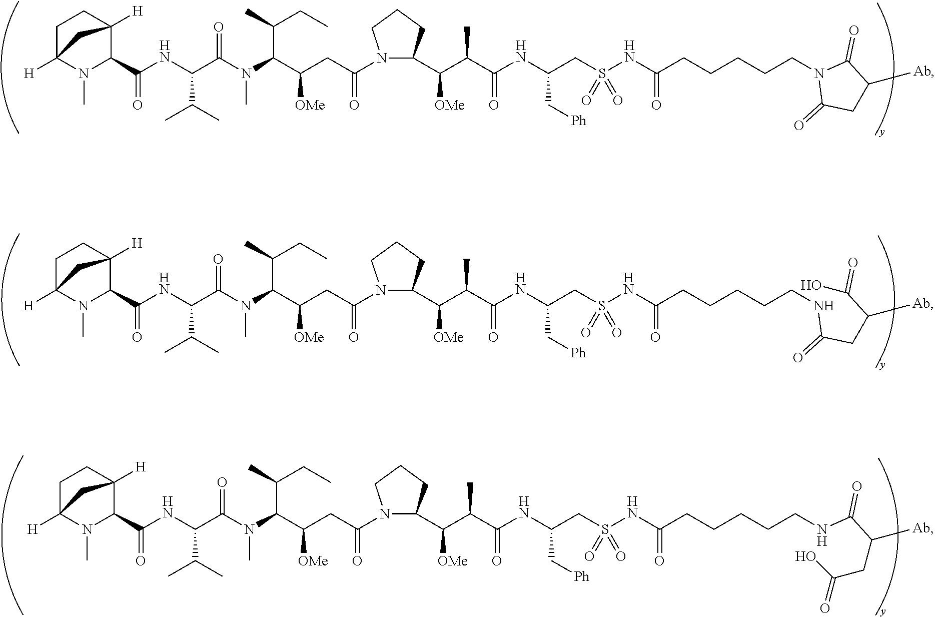

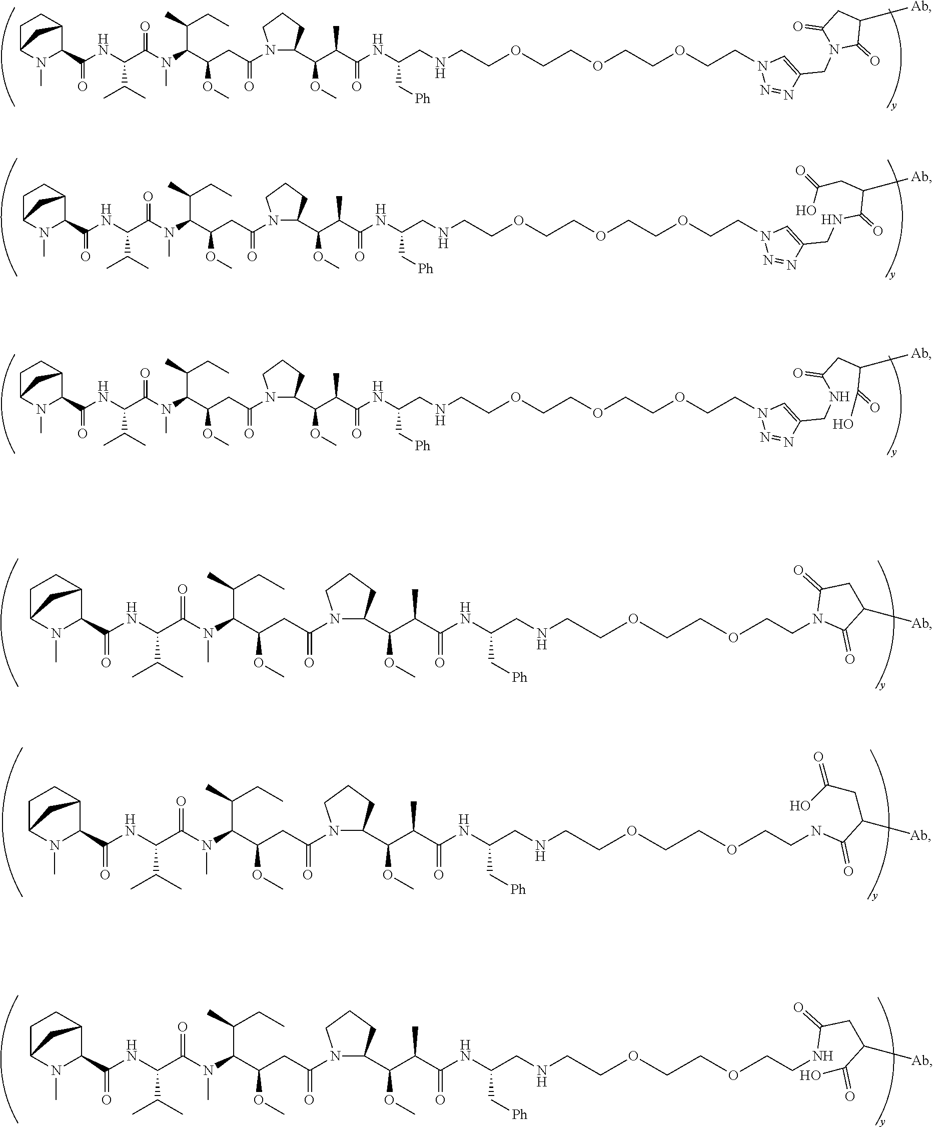

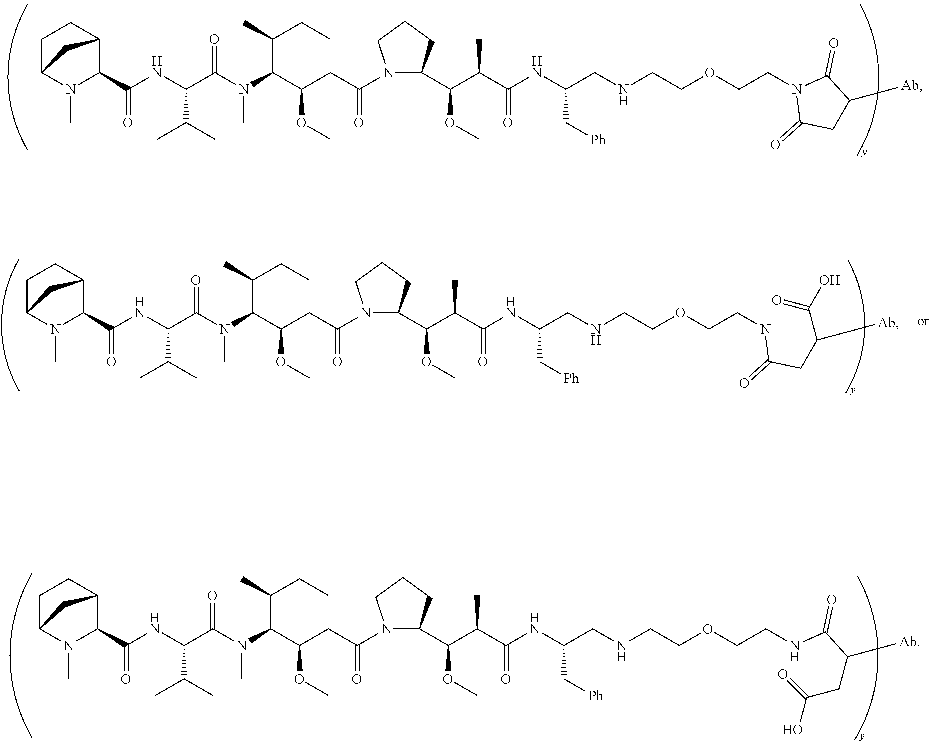

In specific embodiments of the antibody drug conjugates disclosed herein, the conjugates have one of the following structures:

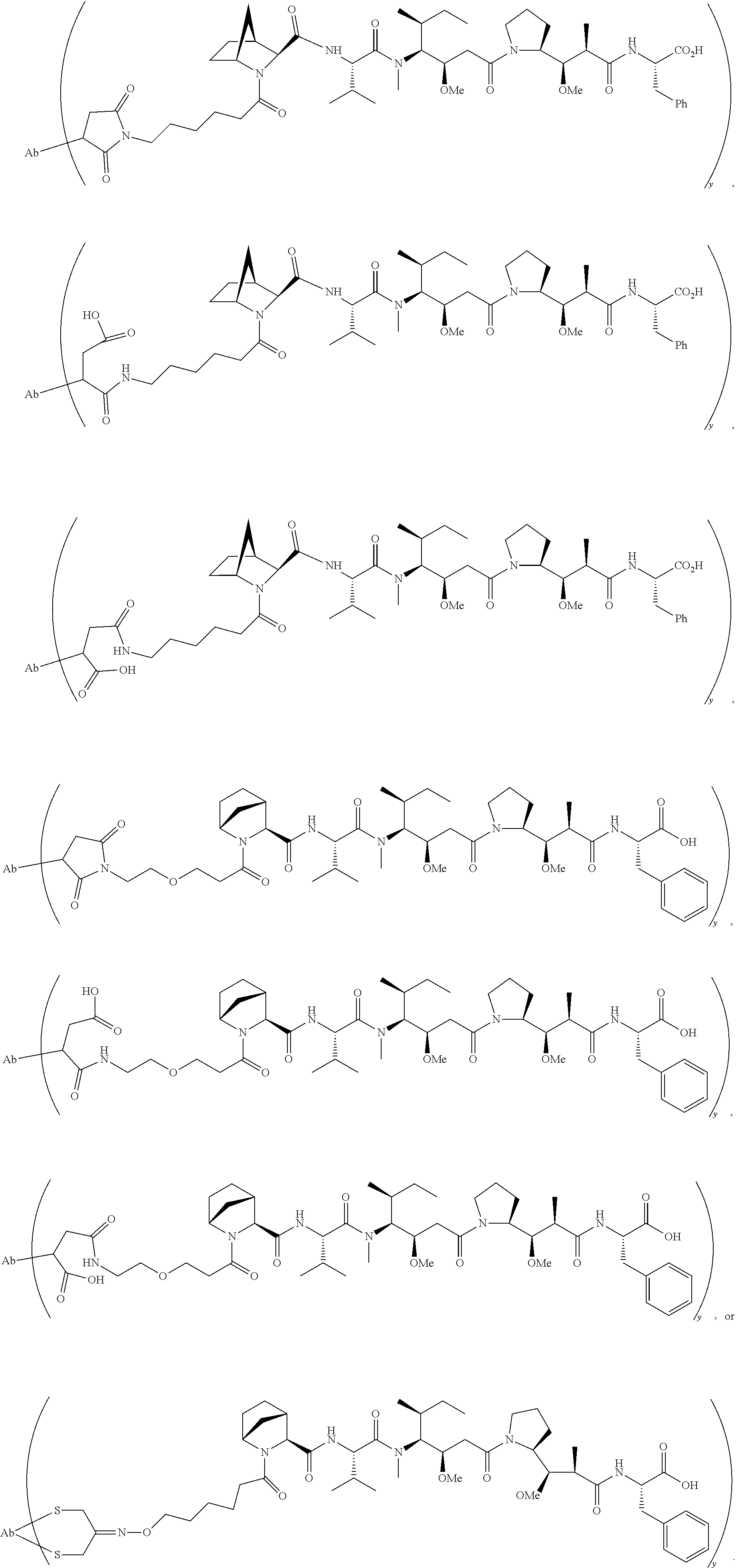

For each of the structures disclosed above:
Ab is an antibody or antigen binding fragment thereof that specifically binds P-cadherin;
y, which indicates the number of D-L groups attached the Ab, is an integer from 1 to 20. In one embodiment, y is an integer from 1 to 10, 2 to 8 or 2 to 5. In a specific embodiment, y is 3 or 4.

In one embodiment, the average molar ratio of drug to the antibody in the conjugate is about 1 to about 10, about 2 to about 8 (e.g., 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, or 8.1), about 2.5 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, or about 4.5 to 5.5 (e.g., about 4.5, about 4.6, about 4.7, about 4.8, about 4.9, about 5.0, about 5.1, about 5.2, about 5.3, about 5.4, or about 5.5).

In an aspect of the invention, the conjugate of the present invention has substantially high purity and has one or more of the following features: (a) greater than about 90% (e.g., greater than or equal to about 91%, 92%, 93%, 94%, 95%, 96%, 97%, 98%, 99%, or 100%), preferably greater than about 95%, of conjugate species are monomeric, (b) unconjugated linker level in the conjugate preparation is less than about 10% (e.g., less than or equal to about 9%, 8%, 7%, 6%, 5%, 4%, 3%, 2%, 1%, or 0%) (relative to total linker), (c) less than 10% of conjugate species are crosslinked (e.g., less than or equal to about 9%, 8%, 7%, 6%, 5%, 4%, 3%, 2%, 1%, or 0%), (d) free drug level in the conjugate preparation is less than about 2% (e.g., less than or equal to about 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (mol/mol relative to total cytotoxic agent) and/or (e) no substantial increase in the level of free drug occurs upon storage (e.g., after about 1 week, about 2 weeks, about 3 weeks, about 1 month, about 2 months, about 3 months, about 4 months, about 5 months, about 6 months, about 1 year, about 2 years, about 3 years, about 4 years, or about 5 years). “Substantial increase” in the level of free drug means that after certain storage time (e.g., about 1 week, about 2 weeks, about 3 weeks, about 1 month, about 2 months, about 3 months, about 4 months, about 5 months, about 6 months, about 1 year, about 2 years, about 3 years, about 4 years, or about 5 years), the increase in the level of free drug is less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.5%, about 2.7%, about 3.0%, about 3.2%, about 3.5%, about 3.7%, or about 4.0%.

As used herein, the term “unconjugated linker” refers to the antibody that is covalently linked with a linker derived from a cross-linking reagent, wherein the antibody is not covalently coupled to the drug through a linker.

### 1. Drug Moiety

The present invention provides immunoconjugates that specifically bind to P-cadherin. The antibody drug conjugates of the invention comprise anti-P-cadherin antibodies, antibody fragments (e.g., antigen binding fragments) or functional equivalents that are conjugated to a drug moiety, e.g., an anti-cancer agent, an autoimmune treatment agent, an anti-inflammatory agent, an antifungal agent, an antibacterial agent, an anti-parasitic agent, an anti-viral agent, or an anesthetic agent. The antibodies, antibody fragments (e.g., antigen binding fragments) or functional equivalents of the invention can be conjugated to several identical or different drug moieties using any methods known in the art.

In certain embodiments, the drug moiety of the immunoconjugates of the present invention is selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an Eg5 inhibitor, proteasome inhibitors, an inhibitor of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In one embodiment, the drug moiety of the immunoconjugates of the present invention comprises a cytoxic peptide. More particularly, the drug moiety is an auristatin analogue, such as those disclosed in PCT/US2014/070800, which is incorporated herein by reference in its entirety. In one embodiment, the drug moiety comprises a cytoxic peptides or auristatin analogue, or any stereoisomer or pharmaceutically acceptable salt thereof having the structure of Formula C:

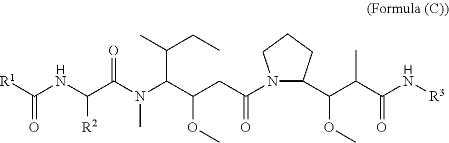

wherein:
R¹ is a C-linked 6 membered heterocycloalkyl containing 1-2 N heteroatoms and a C₁-C₂alkylene bridge or R¹ is a C-linked 5-8 membered fused bicyclic heterocycloalkyl containing 1-2 N heteroatoms, wherein each is unsubstituted, or each is substituted with an R⁷ and 0 to 3 substituents independently selected from R⁵ and R⁶, or each is substituted with 1 to 3 substituents independently selected from R⁵ and R⁶;
R² is —C₁-C₆alkyl;
R³ is

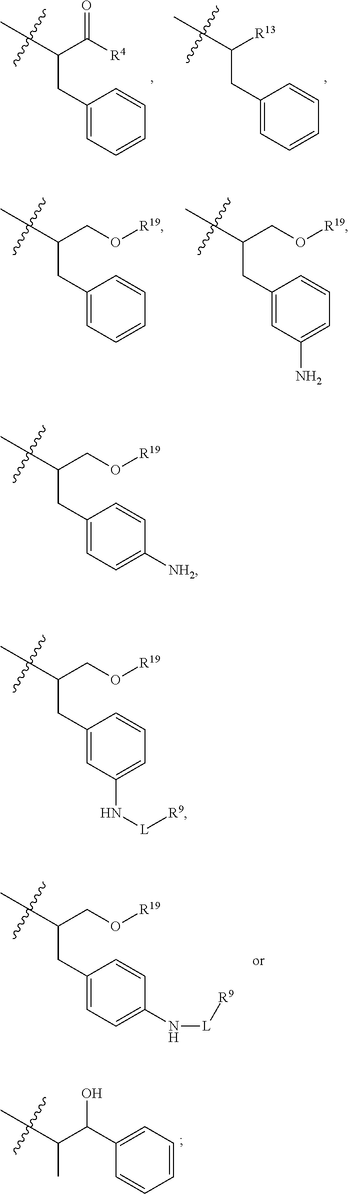

R⁴ is —OH, C₁-C₆alkoxy, —N(R¹⁴)₂, —R¹⁶, —NR¹²(CH₂)ₘN(R¹⁴)₂, —NR¹²(CH₂)ₘR¹⁶, -LR⁹, —NHS(O)₂R₁₁, —NHS(O)₂(CH₂)ₘN₃, —NHS(═O)₂LR⁹,

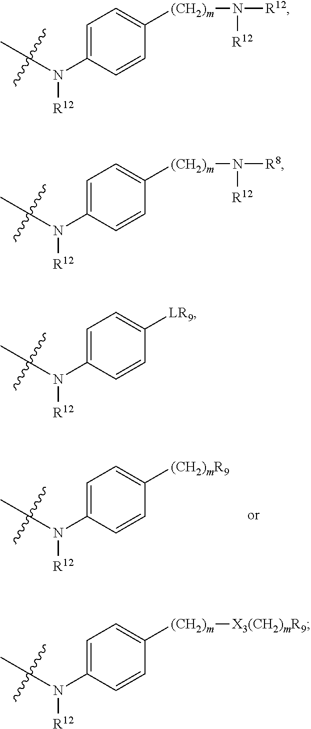

R⁵ is C₁-C₆alkyl, —C(═O)R¹¹, —(CH₂)ₘOH, —C(═O)(CH₂)ₘOH, —C(═O)((CH₂)ₘO)ₙR¹², —((CH₂)ₘO)ₙR¹², or C₁-C₆alkyl which is optionally substituted with —CN, —C(═O)NH₂ or 1 to 5 hydroxyl;
R⁶ is halo, oxo, OH, C₁-C₆alkyl, —N(R¹⁴)₂, —R¹⁶ and —NR¹²C(═O)R¹¹;
R⁷ is LR⁹;
R⁸ is H or LR⁹;
each L is independently selected from -L₁L₂L₃L₄L₅L₆-, -L₆L₅L₄L₃L₂L₁-, -L₁L₂L₃L₄L₅-, -L₅L₄L₃L₂L₁-, -L₁L₂L₃L₄-, -L₄L₃L₂L₁-, -L₁L₂L₃-, -L₃L₂L₁-, -L₁L₂-, -L₂L₁- and -L₁, wherein -L₁, L₂, L₃, L₄, L₅, and L₆ are as defined herein;
R⁹ is

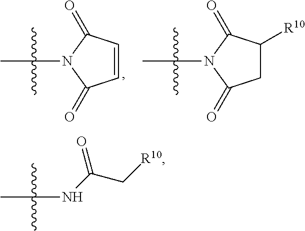

—NR₁₂C(═O)CH═CH₂, —N₃,

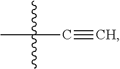 [SMILES: *C#C]

SH, —SSR¹⁵, —S(═O)₂(CH═CH₂), —(CH₂)₂S(═O)₂(CH═CH₂), —NR₁₂S(═O)₂(CH═CH₂), —NR₁₂C(═O)CH₂R¹⁰, —NR₁₂C(═O)CH₂Br, —NR₁₂C(═O)CH₂I, —NHC(═O)CH₂Br, —NHC(═O)CH₂I, —ONH₂, —C(O)NHNH₂,

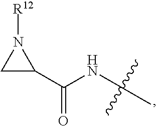

—CO₂H, —NH₂, —NCO, —NCS,

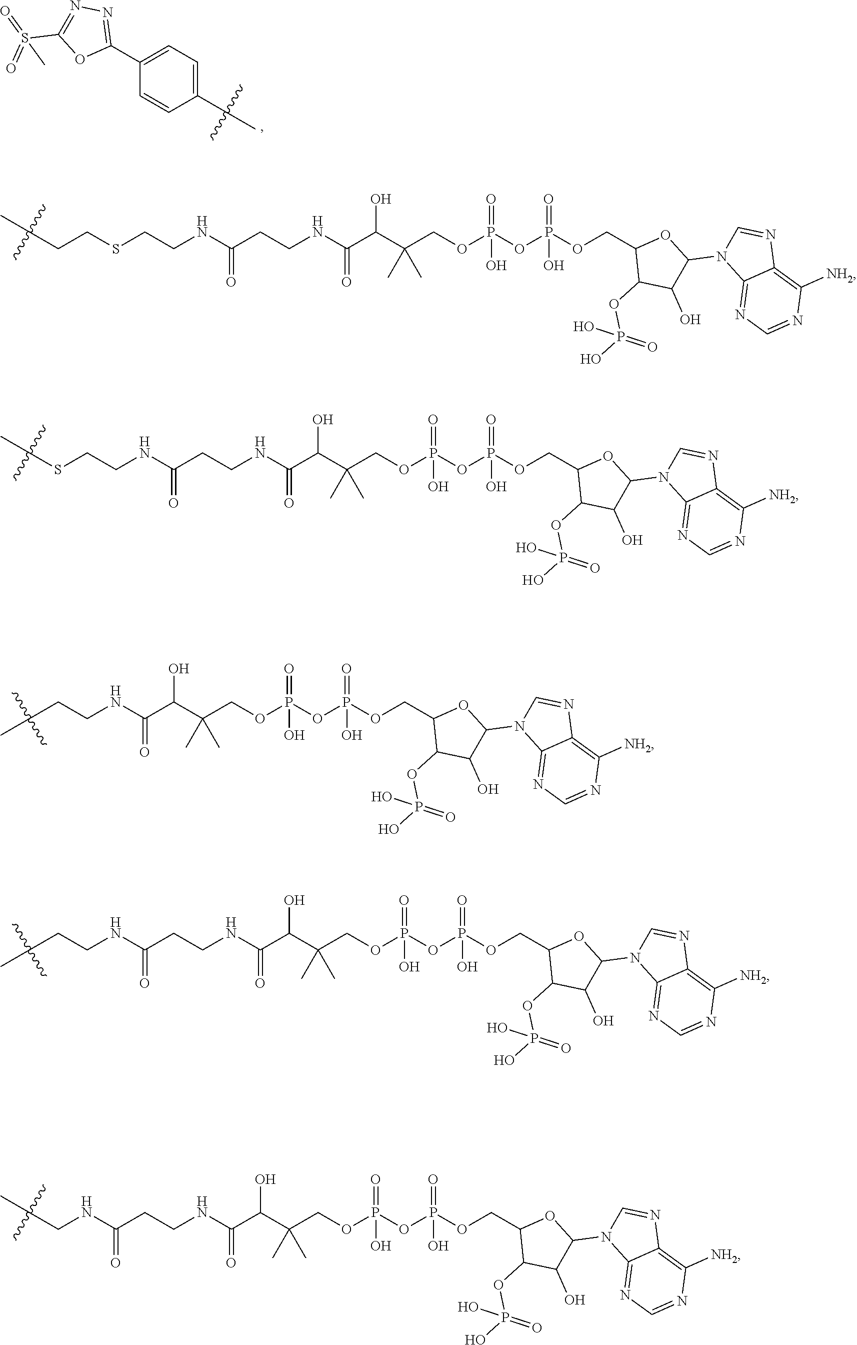 [SMILES: *c1ccc(-c2nnc(S(C)(=O)=O)o2)cc1 ; *CCSCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

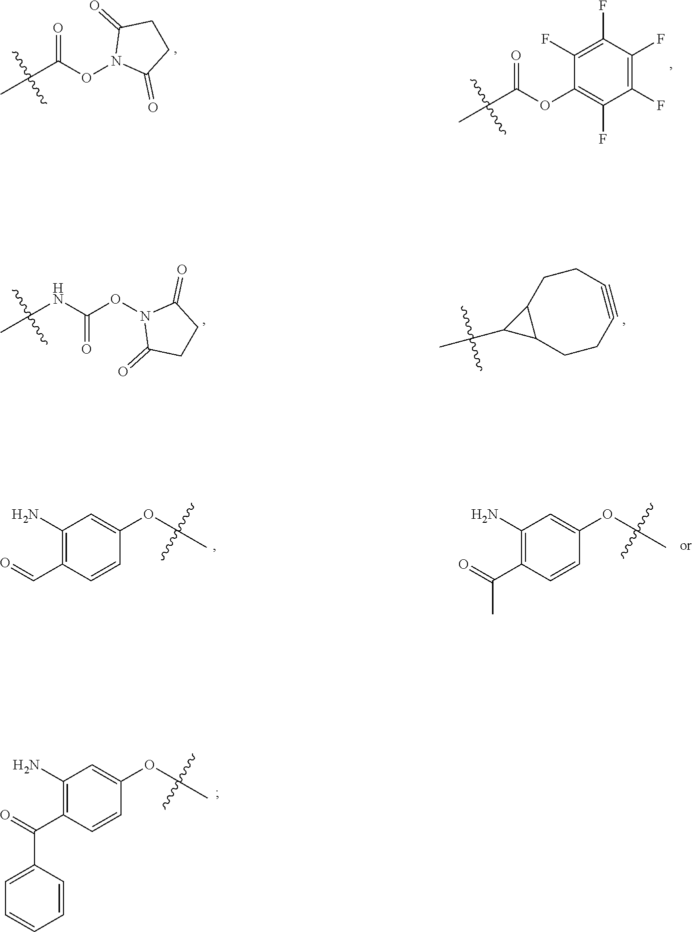

R¹⁰ is

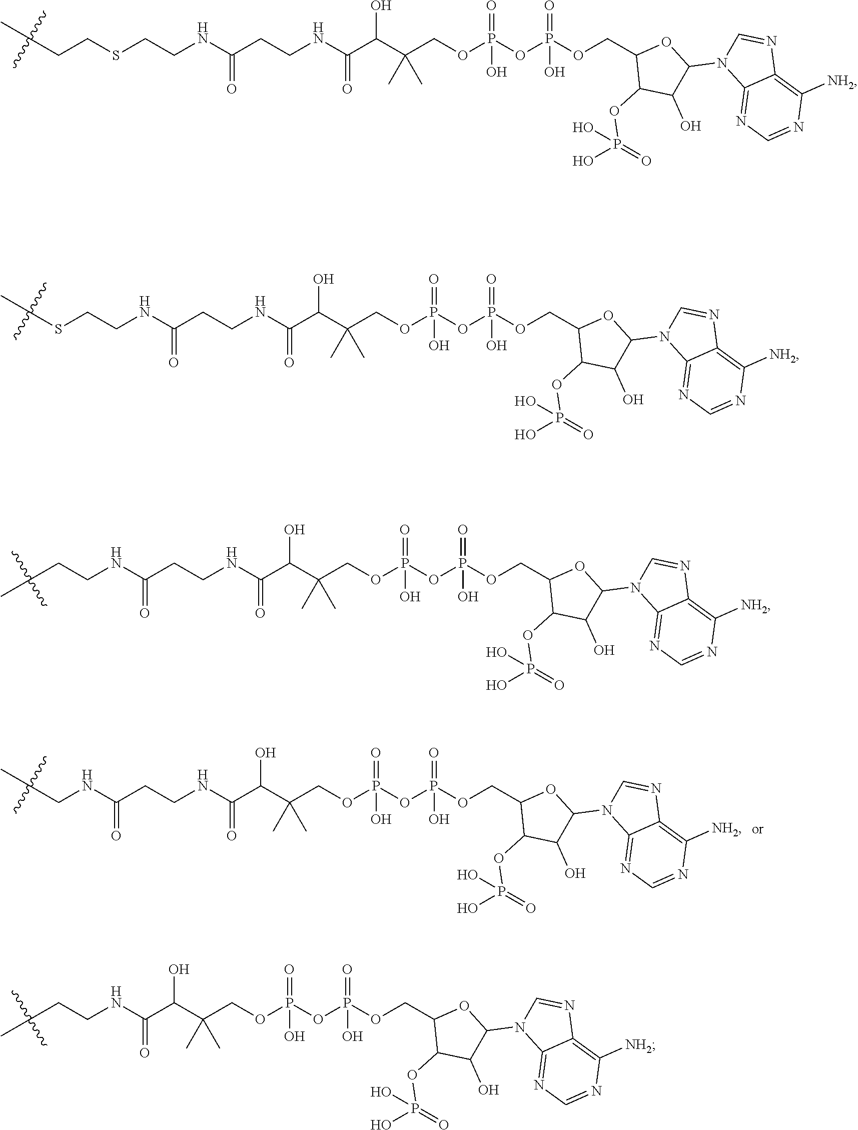 [SMILES: *CCSCCNC(=O)CCNC(=O)C(O)C(C)(C)COP(=O)(O)OP(=O)(O)OCC1OC(n2cnc3c(N)ncnc32)C(O)C1OP(=O)(O)O ; *SCCNC(=O)CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O ; *CCNC(=O)C(O)C(C)(C)COP(=O)(O)OP(=O)(O)OCC1OC(n2cnc3c(N)ncnc32)C(O)C1OP(=O)(O)O]

each R¹¹ is independently selected from C₁-C₆alkyl and C₁-C₆alkyl which is optionally substituted with 1 to 5 hydroxyl;
each R¹² is independently selected from H and C₁-C₆alkyl;
R¹³ is tetrazolyl, imidazolyl substituted with phenyl, oxadiazolyl substituted with phenyl, pyrazolyl, pyrimidinyl,

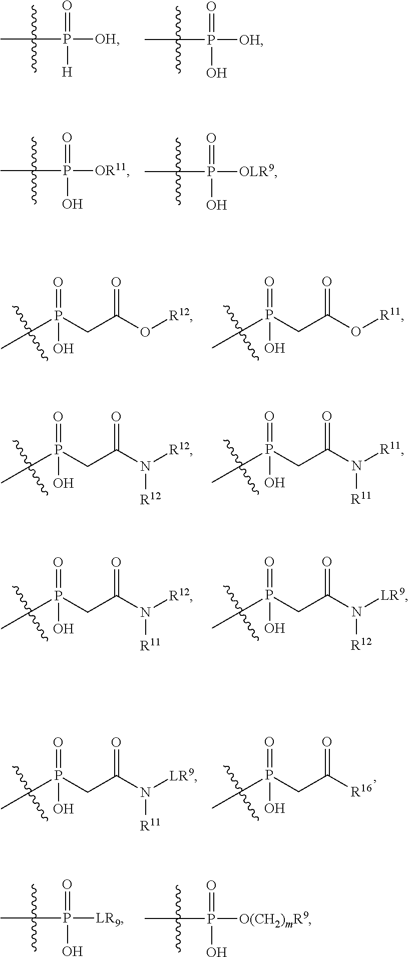

—CH₂S(═O)₂NH₂, —CH₂S(═O)₂NHLR⁹, -LR⁹ or —X₄LR⁹;
each R¹⁴ is independently selected from H and C₁-C₆alkyl;
R¹⁵ is 2-pyridyl or 4-pyridyl;
R¹⁶ is an N-linked 4-8 membered heterocycloalkyl containing 1-2 heteroatoms independently selected from N and O, which is unsubstitituted or substituted with -LR⁹;
each R¹⁹ is H or C₁-C₆alkyl;
X₃ is

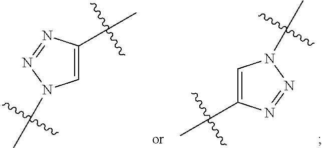 [SMILES: *c1cn(*)nn1 ; *c1cn(*)nn1]

### X₄ is

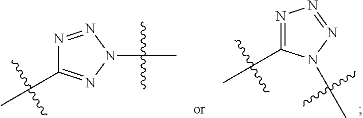 [SMILES: *c1nnn(*)n1 ; *c1nnnn1*] [0336] each m is independently selected from 1, 2, 3, 4, 5, 6, 7, 8, 9 and 10; and
each n is independently selected from 1, 2, 3, 4, 5, 6, 7, 8, 9, 10, 11, 12, 13, 14, 15, 16,17 and 18.

In some embodiments, the drug moiety is selected from any one of the following compounds, which can be conjugated to the P-cadherin antibodies (Ab) disclosed herein via a linker (L) to form the antibody drug conjugates of Formula A or Formula B:

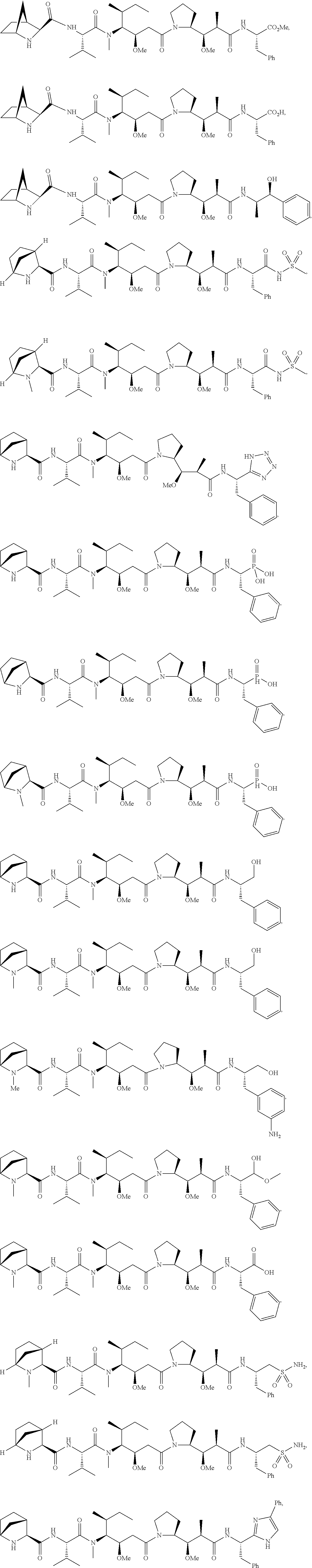

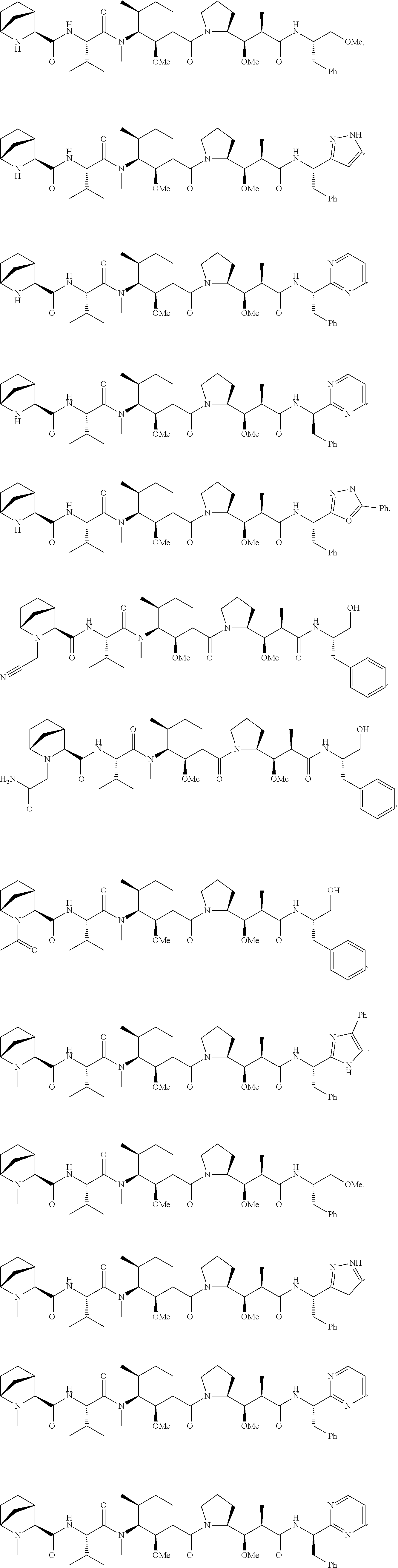

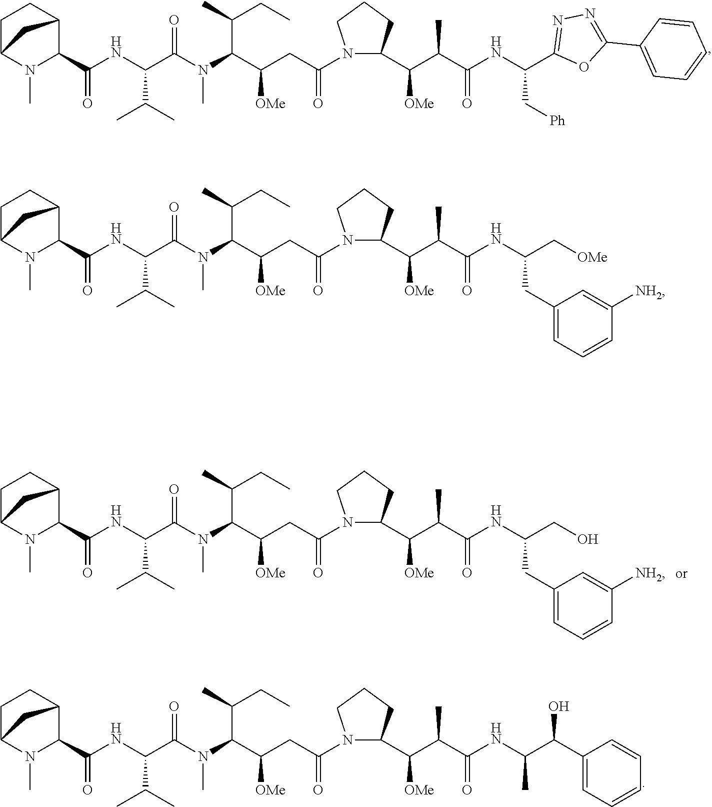

Further the antibodies, antibody fragments (e.g., antigen binding fragments) or functional equivalents of the present invention may be conjugated to a drug moiety that modifies a given biological response. Drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin, a protein such as tumor necrosis factor, α-interferon, β-interferon, nerve growth factor, platelet derived growth factor, tissue plasminogen activator, a cytokine, an apoptotic agent, an anti-angiogenic agent, or, a biological response modifier such as, for example, a lymphokine.

In one embodiment, the antibodies, antibody fragments (e.g., antigen binding fragments) or functional equivalents of the present invention are conjugated to a drug moiety, such as a cytotoxin, a drug (e.g., an immunosuppressant) or a radiotoxin. Examples of cytotoxins include but are not limited to, taxanes (see, e.g., International (PCT) Patent Application Nos. WO 01/38318 and PCT/US03/02675), DNA-alkylating agents (e.g., CC-1065 analogs), anthracyclines, tubulysin analogs, duocarmycin analogs, auristatin E, auristatin F, maytansinoids, and cytotoxic agents comprising a reactive polyethylene glycol moiety (see, e.g., Sasse et al., J. Antibiot. (Tokyo), 53, 879-85 (2000), Suzawa et al., Bioorg. Med. Chem., 8, 2175-84 (2000), Ichimura et al., J. Antibiot. (Tokyo), 44, 1045-53 (1991), Francisco et al., Blood (2003) (electronic publication prior to print publication), U.S. Pat. Nos. 5,475,092, 6,340,701, 6,372,738, and 6,436,931, U.S. Patent Application Publication No. 2001/0036923 A1, Pending U.S. patent application Ser. Nos. 10/024,290 and 10/116,053, and International (PCT) Patent Application No. WO 01/49698), taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, anti-metabolites (e.g., methotrexate, 6-mercaptopurine, 6-thioguanine, cytarabine, 5-fluorouracil decarbazine), ablating agents (e.g., mechlorethamine, thiotepa chlorambucil, meiphalan, carmustine (BSNU) and lomustine (CCNU), cyclophosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics US20090304721).

Other examples of cytotoxins that can be conjugated to the antibodies, antibody fragments (antigen binding fragments) or functional equivalents of the invention include duocarmycins, calicheamicins, maytansines and auristatins, and derivatives thereof.

Various types of cytotoxins, linkers and methods for conjugating therapeutic agents to antibodies are known in the art, see, e.g., Saito et al., (2003) Adv. Drug Deliv. Rev. 55:199-215; Trail et al., (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3:1089-1091; Senter and Springer, (2001) Adv. Drug Deliv. Rev. 53:247-264.

The antibodies, antibody fragments (e.g., antigen binding fragments) or functional equivalents of the present invention can also be conjugated to a radioactive isotope to generate cytotoxic radiopharmaceuticals, referred to as radioimmunoconjugates. Examples of radioactive isotopes that can be conjugated to antibodies for use diagnostically or therapeutically include, but are not limited to, iodine-131, indium-111, yttrium-90, and lutetium-177. Methods for preparing radioimmunoconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the antibodies of the invention. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., (1998) Clin Cancer Res. 4(10):2483-90; Peterson et al., (1999) Bioconjug. Chem. 10(4):553-7; and Zimmerman et al., (1999) Nucl. Med. Biol. 26(8):943-50, each incorporated by reference in their entireties.

The antibodies, antibody fragments (e.g., antigen binding fragments) or functional equivalents of the present invention can also conjugated to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antibody fragment (e.g., antigen binding fragment) described herein (e.g., a Fab fragment, Fd fragment, Fv fragment, F(ab)2 fragment, a VH domain, a VH CDR, a VL domain or a VL CDR) and a heterologous protein, polypeptide, or peptide.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., (1997) Curr. Opinion Biotechnol. 8:724-33; Harayama, (1998) Trends Biotechnol. 16(2):76-82; Hansson et al., (1999) J. Mol. Biol. 287:265-76; and Lorenzo and Blasco, (1998) Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an antigen may be recombined with one or more components, motifs, sections, parts, domains, fragments, etc. of one or more heterologous molecules.

Moreover, the antibodies, antibody fragments (e.g., antigen binding fragments) or functional equivalents of the present invention can be conjugated to marker sequences, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., (1989) Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “FLAG” tag (A. Einhauer et al., J. Biochem. Biophys. Methods 49: 455-465, 2001). According to the present invention, antibodies or antigen binding fragments can also be conjugated to tumor-penetrating peptides in order to enhance their efficacy.

In other embodiments, the antibodies, antibody fragments (e.g., antigen binding fragments) or functional equivalents of the present invention are conjugated to a diagnostic or detectable agent. Such immunoconjugates can be useful for monitoring or prognosing the onset, development, progression and/or severity of a disease or disorder as part of a clinical testing procedure, such as determining the efficacy of a particular therapy. Such diagnosis and detection can be accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ⁶⁴Cu, ¹¹³Sn, and ¹¹⁷Sn; and positron emitting metals using various positron emission tomographies, and non-radioactive paramagnetic metal ions.

The antibodies, antibody fragments (e.g., antigen binding fragments) or functional equivalents of the invention may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 2. Linker

As used herein, a “linker” is any chemical moiety that is capable of linking an antibody, antibody fragment (e.g., antigen binding fragments) or functional equivalent to another moiety, such as a drug moeity. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid based linker.

Non-cleavable linkers are any chemical moiety capable of linking a drug, such as a maytansinoid, to an antibody in a stable, covalent manner and does not fall off under the categorties listed above for cleaveable linkers. Thus, non-cleavable linkers are substantially resistant to acid-induced cleavage, photo-induced cleavage, peptidase-induced cleavage, esterase-induced cleavage and disulfide bond cleavage. Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which the drug, such as maytansionoid or the antibody does not lose its activity.

Acid-labile linkers are linkers cleavable at acidic pH. For example, certain intracellular compartments, such as endosomes and lysosomes, have an acidic pH (pH 4-5), and provide conditions suitable to cleave acid-labile linkers.

Photo-labile linkers are linkers that are useful at the body surface and in many body cavities that are accessible to light. Furthermore, infrared light can penetrate tissue.

Some linkers can be cleaved by peptidases, i.e. peptidase cleavable linkers. Only certain peptides are readily cleaved inside or outside cells, see e.g. Trout et al., 79 Proc. Natl. Acad. Sci. USA, 626-629 (1982) and Umemoto et al. 43 Int. J. Cancer, 677-684 (1989). Furthermore, peptides are composed of α-amino acids and peptidic bonds, which chemically are amide bonds between the carboxylate of one amino acid and the amino group of a second amino acid. Other amide bonds, such as the bond between a carboxylate and the α-amino group of lysine, are understood not to be peptidic bonds and are considered non-cleavable.

Some linkers can be cleaved by esterases, i.e. esterase cleavable linkers. Again, only certain esters can be cleaved by esterases present inside or outside of cells. Esters are formed by the condensation of a carboxylic acid and an alcohol. Simple esters are esters produced with simple alcohols, such as aliphatic alcohols, and small cyclic and small aromatic alcohols.

Procharged linkers are derived from charged cross-linking reagents that retain their charge after incorporation into an antibody drug conjugate. Examples of procharged linkers can be found in US 2009/0274713.

The cytotoxic peptides provided herein for use as ADC payloads can be attached to a linker, L, or directly to an antigen binding moiety. Suitable linkers for use in such ADCs are well known in the art, and can be used in the conjugates of the invention. The linker, L, can be attached to the antigen binding moiety at any suitable available position on the antigen binding moiety: typically, L is attached to an available amino nitrogen atom (i.e., a primary or secondary amine, rather than an amide) or a hydroxylic oxygen atom, or to an available sulfhydryl, such as on a cysteine. The attachment of the linker, L, to the cytotoxic peptides provided herein can be at the N-terminus of the cytotoxic peptide or at the C-terminus of the cytotoxic peptide. A wide variety of linkers for use in ADCs are known (see, e.g., Lash, Antibody-Drug Conjugates: the Next Generation of Moving Parts, Start-Up, December 2011, 1-6), and can be used in conjugates within the scope of the invention.

The linker, L, of the compounds disclosed herein is a linking moiety comprising one or more linker components L₁, L₂, L₃, L₄, L₅, L₆, etc. In certain embodiments a linker component can represent a bond connecting the groups flanking it together. In certain embodiments, L is -*L₁L₂L₃L₄L₅L₆-, where the * denotes the site of attachment to the cytotoxic peptide of the invention. In certain embodiments a linker component can represent a bond connecting the groups flanking it together. In certain embodiments, L is -*L₁L₂L₃L₄L₅-, where the * denotes the site of attachment to the cytotoxic peptide of the invention. In certain embodiments a linker component can represent a bond connecting the groups flanking it together. In certain embodiments, L is -*L₁L₂L₃L₄-, where the * denotes the site of attachment to the cytotoxic peptide of the invention. In certain embodiments a linker component can represent a bond connecting the groups flanking it together. In certain embodiments, L is -*L₁L₂L₃-, where the * denotes the site of attachment to the cytotoxic peptide of the invention. In a preferred embodiment L is -*L₁L₂-, where the * denotes the site of attachment to the cytotoxic peptide of the invention. In certain embodiment L is -L₁-. Some preferred linkers and linker components are depicted herein.

The linker, L, may be divalent, meaning it can used to link only one payload per linker to an antigen binding moiety, or it can be trivalent an is able to link two payloads per linker to an antigen binding moiety, or it can be polyvalent. Trivalent, tetravalent, and polyvalent linkers can be used to increase the loading of a payload (drug) on an antigen binding moiety (e.g. an antibody), thereby increasing the drug to antibody ratio (DAR) without requiring additional sites on the antibody for attaching multiple linkers. Examples of such linkers given in Bioconjugate Chem., 1999 March-April; 10(2):279-88; U.S. Pat. No. 6,638,499; Clin Cancer Res Oct. 15, 2004 10; 7063; and WO2012/113847A1.

A linker, L, of the compounds disclosed herein can be cleavable or non-cleavable. Cleavable linkers, such as those containing a hydrazone, a disulfide, the dipeptide Val-Cit, and ones containing a glucuronidase-cleavable p-aminobenzyloxycarbonyl moiety, are well known in the art, and can be used. See, e.g., Ducry, et al., Bioconjugate Chem., vol. 21, 5-13 (2010). For the immunoconjugates of comprising a cleavable linker, the linker is substantially stable in vivo until the immunoconjugate binds to or enters a cell, at which point either intracellular enzymes or intracellular chemical conditions (pH, reduction capacity) cleave the linker to free the cytotoxic peptide.

Alternatively, non-cleavable linkers can be used with the compounds disclosed herein. Non-cleavable linkers lack structural components designed to degrade in cells, and thus their structures can vary substantially. See, e.g., Ducry, et al., Bioconjugate Chem., vol. 21, 5-13 (2010). These immunoconjugates are believed to enter a targeted cell and undergo proteolytic degradation of the antibody rather than linker decomposition; thus at least a portion, or all, of the linker and even some of the antibody or antibody fragment may remain attached to the payload.

The linker, L, of the compounds disclosed herein typically commonly contain two or more linker components, which may be selected for convenience in assembly of the conjugate, or they may be selected to impact properties of the conjugate. Suitable linker components for forming linker, L, are known in the art, as are methods for constructing the linker L. Linker components can include the groups commonly used to attach a group to an amino acid, spacers such as alkylene groups and ethylene oxide oligomers, amino acids and short peptides up to about 4 amino acids in length; a bond; and carbonyl, carbamate, carbonate, urea, ester and amide linkages, and the like. Linker components can comprise thiol-maleimide groups, thioethers, amides, and esters; groups that are easily cleaved in vivo under conditions found in, on or around targeted cells, such as disulfides, hydrazones, dipeptides like Val-Cit, substituted benzyloxycarbonyl groups, and the like; spacers to orient the payload in a suitable position relative to the antigen binding moiety, such as phenyl, heteroaryl, cycloalkyl or heterocyclyl rings, and alkylene chains; and/or pharmacokinetic property-enhancing groups, such as alkylene substituted with one or more polar groups (carboxy, sulfonate, hydroxyl, amine, amino acid, saccharide), and alkylene chains containing one or more —NH— or —O— in place of methylene group(s), such as glycol ethers (—CH₂CH₂O—)ₚ where p is 1-10, which may enhance solubility or reduce intermolecular aggregation, for example.

In addition, linker components can comprise chemical moieties that are readily formed by reaction between two reactive groups. Non-limiting examples of such chemical moieties are given in Table 1.

TABLE 1

| Reactive Group 1 | Reactive Group 2 | Chemical Moiety |
|---|---|---|
| a thiol | a thiol | —S—S— |
| a thiol | a maleimide | 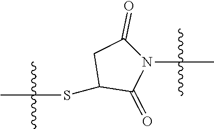 [SMILES: *SC1CC(=O)N(*)C1=O] |
| a thiol | a haloacetamide | 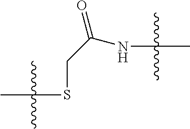 [SMILES: *NC(=O)CS*] |
| an azide | an alkyne | 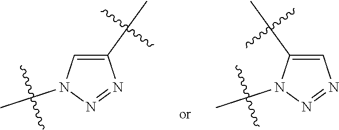 [SMILES: *c1cn(*)nn1 ; *c1cnnn1*] |
| an azide | a triaryl phosphine | 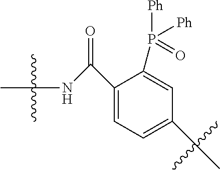 [SMILES: *NC(=O)c1ccc(*)cc1P(=O)(c1ccccc1)c1ccccc1] |
| an azide | a cyclooctene | 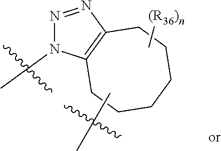 |
| | | 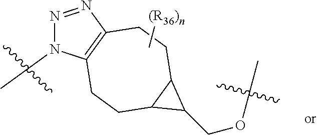 |
| | | 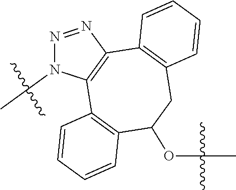 [SMILES: *OC1Cc2ccccc2-c2nnn(*)c2-c2ccccc21] |
| an azide | an oxanobornadiene | 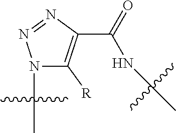 |
| a triaryl phosphine | an azide | 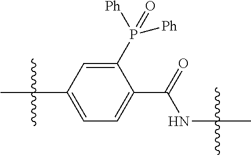 [SMILES: *NC(=O)c1ccc(*)cc1P(=O)(c1ccccc1)c1ccccc1] |
| an oxanobornadiene | an azide | 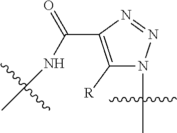 |
| an alkyne | an azide | 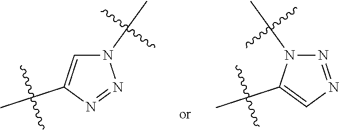 [SMILES: *c1cn(*)nn1 ; *c1cnnn1*] |
| a cyclooctene | azide | 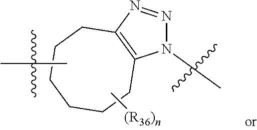 |
| | | 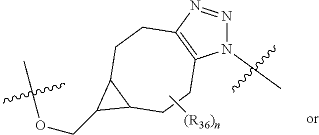 |
| | | 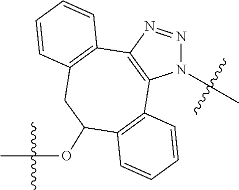 [SMILES: *OC1Cc2ccccc2-c2nnn(*)c2-c2ccccc21] |
| a cyclooctene | a diaryl tetrazine | 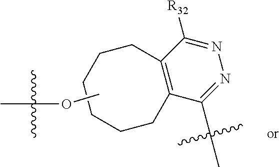 |
| | | 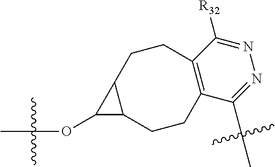 |
| a diaryl tetrazine | a cyclooctene | 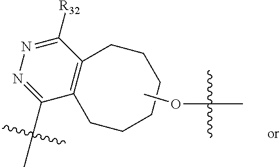 |
| | | 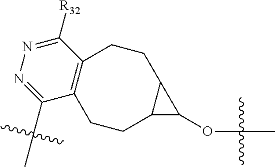 |
| a monoaryl tetrazine | a norbornene | 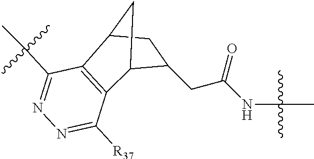 |
| a norbornene | a monoaryl tetrazine | 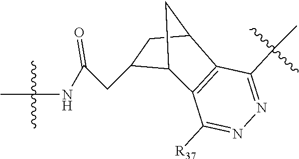 |
| an aldehyde | a hydroxylamine | 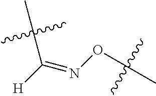 [SMILES: */C=N\O*] |
| an aldehyde | a hydrazine | 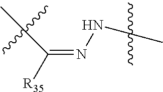 |
| an aldehyde | NH₂—NH—C(═O)— | 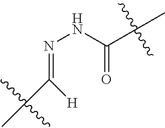 [SMILES: */C=N/NC(*)=O] |
| a ketone | a hydroxylamine | 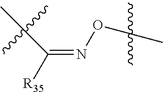 |
| a ketone | a hydrazine | 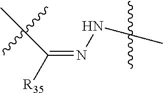 |
| a ketone | NH₂—NH—C(═O)— | 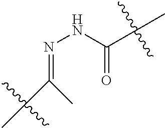 [SMILES: *C(=O)N/N=C(/*)C] |
| a hydroxylamine | an aldehyde | 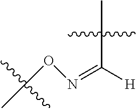 [SMILES: */C=N\O*] |
| a hydroxylamine | a ketone | 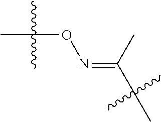 [SMILES: *O/N=C(/*)C] |
| a hydrazine | an aldehyde | 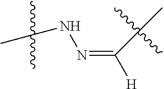 [SMILES: */C=N\N*] |
| a hydrazine | a ketone | 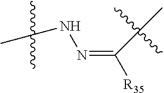 |
| NH₂—NH—C(═O)— | an aldehyde | 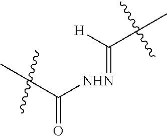 |
| NH₂—NH—C(═O)— | a ketone | 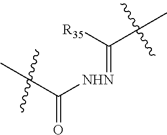 |
| a haloacetamide | a thiol | 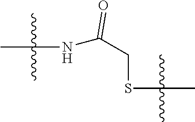 [SMILES: *NC(=O)CS*] |
| a maleimide | a thiol | 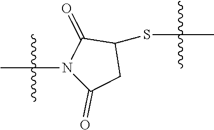 [SMILES: *SC1CC(=O)N(*)C1=O] |
| a vinyl sulfone | a thiol | 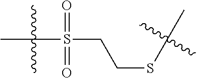 [SMILES: *SCCS(*)(=O)=O] |
| a thiol | a vinyl sulfone | 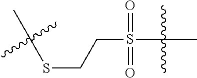 [SMILES: *SCCS(*)(=O)=O] |
| an aziridine | a thiol | 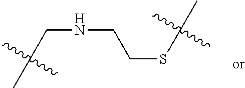 [SMILES: *CNCCS*] |
| | | 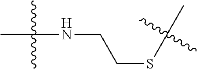 [SMILES: *NCCS*] |
| a thiol | an aziridine | 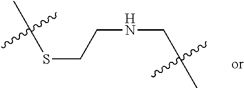 [SMILES: *CNCCS*] |
| | | 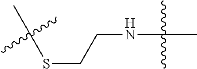 [SMILES: *NCCS*] |
| 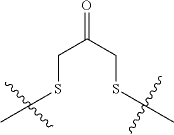 [SMILES: *SCC(=O)CS*] | hydroxylamine | 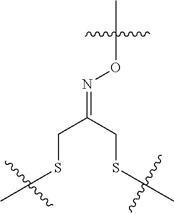 [SMILES: *ON=C(CS*)CS*] |
| 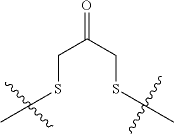 [SMILES: *SCC(=O)CS*] | hydroxylamine | 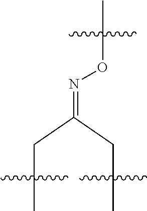 [SMILES: *CC(C*)=NO*] |
| 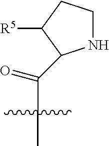 | 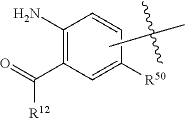 | 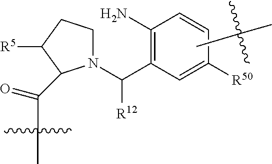 |
| 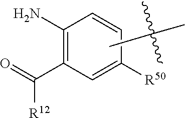 | 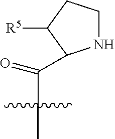 | 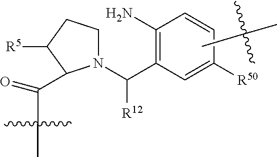 |

where: R³² in Table 1 is H, C₁₋₄ alkyl, phenyl, pyrimidine or pyridine; R³⁵ in Table 1 is H, C₁₋₆alkyl, phenyl or C₁₋₄alkyl substituted with 1 to 3 OH groups; each R³⁶ in Table 1 is independently selected from H, C₁₋₆alkyl, fluoro, benzyloxy substituted with —C(═O)OH, benzyl substituted with —C(═O)OH, C₁₋₄alkoxy substituted with —C(═O)OH and C_{1-A}lkyl substituted with —C(═O)OH; R³⁷ in Table 1 is independently selected from H, phenyl and pyridine, each R⁵ in Table 1 is independently selected from H or C₁₋₆alkyl; R¹² in Table 1 is H, —CH₃ or phenyl; R⁵⁰ in Table 1 is H or nitro.

In some embodiments, a linker component of linker, L, of the compounds disclosed herein is a group formed upon reaction of a reactive functional group with one of the amino acid side chains commonly used for conjugation, e.g., the thiol of cysteine, or the free —NH₂ of lysine, or a Pcl or Pyl group engineered into an antibody. See e.g., Ou, et al., PNAS 108(26), 10437-42 (2011). Linker components formed by reaction with a cysteine residue of the antigen binding moiety include, but are not limited to,

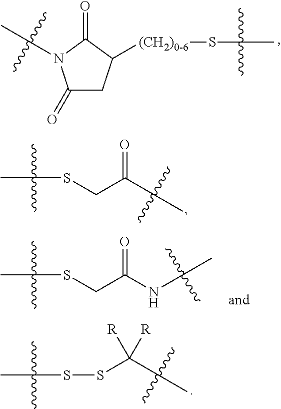

Linker components formed by reaction with the —NH₂ of a lysine residue of the antigen binding moiety, where each p is 1-10, and each R is independently H or C₁₋₄ alkyl (preferably methyl) include, but are not limited to,

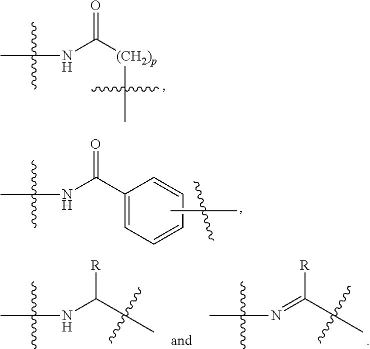

Linker components formed by reaction with a Pcl or Pyl group include, but are not limited to,

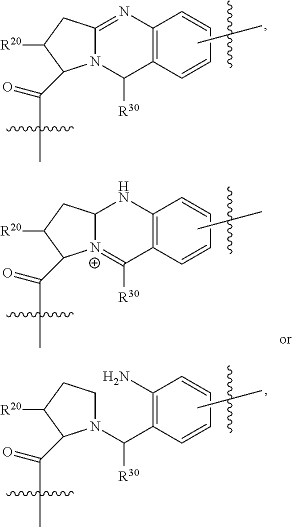

wherein R²⁰ is H or Me, and R³⁰ is H, Me or Phenyl, for linking, where the acyl group shown attaches to the lysine portion of a Pcl or Pyl in an engineered antibody. A linker component formed upon reaction of an Ab disulfide bridge,

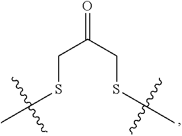 [SMILES: *SCC(=O)CS*]

and a compound of Formula C which contains an hydroxylamine is

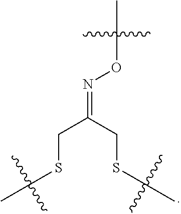 [SMILES: *ON=C(CS*)CS*]

A linker component formed upon reaction of an Ab disulfide bridge,

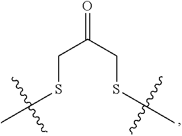 [SMILES: *SCC(=O)CS*]

and a compound of Formula C which contains an hydroxylamine is

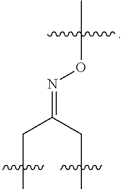 [SMILES: *CC(C*)=NO*]

In some embodiments, a linker component of linker, L, include, for example, alkylene groups —(CH₂)ₙ— (where n is typically 1-10 or 1-6), ethylene glycol units (—CH₂CH₂O—)ₙ (where n is 1-20, typically 1-10 or 1-6), —O—, —S—, carbonyl (—C(═O)—), amides —C(═O)—NH— or —NH—C(═O)—, esters —C(═O)—O— or —O—C(═O)—, ring systems having two available points of attachment such as a divalent ring selected from phenyl (including 1,2-1,3- and 1,4-di-substituted phenyls), C₅₋₆heteroaryl, C₃₋₈ cycloalkyl including 1,1-disubstituted cyclopropyl, cyclobutyl, cyclopentyl or cyclohexyl, and 1,4-disubstituted cyclohexyl, and C₄₋₈ heterocyclyl rings, and specific examples depicted below; amino acids —NH—CHR*—C═O— or —C(═O)—CHR*—NH—, or groups derived from amino acids that attach to N of an adjacent structure (e.g., to a maleimide nitrogen) having the formula [N]—CHR*—C(═O)— where R* is the side chain of a known amino acid (frequently one of the canonical amino acids, e.g., trp, ala, asp, lys, gly, and the like, but also including e.g. norvaline, norleucine, homoserine, homocysteine, phenylglycine, citrulline, and other commonly named alpha-amino acids), polypeptides of known amino acids (e.g., dipeptides, tripeptides, tetrapeptides, etc.), thiol-maleimide linkages (from addition of —SH to maleimide), —S—CR₂— and other thiol ethers such as —S—CR₂—C(═O)— or —C(═O)—CR₂—S— where R is independently at each occurrence H or C₁₋₄ alkyl, —CH₂—C(═O)—, and disulfides (—S—S—), as well as combinations of any of these with other linker components described below, e.g., a bond, a non-enzymatically cleavable linker, a non-cleavable linker, an enzymatically cleavable linker, a photo-stable linker, a photo-cleavable linker or a linker that comprises a self-immolative spacer.

In certain embodiments, Linker, L, is -*L₁L₂L₃L₄L₅L₆-, where the * denotes the site of attachment to the cytotoxic peptide of the invention. In certain embodiments, Linker, L, is -*L₁L₂L₃L₄L₅-, where the * denotes the site of attachment to the cytotoxic peptide of the invention. In certain embodiments, Linker, L, is -*L₁L₂L₃L₄-, where the * denotes the site of attachment to the cytotoxic peptide of the invention. In certain embodiments, Linker, L, is -*L₁L₂L₃-, where the * denotes the site of attachment to the cytotoxic peptide of the invention. In a preferred embodiment Linker, L, is -*L₁L₂-, where the * denotes the site of attachment to the cytotoxic peptide of the invention. In certain embodiments Linker, L, is -L₁-.

Linker component L₁ of immunoconjugates of Formula A and Formula B is selected from —(CH₂)ₘ—, —C(═O)(CH₂)ₘ—, —C(═O)X₁X₂C(═O)(CH₂)ₘ—, —C(═O)X₁X₂C(═O)(CH₂)ₘNR¹²C(═O)(CH₂)ₘ—, —C(═O)X₁X₂C(═O)(CH₂)ₘX₃(CH₂)ₘ—, —C(═O)X₁X₂C(═O)((CH₂)ₘO)ₙ(CH₂)ₘ—, —C(═O)X₁X₂C(═O)((CH₂)ₘO)ₙ(CH₂)ₘNR¹²C(═O)(CH₂)ₘ—, —C(═O)X₁X₂C(═O)((CH₂)ₘO)ₙ(CH₂)ₘNR¹²C(═O)(CH₂)ₘX₃(CH₂)ₘ—, —C(═O)X₁X₂C(═O)((CH₂)ₘO)ₙ(CH₂)ₘX₃(CH₂)ₘ—, —C(═O)X₁X₂C(═O)(CH₂)ₘNR¹²C(═O)((CH₂)ₘO)ₙ(CH₂)ₘ—, —C(═O)X₁X₂C(═O)(CH₂)ₘNR¹²C(═O)((CH₂)ₘO)ₙ(CH₂)ₘX₃(CH₂)ₘ—, —C(═O)X₁X₂(CH₂)ₘX₃(CH₂)ₘ—, —C(═O)X₁X₂((CH₂)ₘO)ₙ(CH₂)ₘ—, —C(═O)X₁X₂((CH₂)ₘO)ₙ(CH₂)ₘNR¹²C(═O)(CH₂)ₘ—, —C(═O)X₁X₂((CH₂)ₘO)ₙ(CH₂)ₘNR¹²C(═O)(CH₂)ₘX₃(CH₂)ₘ—, —C(═O)X₁X₂((CH₂)ₘO)ₙ(CH₂)ₘX₃(CH₂)ₘ—, —C(═O)X₁X₂(CH₂)ₘNR¹²((CH₂)ₘO)ₙ(CH₂)ₘ—, —C(═O)X₁X₂C(═O)(CH₂)ₘNR¹²((CH₂)ₘO)ₙ(CH₂)ₘX₃(CH₂)ₘ—, —(CH₂)ₘNR¹²C(═O)(CH₂)ₘ—, —C(═O)((CH₂)ₘO)ₙ(CH₂)ₘ—, —(CH₂)ₘS(═O)₂((CH₂)ₘO)ₙ(CH₂)ₘ—, —C(═O)(CH₂)ₘNR¹²(CH₂)ₘ—, —C(═O)NR¹²(CH₂)ₘ—, —C(═O)NR¹²(CH₂)ₘX₃(CH₂)ₘ—, —C(═O)NH(CH₂)ₘNR¹²C(═O)X₁X₂C(═O)(CH₂)ₘ—, —C(═O)(CH₂)ₘX₃((CH₂)ₘO)ₙ—, —C(═O)X₁C(═O)NR¹²(CH₂)ₘNR¹²C(═O)(CH₂)ₘ—, —C(═O)X₁C(═O)NR¹²(CH₂)ₘX₃(CH₂)ₘ—, —C(═O)NR¹²(CH₂)ₘNR¹²C(═O)(CH₂)ₘ—, —C(═O)NR¹²(CH₂)ₘNR¹²C(═O)(CH₂)ₘX₃(CH₂)ₘ—,

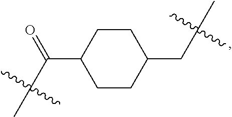 [SMILES: *CC1CCC(C(*)=O)CC1]

—(CH₂)ₘC(═O)NR¹²(CH₂)ₘNR¹²C(═O)(CH₂)ₘ—, —(CH₂)ₘC(═O)—, —(CH₂)ₘC(═O)X₂X₁C(═O)—, —(CH₂)ₘX₃(CH₂)ₘC(═O)X₂X₁C(═O)—, —(CH₂)ₘC(═O)NR¹²(CH₂)ₘ—, —(CH₂)ₘX₃(CH₂)ₘC(═O)NR¹²(CH₂)ₘ—, —(CH₂)ₘNR¹²C(═O)(CH₂)ₘX₃(CH₂)ₘ—, —(CH₂)ₘ(O(CH₂)ₘ)ₙC(═O)—, —(CH₂)ₘ(O(CH₂)ₘ)ₙS(═O)₂(CH₂)ₘ—, —(CH₂)ₘNR¹²(CH₂)ₘC(═O)—, —(CH₂)ₘNR¹²C(═O)—, —(CH₂)ₘC(═O)X₂X₁C(═O)NR¹²(CH₂)ₘNHC(═O)—, —(CH₂)ₘC(═O)NR¹²(CH₂)ₘNR¹²C(═O)X₁—, —(CH₂)ₘC(═O)NR¹²(CH₂)ₘNR¹²C(═O)—,

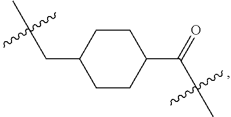 [SMILES: *CC1CCC(C(*)=O)CC1]

—((CH₂)ₘO)ₙ(CH₂)ₘ—, —(CH₂)ₘ(O(CH₂)ₘ)ₙ—, —(CH₂)ₘ(O(CH₂)ₘ)ₙX₃(CH₂)ₘ—, —(CH₂)ₘX₃((CH₂)ₘO)ₙ(CH₂)ₘ—, —(CH₂)ₘX₃(CH₂)ₘC(═O)—, —C(═O)(CH₂)ₘX₃(CH₂)ₘ—, —(CH₂)ₘX₃(CH₂)ₘ(O(CH₂)ₘ)ₙC(═O)—, —C(═O)((CH₂)ₘO)ₙ(CH₂)ₘX₃(CH₂)ₘ—, —(CH₂)ₘC(═O)NR¹²(CH₂)ₘC(═O)—, —C(═O)(CH₂)ₘNR¹²C(═O)(CH₂)ₘ—, —(CH₂)ₘC(═O)NR¹²(CH₂)ₘ(O(CH₂)ₘ)ₙC(═O)—, —C(═O)((CH₂)ₘO)ₙ(CH₂)ₘNR¹²C(═O)(CH₂)ₘ—, —(CH₂)ₘC(═O)NR¹²(CH₂)ₘC(═O)NR¹²(CH₂)ₘ—, —(CH₂)ₘNR¹²C(═O)(CH₂)ₘNR¹²C(═O)(CH₂)ₘ—, —C(═O)NR¹²(CH₂)ₘNR¹²C(═O)—, —(CH₂)ₘS(CH₂)ₘ—, —NR¹²C(═O)(CH₂)ₘ—, —NR¹²C(═O)(CH₂)ₘX₃(CH₂)ₘ—, —(CH₂)ₘX₃(CH₂)ₘC(═O)NR¹²—, —(CH₂)ₘC(═O)NR¹²—, —(CH₂)ₘNR¹²(CH₂)ₘ—, —(CH₂)ₘX₃(CH₂)ₘ—, —((CH₂)ₘO)ₙ(CH₂)ₘX₃(CH₂)ₘ—, —(CH₂)ₘX₃(CH₂)ₘ(O(CH₂)ₘ)ₙ—, —NR¹²(CH₂)ₘ—, —NR¹²C(R¹²)₂(CH₂)ₘ—, —(CH₂)ₘC(R¹²)₂NR¹²—, —(CH₂)ₘC(═O)NR¹²(CH₂)ₘNR¹²—, —NR¹²(CH₂)ₘNR¹²C(═O)(CH₂)ₘ—, —NR¹²C(R¹²)₂(CH₂)ₘNR¹²C(═O)(CH₂)ₘ—, —(CH₂)ₘC(═O)NR¹²(CH₂)ₘC(R¹²)₂NR¹²—, —NR¹²(CH₂)ₘX₃(CH₂)ₘ—, —NR¹²C(R¹²)₂(CH₂)ₘX₃(CH₂)ₘ—, —(CH₂)ₘX₃(CH₂)ₘC(R¹²)₂NR¹²—, —NR¹²C(R¹²)₂(CH₂)ₘOC(═O)NR¹²(CH₂)ₘ—, —(CH₂)ₘNR¹²C(═O)O(CH₂)ₘC(R¹²)₂NR¹²—, —NR¹²C(R¹²)₂(CH₂)ₘOC(═O)NR¹²(CH₂)ₘX₃(CH₂)ₘ—, —(CH₂)ₘX₃(CH₂)ₘNR¹²C(═O)O(CH₂)ₘC(R¹²)₂NR¹²—, —NR¹²C(R¹²)₂(CH₂)ₘOC(═O)NR¹²((CH₂)ₘO)ₙ(CH₂)ₘ—, —(CH₂)ₘ(O(CH₂)ₘ)ₙNR¹²C(═O)O(CH₂)ₘC(R¹²)₂NR¹²—, —NR¹²C(R¹²)₂(CH₂)ₘOC(═O)NR¹²((CH₂)ₘO)ₙ(CH₂)ₘX₃(CH₂)ₘ—, —(CH₂)ₘX₃(CH₂)ₘ(O(CH₂)ₘ)ₙNR¹²C(═O)O(CH₂)ₘC(R¹²)₂NR¹²—, —(CH₂)ₘX₃(CH₂)ₘNR¹²—, —NR¹²((CH₂)ₘO)ₙ(CH₂)ₘX₃(CH₂)ₘ—, —(CH₂)ₘX₃(CH₂)ₘ(O(CH₂)ₘ)ₙNR¹²—, —(CH₂)ₘNR¹²—, —NR¹²((CH₂)ₘO)ₙ(CH₂)ₘ—, —NR¹²((CH₂)ₘO)ₙ(CH₂)ₘNR¹²C(═O)(CH₂)ₘ—, —(CH₂)ₘC(═O)NR¹²(CH₂)ₘ(O(CH₂)ₘ)ₙNR¹² —, —(CH₂)ₘ(O(CH₂)ₘ)ₙNR¹²—, —(C(R₁₂)₂)ₘ—, —(CH₂CH₂O)ₙ—, —(OCH₂CH₂)ₙ—, —(CH₂)ₘO(CH₂)ₘ—, —S(═O)₂(CH₂)ₘ—, —(CH₂)ₘS(═O)₂—, —S(═O)₂(CH₂)ₘNR¹²C(═O)(CH₂)ₘ—, —(CH₂)ₘC(═O)NR¹²(CH₂)ₘS (═O)₂—, —S(═O)₂(CH₂)ₘX₃(CH₂)ₘ—, —(CH₂)ₘX₃(CH₂)ₘS(═O)₂—, —(CH₂)ₘX₂X₁C(═O)—, —C(═O)X₁X₂(CH₂)ₘ—, —(CH₂)ₘ(O(CH₂)ₘ)ₙC(═O)X₂X₁C(═O)—, —C(═O)X₁X₂C(═O)((CH₂)ₘO)ₙ(CH₂)ₘ—, —(CH₂)ₘ(O(CH₂)ₘ)ₙX₂X₁C(═O)—, —(CH₂)ₘX₃(CH₂)ₘX₂X₁C(═O)—, —C(═O)X₁X₂(CH₂)ₘX₃(CH₂)ₘ—, —(CH₂)ₘX₃(CH₂)ₘ(O(CH₂)ₘ)ₙX₂X₁C(═O)—, —(CH₂)ₘX₃(CH₂)ₘC(═O)NR¹²(CH₂)ₘNR¹²C(═O)—, —(CH₂)ₘX₃(CH₂)ₘC(═O)NR¹²(CH₂)ₘC(═O)—, —C(═O)(CH₂)ₘNR¹²C(═O(CH₂)ₘX₃(CH₂)ₘ—, —(CH₂)ₘX₃(CH₂)ₘC(═O)NR¹²(CH₂)ₘ(O(CH₂)ₘ)ₙC(═O)—, —C(═O)((CH₂)ₘO)ₙ(CH₂)ₘNR¹²C(═O)(CH₂)ₘX₃(CH₂)ₘ—, —(CH₂)ₘNR¹²C(═O)X₁X₂C(═O)(CH₂)ₘ—, —(CH₂)ₘC(═O)X₂X₁C(═O)NR¹²(CH₂)ₘ—, —X₄X₁X₂C(═O)(CH₂)ₘ—, —(CH₂)ₘC(═O)X₂X₁X₄—, —X₁C(═O)(CH₂)ₘNHC(═O)(CH₂)ₘ—, —(CH₂)ₘC(═O)NH(CH₂)ₘC(═O)X₁—, —C(═O)CHR^{aa}NR¹²—, —CHR^{aa}C(═O)—, —C(═O)NR¹²—, —C(═O)O—, —S—, —SCH₂(C═O)NR¹²—, —NR¹²C(═O)CH₂S—, —S(═O)₂CH₂CH₂S—, —SCH₂CH₂S(═O)₂—, —(CH₂)₂S(═O)₂CH₂CH₂S—, —SCH₂CH₂S(═O)₂CH₂CH₂—, —NR¹²C(═S)—, —(CH₂)ₘX₃(O(CH₂)ₘ)ₙC(═O)—, —C(═O)((CH₂)ₘO)ₙX₃(CH₂)ₘ—, —(CH₂)ₘNR¹²C(═O)((CH₂)ₘO)ₙ(CH₂)ₘ—, —(CH₂)ₘ(O(CH₂)ₘ)ₙC(═O)NR¹²(CH₂)ₘ—, —(CH₂)ₘNHC(═O)NR¹²(CH₂)ₘ—, —(CH₂)ₘX₃(CH₂)ₘNR¹²C(═O)—, —C(═O)NR¹²(CH₂)ₘX₃(CH₂)ₘ—, —NR₁₂S(═O)₂(CH₂)ₘX₃(CH₂)ₘ—, —(CH₂)ₘX₃(CH₂)ₘS(═O)₂NR₁₂—,

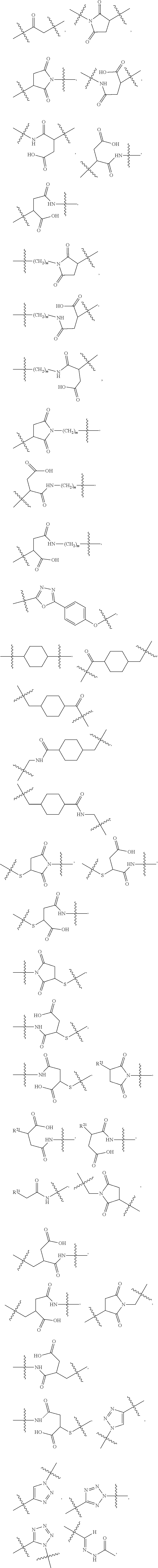

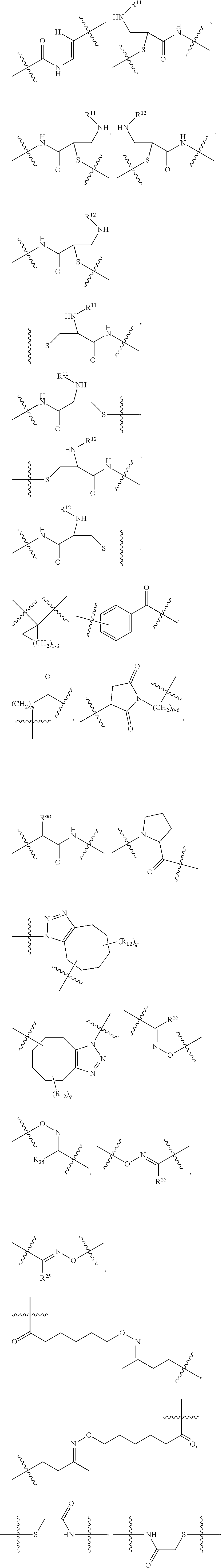

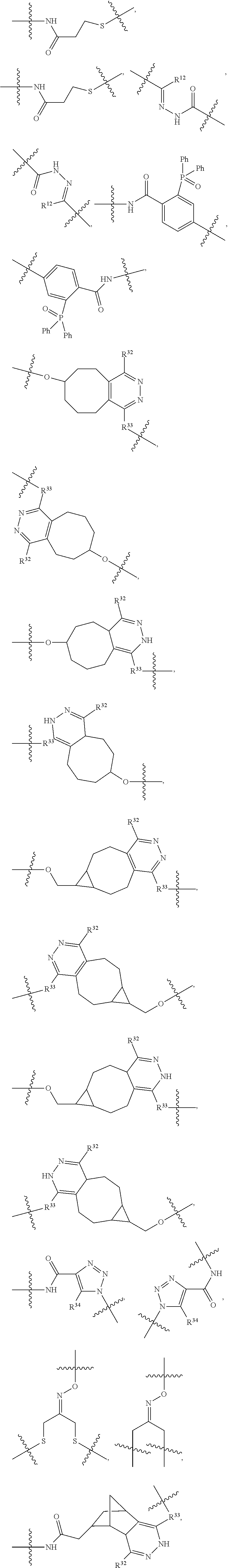

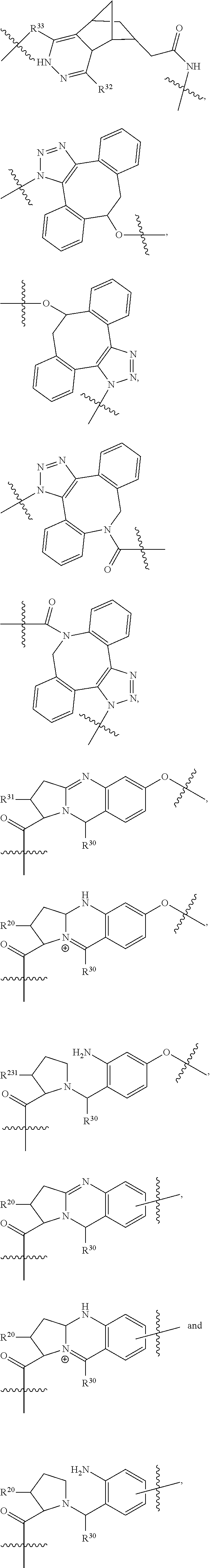

wherein:
R²⁰ is H or Me, and R³⁰ is H, —CH₃ or phenyl;
R²¹ is

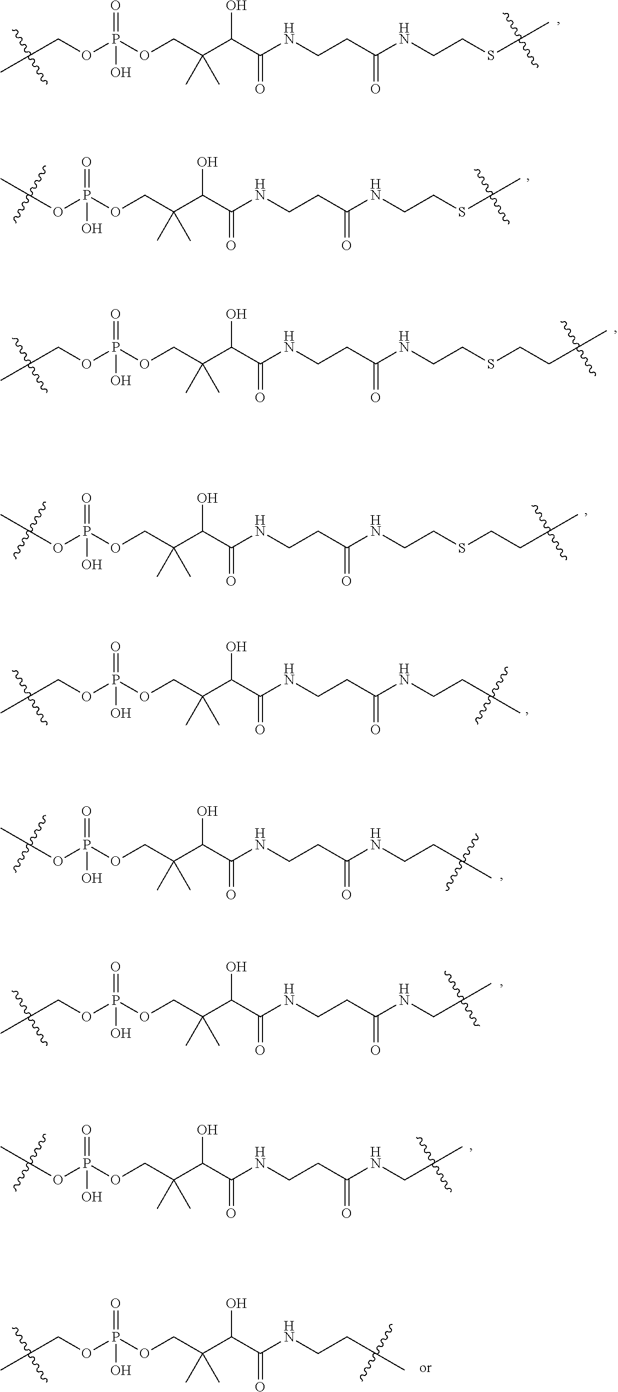

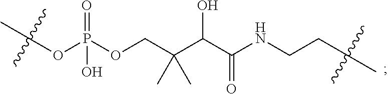 [SMILES: *CCNC(=O)C(O)C(C)(C)COP(=O)(O)O*]

each R²⁵ is independently selected from H or C₁₋₄ alkyl;
R^{aa} is a side chain of an amino acid selected from glycine, alanine, tryptophan, tyrosine, phenylalanine, leucine, isoleucine, valine, asparagine, glutamic acid, glutamine, aspatic acid, histidine, arginine, lysine, cysteine, methionine, serine, threonine, phenylglycine and t-butylglycine;
R³² is independently selected from H, C₁₋₄ alkyl, phenyl, pyrimidine and pyridine;
R³³ is independently selected from

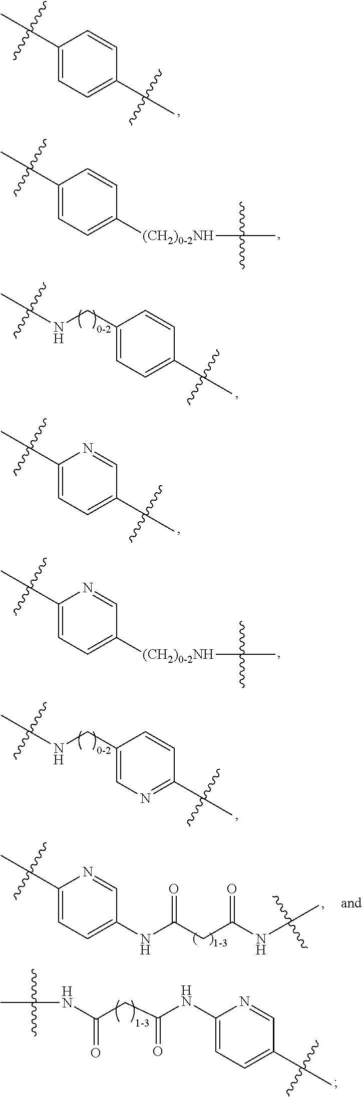

and
R³⁴ is independently selected from H, C₁₋₄ alkyl, and C₁₋₆ haloalkyl;
X₁ is self immolative spacer selected from

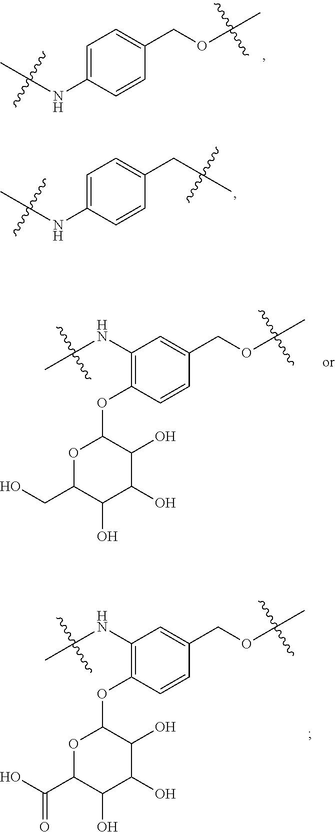 [SMILES: *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is dipeptide selected from

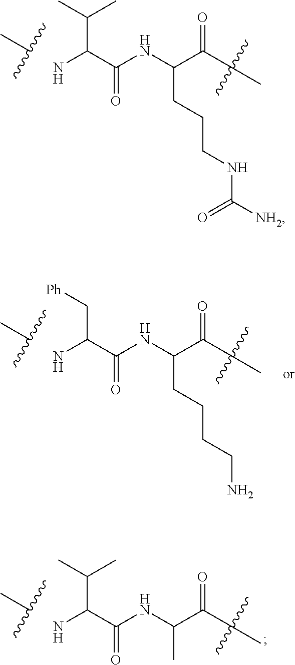

X₃ is

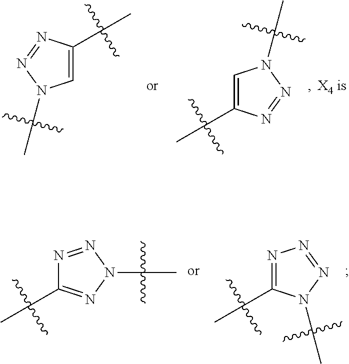 [SMILES: *c1cn(*)nn1 ; *c1cn(*)nn1 ; *c1nnn(*)n1 ; *c1nnnn1*]

each m is independently selected from 1, 2, 3, 4, 5, 6, 7, 8, 9 and 10, and
each n is independently selected from 1, 2, 3, 4, 5, 6, 7, 8, 9, 10, 11, 12, 13, 14, 15, 16,17 and 18.

### 3. Conjugation and Preparation of ADCs

Conjugates of the present invention can be prepared by any methods known in the art, such as those described in U.S. Pat. Nos. 7,811,572, 6,411,163, 7,368,565, and 8,163,888, and patent application publications US2011/0003969, US2011/0166319, US2012/0253021, US2012/0259100, WO2014/0124258, WO2013/0184514, WO2014/0124316, WO2014/083505, and WO2015/079376. The entire teachings of these patents and patent application publications are herein incorporated by reference.

In some embodiments, an immunoconjugate of Formula A or Formula B, or subformulae thereof, comprises an antibody or antibody fragment Ab having antigen-binding activity, where the linker L is attached to Ab at a cysteine sulfur atom of Ab. Typical reactive groups used for reaction with a cysteine sulfur group and the resulting group formed are given in Table 1. Non-limiting examples of linker components formed by reaction with a cysteine residue of the antigen binding moiety include, but are not limited to,

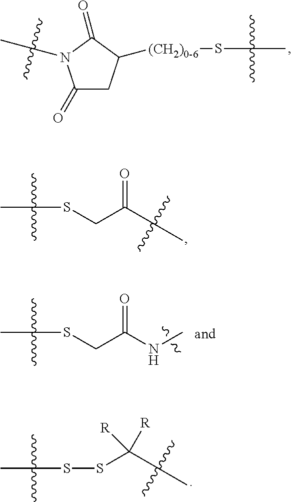

In some embodiments, the Ab has been modified such that one or more cysteine residues have been engineered into non-naturally occurring positions on the heavy chain, the light chain, or both the heavy chain and light chain. By way of example, cysteine modifications to Abs, to enable DAR controlled, site-specific conjugation, are disclosed in WO/2014/124316 and PCT/US2015/019984, which are incorporated herein by reference in their entirety.

In specific embodiments, the Ab has been modified such that cysteine residues have been substituted in place of glutamate at position 152 of the heavy chain (E152C) and serine at position 375 of the heavy chain (S375C), wherein the amino acid positions are numbered according to the EU system. In other specific embodiments, the Ab has been modified such that cysteine residues have been substituted in place of lysine at position 360 of the heavy chain (K360C) and lysine at position 107 of the kappa light chain chain (K107C), wherein the amino acid positions are numbered according to the EU system.

In some embodiments, the linker (or linker-payload) is conjugated to cysteine modified anti-P-cadherin antibodies via a thiol-maleimide linkage at cysteines at positions 152 and 375 of the heavy chains of the antibodies, wherein the amino acid positions are numbered according to the EU system. In other embodiments, the linker (or linker-payload) is conjugated to cysteine modified anti-P-cadherin antibodies via a thiol-maleimide linkage a cysteines at position 360 of the eheavy chain and position 107 of the kappa light chain, wherein the amino acid positions are numbered according to the EU system.

In other embodiments, an immunoconjugate of Formula A or Formula B comprise Ab, an antibody or antibody fragment having antigen-binding activity, where the linker is attached to Ab via a bridged disulfide in Ab. In such embodiments a

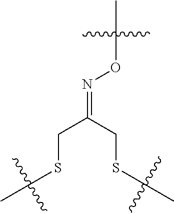 [SMILES: *ON=C(CS*)CS*]

moiety h is formed upon reaction of

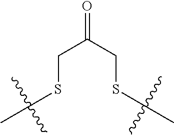 [SMILES: *SCC(=O)CS*]

and a compound of Formula C which contains a hydroxylamine. Such conjugations are described, for example, in WO/2014/083505 and WO/2015/079376, which are incorporated herein by reference in their entirety. These conjugations may be referred to herein at ketone bridge conjugations. The resulting linkage may also be referred to as an oxime linkage.

In some embodiments, an immunoconjugate of Formula A or Formula B, or subformulae thereof, comprises an antibody or antibody fragment Ab having antigen-binding activity, where the linker L is attached to Ab at a free —NH₂ of lysine. The Linker components formed by reaction with the —NH₂ of a lysine residue of the antigen binding moiety, where each p is 1-10, and each R is independently H or C₁₋₄ alkyl (preferably methyl) include, but are not limited to,

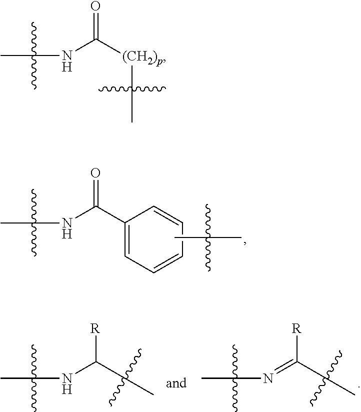

In some embodiments, an immunoconjugate of Formula A or Formula B, or subformulae thereof, comprises an antibody or antibody fragment Ab having antigen-binding activity, where the linker L is attached to Ab at a Pcl or Pyl group engineered into an antibody. See e.g., Ou, et al., PNAS 108(26), 10437-42 (2011). Linker components formed by reaction with a Pc1 or Pyl group include, but are not limited to,

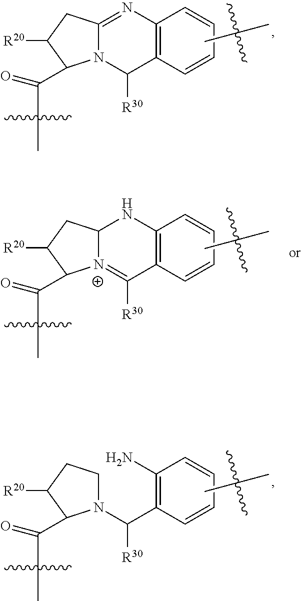

wherein R²⁰ is H or Me, and R³⁰ is H, Me or Phenyl, for linking, where the acyl group shown attaches to the lysine portion of a Pcl or Pyl in an engineered antibody.

In some embodiments, an immunoconjugate of Formula A or Formula B, or subformulae thereof, comprises an antibody or antibody fragment Ab having antigen-binding activity, where the linker L is attached to Ab at serine residue in an S6, ybbR or A1 peptide engineered into an antibody. Linker components formed by reaction with such serine residues include, but are not limited to,

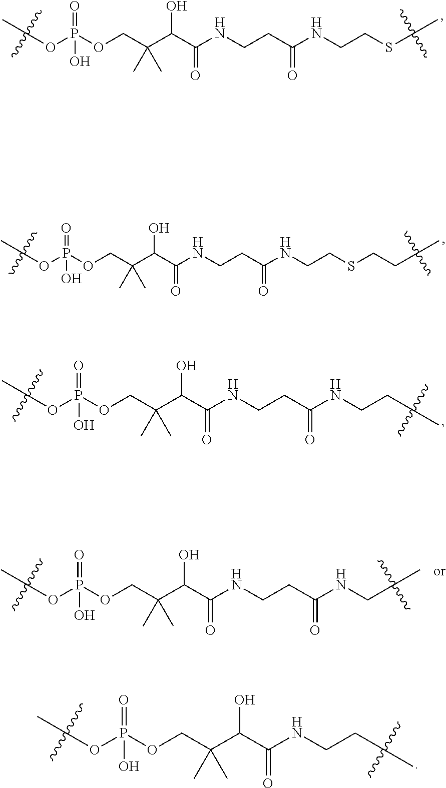 [SMILES: *OP(=O)(O)OCC(C)(C)C(O)C(=O)NCCC(=O)NCCS* ; *CCSCCNC(=O)CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O* ; *CCNC(=O)C(O)C(C)(C)COP(=O)(O)O*]

By way of example, one general reaction scheme for the formation of immunoconjugates of Formula A is shown in Scheme 1 below:

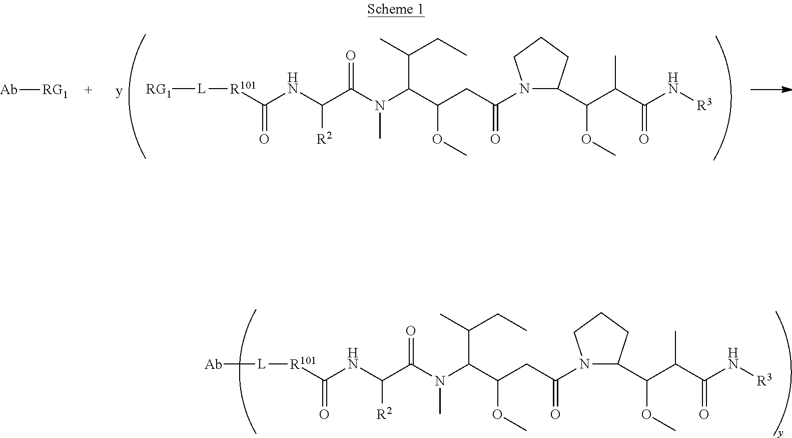

where RG₁ is a reactive group 1 from Table 1 and RG₂ is a reactive group 2 from Table 1 and the reaction product of the respective groups (as seen in Table 1) is a linker component of linker L. R¹⁰¹, R², R³, L and Ab are as defined herein.

Another general reaction scheme for the formation of immunoconjugates of Formula A is shown in Scheme 2 below:

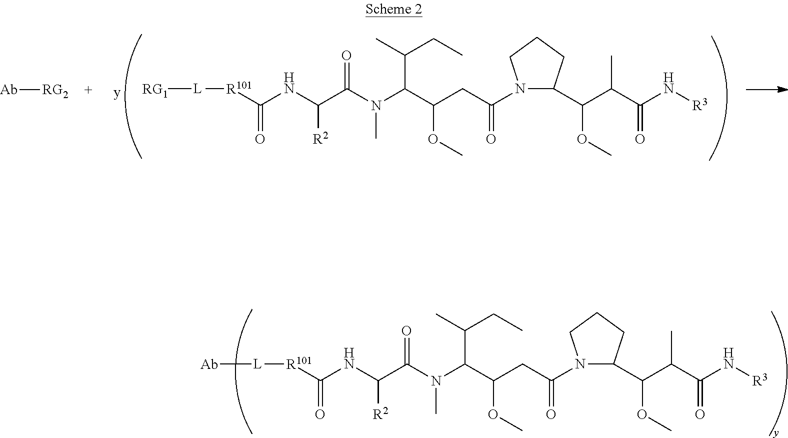

where RG₁ is a reactive group 1 from Table 1 and RG₂ is a reactive group 2 from Table 1 and the reaction product of the respective groups (as seen in Table 1) is a linker component of linker L. R¹⁰¹, R², R³, L and Ab are as defined herein.

By way of example, one general reaction scheme for the formation of immunoconjugates of Formula B is shown in Scheme 3 below:

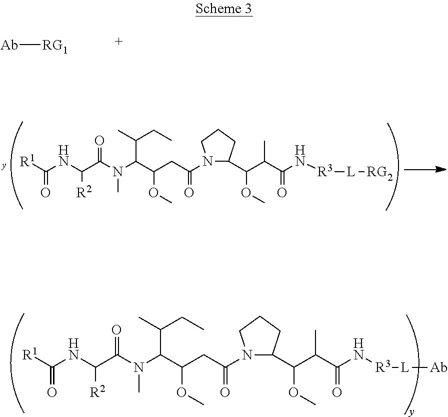

where RG₁ is a reactive group 1 from Table 1 and RG₂ is a reactive group 2 from Table 1 and the reaction product of the respective groups (as seen in Table 1) is a linker component of linker L. R¹, R², R³, L and Ab are as defined herein.

Another general reaction scheme for the formation of immunoconjugates of Formula A is shown in Scheme 4 below:

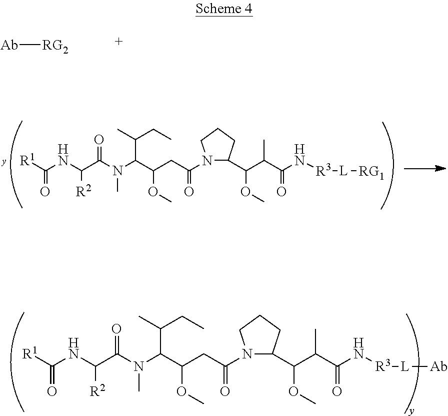

where RG₁ is a reactive group 1 from Table 1 and RG₂ is a reactive group 2 from Table 1 and the reaction product of the respective groups (as seen in Table 1) is a linker component of linker L. R¹, R², R³, L and Ab are as defined herein.

### Anti-P-Cadherin Antibodies

The present invention provides antibodies or antibody fragments (e.g., antigen binding fragments) that specifically bind to human P-cadherin. Antibodies or antibody fragments (e.g., antigen binding fragments) of the invention include, but are not limited to, the human monoclonal antibodies or fragments thereof, isolated as described in the Examples.

The present invention in certain embodiments provides antibodies or antibody fragments (e.g., antigen binding fragments) that specifically bind P-cadherin, said antibodies or antibody fragments (e.g., antigen binding fragments) comprise a VH domain having an amino acid sequence of SEQ ID NO: 7, 27, 47, 67, 87, or 107. The present invention in certain embodiments also provides antibodies or antibody fragments (e.g., antigen binding fragments) that specifically bind to P-cadherin, said antibodies or antibody fragments (e.g., antigen binding fragments) comprise a VH CDR having an amino acid sequence of any one of the VH CDRs listed in Table 2, infra. In particular embodiments, the invention provides antibodies or antibody fragments (e.g., antigen binding fragments) that specifically bind to P-cadherin, said antibodies comprising (or alternatively, consist of) one, two, three, four, five or more VH CDRs having an amino acid sequence of any of the VH CDRs listed in Table 2, infra.

The present invention provides antibodies or antibody fragments (e.g., antigen binding fragments) that specifically bind to P-cadherin, said antibodies or antibody fragments (e.g., antigen binding fragments) comprise a VL domain having an amino acid sequence of SEQ ID NO: 17, 37, 57, 77, 97, or 117. The present invention also provides antibodies or antibody fragments (e.g., antigen binding fragments) that specifically bind to P-cadherin, said antibodies or antibody fragments (e.g., antigen binding fragments) comprise a VL CDR having an amino acid sequence of any one of the VL CDRs listed in Table 2, infra. In particular, the invention provides antibodies or antibody fragments (e.g., antigen binding fragments) that specifically bind to P-cadherin, said antibodies or antibody fragments (e.g., antigen binding fragments) comprise (or alternatively, consist of) one, two, three or more VL CDRs having an amino acid sequence of any of the VL CDRs listed in Table 2, infra.

Other antibodies or antibody fragments (e.g., antigen binding fragments) of the invention include amino acids that have been mutated, yet have at least 60, 70, 80, 90 or 95 percent identity in the CDR regions with the CDR regions depicted in the sequences described in Table 2. In some embodiments, the antibodies comprise mutant amino acid sequences wherein no more than 1, 2, 3, 4 or 5 amino acids have been mutated in the CDR regions when compared with the CDR regions depicted in the sequence described in Table 2.

The present invention also provides anti-P-cadherin antibodies or antigen binding fragments thereof that comprise modifications in the constant regions of the heavy chain, light chain, or both the heavy and light chain wherein particular amino acid residues have mutated to cysteines, also referred to herein at “CysMab” or “Cys” antibodies. As discussed above, drug moieties may be conjugated site specifically and with control over the number of drug moieties (“DAR Controlled”) to cysteine residues on antibodies. Cysteine modifications to antibodies for the purposes of site specifically controlling immunoconjugation are disclosed, for example, in WO2014/124316, which is incorporated herein in its entirety.

In some embodiments, the anti-P-cadherin antibodies have been modified at positions 152 and 375 of the heavy chain, wherein the positions are defined according to the EU numbering system. Namely, the modifications are E152C and S375C. In other embodiments, the anti-P-cadherin antibodies have been modified at position 360 of the heavy chain and position 107 of the kappa light chain, wherein the positions are defined according to the EU numbering system. Namely, the modifications are K360C and K107C. The positions of these mutations are illustrated, for example, in the context of human IgG1 heavy chain and kappy light chain constant regions in SEQ ID NOS:148-150 in Table 2. Throughout Table 2, cysteine modifications from wild type sequences are shown with underlining.

The present invention also provides nucleic acid sequences that encode the VH, VL, the full length heavy chain, and the full length light chain of the antibodies that specifically bind to P-cadherin. Such nucleic acid sequences can be optimized for expression in mammalian cells.

TABLE 2
Examples of anti-P-cadherin Antibodies of the Present Invention
NOV169N31Q

| SEQ ID NO. | Description | Sequence |
|---|---|---|
| SEQ ID NO: 1 | HCDR1 | SQSAAWN |
| | (Kabat) | |
| SEQ ID NO: 2 | HCDR2 | RIYYRSKWYNDYALSVKS |
| | (Kabat) | |
| SEQ ID NO: 3 | HCDR3 | GEGYGREGFAI |
| | (Kabat) | |
| SEQ ID NO: 4 | HCDR1 | GDSVSSQSA |
| | (Chothia) | |
| SEQ ID NO: 5 | HCDR2 | YYRSKWY |
| | (Chothia) | |
| SEQ ID NO: 6 | HCDR3 | GEGYGREGFAI |
| | (Chothia) | |
| SEQ ID NO: 7 | VH | QVQLQQSGPGLVKPSQTLSLTCAISGDSVSSQSAAWNWI |
| | | RQSPSRGLEWLGRIYYRSKWYNDYALSVKSRITINPDTSK |
| | | NQFSLQLNSVTPEDTAVYYCARGEGYGREGFAIWGQGT |
| | | LVTVSS |
| SEQ ID NO: 8 | DNA VH | CAGGTGCAGCTGCAGCAGTCAGGCCCTGGCCTGGTCA |
| | | AGCCTAGTCAGACCCTGAGCCTGACCTGCGCTATTAGC |
| | | GGCGATAGTGTGTCTAGTCAGTCAGCCGCCTGGAACT |
| | | GGATTAGACAGTCACCCTCTAGGGGCCTGGAGTGGCT |
| | | GGGTAGAATCTACTATAGGTCTAAGTGGTATAACGAC |
| | | TACGCCCTGAGCGTGAAGTCTAGGATCACTATTAACCC |
| | | CGACACCTCTAAGAATCAGTTTAGCCTGCAGCTGAATA |
| | | GCGTGACCCCCGAGGACACCGCCGTCTACTACTGCGCT |
| | | AGAGGCGAGGGCTACGGTAGAGAGGGCTTCGCTATCT |
| | | GGGGTCAGGGCACCCTGGTCACCGTGTCTAGC |
| SEQ ID NO: 151 | DNA VH | CAGGTGCAATTGCAGCAGAGCGGTCCGGGCCTGGTGA |
| | | AACCGAGCCAGACCCTGAGCCTGACCTGCGCGATTTC |
| | | CGGAGATAGCGTGAGCTCTCAGTCTGCTGCTTGGAACT |
| | | GGATTCGTCAGAGCCCGAGCCGTGGCCTCGAGTGGCT |
| | | GGGCCGTATCTACTACCGTAGCAAATGGTACAACGAC |
| | | TATGCCTTGAGCGTGAAAAGCCGCATTACCATTAACCC |
| | | GGATACTTCGAAAAACCAGTTTAGCCTGCAACTGAAC |
| | | AGCGTGACCCCGGAAGATACGGCCGTGTATTATTGCG |
| | | CGCGTGGTGAAGGTTACGGTCGTGAAGGTTTCGCTATC |
| | | TGGGGCCAAGGCACCCTGGTGACTGTTAGCTCA |
| SEQ ID NO: 9 | Heavy Chain | QVQLQQSGPGLVKPSQTLSLTCAISGDSVSSQSAAWNWI |
| | | RQSPSRGLEWLGRIYYRSKWYNDYALSVKSRITINPDTSK |
| | | NQFSLQLNSVTPEDTAVYYCARGEGYGREGFAIWGQGT |
| | | LVTVSSASTKGPSVFPLAPSSKSTSGGTAALGCLVKDYFP |
| | | EPVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAKGQPREPQ |
| | | VYTLPPSREEMTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: 10 | DNA Heavy | CAGGTGCAGCTGCAGCAGTCAGGCCCTGGCCTGGTCA |
| | Chain | AGCCTAGTCAGACCCTGAGCCTGACCTGCGCTATTAGC |
| | | GGCGATAGTGTGTCTAGTCAGTCAGCCGCCTGGAACT |
| | | GGATTAGACAGTCACCCTCTAGGGGCCTGGAGTGGCT |
| | | GGGTAGAATCTACTATAGGTCTAAGTGGTATAACGAC |
| | | TACGCCCTGAGCGTGAAGTCTAGGATCACTATTAACCC |
| | | CGACACCTCTAAGAATCAGTTTAGCCTGCAGCTGAATA |
| | | GCGTGACCCCCGAGGACACCGCCGTCTACTACTGCGCT |
| | | AGAGGCGAGGGCTACGGTAGAGAGGGCTTCGCTATCT |
| | | GGGGTCAGGGCACCCTGGTCACCGTGTCTAGCGCTAG |
| | | CACTAAGGGCCCAAGTGTGTTTCCCCTGGCCCCCAGCA |
| | | GCAAGTCTACTTCCGGCGGAACTGCTGCCCTGGGTTGC |
| | | CTGGTGAAGGACTACTTCCCCGAGCCCGTGACAGTGTC |
| | | CTGGAACTCTGGGGCTCTGACTTCCGGCGTGCACACCT |
| | | TCCCCGCCGTGCTGCAGAGCAGCGGCCTGTACAGCCT |
| | | GAGCAGCGTGGTGACAGTGCCCTCCAGCTCTCTGGGA |
| | | ACCCAGACCTATATCTGCAACGTGAACCACAAGCCCA |
| | | GCAACACCAAGGTGGACAAGAGAGTGGAGCCCAAGA |
| | | GCTGCGACAAGACCCACACCTGCCCCCCCTGCCCAGCT |
| | | CCAGAACTGCTGGGAGGGCCTTCCGTGTTCCTGTTCCC |
| | | CCCCAAGCCCAAGGACACCCTGATGATCAGCAGGACC |
| | | CCCGAGGTGACCTGCGTGGTGGTGGACGTGTCCCACG |
| | | AGGACCCAGAGGTGAAGTTCAACTGGTACGTGGACGG |
| | | CGTGGAGGTGCACAACGCCAAGACCAAGCCCAGAGAG |
| | | GAGCAGTACAACAGCACCTACAGGGTGGTGTCCGTGC |
| | | TGACCGTGCTGCACCAGGACTGGCTGAACGGCAAAGA |
| | | ATACAAGTGCAAAGTCTCCAACAAGGCCCTGCCAGCC |
| | | CCAATCGAAAAGACAATCAGCAAGGCCAAGGGCCAGC |
| | | CACGGGAGCCCCAGGTGTACACCCTGCCCCCCAGCCG |
| | | GGAGGAGATGACCAAGAACCAGGTGTCCCTGACCTGT |
| | | CTGGTGAAGGGCTTCTACCCCAGCGATATCGCCGTGG |
| | | AGTGGGAGAGCAACGGCCAGCCCGAGAACAACTACA |
| | | AGACCACCCCCCCAGTGCTGGACAGCGACGGCAGCTT |
| | | CTTCCTGTACAGCAAGCTGACCGTGGACAAGTCCAGG |
| | | TGGCAGCAGGGCAACGTGTTCAGCTGCAGCGTGATGC |
| | | ACGAGGCCCTGCACAACCACTACACCCAGAAGTCCCT |
| | | GAGCCTGAGCCCCGGCAAG |
| SEQ ID NO: 130 | E152C/S375C | QVQLQQSGPGLVKPSQTLSLTCAISGDSVSSQSAAWNWI |
| | CysMab | RQSPSRGLEWLGRIYYRSKWYNDYALSVKSRITINPDTSK |
| | Mutated Heavy | NQFSLQLNSVTPEDTAVYYCARGEGYGREGFAIWGQGT |
| | Chain | LVTVSSASTKGPSVFPLAPSSKSTSGGTAALGCLVKDYFP |
| | | CPVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAKGQPREPQ |
| | | VYTLPPSREEMTKNQVSLTCLVKGFYPCDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: 152 | DNA | CAGGTGCAATTGCAGCAGAGCGGTCCGGGCCTGGTGA |
| | E152C/S375C | AACCGAGCCAGACCCTGAGCCTGACCTGCGCGATTTC |
| | CysMab | CGGAGATAGCGTGAGCTCTCAGTCTGCTGCTTGGAACT |
| | Mutated Heavy | GGATTCGTCAGAGCCCGAGCCGTGGCCTCGAGTGGCT |
| | Chain | GGGCCGTATCTACTACCGTAGCAAATGGTACAACGAC |
| | | TATGCCTTGAGCGTGAAAAGCCGCATTACCATTAACCC |
| | | GGATACTTCGAAAAACCAGTTTAGCCTGCAACTGAAC |
| | | AGCGTGACCCCGGAAGATACGGCCGTGTATTATTGCG |
| | | CGCGTGGTGAAGGTTACGGTCGTGAAGGTTTCGCTATC |
| | | TGGGGCCAAGGCACCCTGGTGACTGTTAGCTCAGCCTC |
| | | TACGAAAGGCCCAAGCGTATTTCCCCTGGCTCCTTCTA |
| | | GTAAATCAACCTCAGGTGGTACAGCAGCCCTTGGCTG |
| | | CCTGGTCAAAGACTATTTCCCCTGTCCGGTGACCGTCT |
| | | CATGGAACTCAGGTGCTTTGACATCTGGTGTGCATACA |
| | | TTCCCAGCTGTGCTGCAAAGTAGTGGACTGTACAGCCT |
| | | TTCCAGCGTGGTCACGGTGCCAAGTAGCTCCTTGGGTA |
| | | CTCAGACTTATATCTGCAATGTGAACCACAAGCCCTCT |
| | | AACACGAAGGTGGACAAGCGCGTGGAGCCCAAATCTT |
| | | GCGATAAGACGCATACTTGTCCCCCATGCCCTGCTCCT |
| | | GAGCTGTTGGGAGGCCCGTCAGTGTTCTTGTTCCCTCC |
| | | GAAGCCTAAGGACACTTTGATGATAAGTAGGACACCA |
| | | GAGGTGACTTGCGTGGTGGTTGATGTGTCCCATGAAG |
| | | ATCCCGAGGTCAAATTTAATTGGTACGTAGATGGTGTC |
| | | GAAGTTCACAATGCTAAGACTAAGCCAAGGGAAGAGC |
| | | AGTACAACAGTACATATAGGGTAGTCTCCGTGCTGAC |
| | | AGTCCTCCACCAGGACTGGTTGAACGGCAAGGAATAC |
| | | AAATGTAAGGTGTCAAACAAAGCTCTGCCTGCTCCCAT |
| | | TGAGAAAACAATCTCTAAAGCCAAAGGCCAGCCGAGA |
| | | GAGCCCCAAGTCTACACTTTGCCCCCGAGCAGGGAGG |
| | | AAATGACCAAGAATCAGGTGAGTCTGACGTGCCTCGT |
| | | CAAAGGATTTTATCCATGCGATATTGCAGTTGAATGGG |
| | | AGAGCAATGGCCAGCCAGAGAACAACTATAAAACCAC |
| | | ACCACCCGTGCTCGACTCTGATGGCAGCTTCTTCCTCT |
| | | ATAGCAAGCTGACAGTCGATAAATCTCGCTGGCAGCA |
| | | AGGCAATGTGTTCTCCTGCTCCGTCATGCACGAGGCTT |
| | | TGCATAACCATTATACTCAAAAATCTCTGTCCCTGTCA |
| | | CCTGGTAAA |
| SEQ ID NO: 131 | K360C | QVQLQQSGPGLVKPSQTLSLTCAISGDSVSSQSAAWNWI |
| | CysMab | RQSPSRGLEWLGRIYYRSKWYNDYALSVKSRITINPDTSK |
| | Mutated Heavy | NQFSLQLNSVTPEDTAVYYCARGEGYGREGFAIWGQGT |
| | Chain | LVTVSSASTKGPSVFPLAPSSKSTSGGTAALGCLVKDYFP |
| | | EPVTVSWNSGALTSGVHTFPAVLQSSGLYSLSSVVTVPSS |
| | | SLGTQTYICNVNHKPSNTKVDKRVEPKSCDKTHTCPPCP |
| | | APELLGGPSVFLFPPKPKDTLMISRTPEVTCVVVDVSHED |
| | | PEVKFNWYVDGVEVHNAKTKPREEQYNSTYRVVSVLTV |
| | | LHQDWLNGKEYKCKVSNKALPAPIEKTISKAKGQPREPQ |
| | | VYTLPPSREEMTCNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| SEQ ID NO: 11 | LCDR1 | RASQTISNTLA |
| | (Kabat) | |
| SEQ ID NO: 12 | LCDR2 | AASNLQS |
| | (Kabat) | |
| SEQ ID NO: 13 | LCDR3 | QQYLSWFT |
| | (Kabat) | |
| SEQ ID NO: 14 | LCDR1 | SQTISNT |
| | (Chothia) | |
| SEQ ID NO: 15 | LCDR2 | AAS |
| | (Chothia) | |
| SEQ ID NO: 16 | LCDR3 | YLSWF |
| | (Chothia) | |
| SEQ ID NO: 17 | VL | DIQMTQSPSSLSASVGDRVTITCRASQTISNTLAWYQQKP |
| | | GKAPKLLIYAASNLQSGVPSRFSGSGSGTDFTLTISSLQPE |
| | | DFATYYCQQYLSWFTFGQGTKVEIK |
| SEQ ID NO: 18 | DNA VL | GATATTCAGATGACTCAGTCACCTAGTAGCCTGAGCGC |
| | | TAGTGTGGGCGATAGAGTGACTATCACCTGTAGAGCC |
| | | TCTCAGACTATCTCTAACACCCTGGCCTGGTATCAGCA |
| | | GAAGCCCGGTAAAGCCCCTAAGCTGCTGATCTACGCC |
| | | GCCTCTAACCTGCAGTCAGGCGTGCCCTCTAGGTTTAG |
| | | CGGTAGCGGTAGTGGCACCGACTTCACCCTGACTATTA |
| | | GTAGCCTGCAGCCCGAGGACTTCGCTACCTACTACTGT |
| | | CAGCAGTACCTGAGCTGGTTCACCTTCGGTCAGGGCAC |
| | | TAAGGTCGAGATTAAG |
| SEQ ID NO: 153 | DNA VL | GATATCCAGATGACCCAGAGCCCGAGCAGCCTGAGCG |
| | | CCAGCGTGGGCGATCGCGTGACCATTACCTGCAGAGC |
| | | CAGCCAGACTATTTCTAACACTCTGGCTTGGTACCAGC |
| | | AGAAACCGGGCAAAGCGCCGAAACTATTAATCTACGC |
| | | TGCTTCTAACCTGCAAAGCGGCGTGCCGAGCCGCTTTA |
| | | GCGGCAGCGGATCCGGCACCGATTTCACCCTGACCATT |
| | | AGCTCTCTGCAACCGGAAGACTTTGCGACCTATTATTG |
| | | CCAGCAGTACCTGTCTTGGTTCACCTTTGGCCAGGGCA |
| | | CGAAAGTTGAAATTAAA |
| SEQ ID NO: 19 | Light Chain | DIQMTQSPSSLSASVGDRVTITCRASQTISNTLAWYQQKP |
| | | GKAPKLLIYAASNLQSGVPSRFSGSGSGTDFTLTISSLQPE |
| | | DFATYYCQQYLSWFTFGQGTKVEIKRTVAAPSVFIFPPSD |
| | | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |
| SEQ ID NO 20: | DNA Light | GATATTCAGATGACTCAGTCACCTAGTAGCCTGAGCGC |
| | Chain | TAGTGTGGGCGATAGAGTGACTATCACCTGTAGAGCC |
| | | TCTCAGACTATCTCTAACACCCTGGCCTGGTATCAGCA |
| | | GAAGCCCGGTAAAGCCCCTAAGCTGCTGATCTACGCC |
| | | GCCTCTAACCTGCAGTCAGGCGTGCCCTCTAGGTTTAG |
| | | CGGTAGCGGTAGTGGCACCGACTTCACCCTGACTATTA |
| | | GTAGCCTGCAGCCCGAGGACTTCGCTACCTACTACTGT |
| | | CAGCAGTACCTGAGCTGGTTCACCTTCGGTCAGGGCAC |
| | | TAAGGTCGAGATTAAGCGTACGGTGGCCGCTCCCAGC |
| | | GTGTTCATCTTCCCCCCCAGCGACGAGCAGCTGAAGA |
| | | GCGGCACCGCCAGCGTGGTGTGCCTGCTGAACAACTT |
| | | CTACCCCCGGGAGGCCAAGGTGCAGTGGAAGGTGGAC |
| | | AACGCCCTGCAGAGCGGCAACAGCCAGGAGAGCGTCA |
| | | CCGAGCAGGACAGCAAGGACTCCACCTACAGCCTGAG |
| | | CAGCACCCTGACCCTGAGCAAGGCCGACTACGAGAAG |
| | | CATAAGGTGTACGCCTGCGAGGTGACCCACCAGGGCC |
| | | TGTCCAGCCCCGTGACCAAGAGCTTCAACAGGGGCGA |
| | | GTGC |
| SEQ ID NO: 154 | DNA Light | GATATCCAGATGACCCAGAGCCCGAGCAGCCTGAGCG |
| | Chain | CCAGCGTGGGCGATCGCGTGACCATTACCTGCAGAGC |
| | | CAGCCAGACTATTTCTAACACTCTGGCTTGGTACCAGC |
| | | AGAAACCGGGCAAAGCGCCGAAACTATTAATCTACGC |
| | | TGCTTCTAACCTGCAAAGCGGCGTGCCGAGCCGCTTTA |
| | | GCGGCAGCGGATCCGGCACCGATTTCACCCTGACCATT |
| | | AGCTCTCTGCAACCGGAAGACTTTGCGACCTATTATTG |
| | | CCAGCAGTACCTGTCTTGGTTCACCTTTGGCCAGGGCA |
| | | CGAAAGTTGAAATTAAACGTACGGTGGCCGCTCCCAG |
| | | CGTGTTCATCTTCCCCCCCAGCGACGAGCAGCTGAAGA |
| | | GCGGCACCGCCAGCGTGGTGTGCCTGCTGAACAACTT |
| | | CTACCCCCGGGAGGCCAAGGTGCAGTGGAAGGTGGAC |
| | | AACGCCCTGCAGAGCGGCAACAGCCAGGAAAGCGTCA |
| | | CCGAGCAGGACAGCAAGGACTCCACCTACAGCCTGAG |
| | | CAGCACCCTGACCCTGAGCAAGGCCGACTACGAGAAG |
| | | CACAAGGTGTACGCCTGCGAGGTGACCCACCAGGGCC |
| | | TGTCCAGCCCCGTGACCAAGAGCTTCAACCGGGGCGA |
| | | GTGT |
| SEQ ID NO: 132 | K107C | DIQMTQSPSSLSASVGDRVTITCRASQTISNTLAWYQQKP |
| | CysMab | GKAPKLLIYAASNLQSGVPSRFSGSGSGTDFTLTISSLQPE |
| | Mutated Light | DFATYYCQQYLSWFTFGQGTKVEICRTVAAPSVFIFPPSD |
| | Chain | EQLKSGTASVVCLLNNFYPREAKVQWKVDNALQSGNSQ |
| | | ESVTEQDSKDSTYSLSSTLTLSKADYEKHKVYACEVTHQ |
| | | GLSSPVTKSFNRGEC |

NEG0012

| | | |
|---|---|---|
| SEQ ID NO: 21 | HCDR1 | DHTIH |
| | (Kabat) | |
| SEQ ID NO: 22 | HCDR2 | YIYPRSGSINYNEKFKG |
| | (Kabat) | |
| SEQ ID NO: 23 | HCDR3 | RNLFLPMEY |
| | (Kabat) | |
| SEQ ID NO: 24 | HCDR1 | GYTFTDH |
| | (Chothia) | |
| SEQ ID NO: 25 | HCDR2 | YPRSGS |
| | (Chothia) | |
| SEQ ID NO: 26 | HCDR3 | RNLFLPMEY |
| | (Chothia) | |
| SEQ ID NO: 27 | VH | EVQLVQSGAEVKKPGESLKISCKVSGYTFTDHTIHWMR |
| | | QMPGKGLEWMGYIYPRSGSINYNEKFKGQVTISADKSS |
| | | STAYLQWSSLKASDTAMYYCARRNLFLPMEYWGQGT |
| | | LVTVSS |
| SEQ ID NO: 28 | DNA VH | GAGGTGCAGCTGGTGCAGTCAGGCGCCGAAGTGAAG |
| | | AAGCCCGGCGAGTCACTGAAGATTAGCTGTAAAGTC |
| | | TCAGGCTACACCTTCACCGATCACACTATTCACTGGA |
| | | TGAGACAGATGCCCGGTAAAGGCCTGGAGTGGATGG |
| | | GCTATATCTACCCTAGATCAGGCTCTATTAACTATAA |
| | | CGAGAAGTTTAAGGGTCAGGTCACAATTAGCGCCGA |
| | | TAAGTCTAGCTCTACCGCCTACCTGCAGTGGTCTAGC |
| | | CTGAAGGCTAGTGACACCGCTATGTACTACTGCGCT |
| | | AGACGTAACCTGTTCCTGCCTATGGAATACTGGGGTC |
| | | AGGGCACCCTGGTCACCGTGTCTAGC |
| SEQ ID NO: 29 | Heavy Chain | EVQLVQSGAEVKKPGESLKISCKVSGYTFTDHTIHWMR |
| | | QMPGKGLEWMGYIYPRSGSINYNEKFKGQVTISADKSS |
| | | STAYLQWSSLKASDTAMYYCARRNLFLPMEYWGQGT |
| | | LVTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 30 | DNA Heavy | GAGGTGCAGCTGGTGCAGTCAGGCGCCGAAGTGAAG |
| | Chain | AAGCCCGGCGAGTCACTGAAGATTAGCTGTAAAGTC |
| | | TCAGGCTACACCTTCACCGATCACACTATTCACTGGA |
| | | TGAGACAGATGCCCGGTAAAGGCCTGGAGTGGATGG |
| | | GCTATATCTACCCTAGATCAGGCTCTATTAACTATAA |
| | | CGAGAAGTTTAAGGGTCAGGTCACAATTAGCGCCGA |
| | | TAAGTCTAGCTCTACCGCCTACCTGCAGTGGTCTAGC |
| | | CTGAAGGCTAGTGACACCGCTATGTACTACTGCGCT |
| | | AGACGTAACCTGTTCCTGCCTATGGAATACTGGGGTC |
| | | AGGGCACCCTGGTCACCGTGTCTAGCGCTAGCACTA |
| | | AGGGCCCAAGTGTGTTTCCCCTGGCCCCCAGCAGCA |
| | | AGTCTACTTCCGGCGGAACTGCTGCCCTGGGTTGCCT |
| | | GGTGAAGGACTACTTCCCCGAGCCCGTGACAGTGTC |
| | | CTGGAACTCTGGGGCTCTGACTTCCGGCGTGCACACC |
| | | TTCCCCGCCGTGCTGCAGAGCAGCGGCCTGTACAGC |
| | | CTGAGCAGCGTGGTGACAGTGCCCTCCAGCTCTCTG |
| | | GGAACCCAGACCTATATCTGCAACGTGAACCACAAG |
| | | CCCAGCAACACCAAGGTGGACAAGAGAGTGGAGCC |
| | | CAAGAGCTGCGACAAGACCCACACCTGCCCCCCCTG |
| | | CCCAGCTCCAGAACTGCTGGGAGGGCCTTCCGTGTTC |
| | | CTGTTCCCCCCCAAGCCCAAGGACACCCTGATGATC |
| | | AGCAGGACCCCCGAGGTGACCTGCGTGGTGGTGGAC |
| | | GTGTCCCACGAGGACCCAGAGGTGAAGTTCAACTGG |
| | | TACGTGGACGGCGTGGAGGTGCACAACGCCAAGACC |
| | | AAGCCCAGAGAGGAGCAGTACAACAGCACCTACAG |
| | | GGTGGTGTCCGTGCTGACCGTGCTGCACCAGGACTG |
| | | GCTGAACGGCAAAGAATACAAGTGCAAAGTCTCCAA |
| | | CAAGGCCCTGCCAGCCCCAATCGAAAAGACAATCAG |
| | | CAAGGCCAAGGGCCAGCCACGGGAGCCCCAGGTGTA |
| | | CACCCTGCCCCCCAGCCGGGAGGAGATGACCAAGAA |
| | | CCAGGTGTCCCTGACCTGTCTGGTGAAGGGCTTCTAC |
| | | CCCAGCGATATCGCCGTGGAGTGGGAGAGCAACGGC |
| | | CAGCCCGAGAACAACTACAAGACCACCCCCCCAGTG |
| | | CTGGACAGCGACGGCAGCTTCTTCCTGTACAGCAAG |
| | | CTGACCGTGGACAAGTCCAGGTGGCAGCAGGGCAAC |
| | | GTGTTCAGCTGCAGCGTGATGCACGAGGCCCTGCAC |
| | | AACCACTACACCCAGAAGTCCCTGAGCCTGAGCCCC |
| | | GGCAAG |
| SEQ ID NO: 133 | E152C/S375C | EVQLVQSGAEVKKPGESLKISCKVSGYTFTDHTIHWMR |
| | CysMab | QMPGKGLEWMGYIYPRSGSINYNEKFKGQVTISADKSS |
| | Mutated | STAYLQWSSLKASDTAMYYCARRNLFLPMEYWGQGT |
| | Heavy Chain | LVTVSSASTKGPSVFPLAPSSKSTSGGTAALGCLVKDYF |
| | | PCPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPCDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 134 | K360C | EVQLVQSGAEVKKPGESLKISCKVSGYTFTDHTIHWMR |
| | CysMab | QMPGKGLEWMGYIYPRSGSINYNEKFKGQVTISADKSS |
| | Mutated | STAYLQWSSLKASDTAMYYCARRNLFLPMEYWGQGT |
| | Heavy Chain | LVTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTCNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 31 | LCDR1 | RSSQSLLSSGDQKNYLT |
| | (Kabat) | |
| SEQ ID NO: 32 | LCDR2 | WASTRES |
| | (Kabat) | |
| SEQ ID NO: 33 | LCDR3 | QNDYRYPLT |
| | (Kabat) | |
| SEQ ID NO: 34 | LCDR1 | SQSLLSSGDQKNY |
| | (Chothia) | |
| SEQ ID NO: 35 | LCDR2 | WAS |
| | (Chothia) | |
| SEQ ID NO: 36 | LCDR3 | DYRYPL |
| | (Chothia) | |
| SEQ ID NO: 37 | VL | DIVMTQTPLSLPVTPGEPASISCRSSQSLLSSGDQKNYLT |
| | | WYLQKPGQSPQLLIYWASTRESGVPDRFSGSGSGTDFT |
| | | LKISRVEAEDVGVYYCQNDYRYPLTFGQGTKLEIK |
| SEQ ID NO: 38 | DNA VL | GATATCGTGATGACTCAGACCCCCCTGAGCCTGCCC |
| | | GTGACCCCTGGCGAGCCTGCCTCTATTAGCTGTAGAT |
| | | CTAGTCAGTCACTGCTGTCTAGCGGCGATCAGAAGA |
| | | ACTACCTGACCTGGTATCTGCAGAAGCCCGGTCAGT |
| | | CACCTCAGCTGCTGATCTACTGGGCCTCTACTAGAGA |
| | | ATCAGGCGTGCCCGATAGGTTTAGCGGTAGCGGTAG |
| | | TGGCACCGACTTCACCCTGAAGATCTCTAGGGTGGA |
| | | AGCCGAGGACGTGGGCGTCTACTACTGTCAGAACGA |
| | | CTATAGATACCCCCTGACCTTCGGTCAGGGCACTAA |
| | | GCTGGAGATTAAG |
| SEQ ID NO: 39 | Light Chain | DIVMTQTPLSLPVTPGEPASISCRSSQSLLSSGDQKNYLT |
| | | WYLQKPGQSPQLLIYWASTRESGVPDRFSGSGSGTDFT |
| | | LKISRVEAEDVGVYYCQNDYRYPLTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| SEQ ID NO 40: | DNA Light | GATATCGTGATGACTCAGACCCCCCTGAGCCTGCCC |
| | Chain | GTGACCCCTGGCGAGCCTGCCTCTATTAGCTGTAGAT |
| | | CTAGTCAGTCACTGCTGTCTAGCGGCGATCAGAAGA |
| | | ACTACCTGACCTGGTATCTGCAGAAGCCCGGTCAGT |
| | | CACCTCAGCTGCTGATCTACTGGGCCTCTACTAGAGA |
| | | ATCAGGCGTGCCCGATAGGTTTAGCGGTAGCGGTAG |
| | | TGGCACCGACTTCACCCTGAAGATCTCTAGGGTGGA |
| | | AGCCGAGGACGTGGGCGTCTACTACTGTCAGAACGA |
| | | CTATAGATACCCCCTGACCTTCGGTCAGGGCACTAA |
| | | GCTGGAGATTAAGCGTACGGTGGCCGCTCCCAGCGT |
| | | GTTCATCTTCCCCCCCAGCGACGAGCAGCTGAAGAG |
| | | CGGCACCGCCAGCGTGGTGTGCCTGCTGAACAACTT |
| | | CTACCCCCGGGAGGCCAAGGTGCAGTGGAAGGTGGA |
| | | CAACGCCCTGCAGAGCGGCAACAGCCAGGAGAGCGT |
| | | CACCGAGCAGGACAGCAAGGACTCCACCTACAGCCT |
| | | GAGCAGCACCCTGACCCTGAGCAAGGCCGACTACGA |
| | | GAAGCATAAGGTGTACGCCTGCGAGGTGACCCACCA |
| | | GGGCCTGTCCAGCCCCGTGACCAAGAGCTTCAACAG |
| | | GGGCGAGTGC |
| SEQ ID NO: 135 | K107C | DIVMTQTPLSLPVTPGEPASISCRSSQSLLSSGDQKNYLT |
| | CysMab | WYLQKPGQSPQLLIYWASTRESGVPDRFSGSGSGTDFT |
| | Mutated Light | LKISRVEAEDVGVYYCQNDYRYPLTFGQGTKLEICRTV |
| | Chain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

NEG0013

| | | |
|---|---|---|
| SEQ ID NO: 41 | HCDR1 | DHTIH |
| | (Kabat) | |
| SEQ ID NO: 42 | HCDR2 | YIYPRSGSINYNEKFKG |
| | (Kabat) | |
| SEQ ID NO: 43 | HCDR3 | RNLFLPMEY |
| | (Kabat) | |
| SEQ ID NO: 44 | HCDR1 | GYTFTDH |
| | (Chothia) | |
| SEQ ID NO: 45 | HCDR2 | YPRSGS |
| | (Chothia) | |
| SEQ ID NO: 46 | HCDR3 | RNLFLPMEY |
| | (Chothia) | |
| SEQ ID NO: 47 | VH | EVQLVQSGAEVKKPGESLKISCKVSGYTFTDHTIHWMR |
| | | QMPGKGLEWMGYIYPRSGSINYNEKFKGQVTISADKSS |
| | | STAYLQWSSLKASDTAMYYCARRNLFLPMEYWGQGT |
| | | LVTVSS |
| SEQ ID NO: 48 | DNA VH | GAGGTGCAGCTGGTGCAGTCAGGCGCCGAAGTGAAG |
| | | AAGCCCGGCGAGTCACTGAAGATTAGCTGTAAAGTC |
| | | TCAGGCTACACCTTCACCGATCACACTATTCACTGGA |
| | | TGAGACAGATGCCCGGTAAAGGCCTGGAGTGGATGG |
| | | GCTATATCTACCCTAGATCAGGCTCTATTAACTATAA |
| | | CGAGAAGTTTAAGGGTCAGGTCACAATTAGCGCCGA |
| | | TAAGTCTAGCTCTACCGCCTACCTGCAGTGGTCTAGC |
| | | CTGAAGGCTAGTGACACCGCTATGTACTACTGCGCT |
| | | AGACGTAACCTGTTCCTGCCTATGGAATACTGGGGTC |
| | | AGGGCACCCTGGTCACCGTGTCTAGC |
| SEQ ID NO: 49 | Heavy Chain | EVQLVQSGAEVKKPGESLKISCKVSGYTFTDHTIHWMR |
| | | QMPGKGLEWMGYIYPRSGSINYNEKFKGQVTISADKSS |
| | | STAYLQWSSLKASDTAMYYCARRNLFLPMEYWGQGT |
| | | LVTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 50 | DNA Heavy | GAGGTGCAGCTGGTGCAGTCAGGCGCCGAAGTGAAG |
| | Chain | AAGCCCGGCGAGTCACTGAAGATTAGCTGTAAAGTC |
| | | TCAGGCTACACCTTCACCGATCACACTATTCACTGGA |
| | | TGAGACAGATGCCCGGTAAAGGCCTGGAGTGGATGG |
| | | GCTATATCTACCCTAGATCAGGCTCTATTAACTATAA |
| | | CGAGAAGTTTAAGGGTCAGGTCACAATTAGCGCCGA |
| | | TAAGTCTAGCTCTACCGCCTACCTGCAGTGGTCTAGC |
| | | CTGAAGGCTAGTGACACCGCTATGTACTACTGCGCT |
| | | AGACGTAACCTGTTCCTGCCTATGGAATACTGGGGTC |
| | | AGGGCACCCTGGTCACCGTGTCTAGCGCTAGCACTA |
| | | AGGGCCCAAGTGTGTTTCCCCTGGCCCCCAGCAGCA |
| | | AGTCTACTTCCGGCGGAACTGCTGCCCTGGGTTGCCT |
| | | GGTGAAGGACTACTTCCCCGAGCCCGTGACAGTGTC |
| | | CTGGAACTCTGGGGCTCTGACTTCCGGCGTGCACACC |
| | | TTCCCCGCCGTGCTGCAGAGCAGCGGCCTGTACAGC |
| | | CTGAGCAGCGTGGTGACAGTGCCCTCCAGCTCTCTG |
| | | GGAACCCAGACCTATATCTGCAACGTGAACCACAAG |
| | | CCCAGCAACACCAAGGTGGACAAGAGAGTGGAGCC |
| | | CAAGAGCTGCGACAAGACCCACACCTGCCCCCCCTG |
| | | CCCAGCTCCAGAACTGCTGGGAGGGCCTTCCGTGTTC |
| | | CTGTTCCCCCCCAAGCCCAAGGACACCCTGATGATC |
| | | AGCAGGACCCCCGAGGTGACCTGCGTGGTGGTGGAC |
| | | GTGTCCCACGAGGACCCAGAGGTGAAGTTCAACTGG |
| | | TACGTGGACGGCGTGGAGGTGCACAACGCCAAGACC |
| | | AAGCCCAGAGAGGAGCAGTACAACAGCACCTACAG |
| | | GGTGGTGTCCGTGCTGACCGTGCTGCACCAGGACTG |
| | | GCTGAACGGCAAAGAATACAAGTGCAAAGTCTCCAA |
| | | CAAGGCCCTGCCAGCCCCAATCGAAAAGACAATCAG |
| | | CAAGGCCAAGGGCCAGCCACGGGAGCCCCAGGTGTA |
| | | CACCCTGCCCCCCAGCCGGGAGGAGATGACCAAGAA |
| | | CCAGGTGTCCCTGACCTGTCTGGTGAAGGGCTTCTAC |
| | | CCCAGCGATATCGCCGTGGAGTGGGAGAGCAACGGC |
| | | CAGCCCGAGAACAACTACAAGACCACCCCCCCAGTG |
| | | CTGGACAGCGACGGCAGCTTCTTCCTGTACAGCAAG |
| | | CTGACCGTGGACAAGTCCAGGTGGCAGCAGGGCAAC |
| | | GTGTTCAGCTGCAGCGTGATGCACGAGGCCCTGCAC |
| | | AACCACTACACCCAGAAGTCCCTGAGCCTGAGCCCC |
| | | GGCAAG |
| SEQ ID NO: 136 | E152C/S375C | EVQLVQSGAEVKKPGESLKISCKVSGYTFTDHTIHWMR |
| | CysMab | QMPGKGLEWMGYIYPRSGSINYNEKFKGQVTISADKSS |
| | Mutated | STAYLQWSSLKASDTAMYYCARRNLFLPMEYWGQGT |
| | Heavy Chain | LVTVSSASTKGPSVFPLAPSSKSTSGGTAALGCLVKDYF |
| | | PCPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTKNQVSLTCLVKGFYPCDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 137 | K360C | EVQLVQSGAEVKKPGESLKISCKVSGYTFTDHTIHWMR |
| | CysMab | QMPGKGLEWMGYIYPRSGSINYNEKFKGQVTISADKSS |
| | Mutated | STAYLQWSSLKASDTAMYYCARRNLFLPMEYWGQGT |
| | Heavy Chain | LVTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKRVEPKSCDKTHTC |
| | | PPCPAPELLGGPSVFLFPPKPKDTLMISRTPEVTCVVVD |
| | | VSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSREEMTCNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| SEQ ID NO: 51 | LCDR1 | RSSQSLLSSGNQKNYLT |
| | (Kabat) | |
| SEQ ID NO: 52 | LCDR2 | WASTRES |
| | (Kabat) | |
| SEQ ID NO: 53 | LCDR3 | QNDYSYPLT |
| | (Kabat) | |
| SEQ ID NO: 54 | LCDR1 | SQSLLSSGNQKNY |
| | (Chothia) | |
| SEQ ID NO: 55 | LCDR2 | WAS |
| | (Chothia) | |
| SEQ ID NO: 56 | LCDR3 | DYSYPL |
| | (Chothia) | |
| SEQ ID NO: 57 | VL | DIVMTQTPLSLPVTPGEPASISCRSSQSLLSSGNQKNYLT |
| | | WYLQKPGQSPQLLIYWASTRESGVPDRFSGSGSGTDFT |
| | | LKISRVEAEDVGVYYCQNDYSYPLTFGQGTKLEIK |
| SEQ ID NO: 58 | DNA VL | GATATCGTGATGACTCAGACCCCCCTGAGCCTGCCC |
| | | GTGACCCCTGGCGAGCCTGCCTCTATTAGCTGTAGAT |
| | | CTAGTCAGTCACTGCTGTCTAGCGGTAATCAGAAGA |
| | | ACTACCTGACCTGGTATCTGCAGAAGCCCGGTCAGT |
| | | CACCTCAGCTGCTGATCTACTGGGCCTCTACTAGAGA |
| | | ATCAGGCGTGCCCGATAGGTTTAGCGGTAGCGGTAG |
| | | TGGCACCGACTTCACCCTGAAGATCTCTAGGGTGGA |
| | | AGCCGAGGACGTGGGCGTCTACTACTGTCAGAACGA |
| | | CTATAGCTACCCCCTGACCTTCGGTCAGGGCACTAAG |
| | | CTGGAGATTAAG |
| SEQ ID NO: 59 | Light Chain | DIVMTQTPLSLPVTPGEPASISCRSSQSLLSSGNQKNYLT |
| | | WYLQKPGQSPQLLIYWASTRESGVPDRFSGSGSGTDFT |
| | | LKISRVEAEDVGVYYCQNDYSYPLTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| SEQ ID NO 60: | DNA Light | GATATCGTGATGACTCAGACCCCCCTGAGCCTGCCC |
| | Chain | GTGACCCCTGGCGAGCCTGCCTCTATTAGCTGTAGAT |
| | | CTAGTCAGTCACTGCTGTCTAGCGGTAATCAGAAGA |
| | | ACTACCTGACCTGGTATCTGCAGAAGCCCGGTCAGT |
| | | CACCTCAGCTGCTGATCTACTGGGCCTCTACTAGAGA |
| | | ATCAGGCGTGCCCGATAGGTTTAGCGGTAGCGGTAG |
| | | TGGCACCGACTTCACCCTGAAGATCTCTAGGGTGGA |
| | | AGCCGAGGACGTGGGCGTCTACTACTGTCAGAACGA |
| | | CTATAGCTACCCCCTGACCTTCGGTCAGGGCACTAAG |
| | | CTGGAGATTAAGCGTACGGTGGCCGCTCCCAGCGTG |
| | | TTCATCTTCCCCCCCAGCGACGAGCAGCTGAAGAGC |
| | | GGCACCGCCAGCGTGGTGTGCCTGCTGAACAACTTC |
| | | TACCCCCGGGAGGCCAAGGTGCAGTGGAAGGTGGAC |
| | | AACGCCCTGCAGAGCGGCAACAGCCAGGAGAGCGTC |
| | | ACCGAGCAGGACAGCAAGGACTCCACCTACAGCCTG |
| | | AGCAGCACCCTGACCCTGAGCAAGGCCGACTACGAG |
| | | AAGCATAAGGTGTACGCCTGCGAGGTGACCCACCAG |
| | | GGCCTGTCCAGCCCCGTGACCAAGAGCTTCAACAGG |
| | | GGCGAGTGC |
| SEQ ID NO:138 | K107C | DIVMTQTPLSLPVTPGEPASISCRSSQSLLSSGNQKNYLT |
| | CysMab | WYLQKPGQSPQLLIYWASTRESGVPDRFSGSGSGTDFT |
| | Mutated Light | LKISRVEAEDVGVYYCQNDYSYPLTFGQGTKLEICRTV |
| | Chain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

NEG0016

| | | |
|---|---|---|
| SEQ ID NO: 61 | HCDR1 | DHTLH |
| | (Kabat) | |
| SEQ ID NO: 62 | HCDR2 | YIYPRSGSTKYNENFRG |
| | (Kabat) | |
| SEQ ID NO: 63 | HCDR3 | RLLFLPLDY |
| | (Kabat) | |
| SEQ ID NO: 64 | HCDR1 | GYTFTDH |
| | (Chothia) | |
| SEQ ID NO: 65 | HCDR2 | YPRSGS |
| | (Chothia) | |
| SEQ ID NO: 66 | HCDR3 | RLLFLPLDY |
| | (Chothia) | |
| SEQ ID NO: 67 | VH | QIQLVQSGAEVKKPGSSVKVSCKVSGYTFTDHTLHWM |
| | | RQAPGQGLEWMGYIYPRSGSTKYNENFRGRVTITADTS |
| | | SSTAYMELSSLRSEDTAVYYCARRLLFLPLDYWGQGTL |
| | | VTVSS |
| SEQ ID NO: 68 | DNA VH | CAGATTCAGCTGGTGCAGTCAGGCGCCGAAGTGAAG |
| | | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAGGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGCT |
| | | AGACGGCTGCTGTTCCTGCCCCTGGACTACTGGGGTC |
| | | AGGGCACCCTGGTCACCGTGTCTAGC |
| SEQ ID NO: 69 | Heavy Chain | QIQLVQSGAEVKKPGSSVKVSCKVSGYTFTDHTLHWM |
| | | RQAPGQGLEWMGYIYPRSGSTKYNENFRGRVTITADTS |
| | | SSTAYMELSSLRSEDTAVYYCARRLLFLPLDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 70 | DNA Heavy | CAGATTCAGCTGGTGCAGTCAGGCGCCGAAGTGAAG |
| | Chain | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAGGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGCT |
| | | AGACGGCTGCTGTTCCTGCCCCTGGACTACTGGGGTC |
| | | AGGGCACCCTGGTCACCGTGTCTAGCGCTAGCACTA |
| | | AGGGCCCAAGTGTGTTTCCCCTGGCCCCCAGCAGCA |
| | | AGTCTACTTCCGGCGGAACTGCTGCCCTGGGTTGCCT |
| | | GGTGAAGGACTACTTCCCCGAGCCCGTGACAGTGTC |
| | | CTGGAACTCTGGGGCTCTGACTTCCGGCGTGCACACC |
| | | TTCCCCGCCGTGCTGCAGAGCAGCGGCCTGTACAGC |
| | | CTGAGCAGCGTGGTGACAGTGCCCTCCAGCTCTCTG |
| | | GGAACCCAGACCTATATCTGCAACGTGAACCACAAG |
| | | CCCAGCAACACCAAGGTGGACAAGAGAGTGGAGCC |
| | | CAAGAGCTGCGACAAGACCCACACCTGCCCCCCCTG |
| | | CCCAGCTCCAGAACTGCTGGGAGGGCCTTCCGTGTTC |
| | | CTGTTCCCCCCCAAGCCCAAGGACACCCTGATGATC |
| | | AGCAGGACCCCCGAGGTGACCTGCGTGGTGGTGGAC |
| | | GTGTCCCACGAGGACCCAGAGGTGAAGTTCAACTGG |
| | | TACGTGGACGGCGTGGAGGTGCACAACGCCAAGACC |
| | | AAGCCCAGAGAGGAGCAGTACAACAGCACCTACAG |
| | | GGTGGTGTCCGTGCTGACCGTGCTGCACCAGGACTG |
| | | GCTGAACGGCAAAGAATACAAGTGCAAAGTCTCCAA |
| | | CAAGGCCCTGCCAGCCCCAATCGAAAAGACAATCAG |
| | | CAAGGCCAAGGGCCAGCCACGGGAGCCCCAGGTGTA |
| | | CACCCTGCCCCCCAGCCGGGAGGAGATGACCAAGAA |
| | | CCAGGTGTCCCTGACCTGTCTGGTGAAGGGCTTCTAC |
| | | CCCAGCGATATCGCCGTGGAGTGGGAGAGCAACGGC |
| | | CAGCCCGAGAACAACTACAAGACCACCCCCCCAGTG |
| | | CTGGACAGCGACGGCAGCTTCTTCCTGTACAGCAAG |
| | | CTGACCGTGGACAAGTCCAGGTGGCAGCAGGGCAAC |
| | | GTGTTCAGCTGCAGCGTGATGCACGAGGCCCTGCAC |
| | | AACCACTACACCCAGAAGTCCCTGAGCCTGAGCCCC |
| | | GGCAAG |
| SEQ ID NO: 139 | E152C/S375C | QIQLVQSGAEVKKPGSSVKVSCKVSGYTFTDHTLHWM |
| | CysMab | RQAPGQGLEWMGYIYPRSGSTKYNENFRGRVTITADTS |
| | Mutated Heavy | SSTAYMELSSLRSEDTAVYYCARRLLFLPLDYWGQGTL |
| | Chain | VTVSSASTKGPSVFPLAPSSKSTSGGTAALGCLVKDYFP |
| | | CPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPCDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO:140 | K360C | QIQLVQSGAEVKKPGSSVKVSCKVSGYTFTDHTLHWM |
| | CysMab | RQAPGQGLEWMGYIYPRSGSTKYNENFRGRVTITADTS |
| | Mutated Heavy | SSTAYMELSSLRSEDTAVYYCARRLLFLPLDYWGQGTL |
| | Chain | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTCNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 71 | LCDR1 | RSSQSLLSSGNQKSYLT |
| | (Kabat) | |
| SEQ ID NO: 72 | LCDR2 | WASTRES |
| | (Kabat) | |
| SEQ ID NO: 73 | LCDR3 | QNDYSYPFT |
| | (Kabat) | |
| SEQ ID NO: 74 | LCDR1 | SQSLLSSGNQKSY |
| | (Chothia) | |
| SEQ ID NO: 75 | LCDR2 | WAS |
| | (Chothia) | |
| SEQ ID NO: 76 | LCDR3 | DYSYPF |
| | (Chothia) | |
| SEQ ID NO: 77 | VL | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIK |
| SEQ ID NO: 78 | DNA VL | GAGATCGTGATGACTCAGTCACCCGCTACCCTGAGC |
| | | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAG |
| SEQ ID NO: 79 | Light Chain | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| SEQ ID NO 80: | DNA Light | GAGATCGTGATGACTCAGTCACCCGCTACCCTGAGC |
| | Chain | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAGCGTACGGTGGCCGCTCCCAGCGTGTT |
| | | CATCTTCCCCCCCAGCGACGAGCAGCTGAAGAGCGG |
| | | CACCGCCAGCGTGGTGTGCCTGCTGAACAACTTCTAC |
| | | CCCCGGGAGGCCAAGGTGCAGTGGAAGGTGGACAA |
| | | CGCCCTGCAGAGCGGCAACAGCCAGGAGAGCGTCAC |
| | | CGAGCAGGACAGCAAGGACTCCACCTACAGCCTGAG |
| | | CAGCACCCTGACCCTGAGCAAGGCCGACTACGAGAA |
| | | GCATAAGGTGTACGCCTGCGAGGTGACCCACCAGGG |
| | | CCTGTCCAGCCCCGTGACCAAGAGCTTCAACAGGGG |
| | | CGAGTGC |
| SEQ ID NO: 141 | K107C | EIVMTQSPATLSLSPGERATLSCRSSQSLLSSGNQKSYL |
| | CysMab | TWYQQKPGQAPRLLIYWASTRESGIPARFSGSGSGTDF |
| | Mutated Light | TLTISSLQPEDFAVYYCQNDYSYPFTFGQGTKLEICRTV |
| | Chain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

NEG0064

| | | |
|---|---|---|
| SEQ ID NO: 81 | HCDR1 | DHTLH |
| | (Kabat) | |
| SEQ ID NO: 82 | HCDR2 | YIYPRSGSTKYNENFRG |
| | (Kabat) | |
| SEQ ID NO: 83 | HCDR3 | RLLFLPLDY |
| | (Kabat) | |
| SEQ ID NO: 84 | HCDR1 | GYTFTDH |
| | (Chothia) | |
| SEQ ID NO: 85 | HCDR2 | YPRSGS |
| | (Chothia) | |
| SEQ ID NO: 86 | HCDR3 | RLLFLPLDY |
| | (Chothia) | |
| SEQ ID NO: 87 | VH | QIQLVQSGAEVKKPGSSVKVSCKVSGYTFTDHTLHWM |
| | | RQAPGQGLEWMGYIYPRSGSTKYNENFRGRVTITADTS |
| | | SSTAYMELSSLRSEDTAVYYCVRRLLFLPLDYWGQGTL |
| | | VTVSS |
| SEQ ID NO: 88 | DNA VH | CAGATTCAGCTGGTGCAGTCAGGCGCCGAAGTGAAG |
| | | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAGGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGTC |
| | | AGACGGCTGCTGTTCCTGCCCCTGGACTACTGGGGTC |
| | | AGGGCACCCTGGTCACCGTGTCTAGC |
| SEQ ID NO: 89 | Heavy Chain | QIQLVQSGAEVKKPGSSVKVSCKVSGYTFTDHTLHWM |
| | | RQAPGQGLEWMGYIYPRSGSTKYNENFRGRVTITADTS |
| | | SSTAYMELSSLRSEDTAVYYCVRRLLFLPLDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 90 | DNA Heavy | CAGATTCAGCTGGTGCAGTCAGGCGCCGAAGTGAAG |
| | Chain | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAGGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGTC |
| | | AGACGGCTGCTGTTCCTGCCCCTGGACTACTGGGGTC |
| | | AGGGCACCCTGGTCACCGTGTCTAGCGCTAGCACTA |
| | | AGGGCCCAAGTGTGTTTCCCCTGGCCCCCAGCAGCA |
| | | AGTCTACTTCCGGCGGAACTGCTGCCCTGGGTTGCCT |
| | | GGTGAAGGACTACTTCCCCGAGCCCGTGACAGTGTC |
| | | CTGGAACTCTGGGGCTCTGACTTCCGGCGTGCACACC |
| | | TTCCCCGCCGTGCTGCAGAGCAGCGGCCTGTACAGC |
| | | CTGAGCAGCGTGGTGACAGTGCCCTCCAGCTCTCTG |
| | | GGAACCCAGACCTATATCTGCAACGTGAACCACAAG |
| | | CCCAGCAACACCAAGGTGGACAAGAGAGTGGAGCC |
| | | CAAGAGCTGCGACAAGACCCACACCTGCCCCCCCTG |
| | | CCCAGCTCCAGAACTGCTGGGAGGGCCTTCCGTGTTC |
| | | CTGTTCCCCCCCAAGCCCAAGGACACCCTGATGATC |
| | | AGCAGGACCCCCGAGGTGACCTGCGTGGTGGTGGAC |
| | | GTGTCCCACGAGGACCCAGAGGTGAAGTTCAACTGG |
| | | TACGTGGACGGCGTGGAGGTGCACAACGCCAAGACC |
| | | AAGCCCAGAGAGGAGCAGTACAACAGCACCTACAG |
| | | GGTGGTGTCCGTGCTGACCGTGCTGCACCAGGACTG |
| | | GCTGAACGGCAAAGAATACAAGTGCAAAGTCTCCAA |
| | | CAAGGCCCTGCCAGCCCCAATCGAAAAGACAATCAG |
| | | CAAGGCCAAGGGCCAGCCACGGGAGCCCCAGGTGTA |
| | | CACCCTGCCCCCCAGCCGGGAGGAGATGACCAAGAA |
| | | CCAGGTGTCCCTGACCTGTCTGGTGAAGGGCTTCTAC |
| | | CCCAGCGATATCGCCGTGGAGTGGGAGAGCAACGGC |
| | | CAGCCCGAGAACAACTACAAGACCACCCCCCCAGTG |
| | | CTGGACAGCGACGGCAGCTTCTTCCTGTACAGCAAG |
| | | CTGACCGTGGACAAGTCCAGGTGGCAGCAGGGCAAC |
| | | GTGTTCAGCTGCAGCGTGATGCACGAGGCCCTGCAC |
| | | AACCACTACACCCAGAAGTCCCTGAGCCTGAGCCCC |
| | | GGCAAG |
| SEQ ID NO: 142 | E152C/S375C | QIQLVQSGAEVKKPGSSVKVSCKVSGYTFTDHTLHWM |
| | CysMab | RQAPGQGLEWMGYIYPRSGSTKYNENFRGRVTITADTS |
| | Mutated Heavy | SSTAYMELSSLRSEDTAVYYCVRRLLFLPLDYWGQGTL |
| | Chain | VTVSSASTKGPSVFPLAPSSKSTSGGTAALGCLVKDYFP |
| | | CPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPCDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 143 | K360C | QIQLVQSGAEVKKPGSSVKVSCKVSGYTFTDHTLHWM |
| | CysMab | RQAPGQGLEWMGYIYPRSGSTKYNENFRGRVTITADTS |
| | Mutated Heavy | SSTAYMELSSLRSEDTAVYYCVRRLLFLPLDYWGQGTL |
| | Chain | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTCNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 91 | LCDR1 | RSSQSLLSSGNQKSYLT |
| | (Kabat) | |
| SEQ ID NO: 92 | LCDR2 | WASTRES |
| | (Kabat) | |
| SEQ ID NO: 93 | LCDR3 | QNDYSYPFT |
| | (Kabat) | |
| SEQ ID NO: 94 | LCDR1 | SQSLLSSGNQKSY |
| | (Chothia) | |
| SEQ ID NO: 95 | LCDR2 | WAS |
| | (Chothia) | |
| SEQ ID NO: 96 | LCDR3 | DYSYPF |
| | (Chothia) | |
| SEQ ID NO: 97 | VL | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIK |
| SEQ ID NO: 98 | DNA VL | GAGATCGTGATGACTCAGTCACCCGCTACCCTGAGC |
| | | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAG |
| SEQ ID NO: 99 | Light Chain | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| SEQ ID NO: 100 | DNA Light | GAGATCGTGATGACTCAGTCACCCGCTACCCTGAGC |
| | Chain | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAGCGTACGGTGGCCGCTCCCAGCGTGTT |
| | | CATCTTCCCCCCCAGCGACGAGCAGCTGAAGAGCGG |
| | | CACCGCCAGCGTGGTGTGCCTGCTGAACAACTTCTAC |
| | | CCCCGGGAGGCCAAGGTGCAGTGGAAGGTGGACAA |
| | | CGCCCTGCAGAGCGGCAACAGCCAGGAGAGCGTCAC |
| | | CGAGCAGGACAGCAAGGACTCCACCTACAGCCTGAG |
| | | CAGCACCCTGACCCTGAGCAAGGCCGACTACGAGAA |
| | | GCATAAGGTGTACGCCTGCGAGGTGACCCACCAGGG |
| | | CCTGTCCAGCCCCGTGACCAAGAGCTTCAACAGGGG |
| | | CGAGTGC |
| SEQ ID NO: 144 | K107C | EIVMTQSPATLSLSPGERATLSCRSSQSLLSSGNQKSYL |
| | CysMab | TWYQQKPGQAPRLLIYWASTRESGIPARFSGSGSGTDF |
| | Mutated Light | TLTISSLQPEDFAVYYCQNDYSYPFTFGQGTKLEICRTV |
| | Chain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

NEG0067

| | | |
|---|---|---|
| SEQ ID NO: 101 | HCDR1 | DHTLH |
| | (Kabat) | |
| SEQ ID NO: 102 | HCDR2 | YIYPRSGSTKYNENFKG |
| | (Kabat) | |
| SEQ ID NO: 103 | HCDR3 | RLLFLPLDY |
| | (Kabat) | |
| SEQ ID NO: 104 | HCDR1 | GYTFTDH |
| | (Chothia) | |
| SEQ ID NO: 105 | HCDR2 | YPRSGS |
| | (Chothia) | |
| SEQ ID NO: 106 | HCDR3 | RLLFLPLDY |
| | (Chothia) | |
| SEQ ID NO: 107 | VH | QIQLVQSGAEVKKPGSSVKVSCKVSGYTFTDHTLHWM |
| | | RQAPGQGLEWMGYIYPRSGSTKYNENFKGRVTITADTS |
| | | SSTAYMELSSLRSEDTAVYYCVRRLLFLPLDYWGQGTL |
| | | VTVSS |
| SEQ ID NO: 108 | DNA VH | CAGATTCAGCTGGTGCAGTCAGGCGCCGAAGTGAAG |
| | | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAAGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGTC |
| | | AGACGGCTGCTGTTCCTGCCCCTGGACTACTGGGGTC |
| | | AGGGCACCCTGGTCACCGTGTCTAGC |
| SEQ ID NO: 109 | Heavy Chain | QIQLVQSGAEVKKPGSSVKVSCKVSGYTFTDHTLHWM |
| | | RQAPGQGLEWMGYIYPRSGSTKYNENFKGRVTITADTS |
| | | SSTAYMELSSLRSEDTAVYYCVRRLLFLPLDYWGQGTL |
| | | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 110 | DNA Heavy | CAGATTCAGCTGGTGCAGTCAGGCGCCGAAGTGAAG |
| | Chain | AAACCCGGCTCTAGCGTGAAAGTCAGCTGTAAAGTC |
| | | TCAGGCTACACCTTCACCGATCACACCCTGCACTGGA |
| | | TGAGACAGGCCCCAGGTCAGGGCCTGGAGTGGATGG |
| | | GCTATATCTACCCTAGATCAGGCTCTACTAAGTATAA |
| | | CGAGAACTTTAAGGGTAGAGTGACTATCACCGCCGA |
| | | CACTAGCTCTAGCACCGCCTATATGGAACTGTCTAGC |
| | | CTGAGATCAGAGGACACCGCCGTCTACTACTGCGTC |
| | | AGACGGCTGCTGTTCCTGCCCCTGGACTACTGGGGTC |
| | | AGGGCACCCTGGTCACCGTGTCTAGCGCTAGCACTA |
| | | AGGGCCCAAGTGTGTTTCCCCTGGCCCCCAGCAGCA |
| | | AGTCTACTTCCGGCGGAACTGCTGCCCTGGGTTGCCT |
| | | GGTGAAGGACTACTTCCCCGAGCCCGTGACAGTGTC |
| | | CTGGAACTCTGGGGCTCTGACTTCCGGCGTGCACACC |
| | | TTCCCCGCCGTGCTGCAGAGCAGCGGCCTGTACAGC |
| | | CTGAGCAGCGTGGTGACAGTGCCCTCCAGCTCTCTG |
| | | GGAACCCAGACCTATATCTGCAACGTGAACCACAAG |
| | | CCCAGCAACACCAAGGTGGACAAGAGAGTGGAGCC |
| | | CAAGAGCTGCGACAAGACCCACACCTGCCCCCCCTG |
| | | CCCAGCTCCAGAACTGCTGGGAGGGCCTTCCGTGTTC |
| | | CTGTTCCCCCCCAAGCCCAAGGACACCCTGATGATC |
| | | AGCAGGACCCCCGAGGTGACCTGCGTGGTGGTGGAC |
| | | GTGTCCCACGAGGACCCAGAGGTGAAGTTCAACTGG |
| | | TACGTGGACGGCGTGGAGGTGCACAACGCCAAGACC |
| | | AAGCCCAGAGAGGAGCAGTACAACAGCACCTACAG |
| | | GGTGGTGTCCGTGCTGACCGTGCTGCACCAGGACTG |
| | | GCTGAACGGCAAAGAATACAAGTGCAAAGTCTCCAA |
| | | CAAGGCCCTGCCAGCCCCAATCGAAAAGACAATCAG |
| | | CAAGGCCAAGGGCCAGCCACGGGAGCCCCAGGTGTA |
| | | CACCCTGCCCCCCAGCCGGGAGGAGATGACCAAGAA |
| | | CCAGGTGTCCCTGACCTGTCTGGTGAAGGGCTTCTAC |
| | | CCCAGCGATATCGCCGTGGAGTGGGAGAGCAACGGC |
| | | CAGCCCGAGAACAACTACAAGACCACCCCCCCAGTG |
| | | CTGGACAGCGACGGCAGCTTCTTCCTGTACAGCAAG |
| | | CTGACCGTGGACAAGTCCAGGTGGCAGCAGGGCAAC |
| | | GTGTTCAGCTGCAGCGTGATGCACGAGGCCCTGCAC |
| | | AACCACTACACCCAGAAGTCCCTGAGCCTGAGCCCC |
| | | GGCAAG |
| SEQ ID NO: 145 | E152C/S375C | QIQLVQSGAEVKKPGSSVKVSCKVSGYTFTDHTLHWM |
| | CysMab | RQAPGQGLEWMGYIYPRSGSTKYNENFKGRVTITADTS |
| | Mutated Heavy | SSTAYMELSSLRSEDTAVYYCVRRLLFLPLDYWGQGTL |
| | Chain | VTVSSASTKGPSVFPLAPSSKSTSGGTAALGCLVKDYFP |
| | | CPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTKNQVSLTCLVKGFYPCDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 146 | K360C | QIQLVQSGAEVKKPGSSVKVSCKVSGYTFTDHTLHWM |
| | CysMab | RQAPGQGLEWMGYIYPRSGSTKYNENFKGRVTITADTS |
| | Mutated Heavy | SSTAYMELSSLRSEDTAVYYCVRRLLFLPLDYWGQGTL |
| | Chain | VTVSSASTKGPSVFPLAPSSKSTSGGTAALGCLVKDYFP |
| | | EPVTVSWNSGALTSGVHTFPAVLQSSGLYSLSSVVTVP |
| | | SSSLGTQTYICNVNHKPSNTKVDKRVEPKSCDKTHTCP |
| | | PCPAPELLGGPSVFLFPPKPKDTLMISRTPEVTCVVVDV |
| | | SHEDPEVKFNWYVDGVEVHNAKTKPREEQYNSTYRV |
| | | VSVLTVLHQDWLNGKEYKCKVSNKALPAPIEKTISKAK |
| | | GQPREPQVYTLPPSREEMTCNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| SEQ ID NO: 111 | LCDR1 | RSSQSLLSSGNQKSYLT |
| | (Kabat) | |
| SEQ ID NO: 112 | LCDR2 | WASTRES |
| | (Kabat) | |
| SEQ ID NO: 113 | LCDR3 | QNDYSYPFT |
| | (Kabat) | |
| SEQ ID NO: 114 | LCDR1 | SQSLLSSGNQKSY |
| | (Chothia) | |
| SEQ ID NO: 115 | LCDR2 | WAS |
| | (Chothia) | |
| SEQ ID NO: 116 | LCDR3 | DYSYPF |
| | (Chothia) | |
| SEQ ID NO: 117 | VL | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIK |
| SEQ ID NO: 118 | DNA VL | GAGATCGTGATGACTCAGTCACCCGCTACCCTGAGC |
| | | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAG |
| SEQ ID NO: 119 | Light Chain | EIVMTQSPATLSLSPGERATLSCRSSQSLLSSGNQKSYL |
| | | TWYQQKPGQAPRLLIYWASTRESGIPARFSGSGSGTDF |
| | | TLTISSLQPEDFAVYYCQNDYSYPFTFGQGTKLEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| SEQ ID NO: 120 | DNA Light | GAGATCGTGATGACTCAGTCACCCGCTACCCTGAGC |
| | Chain | CTGAGCCCTGGCGAGAGAGCTACACTGAGCTGTAGA |
| | | TCTAGTCAGTCACTGCTGTCTAGCGGTAATCAGAAGT |
| | | CCTACCTGACCTGGTATCAGCAGAAGCCCGGTCAGG |
| | | CCCCTAGACTGCTGATCTACTGGGCCTCTACTAGAGA |
| | | GTCAGGGATCCCCGCTAGGTTTAGCGGTAGCGGTAG |
| | | TGGCACCGACTTCACCCTGACTATCTCTAGCCTGCAG |
| | | CCCGAGGACTTCGCCGTCTACTACTGTCAGAACGACT |
| | | ATAGCTACCCCTTCACCTTCGGTCAGGGCACTAAGCT |
| | | GGAGATTAAGCGTACGGTGGCCGCTCCCAGCGTGTT |
| | | CATCTTCCCCCCCAGCGACGAGCAGCTGAAGAGCGG |
| | | CACCGCCAGCGTGGTGTGCCTGCTGAACAACTTCTAC |
| | | CCCCGGGAGGCCAAGGTGCAGTGGAAGGTGGACAA |
| | | CGCCCTGCAGAGCGGCAACAGCCAGGAGAGCGTCAC |
| | | CGAGCAGGACAGCAAGGACTCCACCTACAGCCTGAG |
| | | CAGCACCCTGACCCTGAGCAAGGCCGACTACGAGAA |
| | | GCATAAGGTGTACGCCTGCGAGGTGACCCACCAGGG |
| | | CCTGTCCAGCCCCGTGACCAAGAGCTTCAACAGGGG |
| | | CGAGTGC |
| SEQ ID NO: 147 | K107C | EIVMTQSPATLSLSPGERATLSCRSSQSLLSSGNQKSYL |
| | CysMab | TWYQQKPGQAPRLLIYWASTRESGIPARFSGSGSGTDF |
| | Mutated Light | TLTISSLQPEDFAVYYCQNDYSYPFTFGQGTKLEICRTV |
| | Chain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |

Cys Mab Mutations to Antibody Constant Regions

| | | |
|---|---|---|
| SEQ ID NO: 148 | E152C/S375C | SASTKGPSVFPLAPSSKSTSGGTAALGCLVKDYFPCPVT |
| | Cys Mab | VSWNSGALTSGVHTFPAVLQSSGLYSLSSVVTVPSSSL |
| | Mutations to | GTQTYICNVNHKPSNTKVDKKVEPKSCDKTHTCPPCPA |
| | Wild Type | PELLGGPSVFLFPPKPKDTLMISRTPEVTCVVVDVSHED |
| | Heavy Chain | PEVKFNWYVDGVEVHNAKTKPREEQYNSTYRVVSVLT |
| | Constant | VLHQDWLNGKEYKCKVSNKALPAPIEKTISKAKGQPR |
| | Region | EPQVYTLPPSREEMTKNQVSLTCLVKGFYPCDIAVEWE |
| | | SNGQPENNYKTTPPVLDSDGSFFLYSKLTVDKSRWQQ |
| | | GNVFSCSVMHEALHNHYTQKSLSLSPGK |
| SEQ ID NO: 149 | K360C Cys | SASTKGPSVFPLAPSSKSTSGGTAALGCLVKDYFPEPVT |
| | Mab Mutations | VSWNSGALTSGVHTFPAVLQSSGLYSLSSVVTVPSSSL |
| | to Wild Type | GTQTYICNVNHKPSNTKVDKKVEPKSCDKTHTCPPCPA |
| | Heavy Chain | PELLGGPSVFLFPPKPKDTLMISRTPEVTCVVVDVSHED |
| | Constant | PEVKFNWYVDGVEVHNAKTKPREEQYNSTYRVVSVLT |
| | Region | VLHQDWLNGKEYKCKVSNKALPAPIEKTISKAKGQPR |
| | | EPQVYTLPPSREEMTCNQVSLTCLVKGFYPSDIAVEWE |
| | | SNGQPENNYKTTPPVLDSDGSFFLYSKLTVDKSRWQQ |
| | | GNVFSCSVMHEALHNHYTQKSLSLSPGK |
| SEQ ID NO: 150 | K107C Cys | CRTVAAPSVFIFPPSDEQLKSGTASVVCLLNNFYPREAK |
| | Mab Mutations | VQWKVDNALQSGNSQESVTEQDSKDSTYSLSSTLTLSK |
| | to Wild Type | ADYEKHKVYACEVTHQGLSSPVTKSFNRGEC |
| | Light Chain | |
| | Constant | |
| | Region | |

Other antibodies of the invention include those where the amino acids or nucleic acids encoding the amino acids have been mutated, yet have at least 60, 70, 80, 90 or 95 percent identity to the sequences described in Table 2. In some embodiments, 1, 2, 3, 4 or 5 amino acids have been mutated in the variable regions when compared with the variable regions depicted in the sequence described in Table 2, while retaining substantially the same therapeutic activity as the antibodies listed in Table 2.

In some embodiments antibodies or antibody fragments (e.g., antigen binding fragment) useful in immunoconjugates of the invention include modified or engineered antibodies, such as an antibody modified to introduce one or more cysteine residues as sites for conjugation to a drug moiety (Junutula J R, et al.: Nat Biotechnol 2008, 26:925-932). In one embodiment, the invention provides a modified antibody or antibody fragment thereof comprising a substitution of one or more amino acids with cysteine at the positions described herein. Sites for cysteine substitution are in the constant regions of the antibody and are thus applicable to a variety of antibodies, and the sites are selected to provide stable and homogeneous conjugates. A modified antibody or fragment can have two or more cysteine substitutions, and these substitutions can be used in combination with other antibody modification and conjugation methods as described herein.Methods for inserting cysteine at specific locations of an antibody are known in the art, see, e.g., Lyons et al, (1990) Protein Eng., 3:703-708, WO 2011/005481, WO2014/124316. In certain embodiments a modified antibody or antibody fragment comprises a substitution of one or more amino acids with cysteine on its constant region selected from positions 117, 119, 121, 124, 139, 152, 153, 155, 157, 164, 169, 171, 174, 189, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody or antibody fragment, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain.Exemplary embodiments of these positions are illustrated in the constant region sequences disclosed in SEQ ID NOs: 148, 149, and 150. Specific embodiments of these positions are disclosed for the anti-P-cadherin antibody sequences in SEQ ID NOs: 130, 131, 132, 133, 134, 135, 136, 137, 138, 139, 140, 141, 142, 143, 144, 145, 146, and 147.

Since each of these antibodies can bind to P-cadherin, the VH, VL, full length light chain, and full length heavy chain sequences (amino acid sequences and the nucleotide sequences encoding the amino acid sequences) can be “mixed and matched” to create other P-cadherin-binding antibodies of the invention. Such “mixed and matched” P-cadherin-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence. Accordingly, in one aspect, the invention provides an isolated monoclonal antibody or antigen binding region thereof having: a heavy chain variable region comprising an amino acid sequence selected from the group consisting of SEQ ID NOs: 7, 27, 47, 67, 87 and 107; and a light chain variable region comprising an amino acid sequence selected from the group consisting of SEQ ID NOs: 17, 37, 57, 77, 97, and 117; wherein the antibody specifically binds to P-cadherin.

In another aspect, the invention provides (i) an isolated monoclonal antibody having: a full length heavy chain comprising an amino acid sequence that has been optimized for expression in the cell of a mammalian expression system selected from the group consisting of SEQ ID NOs: 9, 29, 49, 69, 89, and 109; and a full length light chain comprising an amino acid sequence that has been optimized for expression in the cell of a mammalian selected from the group consisting of SEQ ID NOs: 19, 39, 59, 79, 99, and 119; or (ii) a functional protein comprising an antigen binding portion thereof.

In another aspect, the present invention provides P-cadherin-binding antibodies that comprise the heavy chain and light chain CDR1s, CDR2s and CDR3s as described in Table 2, or combinations thereof. The amino acid sequences of the VH CDR1s of the antibodies are shown in SEQ ID NOs: 1, 21, 41, 61, 81, and 101. The amino acid sequences of the VH CDR2s of the antibodies and are shown in SEQ ID NOs: 2, 22, 42, 62, 82, and 102. The amino acid sequences of the VH CDR3s of the antibodies are shown in SEQ ID NOs: 3, 23, 43, 63, 83, and 103. The amino acid sequences of the VL CDR1s of the antibodies are shown in SEQ ID NOs: 11, 31, 51, 71, 91, and 111. The amino acid sequences of the VL CDR2s of the antibodies are shown in SEQ ID NOs 12, 32, 52, 72, 92, and 112. The amino acid sequences of the VL CDR3s of the antibodies are shown in SEQ ID NOs: 13, 33, 53, 73, 93, and 113.

Given that each of these antibodies can bind to P-cadherin and that antigen-binding specificity is provided primarily by the CDR1, 2 and 3 regions, the VH CDR1, CDR2 and CDR3 sequences and VL CDR1, CDR2 and CDR3 sequences can be “mixed and matched” (i.e., CDRs from different antibodies can be mixed and matched. Such “mixed and matched” P-cadherin-binding antibodies can be tested using the binding assays known in the art and those described in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present invention.

Accordingly, the present invention provides an isolated monoclonal antibody or antigen binding region thereof comprising a heavy chain CDR1 comprising an amino acid sequence selected from the group consisting of SEQ ID NOs: 1, 21, 41, 61, 81, and 101; a heavy chain CDR2 comprising an amino acid sequence selected from the group consisting of SEQ ID NOs: 2, 22, 42, 62, 82, and 102; a heavy chain CDR3 comprising an amino acid sequence selected from the group consisting of SEQ ID NOs: 3, 23, 43, 63, 83, and 103; a light chain CDR1 comprising an amino acid sequence selected from the group consisting of SEQ ID NOs: 11, 31, 51, 71, 91, and 111; a light chain CDR2 comprising an amino acid sequence selected from the group consisting of SEQ ID NOs: 12, 32, 52, 72, 92, and 112; and a light chain CDR3 comprising an amino acid sequence selected from the group consisting of SEQ ID NOs: 13, 33, 53, 73, 93, and 113; wherein the antibody specifically binds P-cadherin.

In a specific embodiment, an antibody or antibody fragment (e.g., antigen binding fragments) that specifically binds to P-cadherin comprises a heavy chain CDR1 of SEQ ID NO:1, a heavy chain CDR2 of SEQ ID NO: 2; a heavy chain CDR3 of SEQ ID NO:3; a light chain CDR1 of SEQ ID NO:11; a light chain CDR2 of SEQ ID NO: 12; and a light chain CDR3 of SEQ ID NO: 13.

In another specific embodiment, an antibody or antibody fragment (e.g., antigen binding fragments) that specifically binds to P-cadherin comprising a heavy chain CDR1 of SEQ ID NO:21, a heavy chain CDR2 of SEQ ID NO: 22; a heavy chain CDR3 of SEQ ID NO:23; a light chain CDR1 of SEQ ID NO:31; a light chain CDR2 of SEQ ID NO: 32; and a light chain CDR3 of SEQ ID NO: 33.

In a yet another embodiment, an antibody or antibody fragment (e.g., antigen binding fragments) that specifically binds to P-cadherin comprising a heavy chain CDR1 of SEQ ID NO:41, a heavy chain CDR2 of SEQ ID NO: 42; a heavy chain CDR3 of SEQ ID NO:43; a light chain CDR1 of SEQ ID NO:51; a light chain CDR2 of SEQ ID NO: 52; and a light chain CDR3 of SEQ ID NO: 53.

In a further embodiment, an antibody or antibody fragment (e.g., antigen binding fragments) that specifically binds to P-cadherin comprising a heavy chain CDR1 of SEQ ID NO:61, a heavy chain CDR2 of SEQ ID NO: 62; a heavy chain CDR3 of SEQ ID NO:63; a light chain CDR1 of SEQ ID NO:71; a light chain CDR2 of SEQ ID NO: 72; and a light chain CDR3 of SEQ ID NO: 73.

In another specific embodiment, an antibody or antibody fragment (e.g., antigen binding fragments) that specifically binds to P-cadherin comprising a heavy chain CDR1 of SEQ ID NO:81, a heavy chain CDR2 of SEQ ID NO: 82; a heavy chain CDR3 of SEQ ID NO:83; a light chain CDR1 of SEQ ID NO:91; a light chain CDR2 of SEQ ID NO: 92; and a light chain CDR3 of SEQ ID NO: 93.

In a further specific embodiment, an antibody or antibody fragment (e.g., antigen binding fragments) that specifically binds to P-cadherin comprising a heavy chain CDR1 of SEQ ID NO:101, a heavy chain CDR2 of SEQ ID NO: 102; a heavy chain CDR3 of SEQ ID NO:103; a light chain CDR1 of SEQ ID NO:111; a light chain CDR2 of SEQ ID NO: 112; and a light chain CDR3 of SEQ ID NO: 113.

In certain embodiments, an antibody that specifically binds to P-cadherin is an antibody or antibody fragment (e.g., antigen binding fragment) that is described in Table 2.

1. Identification of Epitopes and Antibodies that Bind to the Same Epitope

In one embodiment, the present invention provides antibodies or antibody fragments (e.g., antigen binding fragments) that specifically bind to an epitope on human P-cadherin comprising one or more residues selected from the amino acids at positions 124, 125, 151, 153, 154, 155, 156, 159, 160, 161, 162, 163, 168, 170, 171, and 172 of SEQ ID NO:126. In some embodiments, the present invention provides antibodies or antibody fragments (e.g., antigen binding fragments) comprising a heavy chain that binds to human P-cadherin at one or more amino acid residues selected from positions 124, 151, 153-156, and 172 of SEQ ID NO:126. In other embodiments, the present invention provides antibodies or antibody fragments (e.g., antigen binding fragments) comprising a light chain that binds to human P-cadherin at one or more amino acid residues selected from positions 124, 125, 155, 156, 159-163, 168, 170, and 171 of SEQ ID NO:126. In some embodiments, the antibodies or antibody fragments comprise a heavy chain binding paratop for human P-cadherin protein comprising one or more amino acid residues selected from positions 52, 54, 56, 60, 65, 105, or 107 of SEQ ID NO:128. In other embodiments, the antibodies or antibody fragments comprise a light chain binding paratope for human P-cadherin protein comprising one or more amino acid residues selected from positions 1, 2, 27, 28, 30, 68, 92, 93, or 94 of SEQ ID NO:129

The present invention also provides antibodies and antibody fragments (e.g., antigen binding fragments) that specifically bind to the same epitope as the anti-P-cadherin antibodies described in Table 2, or cross compete with the antibodies described in Table 2. Additional antibodies and antibody fragments (e.g., antigen binding fragments) can therefore be identified based on their ability to cross-compete (e.g., to competitively inhibit the binding of, in a statistically significant manner) with other antibodies of the invention in P-cadherin binding assays, for example, via BIACORE or assays known to persons skilled in the art for measuring binding. The ability of a test antibody to inhibit the binding of antibodies and antibody fragments (e.g., antigen binding fragments) of the present invention to a P-cadherin (e.g., human P-cadherin) demonstrates that the test antibody can compete with that antibody or antibody fragment (e.g., antigen binding fragments) for binding to P-cadherin; such an antibody may, according to non-limiting theory, bind to the same or a related (e.g., a structurally similar or spatially proximal or overlapping) epitope on the P-cadherin protein as the antibody or antibody fragment (e.g., antigen binding fragments) with which it competes. In certain embodiments, the antibodies that bind to the same epitope on P-cadherin as the antibodies or antibody fragments (e.g., antigen binding fragments) described in Table 2 are human or humanized monoclonal antibodies. Such human or humanized monoclonal antibodies can be prepared and isolated as described herein.

### 2. Further Alteration of the Framework of Fc Region

The immunoconjugates of the invention may comprise modified antibodies or antigen binding fragments thereof that further comprise modifications to framework residues within VH and/or VL, e.g. to improve the properties of the antibody. In some embodiments, the framework modifications are made to decrease the immunogenicity of the antibody. For example, one approach is to “back-mutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis. Such “back-mutated” antibodies are also intended to be encompassed by the invention.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 20030153043 by Carr et al.

In addition or in the alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in, e.g., U.S. Pat. No. 6,194,551 by Idusogie et al.

In another embodiment, one or more amino acid residues are altered to thereby alter the ability of the antibody to fix complement. This approach is described in, e.g., the PCT Publication WO 94/29351 by Bodmer et al. In a specific embodiment, one or more amino acids of an antibody or antigen binding fragment thereof of the present invention are replaced by one or more allotypic amino acid residues. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the PCT Publication WO 00/42072 by Presta. Moreover, the binding sites on human IgG1 for FcγR1, FcγRII, FcγRIII and FcRn have been mapped and variants with improved binding have been described (see Shields et al., J. Biol. Chem. 276:6591-6604, 2001).

In still another embodiment, the glycosylation of an antibody is modified. For example, an aglycosylated antibody can be made (i.e., the antibody lacks glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in, e.g., U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

### 3. Production of the P-Cadherin Antibodies

Anti-P-cadherin antibodies and antibody fragments (e.g., antigen binding fragments) thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The invention further provides polynucleotides encoding the antibodies described herein, e.g., polynucleotides encoding heavy or light chain variable regions or segments comprising the complementarity determining regions as described herein. In some embodiments, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 8, 28, 48, 68, 88, 108, and 151. In some embodiments, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs:18, 38, 58, 78, 98, 118, and 153.

In some embodiments, the polynucleotide encoding the heavy chain has at least 85%, 89%, 90%, 91%, 92%, 93%, 94%, 95%, 96%, 97%, 98%, 99%, or 100% nucleic acid sequence identity with a polynucleotide of SEQ ID NO: 10, 30, 50, 70, 90, 110, or 152. In some embodiments, the polynucleotide encoding the light chain has at least 85%, 89%, 90%, 91%, 92%, 93%, 94%, 95%, 96%, 97%, 98%, 99%, or 100% nucleic acid sequence identity with a polynucleotide of SEQ ID NO: 20, 40, 60, 80, 100, 120, or 154.

The polynucleotides of the invention can encode only the variable region sequence of an anti-P-cadherin antibody. They can also encode both a variable region and a constant region of the antibody. Some of the polynucleotide sequences encode a polypeptide that comprises variable regions of both the heavy chain and the light chain of one of the exemplified mouse anti-P-cadherin antibody. Some other polynucleotides encode two polypeptide segments that respectively are substantially identical to the variable regions of the heavy chain and the light chain of one of the mouse antibodies.

The polynucleotide sequences can be produced by de novo solid-phase DNA synthesis or by PCR mutagenesis of an existing sequence (e.g., sequences as described in the Examples below) encoding an anti-P-cadherin antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the anti-P-cadherin antibodies described above. Various expression vectors can be employed to express the polynucleotides encoding the anti-P-cadherin antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). For example, nonviral vectors useful for expression of the anti-P-cadherin polynucleotides and polypeptides in mammalian (e.g., human) cells include pThioHis A, B & C, pcDNA™ 3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-P-cadherin antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an anti-P-cadherin antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-P-cadherin antibody sequences. More often, the inserted anti-P-cadherin antibody sequences are linked to a signal sequences before inclusion in the vector. Vectors to be used to receive sequences encoding anti-P-cadherin antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the anti-P-cadherin antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-P-cadherin polypeptides of the invention. Insect cells in combination with baculovirus vectors can also be used.

In some preferred embodiments, mammalian host cells are used to express and produce the anti-P-cadherin polypeptides of the present invention. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-P-cadherin antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### Therapeutic and Diagnostic Uses

The antibodies, antibody fragments (e.g., antigen binding fragments), and antibody drug conjugates of the invention are useful in a variety of applications including, but not limited to, treatment of cancer, such as solid cancers. In certain embodiments, the antibodies, antibody fragments (e.g., antigen binding fragments), and antibody drug conjugates of the invention are useful for inhibiting tumor growth, inducing differentiation, reducing tumor volume, and/or reducing the tumorigenicity of a tumor. The methods of use can be in vitro, ex vivo, or in vivo methods.

In one aspect, the antibodies, antibody fragments (e.g., antigen binding fragments), and antibody drug conjugates of the invention are useful for detecting the presence of P-cadherin in a biological sample. The term “detecting” as used herein encompasses quantitative or qualitative detection. In certain embodiments, a biological sample comprises a cell or tissue. In certain embodiments, such tissues include normal and/or cancerous tissues that express P-cadherin at higher levels relative to other tissues.

In one aspect, the invention provides a method of detecting the presence of P-cadherin in a biological sample. In certain embodiments, the method comprises contacting the biological sample with an anti-P-cadherin antibody under conditions permissive for binding of the antibody to the antigen, and detecting whether a complex is formed between the antibody and the antigen.

In one aspect, the invention provides a method of diagnosing a disorder associated with increased expression of P-cadherin. In certain embodiments, the method comprises contacting a test cell with an anti-P-cadherin antibody; determining the level of expression (either quantitatively or qualitatively) of P-cadherin on the test cell by detecting binding of the anti-P-cadherin antibody to the P-cadherin antigen; and comparing the level of expression of P-cadherin in the test cell with the level of expression of P-cadherin on a control cell (e.g., a normal cell of the same tissue origin as the test cell or a cell that expresses P-cadherin at levels comparable to such a normal cell), wherein a higher level of expression of P-cadherin on the test cell as compared to the control cell indicates the presence of a disorder associated with increased expression of P-cadherin . In certain embodiments, the test cell is obtained from an individual suspected of having a disorder associated with increased expression of P-cadherin. In certain embodiments, the disorder is a cell proliferative disorder, such as a cancer or a tumor. In certain embodiments, the method comprises measuring the copy number of the P-cadherin gene in a test cell. In certain embodiments, the method comprises detecting a PAX-FOXO translocation mutation. Copy number of a gene and/or translocation mutations can be detected using standard methods known in the art, for example, PCR, RTPCR, etc.

In certain embodiments, a method of diagnosis or detection, such as those described above, comprises detecting binding of an anti-P-cadherin antibody to P-cadherin expressed on the surface of a cell or in a membrane preparation obtained from a cell expressing P-cadherin on its surface. An exemplary assay for detecting binding of an anti-P-cadherin antibody to P-cadherin expressed on the surface of a cell is a “FACS” assay.

Certain other methods can be used to detect binding of anti-P-cadherin antibodies to P-cadherin. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain embodiments, anti-P-cadherin antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In certain embodiments, anti-P-cadherin antibodies are immobilized on an insoluble matrix. Immobilization entails separating the anti-P-cadherin antibody from any P-cadherin protein that remains free in solution. This conventionally is accomplished by either insolubilizing the anti-P-cadherin antibody before the assay procedure, as by adsorption to a water-insoluble matrix or surface (Bennich et al, U.S. Pat. No. 3,720,760), or by covalent coupling (for example, using glutaraldehyde cross-linking), or by insolubilizing the anti-P-cadherin antibody after formation of a complex between the anti-P-cadherin antibody and P-cadherin protein, e.g., by immunoprecipitation.

Any of the above embodiments of diagnosis or detection can be carried out using an immunoconjugate of the invention in place of or in addition to an anti-P-cadherin antibody.

In one embodiment, the invention provides a method of treating, or preventing a disease comprising administering the antibodies, antibody fragments (e.g., antigen binding fragments), or antibody drug conjugates of the invention to a patient. The invention also provides use of the antibodies, antibody fragments (e.g. antigen binding fragments, or antibody drug conjugates of the invention to treat or prevent disease in a patient. In some embodiments, the invention provides antibodies, antibody fragments (e.g. antigen binding fragments, or antibody drug conjugates of the invention for use in the treatment or prevention of disease in a patient. In further embodiments, the invention provides use of the antibodies, antibody fragments (e.g. antigen binding fragments, or antibody drug conjugates of the invention in the manufacture of a medicament for treatment or prevention of disease in a patient.

In certain embodiments, the disease treated with the antibodies, antibody fragments (e.g., antigen binding fragments), and antibody drug conjugates of the invention is a cancer. In certain embodiments, the cancer is characterized by P-cadherin expressing cells to which the antibodies, antibody fragments (e.g., antigen binding fragments), and antibody drug conjugates of the invention binds. In certain embodiments, the cancer is characterized by an increase in expression of P-cadherin relative to a healthy patient. In some embodiments, the expression of P-cadherin may be measured by an increase in P-cadherin RNA. In other embodiments, the cancer is characterized by an increase in DNA copy number of P-cadherin. Other methods of measuring or determining levels of p-Cadherin expression are known to persons skilled in the art. Examples of diseases which can be treated and/or prevented include, but are not limited to, adrenocortical carcinoma, bladder cancer, bone cancer, breast cancer, central nervous system atypical teratoid/rhabdoid tumors, colon cancer, colorectal cancer, embryonal tumors, endometrial cancer, esophageal cancer, gastric cancer, head and neck cancer, hepatocellular cancer, Kaposi sarcoma, liver cancer, lung cancer, including small cell lung cancer and non-small cell lung cancer, ovarian cancer, rectal cancer, rhabdomyosarcomasmall intestine cancer, soft tissue sarcoma, squamous cell carcinoma, squamous neck cancer, stomach cancer, uterine cancer, vaginal cancer, and vulvar cancer.

The present invention provides for methods of treating cancer comprising administering a therapeutically effective amount of the antibodies, antibody fragments (e.g., antigen binding fragments), or antibody drug conjugates of the invention. In certain embodiments, the cancer is a solid cancer. In certain embodiments, the subject is a human. In certain embodiments, the cancer is a resistant cancer and/or relapsed cancer. In certain aspects, for example, the resistant cancer is resistant to tyrosine kinase inhibitors, including but not limited to, EGFR inhibitors, Her2 inhibitors, Her3 inhibitors, IGFR inhibitors and Met inhibitors. In certain embodiments the cancer is a Her2 resistant cancer.

In certain embodiments, the invention provides for methods of inhibiting tumor growth comprising administering to a subject a therapeutically effective amount of the antibodies, antibody fragments (e.g., antigen binding fragments), or antibody drug conjugates of the invention. In certain embodiments, the subject is a human. In certain embodiments, the subject has a tumor or has had a tumor removed. In certain embodiments, the tumor is resistant to other tyrosine kinase inhibitors, including but not limited to, EGFR inhibitors, Her2 inhibitors, Her3 inhibitors, IGFR inhibitors and Met inhibitors.

In certain embodiments, the tumor expresses the P-cadherin to which the anti-P-cadherin antibody binds. In certain embodiments, the tumor overexpresses the human P-cadherin. In certain embodiments, the tumor has an increase copy number of the P-cadherin gene.

The present invention also provides for methods of selecting patients for treatment with antibodies, antibody fragments (e.g., antigen binding fragments), or antibody drug conjugates of the invention comprising administering a therapeutically effective amount of said antibodies, antibody fragments (e.g., antigen binding fragments), or antibody drug conjugates. In certain aspects the method comprises selecting patients with a tyrosine kinase inhibitor resistant cancer. In certain aspects it is contemplated that the tyrosine kinase inhibitor resistant cancer is resistant to EGFR inhibitors, Her2 inhibitors, Her3 inhibitors, IGFR inhibitors and/or Met inhibitors. In certain aspects it is contemplated that the resistant cancer is a Her2 resistant cancer. More specifically it is contemplated that the Her2 resistant cancer does not respond to trastuzumab or trastuzumab emtansine. In certain aspects it is contemplated that the cancer is a de novo resistant cancer, and in still other aspects it is contemplated that the cancer is a relapsed cancer, for example a Her2 relapsed cancer. In certain aspects of the invention the methods comprise selecting a patient with a de novo resistant or relapsed cancer and measuring for expression of P-cadherin. It is contemplated that in certain aspects the relapsed cancer or tumor was not initially a P-cadherin expressing cancer or tumor, but becomes a P-cadherin positive cancer that is a tyrosine kinase resistant or relapsed cancer or tumor after treatment with tyrosine kinase inhibitors (for example, trastuzumab or trastuzumab emtansine).

For the treatment of the disease, the appropriate dosage of the antibodies, antibody fragments (e.g., antigen binding fragments), or antibody drug conjugates of the present invention depends on various factors, such as the type of disease to be treated, the severity and course of the disease, the responsiveness of the disease, previous therapy, patient's clinical history, and so on. The antibody or agent can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody, antibody fragment (e.g., antigen binding fragment), or antibody drug conjugates. The treating physician can estimate repetition rates for dosing based on measured residence times and concentrations of the drug in bodily fluids or tissues.

### Combination Therapy

In certain instances, an antibody, antibody fragment (e.g., antigen binding fragment), or antibody drug conjugate of the present invention is combined with other therapeutic agents, such as other anti-cancer agents, anti-allergic agents, anti-nausea agents (or anti-emetics), pain relievers, cytoprotective agents, and combinations thereof.

In one embodiment, an antibody, antibody fragment (e.g., antigen binding fragment), or antibody drug conjugate of the present invention is combined in a pharmaceutical combination formulation, or dosing regimen as combination therapy, with a second compound having anti-cancer properties. The second compound of the pharmaceutical combination formulation or dosing regimen can have complementary activities to the antibody or immunoconjugate of the combination such that they do not adversely affect each other. For example, an antibody, antibody fragment (e.g., antigen binding fragment), or antibody drug conjugate of the present invention can be administered in combination with, but not limited to, a chemotherapeutic agent, a tyrosine kinase inhibitor, a P-cadherin downstream signaling pathway inhibitor, IAP inhibitors, Bcl2 inhibitors, Mcl1 inhibitors, and other P-cadherin inhibitors.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In one aspect, the present invention provides a method of treating cancer by administering to a subject in need thereof an antibody drug conjugate of the present invention in combination with one or more tyrosine kinase inhibitors, including but not limited to, EGFR inhibitors, Her2 inhibitors, Her3 inhibitors, IGFR inhibitors, and Met inhibitors.

For example, tyrosine kinase inhibitors include but are not limited to, Erlotinib hydrochloride (Tarceva®); Linifanib (N-[4-(3-amino-1H-indazol-4-yl)phenyl]-N′-(2-fluoro-5-methylphenyl)urea, also known as ABT 869, available from Genentech); Sunitinib malate (Sutent®); Bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); Dasatinib (Sprycel®); Pazopanib (Votrient®); Sorafenib (Nexavar®); Zactima (ZD6474); and Imatinib or Imatinib mesylate (Gilvec® and Gleevec®).

Epidermal growth factor receptor (EGFR) inhibitors include but are not limited to, Erlotinib hydrochloride (Tarceva®), Gefitinib (Iressa®); N-[4-[(3-Chloro-4-fluorophenyl)amino]-7-[[(3″S″)-tetrahydro-3-furany]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

EGFR antibodies include but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1).

Human Epidermal Growth Factor Receptor 2 (Her2 receptor) (also known as Neu, ErbB-2, CD340, or p185) inhibitors include but are not limited to, Trastuzumab (Herceptin®); Pertuzumab (Omnitarg®); trastuzumab emtansine (Kadcyla®); Neratinib (HKI-272, (2E)-N-[4-[[3-chloro-4-[(pyridin-2-yl)methoxy]phenyl]amino]-3-cyano-7-ethoxyquinolin-6-yl]-4-(dimethylamino)but-2-enamide, and described PCT Publication No. WO 05/028443); Lapatinib or Lapatinib ditosylate (Tykerb®); (3R,4R)-4-amino-1-((4-((3-methoxyphenyl)amino)pyrrolo[2,1-f][1,2,4]triazin-5-yl)methyl)piperidin-3-ol (BMS690514); (2E)-N-[4-[(3-Chloro-4-fluorophenyl)amino]-7-[[(3S)-tetrahydro-3-furanyl]oxy]-6-quinazolinyl]-4-(dimethylamino)-2-butenamide (BIBW-2992, CAS 850140-72-6); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS 599626, CAS 714971-09-2); Canertinib dihydrochloride (PD183805 or CI-1033); and N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8).

Her3 inhibitors include but are not limited to, LJM716, MM-121, AMG-888, RG7116, REGN-1400, AV-203, MP-RM-1, MM-111, and MEHD-7945A.

MET inhibitors include but are not limited to, Cabozantinib (XL184, CAS 849217-68-1); Foretinib (GSK1363089, formerly XL880, CAS 849217-64-7); Tivantinib (ARQ197, CAS 1000873-98-2); 1-(2-Hydroxy-2-methylpropyl)-N-(5-(7-methoxyquinolin-4-yloxy)pyridin-2-yl)-5-methyl-3-oxo-2-phenyl-2,3-dihydro-1H-pyrazole-4-carboxamide (AMG 458); Cryzotinib (Xalkori®, PF-02341066); (3Z)-5-(2,3-Dihydro-1H-indol-1-ylsulfonyl)-3-({3,5-dimethyl-4-[(4-methylpiperazin-1-yl)carbonyl]-1H-pyrrol-2-yl}methylene)-1,3-dihydro-2H-indol-2-one (SU11271); (3Z)-N-(3-Chlorophenyl)-3-({3,5-dimethyl-4-[(4-methylpiperazin-1-yl)carbonyl]-1H-pyrrol-2-yl}methylene)-N-methyl-2-oxoindoline-5-sulfonamide (SU11274); (3Z)-N-(3-Chlorophenyl)-3-{[3,5-dimethyl-4-(3-morpholin-4-ylpropyl)-1H-pyrrol-2-yl]methylene}-N-methyl-2-oxoindoline-5-sulfonamide (SU11606); 6-[Difluoro[6-(1-methyl-1H-pyrazol-4-yl)-1,2,4-triazolo[4,3-b]pyridazin-3-yl]methyl]-quinoline (JNJ38877605, CAS 943540-75-8); 2-[4-[1-(Quinolin-6-ylmethyl)-1H-[1,2,3]triazolo[4,5-b]pyrazin-6-yl]-1H-pyrazol-1-yl]ethanol (PF04217903, CAS 956905-27-4); N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide (MK2461, CAS 917879-39-1); 6-[[6-(1-Methyl-1H-pyrazol-4-yl)-1,2,4-triazolo[4,3-b]pyridazin-3-yl]thio]-quinoline (SGX523, CAS 1022150-57-7); and (3Z)-5-[[(2,6-Dichlorophenyl)methyl]sulfonyl]-3-[[3,5-dimethyl-4-[[(2R)-2-(1-pyrrolidinylmethyl)-1-pyrrolidinyl]carbonyl]-1H-pyrrol-2-yl]methylene]-1,3-dihydro-2H-indol-2-one (PHA665752, CAS 477575-56-7).

IGF1R inhibitors include but are not limited to, BMS-754807, XL-228, OSI-906, GSK0904529A, A-928605, AXL1717, KW-2450, MK0646, AMG479, IMCA12, MEDI-573, and BI836845. See e.g., Yee, JNCI, 104; 975 (2012) for review.

In another aspect, the present invention provides a method of treating cancer by administering to a subject in need thereof an antibody drug conjugate of the present invention in combination with one or more P-cadherin downstream signaling pathway inhibitors, including but not limited to, MEK inhibitors, Braf inhibitors, PI3K/Akt inhibitors, SHP2 inhibitors, and also mTor.

For example, mitogen-activated protein kinase (MEK) inhibitors include but are not limited to, XL-518 (also known as GDC-0973, Cas No. 1029872-29-4, available from ACC Corp.); 2-[(2-Chloro-4-iodophenyl)amino]-N-(cyclopropylmethoxy)-3,4-difluoro-benzamide (also known as CI-1040 or PD184352 and described in PCT Publication No. WO2000035436); N-[(2R)-2,3-Dihydroxypropoxy]-3,4-difluoro-2-[(2-fluoro-4-iodophenyl)amino]-benzamide (also known as PD0325901 and described in PCT Publication No. WO2002006213); 2,3-Bis[amino[(2-aminophenyl)thio]methylene]-butanedinitrile (also known as U0126 and described in U.S. Pat. No. 2,779,780); N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO2007014011); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201 and described in PCT Publication No. WO2003076424); 2′-Amino-3′-methoxyflavone (also known as PD98059 available from Biaffin GmbH & Co., KG, Germany); Vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); Pimasertib (AS-703026, CAS 1204531-26-9); and Trametinib dimethyl sulfoxide (GSK-1120212, CAS 1204531-25-80).

Phosphoinositide 3-kinase (PI3K) inhibitors include but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6).

mTor include but are not limited to, Temsirolimus (Torisel®); Ridaforolimus (formally known as deferolimus, (1R,2R,4S)-4-[(2R)-2[(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1).

In yet another aspect, the present invention provides a method of treating cancer by administering to a subject in need thereof an antibody drug conjugate of the present invention in combination with one or more pro-apoptotics, including but not limited to, IAP inhibitors, Bcl2 inhibitors, MCl1 inhibitors, Trail agents, Chk inhibitors.

For examples, IAP inhibitors include but are not limited to, LCL161, GDC-0917, AEG-35156, AT406, and TL32711. Other examples of IAP inhibitors include but are not limited to those disclosed in WO04/005284, WO 04/007529, WO05/097791, WO 05/069894, WO 05/069888, WO 05/094818, US2006/0014700, US2006/0025347, WO 06/069063, WO 06/010118, WO 06/017295, and WO08/134679, all of which are incorporated herein by reference.

BCL-2 inhibitors include but are not limited to, 4-[4-[[2-(4-Chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide (also known as ABT-263 and described in PCT Publication No. WO 09/155386); Tetrocarcin A; Antimycin; Gossypol ((−)BL-193); Obatoclax; Ethyl-2-amino-6-cyclopentyl-4-(1-cyano-2-ethoxy-2-oxoethyl)-4Hchromone-3-carboxylate (HA14-1); Oblimersen (G3139, Genasense®); Bak BH3 peptide; (−)-Gossypol acetic acid (AT-101); 4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide (ABT-737, CAS 852808-04-9); and Navitoclax (ABT-263, CAS 923564-51-6).

Proapoptotic receptor agonists (PARAs) including DR4 (TRAILR1) and DR5 (TRAILR2), including but are not limited to, Dulanermin (AMG-951, RhApo2L/TRAIL); Mapatumumab (HRS-ETR1, CAS 658052-09-6); Lexatumumab (HGS-ETR2, CAS 845816-02-6); Apomab (Apomab®); Conatumumab (AMG655, CAS 896731-82-1); and Tigatuzumab (CS1008, CAS 946415-34-5, available from Daiichi Sankyo).

Checkpoint Kinase (CHK) inhibitors include but are not limited to, 7-Hydroxystaurosporine (UCN-01); 6-Bromo-3-(1-methyl-1H-pyrazol-4-yl)-5-(3R)-3-piperidinyl-pyrazolo[1,5-a]pyrimidin-7-amine (SCH900776, CAS 891494-63-6); 5-(3-Fluorophenyl)-3-ureidothiophene-2-carboxylic acid N—[(S)-piperidin-3-yl]amide (AZD7762, CAS 860352-01-8); 4-[((3S)-1-Azabicyclo[2.2.2.]oct-3-yl)amino]-3-(1H-benzimidazol-2-yl)-6-chloroquinolin-2(1H)-one (CHIR 124, CAS 405168-58-3); 7-Aminodactinomycin (7-AAD), Isogranulatimide, debromohymenialdisine; N-[5-Bromo-4-methyl-2-[(2S)-2-morpholinylmethoxy]-phenyl]-N′-(5-methyl-2-pyrazinyl)urea (LY2603618, CAS 911222-45-2); Sulforaphane (CAS 4478-93-7, 4-Methylsulfinylbutyl isothiocyanate); 9,10,11,12-Tetrahydro-9,12-epoxy-1H-diindolo[1,2,3-fg:3′,2′,1′-kl]pyrrolo[3,4-i][1,6]benzodiazocine-1,3(2H)-dione (SB-218078, CAS 135897-06-2); and TAT-S216A (YGRKKRRQRRRLYRSPAMPENL), and CBP501 ((d-Bpa)sws(d-Phe-F5)(d-Cha)rrrqrr).

In a further embodiment, the present invention provides a method of treating cancer by administering to a subject in need thereof an antibody drug conjugate of the present invention in combination with one or more immunomodulators(e.g., one or more of: an activator of a costimulatory molecule or an inhibitor of an immune checkpoint molecule).

In certain embodiments, the immunomodulator is an activator of a costimulatory molecule. In one embodiment, the agonist of the costimulatory molecule is chosen from an agonist (e.g., an agonistic antibody or antigen-binding fragment thereof, or a soluble fusion) of OX40, CD2, CD27, CDS, ICAM-1, LFA-1 (CD11a/CD18), ICOS (CD278), 4-1BB (CD137), GITR, CD30, CD40, BAFFR, HVEM, CD7, LIGHT, NKG2C, SLAMF7, NKp80, CD160, B7-H3 or CD83 ligand.

In certain embodiments, the immunomodulator is an inhibitor of an immune checkpoint molecule. In one embodiment, the immunomodulator is an inhibitor of PD-1, PD-L1, PD-L2, CTLA4, TIM3, LAG3, VISTA, BTLA, TIGIT, LAIR1, CD160, 2B4 and/or TGFR beta. In one embodiment, the inhibitor of an immune checkpoint molecule inhibits PD-1, PD-L1, LAG-3, TIM-3 or CTLA4, or any combination thereof. The term “inhibition” or “inhibitor” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40%, 50% or more is included by this term. Thus, inhibition need not be 100%.

Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof (also referred to herein as “an antibody molecule”) that binds to PD-1, PD-L1, PD-L2, CTLA4, TIM3, LAG3, VISTA, BTLA, TIGIT, LAIR1, CD160, 2B4 and/or TGFR beta, or a combination thereof.

In one embodiment, the antibody molecule is a full antibody or fragment thereof (e.g., a Fab, F(ab′)₂, Fv, or a single chain Fv fragment (scFv)). In yet other embodiments, the antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG4 (e.g., human IgG1 or IgG4). In one embodiment, the heavy chain constant region is human IgG1 or human IgG4. In one embodiment, the constant region is altered, e.g., mutated, to modify the properties of the antibody molecule (e.g., to increase or decrease one or more of: Fc receptor binding, antibody glycosylation, the number of cysteine residues, effector cell function, or complement function).

In certain embodiments, the antibody molecule is in the form of a bispecific or multispecific antibody molecule. In one embodiment, the bispecific antibody molecule has a first binding specificity to PD-1 or PD-L1 and a second binding specifity, e.g., a second binding specificity to TIM-3, LAG-3, or PD-L2. In one embodiment, the bispecific antibody molecule binds to PD-1 or PD-L1 and TIM-3. In another embodiment, the bispecific antibody molecule binds to PD-1 or PD-L1 and LAG-3. In another embodiment, the bispecific antibody molecule binds to PD-1 and PD-L1. In yet another embodiment, the bispecific antibody molecule binds to PD-1 and PD-L2. In another embodiment, the bispecific antibody molecule binds to TIM-3 and LAG-3. Any combination of the aforesaid molecules can be made in a multispecific antibody molecule, e.g., a trispecific antibody that includes a first binding specificity to PD-1 or PD-1, and a second and third binding specifities to two or more of: TIM-3, LAG-3, or PD-L2.

In certain embodiments, the immunomodulator is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. The PD-1 or PD-L1 inhibitor can be administered alone, or in combination with other immunomodulators, e.g., in combination with an inhibitor of LAG-3, TIM-3 or CTLA4. In an exemplary embodiment, the inhibitor of PD-1 or PD-L1, e.g., the anti-PD-1 or PD-L1 antibody molecule, is administered in combination with a LAG-3 inhibitor, e.g., an anti-LAG-3 antibody molecule. In another embodiment, the inhibitor of PD-1 or PD-L1, e.g., the anti-PD-1 or PD-L1 antibody molecule, is administered in combination with a TIM-3 inhibitor, e.g., an anti-TIM-3 antibody molecule. In yet other embodiments, the inhibitor of PD-1 or PD-L1, e.g., the anti-PD-1 antibody molecule, is administered in combination with a LAG-3 inhibitor, e.g., an anti-LAG-3 antibody molecule, and a TIM-3 inhibitor, e.g., an anti-TIM-3 antibody molecule. Other combinations of immunomodulators with a PD-1 inhibitor (e.g., one or more of PD-L2, CTLA4, TIM3, LAG3, VISTA, BTLA, TIGIT, LAIR1, CD160, 2B4 and/or TGFR) are also within the present invention. Any of the antibody molecules known in the art or disclosed herein can be used in the aforesaid combinations of inhibitors of checkpoint molecule.

In one embodiment, the PD-1 inhibitor is an anti-PD-1 antibody chosen from Nivolumab, Pembrolizumab or Pidilizumab. In some embodiments, the anti-PD-1 antibody is Nivolumab. Alternative names for Nivolumab include MDX-1106, MDX-1106-04, ONO-4538, or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and PCT Publication No. WO2006/121168.

In other embodiments, the anti-PD-1 antibody is Pembrolizumab. Pembrolizumab (Trade name KEYTRUDA formerly Lambrolizumab,-also known as Merck 3745, MK-3475 or SCH-900475) is a humanized IgG4 monoclonal antibody that binds to PD1. Pembrolizumab is disclosed, e.g., in Hamid, O. et al. (2013) New England Journal of Medicine 369 (2): 134-44, PCT Publication No. WO2009/114335, and U.S. Pat. No. 8,354,509.

In some embodiments, the anti-PD-1 antibody is Pidilizumab. Pidilizumab (CT-011; Cure Tech) is a humanized IgG1k monoclonal antibody that binds to PD1. Pidilizumab and other humanized anti-PD-1 monoclonal antibodies are disclosed in PCT Publication No. WO2009/101611. Other anti-PD1 antibodies are disclosed in U.S. Pat. No. 8,609,089, US Publication No. 2010028330, and/or US Publication No. 20120114649. Other anti-PD1 antibodies include AMP 514 (Amplimmune).

In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224.

In some embodiments, the PD-L1 inhibitor is anti-PD-L1 antibody. In some embodiments, the anti-PD-L1 inhibitor is chosen from YW243.55.570, MPDL3280A, MEDI-4736, or MDX-1105MSB-0010718C (also referred to as A09-246-2) disclosed in, e.g., WO 2013/0179174, and having a sequence disclosed herein (or a sequence substantially identical or similar thereto, e.g., a sequence at least 85%, 90%, 95% identical or higher to the sequence specified).

In one embodiment, the PD-L1 inhibitor is MDX-1105. MDX-1105, also known as BMS-936559, is an anti-PD-L1 antibody described in PCT Publication No. WO2007/005874.

In one embodiment, the PD-L1 inhibitor is YW243.55.570. The YW243.55.570 antibody is an anti-PD-L1 described in PCT Publication No. WO 2010/077634 (heavy and light chain variable region sequences shown in SEQ ID Nos. 20 and 21, respectively).

In one embodiment, the PD-L1 inhibitor is MDPL3280A (Genentech/Roche). MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906.

In other embodiments, the PD-L2 inhibitor is AMP-224. AMP-224 is a PD-L2 Fc fusion soluble receptor that blocks the interaction between PD1 and B7-H1 (B7-DCIg; Amplimmune; e.g., disclosed in PCT Publication Nos. WO2010/027827 and WO2011/066342).

In one embodiment, the LAG-3 inhibitor is an anti-LAG-3 antibody molecule. In one embodiment, the LAG-3 inhibitor is BMS-986016.

### Pharmaceutical Compositions

To prepare pharmaceutical or sterile compositions including immunoconjugates, the immunoconjugates of the invention are mixed with a pharmaceutically acceptable carrier or excipient. The compositions can additionally contain one or more other therapeutic agents that are suitable for treating or preventing cancer (including, but not limited to bladder cancer, breast cancer, colon cancer, colorectal cancer, endometrial cancer, esophageal cancer, Barrett's esophageal cancer, gastric cancer, head and neck cancer, lung cancer, multiple myeloma, ovarian cancer, liver cancer, pancreatic cancer, acute myeloid leukemia, chronic myeloid leukemia, osteosarcoma, squamous cell carcinoma, peripheral nerve sheath tumors schwannoma, glioblastoma, clear cell sarcoma of soft tissue, malignant mesothelioma, neurofibromatosis, renal cancer, melanoma, prostate cancer, benign prostatic hyperplasia (BPH), gynacomastica, and rhabdomyosarcoma).

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: tablets, Marcel Dekker, NY, 1990; Lieberman, et al. (eds.) Pharmaceutical Dosage Forms: Disperse Systems, Marcel Dekker, NY, 1990; Weiner and Kotkoskie, Excipient Toxicity and Safety, Marcel Dekker, Inc., New York, N.Y., 2000).

In one embodiment, the clinical service form (CSF) of the antibody drug conjugates of the present invention is a lyophilisate in vial containing the ADC, histidine, sucrose, and polysorbate 20. The lyophilisate can be reconstituted with water for injection, the solution comprises the ADC, histidine, sucrose, and polysorbate 20 at a pH of about 5.0. In one specific embodiment, the lyophilisate comprises 10 mg/ml of the ADC, 20 mM histidine, 240 mM sucrose, and 0.02% polysorbate 20, at pH 5.3.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, UK, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising antibodies or fragments thereof of the invention can be provided by continuous infusion, or by doses at intervals of, e.g., one day, one week, or 1-7 times per week, once every other week, once every three weeks, once every four weeks, once every five weeks, once every six weeks, once every seven weeks, or once very eight weeks. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects.

For the immunoconjugates of the invention, the dosage administered to a patient may be 0.0001 mg/kg to 100 mg/kg of the patient's body weight. The dosage may be between 0.0001 mg/kg and 30 mg/kg, 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. The dosage of the antibodies or fragments thereof of the invention may be calculated using the patient's weight in kilograms (kg) multiplied by the dose to be administered in mg/kg.

Doses of the immunoconjugates the invention may be repeated and the administrations may be separated by less than 1 day, at least 1 day, 2 days, 3 days, 5 days, 10 days, 15 days, 30 days, 45 days, 2 months, 75 days, 3 months, 4 months, 5 months, or at least 6 months. In some embodiments, the immunoconjugates of the invention may be given twice weekly, once weekly, once every two weeks, once every three weeks, once every four weeks, or less frequently. In a specific embodiment, doses of the immunoconjugates of the invention are repeated every 2 weeks.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, UK, 2001).

The route of administration may be by, e.g., topical or cutaneous application, injection or infusion by subcutaneous, intravenous, intraperitoneal, intracerebral, intramuscular, intraocular, intraarterial, intracerebrospinal, intralesional administration, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present invention may also be administered via one or more routes of administration using one or more of a variety of methods known in the art. As will be appreciated by the skilled artisan, the route and/or mode of administration will vary depending upon the desired results. Selected routes of administration for the immunoconjugates of the invention include intravenous, intramuscular, intradermal, intraperitoneal, subcutaneous, spinal or other parenteral routes of administration, for example by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the invention can be administered via a non-parenteral route, such as a topical, epidermal or mucosal route of administration, for example, intranasally, orally, vaginally, rectally, sublingually or topically. In one embodiment, the immunoconjugates of the invention is administered by infusion. In another embodiment, the immunoconjugates of the invention is administered subcutaneously.

If the immunoconjugates of the invention are administered in a controlled release or sustained release system, a pump may be used to achieve controlled or sustained release (see Langer, supra; Sefton, CRC Crit. Ref Biomed. Eng. 14:20, 1987; Buchwald et al., Surgery 88:507, 1980; Saudek et al., N. Engl. J. Med. 321:574, 1989). Polymeric materials can be used to achieve controlled or sustained release of the therapies of the invention (see e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chem. 23:61, 1983; see also Levy et al., Science 228:190, 1985; During et al., Ann. Neurol. 25:351, 1989; Howard et al., J. Neurosurg. 7 1:105, 1989; U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138, 1984).

Controlled release systems are discussed in the review by Langer, Science 249:1527-1533, 1990). Any technique known to one of skill in the art can be used to produce sustained release formulations comprising one or more immunoconjugates of the invention. See, e.g., U.S. Pat. No. 4,526,938, PCT publication WO 91/05548, PCT publication WO 96/20698, Ning et al., Radiotherapy & Oncology 39:179-189, 1996; Song et al., PDA Journal of Pharmaceutical Science & Technology 50:372-397, 1995; Cleek et al., Pro. Int'l. Symp. Control. Rel. Bioact. Mater. 24:853-854, 1997; and Lam et al., Proc. Int'l. Symp. Control Rel. Bioact. Mater. 24:759-760, 1997, each of which is incorporated herein by reference in their entirety.

If the immunoconjugates of the invention are administered topically, they can be formulated in the form of an ointment, cream, transdermal patch, lotion, gel, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the immunoconjugates are administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the immunoconjugates of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the immunoconjugates of the invention. The two or more therapies may be administered within one same patient visit.

In certain embodiments, the immunoconjugates of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The invention provides protocols for the administration of pharmaceutical composition comprising immunoconjugates of the invention alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently.

The term “concurrently” is not limited to the administration of therapies (e.g., prophylactic or therapeutic agents) at exactly the same time, but rather it is meant that a pharmaceutical composition comprising antibodies or fragments thereof the invention are administered to a subject in a sequence and within a time interval such that the antibody drug conjugates of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 5 minutes apart, less than 15 minutes apart, less than 30 minutes apart, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration. The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### EXAMPLES

### Example 1

### Generation of Antibodies

### Generation of Expression Constructs for Human, Cynomolgus Monkey, Mouse and Rat P-Cadherin Proteins

Human, mouse and rat P-cadherin extracellular domains (ECD) were gene synthesized based on amino acid sequences from the GenBank or Uniprot databases (see Table 3 below). Cynomolgus monkey P-cadherin ECD cDNA template were gene synthesized based on amino acid sequence information generated using mRNA isolated from various cyno tissues. All synthesized DNA fragments were cloned into appropriate expression vectors with C-terminal hexa-histidine tag to allow for purification.

TABLE 3
Amino Acid Sequence Information for P-cadherin

| | | | SEQ ID |
|---|---|---|---|
| Name | Description | Accession Number or Sequence | NO |
| Human | P-cadherin Human CDH3, | NM_001793.4, NP_001784 | 121 |
| (CDH3) D1-5 | residues 108-652-TAG | | |
| Rat P-cadherin | Rat CDH3, residues 100- | NM_053938.1, NP_446390 | 122 |
| (CDH3) D1-5 | 647-TAG | | |
| Mouse P-cadherin | Mouse CDH3 isoform a, | NM_001037809.5, NP_001032898 | 123 |
| (CDH3) D1-5 | residues 100-647-TAG | | |
| Cynomolgus | Cynomolgus monkey CDH3 | MKFLVNVALVFMVVYISYIYADH | 124 |
| Monkey P-cadherin | variant1, residues 108- | QTSLYKKAGFEGDRTDWVVAPIS | |
| var. 1 (CDH3) D1- | 654-TAG | VPENGKGPFPQRLNQLKSNKDRD | |
| 5 | | TKIFYSITGPGADSPPEGVFAVEKE | |
| | | TGWLLLNKPLDREEIAKYELFGHA | |
| | | VSENGASVEDPMNISIIVTDQNDH | |
| | | KPKFTQDTFRGSVLEGVLPGTSVM | |
| | | QVTATDEDDAIHTYNGVVAYSIHS | |
| | | QEPKDPHDLMFTIHRSTGTISVISS | |
| | | GLDREKVPEYTLTIQATDMDGDG | |
| | | STTTAVAVVEILDANDNAPVFDPQ | |
| | | KYESHVPENAVGHEVQRLTVTDL | |
| | | DAPNSPAWRATYLIVGGDDGDHF | |
| | | TIATHPESNQGILTTRKGLDFEAK | |
| | | NQHTLYVEVTNEAPFVLKLPTSTA | |
| | | TIVVHVEDVNEAPVFVPPSKVVEV | |
| | | QEGIPTGEAVCVYTAKDPDKENQ | |
| | | KISYRILRDPAGWLAMDPDSGQV | |
| | | TVAGTLDREDERFVRNNIYEVMV | |
| | | LAVDNGSPPTTGTGTLLLTLIDVN | |
| | | DHGPVPEPREITICNQSPESQVLNI | |
| | | TDKDLSPHTSPFQAQLTDDSDIYW | |
| | | MAEVNEKDDTVVLSLKKFLKQDT | |
| | | YDVHLSLSDHGNKEQLTVIRATV | |
| | | CDCHGHVEKCPDPWKGGGAHHH | |
| | | HHHGA | |
| Cynomolgus | Cynomolgus monkey CDH3 | MKFLVNVALVFMVVYISYIYADH | 125 |
| Monkey P-cadherin | variant2, residues 108- | QTSLYKKAGFEGDRTDWVVAPIS | |
| var. 2 (CDH3) D1- | 654-TAG | VPENGKGPFPQRLNQLKSNKDRD | |
| 5 | | TKIFYSITGPGADSPPEGVFAVEKE | |
| | | TGWLLLNKPLDREEIAKYELFGHA | |
| | | VSENGASVEDPMNISIIVTDQNDH | |
| | | KPKFTQDTFRGSVLEGVLPGTSVM | |
| | | QVTATDEDDAIHTYNGVVAYSIHS | |
| | | QEPKDPHDLMFTIHRSTGTISVISS | |
| | | GLDREKVPEYTLTIQATDMDGDG | |
| | | STTTAVAVVEILDANDNAPVFDPQ | |
| | | KYESHVPENAVGHEVQRLTVTDL | |
| | | DAPNSPAWRATYLIVGGDDGDHF | |
| | | TIATHPESNQGILTTRKGLDFEAK | |
| | | NQHTLYVEVTNEAPFVLKLPTSTA | |
| | | TIVVHVEDVNEAPVFVPPSKVVEV | |
| | | QEGIPTGEAVCVYTAKDPDKENQ | |
| | | KISYRILRDPAGWLAMDPDSGQV | |
| | | TVAGTLDREDERFVRNNIYEVMV | |
| | | LAVDNGSPPTTGTGTLLLTLIDVN | |
| | | DHGPVPEPREITICNQSPESQVLNI | |
| | | TDKDLSPHTSPFQAQLTDDSDIYW | |
| | | MAEVNEKDDTVVLSLKKFLKQGT | |
| | | YDVHLSLSDHGNKEQLTVIRATV | |
| | | CDCHGHVEKCPDPWKGGGAHHH | |
| | | HHHGA | |

### P-Cadherin Baculovirus Generation

Baculovirus expressing recombinant P-cadherin ECD proteins were generated by either the co-transfection/plaque purification method (O'Reilly et. al., 1992) or Bac-To-Bac Expression System method (Invitrogen) following manufacturer's protocol. Virus generated from the transfected insect cells was amplified using a standard low MOI infection method.

### Expression of Recombinant P-Cadherin Proteins

Suspension cultures of Tn5 cells growing in serum-free media (proprietary, in-house made recipe) were seeded at a density of 1.5e6 cells/ml and synchronously infected with recombinant P-cadherin baculovirus at either a MOI of 10 pfu/ml or a volume of 3%. The P-cadherin baculovirus culture preps were propagated in either 2 L glass Erlenmyer flasks or Wave bioreactor (GE Healthcare Life Sciences). The P-cadherin preps expressed in 2 L flasks were shaken at 120 rpm at 27° C. in serum-free media. The preps expressed in the Wave bioreactor were shaken at 25 rpm with an angle of 7.5° at 28° C. The supernatant harvested from either flasks or Wave bioreactor was harvested 2 days post-infection by centrifuging the culture at 4° C. for 10 minutes at 1800 rpm. The supernatant was then filtered with a 0.2 μM filter unit. For expression greater than 1 L, the cell culture supernatant is concentrated to 2-10× using AKTAcrossflow system (GE Healthcare Life Sciences) with KvickStart Ultrafilteration Flat sheet Cassette. The concentrated material was filtered with a 0.2 μM filter unit.

### Purification of Human, Cynomolgus Monkey, Mouse and Rat P-Cadherin ECD Proteins

Recombinant hexa-histidine tagged P-cadherin extracellular domain proteins (e.g., human pCAD-6× His, cynol pCAD-6× His, cyno2 pCAD-6× His, mouse pCAD-6× His, rat pCAD-6× His) were purified from the cell culture supernatant. The clarified supernatant was passed over an immobilized metal affinity chromatography (IMAC) on nickel Sepharose resin (GE Healthcare Life Sciences) column which had been equilibrated with 25 mM bisTrisPropane, 0.3 M NaCl, 1 mM CaCl2, pH 6.2. The supernatant is applied to an IMAC column at a flow rate of 5-8 mL/minute. After base-line washing with 25 mM bisTrisPropane, 0.3 M NaCl, 1 mM CaCl2, pH 6.2, switched to wash buffer (20 mM Tris, 0.3 M NaCl, 1 mM CaCl2, pH 7.5) for five column volumes. The pooled protein was concentrated if necessary using Amicon Ultra 15 mL centrifugal concentrators with 10 kD or 30 kD nominal molecular weight cut-offs. The pool protein was then purified by gel filtration utilizing a Superdex 200 26/60 column (GE Healthcare Life Sciences) pre-equilibrated in 20 mM Tris, 0.3 M NaCl, 1 mM CaCl2, pH 7.5. Pertinent fractions were pooled and analyzed by SDS-PAGE. Protein concentrations were determined by Bradford protein assay (Thermal Fisher).

### Immunization of Mice and Production of Hybridomas

Purified human P-cadherin ECD was diluted 1:1 with Freund's Complete Adjuvant prior to immunization of Bcl-2 transgenic mice (C57BL/6-Tgn (bcl-2) 22 wehi strain). Mice were immunized using a procedure that calls for Repetitive Immunization at Multiple Sites (RIMMS) (McIntyre G D. Hybridoma 1997). Briefly, mice were injected with 1-3 μg of antigen at 8 specific sites proximal to peripheral lymph nodes (PLN). This procedure was repeated 8 times over a 12-day period. On Day 12, a test bleed was collected and the serum antibody titer was analyzed by ELISA. Pooled PLN were removed from high titer mice on Day 15. To harvest lymphocytes, PLN were washed twice with plain DMEM and then dissociated by passage through a 0.22 micron screen (Falcon #352350). The resulting lymphocytes were washed 2 additional times prior to fusion in Cytofusion media (BTXpress Cytofusion® Electroporation Medium cat #47001). F0 myeloma cells were mixed with lymphocytes at a ratio of 4 lymphocytes to 1 FO cell. The cell mixture was centrifuged, suspended in 7 ml of Cytofusion media and subsequently added to a 9 ml electrofusion chamber (Harvard Apparatus Coaxial Chamber 9 ML Part #470020). Electrofusion was carried out per manufacturer's instructions using Cyto Pulse Sciences, Inc CEEF-50B Hybrimune/Hybridoma System. Fused cells were allowed to recover 5 min in chamber, diluted 1/10 in Fusion media without HAT (DMEM+20% FBS, Pen/Strep/Glu, 1× NEAA, 0.5× HFCS) and placed at 37° C. for one hour. 4× HAT media (DMEM+20% FBS, Pen/Strep/Glu, 1× NEAA, 4× HAT, 0.5× HFCS) was added to make a lx solution and density was adjusted to1.67×10⁴ cells/ml. The cells were then plated in 384-well plates at 60 μL/well.

### Screening of Hybridomas Secreting Antibodies to P-Cadherin

Ten days after fusion, hybridoma plates were screened for the presence of P-cadherin-specific antibodies. For the ELISA screen, Maxisorp 384-well plates (Nunc #464718) were coated with 50 μL of human P-cadherin (diluted to 15 ng/well in PBS) and incubated overnight at 4° C. The remaining protein was aspirated and wells were blocked with 1% BSA in PBS. After 30 min incubation at room temperature, the wells were washed four times with PBS+0.05% Tween (PBST). 15 μL of hybridoma supernatant was transferred to the ELISA plates. 15 μL of mouse serum, taken at the time of PLN removal, was diluted 1:1000 in PBS and added as a positive control. PBST. 50 μL of secondary antibody (goat anti mouse IgG-HRP (Jackson Immuno Research #115-035-071), diluted 1:5000 in PBS) was added to all wells on the ELISA plates. After incubation at room temperature for 1 h, the plates were washed eight times with PBST. 25 μL of TMB (KPL #50-76-05) was added and after 30 min incubation at room temperature; the plates were read at an absorbance of 605 nm. Cells from positive wells were expanded into 24-well plates in HT media (DMEM+20% FBS, Pen/Strep/Glu, 1× NEAA, 1× HT, 0.5× HFCS).

### Antibody Purification

Supernatant containing antibodies were purified using protein G (Upstate #16-266 (Billerica, Mass.)). Prior to loading the supernatant, the resin was equilibrated with 10 column volumes of PBS. Following binding of the sample, the column was washed with 10 column volumes of PBS, and the antibody was then eluted with 5 column volumes of 0.1 M Glycine, pH 2.0. Column fractions were immediately neutralized with 1/10th volume of Tris HCl, pH 9.0. The OD280 of the fractions was measured, and positive fractions were pooled and dialyzed overnight against PBS, pH 7.2.

### Humanization and Affinity Maturation of Anti-P-Cadherin Antibodies

VH and VL sequences of hybridoma derived anti-P-cadherin antibodies were humanized and affinity matured as follows.

### Generation of Humanized Sequences

DNA sequences coding for humanized VL and VH domains were ordered at GeneArt (Life Technologies Inc. Regensburg, Germany) including codon optimization for homo sapiens. Sequences coding for VL and VH domains were subcloned by cut and paste from the GeneArt derived vectors into expression vectors suitable for secretion in mammalian cells. The heavy and light chains were cloned into individual expression vectors to allow co-transfection. Elements of the expression vector include a promoter (Cytomegalovirus (CMV) enhancer-promoter), a signal sequence to facilitate secretion, a polyadenylation signal and transcription terminator (Bovine Growth Hormone (BGH) gene), an element allowing episomal replication and replication in prokaryotes (e.g. SV40 origin and ColE1 or others known in the art) and elements to allow selection (ampicillin resistance gene and zeocin marker).

### Expression and Purification of Humanized Antibodies

Human Embryonic Kidney cells constitutively expressing the SV40 large T antigen (HEK293-T ATCC11268) are one of the preferred host cell lines for transient expression of humanized and/or optimized IgG proteins. Transfection is performed using PEI (Polyethylenimine, MW 25.000 linear, Polysciences, USA Cat. No. 23966) as transfection reagent. The PEI stock solution is prepared by carefully dissolving 1 g of PEI in 900 ml cell culture grade water at room temperature (RT). To facilitate dissolution of PEI, the solution is acidified by addition of HCl to pH 3-5, followed by neutralization with NaOH to a final pH of 7.05. Finally, the volume is adjusted to 1 L and the solution is filtered through a 0.22 μm filter, aliquotted and frozen at −80° C. until further use. Once thawed, an aliquot can be re-frozen up to 3 times at −20°0 C. but should not be stored long term at −20°0 C.

HEK 293T cells are cultivated using serum-free culture medium for transfection and propagation of the cells, and ExCell VPRO serum-free culture medium (SAFC Biosciences, USA, Cat. No. 24561C) as production/feed medium. Cells prepared for transient transfections are cultivated in suspension culture. For small scale (<5 L) transfections, cells are grown in Corning shake flasks (Corning, Tewksbury, Mass.) on an orbital shaker (100-120 rpm) in a humidified incubator at 5% CO2 (seed flasks). Cells in the seed cultures should be maintained in the exponential growth phase (cell densities between 5×105 and 3×106/mL) and display a viability of >90% for transfection. Cell densities outside of this range will result in either a lag phase after dilution or reduced transfection efficiency. For small scale (<5 L) transfection an aliquot of cells is taken out of the seed cultures and adjusted to 1.4×10⁶ cells/mL in 36% of the final volume with Novartis serum-free culture medium. The DNA solution (Solution 1: 0.5 mg of heavy chain and 0.5 mg of light chain expression plasmid for a 1 L transfection) is prepared by diluting the DNA to lmg/L (final volume) in 7% of the final culture volume followed by gentle mixing. To prevent bacterial contamination, this solution is filtered using a 0.22 μm filter (e.g. Millipore Stericup). Then 3 mg/L (final volume) of PEI solution is also diluted in 7% of final culture volume and mixed gently (Solution 2). Both solutions are incubated for 5-10 min at room temperature (RT). Thereafter solution 2 is added to solution 1 with gentle mixing and incubated for another 5-15 minutes at room temperature. The transfection mix is then added to the cells and the cultivation of cells is continued for 4 to 6 hours. Finally, the remaining 50% of total production volume are achieved by addition of ExCell® VPRO serum-free culture medium. The cell cultivation is continued for eleven days post transfection. The culture is harvested by centrifugation at 4500 rpm for 20 minutes at 4° C. (Heraeus ®, Multifuge 3 S-R, Thermo Scientific, Rockford, Ill.). The cell supernatant recovered is sterile filtered through a stericup filter (0.22 μm) and stored at 4° C. until further processing.

Purification was performed on an “ÄKTA 100 explorer Air” chromatography system at 4° C. in a cooling cabinet, using a freshly sanitized (0.25 M NaOH) HiTrap ProtA MabSelect®SuRe, 5 ml column. The column was equilibrated with 5 CV of PBS (Gibco, Life Technologies, Carlsbad, Calif.), and then the sterile filtered supernatant (2 L) was loaded at 4.0 ml/min. The column was washed with 8 CV of PBS to elute the unbound sample and again washed with 5 CV of PBS. Antibody was eluted with 5 CV of 50 mM citrate, 70 mM NaCl pH 3.2. The eluate was collected in 3 ml fractions; fractions were pooled and adjusted at pH 7 with 1 M Tris HCl pH10. The pools were pooled and sterile filtered (Millipore Steriflip, 0.22 um), the OD 280 nm was measured in a Spectrophotometer ND-1000 (NanoDrop), and the protein concentration was calculated based on the sequence data. The eluate was tested for aggregation (SEC-MALS) and purity (SDS-PAGE, LAL and MS). For the second purification step, if needed, pools from the first purification were loaded into a freshly sanitised (0.5 M NaOH) SPX (Hi Load 16/60 Superdex 200 grade 120 mL (GE-Helthcare). The column was equilibrated with PBS and the run was done with PBS buffer at 1 ml/min, the eluate was collected in 1.2 ml fractions and analyzed as described for the first purification step.

Antibodies from Morphosys HuCAL PLATINUM® Phage Library Pannings

For the selection of antibodies recognizing human P-cadherin, multiple panning strategies were utilized. Therapeutic antibodies against human P-cadherin protein were generated by the selection of clones that bound to P-cadherin using as a source of antibody variant proteins a commercially available phage display library, the Morphosys HuCAL PLATINUM® library. The phagemid library is based on the HuCAL® concept (Knappik et al., 2000, J Mol Biol 296: 57-86) and employs the CysDisplay™ technology for displaying the Fab on the phage surface (WO01/05950).

For the isolation of anti-P-cadherin antibodies solid phase, liquid phase, and cell based panning strategies were employed.

### Solid Phase Panning on Recombinant P-Cadherin

Prior to the antigen selection process a coating check ELISA was performed to determine the optimal coating concentration for the antigen. Recombinant P-cadherin protein with His tag was used in the solid phase panning approach by coating on Maxisorp™ plates (Nunc) via passive adsorption. An appropriate number (dependent on the number of sub-library pools) of wells of a 96-well Maxisorp™ plate (Nunc) were coated with 125 nM antigen overnight at 4° C. The coated wells were blocked with PBS (phosphate buffered saline)/5% milk powder/5% BSA (bovine serum albumin)/0.1% Tween 20/1 mM CaCl₂. For each panning, about 50 uL HuCAL PLATINUM® phage-antibodies were blocked in solution for 2 h at room temperature (RT). After the blocking procedure, pre-blocked phage mix was added to each antigen coated and blocked well and incubated for 2 hours (h) at RT on a microtiter plate (MTP) shaker. Afterwards, unspecific bound phage was washed off by several washing steps with PBS. For elution of specifically bound phage, 25 mM DTT (Dithiothreitol) was added for 10 minutes (min) at RT. The DTT eluates were used for infection of E. coli (Escherichia coli) TG-F⁺ cells. After infection, the bacteria were plated on LB (lysogeny broth)/Cam (chloramphenicol) agar plates and incubated overnight at 30° C. Colonies were scraped off the plates and were used for phage rescue, polyclonal amplification of selected clones, and phage production. With purified phage the next panning round was started.

The second and third round of solid phase panning was performed according to the protocol of the first round except for more stringent washing conditions.

### Subcloning and Microexpression of Selected Fab Fragments

To facilitate rapid expression of soluble Fab, the Fab encoding inserts of the selected HuCAL PLATINUM® phage were subcloned from pMORPH®30 display vector into pMORPH®x11 expression vector pMORPH®x11_FH.

For initial screening and characterization an overnight culture of individual Fab-expressing E. coli clones were lysed using 0.5 mg/mL lysozyme, 0.8 mM EDTA and 4 U/μl Benzonase. Fab containing E. coli lysates were used for ELISA and FACS screening.

### ELISA Screening

Using ELISA screening, single Fab clones were identified from panning output for binding to the target antigen. Fabs are tested using Fab containing crude E. coli lysates.

For verification of Fab expression in the prepared E. coli lysates, Maxisorp™ (Nunc) 384 well plates were coated with Fd fragment specific sheep anti-human IgG diluted 1:1000 in PBS. After blocking of plates with 5% skim milk powder in PBS, Fab-containing E. coli lysates were added. Binding of Fabs was detected by F(ab)₂ specific goat anti-human IgG conjugated to alkaline phosphatase (diluted 1:5000) using Attophos fluorescence substrate (Roche, catalog #11681982001). Fluorescence emission at 535 nm was recorded with excitation at 430 nm.

For identification P-cadherin antigen binding Fab fragments Maxisorp™ (Nunc) 384 well plates were coated with 25 nM human P-cadherin antigen via passive adsorption in PBS. After blocking of plates with 5% skim milk powder in PBS, Fab-containing E. coli lysates were added. Binding of Fabs was detected by F(ab)₂ specific goat anti-human IgG conjugated to alkaline phosphatase (diluted 1:5000) using Attophos fluorescence substrate (Roche, catalog #11681982001). Fluorescence emission at 535 nm was recorded with excitation at 430 nm.

### FACS Screening (Fluorescence Activated Cell Sorting)

In FACS screening, single Fab clones binding to cell surface expressed antigen are identified from the panning output. Fabs are tested for cell binding using Fab containing crude E. coli lysates.

50 μl of cell-suspension was transferred into a fresh 96-well plate (resulting in 1×10⁵ cells/well) and mixed with 50 μl of Fab containing bacterial extracts.

The cell-antibody suspensions were then incubated on ice for 1 hour on a shaker. Following incubation, cells were spun down and washed two times with ice cold FACS buffer. After each washing step, cells were centrifuged and carefully re-suspended.

Secondary detection antibody (PE conjugated goat anti human IgG; Dianova) was added and samples were incubate on ice and subsequently washed according to Fab incubation Fluorescence intensity was determined in a FACSCalibur instrument.

### Expression and Purification of HuCAL Fab Fragments

Expression of Fab fragments was performed in E. coli TG1 F-cells. Cultures were shaken at 30° C. for 18 h. Cells were harvested and disrupted. His₆-tagged Fab fragments were isolated via IMAC and gel filtration and protein concentrations were determined by UV-spectrophotometry at 280 nm.

The identity and purity of Fab preparations was determined in native state by mass spectrometry (MS).

### Cross-Reactivity Analysis

Purified Fabs were tested in ELISA for binding to human, cyno, rat and mouse P-cadherin ECD proteins. For this purpose Maxisorp™ (Nunc) 384 well plates were coated with antigen at a concentration of 10 ug/mL in PBS overnight at 4° C. Binding of Fabs was detected by F(ab)₂ specific goat anti-human IgG conjugated to alkaline phosphatase (diluted 1:5000) using Attophos fluorescence substrate (Roche, catalog #11681982001). Fluorescence emission at 535 nm was recorded with excitation at 430 nm.

### Conversion to IgG and IgG Expression

In order to express full length IgG in HEK cells, variable domain fragments of heavy (VH) and light chains (VL) were subcloned from Fab expression vectors into appropriate pMorph®_hIg vectors for human IgG1. The cell culture supernatant was harvested 10 days post transfection. After sterile filtration, the solution was subjected to Protein A affinity chromatography using a liquid handling station. Buffer exchange was performed to 1× Dulbecco's PBS (pH 7.2, Invitrogen) and samples were sterile filtered (0.2 μm pore size). Protein concentrations were determined by UV-spectrophotometry at 280 nm and purity of IgGs was analyzed under denaturing, reducing conditions in SDS-PAGE.

### Bioassay

Anti-P-cadherin antibodies obtained following the panning processes described above were evaluated in the assay exemplified below:

### HCC1954 Cell Internalization Assay

To determine the capacity of anti-P-cadherin antibodies to undergo target mediated cell internalization a microscopy based internalization assay was established using the P-cadherin expressing HCC1954 tumor cell line.

Cells were re-suspended in full-growth medium (RPMI-1640+10% FCS and seeded into flat-bottomed microscopic 96-well assay plates (ViewPlate®-96 F TC, Perkin Elmer, #6005225) at a cell density of 5×10³ cells/well in 100 μl and incubated at 37° C. and 5% CO₂ for 2 days.

After two days, the HuCAL® antibodies (IgG) were diluted in PBS to the desired concentrations. 100 μl of the antibody solutions were added to the seeded cells and incubated for 2 h. After that, cells were washed twice with PBS, fixed with 1× CellFIX reagent (CellFIX™, BD Biosciences, #340181), washed again twice with PBS and permeabilized with 0.1% Triton X-100. Cells were then blocked with 1× Odyssey buffer (Li-Cor, No. 927-40000) for 1 h. After aspiration, cells were stained for 1 h with Hoechst (bisBenzimide H 33342 trichloride, #B2261, Sigma) and Alexa Fluor® 488 goat anti-human IgG (Invitrogen, #A-11013). After staining, cells were washed three times with PBS and analyzed using a Cellomics ArrayScan VTI HCS Reader (Thermo Fischer Scientific. To assess the half maximal internalization concentration (IC₅₀ values), IgG titration was performed covering a 10 nM to 2.4 pM range in 4 fold dilution steps.

### Removal of Post-Translational Modification (PTM) Site

One antibody, NOV169, which was identified in the internalization assay described above and found to efficiently internalize into P-cadherin expressing tumor cells HCC1954, was found to contain a single N31S PTM site in HCDR1. To prevent deamidation this site was converted into a N31Q site by single point Kunkel mutagenesis, resulting into antibody NOV169N31Q. Equivalent binding strength to recombinant human P-cadherin by NOV169N31Q in comparison to parental NOV169 was confirmed by forteBIO KD determination.

### Summary of Antibodies

Table 2 sets forth the relevant sequence information for anti-P-cadherin antibodies isolated from the Morphosys HuCAL PLATINUM® phage library and humanized anti-P-cadherin antibodies derived from murine hybridomas.

### Example 2

### X-Ray Crystallographic Structure Determination of the Human P-Cadherin EC1_EC2 and of its Complex with the NOV169N31Q Fab

The three dimensional structure of human P-cadherin was hitherto unknown. The crystal structure of a human P-cadherin ECD (extracellular domain) fragment (first two N-terminal cadherin-repeat domains, or EC1_EC2, amino acids 108 to 324, SEQ ID NO: 2, Table 2) as well as its complex with the Fab fragment of NOV (Table 2) was determined. As detailed below, human P-cadherin EC1_EC2 was expressed, refolded, purified and crystallized. In addition, purified human P-cadherin EC1_EC2 was mixed with the NOV169N31Q Fab to form a complex which was also subsequently purified and crystallized. Protein crystallography was then employed to generate atomic resolution data for human P-cadherin EC1_EC2 in the free state and bound to the NOV169N31Q Fab to define the epitope.

### Protein Production of Human P-Cadherin EC1_EC2 and NOV169N31Q Fab for Crystallography

The amino acid sequences of human P-cadherin EC1_EC2 and NOV169N31Q Fab produced for crystallography are shown in Table 4. The construct of human P-cadherin EC1_EC2 comprised residues 108 to 324 (underlined) of human P-cadherin (UniProt identifier P22223, SEQ ID NO:126), along with N-terminal residues from the recombinant expression vector (shown in lower case letters, SEQ ID NO:127). For the NOV169N31Q Fab, the amino acid sequences of the heavy and light chains are shown, along with C-terminal identification/purification tags (shown in lower case letters, SEQ ID NOs: 128 and 129, respectively).

TABLE 4
Proteins used for crystal structure determination

| | | SEQ ID |
|---|---|---|
| Construct | Amino acid sequence in one letter code | NO |
| Human P-cadherin | MGLPRGPLASLLLLQVCWLQCAASEPCRAVFREAEVTLEAGGA | 126 |
| (P22223) | EQEPGQALGKVFMGCPGQEPALFSTDNDDFTVRNGETVQERRS | |
| | LKERNPLKIFPSKRILRRHKRDWVVAPISVPENGKGPFPQRLNQ | |
| | LKSNKDRDTKIFYSITGPGADSPPEGVFAVEKETGWLLLNKPLD | |
| | REEIAKYELFGHAVSENGASVEDPMNISIIVTDQNDHKPKFTQD | |
| | TFRGSVLEGVLPGTSVMQVTATDEDDAIYTYNGVVAYSIHSQE | |
| | PKDPHDLMFTIHRSTGTISVISSGLDREKVPEYTLTIQATDMDGD | |
| | GSTTTAVAVVEILDANDNAPMFDPQKYEAHVPENAVGHEVQR | |
| | LTVTDLDAPNSPAWRATYLIMGGDDGDHFTITTHPESNQGILTT | |
| | RKGLDFEAKNQHTLYVEVTNEAPFVLKLPTSTATIVVHVEDVN | |
| | EAPVFVPPSKVVEVQEGIPTGEPVCVYTAEDPDKENQKISYRILR | |
| | DPAGWLAMDPDSGQVTAVGTLDREDEQFVRNNIYEVMVLAM | |
| | DNGSPPTTGTGTLLLTLIDVNDHGPVPEPRQITICNQSPVRQVLN | |
| | ITDKDLSPHTSPFQAQLTDDSDIYWTAEVNEEGDTVVLSLKKFL | |
| | KQDTYDVHLSLSDHGNKEQLTVIRATVCDCHGHVETCPGPWK | |
| | GGFILPVLGAVLALLFLLLVLLLLVRKKRKIKEPLLLPEDDTRDN | |
| | VFYYGEEGGGEEDQDYDITQLHRGLEARPEVVLRNDVAPTIIPT | |
| | PMYRPRPANPDEIGNFIIENLKAANTDPTAPPYDTLLVFDYEGSG | |
| | SDAASLSSLTSSASDQDQDYDYLNEWGSRFKKLADMYGGGED | |
| | D | |
| Human P-cadherin | gpDWVVAPISVPENGKGPFPQRLNQLKSNKDRDTKIFYSITGPG | 127 |
| EC1_EC2 | ADSPPEGVFAVEKETGWLLLNKPLDREEIAKYELFGHAVSENG | |
| | ASVEDPMNISIIVTDQNDHKPKFTQDTFRGSVLEGVLPGTSVMQ | |
| | VTATDEDDAIYTYNGVVAYSIHSQEPKDPHDLMFTIHRSTGTIS | |
| | VISSGLDREKVPEYTLTIQATDMDGDGSTTTAVAVVEILDANDN | |
| NOV169N31Q Fab | QVQLQQSGPGLVKPSQTLSLTCAISGDSVSSQSAAWNWIRQSPS | 128 |
| heavy chain | RGLEWLGRIYYRSKWYNDYALSVKSRITINPDTSKNQFSLQLNS | |
| | VTPEDTAVYYCARGEGYGREGFAIWGQGTLVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNH | |
| | KPSNTKVDKKVEPKSEFdykddddkgaphhhhhh | |
| NOV169N31Q Fab | DIQMTQSPSSLSASVGDRVTITCRASQTISNTLAWYQQKPGKAP | 129 |
| light chain | KLLIYAASNLQSGVPSRFSGSGSGTDFTLTISSLQPEDFATYYCQ | |
| | QYLSWFTFGQGTKVEIKRTVAAPSVFIFPPSDEQLKSGTASVVC | |
| | LLNNFYPREAKVQWKVDNALQSGNSQESVTEQDSKDSTYSLSS | |
| | TLTLSKADYEKHKVYACEVTHQGLSSPVTKSFNRGEA | |

Human P-cadherin EC1_EC2 with an N-terminal hexahistidine tag followed by a PreScission cleavage site was cloned and expressed in E. coli BL21 (DE3) Star (Invitrogen) with a pET28 vector. Following overnight induction with IPTG at 18° C., cells (67 g) were harvested and lysed with a French press in 700 ml of 50 mM TRIS pH 8.0, 500 mM NaCl, 10% glycerol, 2 mM TCEP, and 14 tablets of EDTA-free cOmplete protease inhibitor cocktail (Roche). After centrifugation (35 min at 18,000 rpm with SS34 rotor), the supernatant was sterile filtered (0.45 μm) and loaded onto a Crude FF metal chelation chromatography column (5 ml, GE Healthcare) pre-equilibrated with Buffer A (50 mM TRIS pH 8.0, 500 mM NaCl, 10% glycerol). The column was first washed with the equilibration buffer and then with buffer A including 25 mM imidazole, followed by elution with a 25 mM to 500 mM imidazole gradient. The eluted protein (36 mg) was then cleaved using PreScission protease (10 μg per mg) during overnight dialysis against 50 mM TRIS pH 8.0. After filtration (0.22 μm), the sample was loaded onto a MonoQ anion exchange chromatography column (GE Healthcare) pre-equilibrated with 50 mM TRIS pH 8.0, and eluted with a 0.0 M to 1.0 M NaCl gradient. The major peak containing P-cadherin EC1_EC2 (25.6 mg) was collected and analyzed by SDS-PAGE and HPLC. The fraction pool was then re-loaded onto the Crude FF metal chelation chromatography column (5 ml, GE Healthcare) pre-equilibrated with 50 mM TRIS pH 8.0, 500 mM NaCl, 10% glycerol as before. The P-cadherin EC1_EC2 protein was recovered in the flow-through, and analyzed by HPLC and LC-MS. LC-MS analyses showed the expected molecular weight (23,837 Da).

The NOV169N31Q Fab was expressed at 1 liter scale in E. coli. First, the plasmid encoding the Fab fragment was transformed into chemically competent TG1F⁻ E. coli cells. After overnight growth of the bacteria on LB/Agar/1% Glucose/34 μg/ml chloramphenicol plate at 37° C., one colony was used to inoculate a 6 ml pre-culture (2×YT/1.0% Glucose/34 μg/ml chloramphenicol). The culture was incubated overnight at 30° C., shaking at 220 rpm. Next day, the pre-culture was transferred to 1 liter expression culture (2×YT/0.1% Glucose/34 μg/ml chloramphenicol). The expression culture was incubated at 30° C., shaking at 220 rpm until an OD_{600 nm} of 0.6-0.8 was reached. Expression was induced by adding IPTG to a final concentration of 0.5 mM. The expression was carried on overnight at 25° C. and 220 rpm. Next day, cells were pelleted and frozen at −80° C.

The Fab fragment was purified in 2 steps using an automated protocol on the AEKTA Express system (software: Unicorn_v5.11). Bacteria pellet was first resuspended in 40 ml lysis buffer (200 mM Na phosphate pH 7.4, 0.5M NaCl, 0.1% lysozyme, 2 mM MgCl₂, 10 U/ml benzonase, 1 tablet/50 ml of cOmplete EDTA-free protease inhibitor) and incubated at room temperature for 1 hour under shaking. The cell debris was removed by centrifugation at 16,000 g for 30 min. The Fab containing supernatant was passed through 0.2 μM syringe filters (Pall, #PN4525) and loaded onto the system pre-equilibrated with running buffer (20 mM Na phosphate, 0.5M NaCl, 10 mM imidazole, pH 7.4). The first purification step was performed over a 1 ml HiTrap HP column (GE Healthcare). The column was washed with running buffer and His₆-tagged Fab fragments were eluted with the elution buffer (20 mM Na phosphate, 0.5M NaCl, 250 mM imidazole, pH 7.4). The peak fraction was automatically applied on the gel filtration column (HiLoad 16/60 Superdex 75; GE Healthcare). The purified Fab fragment was eluted in PBS. The concentration of the Fab fragment was determined by UV_{280 nm} measurements and by applying the Lambert-Beer equation, using the extinction coefficient estimated from the amino acid sequence.

### Crystallization and Structure Determination of the Human P-Cadherin EC1_EC2

Human P-cadherin EC1_EC2 was dialyzed against 10 mM Tris-HCl pH 7.4, 25 mM NaCl, concentrated to 15 mg/ml and screened for crystallization at 20° C.

Crystals were grown in 96-well SD2 plates by sitting drop vapor diffusion. In detail, 0.2 μl of protein was mixed with 0.2 μl of reservoir solution, and the drop was equilibrated against 80 μl of the same reservoir solution at 20° C. Crystals suitable for X-ray diffraction analysis were obtained with a reservoir solution made of 0.085M HEPES pH 7.5, 3,655M NaCl, 15% glycerol.

For data collection, one human P-cadherin EC1_EC2 crystal was mounted in a cryo-loop and directly flash cooled in liquid nitrogen. Diffraction data were collected at beamline X10SA (PX-II) of the Swiss Light Source (Paul Scherrer Institute, Switzerland), with a Pilatus pixel detector and X-rays of 0.99999 Å wavelength. In total, 720 images of 0.25 deg oscillation each were recorded at a crystal to detector distance of 200 mm. Data were processed and scaled at 1.40 Å resolution using XDS (Kabsch (1993) J. Appl. Crystallogr. 26:795-800) as implemented in APRV-INDEX (Kroemer, Dreyer, Wendt (2004) Acta Crystallogr. Sect. D: Biol. Crystallogr. 60:1679-1682). The crystal was in space group C2 with cell dimensions a=120.89 Å, b=76.52 Å, c=46.21 Å, alpha=90°, beta=107.79°, gamma=90°. The human P-cadherin EC1_EC2 structure was solved by molecular replacement using the program Phaser (McCoy et al., (2007) J. Appl. Cryst. 40:658-674) and PDB entry 1L3W (X. Laevis C-cadherin, 3.08 Å, 55% sequence identity). The final model was built in COOT (Emsley et al., (2010) Acta Crystallogr. Sect. D: Biol. Crystallogr. 66:486-501) and refined with Buster (Global Phasing, LTD) to R_{work} and R_{free} values of 19.8% and 21.3%, respectively, with a rmsd of 0.010 Å and 1.13° for bond lengths and bond angles, respectively.

### Human P-Cadherin EC1_EC2 Structure

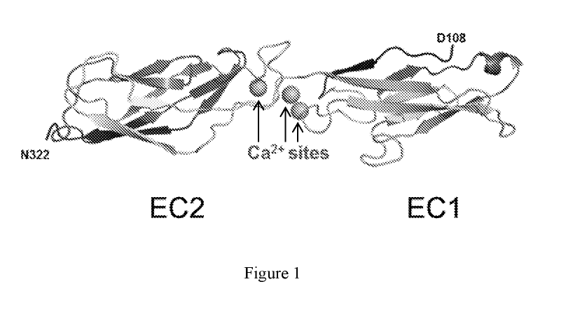

The crystal structure of human P-cadherin EC1_EC2 (amino acid residues 108 to 322) is shown in FIG. 1. Both cadherin domains had well-defined electron-density and showed the expected overall fold. Three calcium ions were observed at the domain interface.

The ECD domain of cadherins has been proposed to play a role in the extracellular architecture of adherens junctions, which control intercellular adhesion. Junction assembly involves both trans and cis homotypic interactions between the ectodomains of cadherin clusters (Boggon et al., (2002) Science 296:1308-1313; Harrison et al., (2011) Structure 19:244-256). Trans homotypic interaction involves N-terminal Trp exchange (“strand swapped dimer”) between the EC1 domains of two cadherin molecules in opposite orientation (presented by two different cells). In contrast, cis homotypic interaction involves the N-terminal extracellular cadherin (EC1) domain of one molecule and the second (EC2) domain of another molecule in the same orientation. Trans interactions are thought to be much stronger than cis interactions. While trans interactions form the molecular basis of intercellular adhesion, cis interactions are believed to promote cell adhesion via molecular clustering.

The crystal structure of the human P-cadherin EC1_EC2 fragment showed that the N-terminal segment involved in trans homotypic interactions via Trp exchange was not taking part in such interactions in the crystal and was bound to its own domain. Furthermore, an analysis of the crystal packing revealed that one of the symmetry-related P-cadherin EC1_EC2 molecule was making cis homotypic interactions highly similar to those already reported for other cadherins, showing that crystallization was, in this case, driven by cis homotypic interactions.

### Crystallization and Structure Determination of the NOV169N31Q Fab Complex

The complex of human P-cadherin EC1_EC2 with the NOV169N31Q Fab was prepared by mixing the purified human P-cadherin EC1_EC2 and the NOV169N31Q Fab at a 1.5:1.0 molar ratio (concentration measured by HPLC) and purifying the complex on a Superdex 200 (GE Healthcare) size exclusion chromatography equilibrated in 10 mM Tris-HCl pH 7.5, 150 mM NaCl, with 2 tablets of EDTA-free cOmplete protease inhibitor cocktail (Roche). Peak fractions were analyzed by SDS-PAGE and LCMS. Fractions containing the human P-cadherin EC1_EC2/NOV169N31Q Fab complex were concentrated to about 12 mg/ml, CaCl₂ was added to a final concentration of 5 mM and the sample was screened for crystallization at 20° C.

Crystals were grown in 96-well SD2 plates by sitting drop vapor diffusion. In detail, 0.2 μl of protein was mixed with 0.2 μl of reservoir solution, and the drop was equilibrated against 80 μl of the same reservoir solution at 20° C. Crystals suitable for X-ray diffraction analysis were obtained with a reservoir solution made of 0.2M calcium acetate, 10% (w/v) PEG 8,000, 0.1M MES pH 6.5.

Before data collection, one human P-cadherin EC1_EC2/NOV169N31Q Fab crystal was briefly transferred into a 1:1 mix of the reservoir solution with 20% PEG 8,000, 30% glycerol, and flash cooled in liquid nitrogen.

Diffraction data were collected at beamline X10SA (PX-II) of the Swiss Light Source (Paul Scherrer Institute, Switzerland), with a Pilatus pixel detector and X-rays of 0.99999 Å wavelength. In total, 720 images of 0.25 deg oscillation each were recorded at a crystal to detector distance of 340 mm. Data were processed and scaled at 2.10 Å resolution using XDS (Kabsch (1993) J. Appl. Crystallogr. 26:795-800) as implemented in APRV-INDEX (Kroemer, Dreyer, Wendt (2004) Acta Crystallogr. Sect. D: Biol. Crystallogr. 60:1679-1682). The crystal was in space group P2₁2₁2 with cell dimensions a=172.69 Å, b=77.79 Å, c=133.41 Å, alpha=90°, beta=90.0°, gamma=90°. The human P-cadherin EC1_EC2/NOV169N31Q Fab complex structure was solved by molecular replacement using Phaser (McCoy et al., (2007) J. Appl. Cryst. 40:658-674). The final model was built in COOT (Emsley et al., (2010) Acta Crystallogr. Sect. D: Biol. Crystallogr. 66:486-501) and refined with Buster (Global Phasing, LTD) to R_{work} and R_{free} values of 19.2% and 22.1%, respectively, with a rmsd of 0.010 Å and 1.18° for bond lengths and bond angles, respectively. Residues of human P-cadherin EC1_EC2 that contain atoms within 4.0 Å of any atom in NOV169N31Q Fab were identified by the program Ncont of the CCP4 program suite (Collaborative Computing Project, Number 4 (1994) Acta Crystallogr. Sect. D: Biol. Crystallogr. 50:760-763) and listed in Tables 4 and 5. Residues of human P-cadherin EC1_EC2 that become less accessible to solvent upon binding of the NOV169N31Q antibody were identified by the program AREAIMOL of the CCP4 program suite.

### P-Cadherin EC1_EC2 Epitope for NOV169N31Q

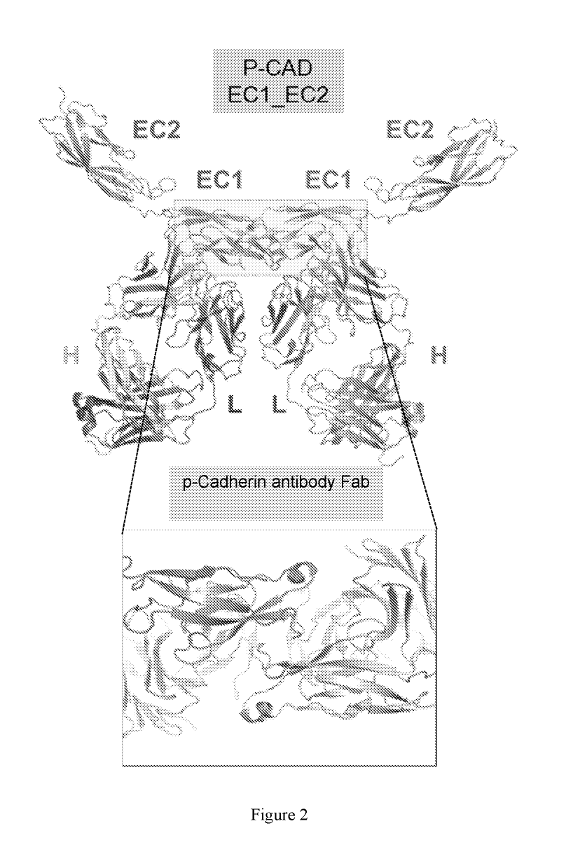

The crystal structure of the P-cadherin EC1_EC2/NOV169N31Q Fab complex was used to identify the P-cadherin EC1_EC2 epitope for NOV169N31Q. The X-ray analysis shows that NOV169N31Q binds to the EC1 domain (N-terminal cadherin-repeat domain) of human P-cadherin (FIG. 2). There are two copies of the NOV169N31Q Fab-human P-cadherin EC1_EC2 complex in the asymmetric unit of the crystal (an asymmetric unit contains all the structural information which is needed to reproduce the whole crystal by applying crystallographic symmetry operators). Both copies share almost identical residues in contact with NOV169N31Q Fab except for small variations due to crystal packing.

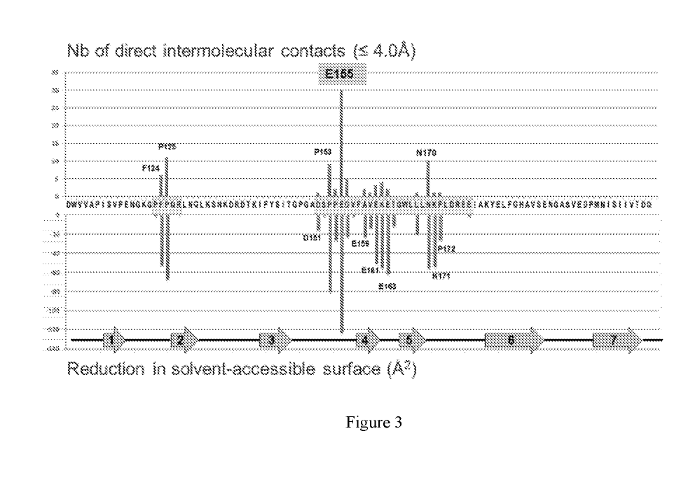

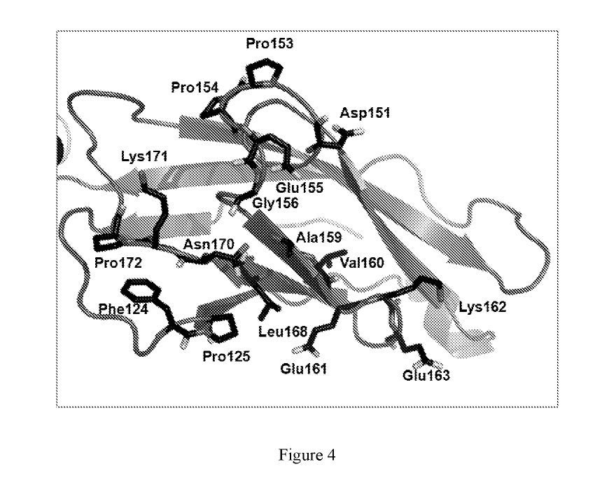

The interaction surface on human P-cadherin EC1_EC2 by the NOV169N31Q Fab is formed by two discontinuous (i.e., noncontiguous) sequences, entirely comprised within the EC1 domain of P-cadherin and encompassing residues 123 through 127, and residues 151 through 177 (FIG. 3). Among those, residues 124 and 125, and residues 151 through 172 are contributing direct intermolecular contacts shorter than 4.0 Å (between non-hydrogen atoms), as detailed in Tables 4 and 5 and shown in FIG. 3. These residues form the three-dimensional surface that is recognized by the NOV169N31Q Fab (FIG. 4).

TABLE 5
Interactions between human P-cadherin EC1_EC2
and the NOV169N31Q Fab heavy chain (H).
P-cadherin residues are numbered based upon P22223
(SEQ ID NO:126). Fab heavy chain residues are
numbered based upon their linear amino acid sequence
(SEQ ID NO:128). P-cadherin residues shown
have at least one atom within 4.0 Å of an atom in
the NOV169N31Q Fab.

| Human | | | | |
|---|---|---|---|---|
| P-cadherin | | NOV169N31Q | | |
| Residues | | Fab Residues | | |
| (SEQ ID NO:126) | | (SEQ ID NO:128) | | |
| Amino | | Amino | | |
| acid | Number | acid | Number | Chain |
| Phe | 124 | Leu | 65 | H |
| Asp | 151 | Tyr | 105 | H |
| Pro | 153 | Tyr | 54 | H |
| | | Arg | 56 | H |
| | | Tyr | 60 | H |
| | | Tyr | 105 | H |
| Pro | 154 | Tyr | 54 | H |
| Glu | 155 | Arg | 52 | H |
| | | Tyr | 54 | H |
| | | Tyr | 105 | H |
| | | Arg | 107 | H |
| Gly | 156 | Arg | 107 | H |
| Pro | 172 | Leu | 65 | H |

TABLE 6
Interactions between human P-cadherin EC1_EC2
and the NOV169N31Q Fab light chain (L).
P-cadherin residues are numbered based upon
P22223 (SEQ ID NO:126). Fab light chain
residues are numbered based upon their linear amino
acid sequence (SEQ ID NO:129). P-cadherin
residues shown have at least one atom within 4.0 Å
of an atom in the NOV169N31Q Fab.

| Human | | | | |
|---|---|---|---|---|
| P-cadherin | | NOV169N31Q | | |
| Residues | | Fab Residues | | |
| (SEQ ID NO:126) | | (SEQ ID NO:129) | | |
| Residue | Number | Residue | Number | Chain |
| Phe | 124 | Asp | 1 | L |
| Pro | 125 | Ile | 2 | L |
| | | Gln | 27 | L |
| | | Ser | 93 | L |
| Glu | 155 | Trp | 94 | L |
| Gly | 156 | Trp | 94 | L |
| Ala | 159 | Leu | 92 | L |
| Val | 160 | Leu | 92 | L |
| Glu | 161 | Gln | 27 | L |
| | | Thr | 28 | L |
| Lys | 162 | Ser | 30 | L |
| Glu | 163 | Gly | 68 | L |
| Leu | 168 | Leu | 92 | L |
| Asn | 170 | Ser | 93 | L |
| | | Trp | 94 | L |
| Lys | 171 | Trp | 94 | L |

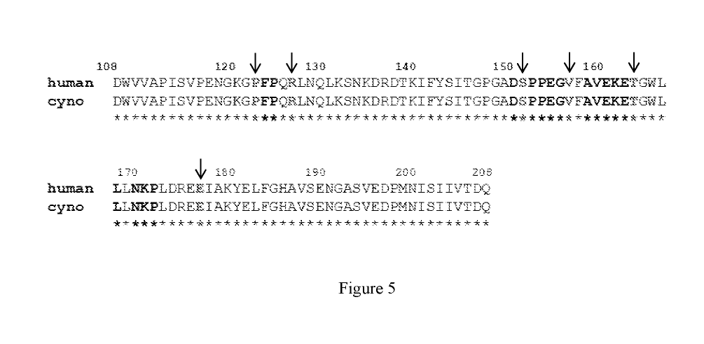

In contrast to the other extracellular cadherin-repeat domains of human P-cadherin, the EC1 domain does not harbor any known N-linked or O-linked glycosylation sites. NOV169N31Q binding to P-cadherin is thus independent of glycosylation. Also worth of note, the amino acid sequence of the human P-cadherin EC1 domain is fully conserved in cynomolgus (Macaca fascicularis) P-cadherin (FIG. 5). Therefore, the P-cadherin epitope recognized by NOV169N31Q is fully conserved in this monkey species used in toxicological studies.

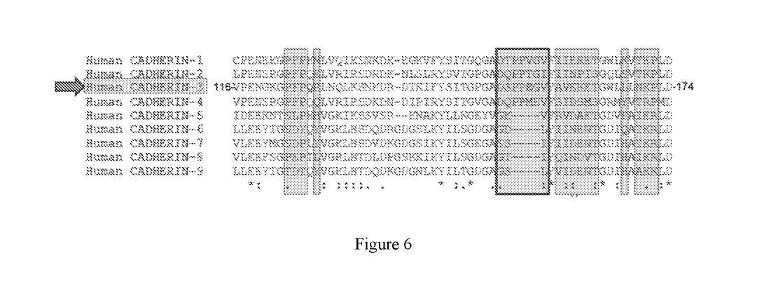

Glu155 of human P-cadherin EC1_EC2 is the epitope residue making most contacts with the NOV169N31Q Fab (see FIG. 3). Interestingly, Glu155 is located within a non-conserved insertion found in human cadherins 1 to 4 only, as shown by a multiple sequence alignment of all human cadherins (FIG. 6). As Glu155 itself is not conserved in human cadherins 1, 2 and 4, NOV169N31Q is expected to display high selectivity towards human cadherin-3 (aka human P-cadherin).

As already mentioned above, cadherins plays an important role in the molecular mechanism of intercellular adhesion, which involves both strong trans and weak cis homotypic interactions between the ectodomains of cadherin clusters. (Boggon et al., (2002) Science 296:1308-1313; Harrison et al., (2011) Structure 19:244-256). Based on the crystal structure of the human P-cadherin EC1_EC2/NOV169N31Q Fab complex, it appears that the binding epitope for NOV169N31Q partially overlaps with the surface region of the EC1 domain involved in cis homotypic interactions, but not with the N-terminal region involved in trans (intercellular) homotypic interactions. As a consequence, NOV does not compete with strong trans interactions for cadherin binding, and therefore is more likely to have easier access to its binding epitope. Moreover, the binding of this antibody to its target antigen is not expected to disrupt intercellular adhesion fully, as trans homotypic interactions are preserved.

### Example 3

### Generation of Drug Moieties (Payloads) and Linker-Payloads

### Example 3-i

### Synthetic Procedure for Intermediates

### Synthesis of (S)-2-phenyl-1-(5-phenyl-1,3,4-oxadiazol-2-yl)ethanamine (i-1)

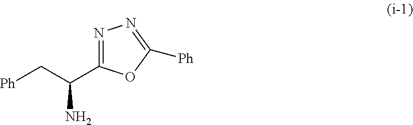 [SMILES: N[C@@H](Cc1ccccc1)c1nnc(-c2ccccc2)o1]

Step 1: (S)-2-((t-Butoxycarbonyl)amino)-3-phenylpropanoic acid (200 mg, 0.754 mmol) was added to dichloromethane (5.5 ml) at 0° C., followed by carbonyldiimidazole (128 mg, 0.792 mmol). After stirring at 0° C. for 30 min, benzohydrazide (103 mg, 0.754 mmol) was added. After additional 45 min at 0° C., carbon tetrabromide (497 mg, 1.5 mmol) and triphenylphosphine (198 mg, 0.754 mmol) were added. The mixture was stirred for 2 h at 0° C. and then at rt for 16 h. Water was added to the mixture and extracted with DCM (5 ml×3). The organic layers were combined, dried with Na₂SO₄, filtered and concentrated. The crude product was purified by a silica gel column (20-40% ethyl acetate in hexanes) to obtain t-butyl [(1S)-2-phenyl-1-(5-phenyl-1,3,4-oxadiazol-2-yl) ethyl] carbamate. MS m/z 366 (M+H). Retention time 1.351 min. 1H NMR (400 MHz, Chloroform-d) δ 8.03-7.85 (m, 2H), 7.62-7.38 (m, 3H), 7.33-7.16 (m, 3H), 7.18-7.04 (m, 2H), 5.35 (d, J=7.9 Hz, 1H), 5.15 (d, J=9.1 Hz, 1H), 3.28 (d, J=6.6 Hz, 2H), 1.54 (s, 9H).

Step 2: To the compound obtained in step 1, (548 mg, 1.5 mmol) in DCM (5 ml) was added TFA (1.5 ml). The resulting solution was stirred at room temperature for 18 h and then concentrated to obtain (S)-2-phenyl-1-(5-phenyl-1,3,4-oxadiazol-2-yl)ethanamine (i-1) TFA salt. It was used without further purification. MS m/z 266 (M+H). Retention time 0.858 min.

### Synthesis of 2-phenyl-1-(pyrimidin-2-yl)ethanamine (i-2)

Benzylmagnesium chloride (1.2 ml, 2.4 mmol) (2M in THF) was added dropwise to 2-cyanopyrimidine (210 mg, 2.00 mmol) in toluene (10 ml) at 0° C. The reaction was stirred at this temperature for 1 h. Then 2-butanol (10 ml) was added, followed by sodium borohydride (106 mg, 2.80 mmol). The reaction was stirred at rt for 1 h, and then quenched with MeOH (3 ml) and water. The mixture was extracted with EtOAc (2×30 ml). The organic layer was dried with Na₂SO₄, filtered and concentrated. The crude product was purified by preparative HPLC (10-30% acetonitrile in water with 0.05% TFA) to obtain 2-phenyl-1-(pyrimidin-2-yl)ethanamine (i-2). MS m/z 200.2 (M+H). Retention time 0.637 min. ¹H NMR (400 MHz, Acetonitrile-d3) δ 8.75 (d, J=5.0 Hz, 2H), 7.41 (t, J=4.9 Hz, 1H), 7.27 (m, 3H), 7.14-7.05 (m, 2H), 4.84 (t, J=6.7 Hz, 1H), 3.45 (dd, J=14.1, 6.3 Hz, 1H), 3.33 (dd, J=14.1, 7.1 Hz, 1H).

### Synthesis of (S)-2-phenyl-1-(1H-pyrazol-3-yl)ethanamine (i-3)

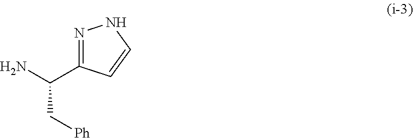 [SMILES: N[C@@H](Cc1ccccc1)c1cc[nH]n1]

Step 1: Hydrazine monohydrate (0.034 ml, 0.69 mmol) was added to (S,E)-t-butyl (5-(diethylamino)-3-oxo-1-phenylpent-4-en-2-yl)carbamate (60 mg, 0.17 mmol) in MeOH (5 ml). The reaction was heated at 70° C. for 2 h and then 50° C. for 3 days. The reaction mixture was concentrated, taken up in water, and extracted with DCM (5 ml×2). The DCM layers were combined, dried with Na₂SO₄, filtered and concentrated. The residue was purified by silica gel preparative TLC (4% MeOH in DCM) to obtain (S)-t-butyl (2-phenyl-1-(1H-pyrazol-3-yl)ethyl)carbamate. MS m/z 288.2 (M+H). Retention time 1.310 min. 1H NMR (400 MHz, Chloroform-d) δ 7.53-7.35 (m, 1H), 7.28-6.90 (m, 5H), 6.01 (s, 1H), 5.47-5.25 (m, 0.3 H), 5.15-4.84 (m, 0.7H), 3.40 (s, 1H), 3.09 (d, J=8.0 Hz, 2H), 1.34 (d, J=31.2 Hz, 9H).

Step 2: A solution of the compound obtained in step 1 (38 mg, 0.13 mmol) in DCM (2 ml) was treated with TFA (0.5 ml) at rt for 2 h and then concentrated to give (S)-2-phenyl-1-(1H-pyrazol-3-yl)ethanamine TFA salt (i-3). The product was used in the next step without further purification. MS m/z 188.2 (M+H). Retention time 0.616 min.

### Synthesis of (S)-t-butyl (3-(2-amino-3-hydroxypropyl)phenyl)carbamate (i-4)

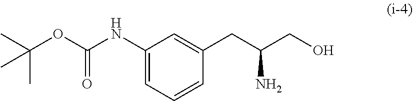 [SMILES: CC(C)(C)OC(=O)Nc1cccc(C[C@H](N)CO)c1]

Step 1: BH₃ in THF (1M, 10 ml) was added to (S)-2-((t-butoxycarbonyl)amino)-3-(3-nitrophenyl)propanoic acid (562 mg, 1.81 mmol) in THF (10 ml) with stirring at 0° C. Then the reaction was stirred at 50° C. for 1 h. The reaction mixture was cooled at 0° C., quenched with water, diluted with EtOAc and washed with 10% aqueous K₂CO₃, dried over MgSO4, filtered and concentrated. The crude was purified by a silica gel column (30-70% EtOAc-hexanes) to obtain (S)-t-butyl (1-hydroxy-3-(3-nitrophenyl)propan-2-yl)carbamate as white solid. MS m/z 319.1 (M+Na). Retention time 1.183 minute. 1H NMR (600 MHz, Chloroform-d) δ 8.13-8.04 (m, 2H), 7.57 (d, J=7.7 Hz, 1H), 7.46 (dd, J=8.9, 7.6 Hz, 1H), 4.76 (s, 1H), 3.87 (dq, J=8.0, 4.6, 4.1 Hz, 1H), 3.69 (dd, J=10.9, 3.9 Hz, 1H), 3.58 (dd, J=10.8, 4.7 Hz, 1H), 2.97 (td, J=13.1, 12.5, 7.3 Hz, 2H), 1.37 (s, 9H).

Step 2: To (S)-t-butyl (1-hydroxy-3-(3-nitrophenyl)propan-2-yl)carbamate (0.31 g, 1.0 mmol) in acetonitrile (5 ml) was added 10% hydrochloric acid (5 ml). The reaction mixture was stirred at rt for 48 h and then concentrated to give (S)-2-amino-3-(3-nitrophenyl)propan-1-ol as HCl salt. MS m/z 197.2 (M+H). Retention time 0.775 min.

Step 3: (S)-2-Amino-3-(3-nitrophenyl)propan-1-ol HCl salt (0.243 g, 1.046 mmol) was dissolved in MeOH (10 ml) and 10% palladium on carbon (50 mg, 0.047 mmol) was added. A 2 L hydrogen balloon was attached. The reaction was flushed with H₂ three times and then stirred at rt for 1 h. LCMS indicated the reaction was complete. The reaction was filtered through a celite pad and concentrated to give (S)-2-amino-3-(3-aminophenyl)propan-1-ol as HCl salt. MS m/z 167.2 (M+H). Retention time 0.373 min.

Step 4: (S)-2-Amino-3-(3-aminophenyl)propan-1-ol HCl salt (0.212 g, 1.046 mmol) and Boc₂O (228 mg, 1.05 mmol) and dioxane-water-AcOH (10:9:1, 20 ml) were combined and stirred at rt for 3 days. LCMS indicated the reaction was 75% complete. Additional Boc₂O (150 mg) was added and the reaction was further stirred for 6 h. The reaction mixture was then concentrated and purified with preparative HPLC (10-40% acetonitrile in water with 0.05% TFA) to give (S)-t-butyl (3-(2-amino-3-hydroxypropyl)phenyl)carbamate (i-4) as an oil. MS m/z 267.2 (M+H). Retention time 1.011 min.

### Synthesis of (S)-t-butyl (4-(2-amino-3-hydroxypropyl)phenyl)carbamate (i-5)

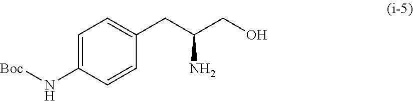 [SMILES: CC(C)(C)OC(=O)Nc1ccc(C[C@H](N)CO)cc1]

Step 1: To (S)-2-((t-butoxycarbonyl)amino)-3-(4-nitrophenyl)propanoic acid (0.80 g, 2.58 mmol) in THF (10 ml) was added borane dimethyl sulfide complex (1.00 ml, 10.5 mmol) at 0° C. The reaction was stirred for 10 min at 0° C. and then at rt for 5 h. The reaction was then quenched with water at 0° C. The quenched mixture was partitioned between DCM and 1M aqueous Na₂CO₃. The DCM layer was separated, dried over Na₂SO₄, filtered and concentrated to give (S)-t-butyl (1-hydroxy-3-(4-nitrophenyl)propan-2-yl)carbamate as white solid. MS m/z 319.1 (M+Na). Retention time 1.031 min. 1H NMR (400 MHz, Chloroform-d) δ 8.10 (d, J=8.8 Hz, 2H), 7.33 (d, J=8.7 Hz, 2H), 4.73 (s, 1H), 3.83 (s, 1H), 3.70-3.56 (m, 1H), 3.50 (d, J=4.6 Hz, 1H), 2.91 (d, J=7.1 Hz, 2H), 1.32 (s, 9H).

Step 2: (S)-t-Butyl (1-hydroxy-3-(4-nitrophenyl)propan-2-yl)carbamate (300 mg, 1.01 mmol) in acetonitrile (5 ml) and 10% hydrochloric acid (5 ml) was stirred at rt for 4 h and then concentrated. The residue was treated with saturated aqueous Na₂CO₃, and extracted with DCM-iPrOH (10:1, 10 ml×3). The organic layers were combined, dried and concentrated to give (S)-2-amino-3-(4-nitrophenyl)propan-1-ol. MS m/z 197.2 (M+H). Retention time 0.512 min. ¹H NMR (400 MHz, Chloroform-d) □8.32-7.92 (m, 2H), 7.41-7.21 (m, 2H), 4.18-4.00 (m, 1H), 3.66-3.49 (m, 2H), 3.49-3.36 (m, 1H), 3.25-3.00 (m, 1H), 3.01-2.74 (m, 2H), 2.70-2.65 (m, 1H).

Step 3: (S)-2-Amino-3-(4-nitrophenyl)propan-1-ol (200 mg, 1.019 mmol) was dissolved in MeOH (10 ml) and 10% Pd/C (50 mg) was added. A 2 L hydrogen balloon was attached. The reaction was flushed with H2 three times and then stirred at rt for 3 h. The reaction mixture was filtered through a celite pad and then concentrated to give (S)-2-amino-3-(4-aminophenyl)propan-1-ol. MS m/z 167.2 (M+H). Retention time 0.240 min.

Step 4: (S)-2-Amino-3-(4-aminophenyl)propan-1-ol (168 mg, 1.012 mmol) was dissolved in dioxane (10 ml)-water (9 ml)-AcOH (1 ml) and t-butyl dicarbonate (0.28 g, 1.28 mmol) were combined and stirred at rt for 16 h. The reaction mixture was then concentrated and purified with ISCO using C18 column, eluted with 10-40% acetonitrile in water with 0.05% TFA to give (S)-t-butyl (4-(2-amino-3-hydroxypropyl)phenyl)carbamate TFA (i-5). MS m/z 267.2 (M+H). Retention time 0.764 min. ¹H NMR (400 MHz, Acetonitrile-d3) □7.59 (s, 1H), 7.36 (d, J=8.5 Hz, 2H), 7.16 (d, J=8.5 Hz, 2H), 6.14 (s, 3H), 3.69 (dd, J=11.7, 3.2 Hz, 1H), 3.57-3.36 (m, 2H), 2.86 (d, J=7.1 Hz, 2H), 1.47 (s, 9H).

### Synthesis of (S)-t-butyl (3-(2-amino-3-methoxypropyl)phenyl)carbamate (i-6)

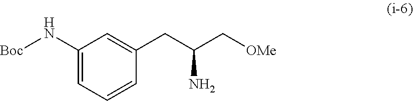 [SMILES: COC[C@@H](N)Cc1cccc(NC(=O)OC(C)(C)C)c1]

Step 1: Borane dimethyl sulfide complex (3.00 ml, 31.6 mmol) was added to (S)-2-((t-butoxycarbonyl)amino)-3-(3-nitrophenyl)propanoic acid (1.5 g, 4.83 mmol) in THF (10 ml) at 0° C. The reaction was stirred for 10 min at 0° C. and then at rt for 6 h. The reaction was then quenched with water at 0° C. The quenched reaction mixture was partitioned between DCM and 1M aqueous Na₂CO₃. The DCM layer was separated, dried over Na₂SO₄, filtered and concentrated to give (S)-t-butyl (1-hydroxy-3-(3-nitrophenyl)propan-2-yl)carbamate as white solid. MS m/z 319.1 (M+Na). Retention time 1.031 min. ¹H NMR (400 MHz, Chloroform-d) □8.14-7.97 (m, 2H), 7.57 (dt, J=7.7, 1.4 Hz, 1H), 7.46 (dd, J=8.8, 7.6 Hz, 1H), 4.77 (d, J=14.5 Hz, 1H), 3.87 (s, 1H), 3.69 (dd, J=10.9, 3.8 Hz, 1H), 3.58 (dd, J=10.9, 4.7 Hz, 1H), 2.95 (t, J=6.8 Hz, 2H), 1.37 (s, 9H).

Step 2: To (S)-t-butyl (1-hydroxy-3-(3-nitrophenyl)propan-2-yl)carbamate (0.200 g, 0.675 mmol) in THF/DMF 4:1 (10 ml) at 0° C. was added NaH (60% in mineral oil ,0.048 g, 1.2 mmol) slowly, followed by methyl iodide (0.19 g, 1.3 mmol). The resulting mixture was stirred at rt for 1 h. The reaction was quenched carefully by slow addition of water until no bubbling (H₂) was observed. The crude product was extracted with EtOAc (10 ml×3). The combined organic phases was dried over Na₂SO₄, filtered and concentrated. The residue was purified by ISCO using C18 column and eluted with 30-67% ACN in water with 0.05% TFA to give (S)-t-butyl (1-methoxy-3-(3-nitrophenyl)propan-2-yl)carbamate as white solid. MS m/z 333.1 (M+Na). Retention time 1.205 min. ¹H NMR (400 MHz, Chloroform-d) d 8.10 (dd, J=4.5, 2.5 Hz, 2H), 7.59 (d, J=7.5 Hz, 1H), 7.48 (dd, J=8.8, 7.6 Hz, 1H), 4.96 (d, J=8.7 Hz, 1H), 4.07-3.88 (m, 1H), 3.43-3.28 (m, 5H), 2.98 (d, J=7.2 Hz, 2H), 1.40 (s, 9H).

Step 3: (S)-t-Butyl (1-methoxy-3-(3-nitrophenyl)propan-2-yl)carbamate (124 mg, 0.400 mmol) in acetonitrile (3 ml) and 10% hydrochloric acid (3 ml) was stirred at rt for 4 h and then concentrated. Saturated aqueous Na₂CO₃ was added to the residue and the resulting mixture was extracted with DCM-iPrOH (10:1, 10 ml×3). The organic layers were combined, dried and concentrated to give (S)-1-methoxy-3-(3-nitrophenyl)propan-2-amine. MS m/z 211.2 (M+H). Retention time 0.622 min.

Step 4: (S)-1-Methoxy-3-(3-nitrophenyl)propan-2-amine was dissolved in MeOH (10 ml) and 10% Pd/C (50 mg) was added. A 2 L hydrogen balloon was attached. The reaction was flushed with H₂ three times and then stirred at rt for 3 h. The reaction mixture was filtered through a celite pad and then concentrated to give (S)-3-(2-amino-3-methoxypropyl)aniline. MS m/z 181.2 (M+H). Retention time 0.282 min.

Step 5: (S)-3-(2-Amino-3-methoxypropyl)aniline (62.6 mg, 0.347 mmol) in dioxane (3 ml)-water (3 ml)-AcOH (0.6 ml) and t-butyl dicarbonate (0.093 ml, 0.4 mmol) were combined and stirred at rt for 16 h. The reaction mixture was then concentrated and purified with ISCO using C18 column, eluted with 10-40% acetonitrile in water with 0.05% TFA to give (S)-t-butyl (3-(2-amino-3-methoxypropyl)phenyl)carbamate TFA salt (i-6). MS m/z 281.2 (M+H). Retention time 0.856 min. ¹H NMR (400 MHz, Acetonitrile-d3) d 7.61 (s, 1H), 7.40-7.12 (m, 3H), 6.89 (dt, J=7.4, 1.5 Hz, 1H), 6.78 (s, 3H), 3.59 (m, 1H), 3.50 (dd, J=10.6, 3.4 Hz, 1H), 3.38 (dd, J=10.6, 6.9 Hz, 1H), 3.33 (s, 3H), 2.90 (d, J=7.5 Hz, 3H), 1.48 (s, 9H).

### Synthesis of (3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoic acid (i-7)

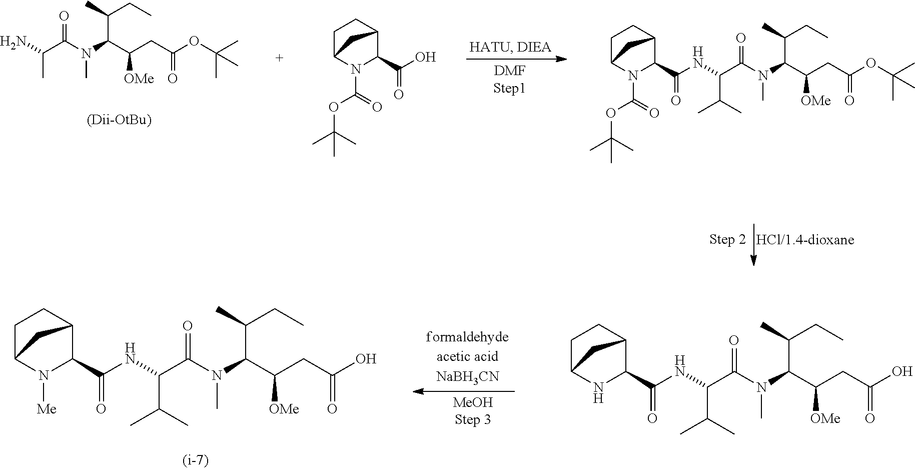

Step 1: Dil-OtBu HCl salt (388 mg, 0.982 mmol), (1R,3S,4S)-2-(t-butoxycarbonyl)-2-azabicyclo[2.2.1]heptane-3-carboxylic acid (287 mg, 1.19 mmol), HATU (411 mg, 1.08 mmol) and DIEA (0.42 ml, 2.38 mmol) and DMF (5 ml) were combined and stirred at rt for 30 min. The reaction mixture was diluted with water (10 ml) and purified by RP-C18 ISCO to give (3R,4S,5S)-t-butyl 4-((S)—N,3-dimethyl-2-((1R,3S,4S)-2-methyl-2-azabicyclo[2.2.1]heptane-3-carboxamido)butanamido)-3-methoxy-5-methylheptanoate. MS (m+1)=582.5, HPLC Peak RT=1.542 min

Step 2: The product obtained in step 1 (540 mg, 0.93 mmol) in 4M HCl in 1.4-dioxane (10 ml) was stirred at rt overnight. The reaction mixture was concentrated in to give (3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoic acid. MS (m+1)=426.2, HPLC Peak RT=0.736 min

Step 3: The product obtained in step 2 (430 mg, 0.93 mmol), 37% formaldehyde solution (0.38 ml, 4.7 mmol), acetic acid (0.27 ml, 4.65 mmol), NaBH3CN (585 mg, 9.31 mmol) and MeOH (10 ml) were combined and stirred at rt for 30 min and then concentrated. The residue was purified by RP-C18 ISCO to give 450 mg of (3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoic acid as a TFA salt. The TFA salt was treated with 10 ml of 12N HCl solution and concentrated twice to give (3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoic acid HCl salt (i-7). MS (m+1)=440.2, HPLC Peak RT=0.754 min

### Synthesis of Boc-Dap-OMe: ((S)-tert-butyl 2-((1R,2R)-1,3-dimethoxy-2-methyl-3-oxopropyl)pyrrolidine-1-carboxylate) (i-8)

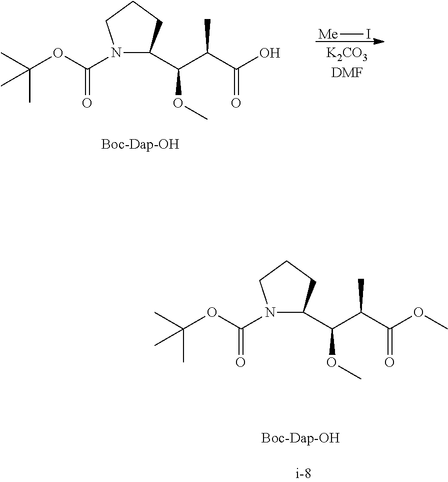 [SMILES: CO[C@H]([C@@H](C)C(=O)O)[C@@H]1CCCN1C(=O)OC(C)(C)C ; CI ; COC(=O)[C@H](C)[C@@H](OC)[C@@H]1CCCN1C(=O)OC(C)(C)C]

Boc-Dap-OH (Small Molecules Inc., 3.11 g, 10.8 mmol), K2CO3 (2.99 g, 21.6 mmol), iodomethane (2.95 g) and acetone (55 mL) were combined. The reaction was stirred at 20° C. for 2 h. An additonal methyliodide (2.28 g) was added to the reaction and the reaction was stirred at 40° C. for 3 h. The reaction mixture was concentrated. The residue was partitioned between 200 mL EtOAc and 100 mL H2O. The organic layer was separated, washed with 50 mL saturated aq NaCl, dryed over MgSO₄, filtered and concentrated, affording Boc-Dap-OMe (i-8) as a yellow oil. MS (ESI+) m/z calc 324.2, found 324.2 (M+23). Retention time 1.245 min.

### Sythesis of Dap-OMe: ((2R,3R)-methyl 3-methoxy-2-methyl-3-((S)-pyrrolidin-2-yl)propanoate) (i-9)

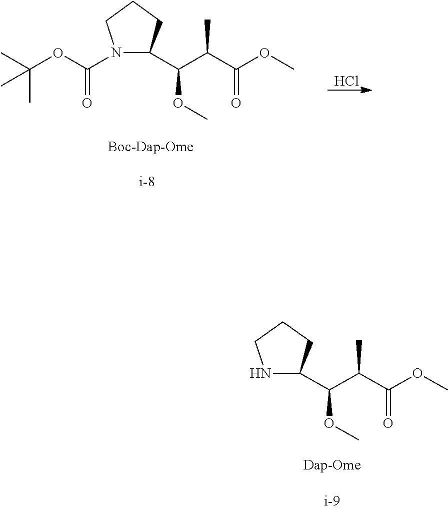 [SMILES: COC(=O)[C@H](C)[C@@H](OC)[C@@H]1CCCN1C(=O)OC(C)(C)C ; Cl ; COC(=O)[C@H](C)[C@@H](OC)[C@@H]1CCCN1]

Boc-Dap-OMe (3.107 g, 10.3 mmol) was combined with HCl in diethyl ether (2 M, 10 mL) and concentrated. This operation was repeated. The reaction was complete after the 7^{th} treatment. HCl salt of Dap-OMe (i-9) was obtained as a white solid after being concentrated. MS (ESI+) m/z calc 202.1, found 202.2 (M+1). Retention time 0.486 min. ¹H NMR (400 MHz, CDCl₃): δ 4.065-4.041 (m, 1H), 3.732 (br.s, 1H), 3.706 (s, 3H), 3.615 (s, 3H), 3.368 (br.s, 1H), 3.314 (br.s, 1H), 2.795 (q, 1H, J=6.8 Hz), 2.085-1.900 (m, 4H), 1.287 (d, 3H, J=7.2 Hz).

### Example 3-1

### (S)-Methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate (1)

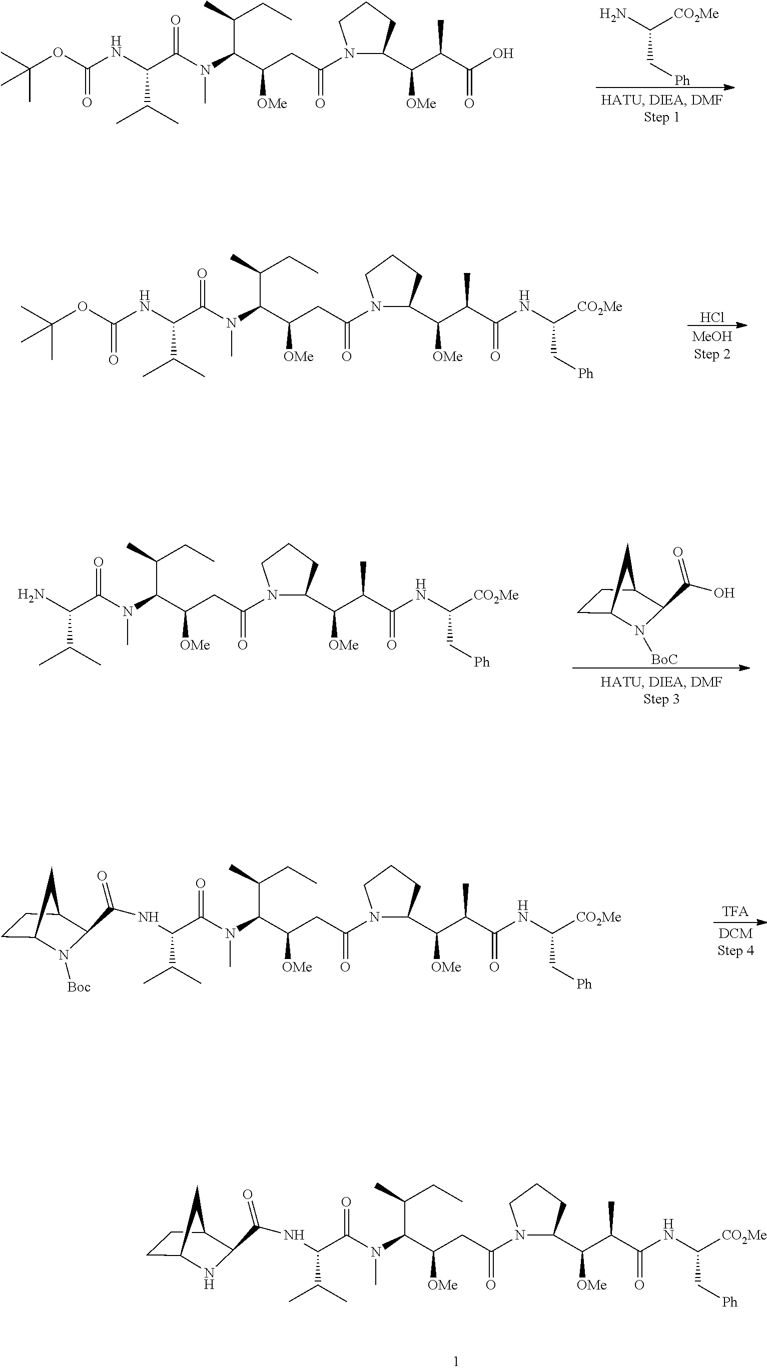

Step 1: To a solution of BocVal-Dil-Dap-OH (1.00 g, 1.75 mmol) in N,N-dimethylformamide (DMF, 20.0 mL) at 0° C. were added N,N-diisopropyl ethylamine (DIEA, 0.677 g, 5.25 mmol) and 1-[bis(dimethylamino)methylene]-1H-1,2,3-triazolo[4,5-b]pyridinium 3-oxid hexafluorophosphate (HATU) (0.731 g, 1.93 mmol). The resulting solution was then stirred for 5 minutes and added to a solution of L-phenylalanine methyl ester HCl salt (0.377 g, 1.75 mmol) and DIEA (0.226 g, 1.75 mmol) in DMF (5.0 mL) at 0° C. The reaction mixture was warmed to room temperature, stirred for an additional 30 minutes and then concentrated. The residue was purified by reverse phase HPLC using the ISCO system, C18 column, eluted with 20-90% acetonitrile-water to obtain BocVal-Dil-Dap-PheOMe: MS m/z 733.4 (M+1); retention time 1.47 minutes.

Step 2: To a solution of BocVal-Dil-Dap-PheOMe (0.683 g, 0.932 mmol) obtained in step 1 in methanol (20 mL) was added HCl (4N in 1,4-dioxane, 16 mL). The reaction mixture was stirred at room temperature for 7 hours and concentrated. The residue was dissolved in dioxane and lyophilized to obtain Val-Dil-Dap-PheOMe HCl salt: MS m/z 633.4 (M+1); retention time 0.96 minutes.

Step 3: (1R,3S,4S)—N-Boc-2-azabicyclo[2.2.1]heptane-3-carboxylic acid (12.6 mg, 0.052 mmol) was dissolved in DMF (1 mL) in a 15 ml round bottom flask. DIEA (12.3 mg, 0.095 mmol) and HATU (19 mg, 0.050 mmol) were added. The reaction mixture was stirred for 10 minutes and Val-Dil-Dap-PheOMe HCl salt (30 mg, 0.090 mmol) in DMF (1.0 mL) was added. The reaction mixture was stirred for 1 hour. LCMS analysis indicated the reaction was complete. The crude was purified by reverse phase HPLC using C18 column, eluted with 20-90% acetonitrile-H₂O containing 0.05% trifluoroacetic acid (TFA). The fractions containing the desired product were pooled and concentrated to obtain (1R,3S,4S)-tert-butyl 3-(((S)-1-(((3R,4S,5S)-3-methoxy-1-((S)-2-((1R,2R)-1-methoxy-3-(((S)-1-methoxy-1-oxo-3-phenylpropan-2-yl)amino)-2-methyl-3-oxopropyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate: MS m/z 856.6 (M+1); retention time 1.67 minutes.

Step 4: The product obtained in step 3 was dissolved in dichloromethane (DCM) (2.0 mL) and treated with TFA (0.5 mL). The reaction mixture was stirred at room temperature for 1 hour. LCMS analysis showed the reaction was complete. The reaction mixture was concentrated by rotary evaporator to give compound 1 as a TFA salt: MS m/z 756.6 (M+1); retention time 1.22 minutes.

### Example 3-2

### (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (2)

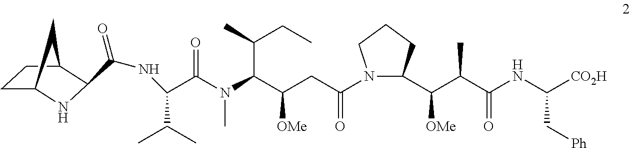 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@H]2CC[C@H]1C2)C(C)C]

In a 25 mL round bottom flask were added (S)-methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate TFA salt (1) (38.4 mg, 0.044 mmol), LiOHmonohydrate (50.0 mg, 1.19 mmol) and a solvent mixture of MeOH—H₂O (2:1, 4.0 mL). The mixture was stirred at room temperature for 60 hours. The LC-MS analysis indicated the reaction was complete. The reaction mixture was concentrated and purified by reverse phase HPLC, C18 column, eluted with acetonitrile-H₂O (10-70%) containing 0.05% TFA. The fractions containing the desired product were combined and concentrated to give compound 2 as a TFA salt, MS m/z 742.5 (M+1). Retention time 1.15 minutes.

### Example 3-3

### (1R,3S,4S)—N—((S)-1-(((3R,4S,5S)-1-((S)-2-((1R,2R)-3-(((1S,2R)-1-Hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (3)

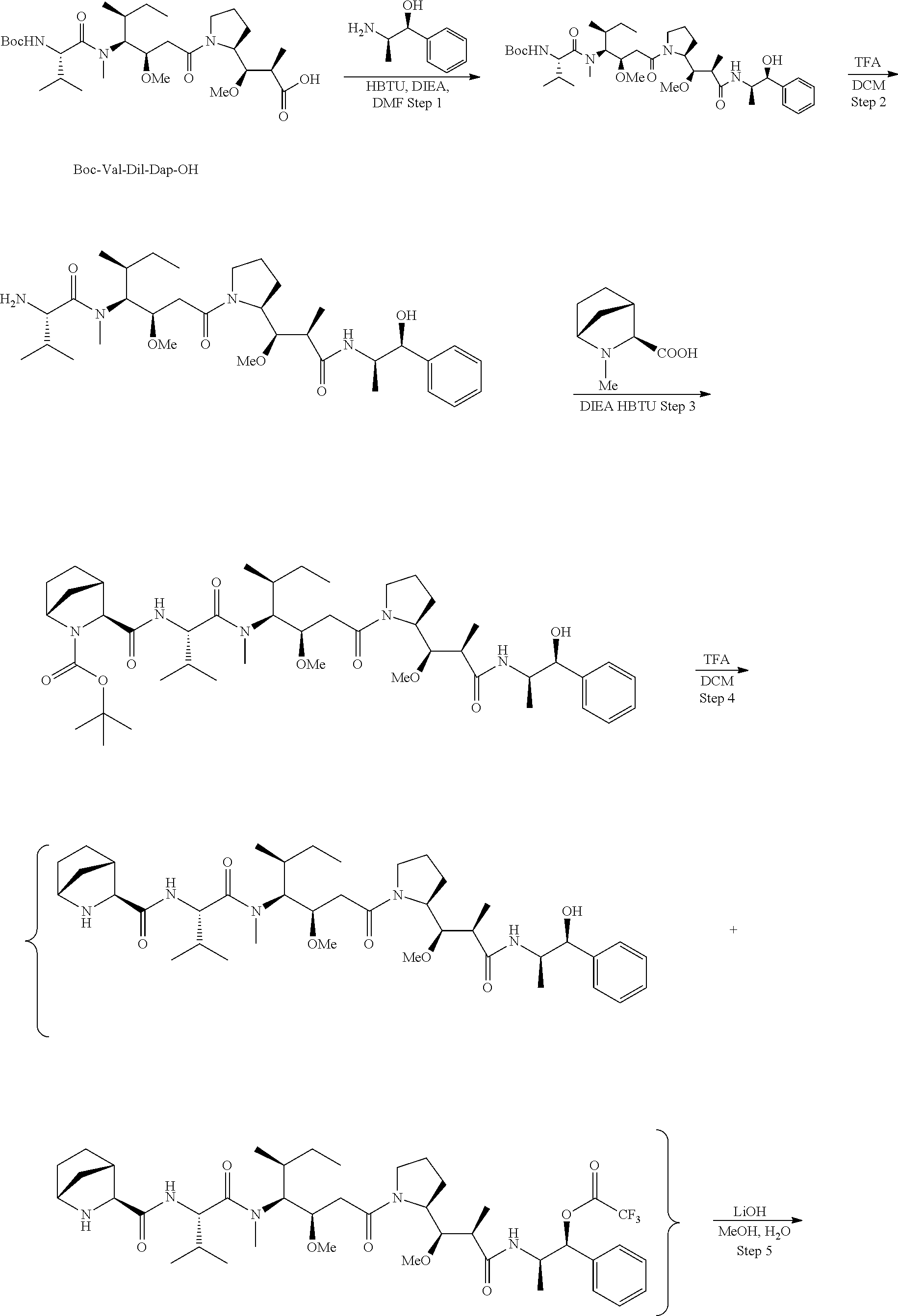

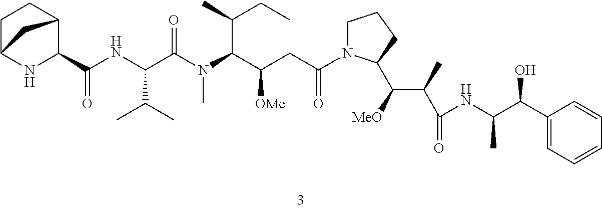 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H]1N[C@@H]2CC[C@H]1C2)C(C)C]

Step 1: To a solution of Boc-Val-Dil-Dap-OH (20.0 mg, 0.035 mmol) in DMF (1.0 mL) in a 15 mL round bottom flask was added DIEA (9.0 mg, 0.070 mmol), followed by N,N,N′,N′-tetramethyl-O-(1H-benzotriazol-1-yl)uronium hexafluorophosphate (HBTU) (13.3 mg, 0.035 mmol). The reaction mixture was stirred for 10 minutes before (1S,2R)-2-amino-1-phenylpropan-1-ol (6.4 mg, 0.042 mmol) in DMF (1.0 mL) was added to the reaction mixture. The reaction was stirred for 1 hour. LCMS analysis indicated the reaction was complete. The crude was purified by reverse phase HPLC, C18 column, eluted with 20-70% acetonitrile-H₂O containing 0.05% TFA. The fractions containing the desired product were pooled and concentrated to obtain tert-butyl ((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamate, MS m/z 705.4 (M+1). Retention time 1.39 minutes.

Step 2: To a solution of tert-butyl ((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamate (24.7 mg, 0.035 mmol) in DCM (2.0 mL) was added TFA (1.0 mL). The reaction mixture was stirred at room temperature for 2 hours and concentrated to obtain a mixture of (S)-2-amino-N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethylbutanamide (MS m/z 605.4 (M+1) Retention time 0.96 minutes and the TFA ester thereof (MS m/z 701.4 (M+1)), Retention time 1.17 minutes. The mixture was used in the next step without further purification.

Step 3: To a solution of (1R,3S,4S)-2-(tert-butoxycarbonyl)-2-azabicyclo[2.2.1]heptane-3-carboxylic acid (8.4 mg, 0.035 mmol) in DMF (1.0 mL) were added DIEA (0.024 ml, 0.14 mmol) and HBTU (13.3 mg, 0.035 mmol). The reaction mixture was stirred for 10 minutes and added to a solution of the product mixture obtained in step 2 (25.2 mg, 0.035 mmol) (containing TFA ester) in DMF (0.5 mL). The reaction mixture was kept at room temperature for 18 hours and then the crude was purified by reverse phase HPLC, C18 column, eluted with 30-90% acetonitrile-H₂O, containing 0.05% TFA. The fractions containing the desired products were concentrated to obtain a mixture of (1R,3S,4S)-tert-butyl 3-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (MS m/z 828.5 (M+1)) Retention time 1.42 minutes and the TFA ester thereof (MS m/z 924.4 (M+1)) Retention time 1.61 minutes.

Step 4: To a solution of the mixture obtained in step 3 in DCM (1.5 mL) was added TFA (1.0 mL). The reaction mixture was stirred at room temperature for 1 hour and then concentrated to obtain a mixture of (1R,3S,4S)—N—((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (MS m/z 728.4 (M+1)), retention time 0.99 minutes and (1S,2R)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-1-phenylpropyl 2,2,2-trifluoroacetate (MS m/z 824.5 (M+1)), retention time 1.31 minutes. This mixture was used in the next step without further purification.

Step 5: To a solution of the mixture obtained in step 4 in MeOH—H₂O (1:1, 3.0 mL) was added LiOH (10.0 mg, 0.418 mmol). The reaction mixture was stirred at room temperature for 18 hours and then concentrated to a total volume of approximately 1 mL. The crude mixture was purified by reverse phase HPLC, C18 column, eluted with 20-35% acetonitrile-H₂O, containing 0.05% TFA. The fractions containing the desired product were pooled and concentrated to obtain compound 3, MS m/z 728.4 (M+1). Retention time 0.99 minutes.

### Example 3-4

### (1R,3S,4S)—N—((S)-1-(((3R,4S,5S)-1-((S)-2-((1R,2R)-3-(((S)—N-1-(Methylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (4)

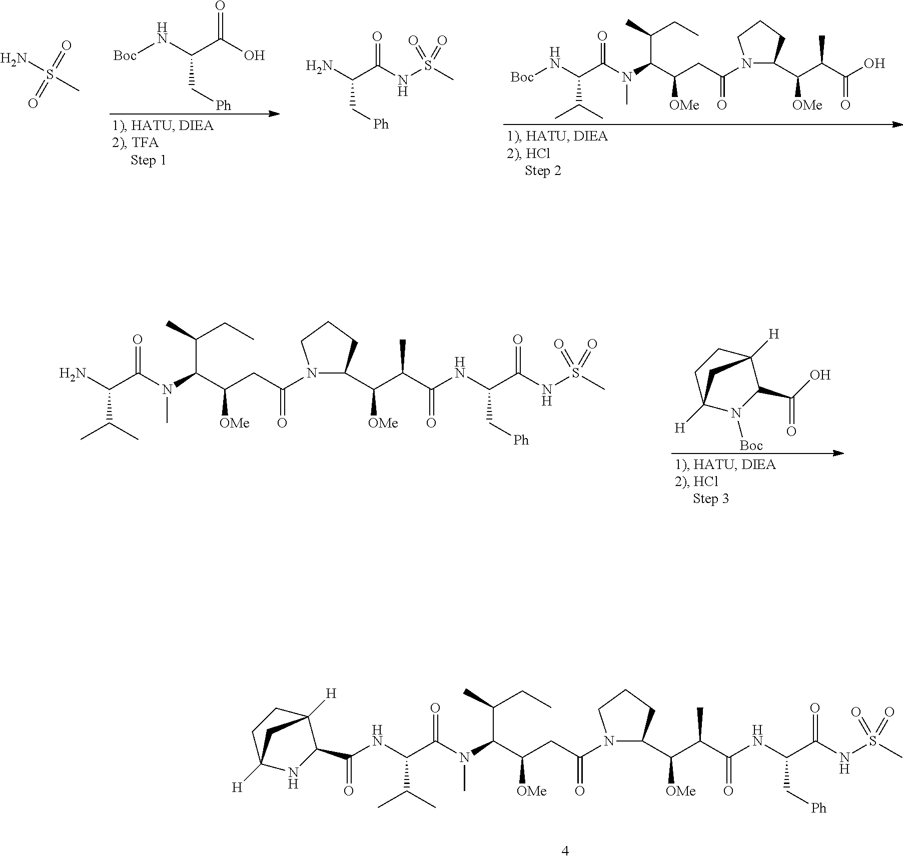

Step 1: (S)-2-((tert-butoxycarbonyl)amino)-3-phenylpropanoic acid (132.5 mg, 0.50 mmol) was dissolved in DMF (4 mL). DIEA (0.523 mL, 3.0 mmol) and HATU (475 mg, 1.25 mmol) were added. After 15 minutes, methanesulfonamide (143 mg) was added and The reaction mixture was stirred for 2 hours. LC/MS analysis indicated the completion of the reaction. The product was purified by Prep-HPLC, C18 column, eluted with 20-70% acetonitrile-H₂O containing 0.05% TFA. The fractions containing the desired product were pooled and lyophilized to obtain a white solid. MS m/z 243.1 (M+1). Retention time 1.023 minutes. The product was dissolved in DCM (2 mL). TFA (2 mL) was added and stirred for 1 hour at room temperature. LC/MS analysis indicated the reaction was completed. The deprotected product was purified by Prep-HPLC too, eluted with 10-40% acetonitrile-H₂O containing 0.05% TFA. The fractions containing the desired product were pooled and lyophilized to obtain a white solid. MS m/z 243.1 (M+1). Retention time 0.403 minutes. NMR (400 MHz, CD₃OD): δ 7.41-7.30 (m, 5H), 4.10-4.06 (m, 1H), 3.32-3.25 (m, 1H), 3.19 (s, 3H), 3.12-3.07 (m, 1H).

Step 2: Boc-Val-Dil-Dap (65.5 mg, 0.115 mmol) was dissolved in DMF (2 mL). DIEA (59.2 mg, 80 uL) and HATU (27.7 mg, 0.099 mmol) were added. After 10 minutes, (S)-2-amino-N-(methylsulfonyl)-3-phenylpropanamide (18.5 mg, 0.076 mmol) was added and The reaction mixture was stirred for 1 hour at room temperature. LC/MS analysis indicated the completion of the reaction. The product was purified by Prep-HPLC, C18 column, eluted with 10-90% acetonitrile-H₂O containing 0.05% TFA. The fractions containing the desired product were pooled and lyophilized to obtain a white solid. MS m/z 796.4 (M+1). Retention time 1.388 minutes. The product was dissolved in HCl in MeOH (3M, 3 mL). The solvent was removed slowly. LC/MS analysis indicated the completion of the reaction. MS m/z 696.3 (M+1). Retention time 1.046 minutes.

Step 3: (1R,3S,4S)-2-(tert-butoxycarbonyl)-2-azabicyclo[2.2.1]heptane-3-carboxylic acid (14.23 mg, 0.059 mmol) was dissolved in DMF (2 mL). DIEA (22.9 mg, 0.177 mmol) and HATU (20.19 mg, 0.053 mmol) were added. After 10 minutes, the product from the previous step (21.6 mg, 0.029 mmol) was added and The reaction mixture was stirred for 2 hours at room temperature. LC/MS analysis indicated the completion of the reaction. The product was purified by Prep-HPLC, C18 column, eluted with 10-90% acetonitrile-H₂O containing 0.05% TFA. The The fractions containing the desired product were pooled and lyophilized to obtain a white solid. MS m/z 919.5 (M+1). Retention time 1.370 minutes. The product was dissolved in HCl in MeOH (3M, 3 mL). The solvent was removed slowly. LC/MS analysis indicated the completion of the reaction. MS m/z 819.5 (M+1). Retention time 1.096 minutes.

### Example 3-5

### (1R,3S,4S)—N—((S)-1-(((3R,4S,5S)-1-((S)-2((1R,2R)-3-(((S)—N-1-(Methylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (5)

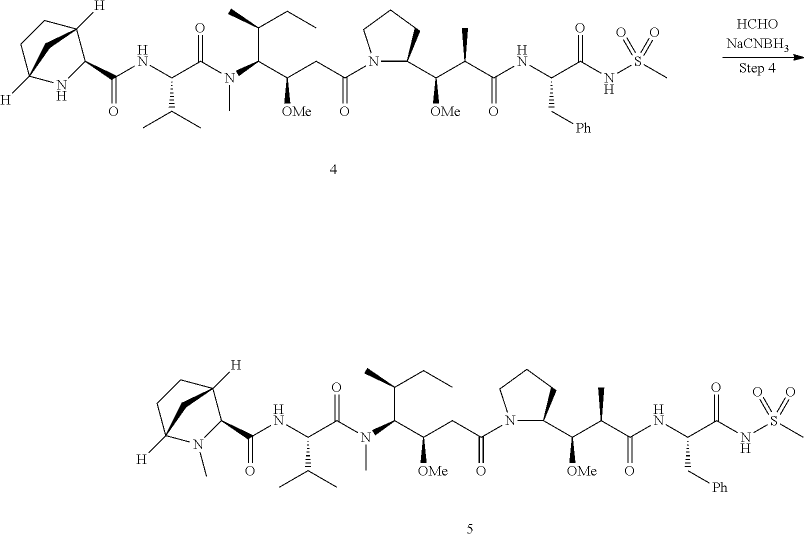 [SMILES: CC[C@H](C)[C@@H]([C@@H](CC(=O)N1CCC[C@H]1[C@H](OC)[C@@H](C)C(=O)N[C@@H](Cc1ccccc1)C(=O)NS(C)(=O)=O)OC)N(C)C(=O)[C@@H](NC(=O)[C@H]1N[C@@H]2CC[C@H]1C2)C(C)C ; CC[C@H](C)[C@@H]([C@@H](CC(=O)N1CCC[C@H]1[C@H](OC)[C@@H](C)C(=O)N[C@@H](Cc1ccccc1)C(=O)NS(C)(=O)=O)OC)N(C)C(=O)[C@@H](NC(=O)[C@@H]1[C@H]2CC[C@H](C2)N1C)C(C)C]

Compound 4 (5 mg, 0.00584 mmol) was dissolved in MeOH (2.0 mL). Paraformaldehyde (5.97 mg, 0.199 mmol) and acetic acid (6.0 uL) were added. Sodium cyanoborohydride (12.5 mg, 0.199 mmol) was added and the reaction mixture was heated to 50° C. and stirred for 1 hour. LC/MS analysis indicated the completion of the reaction. The product was purified by Prep-HPLC, C18 column, eluted with 10-90% acetonitrile-H₂O containing 0.05% TFA. The fractions containing the desired product were pooled and lyophilized to obtain a white solid. MS m/z 833.5 (M+1). Retention time 0.983 minutes.

### Example 3-6

### (1R,3S,4S)—N—((S)-1-(((3R,4S,5S)-3-methoxy-1((S)-2-((1R,2R)-1-Methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (6)

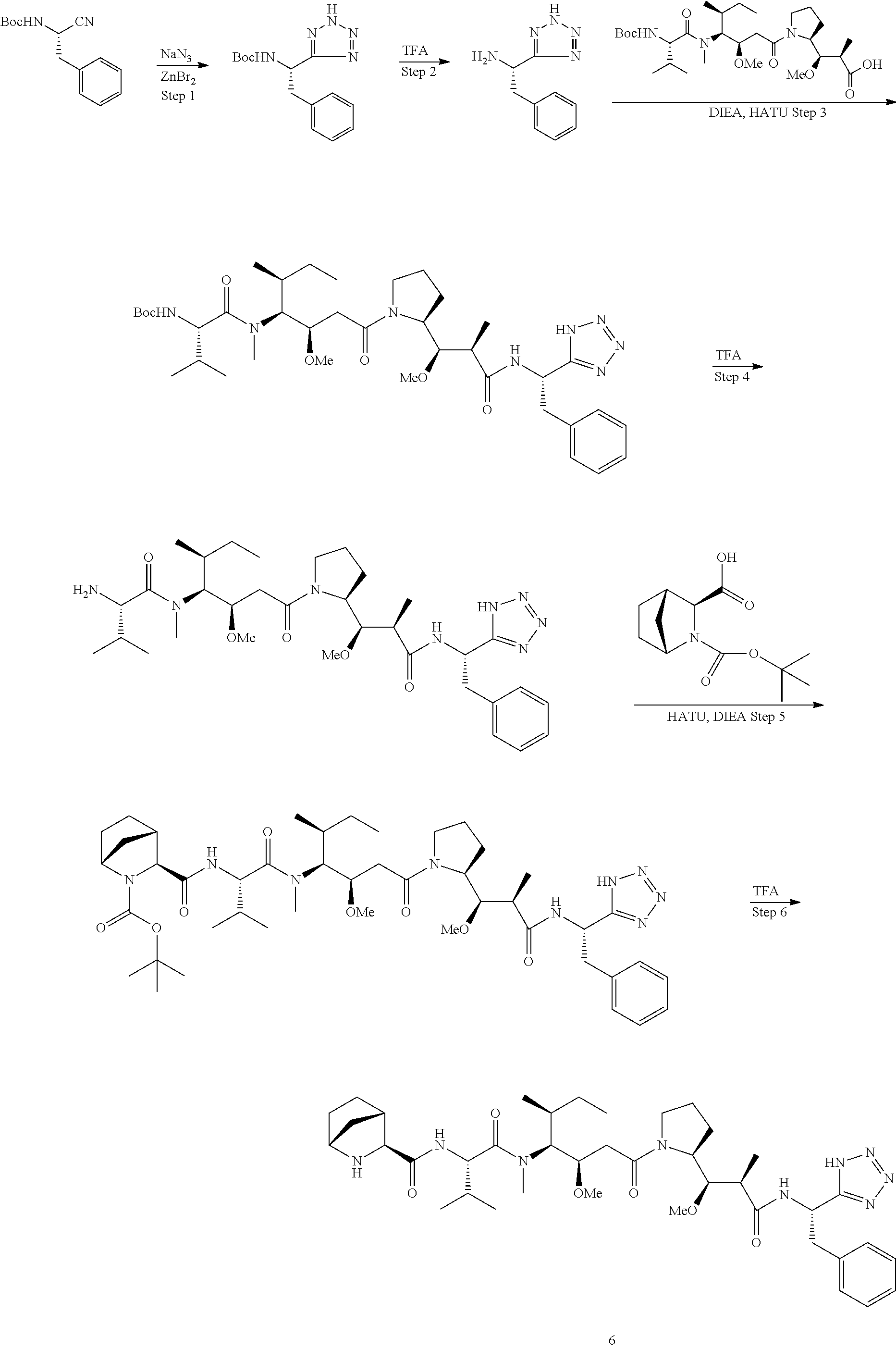

Step 1: N-Boc-amino nitrile (0.5 g, 2.03 mmol), sodium azide (0.264 g, 4.06 mmol) and zinc bromide (0.229 g, 1.02 mmol) were dissolved in a mixture of 2-propanol-water solvent mixture (1:1, 60 ml) and The reaction mixture was stirred at reflux for 16 hours. After completion of the reaction, 5 ml of 10% citric acid and 30 ml ethyl acetate were added and stirring was continued until no solid remained. The aqueous layer was extracted twice with ethyl acetate. The combined organic layer was washed with water and dried over anhydrous Na₂SO₄. The solvent was removed and the residue was purified by silica gel column, eluted with 10% methanol in DCM. Fractions containing the desired product were concentrated, re-dissolved in ethyl acetate, washed with brine, dried and concentrated to give (S)-tert-butyl (2-phenyl-1-(2H-tetrazol-5-yl)ethyl)carbamate MS m/z 290.2 (M+1). ¹H NMR (400 MHz, CDCl₃) δ 7.40-7.24 (m, 3H), 7.22-7.12 (m, 2H), 5.22-5.02 (m, 2H), 3.49-3.24 (m, 2H), 1.40 (s, 9H).

Step 2: In a 15 ml round bottom flask was added (S)-tert-butyl (2-phenyl-1-(2H-tetrazol-5-yl)ethyl)carbamate (30 mg, 0.104 mmol), TFA (2 ml) and DCM (4 ml) to give a clear solution which was stirred at room temperature for 1 hour. LCMS showed the Boc group was cleaved. The solution was concentrated to obtain crude (S)-2-phenyl-1-(2H-tetrazol-5-yl)ethanamine as TFA salt (M+1 190.2), which was used without further purification in the next step.

Step 3: In a 15 ml round bottom flask was added Boc-Val-Dil-Dap-OH (59.3 mg, 0.104 mmol) and DIEA (0.072 ml, 0.415 mmol) in DMF (2 ml) give a clear solution. HATU (43.4 mg, 0.114 mmol) was added and the reaction mixture was then stirred for 5 minutes and then (S)-2-phenyl-1-(2H-tetrazol-5-yl)ethanamine TFA salt obtained in step 2 (0.104 mmol) was added. The solution was stirred at room temperature for 72 hours. The crude was purified by reverse phase HPLC, C18 column, eluted with 10-70% acetonitrile-H₂O, containing 0.05% TFA. Fractions containing desired product were concentrated to obtain tert-butyl ((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)carbamate MS m/z 743.5 (M+1). Retention time 1.325 minutes.

Step 4: In a 15 ml round bottom flask was added tert-butyl ((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)carbamate (46 mg, 0.056 mmol), TFA (2 ml) and DCM (4 ml) to give a clear solution which was stirred at room temperature for 1 hour. LCMS showed the Boc group was cleaved. The solution was concentrated to obtain crude (S)-2-amino-N-((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)-N,3-dimethylbutanamide TFA salt. MS m/z 643.5 (M+1). Retention time 0.947 minutes, which was used in the next step without further purification.

Step 5: To a solution of (1R,3S,4S)-2-(tert-butoxycarbonyl)-2-azabicyclo[2.2.1]heptane-3-carboxylic acid (7.6 mg, 0.032 mmol) in DMF (1 ml) was added DIEA (0.014 ml, 0.079 mmol) and HATU (12 mg, 0.032 mmol), which was then added to a solution of (S)-2-amino-N-((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)-N,3-dimethylbutanamide TFA salt (20 mg, 0.026 mmol). The reaction mixture was stirred at room temperature for 2 hours and then the crude was purified by reverse phase HPLC, C18 column, eluted with 30-70% acetonitrile-H₂O, containing 0.05% TFA. The fractions containing desired product were concentrated to obtain (1R,3S,4S)-tert-butyl 3-(((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate as TFA salt MS m/z 866.6 (M+1). Retention time 1.407 minutes.

Step 6: To a solution of (1R,3S,4S)-tert-butyl 3-(((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate TFA salt (10.2 mg, 0.012 mmol) in DCM (2 ml) was added TFA (1 ml). The reaction mixture was stirred at room temperature for 1 hour and then concentrated to obtain (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (6) as TFA salt. MS m/z 766.6 (M+1). Retention time 0.985 minutes.

### Example 3-7

### ((R)-1-((2R,3R)-3-((S)-1-((3R,4S,5S)-4-((S)-2-((1R,3S,4S)-2-Azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid. (7)

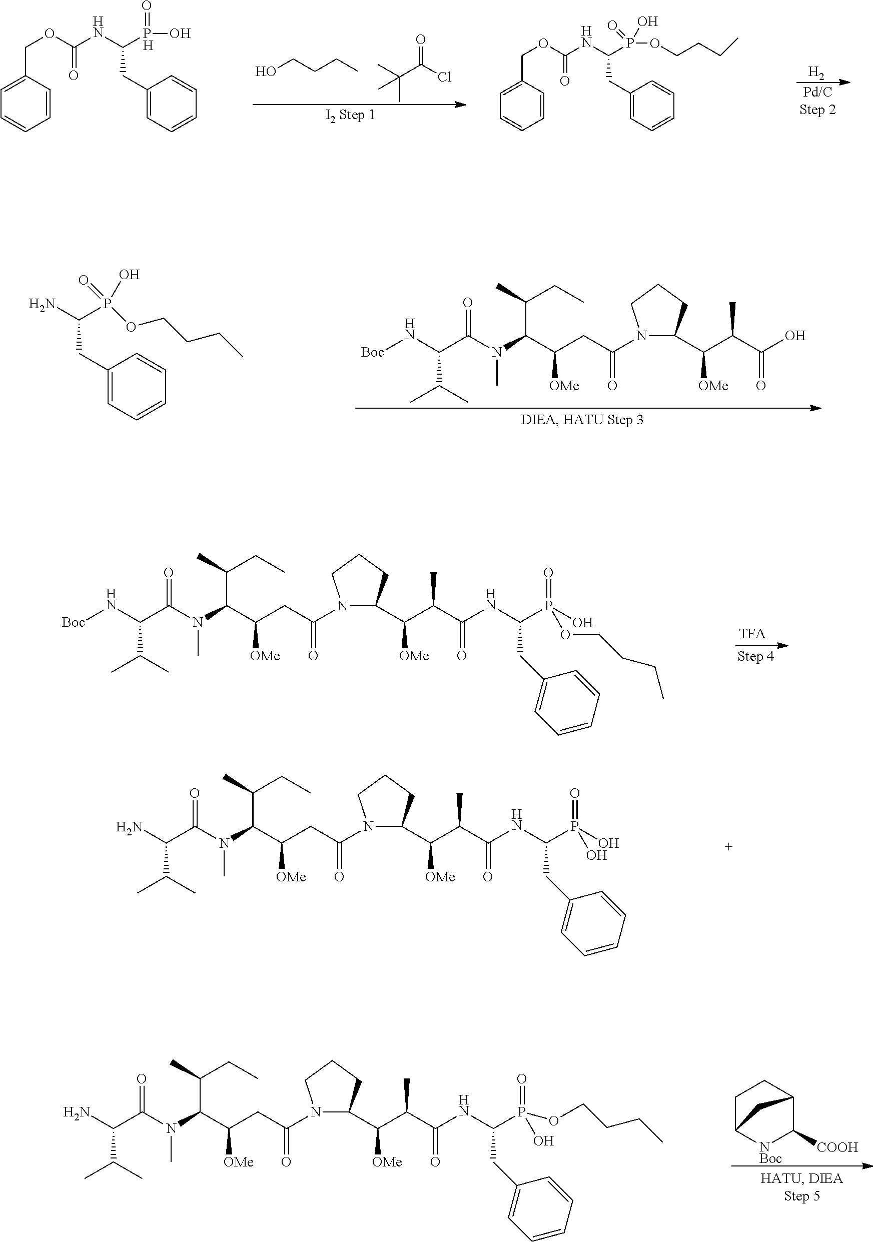

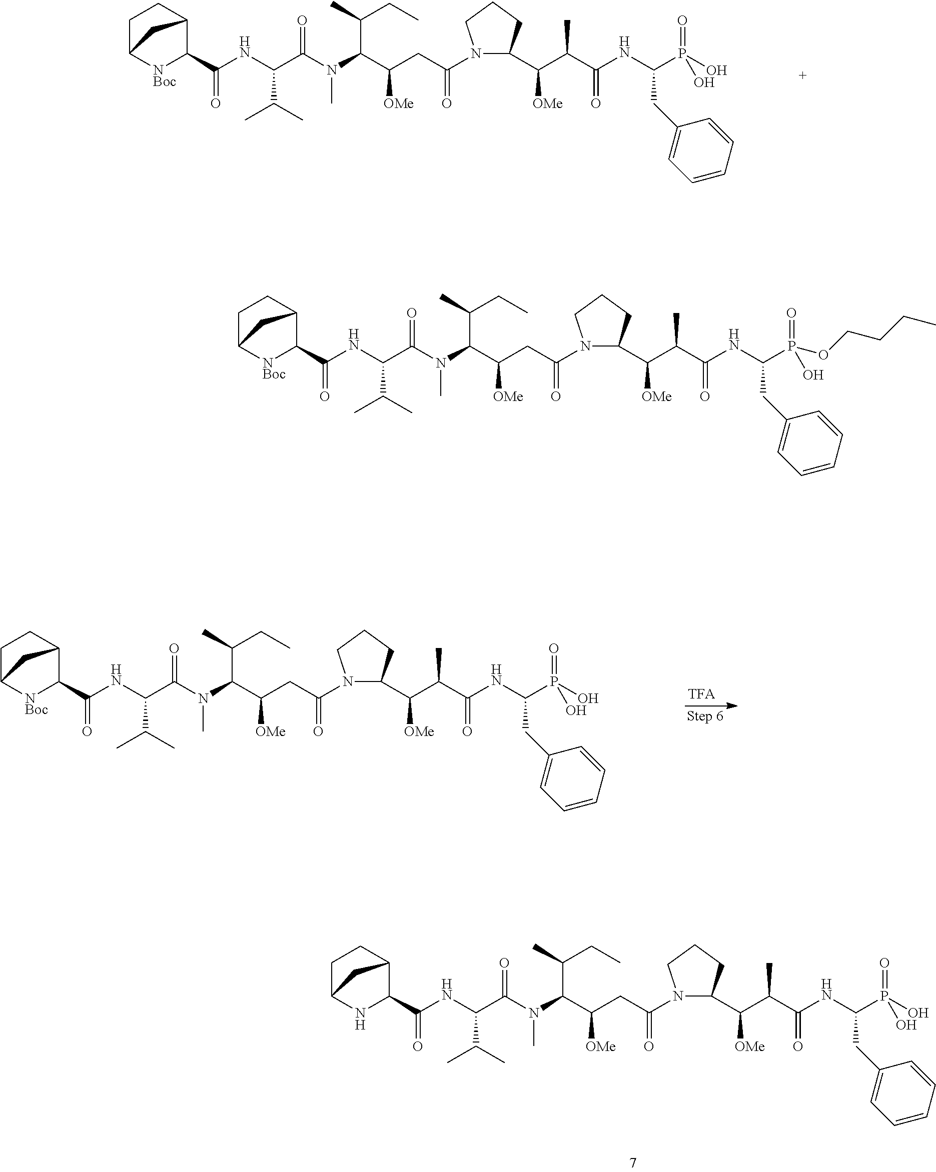

Step 1: ((R)-1-(((Benzyloxy)carbonyl)amino)-2-phenylethyl)phosphinic acid (100 mg, 0.313 mmol), (synthesized by following the procedure described in J. Chem. Soc. Perkin Trans. I 1984, 2845) was dissolved in pyridine (5 ml) and n-BuOH (35 mg, 0.46 mmol) was added, followed by pivaloyl chloride (70 mg, 0.58 mmol). LCMS indicated the reaction was incomplete, therefor three other portions of n-BuOH and pivaloyl chloride were added until all of the phosphinic acid was consumed. Then a solution of iodine (160 mg, 0.630 mmol) in 2 ml pyridine-H₂O (10% water) was added and the reaction misture was stirred for 20 minutes. LCMS indicated that the reaction was complete. Pyridine was removed by vacuum. Thiosulfate aqueous solution was added and the reaction mixture was extracted with EtOAc. EtOAc layer was then dried, concentrated and purified with ISCO (5.5 g C18 column), eluted with 10%-60% acetonitrile in water with 0.5% TFA to obtain benzyl ((1R)-1-(butoxy(hydroxy)phosphoryl)-2-phenylethyl)carbamate as white solid. MS m/z 392.1 (M+1). Retention time 1.179 minutes. 1H NMR (400 MHz, CD₃CN) d 7.42-7.18 (m, 8H), 7.18-7.00 (m, 2H), 6.10 (s, 1H), 5.07-4.59 (m, 2H), 4.20-4.35 (m, 1H), 4.13-3.93 (m, 2H), 3.15-3.30 (m, 1H), 2.85-2.75 (s, 1H), 1.71-1.47 (m, 2H), 1.47-1.23 (m, 2H), 0.89 (t, J=7.3 Hz, 3H).

Step 2: To a solution of benzyl ((1R)-1-(butoxy(hydroxy)phosphoryl)-2-phenylethyl)carbamate (84.7 mg, 0.216 mmol) in MeOH (5 ml) were added 10% Pd/C (26 mg). A hydrogen balloon was attached and the reaction mixture was stirred at room temperature for 2 hours. The catalyst was removed by filtration through Celite, and the filtrates were evaporated to dryness to give butyl hydrogen ((R)-1-amino-2-phenylethyl)phosphonate. MS m/z 258.1 (M+1). Retention time 0.789 minutes, which was used in the next step without purification.

Step 3: In a 15 mL round-bottomed flask was added Boc-Val-Dip-Dap-OH (80 mg, 0.140 mmol) and DIEA (62.9 mg, 0.487 mmol) in DMF (2 ml) to give a clear solution. HATU (53 mg, 0.139 mmol) was added and the reaction mixture was stirred for 5 minutes and then butyl hydrogen ((R)-1-amino-2-phenylethyl)phosphonate (41.9 mg, 0.163 mmol) was added. The solution was stirred at room temperature for 18 hours. The crude was purified by reverse phase HPLC, C18 column, eluted with 40-60% acetonitrile-H₂O, containing 0.05% TFA. The fractions containing desired product were concentrated to tert-butyl ((2S)-1-(((3R,4S,5S)-1-((2S)-2-((1R,2R)-3-(((1R)-1-(butoxy(hydroxy)phosphoryl)-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamate. MS m/z 811.4 (M+1). Retention time 1.376 minutes.

Step 4: To a solution tert-butyl ((2S)-1-(((3R,4S,5S)-1-((2S)-2-((1R,2R)-3-(((1R)-1-(butoxy(hydroxy)phosphoryl)-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamate (106 mg, 0.131 mmol) in DCM (3 ml) was added TFA (1 ml), and the reaction mixture was stirred at room temperature for 1 hour and then concentrated. About ⅔ converted to phosphonic acid (1-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-2-phenylethyl)phosphonic acid. MS m/z 655.3 (M+1). Retention time 0.957 minutes. The other ⅓ was butyl hydrogen (1-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-2-phenylethyl)phosphonate. MS m/z 711.4 (M+1). Retention time 1.038 minutes. The mixture was used in the next step without separation.

Step 5: To a solution of (1R,3S,4S)-2-(tert-butoxycarbonyl)-2-azabicyclo[2.2.1]heptane-3-carboxylic acid (3.8 mg, 0.016 mmol) in DMF (1 ml) was added DIEA (6.1 mg, 0.047 mmol) and then HATU (5.9 mg, 0.016 mmol). The reaction mixture was stirred at room temperature for 10 minutes and then added to a mixture of the amine from step 4 (12 mg, 0.016 mmol) containing mainly the phosphonic acid. The reaction mixture was stirred at room temperature for 1 hour. The crude was purified by ISCO using C18 column, 4.5 g, eluted with 10-70% acetonitrile in water with 0.05% TFA. The fractions containing desired product were concentrated to obtain ((R)-1-((2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid MS m/z 878.5 (M+1). Retention time 1.307 minutes, and (1R,3S,4S)-tert-butyl 3-(((2S)-1-(((3R,4S,5S)-1-((2S)-2-((1R,2R)-3-(((1R)-1-(butoxy(hydroxy)phosphoryl)-2-phenylethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate MS m/z 934.5 (M+1). Retention time 1.447 minutes.

Step 6: To a solution of ((R)-1-((2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid (11.0 mg, 0.012 mmol) in DCM (2 ml) was added TFA (1 ml). The reaction mixture was stirred at room temperature for 1 hour and then concentrated to obtain ((R)-1-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid (7). MS m/z 778.4 (M+1). Retention time 0.973 minutes.

### Example 3-8

### ((R)-1-((2R,3R)-3-((S)-1-((3R,4S,5S)-4-((S)-2-((1R,3S,4S)-2-Azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid (8)

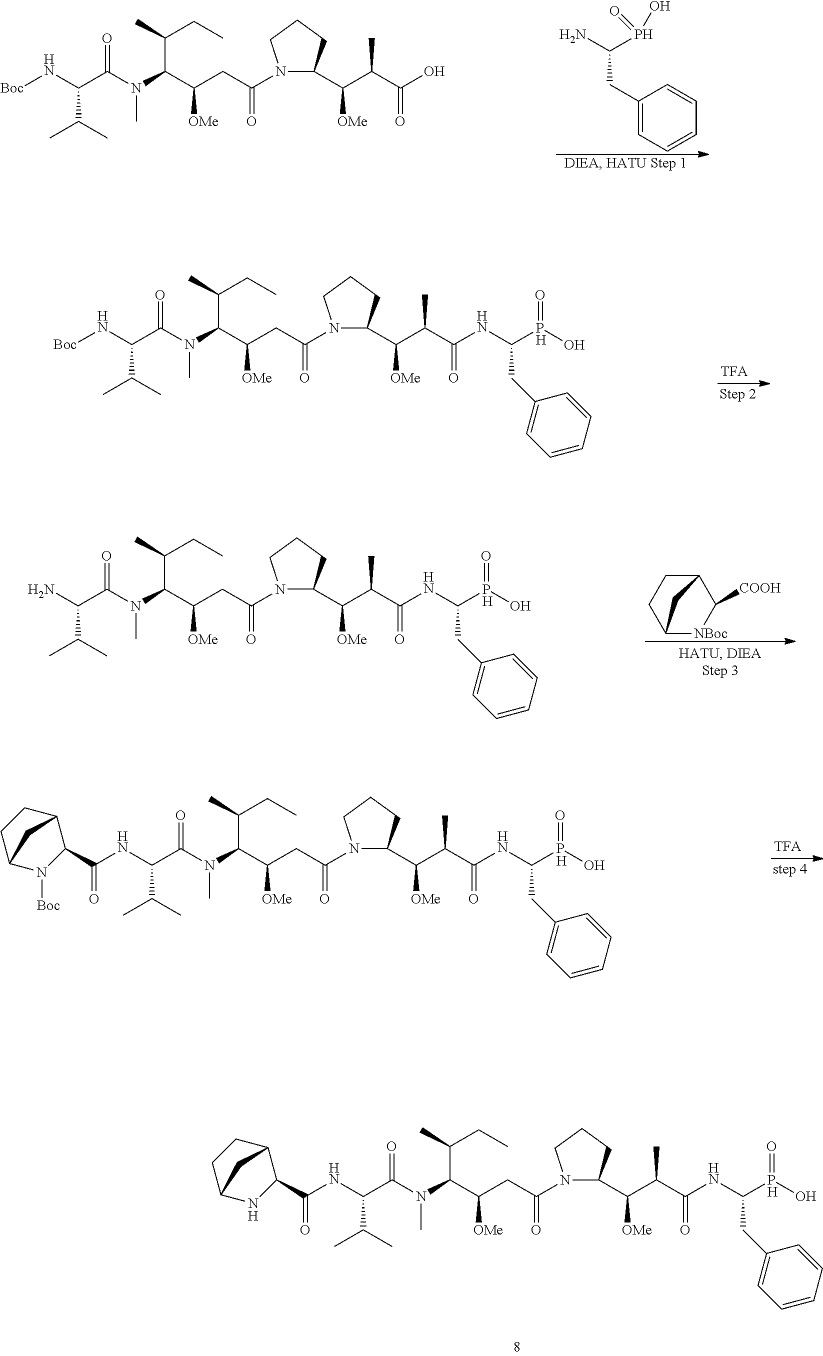

Step 1: In a 15 mL round-bottomed flask was added Boc-Val-Dip-Dap-OH (50 mg, 0.087 mmol) and DIEA (33.9 mg, 0.262 mmol) in DMF (2 mL) to give a clear solution. HATU (33.3 mg, 0.087 mmol) was added and the reaction mixture was stirred for 5 minutes and then added to ((R)-1-amino-2-phenylethyl)phosphinic acid (41 mg, 0.154 mmol), (synthesized by following the procedure described in J. Chem. Soc. Perkin Trans. I 1984, 2845). The solution was stirred at room temperature for 18 hours. LCMS indicated the formation of the desired product. The crude was purified by reverse phase HPLC, C18 column, eluted with 30-50% acetonitrile-H₂O, containing 0.05% TFA. The fractions containing desired product were concentrated to obtain ((R)-1-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-2-phenylethyl)phosphinic acid. MS m/z 739.4 (M+1). Retention time 1.248 minutes.

Step 2: To a solution of ((R)-1-((2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanamido)-2-phenylethyl)phosphinic acid (69.1 mg, 0.094 mmol) in DCM (2 ml) was added TFA (1 ml) and the reaction mixture was stirred at room temperature for 1 hour and then concentrated to give ((R)-1-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-2-phenylethyl)phosphinic acid (MS m/z 639.3 (M+1); retention time 0.851 minutes) which was used without further purification in the next step.

Step 3: To a solution of (1R,3S,4S)-2-(tert-butoxycarbonyl)-2-azabicyclo[2.2.1]heptane-3-carboxylic acid (11.3 mg, 0.047 mmol) in DMF (1 ml) was added DIEA (0.033 ml, 0.188 mmol), followed by HATU (17.9 mg, 0.047 mmol). The reaction mixture was stirred for 10 minutes and then added to a solution of ((R)-1-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-2-phenylethyl)phosphinic acid (35.4 mg, 0.047 mmol) in DMF (1 ml). LCMS indicated the reaction was complete in 10 minutes. The crude was purified by reverse phase HPLC, C18 column, eluted with 30-55% acetonitrile-H₂O, containing 0.05% TFA. The fractions containing desired product were concentrated to obtain ((R)-1-((2R,3R)-3-((S)-1-((3R,4S,5S)-4-((S)-2-((1R,3S,4S)-2-(tert-butoxycarbonyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid. MS m/z 862.5 (M+1). Retention time 1.372 minutes.

Step 4: To a solution of ((R)-1-((2R,3R)-3-((S)-1-((3R,4S,5S)-4-((S)-2-((1R,3S,4S)-2-(tert-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid (60 mg, 0.070 mmol) in DCM (2 ml) was added TFA (1 ml). The reaction mixture was stirred at room temperature for 1 hour and then concentrated to give ((R)-1-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid (8). MS m/z 762.5 (M+1). Retention time 1.220 minutes.

### Example 3-9

### ((R)-1-((2R,3R)-3-((S)-1-((3R,4S,5S)-4-((S)—N,3-Dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-phenylethyl)phosphinic acid (9)

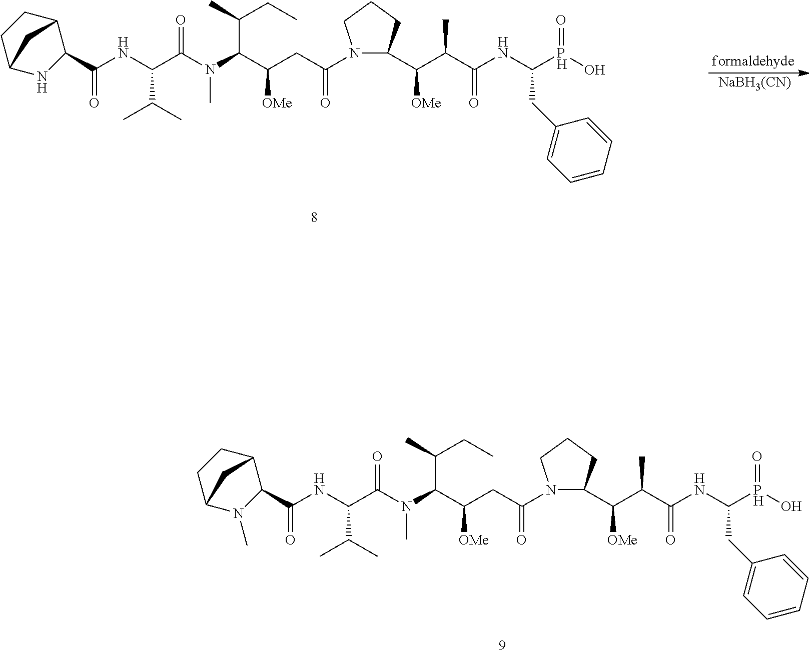

To a solution of ((R)-1-((2R,3R)-3-((S)-1-((3R,4S,5S)-4-((S)-2-((1R,3S,4S)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid (8) (20 mg, 0.023 mmol) in MeOH (2 ml) was added paraformaldehyde (10 mg, 0.33 mmol) and acetic acid (0.019 ml, 0.333 mmol), followed by sodium cyanoborohydride (20 mg, 0.32 mmol). The reaction mixture was stirred at 50° C. for 1 hour and then at room temperature for 2 days. LCMS indicated that the reaction was complete. The reaction mixture was filtered through Celite to remove the insoluble residue and the crude was purified by reverse phase HPLC, C18 column, eluted with 10-50% acetonitrile-H₂O, containing 0.05% TFA. The fractions containing desired product were concentrated to obtain ((R)-1-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-phenylethyl)phosphinic acid (9). MS m/z 776.4 (M+1). Retention time 0.944 minutes.

### Example 3-10

### (1R,3S,4S)—N—((S)-1-(((3R,4S,5S)-1-((S)-2-((1R,2R)-3-(((S)-1-Hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (50)

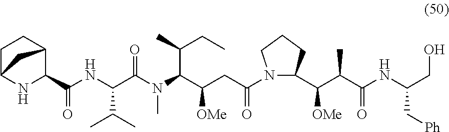 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@H]1N[C@@H]2CC[C@H]1C2)C(C)C]

Step 1: DIEA (0.013 ml, 0.075 mmol) and HATU (18.5 mg, 0.049 mmol) were added to (1R,3S,4S)-2-(tert-butoxycarbonyl)-2-azabicyclo[2.2.1]heptane-3-carboxylic acid (9.8 mg, 0.040 mmol) in DMF (1 ml). The reaction mixture was stirred for 5 min and then added to Val-Dil-Dap-OH (17.7 mg, 0.038 mmol) in DMF. The reaction was stirred at rt for 16 h. Then the crude was purified by preparative HPLC (30-70% acetonitrile-H₂O containing 0.05% TFA) to obtain (2R,3R)-3-((S)-1-((3R,4S,5S)-4-((S)-2-((1R,3S,4S)-2-(t-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid. MS m/z 695.4 (M+H). Retention time 1.376 min.

Step 2: To the product obtained in step 1 (5.9 mg, 0.008 mmol) in DMF (1 ml) were added DIEA (1.1 mg, 0.008 mmol) and HATU (3.8 mg, 0.010 mmol). After the reaction was stirred for 5 min, (S)-2-(S)-2-amino-3-phenylpropan-1-ol (1.9 mg, 0.013 mmol) in DMF was added. The reaction was stirred at rt for 1 h. The crude was purified by preparative HPLC (20-90% acetonitrile-H₂O containing 0.05% TFA) to obtain (1R,3S,4S)-t-butyl 3-(((S)-1-(((3R,4S,5S)-1-((S)-2-((1R,2R)-3-(((S)-1-hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate. MS m/z 828.5 (M+H). Retention time 1.388 min.

Step 3: The product obtained in step 2 (4 mg, 0.005 mmol) in DCM (3 ml) was treated with TFA (1 ml) at rt for 1 h and then concentrated to give compound (50) as TFA salt. MS m/z 728.5 (M+H). Retention time 1.008 min.

### Example 3-11

### (1R,3S,4S)—N—((S)-1-(((3R,4S,5S)-1-((S)-2-((1R,2R)-3-(((S)-1-Hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (51)

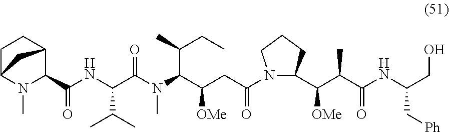 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@H]2CC[C@H](C2)N1C)C(C)C]

(1R,3S,4S)—N—((S)-1-(((3R,4S,5S)-1-((S)-2-((1R,2R)-3-(((S)-1-Hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (50) (6.1 mg, 0.008 mmol), MeOH (2 ml), acetic acid (0.005 ml, 0.09 mmol), paraformaldehyde (3 mg, 0.1 mmol), and sodium cyanoborohydride (5 mg, 0.08 mmol) were combined at rt and then stirred at 50° C. for 1 h. The reaction mixture was then cooled to rt, filtered, and purified by preparative HPLC (20-40% acetonitrile-H₂O containing 0.05% TFA) to obtain compound (51) as TFA salt. MS m/z 742.5 (M+H). Retention time 1.008 min.

### Example 3-12

### (1R,3S,4S)—N—((S)-1-(((3R,4S,5S)-1-((S)-2-((1R,2R)-3-(((S)-1-(3-Aminophenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (52)

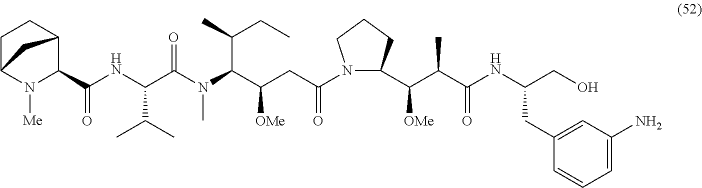 [SMILES: CC[C@H](C)[C@@H]([C@@H](CC(=O)N1CCC[C@H]1[C@H](OC)[C@@H](C)C(=O)N[C@H](CO)Cc1cccc(N)c1)OC)N(C)C(=O)[C@@H](NC(=O)[C@@H]1[C@H]2CC[C@H](C2)N1C)C(C)C]

Step 1: DIEA (0.105 ml, 0.60 mmol) and HATU (45.5 mg, 0.12 mmol) were added to (3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoic acid (i-7) (57 mg, 0.12 mmol) in DMF (2 ml). The reaction mixture was stirred at rt for 5 min and then DapOMe (i-9) (28.5 mg, 0.12 mmol) in DMF (1 ml) was added. The reaction mixture was stirred at rt for 1 h and then purified by preparative HPLC (10-50% acetonitrile-H₂O containing 0.05% TFA) to obtain (2R,3R)-methyl 3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate. MS m/z 623.5 (M+H). Retention time 1.225 min.

Step 2: LiOH (30 mg, 1.25 mmol) was added to the product obtained in step 1 (43.2 mg, 0.059 mmol) in MeOH—H₂O (1:1, 4 ml). The reaction mixture was stirred at rt for 18 h, concentrated and acidified with HCl (1N, 1 ml). The crude was purified by preparative HPLC (10-38% acetonitrile-H₂O containing 0.05% TFA) obtain (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid as TFA salt. MS m/z 609.5 (M+H). Retention time 0.962 min.

Step 3: To the product obtained in step 2 (45.7 mg, 0.063 mmol) in DMF (1 ml) were added DIEA (0.055 ml, 0.32 mmol) and HATU (24.0 mg, 0.063 mmol). The reaction mixture was stirred at rt for 10 min and then added to (S)-t-butyl (3-(2-amino-3-hydroxypropyl)phenyl)carbamate TFA salt (i-4) (24.1 mg, 0.063 mmol) in DMF (1 ml). The reaction mixture was stirred at rt for 1 h and then concentrated. The crude was purified by preparative HPLC (20-70% acetonitrile-H₂O containing 0.05% TFA) to obtain t-butyl (3-((S)-2-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)carbamate as TFA salt. MS m/z 857.5 (M+H). Retention time 1.145 min.

Step 4: A solution of the product obtained in step 3 (61.4 mg, 0.063 mmol) in acetonitrile-water (1:1, 4 ml) with 5% HCl was stirred at rt for 24 h. The reaction mixture was then concentrated and purified by preparative HPLC (10-30% acetonitrile-H₂O containing 0.05% TFA) to give compound (52) as TFA salt. MS m/z 757.5 (M+H). Retention time 0.744 min.

### Example 3-13

### (S)-Methyl 2-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoate (53)

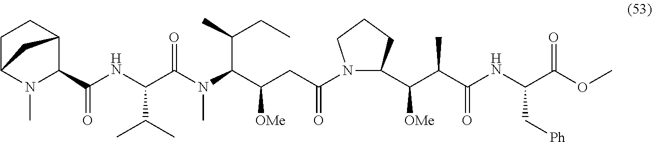 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@@H]1[C@H]2CC[C@H](C2)N1C)C(C)C]

To (S)-methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate TFA salt (1) (55.4 mg, 0.064 mmol) in MeOH (5 ml) were added acetic acid (0.009 ml, 0.2 mmol), paraformaldehyde (24 mg, 0.79 mmol) and then sodium cyanoborohydride (25 mg, 0.40 mmol). The reaction mixture was stirred at 40° C. for 16 h, filtered, concentrated and purified by preparative HPLC (10-45% acetonitrile-water with 0.05% TFA) to compound (53) as TFA salt. MS m/z 770.3 (M+H). Retention time 1.100 min.

### Example 3-14

### (S)-2-((2R,3R)-3-((S)-1-((3R,4S,5S)-4-((S)—N,3-Dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoic acid (9d)

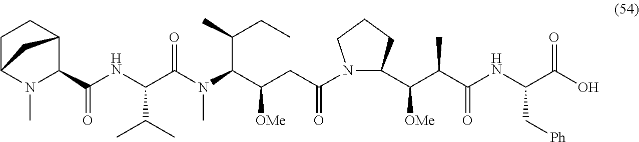 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C)C(C)C]

Compound (53) TFA salt (50.8 mg, 0.057 mmol) was dissolved in MeOH—H₂O (1:1, 5 ml) and LiOH (20 mg, 0.835 mmol) was added. The reaction was stirred at 40° C. for 1 h. MeOH was removed by evaporation. Water was added to the residue, and acidified with AcOH (0.040 ml). The crude was purified by preparative HPLC (27-33% acetonitrile-H₂O containing 0.05% TFA) to obtain compound (54) as TFA salt. MS m/z 756.5 (M+H). Retention time 0.985 min.

### Example 3-15

### (1R,3S,4S)—N—((S)-1-(((3R,4S,5S)-3-methoxy-1-((S)-2-((1R,2R)-1-Methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (55)

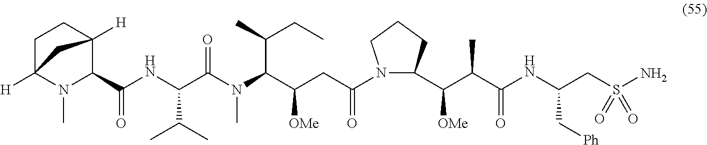 [SMILES: CC[C@H](C)[C@@H]([C@@H](CC(=O)N1CCC[C@H]1[C@H](OC)[C@@H](C)C(=O)N[C@@H](Cc1ccccc1)CS(N)(=O)=O)OC)N(C)C(=O)[C@@H](NC(=O)[C@@H]1[C@H]2CC[C@H](C2)N1C)C(C)C]

DIEA (10.2 mg, 0.014 ml) and HATU (7.7 mg, 0.020 mmol) were added to (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid TFA salt (Step 2, Example 3-12) (12.3 mg, 0.017 mmol) in DMF (1 ml). The reaction was stirred for 15 min, and then (S)-2-amino-3-phenylpropane-1-sulfonamide (4.3 mg, 0.020 mmol) in DMF (0.5 ml) was added. The reaction was stirred at rt for an additional 1 h. The crude was purified by preparative HPLC (20-70% acetonitrile-H₂O containing 0.05% TFA to obtain compound (55). MS m/z 805.5 (M+1). Retention time 0.965 min.

### Example 3-16

### (1R,3S,4S)—N—((S)-1-(((3R,4S,5S)-3-methoxy-1-((S)-2-((1R,2R)-1-Methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (56)

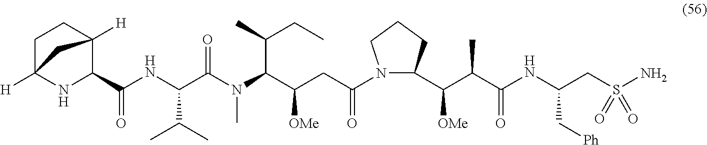 [SMILES: CC[C@H](C)[C@@H]([C@@H](CC(=O)N1CCC[C@H]1[C@H](OC)[C@@H](C)C(=O)N[C@@H](Cc1ccccc1)CS(N)(=O)=O)OC)N(C)C(=O)[C@@H](NC(=O)[C@H]1N[C@@H]2CC[C@H]1C2)C(C)C]

Step 1: To Boc-Dap-OH (21.6 mg, 0.075 mmol) in DMF (2 ml) were added DIEA (48.5 mg, 0.066 ml) and HATU (26.2 mg, 0.069 mmol). The reaction was stirred for 15 min, and then (S)-2-amino-3-phenylpropane-1-sulfonamide (13.4 mg, 0.063 mmol) was added. The reaction mixture was stirred at rt for 2 h and then purified by preparative HPLC (20-70% acetonitrile-H₂O containing 0.05% TFA) to obtain (S)-t-butyl 2-((1R,2R)-1-methoxy-2-methyl-3-oxo-3-(((S)-1-phenyl-3-sulfamoylpropan-2-yl)amino)propyl)pyrrolidine-1-carboxylate. MS m/z 484.2 (M+1). Retention time 1.130 min.

Step 2: (S)-t-Butyl 2-((1R,2R)-1-methoxy-2-methyl-3-oxo-3-(((S)-1-phenyl-3-sulfamoylpropan-2-yl)amino)propyl)pyrrolidine-1-carboxylate (28.5 mg, 0.059 mmol) was dissolved in methanolic HCl (3 M, 3 ml). The solvent was slowly removed under N₂ stream followed by under reduced pressure overnight to afford (2R,3R)-3-methoxy-2-methyl-N—((S)-1-phenyl-3-sulfamoylpropan-2-yl)-3-((S)-pyrrolidin-2-yl)propanamide as HCl salt. MS m/z 384.2 (M+1). Retention time 0.630 min.

Step 3: To Cbz-Val-Dil-OH (28.7 mg, 0.066 mmol) in DMF (1 ml) were added DIEA (0.048 ml) and HATU (22.9 mg, 0.060 mmol). The reaction was stirred for 15 min, and then (2R,3R)-3-methoxy-2-methyl-N—((S)-1-phenyl-3-sulfamoylpropan-2-yl)-3-((S)-pyrrolidin-2-yl)propanamide (23 mg, 0.055 mmol) in DMF (1 ml) was added. The reaction mixture was stirred at rt for 2 h, and purified by preparative HPLC (20-70% acetonitrile-H₂O containing 0.05% TFA) to obtain benzyl ((S)-1-(((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)carbamate. MS m/z 802.4 (M+1). Retention time 1.298 min.

Step 4: The product obtained in step 3 (24.6 mg, 0.031 mmol), 10% Pd-C (32.7 mg) and EtOAc (3 ml) were combined and stirred under hydrogen for 8 h at rt. The reaction mixture was filtered and concentrated to give (S)-2-amino-N-((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)-N,3-dimethylbutanamide. MS m/z 668.4 (M+1). Retention time 0.888 min.

Step 5: (1R,3S,4S)-2-(t-Butoxycarbonyl)-2-azabicyclo[2.2.1]heptane-3-carboxylic acid (7.0 mg, 0.029 mmol), DMF (1 ml), DIEA (0.021 ml) and HATU (10.1 mg, 0.027 mmol) were combined and stirred at rt for 15 min, and then the product obtained in step 4 (16.2 mg, 0.024 mmol) in DMF (1 ml) was added. The reaction mixture was stirred at rt for 2 h and purified by preparative HPLC (30-60% acetonitrile-H₂O containing 0.05% TFA) to obtain (1R,3S,4S)-t-butyl 3-(((S)-1-(((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate. MS m/z 891.5(M+1). Retention time 1.319 min.

Step 6: The product obtained in step 5 (13.2 mg, 0.015 mmol) was dissolved in methanolic HCl (3 M, 3 ml). The solvent was slowly removed under N₂ stream followed by under reduced pressure overnight to afford (1R,3S,4S)—N—((S)-1-(((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (56) as HCl salt. MS m/z 791.5(M+1). Retention time 0.923 min.

### Example 3-17

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(5-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (57)

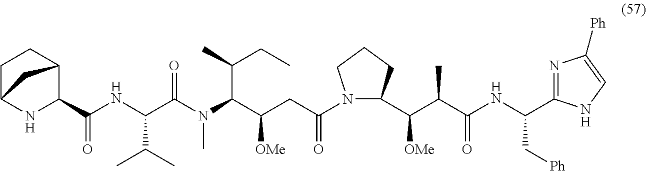 [SMILES: CC[C@H](C)[C@@H]([C@@H](CC(=O)N1CCC[C@H]1[C@H](OC)[C@@H](C)C(=O)N[C@@H](Cc1ccccc1)c1nc(-c2ccccc2)c[nH]1)OC)N(C)C(=O)[C@@H](NC(=O)[C@H]1N[C@@H]2CC[C@H]1C2)C(C)C]

DIEA (33 mg, 0.26 mmol) and HATU (19 mg, 0.051 mmol) was added to (2R,3R)-3-((S)-1-((3R,4S,5S)-4-((S)-2-((1R,3S,4S)-2-(t-butoxycarbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid (40 mg, 0.043 mmol) in DMF (2 ml). The reaction was stirred at rt for 15 min and then (S)-2-phenyl-1-(5-phenyl-1H-imidazol-2-yl)ethanamine (22.4 mg, 0.085 mmol) was added. The reaction was stirred at rt for 1 h. The crude was purified by preparative HPLC (20-70% acetonitrile-H₂O containing 0.05% TFA) to obtain (1R,3S,4S)-t-butyl 3-(((S)-1-(((3R,4S,5S)-3-methoxy-1-((S)-2-((1R,2R)-1-methoxy-2-methyl-3-oxo-3-(((S)-2-phenyl-1-(5-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate. MS m/z 940.5 (M+1). Retention time 1.333 min. This product (13.9 mg, 0.015 mmol) was dissolved in methanolic HCl (3 M, 3 ml). The solvent was slowly removed under stream of N₂ followed by under reduced pressure overnight to afford compound (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(5-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (57) as HCl salt. MS m/z 840.5 (M+1). Retention time 0.936 min.

### Example 3-18

### (1R,3S,4S)—N—((S)-1-(((3R,4S,5S)-3-Methoxy-1-((S)-2-((1R,2R)-1-methoxy-3-(((S)-1-methoxy-3-phenylpropan-2-yl)amino)-2-methyl-3-oxopropyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (58)

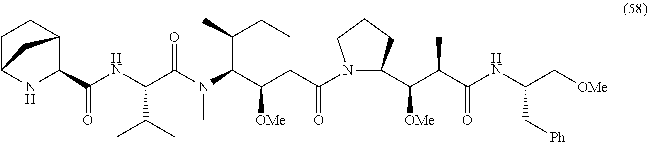 [SMILES: CC[C@H](C)[C@@H]([C@@H](CC(=O)N1CCC[C@H]1[C@H](OC)[C@@H](C)C(=O)N[C@H](COC)Cc1ccccc1)OC)N(C)C(=O)[C@@H](NC(=O)[C@H]1N[C@@H]2CC[C@H]1C2)C(C)C]

Compound (58) was prepared by the procedure described for compound (57) using (S)-1-methoxy-3-phenylpropan-2-amine HCl salt in place of (S)-2-phenyl-1-(5-phenyl-1H-imidazol-2-yl)ethanamine. MS m/z 742.5(M+1). Retention time 0.997 min.

### Example 3-19

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(1H-pyrazol-3-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (59)

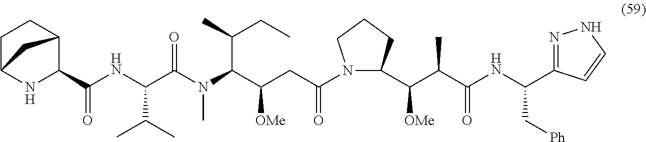 [SMILES: CC[C@H](C)[C@@H]([C@@H](CC(=O)N1CCC[C@H]1[C@H](OC)[C@@H](C)C(=O)N[C@@H](Cc1ccccc1)c1cc[nH]n1)OC)N(C)C(=O)[C@@H](NC(=O)[C@H]1N[C@@H]2CC[C@H]1C2)C(C)C]

Compound (59) was prepared by the procedure described for compound (57) using (S)-2-phenyl-1-(1H-pyrazol-3-yl)ethanamine HCl salt in place of (S)-2-phenyl-1-(5-phenyl-1H-imidazol-2-yl)ethanamine. MS m/z 764.5(M+1). Retention time 0.959 min.

### Example 3-20

### (1R,3S,4S)—N—((S)-1-(((3R,4S,5S)-3-Methoxy-1((S)-2-((1R,2R)-1-methoxy-2-methyl-3-oxo-3-(((S)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (60), and (1R,3S,4S)—N—((S)-1-(((3R,4S,5S)-3-methoxy-1-((S)-2-((1R,2R)-1-methoxy-2-methyl-3-oxo-3-(((R)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (61)

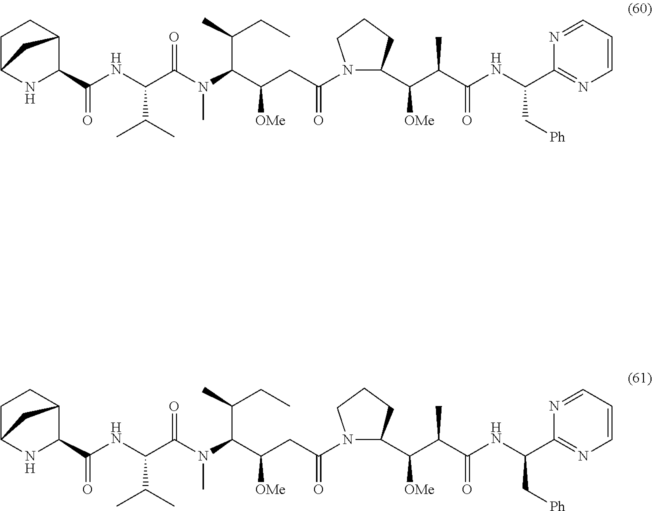 [SMILES: CC[C@H](C)[C@@H]([C@@H](CC(=O)N1CCC[C@H]1[C@H](OC)[C@@H](C)C(=O)N[C@@H](Cc1ccccc1)c1ncccn1)OC)N(C)C(=O)[C@@H](NC(=O)[C@H]1N[C@@H]2CC[C@H]1C2)C(C)C ; CC[C@H](C)[C@@H]([C@@H](CC(=O)N1CCC[C@H]1[C@H](OC)[C@@H](C)C(=O)N[C@H](Cc1ccccc1)c1ncccn1)OC)N(C)C(=O)[C@@H](NC(=O)[C@H]1N[C@@H]2CC[C@H]1C2)C(C)C]

Compounds (60) and (61) were prepared by the procedure described for compound (57) using 2-phenyl-1-(pyrimidin-2-yl)ethanamine in place of (S)-2-phenyl-1-(5-phenyl-1H-imidazol-2-yl)ethanamine. Boc protected (60) and (61) were separated on preparative HPLC (30-65% acetonitrile-H₂O containing 0.05% TFA). Removal of the Boc group from Boc protected (60) and (61) afforded ((1R,3S,4S)—N—((S)-1-(((3R,4S,5S)-3-methoxy-1-((S)-2-((1R,2R)-1-methoxy-2-methyl-3-oxo-3-(((S)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (60) and (1R,3S,4S)—N—((S)-1-(((3R,4S,5S)-3-methoxy-1-((S)-2-((1R,2R)-1-methoxy-2-methyl-3-oxo-3-(((R)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (61) as a HCl salt, respectively. MS m/z 776.5(M+1). Retention time 1.001 min (60) and 1.016min (61).

### Example 3-21

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(5-phenyl-1,3,4-oxadiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (62)

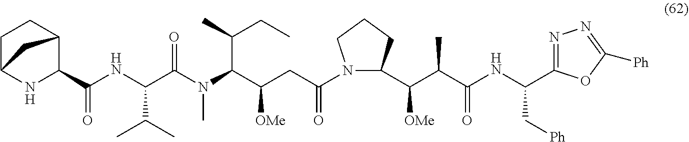 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@H]1N[C@@H]2CC[C@H]1C2)C(C)C]

Compound (62) was prepared in the procedure described for compound (57) using (S)-2-phenyl-1-(5-phenyl-1,3,4-oxadiazol-2-yl)ethanamine in place of (S)-2-phenyl-1-(5-phenyl-1H-imidazol-2-yl)ethanamine with in step 1. After removal of the Boc group, compound (62) was obtained as HCl salt. MS m/z 842.5(M+1). Retention time 1.112 min.

### Example 3-22

### (1R,3S,4S)-2-(Cyanomethyl)-N—((S)-1-(((3R,4S,5S)-1-((S)-2-((1R,2R)-3-(((S)-1-hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (63)

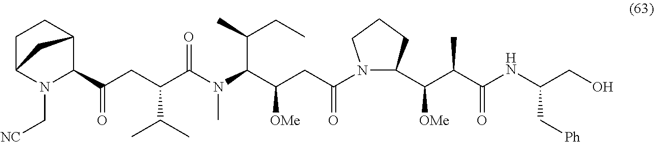 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](CC(=O)[C@@H]1[C@H]2CC[C@H](C2)N1CC#N)C(C)C]

Step 1: DIEA (104 mg, 0.80 mmol) and HATU (122 mg, 0.32 mmol) were added to a solution of (1R,3S,4S)-2-(tert-butoxycarbonyl)-2-azabicyclo[2.2.1]heptane-3-carboxylic acid (78 mg, 0.32 mmol) in DMF (3 ml). The reaction mixture was stirred at rt for 5 min and then added to Val-Dil-Dap-OMe (130 mg, 0.27 mmol) in DMF (2 ml). The reaction mixture was then stirred at rt for 1 h and concentrated. Saturated sodium bicarbonate solution (5 ml) was added to the residue and the product was extracted with DCM (10 ml×3). The organic layers were combined, dried and concentrated to obtain (1R,3S,4S)-t-butyl 3-(((S)-1-(((3R,4S,5S)-1-((S)-2-((1R,2R)-1,3-dimethoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate. The product was used in the next step without further purification. MS m/z 710.5 (M+H). Retention time 1.440 min.

Step 2: The product obtained in step 1 (190 mg, 0.27 mmol) in DCM (10 ml) was treated with TFA (2 ml) at rt for 3 h, and then concentrated to give (2R,3R)-methyl 3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate as TFA salt. MS m/z 610.5 (M+H). Retention time 1.003 min. The product was used in the next step without further purification.

Step 3: To the product obtained in step 2 (193 mg, 0.27 mmol) in MeOH (10 ml) were added acetic acid (0.015 ml, 0.27 mmol), paraformaldehyde (40 mg, 1.3 mmol) and sodium cyanoborohydride (84 mg, 1.4 mmol). The reaction was stirred at 50° C. for 16 h. LCMS indicated that approximately 90% was converted to the cyanomethylated compound and about 10% was converted to the methylated compound. The reaction mixture was filtered and purified by preparative HPLC (20-60% acetonitrile-H₂O containing 0.05% TFA). Fractions containing the cyano adduct were collected and concentrated to give (2R,3R)-methyl 3-((S)-1-((3R,4S,5S)-4-((S)-2-((1R,3S,4S)-2-(cyanomethyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanoate as TFA salt. MS m/z 648.5 (M+H). Retention time 1.261 min.

Step 4: To the product (0.12 g, 0.16 mmol) obtained in step 3 in MeOH—H₂O (1:1 5 ml) was added LiOH (50 mg, 2.09 mmol). The reaction mixture was stirred at rt for 16 h and then acidified with 0.2 ml 10% HCl. The cyano group was partially hydrolyzed to form a carbamoylmethylated product in addition to the cyanomethyl one. The reaction was concentrated and the two products were isolated by preparative HPLC (20-50% acetonitrile-H₂O containing 0.05% TFA) to obtain (2R,3R)-3-((S)-1-((3R,4S,5S)-4-((S)-2-((1R,3S,4S)-2-(cyanomethyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid, MS m/z 634.4 (M+H), retention time 1.138 min, and (2R,3R)-3-((S)-1-((3R,4S,5S)-4-((S)-2-((1R,3S,4S)-2-(2-amino-2-oxoethyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid as TFA salts. MS m/z 652.4 (M+H). Retention time 0.888 min.

Step 5: To (2R,3R)-3-((S)-1-((3R,4S,5S)-4-((S)-2-((1R,3S,4S)-2-(cyanomethyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid TFA salt (6 mg, 0.008 mmol) in DMF was added DIEA (3.1 mg, 0.024 mmol) and HATU (3.7 mg, 0.0096 mmol). The reaction was stirred at rt for 5 min and then (S)-2-amino-3-phenylpropan-1-ol (2.4 mg, 0.016 mmol) was added. The reaction was stirred at rt for 1 h. The crude was purified by preparative HPLC (10-60% acetonitrile-H₂O containing 0.05% TFA) to obtain compound (63) as TFA salt. MS m/z 767.5 (M+H). Retention time 1.189 min.

### Example 3-23

### (1R,3S,4S)-2-(2-Amino-2-oxoethyl)-N—((S)-1-(((3R,4S,5S)-1-((S)-2-((1R,2R)-3-(((S)-1-hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (64)

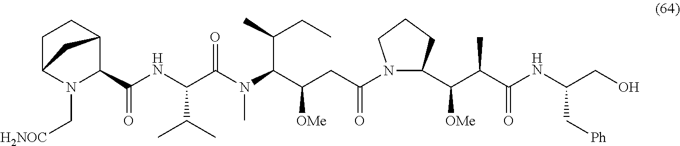 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@H]2CC[C@H](C2)N1CC(N)=O)C(C)C]

To (2R,3R)-3-((S)-1-((3R,4S,5S)-4-((S)-2-((1R,3S,4S)-2-(2-amino-2-oxoethyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid TFA salt (step 4, Example 3-22) (6.1 mg, 0.008 mmol) in DMF were added DIEA (3.1 mg, 0.024 mmol) and HATU (3.7 mg, 0.0096 mmol). The reaction was stirred at rt for 5 min and then (S)-2-amino-3-phenylpropan-1-ol (2.4 mg, 0.016 mmol) was added. The reaction was stirred at rt for 1 h. The crude was purified by preparative HPLC (10-60% acetonitrile-H₂O containing 0.05% TFA) to obtain compound (64) as TFA salt. MS m/z 785.5 (M+H). Retention time 0.951 min.

### Example 3-24

### (1R,3S,4S)-2-Acetyl-N—((S)-1-(((3R,4S,5S)-1-((S)-2-((1R,2R)-3-(((S)-1-hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (65)

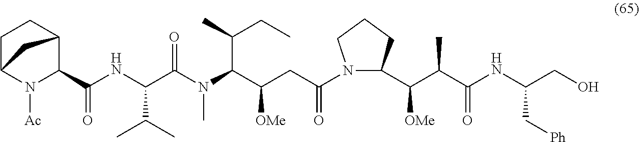 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@H]2CC[C@H](C2)N1C(C)=O)C(C)C]

Step 1: To (2R,3R)-methyl 3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate (step 2, Example 3-63) (13 mg, 0.021 mmol) TFA salt in DCM (2 ml) were added DIEA (0.014 ml, 0.082 mmol) and acetic anhydride (0.0039 ml, 0.041 mmol). The reaction was stirred at rt for 1 h. Aqueous Na₂CO₃ (2 M) was added and the reaction mixture was extracted with DCM (5 ml×3). The organic layers were combined, dried over Na₂SO₄, filtered and then concentrated to give (2R,3R)-methyl 3-((S)-1-((3R,4S,5S)-4-((S)-2-((1R,3S,4S)-2-acetyl-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoate. The product was used in the next step without further purification. MS m/z 651.5 (M+H). Retention time 1.188 min.

Step 2: To the product obtained in step 1 in MeOH:H₂O (1:1 2 ml) was added LiOH (10 mg, 0.42 mmol). The reaction was stirred at rt for 16 h. The reaction mixture was concentrated and 0.040 ml HOAc was added. The crude was purified by preparative HPLC (10-50% acetonitrile-H₂O containing 0.05% TFA) to obtain (2R,3R)-3-((S)-1-((3R,4S,5S)-4-((S)-2-((1R,3S,4S)-2-acetyl-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanoic acid. MS m/z 637.4 (M+H). Retention time 1.158 min.

Step 3: To a solution of the product obtained in step 2, (5 mg, 0.008 mmol) in DMF (1 ml) were added DIEA (2.7 mg, 0.021 mmol) and HATU (3.9 mg, 0.010 mmol). The reaction was stirred at rt for 5 min and then (S)-2-amino-3-phenylpropan-1-ol (1.6 mg, 0.010 mmol) was added. The reaction was stirred at rt for 1 h and then the crude was purified by preparative HPLC (10-60% acetonitrile-H₂O containing 0.05% TFA) to obtain compound (65). MS m/z 770.5 (M+H). Retention time 1.121 min.

### Example 3-25

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(4-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (66)

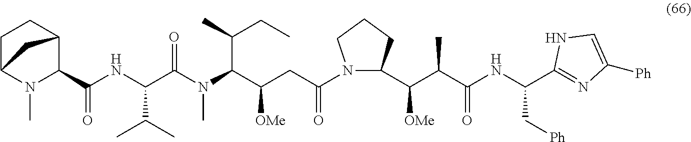 [SMILES: CC[C@H](C)[C@@H]([C@@H](CC(=O)N1CCC[C@H]1[C@H](OC)[C@@H](C)C(=O)N[C@@H](Cc1ccccc1)c1nc(-c2ccccc2)c[nH]1)OC)N(C)C(=O)[C@@H](NC(=O)[C@@H]1[C@H]2CC[C@H](C2)N1C)C(C)C]

DIEA (0.0097 ml) and HATU (3.2 mg, 0.0083 mmol) were added to (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid TFA salt (Step 2, Example 3-12), 4.0 mg, 0.0055 mmol) in DMF (0.5 ml). The reaction was stirred for 15 min at rt, and (S)-2-Phenyl-1-(4-phenyl-1H-imidazol-2-yl)ethanamine (2.9 mg, 0.011 mmol) in DMF (0.5 ml) was added. The reaction mixture was stirred for 2 h at rt and then purified by preparative HPLC (20-70% acetonitrile-H₂O containing 0.05% TFA) to obtain (66). MS m/z 854.5 (M+1). Retention time 0.980 min.

### Example 3-26

### (1R,3S,4S)—N—((S)-1-(((3R,4S,5S)-3-Methoxy-1-((S)-2-((1R,2R)-1-methoxy-3-(((S)-1-methoxy-3-phenylpropan-2-yl)amino)-2-methyl-3-oxopropyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (67)

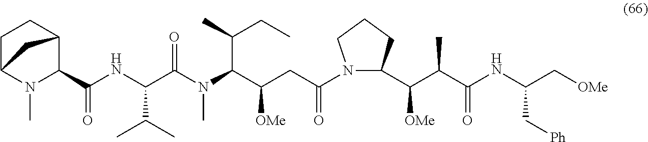 [SMILES: CC[C@H](C)[C@@H]([C@@H](CC(=O)N1CCC[C@H]1[C@H](OC)[C@@H](C)C(=O)N[C@H](COC)Cc1ccccc1)OC)N(C)C(=O)[C@@H](NC(=O)[C@@H]1[C@H]2CC[C@H](C2)N1C)C(C)C]

Compound (67) was obtained by the method described for compound (66) using (S)-1-methoxy-3-phenylpropan-2-amine HCl salt in place of (S)-2-phenyl-1-(5-phenyl-1H-imidazol-2-yl)ethanamine. MS m/z 756.5 (M+1). Retention time 1.046 min.

### Example 3-27

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(1H-pyrazol-3-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (68)

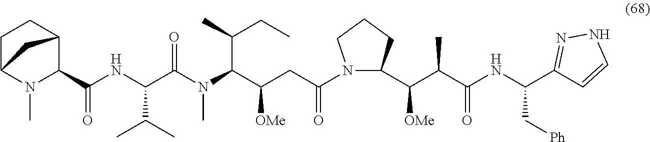 [SMILES: CC[C@H](C)[C@@H]([C@@H](CC(=O)N1CCC[C@H]1[C@H](OC)[C@@H](C)C(=O)N[C@@H](Cc1ccccc1)c1cc[nH]n1)OC)N(C)C(=O)[C@@H](NC(=O)[C@@H]1[C@H]2CC[C@H](C2)N1C)C(C)C]

Compound (68) was obtained by the method described for compound (66) using (S)-2-phenyl-1-(1H-pyrazol-3-yl)ethanamine HCl salt in place of (S)-2-phenyl-1-(5-phenyl-1H-imidazol-2-yl)ethanamine. MS m/z 778.5 (M+1). Retention time 0.998 min.

### Example 3-28

### (1R,3S,4S)—N—((S)-1-(((3R,4S,5S)-3-Methoxy-1-((S)-2-((1R,2R)-1-methoxy-2-methyl-3-oxo-3-(((S)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.1.1]heptane-3-carboxamide (69) and (1R,3S,4S)—N—((S)-1-(((3R,4S,5S)-3-methoxy-1-((S)-2-((1R,2R)-1-methoxy-2-methyl-3-oxo-3-(((R)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (70)

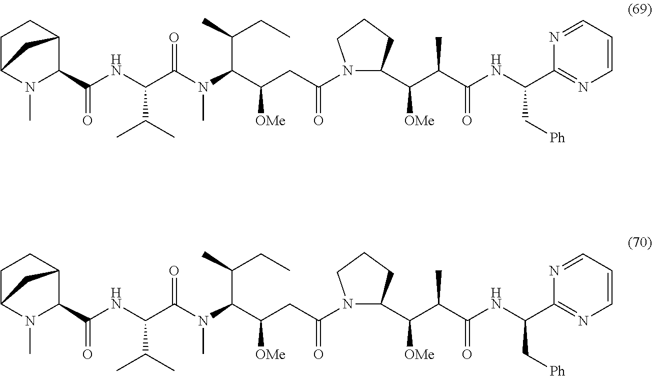 [SMILES: CC[C@H](C)[C@@H]([C@@H](CC(=O)N1CCC[C@H]1[C@H](OC)[C@@H](C)C(=O)N[C@@H](Cc1ccccc1)c1ncccn1)OC)N(C)C(=O)[C@@H](NC(=O)[C@@H]1[C@H]2CC[C@H](C2)N1C)C(C)C ; CC[C@H](C)[C@@H]([C@@H](CC(=O)N1CCC[C@H]1[C@H](OC)[C@@H](C)C(=O)N[C@H](Cc1ccccc1)c1ncccn1)OC)N(C)C(=O)[C@@H](NC(=O)[C@@H]1[C@H]2CC[C@H](C2)N1C)C(C)C]

Compounds (69) and (70) were obtained by the method described for compound (66) using 2-phenyl-1-(pyrimidin-2-yl)ethanamine in place of (S)-2-phenyl-1-(5-phenyl-1H-imidazol-2-yl)ethanamine after preparative HPLC separation (30-55% acetonitrile-H2O containing 0.05% TFA) of the two diasteromers. MS m/z 790.5 (M+1). Retention time 1.016 min and 1.043 min for (69) and (70), respectively.

### Example 3-29

### (1R,3S,4S)—N—((S)-1-(((3R,4S,5S)-3-methoxy-1-((S)-2-((1R,2R)-1-Methoxy-2-methyl-3-oxo-3-(((S)-2-phenyl-1-(5-phenyl-1,3,4-oxadiazol-2-yl)ethyl)amino)propyl)pyrrolidin-1-yl)-5-methyl-1-oxoheptan-4-yl)(methyl)amino)-3-methyl-1-oxobutan-2-yl)-2-methyl-2-azabicyclo[2.1.1]heptane-3-carboxamide (71)

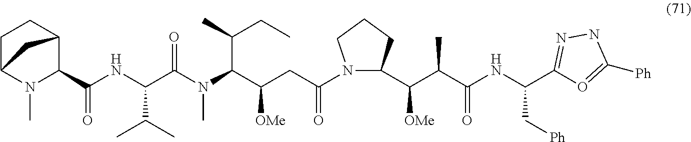

Compound (71) was obtained by the method described for compound (66) using (S)-2-phenyl-1-(5-phenyl-1,3,4-oxadiazol-2-yl)ethanamine in place of (S)-2-phenyl-1-(5-phenyl-1H-imidazol-2-yl)ethanamine. MS m/z 856.5 (M+1). Retention time 1.120 min.

### Example 3-30

### (1R,3S,4S)—N—((S)-1-(((3R,4S,5S)-1-((S)-2-((1R,2R)-3-(((S)-1-(3-Aminophenyl)-3-methoxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (72)

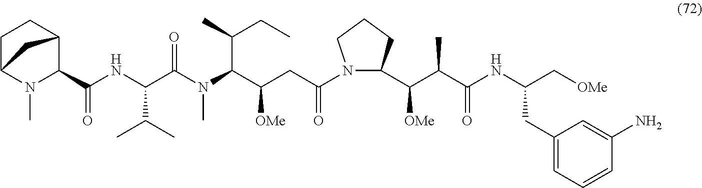 [SMILES: CC[C@H](C)[C@@H]([C@@H](CC(=O)N1CCC[C@H]1[C@H](OC)[C@@H](C)C(=O)N[C@H](COC)Cc1cccc(N)c1)OC)N(C)C(=O)[C@@H](NC(=O)[C@@H]1[C@H]2CC[C@H](C2)N1C)C(C)C]

DIEA (0.012 ml, 0.069 mmol) and HATU (7.89 mg, 0.021 mmol) were added to (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid (Step 2, Example 3-12) (10 mg, 0.014 mmol) in DMF (2 ml). The reaction was stirred at rt for 5 min and then (S)-t-butyl (3-(2-amino-3-methoxypropyl)phenyl)carbamate TFA salt (10.9 mg, 0.028 mmol) was added. The reaction was stirred at rt for 1 h and then the crude was purified by preparative HPLC (20-60% acetonitrile-H₂O containing 0.05% TFA) to obtain t-butyl (3-((S)-2-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-methoxypropyl)phenyl)carbamate as TFA salt. MS m/z 871.5 (M+H). Retention time 1.157 min. To this product (13.6 mg, 0.014 mmol) in acetonitrile (2 ml) was added 10% hydrochloric acid (2 ml). The reaction mixture was stirred at rt for 2 h and then concentrated to give (1R,3S,4S)—N—((S)-1-(((3R,4S,5S)-1-((S)-2-((1R,2R)-3-(((S)-1-(3-aminophenyl)-3-methoxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (72) as HCl salt. MS m/z 771.5 (M+H). Retention time 0.883 min.

### Example 3-31

### (1R,3S,4S)—N—((S)-1-(((3R,4S,5S)-1-((S)-2-((1R,2R)-3-(((S)-1-(4-Aminophenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (73)

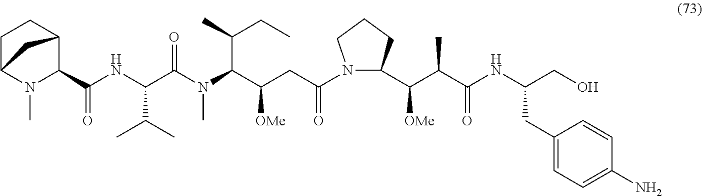 [SMILES: CC[C@H](C)[C@@H]([C@@H](CC(=O)N1CCC[C@H]1[C@H](OC)[C@@H](C)C(=O)N[C@H](CO)Cc1ccc(N)cc1)OC)N(C)C(=O)[C@@H](NC(=O)[C@@H]1[C@H]2CC[C@H](C2)N1C)C(C)C]

Compound (73) was prepared by the method described for compound (72) using (S)-t-butyl (4-(2-amino-3-hydroxypropyl)phenyl)carbamate TFA salt in place of (S)-t-butyl (3-(2-amino-3-methoxypropyl)phenyl)carbamate TFA salt. MS m/z 757.5 (M+H). Retention time 0.787 min.

### Example 3-32

### (1R,3S,4S)—N—((S)-1-(((3R,4S,5S)-1-((S)-2-((1R,2R)-3-(((1S,2R)-1-Hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (74)

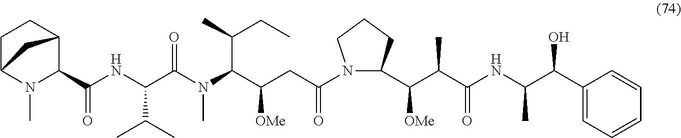 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H]1[C@H]2CC[C@H](C2)N1C)C(C)C]

DIEA (0.006 ml, 0.035 mmol) and HATU (4.0 mg, 0.010 mmol) were added to (2R,3R)-3-((S)-1-((3R,4S,5S)-4-((S)—N,3-dimethyl-2-((1R,3S,4S)-2-methyl-2-azabicycl[2.2.1]heptane-3-carboxamido)butanamido)-3-methoxy-5-methylheptanoyl)pyrrolidin-2-yl)-3-methoxy-2-methylpropanoic acid (5 mg, 0.007 mmol) in DMF (1 ml). The reaction was stirred at rt for 5 min and then (1S,2R)-(+)-norephedrine (3 mg, 0.02 mmol) was added. The reaction mixture was stirred at rt for 1 h and then purified by preparative HPLC (20-50% acetonitrile-H₂O containing 0.05% TFA). Fractions containing the desired product were combined, and 10% hydrochloric acid was added. Concentration afforded compound (74) as HCl salt. MS m/z 742.5 (M+H). Retention time 1.005 min.

### Synthetic Procedure for Example N-Terminal Linked Compounds

### Example 3-33

### (S)-2-((2R,3R)-3-((S)-1-((3R,4S,5S)-4-((S)-2-((1R,3S,4S)-2-(6-(2,5-Dioxo-2,5-dihydro-1H-pyrrol-1-yl)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (10)

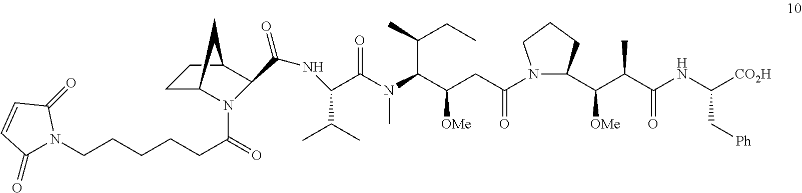 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1C(=O)CCCCCN1C(=O)C=CC1=O)C(C)C]

To a solution of 6-(2,5-dioxo-2,5-dihydro-1H-pyrrol-1-yl)hexanoic acid (EMCA) (1.2 mg, 0.0058 mmol) in DMF (1.0 mL) in a 15 mL round bottom flask was added DIEA (3.0 mg, 0.023 mmol), followed by HATU (2.7 mg, 0.0070 mmol). The reaction mixture was stirred for 10 minutes before a solution of (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (2) (5.0 mg, 0.0058 mmol) in DMF (1.0 mL) was added to The reaction mixture. The reaction mixture was stirred for 1 hour. LCMS analysis indicated the reaction was complete. The crude was purified by reverse phase HPLC, C18 column, eluted with 20-80% acetonitrile-H₂O containing 0.05% TFA. Fractions containing product were concentrated to give compound 10, MS m/z 935.6 (M+1). Retention time 1.17 minutes.

### Example 3-34

### (S)-2-((2R,3R)-3-((S)-1-((3R,4S,5S)-4-((S)-2-((1R,3S,4S)-2-(6-(2,5-Dioxo-2,5-dihydro-1H-pyrrol-1-yl)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (11)

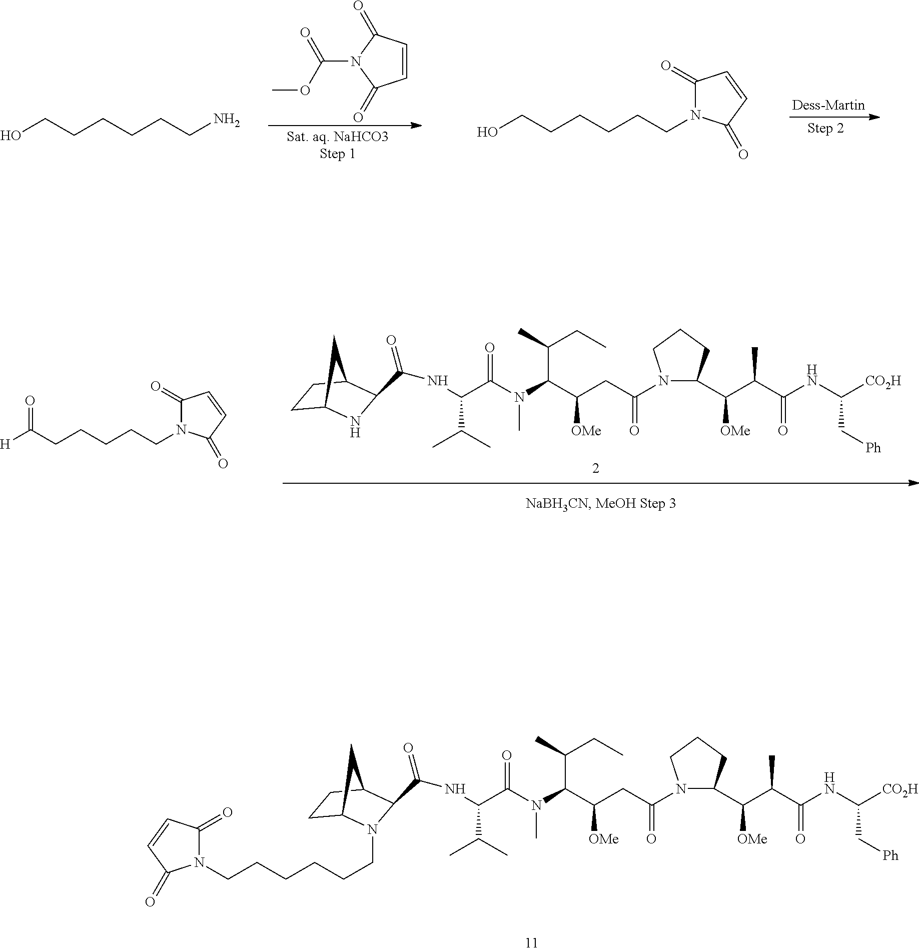 [SMILES: NCCCCCCO ; COC(=O)N1C(=O)C=CC1=O ; O=C1C=CC(=O)N1CCCCCCO ; O=CCCCCCN1C(=O)C=CC1=O ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@H]2CC[C@H]1C2)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1CCCCCCN1C(=O)C=CC1=O)C(C)C]

Step 1: To a 100 mL round bottom flask was added 6-amino-1-hexanol (1.00 g, 6.44 mmol) in saturated NaHCO₃ aqueous solution (12.0 mL). The mixture was cooled at 0° C., and N-methoxycarbonylmaleimide (0.750 g, 6.44 mmol) was added. The reaction mixture was stirred at 0° C. for 1.5 hours. Then the reaction mixture was acidified at 0° C. with 2 M HCl to pH1. The acidified reaction mixture was extracted with ethyl acetate (AcOEt). The organic layer was concentrated. The residue was dissolved in DCM, loaded onto a silica gel column, and eluted with MeOH/DCM (0-4%) to obtain 1-(6-hydroxyhexyl)-1H-pyrrole-2,5-dione as white solid, MS m/z 198.2 (M+1). ¹H NMR (400 MHz, CDCl₃): δ 6.68 (s, 2H), 3.63 (t, d=6.4 Hz, 2H), 3.52 (t, d=7.2 Hz, 2H), 1.63-1.52 (m, 4H), 1.43-1.28 (m, 4H).

Step 2: To 1-(6-hydroxyhexyl)-1H-pyrrole-2,5-dione (237 mg, 1.20 mmol) in DCM (10.0 mL) was added Dess-Martin reagent (618 mg, 1.44 mmol). After 1 hour at room temperature, The reaction mixture was diluted with DCM (10 mL) and filtered. The filtrate was concentrated and purified by ISCO (silicagel, EtOAc/Hexane 0-20%) to afford 6-(2,5-dioxo-2,5-dihydro-1H-pyrrol-1-yl)hexanal as a colorless oil, MS m/z 196.2 (M+1). ¹H NMR (400 MHz, CDCl₃): δ 9.76 (t, J=1.6 Hz, 1H), 6.69 (s, 2H), 3.52 (t, J=7.2 Hz, 2H), 2.43 (td, J=7.2 Hz, 1.6 Hz, 2H), 1.70-1.56 (m, 4H), 1.36-1.28 (m, 2H).

Step 3: Compound 2 (5.0 mg, 0.0067 mmol) and 6-(2,5-dioxo-2,5-dihydro-1H-pyrrol-1-yl)hexanal (6.6 mg, 0.034 mmol) were dissolved in MeOH (1.0 mL). Sodium cyanoborohydride (4.2 mg, 0.067 mmol) was added. The reaction mixture was stirred for 3 hours at room temperature. LCMS analysis indicated the completion of the reaction. The crude was purified by reverse phase HPLC using C18 column, eluted with 10-90% acetonitrile-H₂O containing 0.05% TFA. The factions containing the desired product were pooled and lyophilized to obtain (S)-2-((2R,3R)-3-((S)-1-((3R,4S,5S)-4-((S)-2-((1R,3S,4S)-2-(6-(2,5-dioxo-2,5-dihydro-1H-pyrrol-1-yl)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid 11, MS m/z 921.6(M+1). Retention time 1.07 minutes.

### Example 35

### (S)-2-((2R,3R)-3-((S)-1-((3R,4S,5S)-4-((S)-2-((1R,3S,4S)-2-(((4-((S)-2-((S)-2-(6-(2,5-Dioxo-2,5-dihydro-1H-pyrrol-1-yl)hexanamido)-3-methylbutanamido)-5-ureidopentanamido)benzyl)oxy)carbonyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (12)

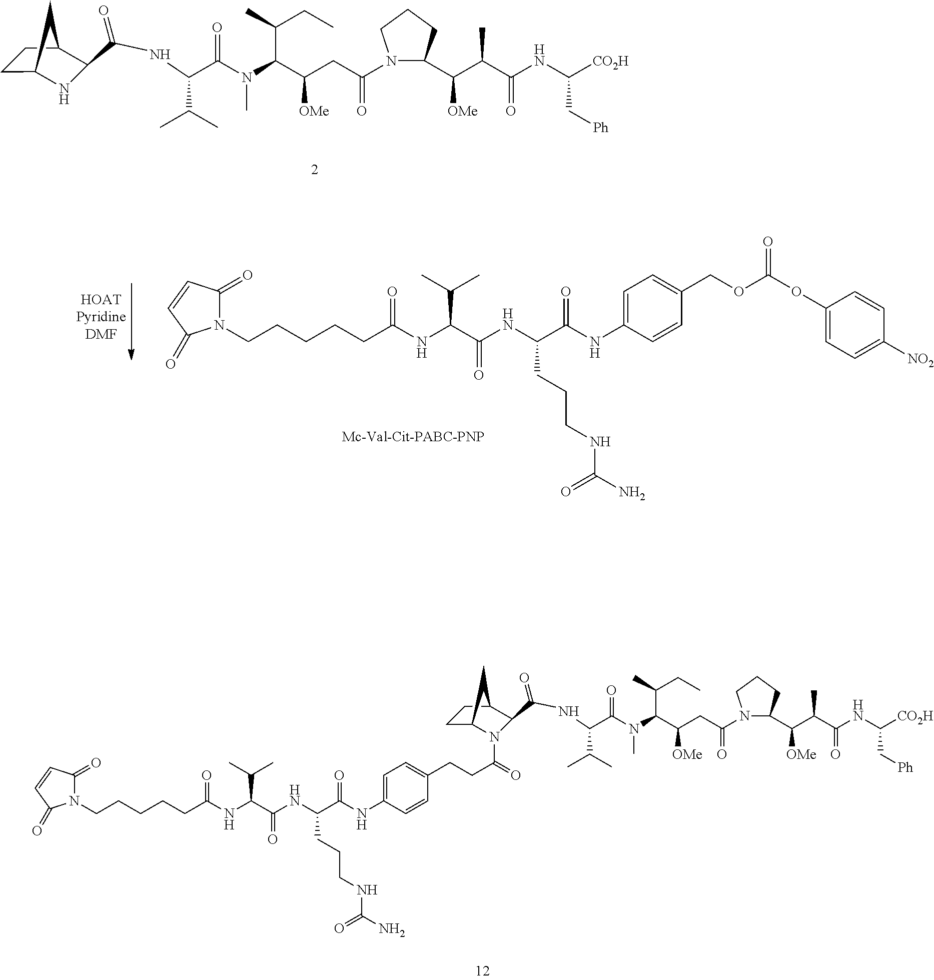 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@H]2CC[C@H]1C2)C(C)C ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1C(=O)CCc1ccc(NC(=O)[C@H](CCCNC(N)=O)NC(=O)[C@@H](NC(=O)CCCCCN2C(=O)C=CC2=O)C(C)C)cc1)C(C)C]

In a 15 mL round bottom flask at room temperature were added MC-Val-Cit-PABC-PNP (5.2 mg, 0.0070 mmol), (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (2) (5.0 mg, 0.0058 mmol) and 1-hydroxy-7-azabenzotriazole (HOAT) (0.6 mg, 0.005 mmol), followed by pyridine-DMF (1:4, 1.25 mL). To the resulting solution was added DIEA (2.3 mg, 0.018 mmol). The reaction mixture was stirred for 72 hours by which time compound 2 was consumed. The reaction mixture mixture was concentrated and the residue was purified by reverse phase HPLC, C18 column, eluted with 20-80% acetonitrile-H₂O, containing 0.05% TFA. The fractions containing the desired product were concentrated to give compound 12, MS m/z 1340.7 (M+1). Retention time 1.15 minutes.

### Example 3-36

### (S)-2-((2R,3R)-3-((S)-1-((3R,4S,5S)-4-((S)-2-((1R,3S,4S)-2-(3-(2-(2-Azidoethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (13)

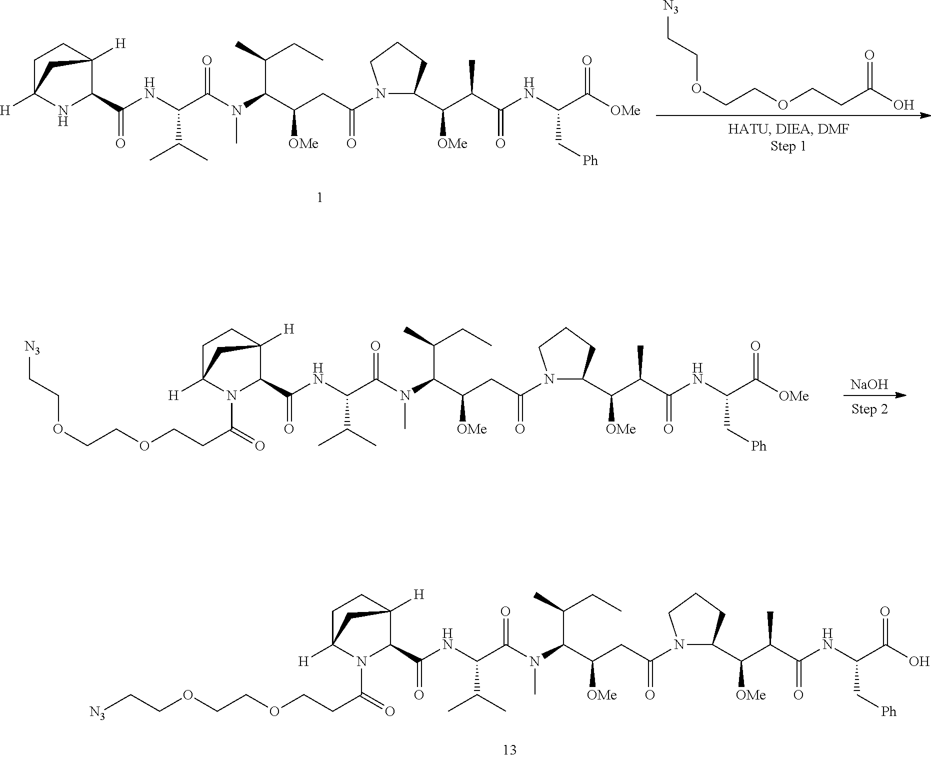

Step 1: To 3-(2-(2-Azidoethoxy)ethoxy)propanoic acid (6.6 mg, 0.033 mmol) in DMF (2 mL) were added DIEA (0.011 mL, 0.065 mmol) and HATU (10.3 mg, 0.027 mmol). After 15 minutes, compound 1 (8.2 mg, 0.010 mmol) was added. The reaction mixture was stirred for 2 hours at room temperature. LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain (S)-methyl 2-((2R,3R)-3-((S)-1-((3R,4S,5S)-4-((S)-2-((1R,3S,4S)-2-(3-(2-(2-azidoethoxy)ethoxy)propanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate, (MS m/z 941.3 (M+1). Retention time 1.30 minutes.

Step 2: The ester product from step 1 was dissolved in acetonitrile (0.3 mL) and H₂O (0.2 mL). Aqueous NaOH (1.0N, 0.15 mL) was added. The reaction mixture was stirred for 30 minutes. The crude was purified by reverse phase HPLC using C18 column, eluted with 10-90% acetonitrile-H₂O containing 0.05% TFA. The factions containing the desired product were pooled and lyophilized to obtain compound 13 (S)-2-((2R,3R)-3-((S)-1-((3R,4S,5S)-4-((S)-2-((1R,3S,4S)-2-(3-(2-(2-azidoethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid, MS m/z 927.5 (M+1). Retention time 1.21 minutes.

### Example 3-37

### (S)-2-((2R,3R)-3-((S)-1-((3R,4S,5S)-4-((S)-2-((1R,3S,4S)-2-(3-(2-(2-(4-((2,5-Dioxo-2,5-dihydro-1H-pyrrol-1-yl)methyl)-1H-1,2,3-triazol-1-yl)ethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (14)

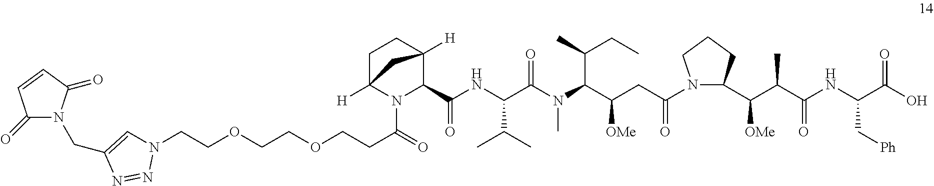 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCOCCOCCn1cc(CN2C(=O)C=CC2=O)nn1)C(C)C]

A solution of (S)-2-((2R,3R)-3-((S)-1-((3R,4S,5S)-4-((S)-2-((1R,3S,4S)-2-(3-(2-(2-azidoethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (13) (5.4 mg, 0.058 mmol),1-(prop-2-yn-1-yl)-1H-pyrrole-2,5-dione (1.6 mg, 0.012 mmol) and CuSO₄ (0.7 mg, 0.005 mmol) in DMF (1.2 mL) and H₂O (0.3 mL) was treated with L-ascorbic acid sodium salt (2.6 mg, 0.015 mmol) and stirred at room temperature for 2 hours. LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain (S)-2-((2R,3R)-3-((S)-1-((3R,4S,5S)-4-((S)-2-((1R,3S,4S)-2-(3-(2-(2-(4-((2,5-dioxo-2,5-dihydro-1H-pyrrol-1-yl)methyl)-1H-1,2,3-triazol-1-yl)ethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid 14, MS m/z 1062.5 (M+1). Retention time 1.15 minutes.

### Example 3-38

### (S)-2-((2R,3R)-3-((S)-1-((3R,4S,5S)-4-((S)—N,3-Dimethyl-2-((1R,3S,4S)-2-(((2-(2-(2-(vinylsulfonyl)ethoxy)ethoxy)ethyl)sulfonyl)ethyl)-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoic acid (15)

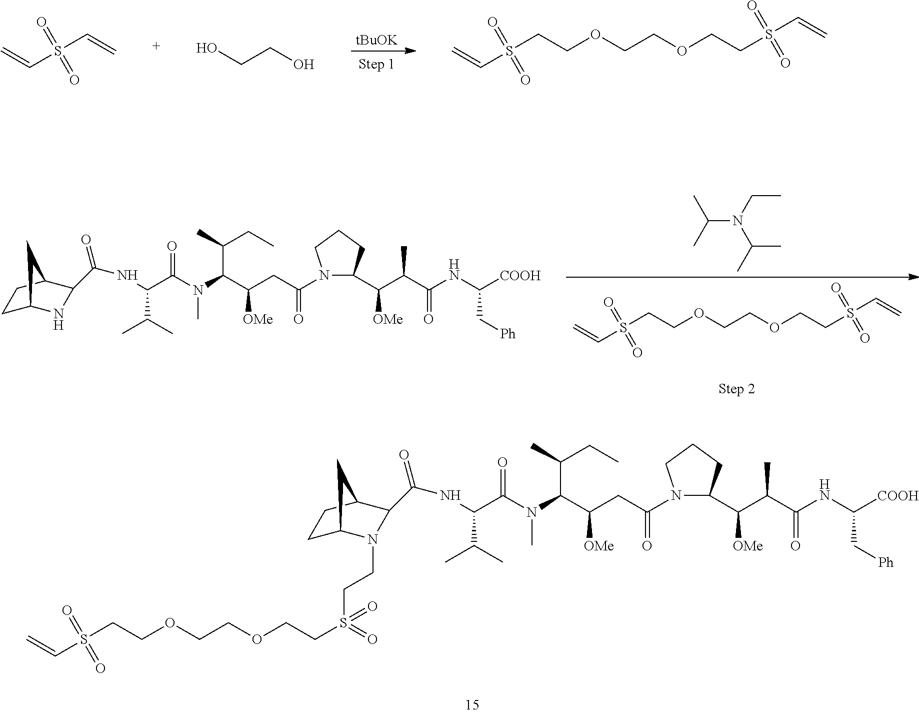

Step 1: t-BuOK (119 mg, 1.10 mmol) was added to a solution of divinyl sulfone (1.60 g, 13.5 mmol) and ethylene glycol (330 mg, 5.32 mmol) in THF (100 mL). The reaction mixture was stirred at room temperature for 18 hours. The solvent was removed under reduced pressure to yield a crude that was purified by silica gel column chromatography (EtOAc-Hexanes 2:1 to 3:1) to give ((2-(2-(2-vinylsulfonylethoxy)ethoxy)ethyl)sulfonyl)ethene as a colorless syrup. ¹H NMR (400 MHz, CDCl₃) d 6.75 (dd, J=9.9 Hz, 16.6 Hz, 2H), 6.39 (d, J=16.6 Hz, 2H), 6.09 (d, J=9.9 Hz, 2H), 3.88 (t, J=5.7 Hz, 4H), 3.61 (s, 4H), 3.24 (t, J=5.7 Hz, 4H).

Step 2: To a solution of ((2-(2-(2-vinylsulfonylethoxy)ethoxy)ethyl)sulfonyl)ethene (13.3 mg, 0.045 mmol) in DCM-i-PrOH (2:1) were added (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (2) (10.0 mg, 0.012 mmol) and DIEA (0.0020 mL, 0.012 mmol). The reaction mixture was heated to 80° C. for 18 hours at which time LCMS analysis indicated the reaction was 70-80% complete. The reaction mixture was concentrated, and the residue was purified by reverse phase HPLC, C18 column, eluted with 10-50% acetonitrile-H₂O, containing 0.05% TFA. The fractions containing he desired product were pooled and concentrated to obtain compound 15 as a TFA salt, MS m/z 1040.4 (M+1). Retention time 1.03 minutes.

### Example 3-39

### (S)-2-((2R,3R)-3-((S)-1-((3R,4S,5S)-4-((S)—N,3-Dimethyl-2-((1R,3S,4S)-2-(3-(Methyl(2-(vinylsulfonyl)ethyl)amino)propanoyl)-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoic acid (16)

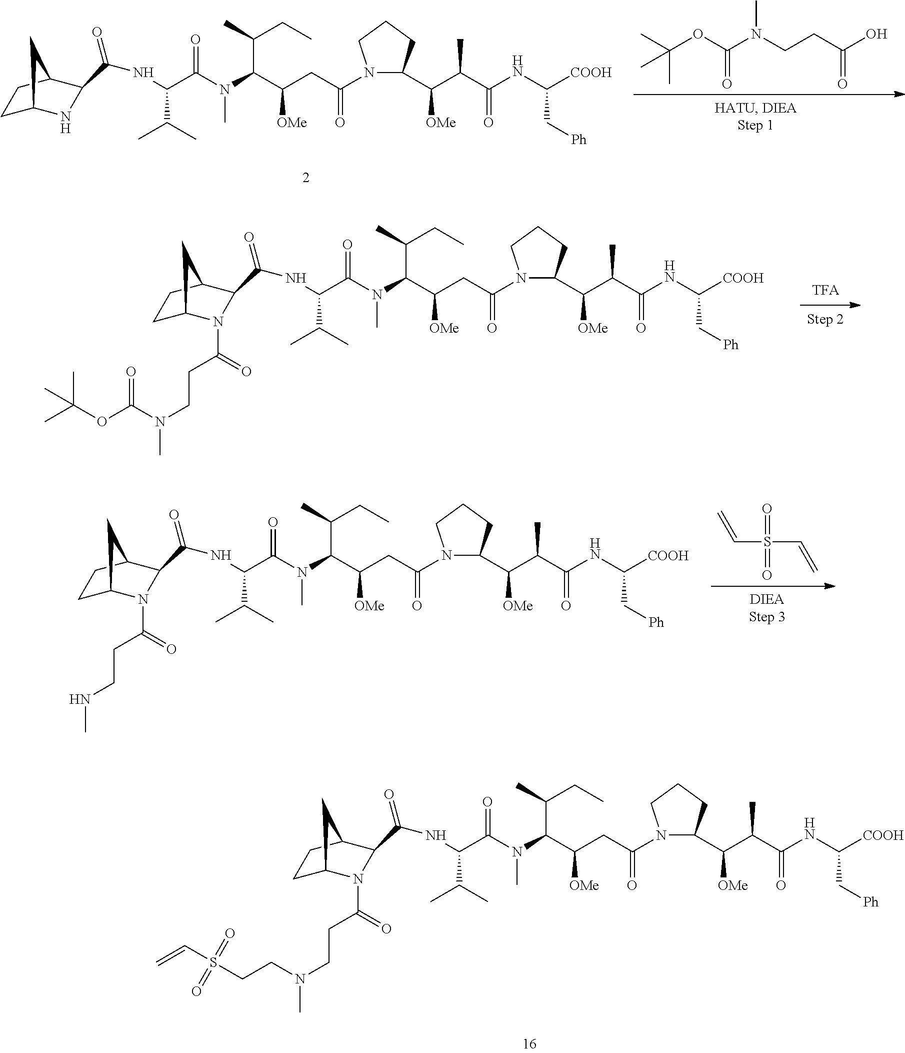

Step 1: To a solution of 3-((tert-butoxycarbonyl)(methyl)amino)propanoic acid (5.4 mg, 0.027 mmol) in DMF (1.0 mL) were added DIEA (0.0070 mL, 0.040 mmol) and HATU (9.1 mg, 0.024 mmol). The reaction mixture was stirred for 5 minutes and (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (2) (11.4 mg, 0.013 mmol) was added. The reaction was complete within 1 hour as judged by LCMS analysis. The crude was purified by reverse phase HPLC, C18 column, eluted with 10-70% acetonitrile-H₂O, containing 0.05% TFA. The fractions containing the desired product were pooled and concentrated to obtain (S)-2-((2R,3R)-3-((S)-1-((3R,4S,5S)-4-((S)-2-((1R,3S,4S)-2-(3-((tert-butoxycarbonyl)(methyl)amino)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid, MS m/z 927.5 (M+1). Retention time 1.28 minutes.

Step 2: To a solution of the product obtained in step 1 (6.4 mg, 0.0069 mmol) in DCM (2.0 mL) was added TFA (1.0 mL). The reaction mixture was stirred at room temperature for 1 hour and concentrated to obtain (S)-2-((2R,3R)-3-((S)-1-((3R,4S,5S)-4-((S)—N,3-dimethyl-2-((1R,3S,4S)-2-(3-(methylamino)propanoyl)-2-azabicyclo[2.1.1]heptane-3-carboxamido)butanamido)-3-methoxy-5-methylheptanoyl)pyrrolidin-2-yl)-3-methoxy-2-methylpropanamido)-3-phenylpropanoic acid TFA salt. MS m/z 827.4 (M+1). Retention time 0.99 minutes. This product was used in the next step without further purification.

Step 3: To a solution of the product TFA salt obtained in step 2 (6.5 mg, 0.0069 mmol) in i-PrOH (2.0 mL) were added divinyl sulfone (20.0 mg, 0.169 mmol) and DIEA (0.010 mL, 0.057 mmol). The reaction mixture was stirred at 80° C. for 1 hour, at which time the reaction was complete as judged by LCMS analysis and the reaction mixture was concentrated. The residue was purified by reverse phase HPLC, C18 column, eluted with 10-60% acetonitrile-H₂O, containing 0.05% TFA. The fractions containing the desired product were pooled and concentrated to obtain compound 16, MS m/z 945.4 (M+1). Retention time 0.99 minutes.

### Example 3-40

### (1R,3S,4S)-2-(6-(2,5-Dioxo-2,5-dihydro-1H-pyrrol-1-yl)hexanoyl)-N—((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (17)

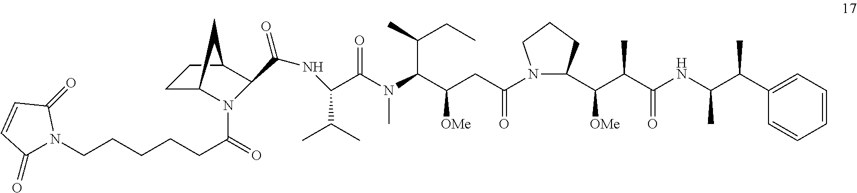 [SMILES: CC[C@H](C)[C@@H]([C@@H](CC(=O)N1CCC[C@H]1[C@H](OC)[C@@H](C)C(=O)N[C@H](C)[C@@H](C)c1ccccc1)OC)N(C)C(=O)[C@@H](NC(=O)[C@@H]1[C@H]2CC[C@@H](C2)N1C(=O)CCCCCN1C(=O)C=CC1=O)C(C)C]

This compound was synthesized using the same method as described for compound (4) (in Example 3-4) from EMCA (5.5 mg, 0.026 mmol), DIEA (10.0 mg, 0.078 mmol), HBTU (9.8 mg, 0.026 mmol) and (1R,3S,4S)—N—((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide TFA salt (3) (21.8 mg, 0.026 mmol). compound 17 was obtained after purification by reverse phase HPLC, MS m/z 921.5 (M+1). Retention time 1.25 minutes.

### Example 3-41

### (S)-2-((2R,3R)-3-((S)-1-((3R,4S,5S)-4-((S)-2-((1R,3S,4S)-2-(6-Mercaptohexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (18)

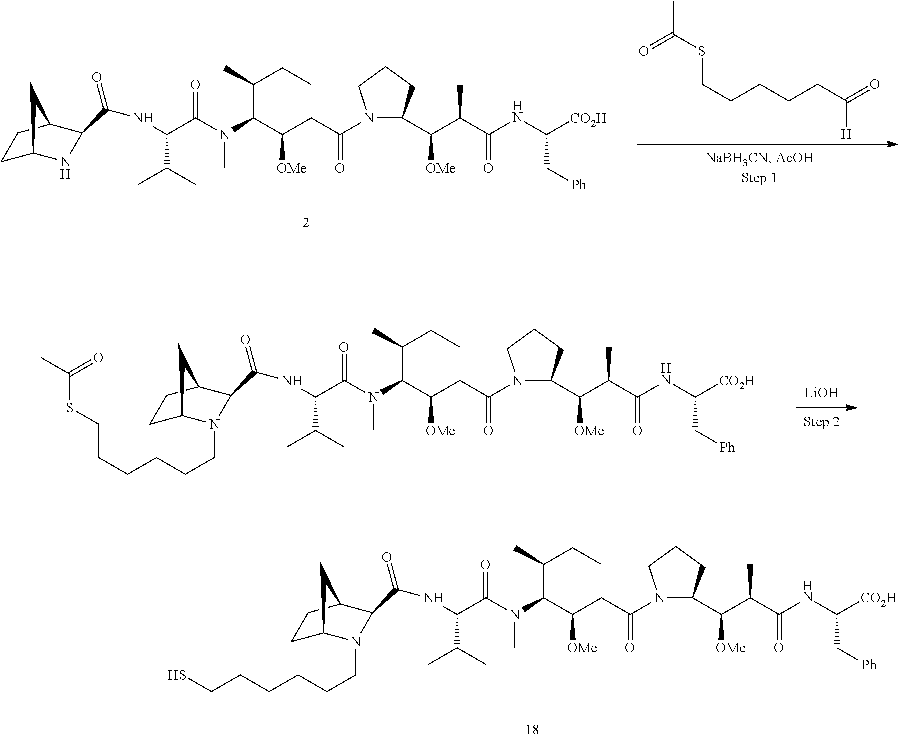 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H]1N[C@H]2CC[C@H]1C2)C(C)C ; CC(=O)SCCCCCC=O ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1CCCCCCSC(C)=O)C(C)C ; "LiOH" ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1CCCCCCS)C(C)C]

Step 1: To a solution of S-(6-oxohexyl) ethanethioate (4.28 mg, 0.025 mmol) and (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (2) (7.0 mg, 0.0082 mmol) in MeOH (2.0 ml) was added acetic acid (0.0050 mL, 0.083 mmol) and sodium cyanoborohydride (2.57 mg, 0.041 mmol). The reaction mixture was heated at 50° C. for 2 hours and the crude was purified by reverse phase HPLC, C18 column, eluted with 20-70% acetonitrile-H₂O, containing 0.05% TFA. The fractions containing the desired product were combined and concentrated, affording (S)-2-((2R,3R)-3-((S)-1-((3R,4S,5S)-4-((S)-2-((1R,3S,4S)-2-(6-(acetylthio)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid, MS m/z 900.5 (M+1), retention time1.17 minutes.

Step 2: The product obtained in step 1 was dissolved in MeOH—H₂O (2:1, 3.0 mL). To the solution was added lithium hydroxide (5.0 mg, 0.21 mmol). The reaction mixture was stirred at room temperature for 0.5 hour and then concentrated to approximatelyl.5 mL. The crude was purified by reverse phase HPLC, C18 column, eluted with 20-60% acetonitrile-H₂O, containing 0.05% TFA. The fractions containing desired product were pooled and concentrated to obtain compound 18, MS m/z 858.5 (M+1). Retention time 1.16 minutes.

### Example 3-42

### (S)-2-((2R,3R)-3-((S)-1-((3R,4S,5S)-4-((S)-2-((1R,3S,4S)-2-(6-(3-Amino-4-formylphenoxy)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (19)

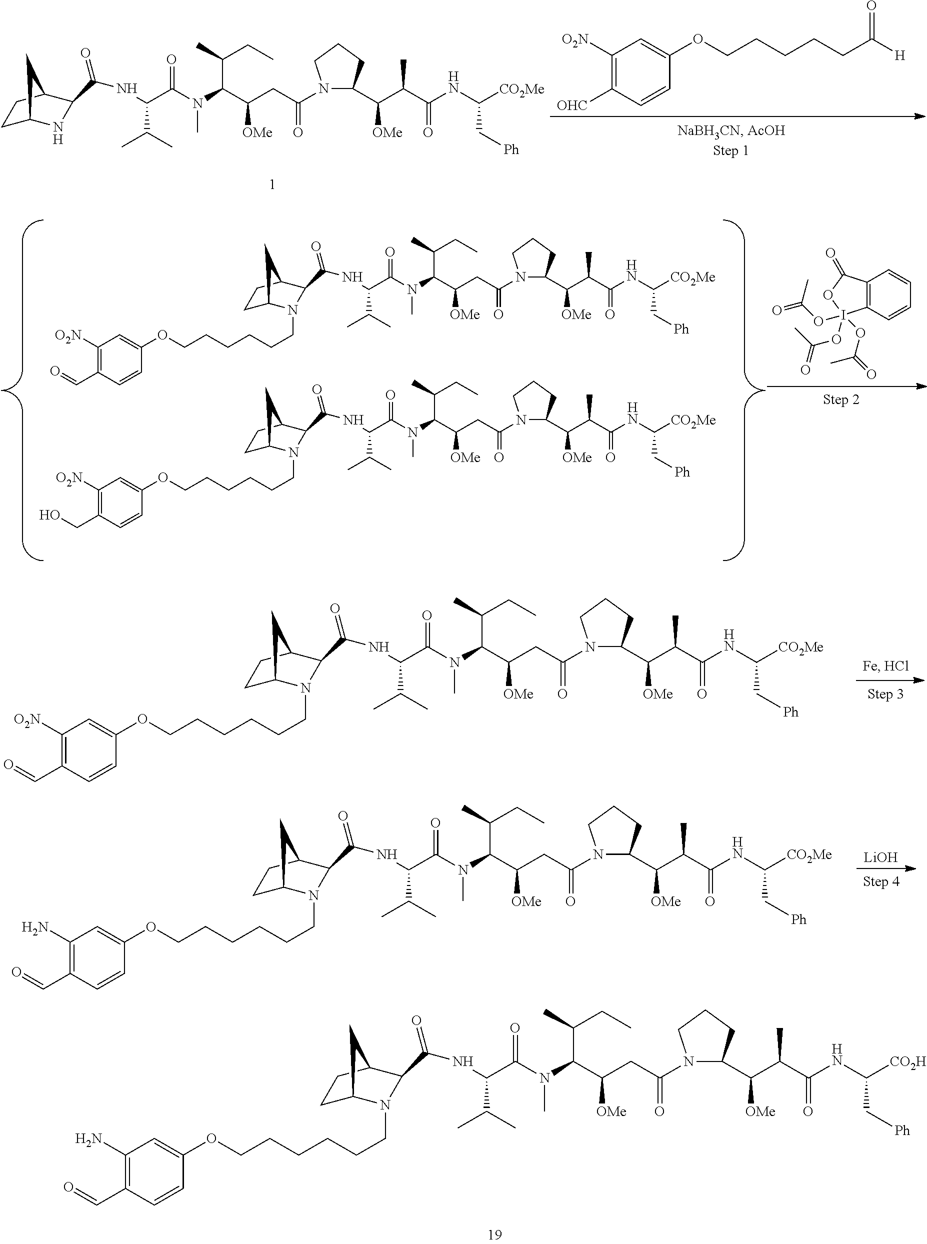

Step 1: To a solution of 2-nitro-4-((6-oxohexyl)oxy)benzaldehyde (20.1 mg, 0.076 mmol) and (S)-methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate (1) (16.5 mg, 0.019 mmol) in DMF (2.0 mL) were added acetic acid (0.0076 mL, 0.13 mmol) and sodium cyanoborohydride (11.9 mg, 0.190 mmol). The reaction mixture was heated at 50° C. for 2 hours and the crude was purified by reverse phase HPLC, C18 column, eluted with 20-70% acetonitrile-H₂O, containing 0.05% TFA. The fractions containing the desired aldehyde (MS m/z 1005.5 (M+1), retention time 1.27 minutes) and the desired alcohol (MS m/z 1007.5 (M+1), retention time 1.21 minutes) intermediates were combined and concentrated and used in the next step.

Step 2: The mixture obtained from step 1 containing the aldehyde and the alcohol was dissolved in DCM (2.0 mL) and Dess-Martin periodinane (4.0 mg, 0.0095 mmol) was added. The reaction mixture was stirred at room temperature for 2 hours. The reaction mixture mixture was then washed with Na₂S₂O₃ aqueous solution and extracted with DCM. The DCM layer was dried over anhydrous Mg Saₜ, filtered and concentrated. The crude was purified by reverse phase HPLC, C18 column, eluted with 20-70% acetonitrile-H₂O, containing 0.05% TFA. The fractions containing the desired product were pooled and concentrated to give (S)-methyl 2-((2R,3R)-3-((S)-1-((3R,4S,5S)-4-((S)-2-((1R,3S,4S)-2-(6-(4-formyl-3-nitrophenoxy)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate TFA salt, MS m/z 1005.5 (M+1). Retention time 1.27 minutes. The product also contained some hydrolyzed acid, MS m/z 991.5 (M+1). Retention time 1.22 minutes.

Step 3: To a solution of the product obtained in step 2 (16.9 mg, 0.015 mmol) in 70% EtOH in water were added iron powder (0.8 mg, 0.02 mmol) and HCl (0.1N, 0.15 mL, 0.015 mmol). The reaction mixture was stirred vigorously at room temperature for 18 hours. Brown precipitate formed. The mixture was filtered through a Celite plug and the filtrate was concentrated. The crude was purified by ISCO, C18 column, eluted with 30-100% acetonitrile-H₂O. The fractions containing the desired product were pooled and concentrated to obtain (S)-methyl 2-((2R,3R)-3-((S)-1-((3R,4S,5S)-4-((S)-2-((1R,3S,4S)-2-(6-(3-amino-4-formylphenoxy)hexyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate, MS m/z 975.5 (M+1). Retention time 1.23 minutes.

Step 4: To a solution of the product obtained in step 3 (4.6 mg, 0.0047 mmol) in MeOH—H₂O (1.5:1, 2.5 mL) was added lithium hydroxide (10.0 mg, 0.435 mmol). The reaction mixture was stirred at room temperature for 4 hours. The reaction mixture was concentrated to about 50% volume and acidified with 1N HCl to pH 5. The crude was purified by ISCO, C18 column, eluted with 30-75% acetonitrile-H₂O. The fractions containing the desired product were pooled, neutralized with 0.3 mg of LiOH, and lyophilized to obtain compound 19, MS m/z 961.5 (M+1). Retention time 1.15 minutes.

### Example 3-43

### Coenzyme A adduct of (S)-2-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.2.1]heptane-3-carboxamido)-N,3-dim ethylbutanamido)-3-methoxy-5-methylheptanoyl)pyrrolidin-2-yl)-3-methoxy-2-methylpropanamido)-3-phenylpropanoic acid (20)

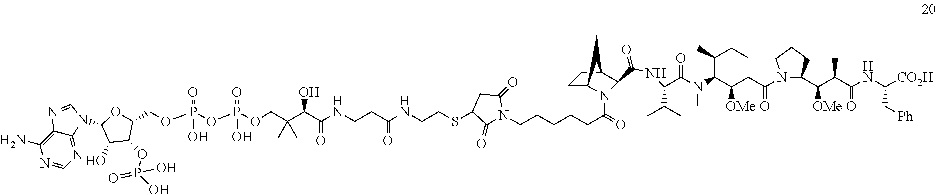 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@@H](C2)N1C(=O)CCCCCN1C(=O)CC(SCCNC(=O)CCNC(=O)[C@H](O)C(C)(C)COP(=O)(O)OP(=O)(O)OC[C@H]2O[C@@H](n3cnc4c(N)ncnc43)[C@@H](O)[C@H]2OP(=O)(O)O)C1=O)C(C)C]

To a solution of Coenzyme A (CoA) trilithium salt (7.6 mg, 0.0096 mmol) in 100 mM phosphate buffer containing 5 mM EDTA at pH7.5 was added a solution of (S)-2-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (17) (9.0 mg, 0.0096 mmol) in DMSO (0.048 mL). The reaction mixture was let stand at room temperature for 1 hour, at which time the reaction was complete as judged by LCMS analysis. The sample was purified by reverse phase HPLC, C18 column, eluted with 20-60% acetonitrile-H₂O, containing 0.05% TFA. The fractions containing the desired product were pooled and concentrated to obtain the CoA adduct 20, MS m/z 852 (M12+1)). Retention time 0.98 minutes.

### Example 3-44

### (S)-2-((2R,3R)-3-((S)-1-((3R,4S,5S)-4-((S)-2-((1R,3S,4S)-2-(6-(2-Bromoacetamido)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (21)

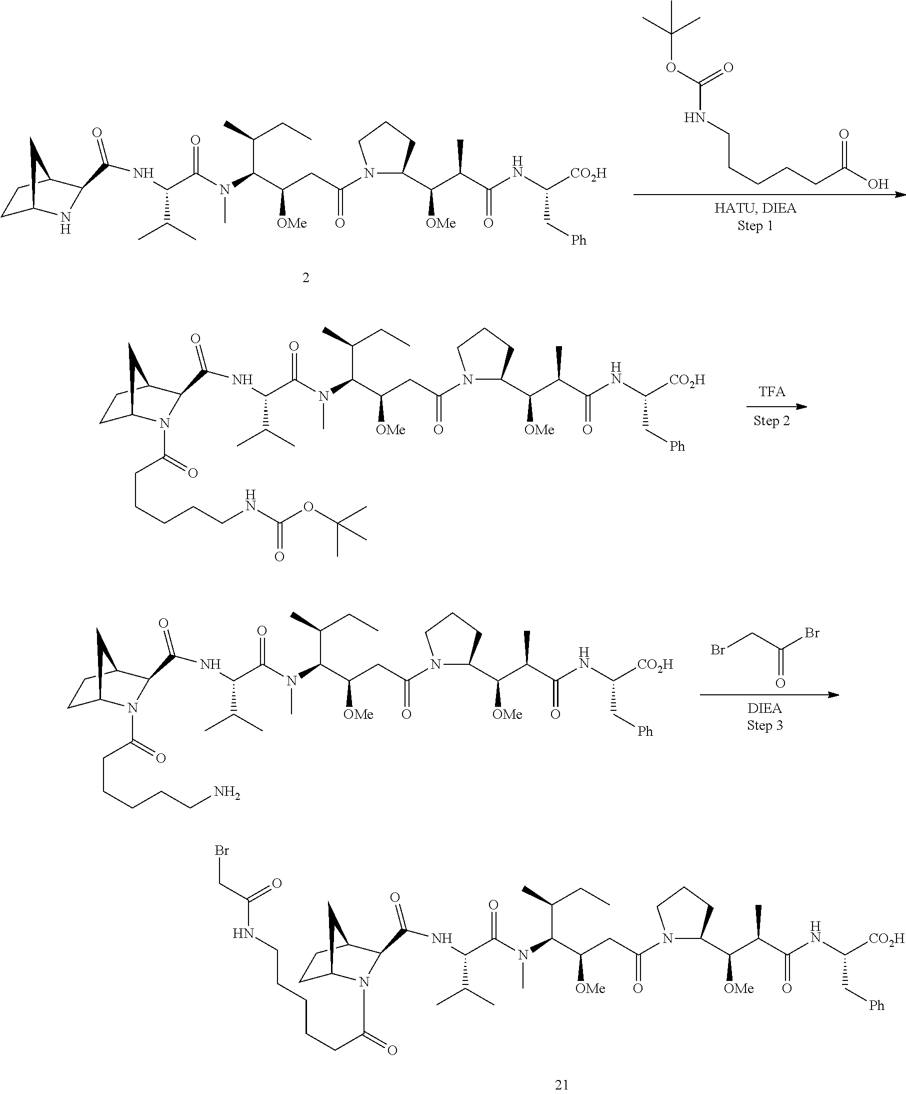

Step 1: To a solution of 6-((tert-butoxycarbonyl)amino)hexanoic acid (12 mg, 0.051 mmol) in DMF (2.0 mL) were added DIEA (18 mg, 0.14 mmol) and HATU (18 mg, 0.047 mmol). The reaction mixture was stirred at room temperature for 10 minutes before (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (2) (40 mg, 0.047 mmol) was added. The reaction was complete within half an hour. The crude was purified by reverse phase HPLC, C18 column, eluted with 20-70% acetonitrile-H₂O, containing 0.05% TFA. The factions containing the desired product were pooled and concentrated to obtain (S)-2-((2R,3R)-3-((S)-1-((3R,4S,5S)-4-((S)-2-((1R,3S,4S)-2-(6-((tert-butoxycarbonyl)amino)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid, MS m/z 955.5 (M+1). Retention time 1.32 minutes.

Step 2: To a solution of the compound obtained in step 1 (15.6 mg, 0.016 mmol) in DCM (2.0 mL) was added TFA (1.0 mL). The reaction mixture was stirred at room temperature for 30 minutes and concentrated to obtain (S)-2-((2R,3R)-3-((S)-1-((3R,4S,5S)-4-((S)-2-((1R,3S,4S)-2-(6-aminohexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt, MS m/z 855.5 (M+1). Retention time 1.01 minutes.

Step 3: (S)-2-((2R,3R)-3-((S)-1-((3R,4S,5S)-4-((S)-2-((1R,3S,4S)-2-(6-aminohexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid TFA salt (20 mg, 0.021 mmol) was dissolved in DCM and treated with DIEA (12 mg, 0.093 mmol). The reaction mixture was cooled to 0° C. To the reaction mixture was then added a solution of 2-bromoacetyl bromide (9.0 mg, 0.045 mmol) in DCM (0.2 mL) with stirring. The reaction mixture was stirred at 0° C. for 10 min and LCMS analysis showed that the amine starting material was consumed. Saturated aqueous NaHCO₃ was added to quench the reaction. The reaction mixture mixture was extracted with DCM (5 mL×3). The organic layers were combined and concentrated. The crude was purified by reverse phase HPLC, C18 column, eluted with 30-45% acetonitrile-H₂O, containing 0.05% TFA. The fractions were pooled and concentrated to obtain compound 21, MS m/z 975.3 (M+1). Retention time 1.19 minutes.

### Example 3-45

### (S)-2-((2R,3R)-3-((S)-1-((3R,4S,5S)-4-((S)-2-((1R,3S,4S)-2-(6-(Aminooxy)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (22)

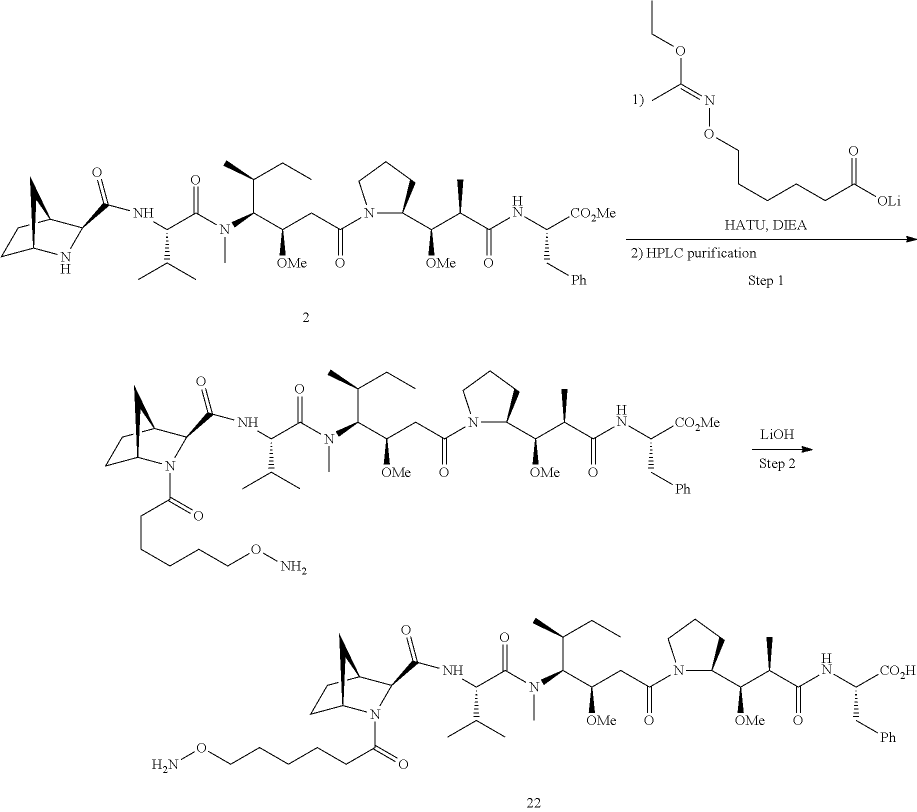

Step 1: To a solution of lithium 6(((1-ethoxyethylidene)amino)oxy)hexanoate (6.3 mg, 0.028 mmol) in DMF (1.0 mL) was added HATU (8.9 mg, 0.023 mmol). The reaction mixture was stirred at room temperature for 20 minutes before the whole reaction mixture was added to a solution of (S)-methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate TFA salt (1) (20 mg, 0.021 mmol) and DIEA (6.0 mg, 0.047 mmol) in DMF (1.0 mL). After stirring at room temperature for 2 hours, the reaction mixture was purified by reverse phase HPLC, C18 column, eluted with 40-80% acetonitrile-H₂O, containing 0.05% TFA. The fractions containing the desired product were pooled and concentrated. LCMS anaylysis revealed that the protecting group on the alkoxylamine moiety was removed to give TFA salt of (S)-methyl 2-((2R,3R)-3-((S)-1-((3R,4S,5S)-4-((S)-2-((1R,3S,4S)-2-(6-(aminooxy)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate, MS m/z 885.5 (M+1). Retention time 1.10 minutes.

Step 2: To a solution of the compound obtained in Step 1 (24.3 mg, 0.023 mmol) in MeOH—H₂O (1:1, 2.0 mL) was added lithium hydroxide (20 mg, 0.84 mmol). The reaction was monitored by LCMS. Upon completion the crude was purified by reverse phase HPLC, C18 column, eluted with 20-40% acetonitrile-H₂O, containing 0.05% TFA. The fractions containing the desired product were concentrated to give compound 22 TFA salt, MS m/z 871.5 (M+1). Retention time 1.03 minutes.

### Example 3-46

### (S)-2-((2R,3R)-3-((S)-1-((3R,4S,5S)-4-((S)-2-((1R,3S,4S)-2-(6-((S)-Aziridine-2-carboxamido)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (23)

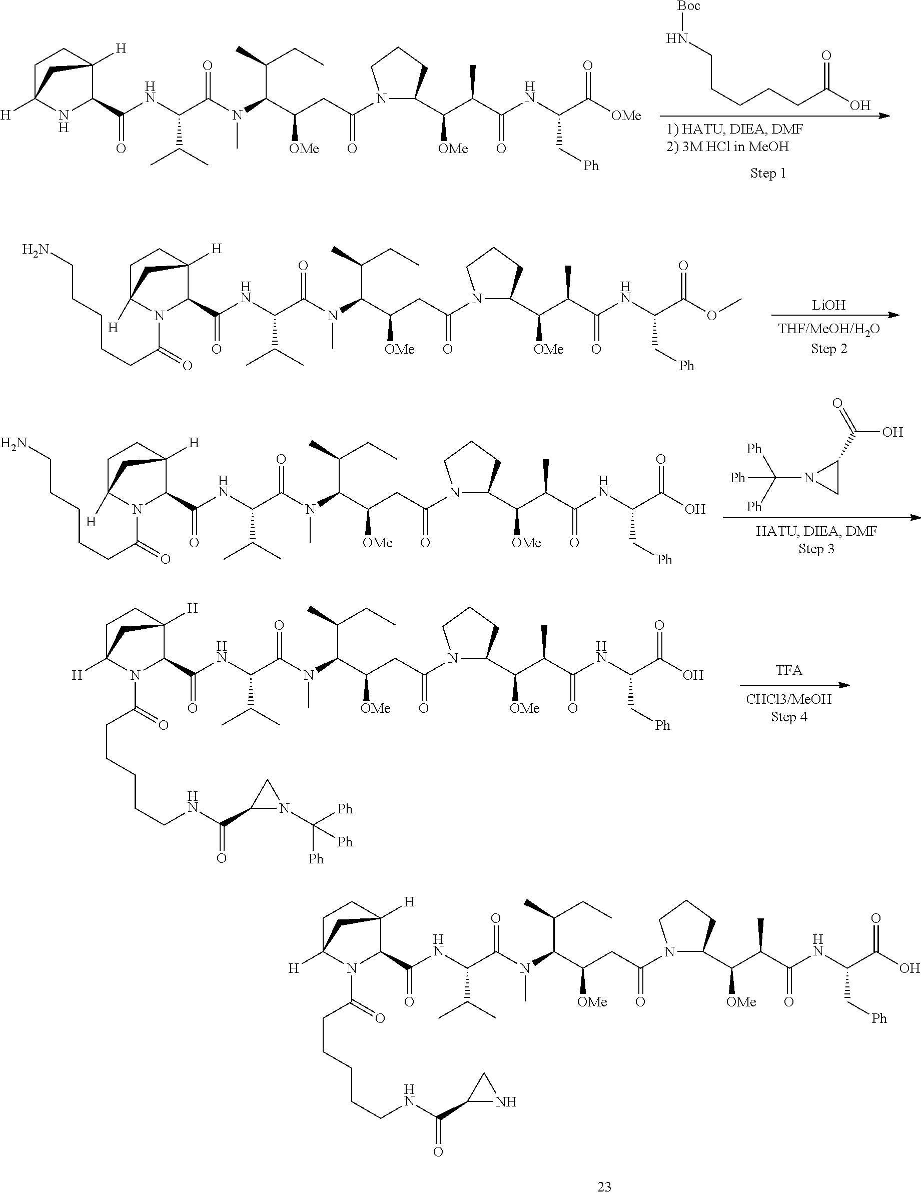

Step 1: In a 7 mL vial, 6-((tert-butoxycarbonyl)amino)hexanoic acid (16 mg, 0.069 mmol) was dissolved in anhydrous DMF (2 mL). DIEA (0.036 mL, 0.21 mmol) and HATU (24 mg, 0.062 mmol) were added. The reaction mixture was stirred for 10 minutes before (S)-methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate (1, HCl salt, 30 mg, 0.034 mmol) was added. The reaction mixture was stirred for an additional 2 hours at room temperature. LCMS indicated the completion of the reaction. The crude was purified by reverse phase HPLC using C18 column, eluted with 10-90% ACN—H₂O containing 0.05% TFA. The fractions containing the desired product were pooled and lyophilized to obtain (S)-methyl 2-((2R,3R)-3-((S)-1-((3R,4S,5S)-4-((S)-2-((1R,3S,4S)-2-(6-((tert-butoxycarbonyl)amino)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate. MS m/z 969.6 (M+1). Retention time 1.42 minutes. The product thus obtained (2 lmg, 0.022 mmol) was dissolved in HCl in MeOH (3M, 2 mL). The solvent was removed slowly under reduced pressure. LCMS analysis of the residue indicated the complete removal of the Boc group. The residue was taken up in acetonitrile and water and lyophilized to give (S)-methyl 2-((2R,3R)-3-((S)-1-((3R,4S,5S)-4-((S)-2-((1R,3S,4S)-2-(6-aminohexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate as HCl salt. MS m/z 869.5 (M+1). Retention time 1.01 minutes.

Step 2: The product from the previous step (10 mg, 0.012 mmol) was dissolved in THF (0.8 mL), MeOH (0.1 mL) and H₂O (0.1 mL). Lithium hydroxide monohydrate (4.83 mg, 0.115 mmol) was added. The reaction mixture was stirred for 4 hours at room temperature. LCMS indicated the completion of the reaction. The solvents were removed under reduced pressure. The residue was neutralized using 0.1N hydrochloric acid, taken up in acetonitrile and H₂O, and lyophilized to give (S)-2-((2R,3R)-3-((S)-1-((3R,4S,5S)-4-((S)-2-((1R,3S,4S)-2-(6-aminohexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid containing some LiCl. MS m/z 855.6 (M+1). Retention time 0.98 minutes.

Step 3: In a 7 mL vial (S)-1-tritylaziridine-2-carboxylic acid (7.6 mg, 0.023 mmol) was dissolved in anhydrous DMF (2 mL). DIEA (0.010 mL, 0.021 mmol) and HATU (7.9 mg, 0.021 mmol) were added. The reaction mixture was stirred for 10 minutes before (S)-2-((2R,3R)-3-((S)-1-((3R,4S,5S)-4-((S)-2-((1R,3S,4S)-2-(6-aminohexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (10 mg, 0.012 mmol). The reaction mixture was stirred at room temperature for an additional 2 hours. LCMS indicated the completion of the reaction. The solvent was removed under reduced pressure. The crude was purified by reverse phase ISCO using C18aq column (5.5 g), eluted with 10-100% acetonitrile-H₂O. The fractions containing the desired product were pooled and lyophilized to give (S)-2-((2R,3R)-3-((S)-1-((3R,4S,5S)-4-(S)—N,3-dimethyl-2-((1R,3S,4S)-2-(6-((S)-1-tritylaziridine-2-carboxamido)hexanoyl)-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoic acid. MS m/z 1166.5 (M+1). Retention time 1.49 minutes.

Step 4: The product from Step 3 (4.0 mg, 0.0034 mmol) was dissolved in MeOH/CHCl₃ (1:1, 1 mL) and cooled to 0° C. TFA (0.0040 mL, 0.051 mmol) was added dropwise. The reaction mixture was warmed to room temperature and stirred for 1 hour. LCMS indicated that the reaction was approximately 60% completed. TFA (0.0040 mL, 0.051 mmol) was added again. After another 1 hour at room temperature LCMS indicated the reaction was complete. The solvents were evaporated under reduced pressure. The residue was dissolved in MeOH and purified by reverse phase ISCO using C18aq column (5.5 g), eluted with 10-100% acetonitrile-H₂O. The fractions containing the desired product were pooled and lyophilized to give (S)-2-((2R,3R)-3-((S)-1-((3R,4S,5S)-4-((S)-2-((1R,3S,4S)-2-(6-((S)-aziridine-2-carboxamido)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (23). MS m/z 924.6 (M+1). Retention time 1.012 minutes.

### Example 3-47

### S)-2-((2R,3R)-3-((S)-1-((3R,4S,5S)-4-((S)-2-((1R,3S,4S)-2-(4-(4-((2,5-Dioxo-2,5-dihydro-1H-pyrrol-1-yl)methyl)-1H-1,2,3-triazol-1-yl)butanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (24)

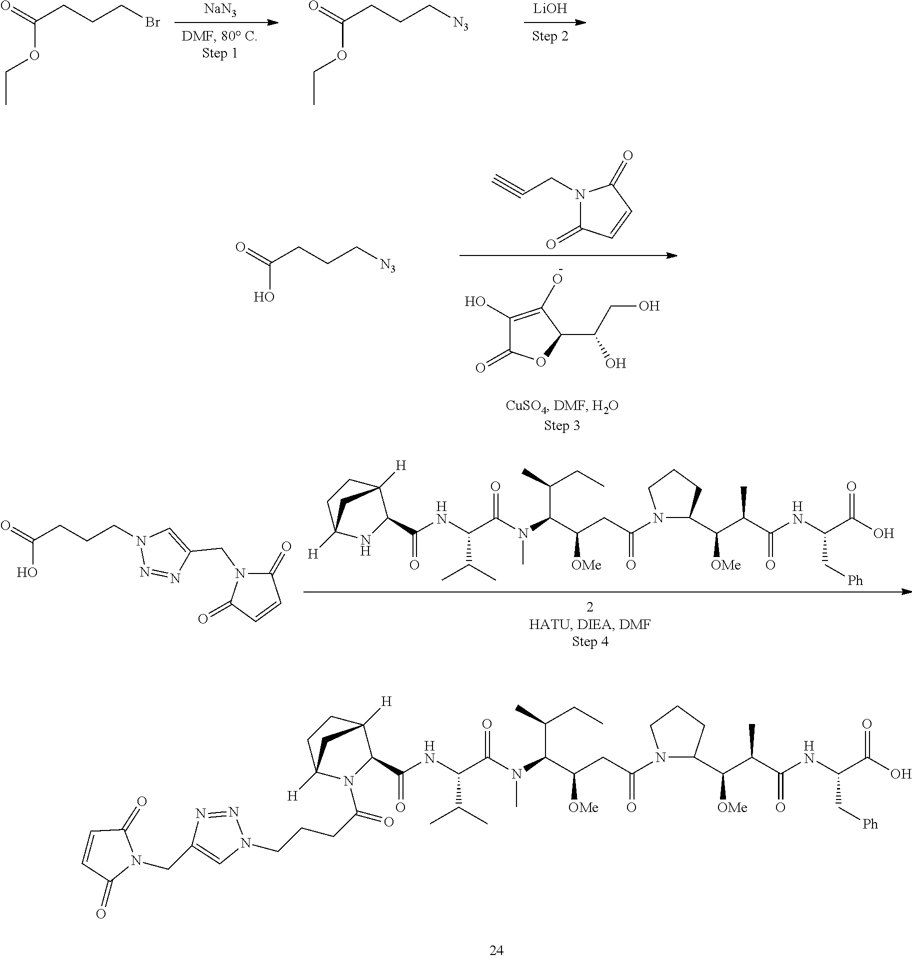

Step 1: To a solution of ethyl 4-bromobutanoate (3.4 g, 0.0174 mol) in DMF (100 mL) was added sodium azide (1.7 g, 0.0262 mol). The mixtute was heated to 80° C. and stirred overnight. The reaction mixture was diluted with water and extracted 3 times with ether. The organic phase was washed with water 3 times, dried over MgSO4, filtered and concentrated to give crude product which was used directly in next step without further purification.

Step 2: Ethyl 4-azidobutanoate (157 mg, 1.0 mmol) was dissolved in THF (4 mL), MeOH (0.5 mL) and water (0.5 mL). Then LiOH.H₂O (168 mg, 4.0 mmol) was added and the reaction mixture was stirred for 2 hours at room temperature. LCMS indicated the completion of the reaction. The reaction was stopped, the pH was adjusted to 2-3 by using 1N HCl and the reaction mixture was extracted with EtOAc. The combined organic phase was dried over MgSO4, concentrated to give crude product which was used directly in next step without further purification. ¹H NMR (400 MHz, CD₃OD): δ 3.36 (t, J=6.8 Hz, 2H), 2.39 (t, J=7.2 Hz, 2H), 1.89-1.82 (m, 2H).

Step 3: A solution of 4-azidobutanoic acid (19 mg, 0.147 mmol), 1-(prop-2-yn-1-yl)-1H-pyrrole-2,5-dione (39.8 mg, 0.294 mmol) and CuSO4 (17.62 mg, 0.11 mmol) in DMF (3.0 mL) and H₂O (0.75 mL) was treated with L-Ascorbic acid sodium salt (72.9 mg, 0.368 mmol) and stirred at room temperature for 2 hours. The reaction mixture was purified by Prep-HPLC, C18 column, eluted with 20-70% acetonitrile-H₂O containing 0.05% TFA. The fractions containing the desired product were pooled and lyophilized to obtain a white solid. MS m/z 265.1 (M+1). Retention time 0.642 minutes. ¹H NMR (400 MHz, CD₃OD): δ 7.94 (s, 1H), 6.86 (s, 2H), 4.77 (s, 2H), 4.43 (t, J=7.0 Hz, 2H), 2.31 (t, J=7.2 Hz, 2H), 2.17-2.13 (m, 2H).

Step 4: 4-(4-((2,5-Dioxo-2,5-dihydro-1H-pyrrol-1-yl)methyl)-1H-1,2,3-triazol-1-yl)butanoic acid (4.5 mg, 0.017 mmol) was dissolved in DMF (1 mL). DIEA (9.9 uL, 0.057 mmol) and HATU (5.61 mg, 0.015 mmol) were added and the mixture was stirred for 10 minutes before the addition of 2 (9.72 mg, 0.011 mmol). The reaction mixture was then stirred for 1 hour at room temperature. LC/MS analysis indicated the completion of the reaction. The product was purified by Prep-HPLC, C18 column, eluted with 20-70% acetonitrile-H₂O containing 0.05% TFA. The fractions containing the desired product 24 were pooled and lyophilized to obtain a white solid. MS m/z 988.5.1 (M+1). Retention time 1.074 minutes.

### Example 3-48

### (1R,3S,4S)—N—((S)-1-(((3R,4S,5S)-1-((S)-2-((1R,2R)-3-(((S)—N-1-(Methylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(6-(2,5-dioxo-2,5-dihydro-1H-pyrrol-1-yl)hexanoyl)-2-azabicyclo[2.2.1]heptane-3-carboxamide (25)

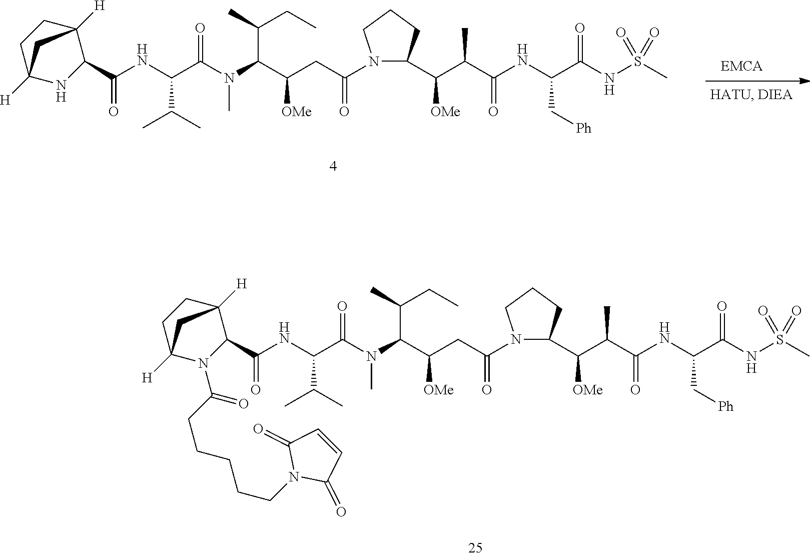 [SMILES: CC[C@H](C)[C@@H]([C@@H](CC(=O)N1CCC[C@H]1[C@H](OC)[C@@H](C)C(=O)N[C@@H](Cc1ccccc1)C(=O)NS(C)(=O)=O)OC)N(C)C(=O)[C@@H](NC(=O)[C@H]1N[C@@H]2CC[C@H]1C2)C(C)C ; CC[C@H](C)[C@@H]([C@@H](CC(=O)N1CCC[C@H]1[C@H](OC)[C@@H](C)C(=O)N[C@@H](Cc1ccccc1)C(=O)NS(C)(=O)=O)OC)N(C)C(=O)[C@@H](NC(=O)[C@@H]1[C@H]2CC[C@H](C2)N1C(=O)CCCCCN1C(=O)C=CC1=O)C(C)C]

EMCA (4.1 mg, 0.019 mmol) was dissolved in DMF (2 mL). DIEA (8.31 mg, 0.064 mmol) and HATU (5.87 mg, 0.015 mmol) were added and after 10 minutes compound 4 (11 mg, 0.013 mmol) was added. The reaction mixture was stirred for 2 hours at room temperature. LC/MS analysis indicated the completion of the reaction. The product was purified by Prep-HPLC, C18 column, eluted with 30-50% acetonitrile-H₂O containing 0.05% TFA. The fractions containing the desired product were pooled and lyophilized to obtain desired product 25 as a white solid. MS m/z 1012.5 (M+1). Retention time 1.222 minutes.

### Example 3-49

### (1R,3S,4S)-2-(6-(2,5-Dioxo-2,5-dihydro-1H-pyrrol-1-yl)hexanoyl)-N—((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (26)

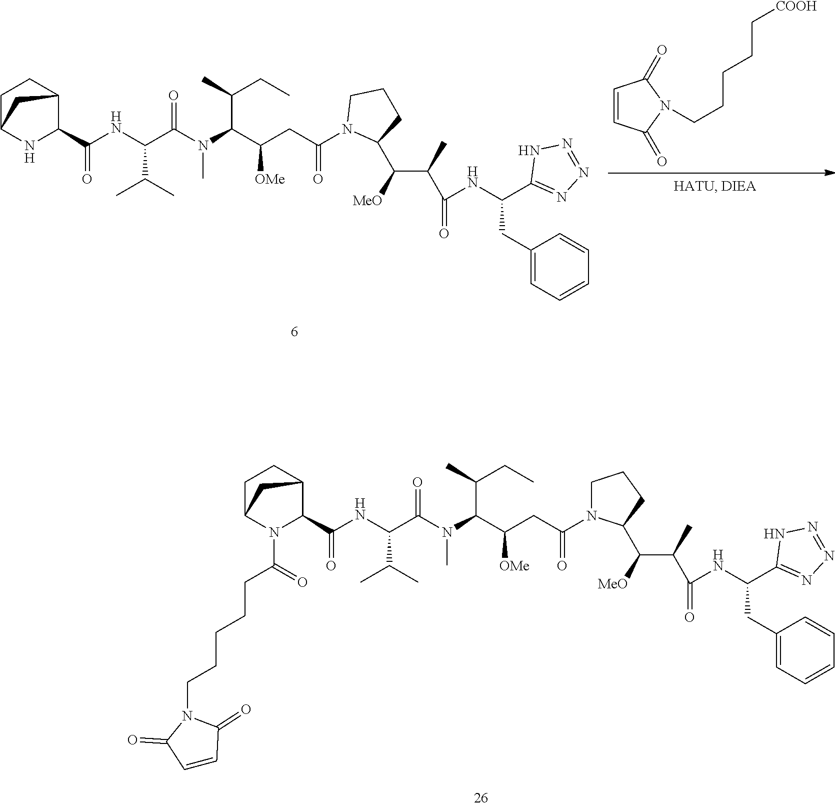 [SMILES: CC[C@H](C)[C@@H]([C@@H](CC(=O)N1CCC[C@H]1[C@H](OC)[C@@H](C)C(=O)N[C@@H](Cc1ccccc1)c1nnn[nH]1)OC)N(C)C(=O)[C@@H](NC(=O)[C@H]1N[C@@H]2CC[C@H]1C2)C(C)C ; O=C(O)CCCCCN1C(=O)C=CC1=O ; CC[C@H](C)[C@@H]([C@@H](CC(=O)N1CCC[C@H]1[C@H](OC)[C@@H](C)C(=O)N[C@@H](Cc1ccccc1)c1nnn[nH]1)OC)N(C)C(=O)[C@@H](NC(=O)[C@@H]1[C@H]2CC[C@H](C2)N1C(=O)CCCCCN1C(=O)C=CC1=O)C(C)C]

To a solution of EMCA (2.5 mg, 0.012 mmol) in DMF (1 ml) was added DIEA (6.2 ul, 0.035 mmol) and then HATU (4.5 mg, 0.012 mmol). The reaction mixture was stirred for 5 minutes and then added to (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(1H-tetrazol-5-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide TFA salt (6.7 mg, 0.0076 mmol). The reaction mixture was kept at room temperature for 1 hour and the crude was purified by reverse phase HPLC, C18 column, eluted with 20-60% acetonitrile-H₂O, containing 0.05% TFA. The fractions containing desired product were concentrated to obtain compound 26 MS m/z 959.5 (M+1). Retention time 1.220 minutes.

### Example 3-50

### ((R)-1-((2R,3R)-3-((S)-1-((3R,4S,5S)-4-((S)-2-((1R,3S,4S)-2-(6-(2,5-Dioxo-2,5-dihydro-1H-pyrrol-1-yl)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid (27)

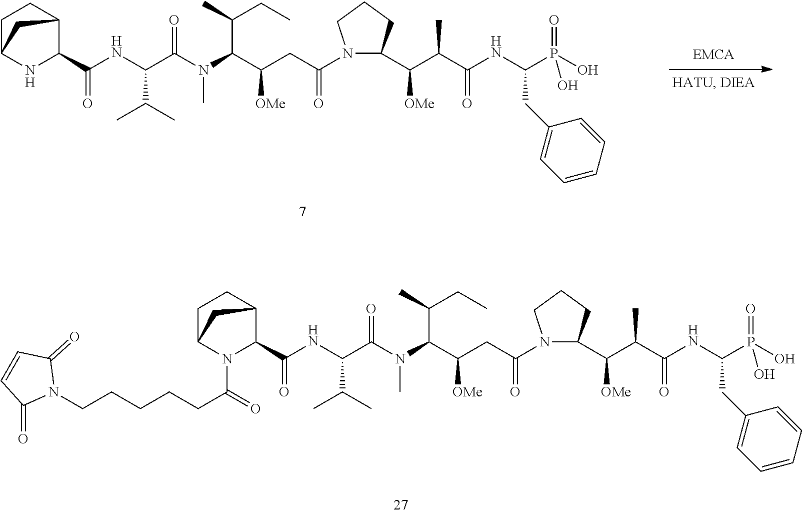 [SMILES: CC[C@H](C)[C@@H]([C@@H](CC(=O)N1CCC[C@H]1[C@H](OC)[C@@H](C)C(=O)N[C@@H](Cc1ccccc1)P(=O)(O)O)OC)N(C)C(=O)[C@@H](NC(=O)[C@H]1N[C@@H]2CC[C@H]1C2)C(C)C ; CC[C@H](C)[C@@H]([C@@H](CC(=O)N1CCC[C@H]1[C@H](OC)[C@@H](C)C(=O)N[C@@H](Cc1ccccc1)P(=O)(O)O)OC)N(C)C(=O)[C@@H](NC(=O)[C@@H]1[C@H]2CC[C@H](C2)N1C(=O)CCCCCN1C(=O)C=CC1=O)C(C)C]

To a solution of EMCA (3.3 mg, 0.016 mmol) in DMF (1 ml) was added DIEA (2.7 ul, 0.016 mmol) and then HATU (5.93 mg, 0.016 mmol). The reaction mixture was stirred at room temperature for 10 minutes and then added to a solution of ((R)-1-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid 7 (10 mg, 0.011 mmol) in DMF. The reaction mixture was stirred at room temperature for 1 hour. The crude was purified by reverse phase HPLC, C18 column, eluted with 30-60% acetonitrile-H₂O, containing 0.05% TFA. The fractions containing desired product were concentrated to ((R)-1-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonic acid 27. MS m/z 971.5 (M+1). Retention time 1.181 minutes.

### Example 3-51

### ((R)-1-((2R,3R)-3-((S)-1-((3R,4S,5S)-4-((S)-2-((1R,3S,4S)-2-(6-(2,5-Dioxo-2,5-dihydro-1H-pyrrol-1-yl)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid (28)

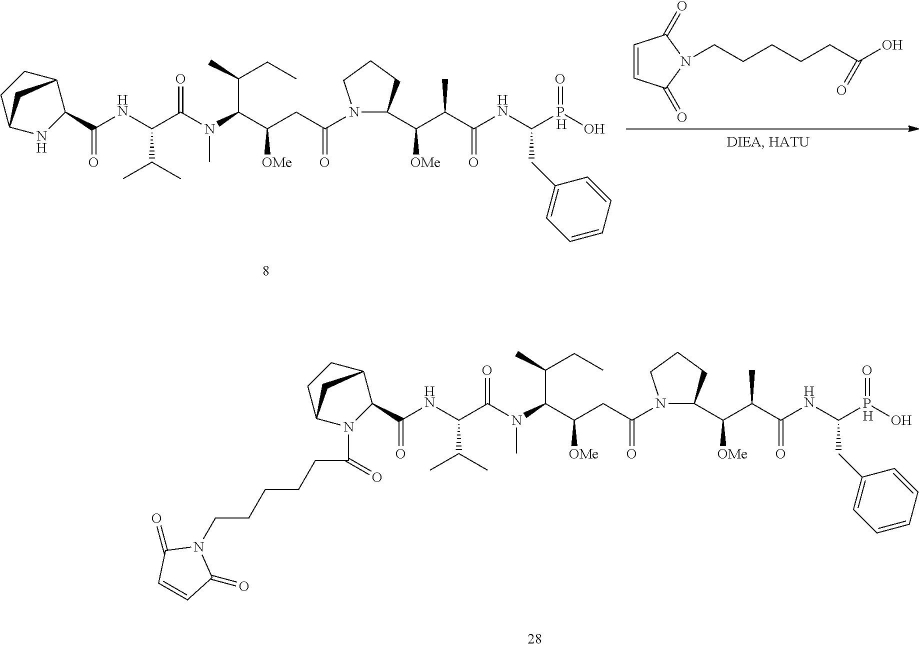

To a solution of EMCA (2.4 mg, 0.011 mmol) in DMF (1 ml) was added DIEA (6.6 ul, 0.038 mmol) and HATU (4.0 mg, 10.42 μmol). The reaction mixture was stirred for 5 minutes and then added to a solution of ((R)-1-(2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid 8 (8.3 mg, 0.0095 mmol) in DMF (1 ml). The reaction mixture was complete in 10 minutes and the crude was purified by reverse phase HPLC, C18 column, eluted with 30-55% acetonitrile-H₂O, containing 0.05% TFA. The fractions containing desired product were concentrated to obtain ((R)-1-(2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid 28. MS m/z 955.5 (M+1). Retention time 1.151 minutes.

### Example 3-52

### Butyl hydrogen ((R)-1-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonate (29)

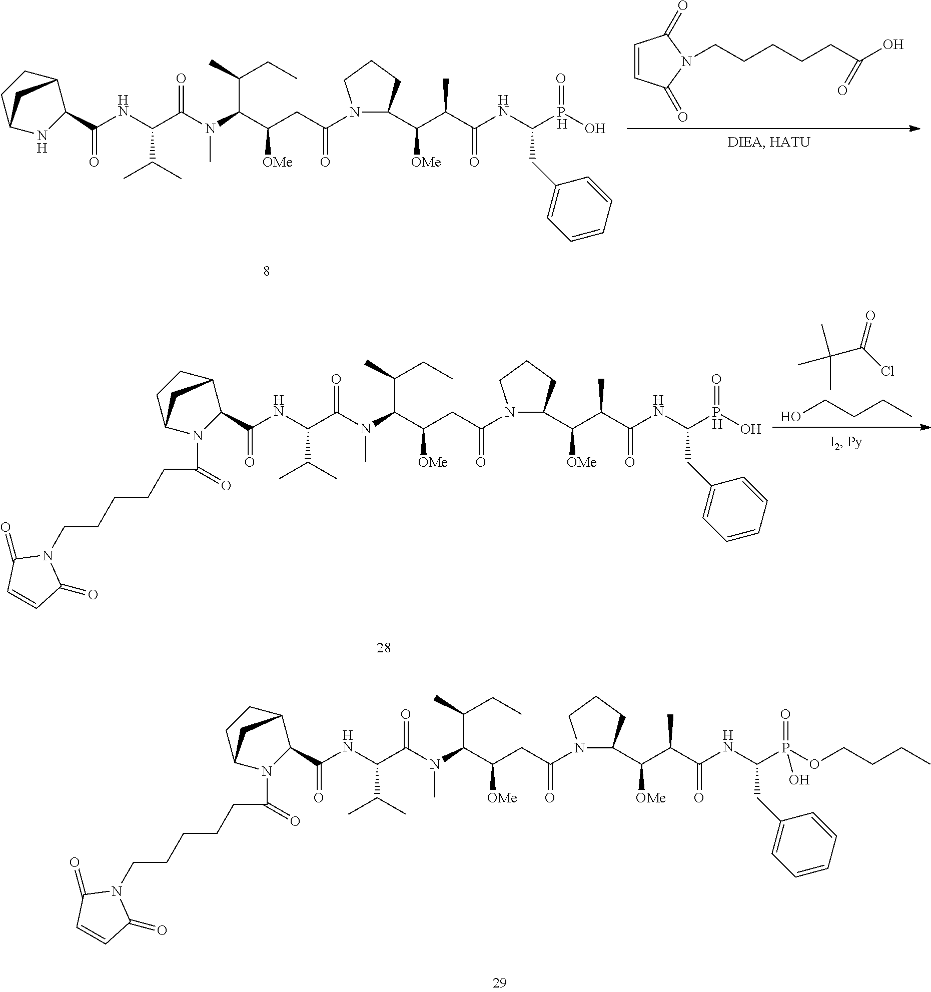

To a solution of ((R)-1-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphinic acid 8 (4.2 mg, 0.0044 mmol) in pyridine (1 ml) was added n-BuOH (3.3 mg, 0.044 mmol) and then pivaloyl chloride (5.3 mg, 0.044 mmol). The reaction was monitored by LCMS until all of the phosphorous acid was converted to the ester. Then a freshly prepared iodine (11 mg, 0.044 mmol) solution in wet pyridine-water (10:1 1 ml) was added. The reaction was monitored by LCMS until completion. Pyridine was removed by vacuum and the crude was purified by reverse phase HPLC, C18 column, eluted with 30-55% acetonitrile-H₂O, containing 0.05% TFA. The fractions containing desired product were concentrated to obtain butyl hydrogen ((R)-1-((2R,3R)-3-((S)-1-((3R,4S,5S)-4-((S)-2-((1R,3S,4S)-2-(6-(2,5-dioxo-2,5-dihydro-1H-pyrrol-1-yl)hexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-2-phenylethyl)phosphonate 29. MS m/z 1027.5 (M+1). Retention time 1.300 minutes. The ester is prone to hydrolysis in acidic condition.

### Example 3-53

### (S)-2-((2R,3R)-3-((S)-1-((3R,4S,5S)-4-((S)-2-((1R,3S,4S)-2-(6-((((1R,8S,9s)-Bicyclo[6.1.0]non-4-yn-9-ylmethoxy)carbonyl)amino)hexanoyl)-2-azabicyclo[2.2.1]heptane-3-carboxamido)-N,3-dim ethylbutanamido)-3-methoxy-5-methylheptanoyl)pyrrolidin-2-yl)-3-methoxy-2-methylpropanamido)-3-phenylpropanoic acid (75)

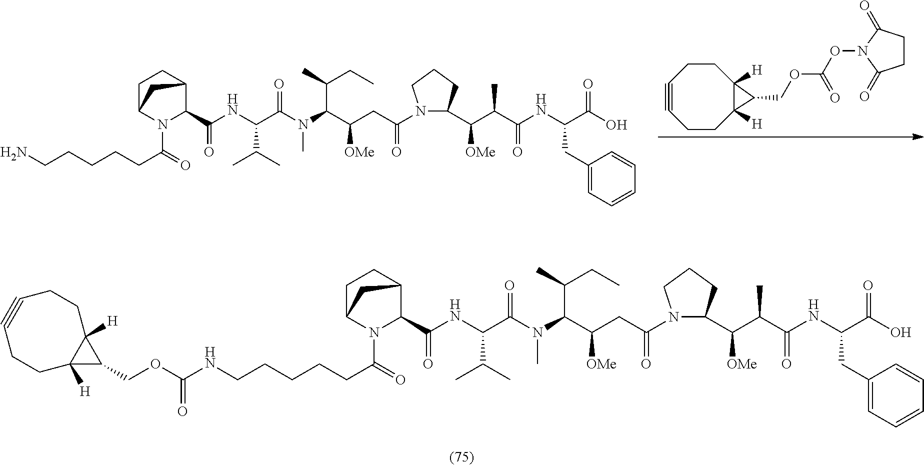 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCCCCN)C(C)C ; O=C(OC[C@@H]1[C@@H]2CCC#CCC[C@@H]21)ON1C(=O)CCC1=O ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCCCCNC(=O)OC[C@@H]1[C@@H]2CCC#CCC[C@@H]21)C(C)C]

To (S)-2-((2R,3R)-3-((S)-1-((3R,4S,5S)-4-((S)-2-((1R,3S,4S)-2-(6-aminohexanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (Step 2, Example 3-44) (5 mg, 0.005 mmol) in DMF-THF (1:1, 2 ml) was added (1R,8S,9s)-bicyclo[6.1.0]non-4-yn-9-ylmethyl (2,5-dioxopyrrolidin-1-yl) carbonate (1.5 mg, 0.005 mmol) and DIEA (0.0025 ml, 0.014 mmol). The reaction mixture was stirred at rt for 30 min and then purified by preparative HPLC (40-65% acetonitrile-H₂O containing 0.05% TFA) to obtain compound (75). MS m/z 1031.6 (M+H). Retention time 1.337 min.

### Example 3-54

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-1-((S)-2-((1R,2R)-3-(((S)-1-Hydroxy-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate (76)

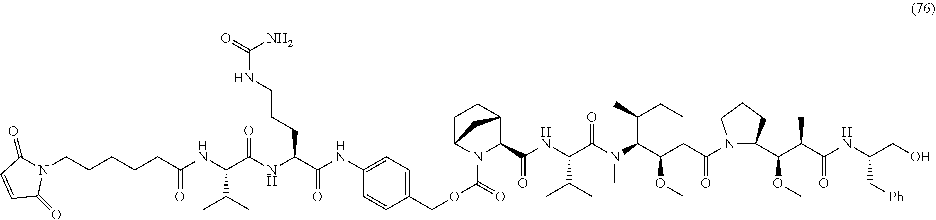 [SMILES: CC[C@H](C)[C@@H]([C@@H](CC(=O)N1CCC[C@H]1[C@H](OC)[C@@H](C)C(=O)N[C@H](CO)Cc1ccccc1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

To a solution of MC-Val-Cit-PAB-PNP (1.9 mg, 0.0026 mmol), compound (50) TFA salt (1.8 mg, 0.002 mmol) in DMF (1 ml) were added pyridine (0.25 ml), HOAT (0.29 mg, 0.002 mmol) and DIEA (0.0054 ml, 0.031 mmol). The reaction was stirred at 40° C. for 2 h and then at 30° C. for 18 h. The reaction mixture was concentrated and purified by preparative HPLC (20-60% acetonitrile-H₂O containing 0.05% TFA) to obtain compound (76). MS m/z 664.0 (M/2+H). Retention time 1.165 min.

### Example 3-55

### (S)-2-((2R,3R)-3-((S)-1-((3R,4S,5S)-4-((S)-2-((1R,3S,4S)-2-(3-(2-(2,5-Dioxo-2,5-dihydro-1H-pyrrol-1-yl)ethoxy)propanoyl)-2-azabicyclo[2.1.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (77)

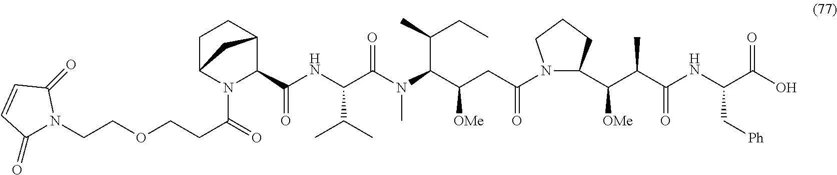 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCOCCN1C(=O)C=CC1=O)C(C)C]

To a solution of 3-(2-(maleimido)ethoxy)propanoic acid (2.2 mg, 0.010 mmol) in DMF (1 ml) were added HATU (3.7 mg, 0.0098 mmol) and DIEA (3.6 mg, 0.028 mmol). The reaction was stirred for 5 min, and then (S)-2-((2R,3R)-3-((S)-1-((3R,4S,5S)-4-((S)-2-((1R,3S,4S)-2-azabicyclo[2.2.1]heptane-3-carboxamido)-N,3-dim ethylbutanamido)-3-methoxy-5-methylheptanoyl)pyrrolidin-2-yl)-3-methoxy-2-methylpropanamido)-3-phenylpropanoic acid (8 mg, 0.0093 mmol) in DMF (0.5 ml) was added. The reaction mixture was stirred at rt for 1 h and then concentrated. The crude was purified by preparative HPLC (10-60% acetonitrile-H₂O containing 0.05% TFA) to obtain compound (77). MS m/z 937.5 (M+H). Retention time 1.138 min.

### Example 3-56

### (S)-2-((2R,3R)-3-((S)-1-((3R,4S,5S)-4-((S)-2-((1R,3S,4S)-2-(3-(2-(2-(2,5-Dioxo-2,5-dihydro-1H-pyrrol-1-yl)ethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (78)

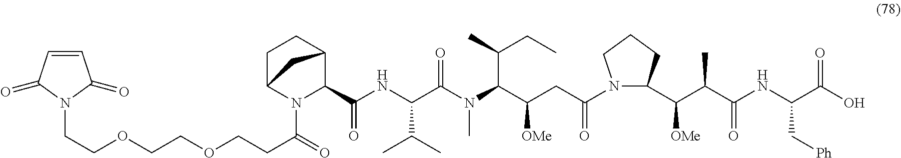 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCOCCOCCN1C(=O)C=CC1=O)C(C)C]

Compound (78) was prepared by the method described for compound (77) using 3-(2-(2-(maleimido)ethoxy)ethoxy)propanoic acid (2.6 mg, 0.010 mmol) in place of 3-(2-(maleimido)ethoxy)propanoic acid. MS m/z 981.5 (M+H). Retention time 1.140 min.

### Example 3-57

### (S)-2-((2R,3R)-3-((S)-1-((3R,4S,5S)-4-((S)-2-((1R,3S,4S)-2-(3-(2-(2-(2-(2,5-Dioxo-2,5-dihydro-1H-pyrrol-1-yl)ethoxy)ethoxy)ethoxy)propanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (79)

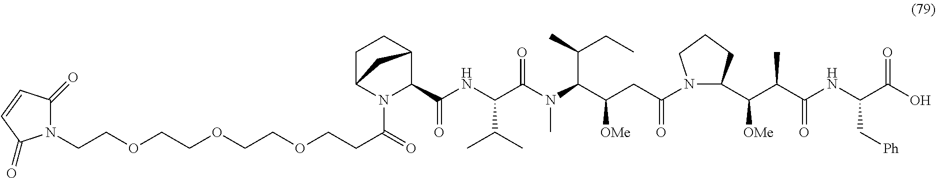 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCOCCOCCOCCN1C(=O)C=CC1=O)C(C)C]

Compound (79) was prepared by the method described for compound (77) using 3-(2-(2-(2-(maleimido)ethoxy)ethoxy)ethoxy)propanoic acid (3.1 mg, 0.010 mmol) in place of 3-(2-(maleimido)ethoxy)propanoic acid. MS m/z 1025.5 (M+H). Retention time 1.143 min.

### Example 3-58

### (S)-2-((2R,3R)-3-((S)-1-((3R,4S,5S)-4-((S)-2-((1R,3S,4S)-2-(1-(2,5-Dioxo-2,5-dihydro-1H-pyrrol-1-yl)-3,6,9,12-tetraoxapentadecan-15-oyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (80)

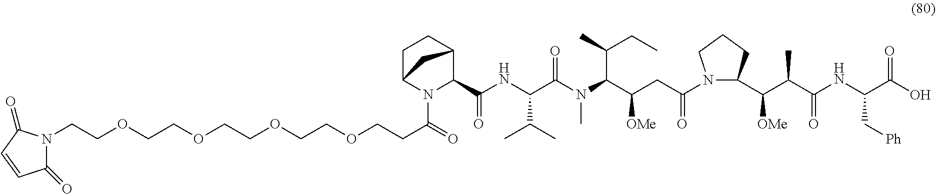 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCOCCOCCOCCOCCN1C(=O)C=CC1=O)C(C)C]

Compound (80) was prepared by the method described for compound (77) using 1-(maleimido)-3,6,9,12-tetraoxapentadecan-15-oic acid (3.6 mg, 0.010 mmol) in place of 3-(2-(maleimido)ethoxy)propanoic acid. MS m/z 1069.5 (M+H). Retention time 1.144 min.

### Example 3-59

### (S)-2-((2R,3R)-3-((S)-1-((3R,4S,5S)-4-((S)—N,3-Dimethyl-2-((1R,3S,4S)-2-(4-(1-(2-(2-(2-(2-(4-(5-(methylsulfonyl)-1,3,4-oxadiazol-2-yl)phenoxy)ethoxy)ethoxy)ethoxy)ethyl)-1H-1,2,3-triazol-4-yl)butanoyl)-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanoic acid (81)

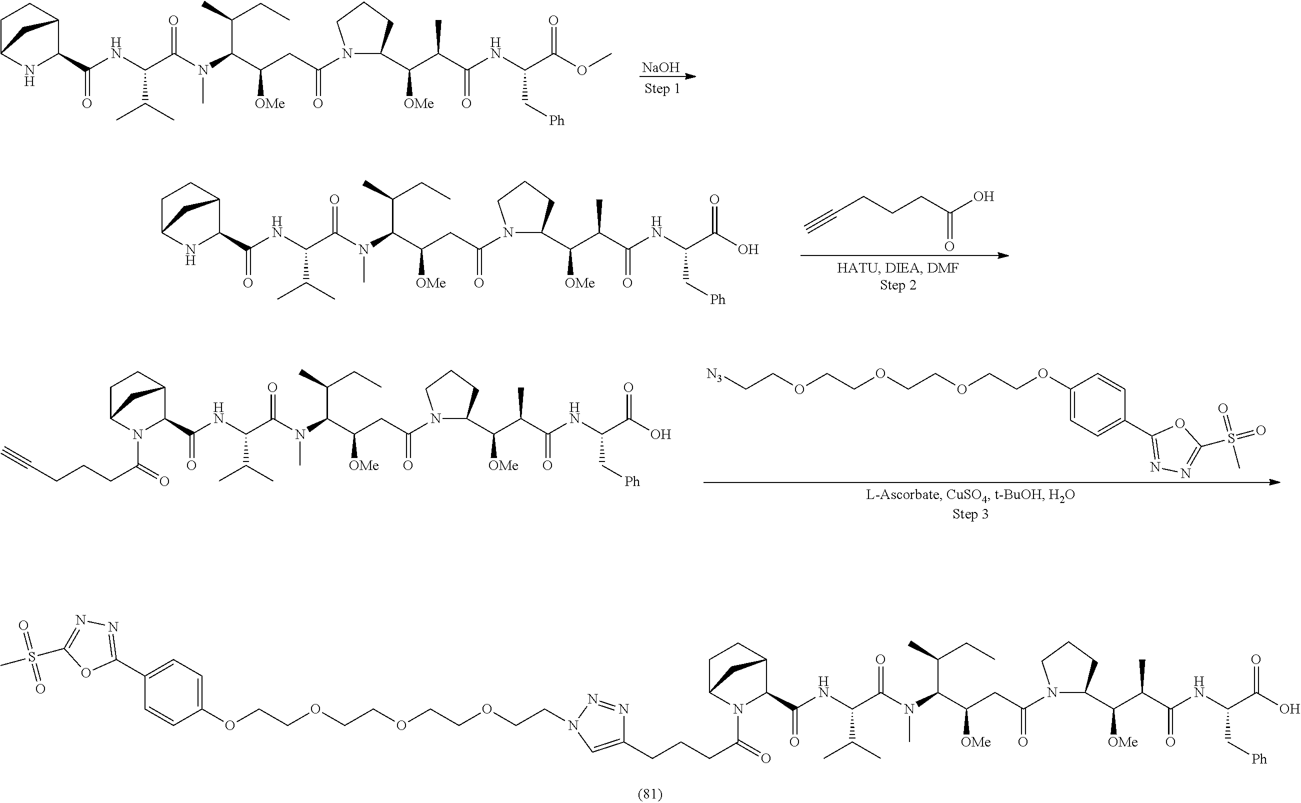

Step 1: (S)-Methyl 2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoate (1) (63 mg, 0.080 mmol) was dissolved in ACN (0.75 ml) and water (0.5 ml). NaOH (1M, 0.35 ml) was added. The reaction was stirred 2 h at rt. After neutralized with 1N HCl to approximately pH 5, the reaction mixture was diluted with water and lyophilized to give crude (S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid. MS m/z 742.4 (M+1). Retention time 1.010 min. The product was used in the next step without further purification.

Step 2: To a solution of hex-5-ynoic acid (5.4 mg, 0.049 mmol) in DMF (2 ml) was added DIEA (26.1 mg, 0.35 mmol) and HATU (16.9 mg, 0.044 mmol). The reaction was stirred at rt for 15 min. Then the product obtained in step 1 (30 mg, 0.040 mmol) was added. The reaction was stirred at rt for 2 h. The crude was purified by preparative HPLC (10-90% acetonitrile-H₂O containing 0.05% TFA) to obtain (S)-2-((2R,3R)-3-((S)-1-((3R,4S,5S)-4-((S)-2-((1R,3S,4S)-2-(hex-5-ynoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid. MS m/z 836.5 (M+1). Retention time 1.224 min.

Step 3: (S)-2-((2R,3R)-3-((S)-1-((3R,4S,5S)-4-((S)-2-((1R,3S,4S)-2-(Hex-5-ynoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (7 mg, 0.0084 mmol) and 2-(4-(2-(2-(2-(2-azidoethoxy)ethoxy)ethoxy)ethoxy)phenyl)-5-(methylsulfonyl)-1,3,4-oxadiazole (3.7 mg, 0.0084 mmol) were suspended in t-BuOH (1 ml) and water (1 ml). Sodium L-ascorbate (1.7 mg, 0.0084 mmol) in 0.3 ml H₂O and CuSO₄ (0.3 mg, 0.0017 mmol) in 0.3 ml H₂O were added sequentially using syringe and the reaction was stirred at rt for 3 h. The reaction mixture was purified by preparative (10-90% acetonitrile-H₂O containing 0.05% TFA) to obtain compound (81) as white solid. MS m/z 639.4 (M/2+1). Retention time 1.196 min.

### Example 3-60

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (82)

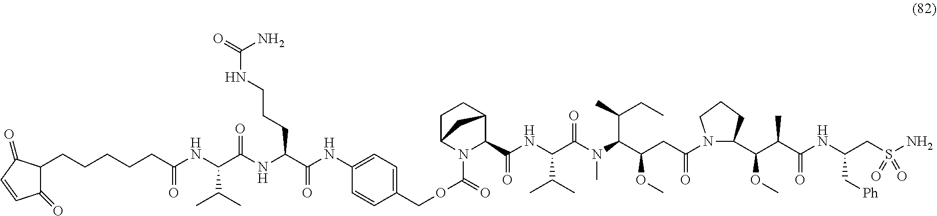 [SMILES: CC[C@H](C)[C@@H]([C@@H](CC(=O)N1CCC[C@H]1[C@H](OC)[C@@H](C)C(=O)N[C@@H](Cc1ccccc1)CS(N)(=O)=O)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCC2C(=O)C=CC2=O)C(C)C)cc1)C(C)C]

(1R,3S,4S)—N—((S)-1-(((3R,4S,5S)-3-Methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)-2-azabicyclo[2.1.1]heptane-3-carboxamide ((56), 8.7 mg, 0.011 mmol), MC-Val-Cit-PABC-PNP (9.7 mg, 0.013 mmol), HOAT (1.7 mg, 0.011 mmol) and DIEA (0.013 ml, 0.077 mmol) were combined in pyridine (0.5 ml) and DMF (2 ml). The reaction was stirred for 4 h at rt. The reaction mixture was purified by preparative HPLC (10-60% acetonitrile-H₂O containing 0.05% TFA) to obtain compound (82) as white solid. MS m/z 695.5 (M12+1). Retention time 1.139 min.

### Example 3-61

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(4-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (83)

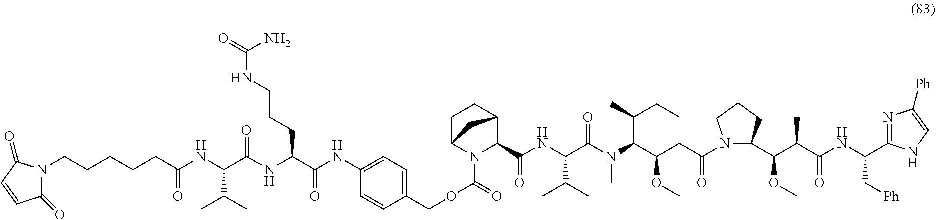 [SMILES: CC[C@H](C)[C@@H]([C@@H](CC(=O)N1CCC[C@H]1[C@H](OC)[C@@H](C)C(=O)N[C@@H](Cc1ccccc1)c1nc(-c2ccccc2)c[nH]1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (83) was prepared by the method described for compound (82) using (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(5-phenyl-1H-imidazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (57), in place of compound (56). MS m/z 720.0 (M/2+1). Retention time 1.169 min.

### Example 3-62

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-3-(((S)-1-methoxy-3-phenylpropan-2-yl)amino)-2-methyl-3-oxopropyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (84)

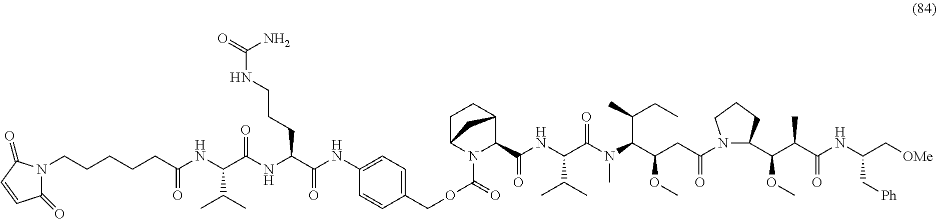 [SMILES: CC[C@H](C)[C@@H]([C@@H](CC(=O)N1CCC[C@H]1[C@H](OC)[C@@H](C)C(=O)N[C@H](COC)Cc1ccccc1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (84) was prepared by the method described for compound (82) using (1R,3S,4S)—N—((S)-1-(((3R,4S,5S)-3-methoxy-1-((S)-2-((1R,2R)-1-methoxy-3-(((S)-1-methoxy-3-phenylpropan-2-yl)amino)-2-methyl-3-oxopropyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (58) in place of compound (56). MS m/z 671.0 (M12+1). Retention time 1.236 min.

### Example 3-63

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(1H-pyrazol-3-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (85)

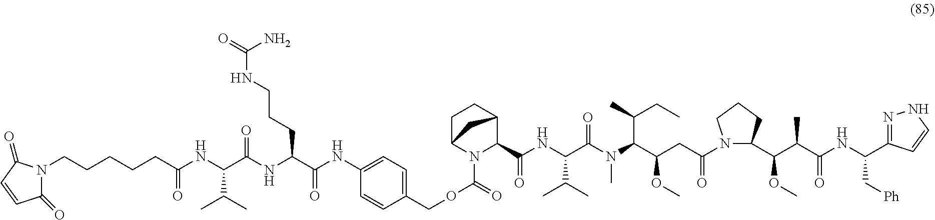 [SMILES: CC[C@H](C)[C@@H]([C@@H](CC(=O)N1CCC[C@H]1[C@H](OC)[C@@H](C)C(=O)N[C@@H](Cc1ccccc1)c1cc[nH]n1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (85) was prepared by the method described for compound (82) using (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(1H-pyrazol-3-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (59) in place of compound (56). MS m/z 682.1 (M/2+1). Retention time 1.172 min.

### Example 3-64

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(5-phenyl-1,3,4-oxadiazol-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (86)

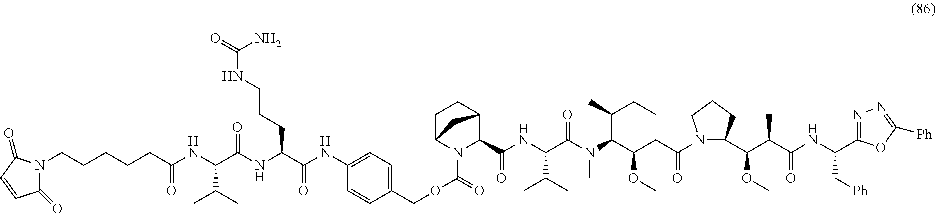 [SMILES: CC[C@H](C)[C@@H]([C@@H](CC(=O)N1CCC[C@H]1[C@H](OC)[C@@H](C)C(=O)N[C@@H](Cc1ccccc1)c1nnc(-c2ccccc2)o1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (86) was prepared by the method described for compound (83) using (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(5-phenyl-1,3,4-oxadiazol-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (62) in place of compound (56). MS m/z 721.1 (M/2+1). Retention time 1.280 min.

### Example 3-65

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((S)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (87)

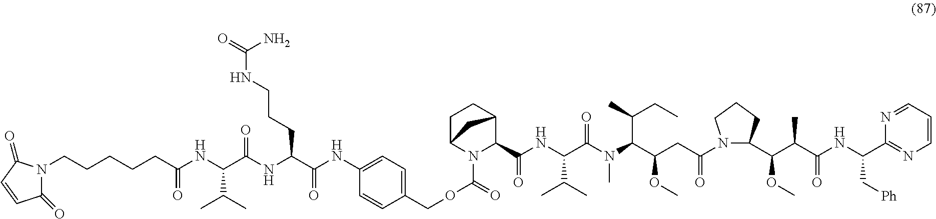 [SMILES: CC[C@H](C)[C@@H]([C@@H](CC(=O)N1CCC[C@H]1[C@H](OC)[C@@H](C)C(=O)N[C@@H](Cc1ccccc1)c1ncccn1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (87) was prepared by the method described for compound (82) using (1R,3S,4S)—N—((S)-1-(((3R,4S,5S)-3-methoxy-1-((S)-2-((1R,2R)-1-methoxy-2-methyl-3-oxo-3-(((S)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (60) in place of compound (56). Retention time 1.204 min.

### Example 3-66

### 4-((S)-2-((S)-2-(6-(2,5-Dioxo-2,5-dihydro-1H-pyrrol-1-yl)hexanamido)-3-methylbutanamido)-5-ureidopentanamido)benzyl (1R,3S,4S)-3-(((S)-1-(((3R,4S,5S)-3-methoxy-1-((S)-2-((1R,2R)-1-methoxy-2-methyl-3-oxo-3-(((R)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (88)

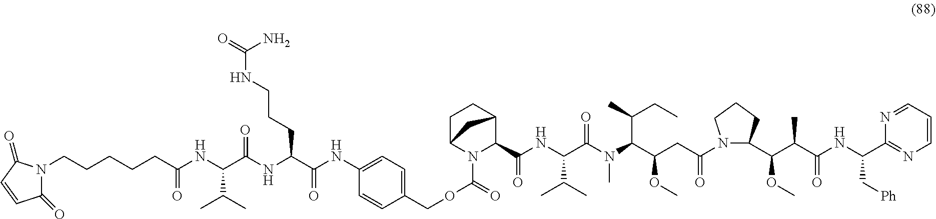 [SMILES: CC[C@H](C)[C@@H]([C@@H](CC(=O)N1CCC[C@H]1[C@H](OC)[C@@H](C)C(=O)N[C@@H](Cc1ccccc1)c1ncccn1)OC)N(C)C(=O)[C@@H](NC(=O)[C@@H]1[C@H]2CC[C@H](C2)N1C(=O)OCc1ccc(NC(=O)[C@H](CCCNC(N)=O)NC(=O)[C@@H](NC(=O)CCCCCN2C(=O)C=CC2=O)C(C)C)cc1)C(C)C]

Compound (88) was prepared by the method described for compound (82) using (1R,3S,4S)—N—((S)-1-(((3R,4S,5S)-3-methoxy-1-((S)-2-((1R,2R)-1-methoxy-2-methyl-3-oxo-3-(((R)-2-phenyl-1-(pyrimidin-2-yl)ethyl)amino)propyl)pyrrolidin-1-yl)-5-methyl-1-oxoheptan-4-yl)(methyl)amino)-3-methyl-1-oxobutan-2-yl)-2-azabicyclo[2.2.1]heptane-3-carboxamide (61) in place of compound (56). MS m/z 688.0 (M/2+1). Retention time 1.221 min.

### Example 3-67

### (S)-2-((2R,3R)-3-((S)-1-((3R,4S,5S)-4-((S)-2-((1R,3S,4S)-2-(6-(5-((2,5-Dioxopyrrolidin-1-yl)oxy)-5-oxopentanamido)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (89)

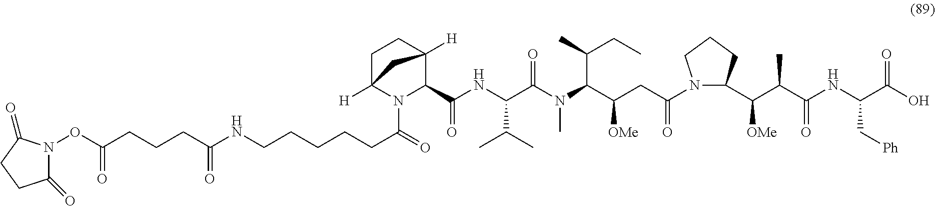 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H]1[C@H]2CC[C@H](C2)N1C(=O)CCCCCNC(=O)CCCC(=O)ON1C(=O)CCC1=O)C(C)C]

A solution of (S)-2-((2R,3R)-3-((S)-1-((3R,4S,5S)-4-((S)-2-((1R,3S,4S)-2-(6-aminohexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid ((Step 2, Example 3-44) 20 mg, 0.021 mmol) and DIEA (0.018 ml, 0.10 mmol) in DMF (1 ml) was added to bis(2,5-dioxopyrrolidin-1-yl) glutarate (10.1 mg, 0.031 mmol) and DIEA (0.018 ml) in DMF (1 ml). The reaction was stirred for 2 h at rt. The crude was purified by preparative HPLC (20-70% acetonitrile-H₂O containing 0.05% TFA) to obtain (S)-2-((2R,3R)-3-((S)-1-((3R,4S,5S)-4-((S)-2-((1R,3S,4S)-2-(6-(5-((2,5-dioxopyrrolidin-1-yl)oxy)-5-oxopentanamido)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (89). MS m/z 1066.5 (M+1). Retention time 1.103 min.

### Synthetic Procedure for Example C-Terminal Linked Compounds

### Example 3-68

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (30)

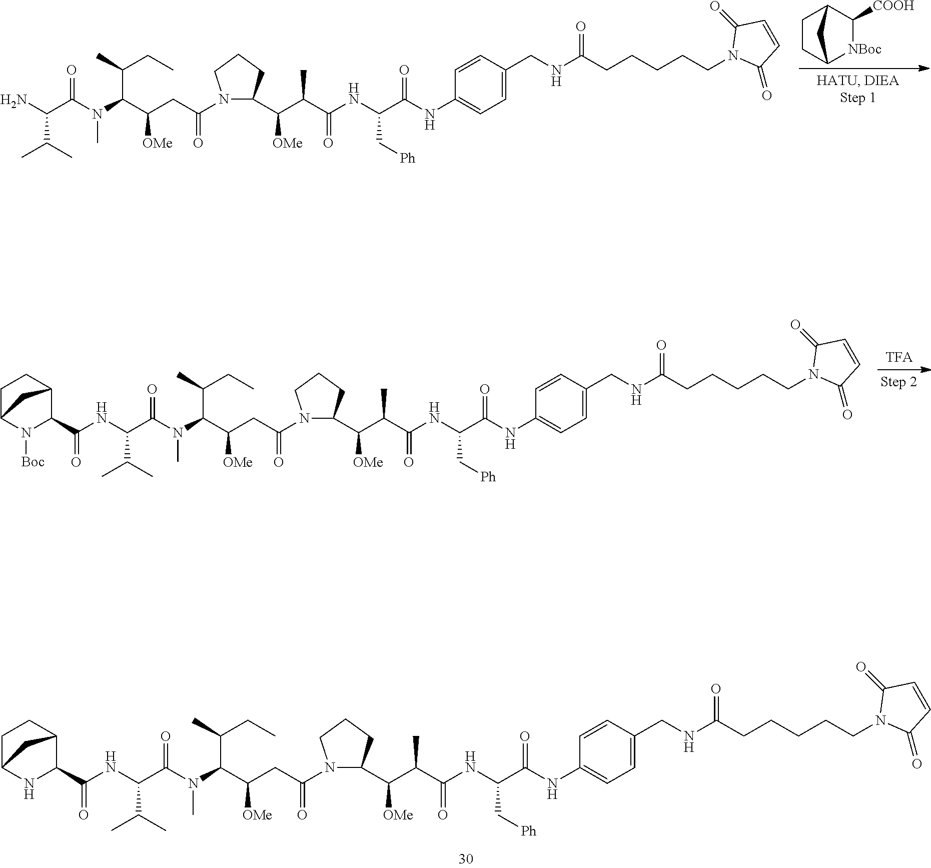

Step 1: In a 15 mL round bottom flask were added (1R,3S,4S)-2-(tert-butoxycarbonyl)-2-azabicyclo[2.2.1]heptane-3-carboxylic acid (12 mg, 0.050 mmol) and DIEA (0.032 mL, 0.18 mmol) in DMF (2.0 mL), followed by HATU (19 mg, 0.050 mmol). The resuting solution was stirred for 5 minutes. Then N-(4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-3-phenylpropanamido)benzyl)-6-(2,5-dioxo-2,5-dihydro-1H-pyrrol-1-yl)hexanamide TFA salt (48.5 mg, 0.047 mmol) was added. The reaction mixture was stirred at room temperature for 1 hour. The crude was purified by reverse phase HPLC, C18 column, eluted with 10-70% acetonitrile-H₂O, containing 0.05% TFA. The fractions containing the desired product were pooled and concentrated to obtain (1R,3S,4S)-tert-butyl 3-(((S)-1-(((3R,4S,5S)-1-((S)-2-((1R,2R)-3-(((S)-1-((4-((6-(2,5-dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate, MS m/z 1139.6 (M+1). Retention time 1.39 minutes.

Step 2: In a 15 mL round bottom flask were added the product obtained in step 1 (42.6 mg, 0.037 mmol), TFA (2.0 mL) and DCM (4.0 mL), resulting in a clear solution. The reaction mixture was stirred at room temperature for 1 hour at which time LCMS a analysis showed Boc was completely removed. The reaction mixture mixture was concentrated to obtain compound 30 as TFA salt, MS m/z 1039.6 (M+1). Retention time 1.06 minutes.

### Example 3-69

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (31)

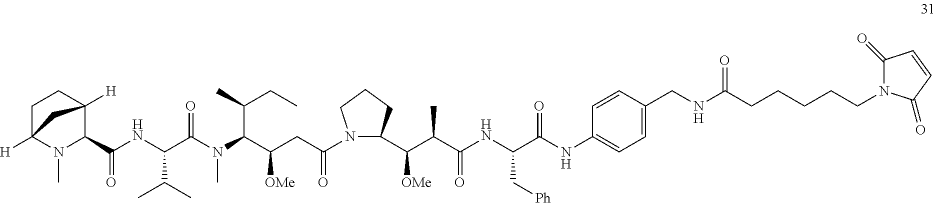 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1[C@H]2CC[C@H](C2)N1C)C(C)C]

In a 15 mL round bottom flask were added (1R,3S,4S)—N—((S)-1-(((3R,4S,5S)-1-((S)-2-((1R,2R)-3-(((S)-1-((4-((6-(2,5-dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide TFA salt (30) (20 mg, 0.017 mmol), paraformaldehyde (5.9 mg, 0.21 mmol), and acetic acid (0.0029 mL, 0.050 mmol) in MeOH (2.0 mL). To the resulting suspension was added NaCNBH₃ (6.6 mg, 0.11 mmol). The reaction mixture was stirred at room temperature for 18 hours. Additional formaldehyde and NaCNBH₃ were added and the reaction mixture was heated to 50° C. for 1 hour to complete the reaction. The crude was purified by reverse phase HPLC, C18 column, eluted with 10-50% acetonitrile-H₂O, containing 0.05% TFA. The fractions containing the desired product were pooled and concentrated to obtain compound 31 as TFA salt, MS m/z 1053.7 (M+1)). Retention time 1.07 minutes.

### Example 3-70

### (1R,3S,4S)-2-Acetyl-N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (32)

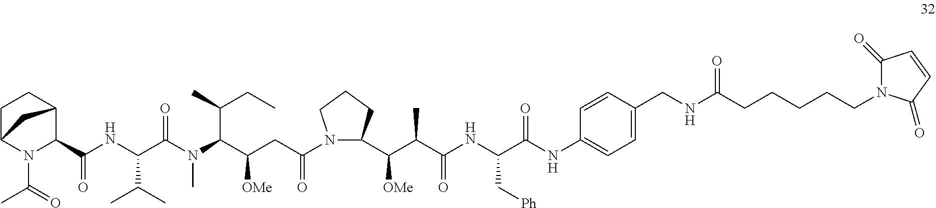 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1[C@H]2CC[C@H](C2)N1C(C)=O)C(C)C]

In a 15 mL round bottom flask were added acetic acid (0.79 mg, 0.013 mmol), DIEA (1.7 mg, 0.013 mmol) and DMF (1.0 mL), followed by HBTU (2.2 mg, 0.0058 mmol). The reaction mixture was stirred for 5 minutes before (2S,3S)—N—((S)-1-(((3R,4S,5S)-1-((S)-2-((1R,2R)-3-(((S)-1-((4-((6-(2,5-dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-3-methylpyrrolidine-2-carboxamide TFA salt (30) (5.5 mg, 0.0048 mmol) was added. The reaction mixture was stirred at room temperature for 1 hour. The crude was purified by reverse phase HPLC, C18 column, eluted with 20-50% acetonitrile-H₂O, containing 0.05% TFA. The fractions containing the desired product were pooled and concentrated to obtain compound 32, MS m/z 1081.3 (M+1). Retention time 1.22 minutes.

### Example 3-71

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(6-hydroxyhexyl)-2-azabicyclo[2.1.1]heptane-3-carboxamide (33)

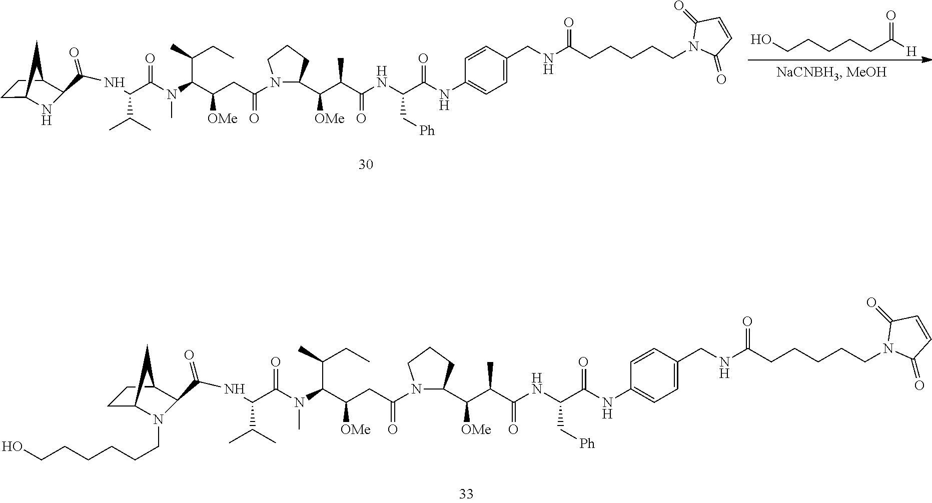

To compound 30 (3.0 mg, 0.0029 mmol) in MeOH (1.0 mL) was added 6-hydroxyhexanal (6.7 mg, 0.058 mmol), followed by NaBH₃CN (9.1 mg, 0.14 mmol). After 30 minutes, additional NaBH₃CN (9.1 mg, 0.14 mmol) was added. After another 30 minutes, LCMS analysis indicated the completion of the reaction. The reaction mixture mixture was purified by reverse phase HPLC using C18 column, eluted with 10-90% acetonitrile-H₂O containing 0.05% TFA. The fractions containing the desired product were pooled and lyophilized to obtain (1R,3S,4S)—N—((S)-1-(((3R,4S,5S)-1-((S)-2-((1R,2R)-3-(((S)-1-((4-((6-(2,5-dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(6-hydroxyhexyl)-2-azabicyclo[2.1.1]heptane-3-carboxamide 33, MS m/z 1139.6 (M+1). Retention time 1.10 minutes.

### Example 3-72

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(6-hydroxyhexanoyl)-2-azabicyclo[2.2.1]heptane-3-carboxamide (34)

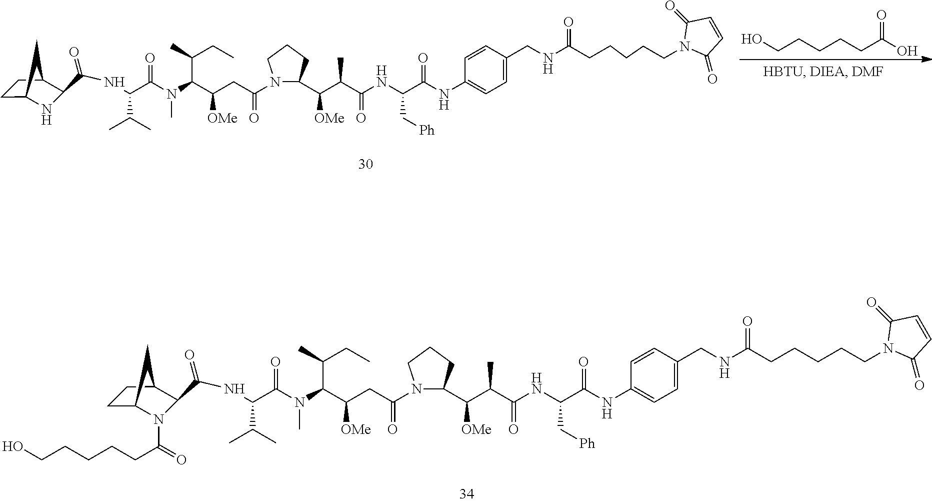

To 6-hydroxyhexanoic acid (3.8 mg, 0.029 mmol) in DMF (1 mL) were added DIEA (7.5 mg, 0.058 mmol) and HBTU (9.1 mg, 0.024 mmol). After 10 minutes, compound 30 (10 mg, 0.0096 mmol) was added. The reaction mixture was stirred for 1 hour, at which time LCMS analysis indicated the completion of the reaction. The reaction mixture was purified by reverse phase HPLC using C18 column, eluted with 10-90% acetonitrile-H₂O containing 0.05% TFA. The fractions containing the desired product were pooled and lyophilized to obtain compound 34, MS m/z 1153.5 (M+1). Retention time 1.20 minutes.

### Example 3-73

### (2S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-3-azabicyclo[3.1.0]hexane-2-carboxamide (35)

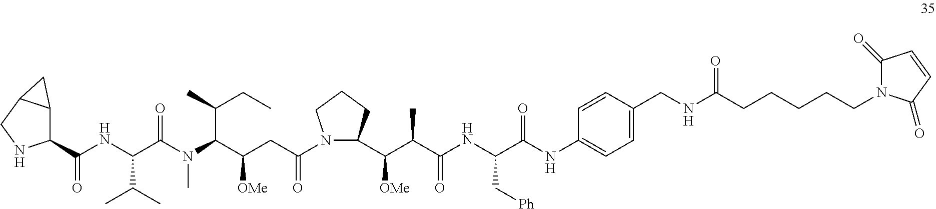 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@H]1NCC2CC21)C(C)C]

This compound was synthesized using the same method as described for compound 30 using 3-(tert-Butoxycarbonyl)-3-azabicyclo[3.1.0]hexane-2-carboxylic acid (12.5 mg, 0.055 mmol), DIEA (28.5 mg, 0.22 mmol), HATU (21 mg, 0.055 mmol) and N-(4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-3-phenylpropanamido)benzyl)-6-(2,5-dioxo-2,5-dihydro-1H-pyrrol-1-yl)hexanamide TFA salt (11 mg, 0.011 mmol). After purification, the Boc-protected intermediate was obtained, MS m/z 1125.5 (M+1). Retention time 1.34 minutes. The product thus obtained (10 mg, 0.0089 mmol) was treated with TFA (2.0 mL) in DCM (4.0 mL). The reaction mixture was stirred at room temperature for 1 hour and LCMS analysis showed Boc was completely removed. The solution was concentrated to obtain compound 35 as TFA salt, MS m/z 1025.5 (M+1). Retention time 1.06 minutes.

### Example 3-74

### (2S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-3-methyl-3-azabicyclop.1.01hexane-2-carboxamide (36)

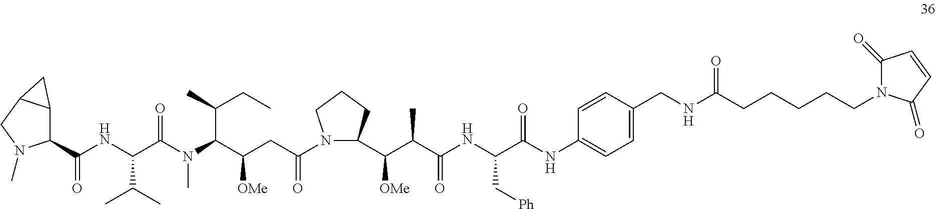 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1C2CC2CN1C)C(C)C]

To (2S)—N—((S)-1-(((3R,4S,5S)-1-((S)-2-((1R,2R)-3-(((S)-1-((4-((6-(2,5-dioxo-2,5-dihydro-1H-pyrrol-1- yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-3-azabicyclo[3.1.0]hexane-2-carboxamide TFA salt (35) (7.9 mg, 0.0062 mmol) and paraformaldehyde (2.7 mg, 0.089 mmol) in MeOH (2.0 mL) was added NaCNBH₃ (11 mg, 0.018 mmol). The reaction mixture was stirred at 50° C. for 2 hours. The crude was purified by reverse phase HPLC, C18 column, eluted with 20-50% acetonitrile-H₂O, containing 0.05% TFA. The fractions containing the desired product were pooled and concentrated to obtain compound 36 as TFA salt, MS m/z 1039.5 (M+1). Retention time 1.07 minutes.

### Example 3-75

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(2,5,8,11,14,17,20,23,26,29,32,35-dodecaoxaoctatriacontan-38-oyl)-2-azabicyclo[2.2.1]heptane-3-carboxamide (37)

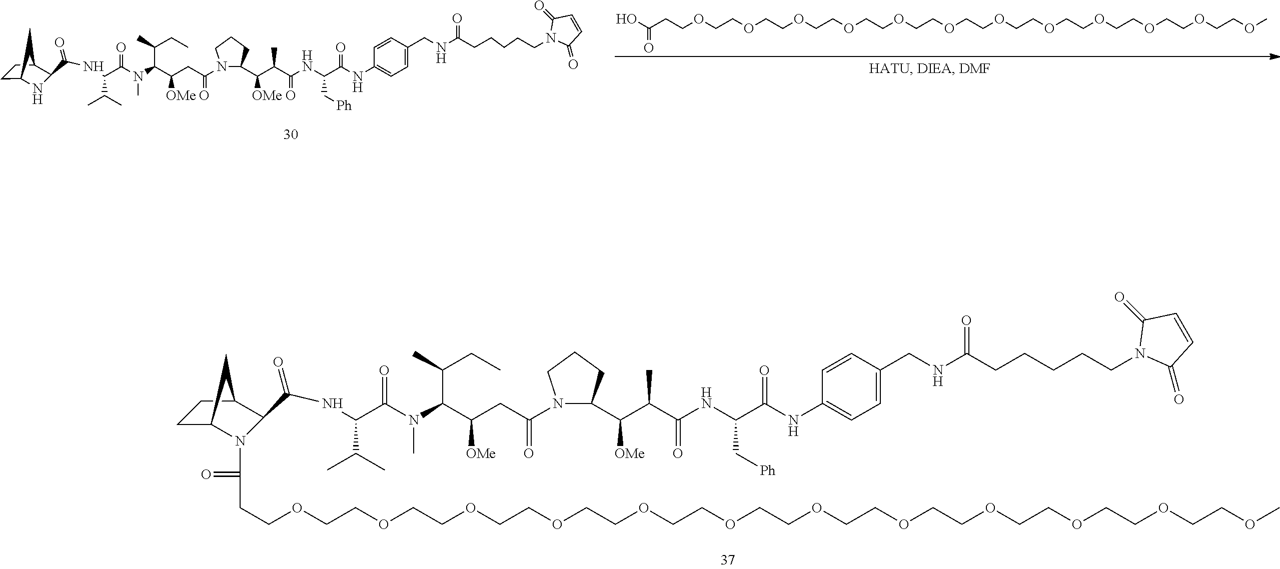 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@H]1N[C@H]2CC[C@H]1C2)C(C)C ; COCCOCCOCCOCCOCCOCCOCCOCCOCCOCCOCCOCCC(=O)O ; CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1[C@H]2CC[C@@H](C2)N1C(=O)CCOCCOCCOCCOCCOCCOCCOCCOCCOCCOCCOCCOC)C(C)C]

To 2,5,8,11,14,17,20,23,26,29,32,35-dodecaoxaoctatriacontan-38-oic acid (17.0 mg, 0.029 mmol) in DMF (1.5 mL) were added DIEA (7.5 mg, 0.058 mmol) and HATU(9.2 mg, 0.024 mmol). After 10 minutes, compound 30 (10.0 mg, 0.0096 mmol) was added. The reaction mixture was stirred for 2 hours at room temperature. The reaction mixture was purified by reverse phase HPLC using C18 column, eluted with 10-90% acetonitrile-H₂O containing 0.05% TFA. The fractions containing the desire product were pooled and lyophilized to obtain compound 37, MS m/z 805.6 ((M+2)/2). Retention time 1.25 minutes.

### Example 3-76

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(2,5,8,11,14,17,20,23-octaoxahexacosan-26-oyl)-2-azabicyclo[2.2.1]heptane-3-carboxamide (38)

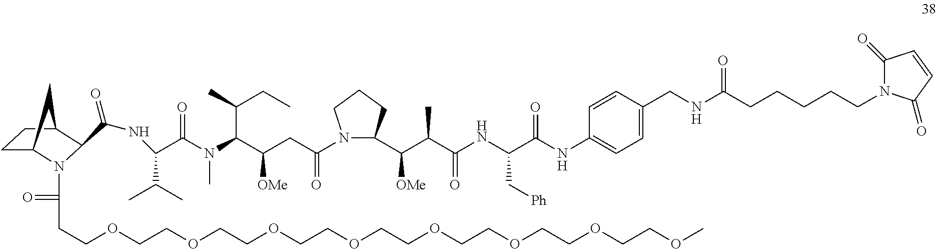 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1[C@H]2CC[C@@H](C2)N1C(=O)CCOCCOCCOCCOCCOCCOCCOCCOC)C(C)C]

Compound 38 was synthesized by the same method as described for compound 37 using compound 30 (10 mg, 0.0096 mmol) and 2,5,8,11,14,17,20,23-octaoxahexacosan-26-oic acid (11.91 mg, 0.029 mmol), MS m/z 717.5 ((M+2)/2). Retention time 1.25 minutes.

### Example 3-77

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(1-hydroxy-3,6,9,12-tetraoxapentadecan-15-oyl)-2-azabicyclo[2.2.1]heptane-3-carboxamide (39)

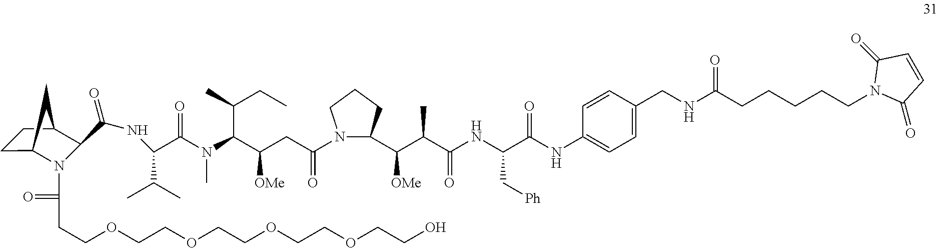 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCCCCN2C(=O)C=CC2=O)cc1)OC)N(C)C(=O)[C@@H](NC(=O)[C@@H]1[C@H]2CC[C@@H](C2)N1C(=O)CCOCCOCCOCCOCCO)C(C)C]

Compound 39 was synthesized by the same method as described for compound 37 using using 30 (10 mg, 0.0096 mmol), 1-hydroxy-3,6,9,12-tetraoxapentadecan-15-oic acid (7.7 mg, 0.029 mmol), DIEA (7.46 mg, 0.058 mmol) and HBTU (9.12 mg, 0.024 mmol) in DMF (1.5 mL), MS m/z 1287.6 (M+1)). Retention time 1.18 minutes.

### Example 3-78

### (1R,3S,4S)—N—((S)-1-(((3R,4S,5S)-1-((S)-2-((1R,2R)-3-(((S)-1-((4-((4-((2,5-Dioxo-2,5-dihydro-1H-pyrrol-1-yl)methyl)cyclohexanec arboxamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (40)

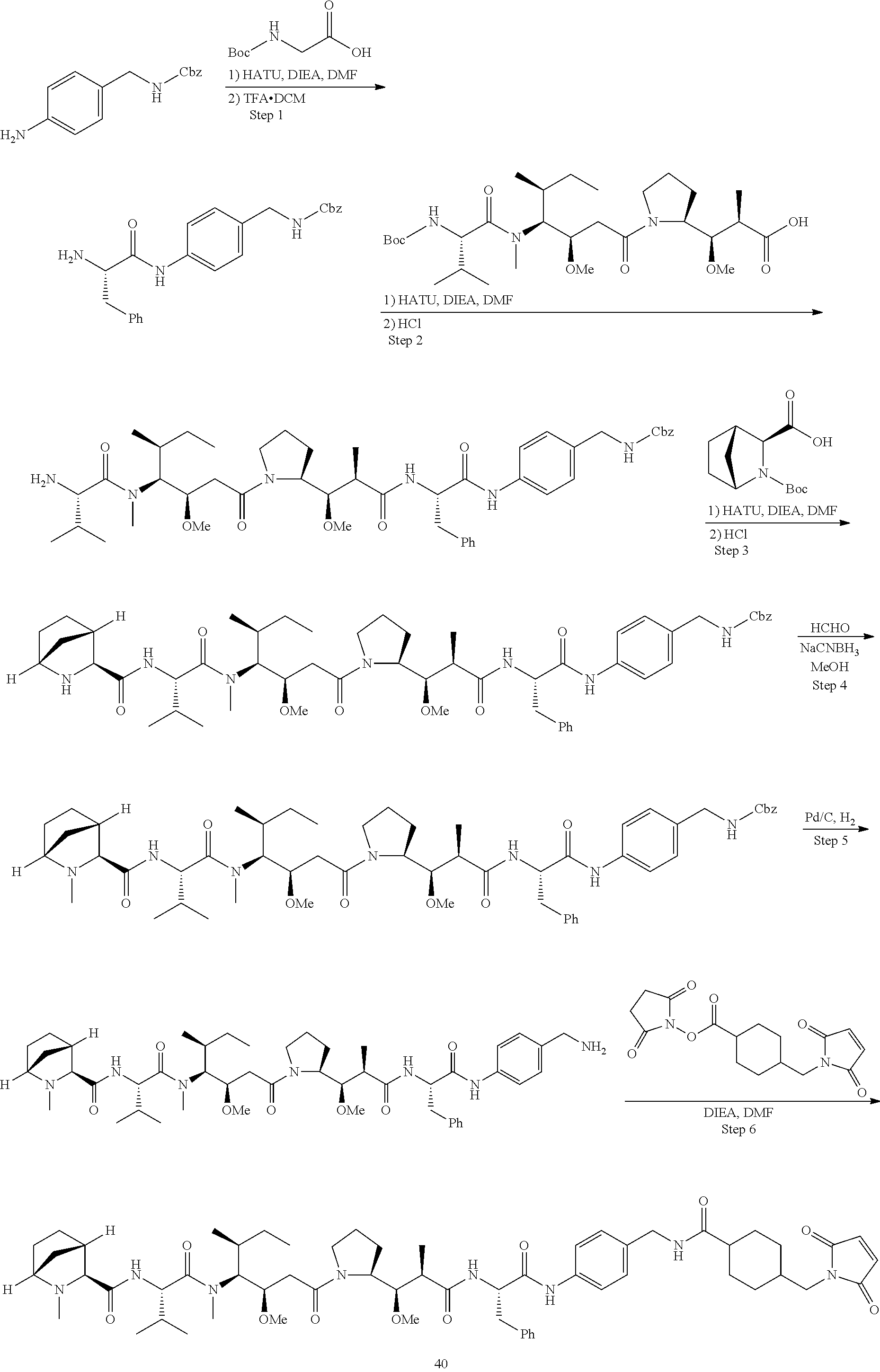

Step 1: (S)-2-((tert-Butoxycarbonyl)amino)-3-phenylpropanoic acid (964 mg, 3.63 mmol) was dissolved in DMF (10 mL). DIEA (1.27 g, 9.84 mmol) and HATU (1.13 g, 3.03 mmol) were added. After 10 minutes, benzyl 4-aminobenzylcarbamate (388 mg, 1.51 mmol) was added. The reaction mixture was stirred for 2 hours at room temperature at which time LCMS analysis indicated the completion of the reaction. EtOAc (60 mL) was added to the reaction. Then the reaction mixture was washed with saturated aqueous NaHCO₃. The aqueous layer was extracted with EtOAc (2×30 mL). The combined organic phases were washed with H₂O (5×10 mL), dried over MgSO₄, filtered and concentrated to afford the crude product. The crude product was dissolved in DCM (5.0 mL) and treated with TFA (5.0 mL). After 1 hour at room temperature, LCMS analysis indicated the completion of the reaction. Solvents were removed under reduced pressure. The residue was purified by ISCO using 0-8% MeOH with 2M ammonia in DCM to obtained (S)-benzyl 4-(2-amino-3-phenylpropanamido)benzylcarbamate as a white solid, MS m/z 404.2(M+1)). ¹H NMR (400 MHz, CD₃OD): δ 7.44-7.23 (m, 14H), 5.10 (s, 2H), 4.26 (s, 2H), 4.12 (d, J=7.4 Hz, 1H), 3.28-3.22 (m, 1H), 3.15-3.10 (m, 1H).

Step 2: (S)-Benzyl 4-(2-amino-3-phenylpropanamido)benzylcarbamate (201.7 mg, 0.50 mmol) and (2R,3R)-3-((S)-1-((3R,4S,5S)-4-((S)-2-((tert-butoxycarbonyl)amino)-N,3-dimethylbutanamido)-3-methoxy-5-methylheptanoyl)pyrrolidin-2-yl)-3-methoxy-2-methylpropanoic acid (429 mg, 0.75 mmol) were dissolved in DMF (6 mL). Then DIEA (323 mg, 2.50 mmol) and HATU (342 mg, 0.90 mmol) were added. The reaction mixture was stirred for 1 hour at room temperature. The reaction mixture was purified by reverse phase HPLC to afford benzyl 4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-3-phenylpropanamido)benzylcarbamate, MS m/z 957.5 (M+1), Retention time 1.54 minutes. The Boc protected product (393 mg, 0.41 mmol) was dissolved in methanolic HCl (3 M, 15 mL). The solvent was slowly evaporated under reduced pressure. LCMS analysis indicated the completion of the deprotection reaction. Acetonitrile and water were added and the resulting solution was lyophilized to obtain benzyl 4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-3-phenylpropanamido)benzylcarbamate as a HCl salt, MS m/z 857.5 (M+1). Retention time 1.16 minutes.

Step 3: (1R,3S,4S)-2-(tert-Butoxycarbonyl)-2-azabicyclo[2.2.1]heptane-3-carboxylic acid (190 mg, 0.788 mmol) was dissolved in DMF (5.0 mL). DIEA (254 mg, 1.97 mmol) and HATU (270 mg, 1.71 mmol) were added. The reaction mixture was stirred for 15 minutes, and benzyl 4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-3-phenylpropanamido)benzylcarbamate (336 mg, 0.394 mmol) was added. The reaction mixture was stirred for 2 hours at room temperature at which time LCMS analysis indicated the completion of the reaction. The reaction mixture was purified by reverse phase HPLC using C18 column, eluted with 10-90% acetonitrile-H₂O containing 0.05% TFA. The fractions containing the desired product were pooled and lyophilized to obtain (1R,3S,4S)-tert-butyl 3-(((S)-1-(((3R,4S,5S)-1-((S)-2-((1R,2R)-3-(((S)-1-((4- ((((benzyloxy)carbonyl)amino)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate, MS m/z 1080.5 (M+1), Retention time 1.56 minutes.

The Boc protected product (88 mg, 0.081 mmol) was dissolved in methanolic HCl (3 M, 6.0 mL). The solvent was slowly evaporated under reduced pressure. LCMS analysis indicated the completion of the deprotection reaction. Acetonitrile and water were added and the resulting solution was lyophilized to obtain benzyl 4-((S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanamido)benzylcarbamate as a HCl salt, MS m/z 980.5 (M+1).

Step 4: Benzyl 4-((S)-2-((2R,3R)-3-((S)-1-((3R,4S,5S)-4-((S)-2-((1R,3S,4S)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanamido)benzylcarbamate (65.8 mg, 0.067 mmol) was dissolved in MeOH (4 mL). Paraformaldehyde (22.8 mg, 0.76 mmol) and acetic acid (0.023 mL, 0.40 mmol) were added, followed by sodium cyanoborohydride (47.7 mg, 0.76 mmol). The reaction mixture was heated to 50° C. with stirring for 1 hour. LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain benzyl 4-((S)-2-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanamido)benzylcarbamate, MS m/z 994.5 (M+1). Retention time 1.21 minutes.

Step 5: Benzyl 4-((S)-2-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-phenylpropanamido)benzylcarbamate (48 mg, 0.048 mmol) was dissolved in MeOH (5.0 mL), and flashed under N₂. Pd/C (20.5 mg, 10% Pd) was added. The reaction vessel was evacuated and backfilled with H₂. This operation was repeated five times to replace the reaction atmosphere with H₂. The reaction mixture was stirred for 2 hours at room temperature under H₂ LCMS analysis indicated the completion of the reaction. The reaction mixture was filtered, and concentrated to obtained (1R,3S,4S)—N—((S)-1-(((3R,4S,5S)-1-((S)-2-((1R,2R)-3-(((S)-1-((4-(aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide, MS m/z 860.5 (M+1). Retention time 0.86 minutes. The product thus obtained was used in the next step without further purification.

Step 6: (1R,3S,4S)—N—((S)-1-(((3R,4S,5S)-1-((S)-2-((1R,2R)-3-(((S)-1-((4-(Aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.1.1]heptane-3-carboxamide (12 mg, 0.014 mmol) and 2,5-dioxopyrrolidin-1-yl 4-((2,5-dioxo-2,5-dihydro-1H-pyrrol-1-yl)methyl)cyclohexanecarboxylate (5.6 mg, 0.017 mmol) were dissolved in DMF (1 mL), and DIEA (10.8 mg, 0.084 mmol) was added. The reaction mixture was stirred for 1 hour at room temperature. LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain compound 40, MS m/z 1079.5 (M+1). Retention time 1.12 minutes.

### Example 3-79

### (1R,3S,4S)—N—((S)-1-(((3R,4S,5S)-1-((S)-2-((1R,2R)-3-(((S)-1-((4-((3-(2-(2-(2,5-Dioxo-2,5-dihydro-1H-pyrrol-1-yl)ethoxy)ethoxy)propanamido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (41)

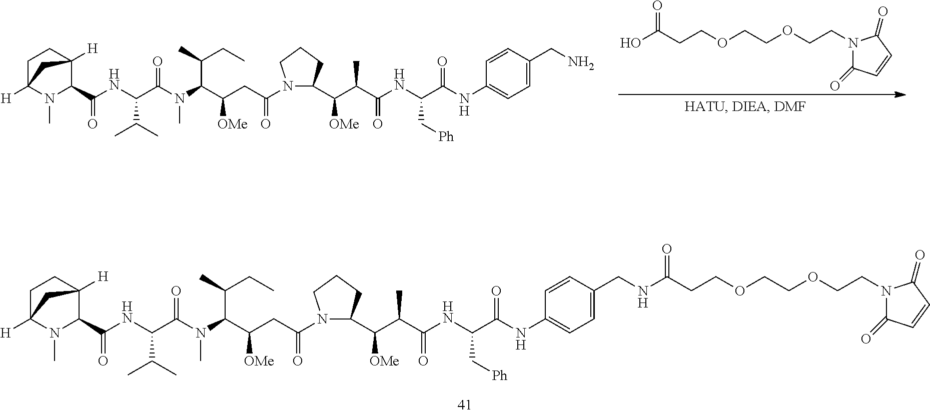 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CN)cc1)OC)N(C)C(=O)[C@@H](NC(=O)[C@@H]1[C@H]2CC[C@H](C2)N1C)C(C)C ; O=C(O)CCOCCOCCN1C(=O)C=CC1=O ; CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CNC(=O)CCOCCOCCN2C(=O)C=CC2=O)cc1)OC)N(C)C(=O)[C@@H](NC(=O)[C@@H]1[C@H]2CC[C@H](C2)N1C)C(C)C]

To 3-(2-(2-(2,5-dioxo-2,5-dihydro-1H-pyrrol-1-yl)ethoxy)ethoxy)propanoic acid (7.2 mg, 0.028 mmol) in DMF (1.5 ml) were added DIEA (10.8 mg, 0.084 mmol) and HATU (8.0 mg, 0.021 mmol). After 10 minutes, (1R,3S,4S)—N—((S)-1-(((3R,4S,5S)-1-((S)-2-((1R,2R)-3-(((S)-1-((4-(aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (12 mg, 0.014 mmol) was added. The reaction mixture was stirred for 2 hours at room temperature. The crude was purified by reverse phase HPLC using C18 column, eluted with 10-90% acetonitrile-H₂O containing 0.05% TFA. The fractions containing the desired product were pooled and lyophilized to obtain compound 41, MS m/z 1099.5 (M+1). Retention time 1.07 minutes.

### Example 3-80

### (1R,3S,4S)—N—((S)-1-(((3R,4S,5S)-1-((S)-2-((1R,2R)-3-(((S)-1-((4-((2,5-Dioxo-2,5-dihydro-1H-pyrrol-1-yl)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (42)

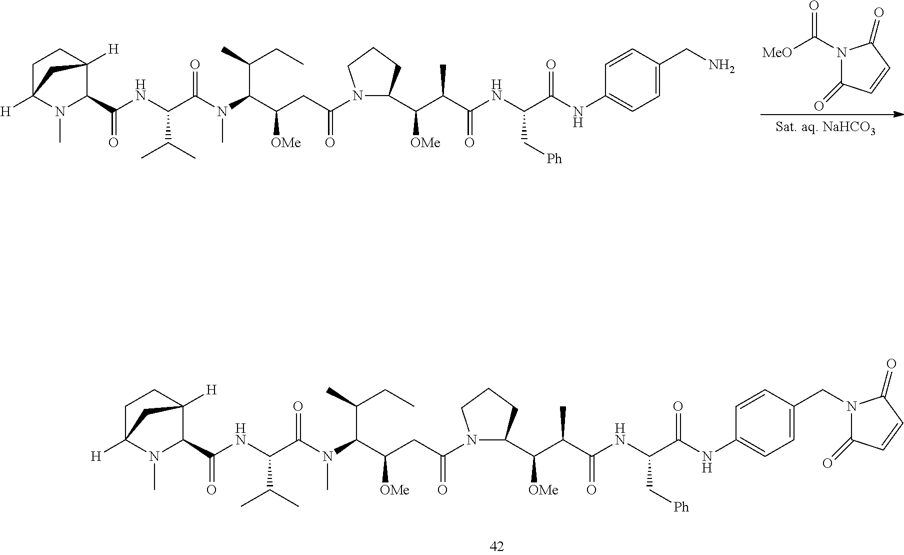 [SMILES: CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CN)cc1)OC)N(C)C(=O)[C@@H](NC(=O)[C@@H]1[C@H]2CC[C@H](C2)N1C)C(C)C ; COC(=O)N1C(=O)C=CC1=O ; CC[C@H](C)[C@@H]([C@@H](CC(=O)N1CCC[C@H]1[C@H](OC)[C@@H](C)C(=O)N[C@@H](Cc1ccccc1)C(=O)Nc1ccc(CN2C(=O)C=CC2=O)cc1)OC)N(C)C(=O)[C@@H](NC(=O)[C@@H]1[C@H]2CC[C@H](C2)N1C)C(C)C]

To saturated aqueous NaHCO₃ (3.0 mL) was added (1R,3S,4S)—N—((S)-1-(((3R,4S,5S)-1-((S)-2-((1R,2R)-3-(((S)-1-((4-(Aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.1.1]heptane-3-carboxamide (20.0 mg, 0.023 mmol). The resulting suspension was cooled to 0° C., and methyl 2,5-dioxo-2,5-dihydro-1H-pyrrole-1-carboxylate (14.4 mg, 0.093 mmol) was added. The reaction mixture was stirred at 0° C. for 1.5 hours. The crude was purified by reverse phase HPLC using C18 column, eluted with 10-90% acetonitrile-H₂O containing 0.05% TFA. The fractions containing the desired product were pooled and lyophilized to obtain compound 42, MS m/z 940.5 (M+1). Retention time 1.13 minutes.

### Example 3-81

### (1R,3S,4S)—N—((S)-1-(((3R,4S,5S)-1-((S)-2-((1R,2R)-3-(((S)-1-((4-((3-(6-(2,5-Dioxo-2,5-dihydro-1H-pyrrol-1-yl)hexyl)ureido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (43)

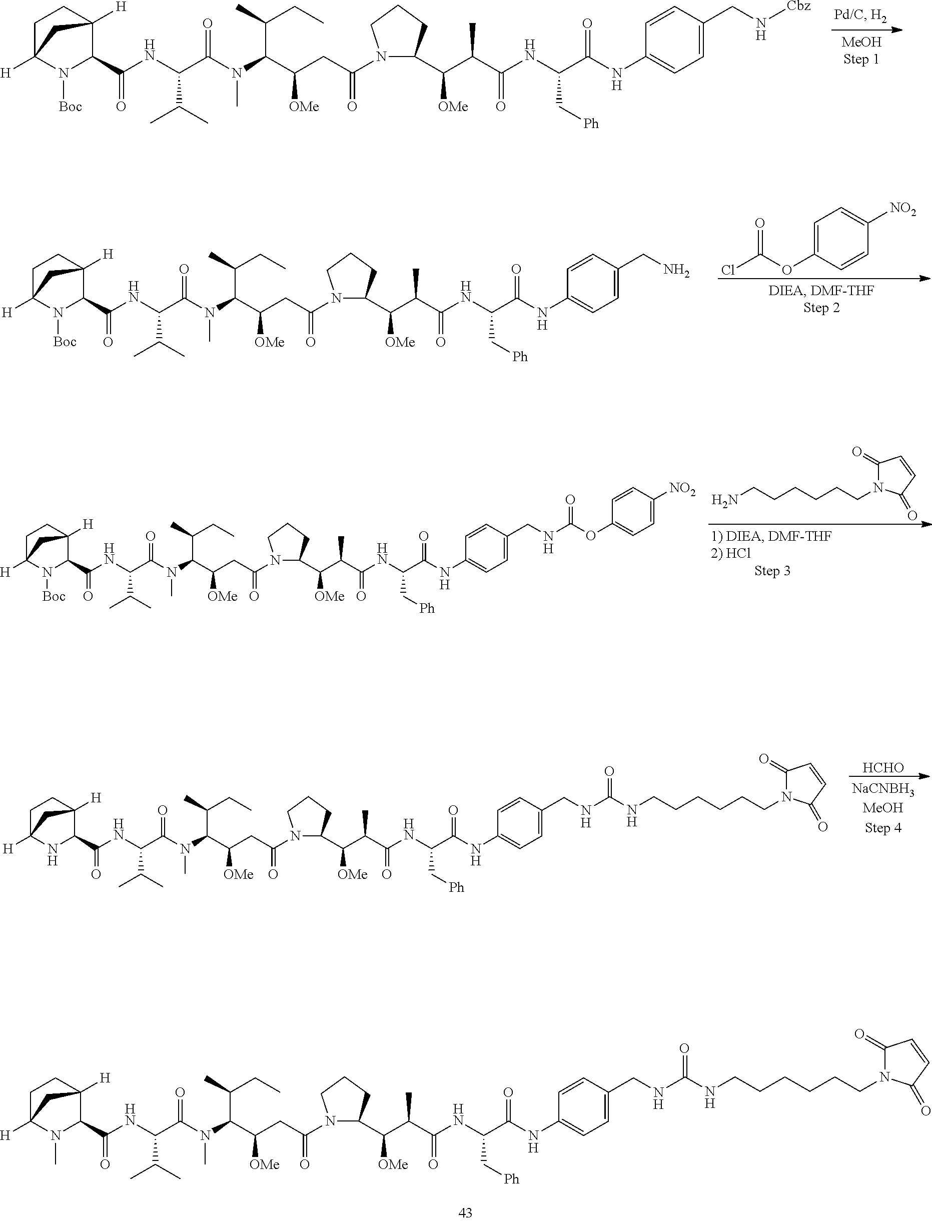

Step 1: (1R,3S,4S)-tert-Butyl 3-(((S)-1-(((3R,4S,5S)-1-((S)-2-((1R,2R)-3-(((S)-1-((4- ((((Benzyloxy)carbonyl)amino)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate (211 mg, 0.195 mmol) was dissolved in MeOH (10 mL). Pd/C (41.6 mg, 10% Pd) was added. The reaction vessel was evacuated and backfilled with H₂. This operation was repeated five times to replace the reaction atmosphere with H₂. The reaction mixture was stirred for 2 hours at room temperature under H₂ LCMS analysis indicated the completion of the reaction. The reaction mixture was filtered and concentrated to afford (1R,3S,4S)-tert-butyl 3-(((S)-1-(((3R,4S,5S)-1-((S)-2-((1R,2R)-3-(((S)-1-((4-(aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate, MS m/z 946.6 (M+1)), which was used in the next step without purification.

Step 2: (1R,3S,4S)-tert-Butyl 3-(((S)-1-(((3R,4S,5S)-1-((S)-2-((1R,2R)-3-(((S)-1-((4-(aminomethyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate (30 mg, 0.032 mmol) was dissolved in DMF (3 ml) and THF (3 ml). Then DIEA (20.5 mg, 0.16 mmol) and 4-nitrophenyl carbonochloridate (12.8 mg, 0.063 mmol) were added. The reaction mixture was stirred for 2 hours at room temperature. LC/MS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain (1R,3S,4S)-tert-butyl 3-(((S)-1-(((3R,4S,5S)-3-methoxy-1-((S)-2-((1R,2R)-1-methoxy-2-methyl-3-(((S)-1-((4-((((4-nitrophenoxy)carbonyl)amino)methyl)phenyl)amino)-1-oxo-3-phenylpropan-2-yl)amino)-3-oxopropyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate, MS m/z 1111.5 (M+1). Retention time 1.54 minutes.

Step 3: To (1R,3S,4S)-tert-Butyl 3-(((S)-1-(((3R,4S,5S)-3-methoxy-1-((S)-2-((1R,2R)-1-methoxy-2-methyl-3-(((S)-1-((4-((((4-nitrophenoxy)carbonyl)amino)methyl)phenyl)amino)-1-oxo-3-phenylpropan-2-yl)amino)-3-oxopropyl)pyrrolidin-1-yl)-5-methyl-1-oxoheptan-4-yl)(methyl)amino)-3-methyl-1-oxobutan-2-yl)carbamoyl)-2-azabicyclo[2.2.1]heptane-2-carboxylate (13.7 mg, 0.012 mmol) dissolved in DMF (1.0 mL) and THF (1.0 mL) were added 1-(6-aminohexyl)-1H-pyrrole-2,5-dione (14.5 mg, 0.074 mmol) and DIEA (31.9 mg, 0.25 mmol). The reaction mixture was stirred for 4 hours at room temperature. Te LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain (1R,3S,4S)-tert-butyl 3-(((S)-1-(((3R,4S,5S)-1-((S)-2-((1R,2R)-3-(((S)-1-((4-((3-(6-(2,5-dioxo-2,5-dihydro-1H-pyrrol-1- yl)hexyl)ureido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)carbamoyl)-2-azabicyclo[2.1.1]heptane-2-carboxylate, MS m/z 1168.6 (M+1). The Boc protected product thus obtained was dissolved in methanolic HCl (3 M, 2.0 mL). The solvent was removed slowly under reduced pressure. LCMS analysis indicated the completion of the reaction. The residue was dissolved in Acetonitrile and water and lyophilized to afford (1R,3S,4S)—N—((S)-1-(((3R,4S,5S)-1-((S)-2-((1R,2R)-3-(((S)-1-((4-((3-(6-(2,5-dioxo-2,5-dihydro-1H-pyrrol-1-yl)hexyl)ureido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide as a HCl salt, MS m/z 1068.6 (M+1). Retention time 1.09 minutes

Step 4: (1R,3S,4S)—N—((S)-1-(((3R,4S,5S)-1-((S)-2-((1R,2R)-3-(((S)-1-((4-((3-(6-(2,5-Dioxo-2,5-dihydro-1H-pyrrol-1-yl)hexyl)ureido)methyl)phenyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.1.1]heptane-3-carboxamide (8.4 mg, 0.0079 mmol) was dissolved in MeOH(1.5 mL). Paraformaldehyde (2.7 mg, 0.089 mmol) and acetic acid (0.0027 mL, 0.046 mmol) were added, followed by sodium cyanoborohydride (5.6 mg, 0.089 mmol). The reaction mixture was heated to 50° C. for 1 hour with stirring. The crude was purified by reverse phase HPLC using C18 column, eluted with 10-90% acetonitrile-H₂O containing 0.05% TFA. The fractions containing the desired product were pooled and lyophilized to obtain compound 43, MS m/z 1082.6 (M+1). Retention time 1.11 minutes.

### Example 3-82

### (1R,3S,4S)—N—((S)-1-(((3R,4S,5S)-1-((S)-2-((1R,2R)-3-(((S)-1-((4-((6-(2,5-Dioxo-2,5-dihydro-1H-pyrrol-1-yl)hexanamido)methyl)phenyl)(methyl)amino)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (44)

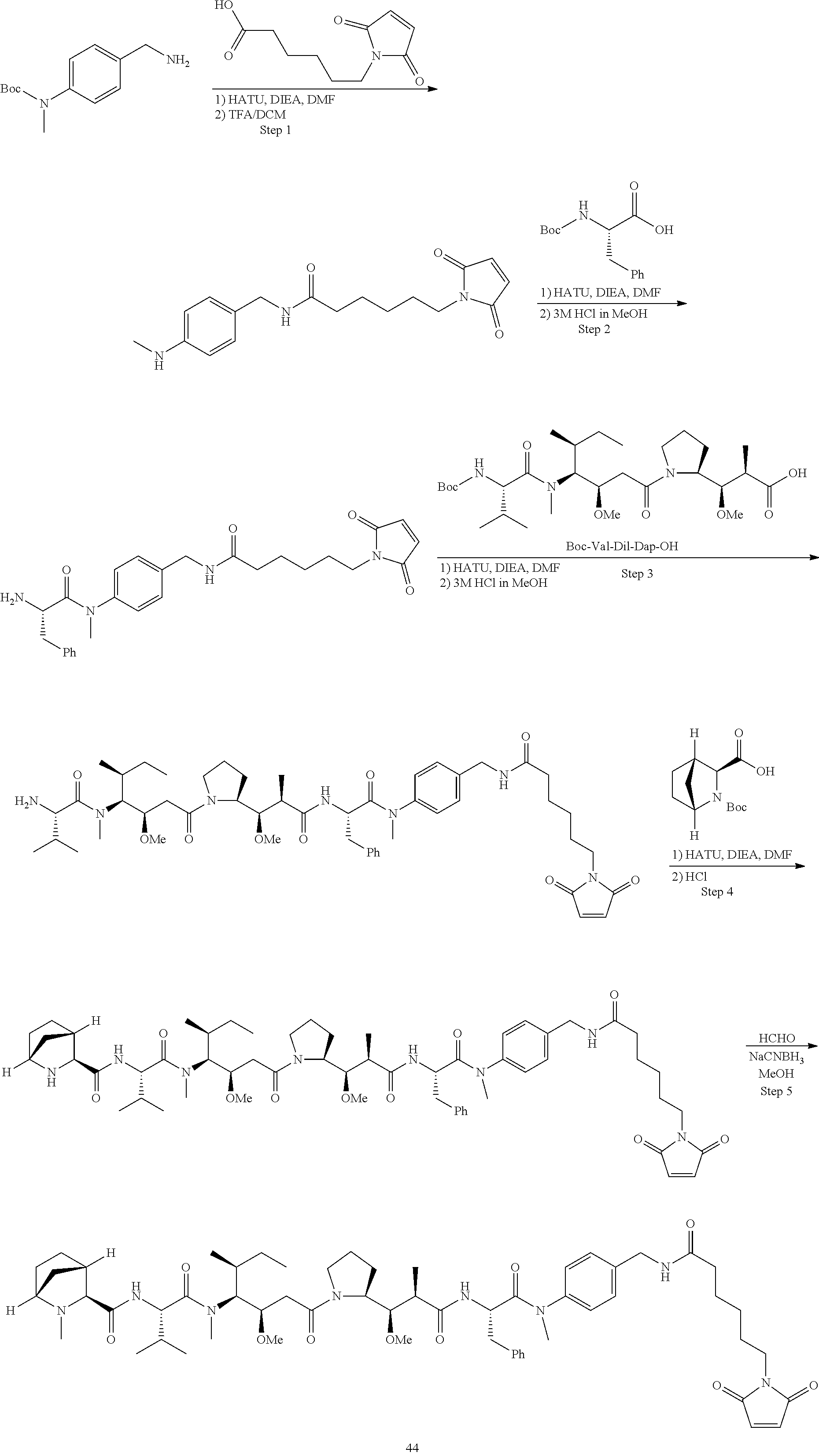

Step 1: 6-(2,5-Dioxo-2,5-dihydro-1H-pyrrol-1-yl)hexanoic acid (349 mg, 1.65 mmol) were dissolved in DMF (10 mL). Then DIEA (820 mg, 6.35 mmol) and HATU (579 mg, 1.52 mmol) were added and the reaction mixture was stirred at room temperature for 10 minutes. tert-Butyl (4-(aminomethyl)phenyl)(methyl)carbamate (300 mg, 1.27 mmol) was then added. The reaction mixture was stirred for 1 hour at room temperature. EtOAc (30 mL) was added to the reaction. Then the reaction mixture was washed with saturated aqueous NaHCO₃. The aqueous layer was extracted with EtOAc (2×30 mL). The combined organic phase was washed with H₂O (5×10 mL), dried with MgSO₄, concentrated and purified by ISCO (EtOAc/Hexane 0-80%). The desired product, MS m/z 374.2 (M+1-tBu), retention time 1.156 minutes, was obtained as a yellow oil. The product was dissolved in DCM (3 mL) and treated with TFA (1 mL). After 1 hour at room temperature, solvents were removed under reduced pressure. The residue was taken up in acetonitrile and H₂O and lyophilized to obtained 6-(2,5-dioxo-2,5-dihydro-1H-pyrrol-1-yl)-N-(4-(methylamino)benzyl)hexanamide as a yellow solid (MS m/z 330.2 (M+1), Retention time 0.61 minutes).

Step 2: To (S)-2-((tert-butoxycarbonyl)amino)-3-phenylpropanoic acid (219 mg, 0.827 mmol) dissolved in DMF (5 mL) were added DIEA (356 mg, 2.76 mmol) and HATU (288 mg, 0.758 mmol). After stirred for 10 minutes at room temperature, 6-(2,5-dioxo-2,5-dihydro-1H-pyrrol-1-yl)-N-(4-(methylamino)benzyl)hexanamide (227 mg, 0.689 mmol) was added. The reaction mixture was stirred for 2 hours at room temperature. EtOAc (20 mL) was added to the reaction. Then the reaction mixture was washed with saturated aqueous NaHCO₃. The aqueous layer was extracted with EtOAc (2×20 mL). The combined organic phase was washed with H₂O (5×10 mL), dreid over anhydrous MgSO₄, concentrated and purified by ISCO (EtOAc/Hexane, 0-75%), affording the desired product. MS m/z 577.3 (M+1). Retention time 1.19 minutes. ¹H NMR (400 MHz, DMSO-d₆): δ 10.00 (s, 1H), 8.24 (t, J=6.0 Hz, 1h), 7.52 (d, j=8.4 Hz, 2H), 7.32-7.09 (m, 7H), 7.01 (s, 2H), 4.31 (m, 1H), 4.19 (d, J=6.0 Hz, 2H), 3.38 (t, J=7.0 Hz, 2H), 3.17 (d, J=7.2 Hz, 2H), 3.00 (m, 1H), 2.85 (m, 1H), 2.10 (t, J=7.4 Hz, 2H), 1.54-1.44 (m, 4H), 1.31 (s, 9H), 1.22-1.15 (m, 4H). The product was dissolved in 3M HCl in MeOH (5 mL). Solvents were removed slowly under reduced pressure. The residue was taken up in acetonitrile and H₂O and lyophilized to obtained (S)—N-(4-(2-amino-N-methyl-3-phenylpropanamido)benzyl)-6-(2,5-dioxo-2,5-dihydro-1H-pyrrol-1-yl)hexanamide as HCl salt. MS m/z 477.2 (M+1). Retention time 0.83 minutes.

Step 3: To Boc-Val-Dil-Dap-OH (347 mg, 0.607 mmol) dissolved in DMF (4 mL) were added DIEA (261 mg, 2.02 mmol) and HATU(282 mg, 0.49 mmol) were. After stirred for 15 minutes at room temperature (S)—N-(4-(2-amino-N-methyl-3-phenylpropanamido)benzyl)-6-(2,5-dioxo-2,5-dihydro-1H-pyrrol-1-yl)hexanamide (193 mg, 0.404 mmol) was added. The reaction mixture was stirred for 2 hours at room temperature. The reaction mixture was purified by reverse-phase HPLC to afford the desired product, N-(4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-N-methyl-3-phenylpropanamido)benzyl)-6-(2,5-dioxo-2,5-dihydro-1H-pyrrol-1-yl)hexanamide. MS m/z 1030.5 (M+1). Retention time 1.430 minutes. The product was dissolved in 3M methanolic HCl (3 mL). Solvents were removed under reduced pressure. The residue was taken up in acetonitrile and H₂O and lyophilized to obtained the desired product N-(4-((S)-2-((2R,3R)-3-((S)-1-((3R,4S,5S)-4-((S)-2-amino-N,3-dimethylbutanamido)-3-methoxy-5-methylheptanoyl)pyrrolidin-2-yl)-3-methoxy-2-methylpropanamido)-N-methyl-3-phenylpropanamido)benzyl)-6-(2,5-dioxo-2,5-dihydro-1H-pyrrol-1-yl)hexanamide as HCl salt. MS m/z 930.5 (M+1). Retention time 1.07 minutes.

Step 4-5: Following the same procedure as described for preparation of compound 30 and compound 31, compound 44 was obtained. MS m/z 1067.6 (M+1). Retention time 1.10 minutes.

### Example 3-83

### (1R,3S,4S)—N—((S)-1-(((3R,4S,5S)-1-((S)-2-((1R,2R)-3-(((S)—N-1-(3-Azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (45)

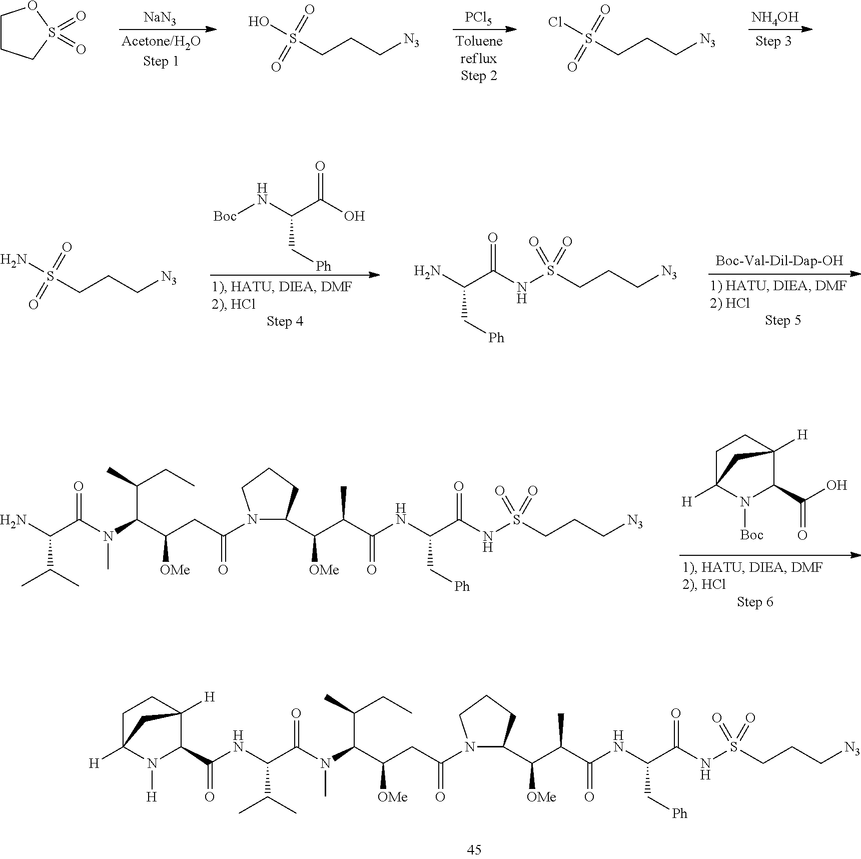

Step 1: To a stirred solution of sodium azide (3.50 g, 53.8 mmol) in water (25 mL) was added a solution of 1,3-propane sulfone (6.10 g, 50.0 mmol) in acetone (25 mL). The reaction mixture was stirred at room temperature for 24 hours and concentrated to dryness. The resulting solid was suspended in diethyl ether (100 mL) and stirred at reflux for 1 hour. The suspension was cooled to room temperature and the solid was collected by filtration, washed with acetone and diethyl ether, and dried under vacuum, affording 3-azido-1-propanesulfonic acid. MS m/z 188.1(M+1). ¹H NMR (400 MHz, CD₃OD): δ 3.47 (t, J=6.8 Hz, 2H), 2.87 (t, J=7.6 Hz, 2H), 2.07-2.00 (m, 2H).

Step 2: 3-Azido-1-propanesulfonic acid (2.07 g, 13.0 mmol) was suspended in toluene. PCl₅ (2.61 g, 13.0 mmol) was added. The mixture was heated at reflux for 3 hours. The reaction mixture was cooled to room temperature, and filtered to remove insolubles. The filter cake was washed with DCM. The combined filtrates were concentrated to give 3-azidopropane-1-sulfonyl chloride as a dark yellow oil, which was used in the next step without further purification.

Step 3: To NH₄OH (5 mIL) cooled at 0° C. was added 3-azidopropane-1-sulfonyl chloride (1.75 g, 9.53 mmol). After 10 minutes, The reaction mixture was warmed to room temperature and stirred at the same temperature for 3 hours. The oily mixture became clear. The reaction mixture was extracted with EtOAc three times. The organic phase was washed with brine, dried over anhydrous MgSO₄, and concentrated. The residual solvent was further removed under high vacuum for 18 hours to give 3-azidopropane-1-sulfonamide. MS m/z 187.1 (M+1). ¹H NMR (400 MHz, CDCl₃): δ 4.83 (s, 2H), 3.51 (t, J=6.4 Hz, 2H), 3.23 (t, J=7.6 Hz, 2H), 2.17-2.10 (m, 2H).

Step 4: (S)-2-((tert-Butoxycarbonyl)amino)-3-phenylpropanoic acid (100 mg, 0.38 mmol) was dissolved in DMF (4 mL), followed by addtion of DIEA (0.395 mL, 2.26 mmol) and HATU (358 mg, 0.940 mmol). After 15 minutes, 3-azidopropane-1-sulfonamide (186 mg, 1.13 mmol) was added. The reaction mixture was stirred for 2 hours at which time LCMS analysis indicated the completion of the reaction. The crude was purified by reverse phase HPLC using C18 column, eluted with 10-90% acetonitrile-H₂O containing 0.05% TFA. The fractions containing the desired roduct were pooled and lyophilized to obtain (S)-tert-butyl (1-(3-azidopropylsulfonamido)-1-oxo-3-phenylpropan-2-yl)carbamate. MS m/z 312.1 (M+1-Boc). Retention time 1.15 minutes. The product thus obtained (72.4 mg. 0.176 mmol) was dissolved in 3M methanolic HCl (5 mL). The solvent was removed under reduced pressure. The residue was taken up in acetonitrile and H₂O and lyophilized to give (S)-2-amino-N-((3-azidopropyl)sulfonyl)-3-phenylpropanamide as a pinkish yellowish solid. MS m/z 312.1 (M+1). ¹H NMR (400 MHz, CD₃OD): δ 7.42-7.31 (m, 5H), 4.16-4.13 (m, 1H), 3.51-3.47 (m, 4H), 3.32-3.26 (m, 1H), 3.13-3.08 (m, 1H), 2.00-1.94 (m, 2H).

Step 5: To Boc-Val-Dil-Dap-OH (195 mg, 0.34 mmol) dissolved in DMF (4 mL) were added DIEA (132 mg, 1.02 mmol) and HATU (108 mg, 0.28 mmol). The reaction mixture was stirred for 15 minutes at room temperature before (S)-2-amino-N-((3-azidopropyl)sulfonyl)-3-phenylpropanamide (59.2 mg, 0.17 mmol) was added. The reaction mixture was stirred for additional 2 hours at room temperature. The crude was puridfied by reverse-phase HPLC to afford the desired product (95 mg, 65% yield, MS m/z 865.4 (M+1), Retention time 1.43 minutes). The product was dissolved in 3M HCl in MeOH (3 mL). Solvents were removed under vacuum. Then acetonitrile and H₂O were added to the residue and the solution was lyophilized to obtained the desired product, (S)-1-(((3R,4S,5S)-1-((S)-2-((1R,2R)-3-(((S)—N-1-(3-azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-2-amino-3-methyl-1-oxobutane. MS m/z 765.4 (M+1). Retention time 1.04 minutes.

Step 6: To (1R,3S,4S)-2-(tert-butoxycarbonyl)-2-azabicyclo[2.2.1]heptane-3-carboxylic acid (16.5 mg, 0.068 mmol) in DMF (2.0 mL) were added DIEA (17.6 mg, 0.137 mmol) and HATU (21.6 mg, 0.057 mmol). The reaction mixture was stirred at room temperature for 10 minutes before (S)-1-(((3R,4S,5S)-1-((S)-2-((1R,2R)-3-(((S)—N-1-(3-azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-2-amino-3-methyl-1-oxobutane (20 mg, TFA salt, 0.023 mmol) was added. The reaction mixture was stirred for 2 hours at room temperatrue at which time LCMS analysis indicated the completion of the reaction. The crude was purified by reverse phase HPLC using C18 column, eluted with 10-90% ACN—H₂O containing 0.05% TFA. The fractions containing the desired product were pooled and lyophilized to obtain (1R,3S,4S)—N—((S)-1-(((3R,4S,5S)-1-((S)-2-((1R,2R)-3-(((S)—N-1-(3-azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-(tert-butoxycarbonyl)-2-azabicyclo[2.2.1]heptane-3-carboxamide. MS m/z 988.5 (M+1). Retention time 1.51 minutes. The product thus obtained (9.4 mg. 0.0095 mmol) was dissolved in methanolic HCl (3M, 2.0 mL). The solvent was removed slowly under reduced pressure. The residue was dissolved in acetonitrile and H₂O and lyophilized to give compound 45 as a HCl salt. MS m/z 888.5 (M+1). Retention time 1.10 minutes.

### Example 3-84

### (1R,3S,4S)—N—((S)-1-(((3R,4S,5S)-1-((S)-2-((1R,2R)-3-(((S)—N-1-(3-Azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (46)

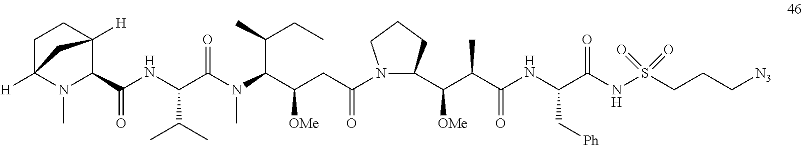 [SMILES: CC[C@H](C)[C@@H]([C@@H](CC(=O)N1CCC[C@H]1[C@H](OC)[C@@H](C)C(=O)N[C@@H](Cc1ccccc1)C(=O)NS(=O)(=O)CCCN=[N+]=[N-])OC)N(C)C(=O)[C@@H](NC(=O)[C@@H]1[C@H]2CC[C@H](C2)N1C)C(C)C]

(1R,3S,4S)—N—((S)-1-(((3R,4S,5S)-1-((S)-2-((1R,2R)-3-(((S)—N-1-(3-Azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-azabicyclo[2.2.1]heptane-3-carboxamide (45) (8.8 mg, 0.0099 mmol) was dissolved in MeOH (2.0 mL). Paraformaldehyde (10.1 mg, 0.337 mmol) and acetic acid (0.0102 mL) were added, followed by sodium cyanoborohydride (21.2 mg, 0.337 mmol). The reaction mixture was heated at 50° C. with stirring for 1 hour. Additional paraformaldehyde (10.1 mg, 0.337 mmol), acetic acid (0.0102 mL) and sodium cyanoborohydride (21.2 mg, 0.337 mmol) were added. After 1 hour at 50° C., LCMS analysis indicated the completion of the reaction. The crude was purified by reverse phase HPLC using C18 column, eluted with 10-90% ACN—H₂O containing 0.05% TFA. The fractions containing the desired product were pooled and lyophilized to obtain compound 46. MS m/z 902.5 (M+1). Retention time 1.12 minutes.

### Example 3-85

### (1R,3S,4S)—N—((S)-1-(((3R,4S,5S)-1-((S)-2-((1R,2R)-3-(((S)—N-1-(3-(4-((2,5-Dioxo-2,5-dihydro-1H-pyrrol-1-yl)methyl)-1H-1,2,3-triazol-1-yl)propylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (47)

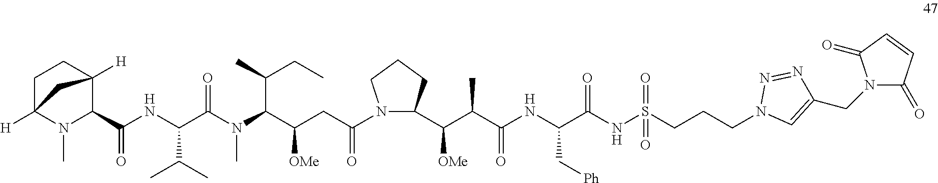 [SMILES: CC[C@H](C)[C@@H]([C@@H](CC(=O)N1CCC[C@H]1[C@H](OC)[C@@H](C)C(=O)N[C@@H](Cc1ccccc1)C(=O)NS(=O)(=O)CCCn1cc(CN2C(=O)C=CC2=O)nn1)OC)N(C)C(=O)[C@@H](NC(=O)[C@@H]1[C@H]2CC[C@H](C2)N1C)C(C)C]

A solution of (1R,3S,4S)—N—((S)-1-(((3R,4S,5S)-1-((S)-2-((1R,2R)-3-(((S)—N-1-(3-azidopropylsulfonamido)-1-oxo-3-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (46) (5.2 mg, 0.0058 mmol), 1-(prop-2-yn-1-yl)-1H-pyrrole-2,5-dione (1.56 mg, 0.012 mmol) and CuSO₄ (0.7 mg, 0.004 mmol) in DMF (2.0 mL) and H₂O (0.5 mL) was treated with L-ascorbic acid sodium salt (2.5 mg, 0.014 mmol) and stirred at room temperature for 2 hours. Additional CuSO4 (0.7 mg, 0.004 mmol) and L-ascorbic acid sodium salt (2.5 mg, 0.014 mmol) were added. After additional 2 hours at room temperature, LCMS analysis indicated the completion of the reaction. The crude was purified by reverse phase HPLC using C18 column, eluted with 10-90% acetonitrile-H₂O containing 0.05% TFA. The fractions containing the desired product were pooled and lyophilized to obtain compound 47. MS m/z 1037.4 (M+1). Retention time 1.00 minutes.

### Example 3-86

### 6-(2,5-Dioxo-2,5-dihydro-1H-pyrrol-1-yl)hexyl hydrogen ((R)-1-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-phenylethyl)phosphonate (48)

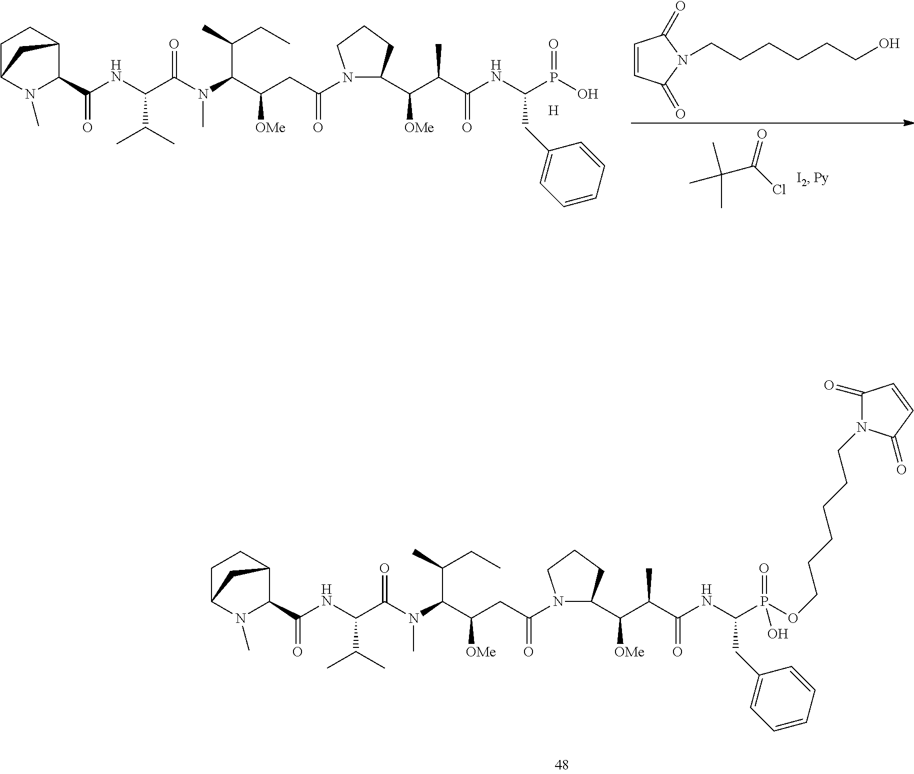

To ((R)-1-((2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-phenylethyl)phosphinic acid 9 (10.2 mg, 0.011 mmol) in pyridine (2 ml) was added 1-(6-hydroxyhexyl)-1H-pyrrole-2,5-dione (13.5 mg, 0.068 mmol) and then pivaloyl chloride (40 mg, 0.332 mmol). The reaction mixture was stirred at room temperature for 0.5 hour and the reaction was monitored by LCMS until 90% of the phosphinic acid disappeared. Then a freshly prepared 1₂ solution in 5% H₂O in pyridine was added. Once the oxidation step was complete, pyridine was removed by high vacuum. The crude was dissolved in acetonitrile and the crude was purified by reverse phase HPLC, C18 column, eluted with 10-60% acetonitrile-H₂O, containing 0.05% TFA. The fractions containing desired product were concentrated to obtain compound 48. MS m/z 971.5 (M+1). Retention time 1.038 minutes.

### Example 3-87

### (1R,3S,4S)—N—((S)-1-(((3R,4S,5S)-1-((S)-2-((1R,2R)-3-(((S)-1-(3-(6-(2,5-Dioxo-2,5-dihydro-1H-pyrrol-1-yl)hexanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.1.1]heptane-3-carboxamide (90)

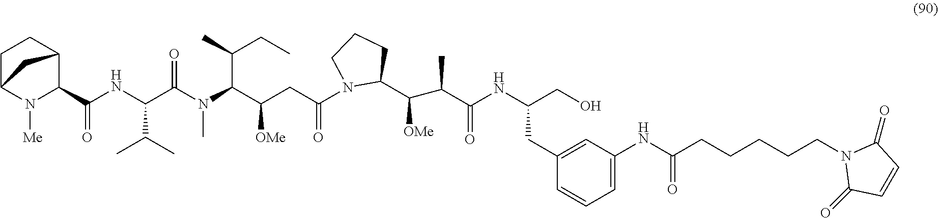 [SMILES: CC[C@H](C)[C@@H]([C@@H](CC(=O)N1CCC[C@H]1[C@H](OC)[C@@H](C)C(=O)N[C@H](CO)Cc1cccc(NC(=O)CCCCCN2C(=O)C=CC2=O)c1)OC)N(C)C(=O)[C@@H](NC(=O)[C@@H]1[C@H]2CC[C@H](C2)N1C)C(C)C]

EMCA (1.3 mg, 0.006 mmol) in DMF (0.5 ml) was treated with DIEA (0.006 ml, 0.03 mmol) and HATU (2.3 mg, 0.006 mmol) at rt for 10 min, and then compound (52) TFA salt (6 mg, 0.006 mmol) in DMF (0.5 ml) was added. The reaction was stirred at rt for 16 h. The crude was purified by preparative HPLC (10-45% acetonitrile-H₂O containing 0.05% TFA) to obtain compound (90) as TFA salt. MS m/z 950.6 (M+H). Retention time 0.934 min.

### Example 3-88

### 4-((S)-2-((S)-2-(6-(2,5-Dioxo-2,5-dihydro-1H-pyrrol-1-yl)hexanamido)-3-methylbutanamido)-5-ureidopentanamido)benzyl (3-((S)-2-((2R,3R)-3-((S)-1-((3R,4S,5S)-4-((S)—N,3-dimethyl-2-((1R,3S,4S)-2-methyl-2-azabicyclo[2.1.1]heptane-3-carboxamido)butanamido)-3-methoxy-5-methylheptanoyl)pyrrolidin-2-yl)-3-methoxy-2-methylpropanamido)-3-hydroxypropyl)phenyl)carbamate (91)

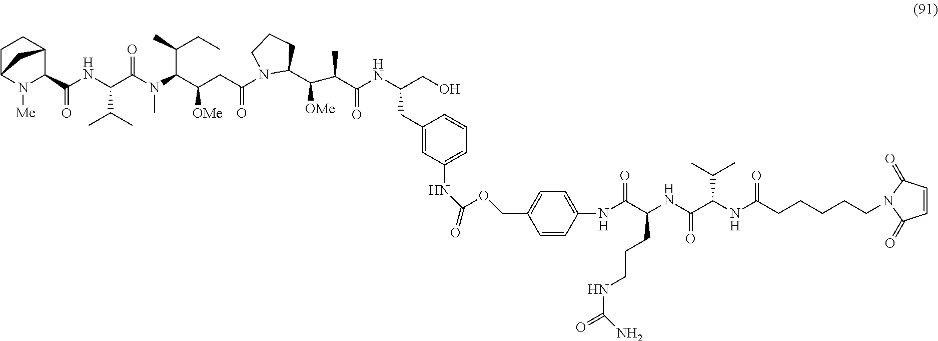 [SMILES: CC[C@H](C)[C@@H]([C@@H](CC(=O)N1CCC[C@H]1[C@H](OC)[C@@H](C)C(=O)N[C@H](CO)Cc1cccc(NC(=O)OCc2ccc(NC(=O)[C@H](CCCNC(N)=O)NC(=O)[C@@H](NC(=O)CCCCCN3C(=O)C=CC3=O)C(C)C)cc2)c1)OC)N(C)C(=O)[C@@H](NC(=O)[C@@H]1[C@H]2CC[C@H](C2)N1C)C(C)C]

Pyridine (0.25 ml) was added to (52) TFA salt (6 mg, 0.006 mmol), MC-Val-Cit-PAB-PNP (13 mg, 0.018 mmol) in DMF (1 ml), followed by HOAT (0.8 mg, 0.006 mmol) and DIEA (13 mg, 0.098 mmol).The reaction was stirred at 40° C. for 48 h. The crude was purified by preparative HPLC (25-40% acetonitrile-H₂O containing 0.05% TFA) to obtain compound (91) as TFA salt. MS m/z 678.6 (M/2+H). Retention time 0.959 min.

### Example 3-89

### (1R,3S,4S)—N—((S)-1-(((3R,4S,5S)-3-Methoxy-1-((S)-2-((1R,2R)-1-methoxy-2-methyl-3-oxo-3-(((S)-1-phenyl-3-(N-(6-(2,5-dioxo-2,5-dihydro-1H-pyrrol-1-yl)hexanoyl)-sulfamoylpropan)-2-yl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (92)

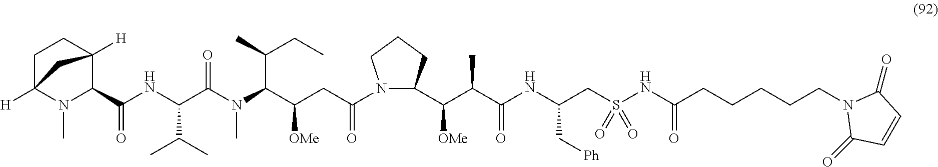 [SMILES: CC[C@H](C)[C@@H]([C@@H](CC(=O)N1CCC[C@H]1[C@H](OC)[C@@H](C)C(=O)N[C@@H](Cc1ccccc1)CS(=O)(=O)NC(=O)CCCCCN1C(=O)C=CC1=O)OC)N(C)C(=O)[C@@H](NC(=O)[C@@H]1[C@H]2CC[C@H](C2)N1C)C(C)C]

EMCA (12.1 mg, 0.057 mmol) was dissolved in DMF (1 ml). DIEA (0.0024 ml) and HATU (19.7 mg, 0.052 mmol) were added. Then (1R,3S,4S)—N—((S)-1-(((3R,4S,5S)-3-methoxy-1-((S)-2-((1R,2R)-1-methoxy-2-methyl-3-oxo-3-(((S)-1-phenyl-3-sulfamoylpropan-2-yl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide ((55), 26.4 mg, 0.029 mmol) in DMF (2 ml) was added. The reaction was stirred at rt for 2 h. Then additional EMCA (12.1 mg, 0.057 mmol), DIEA (0.0024 ml) and HATU (19.7 mg, 0.052 mmol) were added. After 2h, EMCA (12.1 mg, 0.057 mmol), DIEA (0.0024 ml) and HATU (19.7 mg, 0.052 mmol) were added again. Then the reaction was heated at 50° C. for 2 h. The reaction was cooled down, and additional EMCA (12.1 mg, 0.057 mmol), DIEA (0.0024 ml) and HATU (19.7 mg, 0.052 mmol) were added. The reaction was stirred for 16 h at rt. LCMS indicated approximately 20% of (55) was converted to the product. The reaction mixture was purified by preparative HPLC (30-50% acetonitrile-H₂O containing 0.05% TFA) to obtain (1R,3S,4S)—N—((S)-1-(((3R,4S,5S)-3-methoxy-1-((S)-2-((1R,2R)-1-methoxy-2-methyl-3-oxo-3-(((S)-1-phenyl-3-(N-(6-(2,5-dioxo-2,5-dihydro-1H-pyrrol-1-yl)hexanoyl)-sulfamoylpropan)-2-yl)amino)propyl)pyrrolidin-1-yl)-5-methyl-1-oxoheptan-4-yl)(methyl)amino)-3-methyl-1-oxobutan-2-yl)-2-methyl-2-azabicyclo[2.2.1]heptane-3-carboxamide (92). MS m/z 998.5 (M+1). Retention time 1.041 min.

### Example 3-90

### (1R,4S)—N—((S)-1-(((3R,4S,5S)-1-((S)-2-((3R,4R,7S)-7-Benzyl-20-(4-((2,5-dioxo-2,5-dihydro-1H-pyrrol-1-yl)methyl)-1H-1,2,3-triazol-1-yl)-4-methyl-5,8-dioxo-2,12,15,18-tetraoxa-6,9-diazaicosan-3-yl)pyrrolidin-1-yl)-3-methoxy-5-methyl-1-oxoheptan-4-yl)(methyl)amino)-3-methyl-1-oxobutan-2-yl)-2-methyl-2-azabicyclo[2.1.1]heptane-3-carboxamide (93)

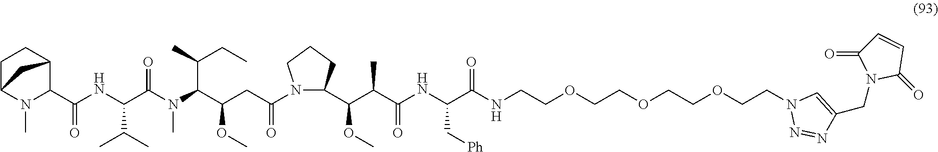 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCOCCOCCOCCn1cc(CN2C(=O)C=CC2=O)nn1)OC)N(C)C(=O)[C@@H](NC(=O)C1[C@H]2CC[C@H](C2)N1C)C(C)C]

Step 1: (S)-2-((t-Butoxycarbonyl)amino)-3-phenylpropanoic acid (175 mg, 0.66 mmol) in DMF (4 ml) was treated with DIEA (0.48 ml, 2.75 mmol) and HATU (230 mg, 0.605 mmol) for 15 min, followed by addition of 2-(2-(2-(2-azidoethoxy)ethoxy)ethoxy)ethanamine (120 mg, 0.55 mmol). The reaction was stirred overnight. The crude was purified by preparative HPLC (20-70% acetonitrile-H₂O containing 0.05% TFA) to obtain (S)-t-butyl (1-azido-13-oxo-15-phenyl-3,6,9-trioxa-12-azapentadecan-14-yl)carbamate. MS m/z 466.3 (M+1). Retention time 1.170 min.

Step 2: (S)-t-Butyl (1-azido-13-oxo-15-phenyl-3,6,9-trioxa-12-azapentadecan-14-yl)carbamate (117 mg, 0.251 mmol) was dissolved in methanolic HCl (3M, 5 ml). The solvent was slowly removed by evaporation, resulting in complete removal of the Boc group. The residual solvent was further removed under reduced pressure overnight to obtain (S)-2-amino-N-(2-(2-(2-(2-azidoethoxy)ethoxy)ethoxy)ethyl)-3-phenylpropanamide as HCl salt. MS m/z 366.1 (M+1). Retention time 0.858 min.

Step 3: (2R,3R)-3-((S)-1-((3R,4S,5S)-4-((S)—N,3-Dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid (Step 2, Example 3-12, 8 mg, 0.01 mmol) was in DMF (1 ml) was treated with DIEA (0.011 ml, 0.066 mmol) and HATU (4.63 mg, 0.012 mmol) for 15 min, followed by addition of (S)-2-amino-N-(2-(2-(2-(2-azidoethoxy)ethoxy)ethoxy)ethyl)-3-phenylpropanamide (5.3 mg, 0.013 mmol) in DMF (1 ml). The reaction was stirred for 2 h at rt. The crude was purified by preparative HPLC (20-70% acetonitrile-H₂O containing 0.05% TFA) to obtain (1R,4S)—N—((S)-1-(((3R,4S,5S)-1-((S)-2-((3R,4R,7S)-20-azido-7-benzyl-4-methyl-5,8-dioxo-2,12,15,18-tetraoxa-6,9-diazaicosan-3-yl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide. MS m/z 956.5 (M+1). Retention time 1.051 min.

Step 4: To the product obtained in step 3 (6.2 mg, 0.0058 mmol) and 1-(prop-2-yn-1-yl)-1H-pyrrole-2,5-dione (1.6 mg, 0.012 mmol) in t-BuOH (1 ml) and water (1 ml) were added sodium L-ascorbate (1.1 mg, 0.0058 mmol) in 0.2 ml H₂O and CuSO₄ (0.2 mg, 0.001 mmol) in 0.1 ml water were added. The reaction mixture was stirred at rt for 4 h, and then purified by preparative HPLC (20-70% acetonitrile-H₂O containing 0.05% TFA) to obtain compound (93). MS m/z 1091.6 (M+1). Retention time 0.980 min.

### Example 3-91

### (1R,4S)—N—((S)-1-(((3R,4S,5S)-1-((S)-2-((3R,4R,7S)-7-Benzyl-17-(2,5-dioxo-2,5-dihydro-1H-pyrrol-1-yl)-4-methyl-5,8-dioxo-2,12,15-trioxa-6,9-diazaheptadecan-3-yl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (94)

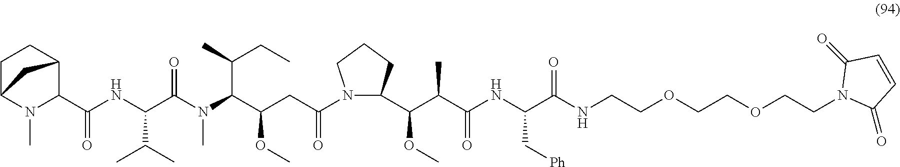 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCOCCOCCN1C(=O)C=CC1=O)OC)N(C)C(=O)[C@@H](NC(=O)C1[C@H]2CC[C@H](C2)N1C)C(C)C]

Step 1: t-Butyl (2-(2-(2-aminoethoxy)ethoxy)ethyl)carbamate(250 mg, 1.0 mmol) and methyl 2,5-dioxo-2,5-dihydro-1H-pyrrole-1-carboxylate (156 mg, 1.0 mmol) were combined in saturated aqueous NaHCO₃ (10 ml) and stirred for 1.5 h at 0° C. The reaction mixture was acidified to pH 2 with hydrochloric acid (2 M) and extracted with EtOAc. The organic phase was washed with brine, dried with MgSO₄, and concentrated. The crude was purified by ISCO using 0-4% MeOH/DCM to give t-butyl (2-(2-(2-(2,5-dioxo-2,5-dihydro-1H-pyrrol-1-yl)ethoxy)ethoxy)ethyl)carbamate as a colorless oil. MS m/z 229.2 (M+1-Boc). Retention time 0.963 min. ¹H NMR (400 MHz, Chloroform-d) δ 6.71 (s, 2H), 5.04 (bs, 1H), 3.74 (t, J=5.4 Hz, 2H), 3.64 (t, J=5.4 Hz, 2H), 3.61-3.59 (m, 2H), 3.56-3.54 (m, 2H), 3.50 (t, J=5.2 Hz, 2H), 3.31-3.26(m, 2H), 1.44 (s, 9H).

Step 2: t-Butyl (2-(2-(2-(2,5-dioxo-2,5-dihydro-1H-pyrrol-1-yl)ethoxy)ethoxy)ethyl)carbamate (184 mg, 0.56 mmol) in DCM (2 ml) was treated with TFA (0.4 ml) at 0° C. for 30 min and then at rt for 2h. The reaction mixture was concentrated to give 1-(2-(2-(2-aminoethoxy)ethoxy)ethyl)-1H-pyrrole-2,5-as TFA salt. MS m/z 229.2(M+1). Retention time 0.353 min.

Step 3: Boc-L-Phe-OH (30 mg, 0.113 mmol) in DMF (1 ml) was activated with DIEA (88 mg) and HATU (43 mg, 0.113 mmol) for 15 min, and 1-(2-(2-(2-aminoethoxy)ethoxy)ethyl)-1H-pyrrole-2,5-dione TFA salt (46.4 mg) in DMF (1 ml) was added. The reaction mixture was stirred at rt for 2 h and then purified by preparative HPLC (20-70% acetonitrile-H₂O containing 0.05% TFA) to obtain (S)-t-butyl (1-((2-(2-(2-(2,5-dioxo-2,5-dihydro-1H-pyrrol-1-yl)ethoxy)ethoxy)ethyl)amino)-1-oxo-3-phenylpropan-2-yl)carbamate. MS m/z 476.2(M+1). Retention time 1.091 min. This product (31 mg, 0.065 mmol) in DCM (2 ml) was treated with TFA (0.2 ml) at 0° C. for 30 min and then at rt for 2 h. The reaction mixture was concentrated to give (S)-2-amino-N-(2-(2-(2-(2,5-dioxo-2,5-dihydro-1H-pyrrol-1-yl)ethoxy)ethoxy)ethyl)-3-phenylpropanamide as TFA salt. MS m/z 376.2(M+1). Retention time 0.649 min.

Step 4: To (2R,3R)-3-((S)-1-((3R,4S,5S)-4-((S)—N,3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid TFA salt (Step 2, Example 3-12) (7.2 mg, 0.010 mmol) in DMF (1 ml) were added DIEA (7.7 mg) and HATU (4.18 mg, 0.011 mmol). The reaction was stirred for 15 min, and (S)-2-amino-N-(2-(2-(2-(2,5-dioxo-2,5-dihydro-1H-pyrrol-1-yl)ethoxy)ethoxy)ethyl)-3-phenylpropanamide TFA salt (6.3 mg, 0.013 mmol) in DMF (1 ml) was added. The reaction mixture was stirred at rt for 2 h and purified by preparative HPLC (20-70% acetonitrile-H₂O containing 0.05% TFA) to obtain compound (93) as TFA salt. MS m/z 966.5 (M+1). Retention time 1.016 min.

### Example 3-92

### (1R,3S,4S)—N—((S)-1-(((3R,4S,5S)-1-((S)-2-((3R,4R,7S)-7-Benzyl-14-(2,5-dioxo-2,5-dihydro-1H-pyrrol-1-yl)-4-methyl-5,8-dioxo-2,12-dioxa-6,9-diazatetradecan-3-yl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (95)

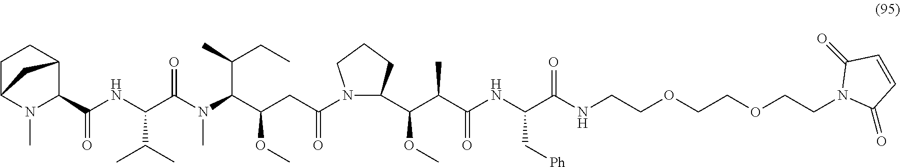 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCOCCOCCN1C(=O)C=CC1=O)OC)N(C)C(=O)[C@@H](NC(=O)[C@@H]1[C@H]2CC[C@H](C2)N1C)C(C)C]

Compound (95) was prepared by the method described for compound (94) using t-butyl (2-(2-aminoethoxy)ethyl)carbamate in place of t-butyl (2-(2-(2-aminoethoxy)ethoxy)ethyl)carbamate. MS m/z 922.5 (M+1). Retention time 1.044 min.

### Example 3-93

### Synthesis of Comparative Cytotoxic Peptide MC-MMAF

Synthesis of (S)-2-((2R,3R)-3-((S)-1-((3R,4S,5S)-4-((S)-2-((S)-2-(6-(2,5-dioxo-2,5-dihydro-1H-pyrrol-1-yl)-N-methylhexanamido)-3-methylbutanamido)-N,3-dimethylbutanamido)-3-methoxy-5-methylheptanoyl)pyrrolidin-2-yl)-3-methoxy-2-methylpropanamido)-3-phenylpropanoic acid (MC-MMAF)

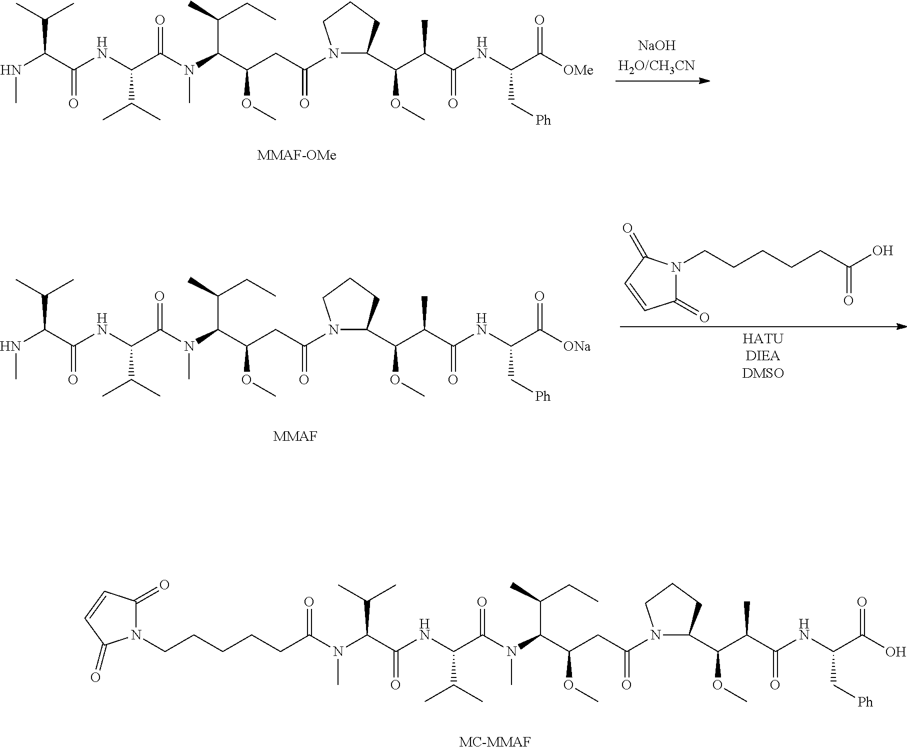

MMAF-OMe (135 mg, Concortis Biosystems) was dissoved in CH3CN (10 mL). To the resulting clear solution was added 5 mL water, followed by 0.375 mL of 1N aqueous sodium hydroxide (certified, Fisher Scientific). The reaction mixture was stirred magnetically at 21° C. for 18 hours, at which time LCMS analysis indicated a complete reaction. The reaction mixture mixture was frozen and lyophilized, affording MMAF sodium salt. LCMS retention time 0.911 minutes. MS (ESI+) m/z 732.5 (M+1). The whole MMAF sodium salt thus obtained in previous reaction was dissoved in 10 mL DMSO. In a separate reaction vessel, 6-(2,5-dioxo-2,5-dihydro-1H-pyrrol-1-yl)hexanoic acid (95 mg) was treated with HATU (165 mg) and DIEA (0.126 mL) in 3.0 mL DMSO at at 21° C. for 25 min. The whole reaction mixture of the activated ester was added to the solution of MMAF sodium salt, and The reaction mixture was stirred at the same temperature for 3 hours. The reaction mixture mixture was partitioned between 40 mL of EtOAc and 20 mL of 5% aqueous citric acid. The organic layer was separated, and the aqueous layer was extracted with 20 mL of EtOAc. The combined organic layers were washed with 10 mL saturated aqueous NaCl, dryed over anhydrous MgSO4, filtered and concentrated under reduced pressure. The residue was purified on an ISCO CombiFlash instrument using an ISCO C18gold 15.5 g column. The desired material was eluted with 50% CH₃CN in H₂O. The fractions containing the desired product was combined and lyophilized, affording compound as white solid. LCMS retention time 1.392 minutes. MS (ESI+) m/z 925.6 (M+1).

### Example 4

### Generation and Characterization of P-Cadherin Antibody Drug Conjugates

### Example 4-1

### Synthesis of Ketone Functionalized Antibody NOV169N31Q-KB (Ab2)

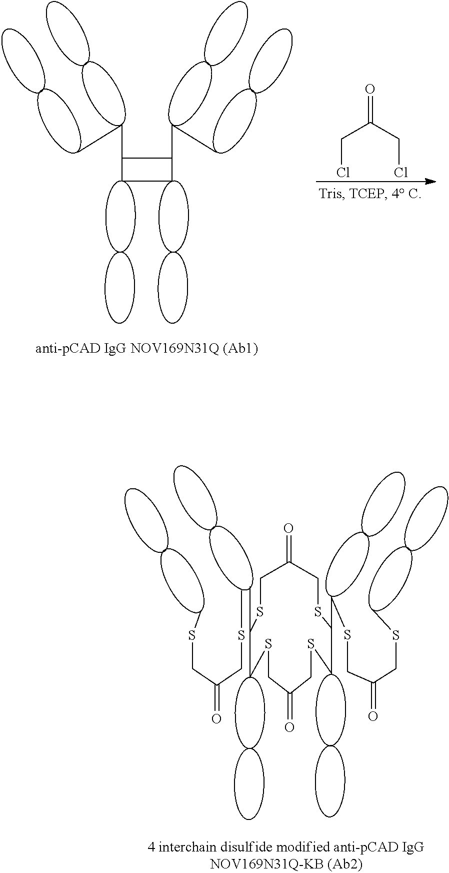

Tris(2-carboxyethyl)phosphine hydrochloride (0.393 mg, 1.37 μmol) was added slowly to a precooled solution of anti-P-cadherin IgG (Ab1, 17.0 mg, 0.114 μmol, 100 μL), and 1,3-dichloroacetone (1.83 mg, 0.014 mmol) in Tris buffer (1200 μl) at to 4° C. The resulting mixture was kept at 4° C. for 4 h. The mixture was concentrated via Amicon membrane filteration (10K) with PBS (pH 7.4) as the eluting buffer. The resulting sample was subsequently desalted using a Zeba spin column 7K MWCO with PBS (pH7.4) as the eluting buffer to give the title compound Ab2 (15.2 mg, 89%). LCMS (Analytical Method A; Eluent A: water+0.1% Formic acid, Eluent B: Acetonitrile+0.1% Formic acid, Gradient: from 2 to 98% B in 3 min. Column: Proswift Monolith 4.6*50 mm 50° C.); 146019 (after deglycosylation by PNGase F (New England biolab)).

Analytical Method

### Method A:

Eluent A: water+0.1% Formic acid, Eluent B: Acetonitrile+0.1% Formic acid.
Gradient: from 2 to 98% B in 3 min. Column: Proswift Monolith 4.6*50 mm 50° C.

### Example 4-2

### Synthesis of ADC NOV169N31Q-KB-22

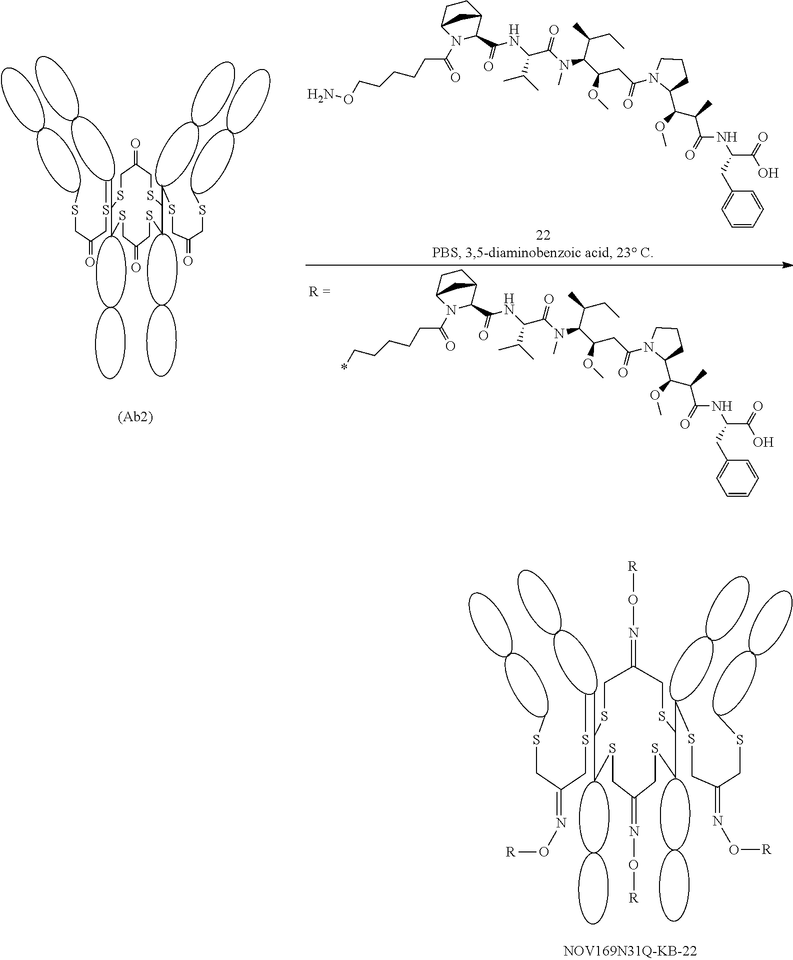

(S)-2-((2R,3R)-3-((S)-1-((3R,4S,5S)-4-((S)-2-((1R,3S,4S)-2-(6-(Aminooxy)hexanoyl)-2-azabicyclo[2.2.1]heptane-3-carboxamido)-N,3-dimethylbutanamido)-3-methoxy-5-methylheptanoyl)pyrrolidin-2-yl)-3-methoxy-2-methylpropanamido)-3-phenylpropanoic acid (22) (7, 3730 μg, 3.79 μmol, 24.9 μL in DMSO) and 3,5-diaminobenzoic acid in DMSO (6490 μg, 42 μmol, 16.1 μL) were added to a solution of Ab2 in PBS pH 7.4 (22.6 mg, 0.152 μmol) at room temperature. The resulting mixture was kept at 23° C. for 15 h. The mixture was was subsequently desalted using a Zeba spin column 7K MWCO with PBS (pH7.4) as the eluting buffer for three times, and filtered through 10K Amicon membrane to give the conjugate NOV169N31Q-KB-22 (17.7 mg, 80% based on protein recovery). The average DAR was determined as 3.77 based on LCMS. LCMS (method A); 152480 (DAR4) (intact mass).

### Example 4-3

### Preparation of Antibody Drug Conjugates Using Anti-P-Cadherin-Antibodies NOV169N31Q-152/375C and NEG0012-152/375C

Numerous methods for conjugating linker-payloads to antigen binding moieties are known in the art (reviewed in for example: Antibody-Drug Conjugate, Methods in Molecular Biology, Vol. 1045, Editor L. Ducry, Humana Press (2013)). In this example, two payload compounds (compound 10, compound 77) were conjugated to two engineered cysteine residues (heavy chain mutations E152C and S375C numbered according to the EU system) in the HC constant regions of anti-P-cadherin antibodies (NOV169N31Q-152/375C and NEG0012-152/375C). The two anti-P-cadherin-152/375C constructs were expressed in HEK293 cells in a transient fashion. In another example, the two anti-P-cadherin-152/375C constructs were expressed in stably transfected CHO cell lines. Both NOV169N31Q-152/375C and NEG0012-152/S375C were purified by Protein A affinity columns from culture media and polished by preparative size exclusion chromatography. Because engineered Cys in antibodies expressed in mammalian cells are modified by adducts (disulfides) such as glutathione (GSH) and/or cysteine during their biosynthesis (Chen et al. mAb 6:563-571, 2009), the modified Cys in the antibodies as initially expressed is unreactive to thiol reactive reagents such as maleimido or bromo-or iodo-acetamide groups. To conjugate payload drugs to the engineered cysteines in the antibodies, the glutathione or cysteine adducts need to be removed by reducing these disulfides, which generally entails reducing also the native disulfides in the expressed antibody. This can be accomplished by first exposing the antibody to a reducing agent such as dithiothreitol (DTT), TCEP or reduced cysteine followed by a procedure that allows for the re-oxidation of all native disulfide bonds of the antibody to restore and/or stabilize the functional antibody structure. Accordingly, in order to reduce the disulfide bound between the cysteine or GSH adducts of the engineered cysteine residues, freshly prepared DTT was added to purified NOV169N31Q-152/375C and NEG0012-152/375C antibodies to a final concentration of 10 mM. After incubation with DTT at RT for 1 h, the reduced antibodies were loaded to a Sephadex G-25 desalting column equilibrated with PBS to remove DTT. Antibody fractions devoid of DTT were collected and reoxidation incubation was performed at RT for 24 h to allow the reduced antibody to be reoxidized. A reverse phase HPLC was used to monitor the reoxidation process. Antibodies were loaded onto a PLRP-S column (4000 Å, 50 mm×2.1 mm, Agilent) heated to 80° C. and elution of antibodies from the column was carried out by a linear gradient of 30-45% acetonitrile in water containing 0.1% TFA at a flow rate of 1.5 mL/min and was monitored at 280 nm, 254 nm and 215 nm. Another method to remove the reducing reagents is to dialyze the reduced antibodies against PBS and the reduced antibodies can be reoxidized readily during dialysis process. Alternatively, glutathione or cysteine adducts in antibodies can be removed with a large excess of fully reduced cysteine at 20 mM concentration while the antibodies are bound to protein A Sepharose resin. After incubation of the antibody bound resin slurry for 30-60 minutes at room temperature, excess cysteine is then rapidly removed by washing the resin with 50 bed volumes of PBS. The reoxidation of the reduced antibodies are carried out in the presence of CuCl₂ on Protein A resin slurry. All three methods produce similar results.

After re-oxidation, NOV169N31Q-152/375C and NEG0012-152/375C were conjugated with two cytotoxic peptides, compound 10 and compound 77. The payload compounds at 8-10 molar equivalents relative to the antibody were added to re-oxidized NOV169N31Q-152/375C and NEG0012-152/375C in PBS buffer (pH 7.2). After 1 hour incubation at RT, the antibody drug conjugates (ADC) were purified by Protein A affinity column chromatography to remove free payloads. The payload drugs conjugated to NOV169N31Q-152/375C and NEG0012-152/375C were determined by a reverse-phase HPLC as described above and by LC/MS. Average loading of a cytotoxic peptide to an antibody is generally referred to as drug to antibody ratio (DAR). The DAR for a given conjugate represents the average number of drug (payload) molecules attached to a typical antibody containing two light chains and two heavy chains. The DAR value is extrapolated from reverse phase HPLC measurements or from LC-MS analysis. For most linker-payload molecules, ADCs with different number of payload drug molecules attached can readily be resolved by a reverse phase column in HPLC. LC/MS also allows quantitation of the average number of molecules of payload (drug) attached to an antibody in an ADC. For LC-MS analysis, ADCs are typically reduced and deglycosylated. LC separates heavy chain (HC) and light chain (LC) of the reduced antibody according to the number of linker-payload groups per chain. Mass spectral data enables identification of the component species in the mixture, e.g., LC, LC+1, LC+2, HC, HC+1, HC+2, etc. From the average loading on the LC and HC chains, the average DAR can be calculated for an ADC. The resulting DAR values from both reverse phase HPLC and LC/MS are generally in good agreement. The DAR values for various NOV169N31Q-152/375C-10, NOV169N31Q-152/375C-77, NEG0012-152/375C-10, and NEG0012-152/375C-77 ADC preparations range from 3.6 to 4.0. Since there are four engineered cysteines in NOV169N31Q-152/375C and NEG0012-152/375C antibodies DAR values at 3.6 to 4 would indicate that more than 90% of engineered cysteine residues in the two anti-P-cadherin-152/375C antibodies were conjugated with payload drugs. The anti-P-cadherin-152/375C ADC preparations were further analyzed by an analytic size exclusion column (Agilent Bio SEC-3, 300 Å, 7.8×150 mm) to determine their aggregation status. ADC samples prepared from conjugation of compound 10 and compound 77 to NOV169N31Q-152/375C and NEG0012-152/375C contain less than 1% aggregates.

### Example 5

### Affinity of P-Cadherin Antibodies to P-Cadherin

The affinity of various antibodies to P-cadherin and its species orthologues was determined using SPR technology using a Biacore® T100 instrument (GE Healthcare, Pittsburgh, Pa.) or a Biacore® 2000 instrument (GE Healthcare, Pittsburgh, Pa.) using CMS sensor chips.

Briefly, HBS-P⁺ (0.01 M HEPES, pH 7.4, 0.15 M NaCl, 0.05% Surfactant P20) supplemented with 2% Odyssey® blocking buffer (Li-Cor Biosciences, Lincoln, Nebr.) was used as the running buffer for all the experiments on the Biacore® T100 instrument. HBS-P (0.01 M HEPES, pH 7.4, 0.15 M NaCl, 0.005% Surfactant P20) supplemented with 2% Odyssey® blocking buffer was used as the running buffer for all the experiments on the Biacore® 2000 instrument. The immobilization level and analyte interactions were measured by response unit (RU). Pilot experiments were performed to test and confirm the feasibility of the immobilization of Protein A (GE Healthcare, Pittsburgh, Pa.) and the capture of the test antibodies.

For kinetic measurements, experiments were performed in which the antibodies were captured to the sensor chip surface via the immobilized Protein A and the ability of the P-cadherin proteins to bind in free solution was determined. Briefly, 28 μg/ml of Protein A at pH 4.5 was immobilized on a CM5 sensor chip through amine coupling at flow rate of 10 μl/minute on two flow cells to reach 2300-3300 RUs. 0.01-0.25 μg/ml of test antibodies was then injected at 5 μl/min for 3 minute. Captured levels of the antibodies were generally kept below 400 RUs. Subsequently, 6.25-100 nM of P-cadherin ECD was diluted in a 2-fold series and injected at a flow rate of 40 μl/min for 2-4 min over both reference and test flow cells. Table 7 of tested ECDs is listed below. Dissociation of the binding was followed for 5 min. After each injection cycle, the chip surface was regenerated with PBS/6 M Guanidine HCl, pH 7.4 at 100 μl/min for 45 seconds. All experiments were performed at 25° C. and the response data were globally fitted with a simple 1:1 interaction model using Biacore T100 Evaluation Software version 2.0.3 (GE Healthcare) to obtain estimates of on rate (kₐ), off-rate (k_{d}) and affinity (K_{D}) on the Biacore® T100 instrument. Experiments that were ran on the Biacore® 2000 instrument were globally fitted with a simple 1:1 interaction model using Scrubber 2® software version 2.0b (BioLogic Software) to obtain estimates of on rate (kₐ), off-rate (k_{d}) and affinity (K_{D})

TABLE 7
P-cadherin ECD isotype and
source used in Affinity Assay

| ECD | | |
|---|---|---|
| Isotype | Tag | Source |
| Human | C-terminal 6× His | NVS |
| Cyno_1 | C-terminal 6× His | NVS |
| Cyno_2 | C-terminal 6× His | NVS |
| Mouse | C-terminal 6× His | NVS |
| Rat | C-terminal 6× His | NVS |

Table 8 lists the domain binding and affinity of various P-cadherin antibodies disclosed in Table 2. As shown in Table 8, the antibodies NOV169N31Q, NEG0012, NEG0013, NEG0016, NEG0064, and NEG0067 all reacted with the human P-cadherin at the nanomolar level, and have similar affinities for those tested against cynomolgus monkey P-cadherin ECD. All the antibodies cross reacted with the rat except NOV169N31Q.

TABLE 8
Affinity estimates of anti-P-cadherin antibodies obtained
against human P-cadherin and species orthologues

| | | Affinity estimate (K_{D}) | | | |
|---|---|---|---|---|---|
| | | Human | Cyno_1 | Cyno_2 | Rat |
| | | P-cad | P-cad | P-cad | P-cad |
| Antibody ID | | (nM) | (nM) | (nM) | (nM) |
| NOV169N31Q | Naked | 33.3 | 36.8 | 44.5 | No Binding |
| NEG0012 | Naked | 44.8 | 30.2 | 23 | 44.5 |
| NEG0013 | Naked | 37.3 | 21.1 | 17.2 | 37 |
| NEG0016 | Naked | 60 | 26.9 | 33.6 | 60.1 |
| NEG0064 | Naked | 3.66 | 3.46 | 2.9 | 8.99 |
| NEG0067 | Naked | 3.21 | 3.27 | 3.53 | 10.3 |

### Example 6

### NOV169N31Q Selectivity in Biochemical Assays

To examine the potential for off-target cross-reactivity, NOV169N31Q was evaluated for binding to two closely related classical cadherin family members with the highest degree of amino acid sequence identity in their corresponding ECDs: E-cadherin (CDH1) or N-cadherin (CDH2).

To assess specificity of binding to P-cadherin vs E-cadherin and N-cadherin, Maxisorp 384-well plates were o/n at 4° C. with recombinant human E-cadherin or N-cadherin ECD and Fab fragments were assayed using an enzyme-linked immunosorbent assay (ELISA) format. After washing, plates were blocked for 2 h with 5% skim milk in 1× PBST. Fab-containing E. coli lysates were added and binding allowed for 1 h at room temperature (RT). To detect bound Fab fragments, plates were washed 5× with TBST and AP-anti human IgG F(ab′)2 was added in a 1/2500 dilution. After 1 h at RT, plates were washed 5× with TBST and AttoPhos substrate was added according to the manufacturers specifications. Plates were read in an ELISA reader 5 minutes after adding the substrate.

Utilizing ELISA methodology, no significant binding to human E-cadherin or N-cadherin was detected for the anti-P-cadherin antibody candidate NOV169N31Q.

### Example 7

### Assessment of NOV169N31Q Impact on P-Cadherin Function

A study was performed to assess the ability of anti-P-cadherin antibody NOV169N31Q to exert a functional effect on P-cadherin in a cellular assay. P-cadherin is a homotypic cell adhesion molecule expressed on the cell surface of cancer cells, thus a spheroid integrity assay using P-cadherin positive HCT116 cells and P-cadherin negative HT-29 cells was employed to assess potential antagonistic properties of the antibody. The read-out of this assay is shape and tightness of the spheroid, as determined by brightfield microscopy and 7-AAD fluorescence detection of cellular DNA.

HCT116 cells (P-cadherin positive) or HT29 cells (P-cadherin negative) were seeded at a density of 6,000 cells per well in 96-well round bottom ultra-low attachment plates (Corning Cat. #7007), with or without human IgG1 isotype control Ab (10 μg/mL) or NOV169N31Q (10 μg/mL). Cells were placed on an orbital shaker (60 rpm) at 37° C., 5% CO2. 7-Aminoactinomycin D (7-AAD; BD Pharmingen, Cat. #559925) was added 116 hours after cells were plated to label cellular DNA. 7-AAD imaging was performed on a GE IN Cell Analyzer 2000 using the Texas Red filter 132 hours after cells were plated. 7-9 “z” image stacks were taken of each well and the image stacks were collapsed using the IN Cell Developer Toolbox 1.8 program.

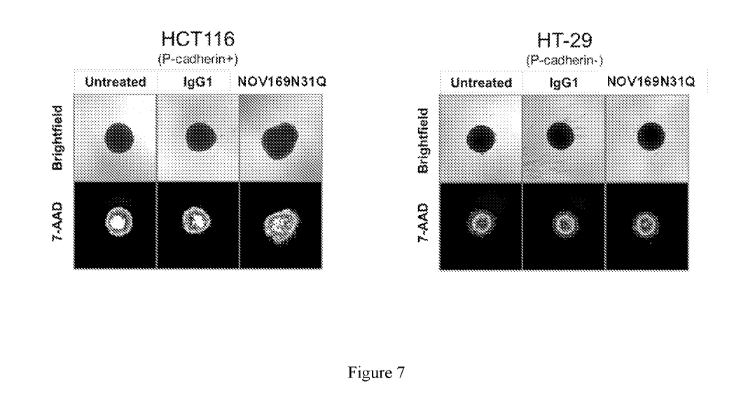

In FIG. 7, NOV169N31Q showed a small, but discernable effect on P-cadherin-mediated cellular adhesion in P-cadherin expressing HCT116 cells, but not in P-cadherin negative HT29 cells, as evidenced by bright field microscopy and spheroid density analysis determined by 7-AAD (DNA-based) imaging. In contrast, a non-specific control human IgG1 antibody had no impact on the integrity of multicellular spheroids. Thus, NOV169N31Q may be a partial antagonist of P-cadherin function in vitro and/or in vivo.

### Example 8

### NOV169N31Q-KB-22 Inhibition of Cell Proliferation and Survival

The ability of NOV169N31Q-KB-22 to inhibit cell proliferation and survival was assessed using the CellTiterGlo® proliferation assay.

The cell lines were cultured in media that is optimal for their growth at 5% CO2, 37° C. in a tissue culture incubator. Prior to seeding for the proliferation assay, the cells were split at least 2 days before the assay to ensure optimal growth density. On the day of seeding, cells were lifted off tissue culture flasks using 0.05% trypsin. Cell viability and cell density were determined using a cell counter (Vi-Cell XR Cell Viability Analyzer, Beckman Coulter). Cells with higher than 85% viability were seeded in white-walled clear bottom 384-well plates (Corning cat #3707) at a density of 500-1,500 cells per well in 40 μL of standard growth media. Wells bordering the edge of plates were filled with media alone in order to minimize the effects of evaporation on well volumes. Plates were incubated at 37° C. overnight in a tissue culture incubator. The next day, free auristatin (me-MMAF), NOV169N31Q-KB-22, and the non-targeting ADC control (IgG1-KB-22) were prepared at 5× in standard growth media. The prepared drug treatments were then added to the cells resulting in final concentrations ranging from 0-50M and a final volume of 50 μL per well. Each drug concentration was tested in quadruplicate. Plates were incubated at 37° C. overnight or for 5 days in a tissue culture incubator, after which cell viability was assessed through the addition of 25 μL of CellTiter Glo® (Promega, cat #G7573), a reagent which lyses cells and measures total adenosine triphosphate (ATP) content. The plates were incubated in the dark at room temperature on an orbital shaker at a speed that provides adequate mixing for 3 minutes to induce cell lysis. Plates were incubated at room temperature for 30-60 minutes to stabilize luminescent signals prior to reading using a luminescence counter (EnVision, Perkin Elmer). To evaluate the effect of the drug treatments, luminescent counts from wells containing untreated cells (100% viability) were used to normalize treated samples. IC50 values were calculated using Graph Pad Prism 6 software. Each cell line was evaluated at least 3 times and representative IC50 values are shown.

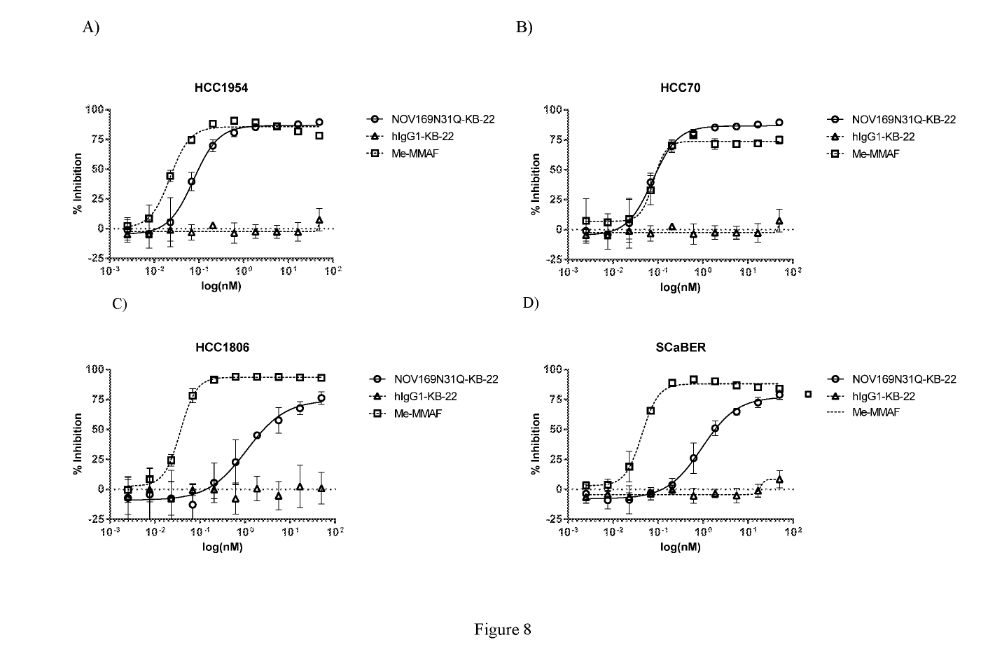

NOV169N31Q-KB-22 has a target average of 4.0 molecules of compound 22 bound to each antibody (Drug to Antibody Ratio, or DAR, of 4.0). The dose-response curves of 4 representative cell lines are shown (FIG. 8). The concentrations of treatment required to inhibit 50% of cell growth or survival (IC50) were calculated, with representative IC50 values of the cell lines tested summarized in Table 9. The unconjugated antibody NOV169N31Q was demonstrated to be neither cytotoxic nor anti-proliferative, while NOV169N31Q-KB-22 potently inhibited proliferation and survival in P-cadherin-expressing cell lines. Neither molecule was active in the P-cadherin negative cell line HT29. In contrast, NOV169N31Q-KB-22 potently inhibited growth of three breast cancer cell lines HCC1954, HCC70, and HCC1806 and one bladder cancer cell line SCaBER. Table 9 summarizes the activity of NOV169N31Q-KB-22 in a panel of cell lines. Compared with the isotype matched non-targeting control ADC (IgG1-KB-22), NOV169N31Q-KB-22 often showed cytotoxic activities toward cell lines that express more than >50,000 cell surface copies of P-cadherin per cell. These studies indicate that the cytotoxic effect of NOV169N31Q-KB-22 is due to the internalized drug moiety component of the ADC and NOV169N31Q-KB-22 specifically targets cells overexpressing P-cadherin.

TABLE 9
NOV169N31Q-KB-22 activity in a panel of
human cancer cell lines (IC₅₀ (nM) values).

| | | Antigen | |
|---|---|---|---|
| | | Density | IC50 (nM) |
| Cell Line | Origin | (×1000) | NOV169N31Q-KB-22 |
| HCC1954 | Breast | 84 | 0.07592 |
| HCC70 | Breast | 66 | 0.3991 |
| HCC1806 | Breast | 68 | 1.094 |
| SCaBER | Bladder | 86 | 0.9456 |

### Example 9

### In Vivo Efficacy of NOV169N31Q-KB-22 Payload Against the HCC70 Triple Negative Breast Cancer (TNBC) Model in Mice

The HCC70 cell line with approximately 66,000 receptors on the surface of each cell, was shown to be sensitive to NOV16931Q-KB-22 in vitro. To demonstrate targeted anti-tumor activity in this model in vivo, the antibodies NOV169N31Q and the hIgG1 isotype control (3207) were conjugated via ketone-bridge to auristatin compound 22. A single IV dose of 0.625 mg/kg and 2.5 mg/kg was administered to female SCID mice harboring established HCC70 tumors. HCC70 tumor xenografts were generated in female SCID mice by subcutaneous injection of 10×10⁶ cells into the right flank of each mouse. When tumors reached 230 mm3, mice were randomized according to tumor volume into treatment groups (n=7). All test agents were administered at the dose levels and schedules indicated, and doses were adjusted to individual mouse body weights. The IV dose volume was 10 ml/kg.

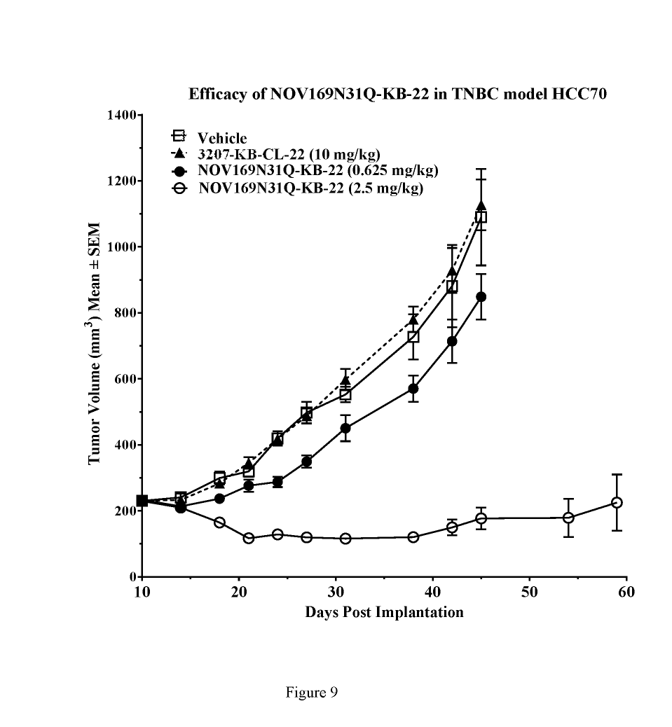

FIG. 9 depicts the results of this in vivo experiment. Single IV dose administration of NOV169N31Q-KB-22 at 0.625 mg/kg resulted in no significant effect on tumor volume at day 47 (One way ANOVA, p>0.5). Moderate tumor growth inhibition was observed in the groups dosed with the NOV169N31Q conjugates containing the compound 22 payload up to approximately 3 weeks post-first dose, however, this effect was shortlived and tumor growth kinetics mirrored the controls on subsequent measurements. At 35 days post first-dose, NOV169N31Q-KB-22 dosed at 2.5 mg/kg showed significant anti-tumor activity, with % regression values of −6.1% . The 2.5 mg/kg groups tested statistically different from the vehicle and isotype controls (P<0.001, ANOVA, day 47). NOV169N31Q-KB-22 dosed at 2.5 mg/kg continued to show a durable response out to 66 days post-treatment, when the study was terminated.

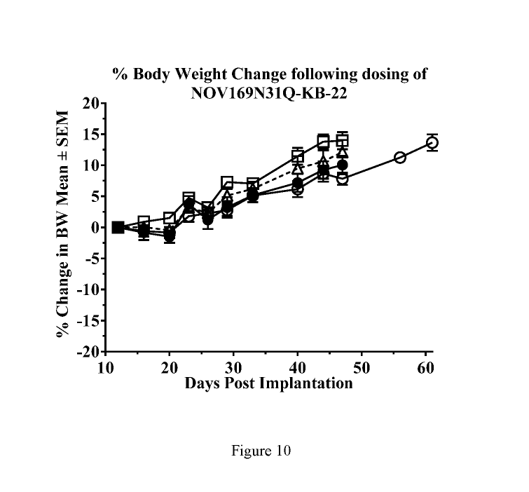

All test agents were tolerated on study and no overt clinical symptoms of toxicities were observed in any of the treatment groups, as expected for an ADC that does not bind mouse P-cadherin. In all groups, body weight gain was observed compared to the mean body weights at randomization, and was similar to that of vehicle-treated mice, as expected for an ADC that does not bind mouse P-cadherin (FIG. 10).

TABLE 10
In vivo efficacy of NOV169N31Q-KB-22 against the HCC70 triple negative breast cancer
(TNBC) model in mice.

| | | | Tumor Response | | | | |
|---|---|---|---|---|---|---|---|
| | | | | | ΔTumor | Host Response | |
| | | | ΔT/ΔC | Regression | volume | Δbody | Survival |
| Test agent | Dose | Schedule | (%) | (%) | (mm³) | weight (%) | (survivors/total) |
| Untreated | NA | N/A | 100 | — | 1090 ± 146 | 14.0 ± 1.1 | 7/7 |
| hIgG1-KB-22 | 2.5 | Single | 105.7 | — | 1127 ± 77 | 12.1 ± 1.9 | 7/7 |
| | | dose/IV | | | | | |
| NOV169N31Q- | 0.625 | Single | 71.8 | — | 849 ± 67 | 10.0 ± 1.6 | 7/7 |
| KB-22 | | dose/IV | | | | | |
| NOV169N31Q- | 2.5 | Single | — | 6.1 | 177 ± 33 | 7.8 ± 1.0 | 7/7 |
| KB-22 | | dose/IV | | | | | |

The effect of the treatment on tumor volumes and body weights are presented as means ± SEM.
The experiment was evaluated on treatment day 35 and day 37 respectively.
*p < 0.001 versus vehicle (Kruskal-Wallis One Way ANOVA on Ranks/Tukey's Test).

### Example 10

### In Vivo Efficacy of NOV169N31Q-152/375C-77 Payload Against the HCC70 Triple Negative Breast Cancer (TNBC) Model in Mice

To assess the novel linker-payload, Compound 77, in the context of a P-cadherin targeting antibody, ADC induced efficacy in the HCC70 xenograft model was assessed in a dose-dependent manner. To demonstrate targeted anti-tumor activity in this model by a potent AuriX linker payload, the antibody NOV169N31Q was conjugated, via site-directed mutageneis of cysteines located within the IgG heavy chain (E152C/S375C), to the following AuriX payload: Compound 77. The full ADC nomenclature including the antibody and linker payload is NOV169N31Q-1521375C-77. A single IV dose of 1 mg/kg and 2 mg/kg was administered to female SCID mice harboring established HCC70 tumors. HCC70 tumor xenografts were generated in female SCID mice by subcutaneous injection of 10×10⁶ cells into the right flank of each mouse. When tumors reached ˜200 mm³, mice were randomized according to tumor volume into treatment groups (n=6). All test agents were administered at the dose levels and schedules indicated (Table 11), and doses were adjusted to individual mouse body weights. The IV dose volume was 10 ml/kg.

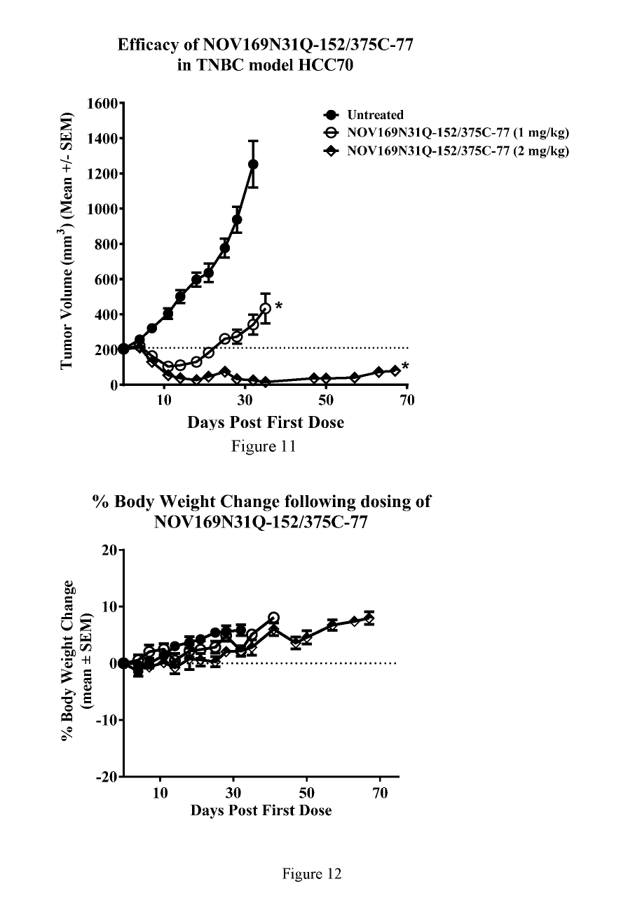

Single IV dose administration of NOV169N31Q-152/375C-77 at 1 and 2 mg/kg resulted in a significant effect on tumor volume at day 32 (One way ANOVA, p<0.005) (FIG. 11). Tumor growth inhibition of 13% AT/AC (% change in treated tumor volume vs. untreated control) was observed in the group dosed at 1 mg/kg and 86% tumor regression was observed in the group dosed at 2 mg/kg. The initial tumor response at 1 mg/kg resulted in tumor regression that was durable for approximately 10 days, with tumor regrowth occurring at day 11 post dose. NOV169N31Q-152/375C-77 when dosed at 2 mg/kg resulted in durable regression that lasted for approximately 70 days, at which point the study was concluded. Both dosed groups tested statistically different from the untreated control group (P<0.005, ANOVA, day 32).

All test agents were tolerated on study and no overt clinical symptoms of toxicities were observed in any of the treatment groups, as expected for an ADC that does not bind mouse P-cadherin. In all groups, body weight gain was observed compared to the mean body weights at randomization, and was similar to that of untreated mice, as expected for an ADC that does not bind mouse P-cadherin (FIG. 12).

TABLE 11
In vivo efficacy of NOV169N31Q-152/375C-77 against the HCC70 triple negative breast
cancer (TNBC) model in mice

| | | | Tumor Response | | | | |
|---|---|---|---|---|---|---|---|
| | | | | | ΔTumor | Host Response | |
| | | | ΔT/ΔC | Regression | volume | ΔBody weight | Survival |
| Test agent | Dose | Schedule | (%) | (%) | (mm3 ± SEM) | (% ± SEM) | (Survivors/total) |
| Untreated | NA | NA | 100 | | 1047.55 ± 132.16 | 5.87 ± 0.97 | 6/6 |
| NOV169N31Q- | 2 | Single | | −86.64 | −176.57 ± 14.92 | 2.15 ± 0.93 | 6/6 |
| 152/375C-77 | | dose/ | | | | | |
| | | IV | | | | | |
| NOV169N31Q- | 1 | Single | 13.09 | | 137.09 ± 49.49 | 2.24 ± 0.64 | 6/6 |
| 152/375C-77 | | dose/ | | | | | |
| | | IV | | | | | |

The effect of the treatment on tumor volumes and body weights are presented as means ± SEM.
The experiment was evaluated on treatment day 32.
*p < 0.001 versus vehicle (Kruskal-Wallis One Way ANOVA on Ranks/Tukey's Test).

### Example 11

### NOV169N31Q-152/375C-77 Inhibition of Cell Proliferation and Survival

The ability of NOV169N31Q-152/375C-77 to inhibit cell proliferation and survival was assessed using the CellTiterGlo® proliferation assay.

The cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cells were lifted off tissue culture flasks using 0.05% trypsin. Cell viability and cell density were determined using a cell counter (Vi-Cell XR Cell Viability Analyzer, Beckman Coulter). Cells with higher than 85% viability were seeded in white-walled clear bottom 384-well plates (Corning cat #3707) at a density of 500-1,500 cells per well in 40 μL of standard growth media. Wells bordering the edge of plates were filled with media alone in order to minimize the effects of evaporation on well volumes. Plates were incubated at 37° C. overnight in a tissue culture incubator. The next day, free auristatin (me-MMAF), NOV169N31Q-152/375C-77, and the non-targeting ADC control (IgG1-1521375C-77) were prepared at 5× in standard growth media. The prepared drug treatments were then added to the cells resulting in final concentrations ranging from 0-50M and a final volume of 50 μL per well. Each drug concentration was tested in quadruplicate. Plates were incubated at 37° C. overnight or for 5 days in a tissue culture incubator, after which cell viability was assessed through the addition of 25 μL of CellTiterGlo® (Promega, cat #G7573), a reagent which lyses cells and measures total adenosine triphosphate (ATP) content. The plates were incubated in the dark at room temperature on an orbital shaker at a speed that provides adequate mixing for 3 minutes to induce cell lysis. Plates were incubated at room temperature for 30-60 minutes to stabilize luminescent signals prior to reading using a luminescence counter (EnVision, Perkin Elmer). To evaluate the effect of the drug treatments, luminescent counts from wells containing untreated cells (100% viability) were used to normalize treated samples. IC50 values were calculated using Graph Pad Prism 6 software. Each cell line was evaluated at least 3 times and representative IC50 values are shown.

NOV169N31Q-152/375C-77 has a target average of 4.0 molecules of compound 77 bound to each antibody (Drug to Antibody Ratio, or DAR, of 4.0). The concentrations of treatment required to inhibit 50% of cell growth or survival (IC50) were calculated, with representative IC50 values of the cell lines tested summarized in Table 12. The unconjugated antibody NOV169N31Q-152/375C was demonstrated to be neither cytotoxic nor anti-proliferative, while NOV169N31Q-152/375C-77 potently inhibited proliferation and survival in P-cadherin-expressing cell lines. Neither molecule was active in the P-cadherin negative cell line HT29. In contrast, NOV169N31Q-152/375C-77 potently inhibited growth of three breast cancer cell lines HCC1954, HCC70, and HCC1806 and one bladder cancer cell line SCaBER. Table 12 summarizes the activity of NOV169N31Q-152/375C-77 in a panel of cell lines. Compared with the isotype matched non-targeting control ADC (IgG1-152/375C-77), NOV169N31Q-152/375C-77 often showed cytotoxic activities toward cell lines that express more than >50,000 cell surface copies of P-cadherin per cell. These studies indicate that the cytotoxic effect of NOV169N31Q-152/375C-77 is due to the internalized drug moiety component of the ADC and NOV169N31Q-152/375C-77 specifically targets cells overexpressing P-cadherin.

TABLE 12
NOV169N31Q-152/375C-77 in a panel of
human cancer cell lines (IC₅₀ (nM) values).

| | | Antigen | IC50 (nM) |
|---|---|---|---|
| | | Density | NOV169N31Q- |
| Cell Line | Origin | (×1000) | 152/375C-77 |
| HCC1954 | Breast | 84 | 0.8964 |
| HCC70 | Breast | 66 | 1.193 |
| HCC1806 | Breast | 68 | 9.532 |
| Scaber | Bladder | 86 | 4.075 |

It is understood that the examples and embodiments described herein are for illustrative purposes only and that various modifications or changes in light thereof will be suggested to persons skilled in the art and are to be included within the spirit and purview of this application and the scope of the appended claims.

1. An antibody that binds human P-cadherin selected from any one of the following:
a. An antibody or antigen binding fragment thereof comprising a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at positions 152 and 375, wherein said cysteine positions are numbered according to the EU system;
b. An antibody or antigen binding fragment thereof comprising a heavy chain variable region that comprises a VH CDR1 of SEQ ID NO: 21, a VH CDR2 of SEQ ID NO: 22, and a VH CDR3 of SEQ ID NO: 23, wherein the CDR is defined in accordance with the Kabat definition; and a light chain variable region that comprises a VL CDR1 of SEQ ID NO: 31, a VL CDR2 of SEQ ID NO: 32, and a VL CDR3 of SEQ ID NO: 33, wherein the CDR is defined in accordance with the Kabat definition, and a modified heavy chain constant region comprising cysteine at positions 152 and 375, wherein said cysteine positions are numbered according to the EU system;
c. An antibody or antigen binding fragment thereof comprising a heavy chain variable region that comprises a VH CDR1 of SEQ ID NO:41, a VH CDR2 of SEQ ID NO:42, and a VH CDR3 of SEQ ID NO:43, wherein the CDR is defined in accordance with the Kabat definition; and a light chain variable region that comprises a VL CDR1 of SEQ ID NO:51, a VL CDR2 of SEQ ID NO:52, and a VL CDR3 of SEQ ID NO:53, wherein the CDR is defined in accordance with the Kabat definition, and a modified heavy chain constant region comprising cysteine at positions 152 and 375, wherein said cysteine positions are numbered according to the EU system;
d. An antibody or antigen binding fragment thereof comprising a heavy chain variable region that comprises a VH CDR1 of SEQ ID NO:61, a VH CDR2 of SEQ ID NO:62, and a VH CDR3 of SEQ ID NO:63, wherein the CDR is defined in accordance with the Kabat definition; and a light chain variable region that comprises a VL CDR1 of SEQ ID NO:71, a VL CDR2 of SEQ ID NO:72, and a VL CDR3 of SEQ ID NO:73, wherein the CDR is defined in accordance with the Kabat definition, and a modified heavy chain constant region comprising cysteine at positions 152 and 375, wherein said cysteine positions are numbered according to the EU system;
e. An antibody or antigen binding fragment thereof comprising a heavy chain variable region that comprises a VH CDR1 of SEQ ID NO:81, a VH CDR2 of SEQ ID NO:82, and a VH CDR3 of SEQ ID NO:83, wherein the CDR is defined in accordance with the Kabat definition; and a light chain variable region that comprises a VL CDR1 of SEQ ID NO:91, a VL CDR2 of SEQ ID NO:92, and a VL CDR3 of SEQ ID NO:93, wherein the CDR is defined in accordance with the Kabat definition, and a modified heavy chain constant region comprising cysteine at positions 152 and 375, wherein said cysteine positions are numbered according to the EU system;
f. An antibody or antigen binding fragment thereof comprising a heavy chain variable region that comprises a VH CDR1 of SEQ ID NO:101, a VH CDR2 of SEQ ID NO:102, and a VH CDR3 of SEQ ID NO:103, wherein the CDR is defined in accordance with the Kabat definition; and a light chain variable region that comprises a VL CDR1 of SEQ ID NO:111, a VL CDR2 of SEQ ID NO:112, and a VL CDR3 of SEQ ID NO:113, wherein the CDR is defined in accordance with the Kabat definition, and a modified heavy chain constant region comprising cysteine at positions 152 and 375, wherein said cysteine positions are numbered according to the EU system;
g. An antibody or antigen binding fragment thereof comprising a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at positions 152 and 375, wherein said cysteine positions are numbered according to the EU system;
h. An antibody or antigen binding fragment thereof comprising a heavy chain variable region (VH) comprising the amino acid sequence of SEQ ID NO:27, and a light chain variable region (VL) comprising the amino acid sequence of SEQ ID NO:37, and a modified heavy chain constant region comprising cysteine at positions 152 and 375, wherein said cysteine positions are numbered according to the EU system;
i. An antibody or antigen binding fragment thereof comprising a heavy chain variable region (VH) comprising the amino acid sequence of SEQ ID NO:47, and a light chain variable region (VL) comprising the amino acid sequence of SEQ ID NO:57, and a modified heavy chain constant region comprising cysteine at positions 152 and 375, wherein said cysteine positions are numbered according to the EU system;
j. An antibody or antigen binding fragment thereof comprising a heavy chain variable region (VH) comprising the amino acid sequence of SEQ ID NO:67, and a light chain variable region (VL) comprising the amino acid sequence of SEQ ID NO:77, and a modified heavy chain constant region comprising cysteine at positions 152 and 375, wherein said cysteine positions are numbered according to the EU system;
k. An antibody or antigen binding fragment thereof comprising a heavy chain variable region (VH) comprising the amino acid sequence of SEQ ID NO:87, and a light chain variable region (VL) comprising the amino acid sequence of SEQ ID NO:97, and a modified heavy chain constant region comprising cysteine at positions 152 and 375, wherein said cysteine positions are numbered according to the EU system;
l. An antibody or antigen binding fragment thereof comprising a heavy chain variable region (VH) comprising the amino acid sequence of SEQ ID NO:107, and a light chain variable region (VL) comprising the amino acid sequence of SEQ ID NO:117, and a modified heavy chain constant region comprising cysteine at positions 152 and 375, wherein said cysteine positions are numbered according to the EU system;
m. An antibody or antigen binding fragment thereof comprising a heavy chain comprising the amino acid sequence of SEQ ID NO:130, and a light chain comprising the amino acid sequence of SEQ ID NO:19;
n. An antibody or antigen binding fragment thereof comprising a heavy chain comprising the amino acid sequence of SEQ ID NO:133, and a light chain comprising the amino acid sequence of SEQ ID NO:39;
o. An antibody or antigen binding fragment thereof comprising a heavy chain comprising the amino acid sequence of SEQ ID NO:136, and a light chain comprising the amino acid sequence of SEQ ID NO:59;
p. An antibody or antigen binding fragment thereof comprising a heavy chain comprising the amino acid sequence of SEQ ID NO:139, and a light chain comprising the amino acid sequence of SEQ ID NO:79;
q. An antibody or antigen binding fragment thereof comprising a heavy chain comprising the amino acid sequence of SEQ ID NO:142, and a light chain comprising the amino acid sequence of SEQ ID NO:99;
r. An antibody or antigen binding fragment thereof comprising a heavy chain comprising the amino acid sequence of SEQ ID NO:145, and a light chain comprising the amino acid sequence of SEQ ID NO:119;
s. An antibody or antigen binding fragment thereof comprising a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
t. An antibody or antigen binding fragment thereof comprising a heavy chain variable region that comprises a VH CDR1 of SEQ ID NO: 21, a VH CDR2 of SEQ ID NO: 22, and a VH CDR3 of SEQ ID NO: 23, wherein the CDR is defined in accordance with the Kabat definition; and a light chain variable region that comprises a VL CDR1 of SEQ ID NO: 31, a VL CDR2 of SEQ ID NO: 32, and a VL CDR3 of SEQ ID NO: 33, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
u. An antibody or antigen binding fragment thereof comprising a heavy chain variable region that comprises a VH CDR1 of SEQ ID NO:41, a VH CDR2 of SEQ ID NO:42, and a VH CDR3 of SEQ ID NO:43, wherein the CDR is defined in accordance with the Kabat definition; and a light chain variable region that comprises a VL CDR1 of SEQ ID NO:51, a VL CDR2 of SEQ ID NO:52, and a VL CDR3 of SEQ ID NO:53, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
v. An antibody or antigen binding fragment thereof comprising a heavy chain variable region that comprises a VH CDR1 of SEQ ID NO:61, a VH CDR2 of SEQ ID NO:62, and a VH CDR3 of SEQ ID NO:63, wherein the CDR is defined in accordance with the Kabat definition; and a light chain variable region that comprises a VL CDR1 of SEQ ID NO:71, a VL CDR2 of SEQ ID NO:72, and a VL CDR3 of SEQ ID NO:73, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
w. An antibody or antigen binding fragment thereof comprising a heavy chain variable region that comprises a VH CDR1 of SEQ ID NO:81, a VH CDR2 of SEQ ID NO:82, and a VH CDR3 of SEQ ID NO:83, wherein the CDR is defined in accordance with the Kabat definition; and a light chain variable region that comprises a VL CDR1 of SEQ ID NO:91, a VL CDR2 of SEQ ID NO:92, and a VL CDR3 of SEQ ID NO:93, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
x. An antibody or antigen binding fragment thereof comprising a heavy chain variable region that comprises a VH CDR1 of SEQ ID NO:101, a VH CDR2 of SEQ ID NO:102, and a VH CDR3 of SEQ ID NO:103, wherein the CDR is defined in accordance with the Kabat definition; and a light chain variable region that comprises a VL CDR1 of SEQ ID NO:111, a VL CDR2 of SEQ ID NO:112, and a VL CDR3 of SEQ ID NO:113, wherein the CDR is defined in accordance with the Kabat definition, and a modified heavy chain constant region comprising cysteine at position 360, and a modified light chain constant region comprising cysteine at position 107, wherein said cysteine positions are numbered according to the EU system;
y. An antibody or antigen binding fragment thereof comprising a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at position 360, and a modified light chain constant region comprising cysteine at position 107, wherein said cysteine positions are numbered according to the EU system;
z. An antibody or antigen binding fragment thereof comprising a heavy chain variable region (VH) comprising the amino acid sequence of SEQ ID NO:27, and a light chain variable region (VL) comprising the amino acid sequence of SEQ ID NO:37, and a modified heavy chain constant region comprising cysteine at position 360, and a modified light chain constant region comprising cysteine at position 107, wherein said cysteine positions are numbered according to the EU system;
aa. An antibody or antigen binding fragment thereof comprising a heavy chain variable region (VH) comprising the amino acid sequence of SEQ ID NO:47, and a light chain variable region (VL) comprising the amino acid sequence of SEQ ID NO:57, and a modified heavy chain constant region comprising cysteine at position 360, and a modified light chain constant region comprising cysteine at position 107, wherein said cysteine positions are numbered according to the EU system;
bb. An antibody or antigen binding fragment thereof comprising a heavy chain variable region (VH) comprising the amino acid sequence of SEQ ID NO:67, and a light chain variable region (VL) comprising the amino acid sequence of SEQ ID NO:77, and a modified heavy chain constant region comprising cysteine at position 360, and a modified light chain constant region comprising cysteine at position 107, wherein said cysteine positions are numbered according to the EU system;
cc. An antibody or antigen binding fragment thereof comprising a heavy chain variable region (VH) comprising the amino acid sequence of SEQ ID NO:87, and a light chain variable region (VL) comprising the amino acid sequence of SEQ ID NO:97, and a modified heavy chain constant region comprising cysteine at position 360, and a modified light chain constant region comprising cysteine at position 107, wherein said cysteine positions are numbered according to the EU system;
dd. An antibody or antigen binding fragment thereof comprising a heavy chain variable region (VH) comprising the amino acid sequence of SEQ ID NO:107, and a light chain variable region (VL) comprising the amino acid sequence of SEQ ID NO:117and a modified heavy chain constant region comprising cysteine at position 360, and a modified light chain constant region comprising cysteine at position 107, wherein said cysteine positions are numbered according to the EU system;
ee. An antibody or antigen binding fragment thereof comprising a heavy chain comprising the amino acid sequence of SEQ ID NO:131, and a light chain comprising the amino acid sequence of SEQ ID NO:132;
ff. An antibody or antigen binding fragment thereof comprising a heavy chain comprising the amino acid sequence of SEQ ID NO:134, and a light chain comprising the amino acid sequence of SEQ ID NO:135;
gg. An antibody or antigen binding fragment thereof comprising a heavy chain comprising the amino acid sequence of SEQ ID NO:137, and a light chain comprising the amino acid sequence of SEQ ID NO:138;
hh. An antibody or antigen binding fragment thereof comprising a heavy chain comprising the amino acid sequence of SEQ ID NO:140, and a light chain comprising the amino acid sequence of SEQ ID NO:141;
ii. An antibody or antigen binding fragment thereof comprising a heavy chain comprising the amino acid sequence of SEQ ID NO:143, and a light chain comprising the amino acid sequence of SEQ ID NO:144; or
jj. An antibody or antigen binding fragment thereof comprising a heavy chain comprising the amino acid sequence of SEQ ID NO:146, and a light chain comprising the amino acid sequence of SEQ ID NO:147.

2. An antibody drug conjugate comprising a formula selected from:
(Formula A) or ((D)_{z})-L)_{y}-Ab (Formula B)
or a pharmaceutically acceptable salt thereof, wherein:
Ab is an antibody or antigen binding fragment thereof according to claim 1;
z is an integer from 1 to 8;
y is an integer from 1 to 16;
L is a linker; and
D is a drug moiety.

3. (canceled)

4. The antibody drug conjugate of claim 2, wherein Ab is conjugated to L via a thiol-maleimide linkage at the cysteine residues at positions 152 and 375 of the heavy chain constant region of the antibody, wherein said cysteine positions are numbered according to the EU system.

5. The antibody drug conjugate of claim 2, wherein Ab is conjugated to L via a thiol-maleimide linkage at the cysteine residue at position 360 of the heavy chain constant region of the antibody and position 107 of the light chain constant region, wherein said cysteine positions are numbered according to the EU system.

6. The antibody drug conjugate of claim 2, wherein the antibody or antigen binding fragment thereof is conjugated to L via an oxime linkage at one or more interchain disulfide bridges of the antibody.

7. (canceled)

8. (canceled)

9. The antibody drug conjugate of claim 2 comprising a structure selected from:

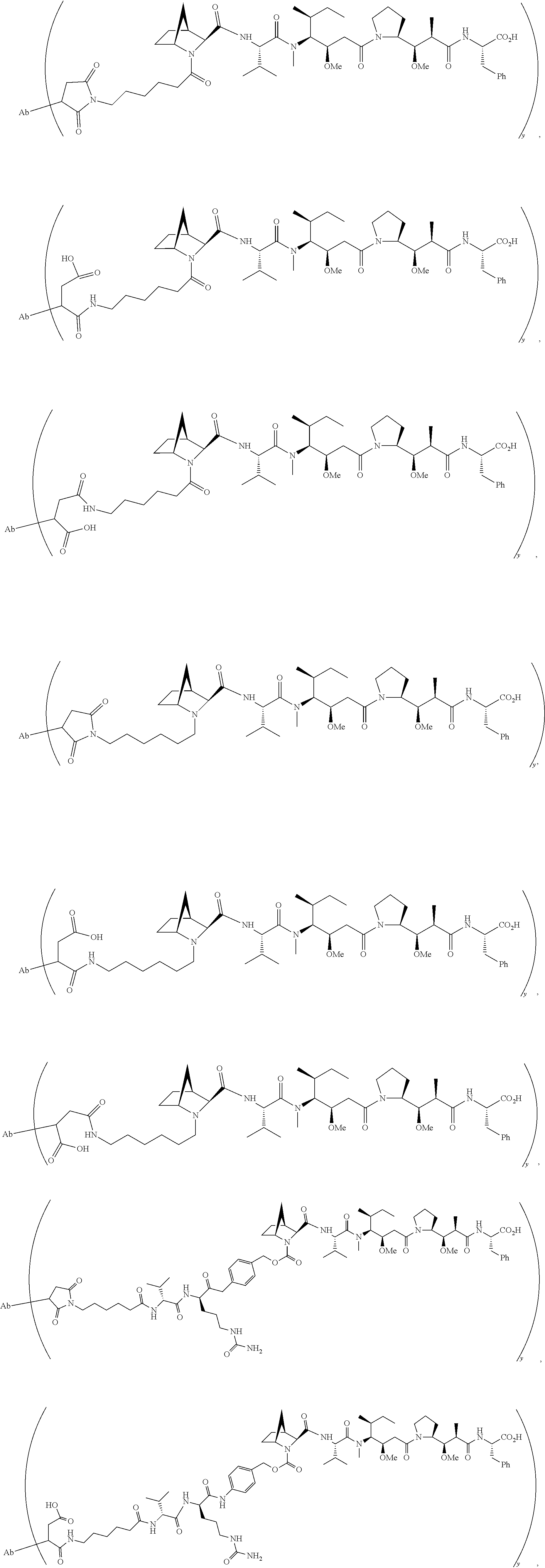

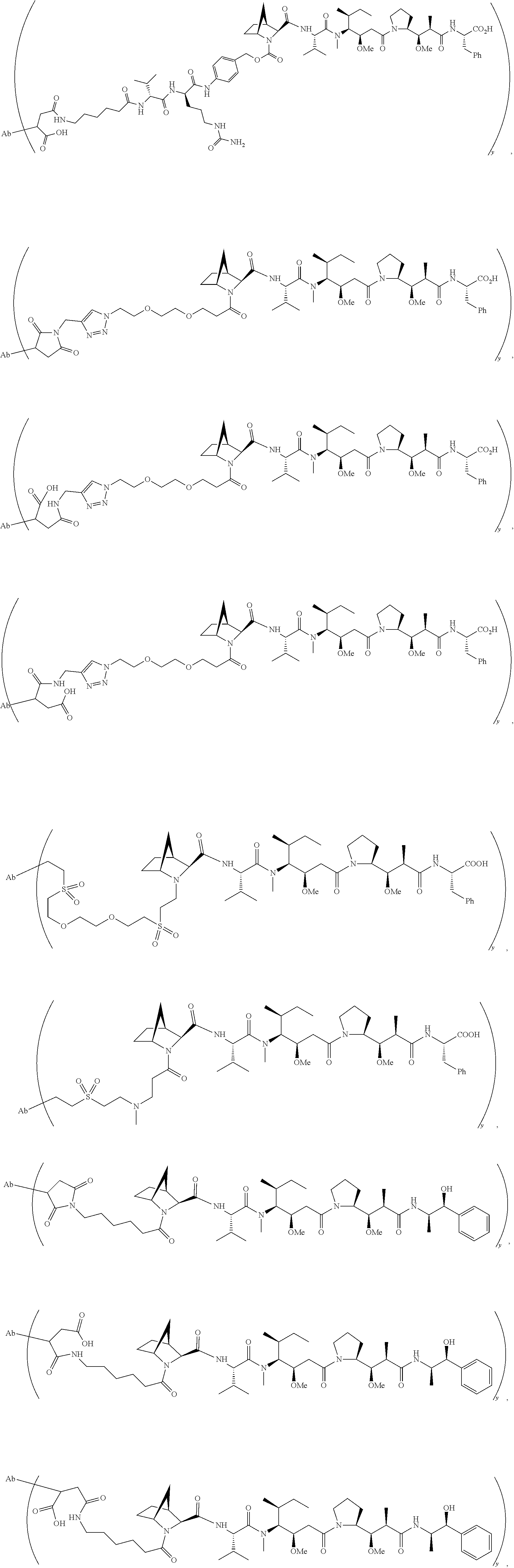

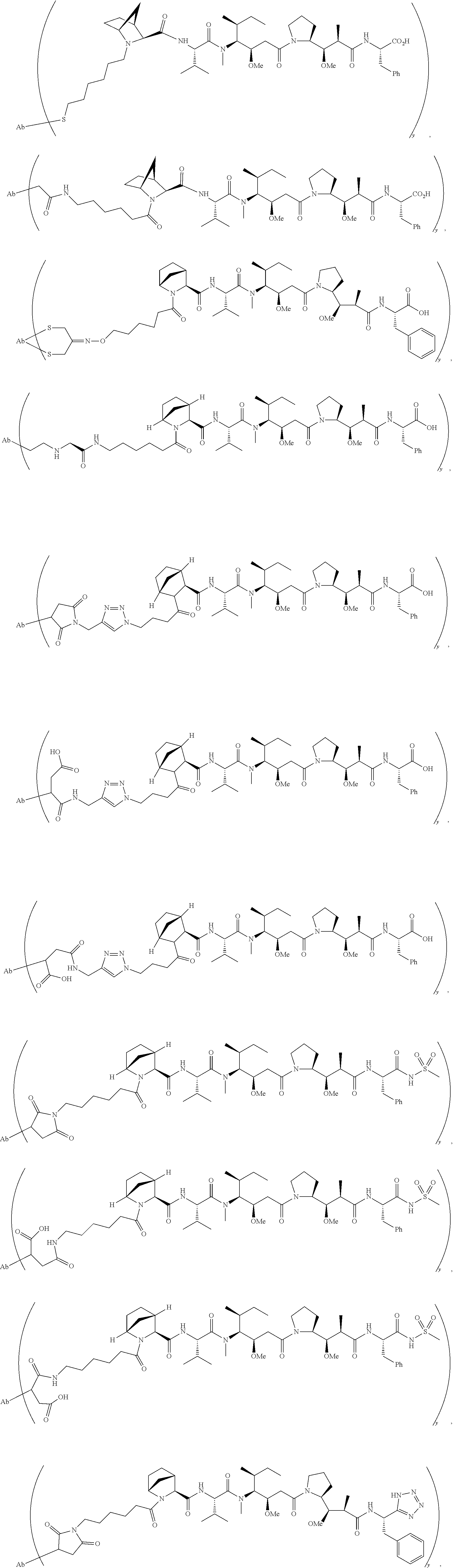

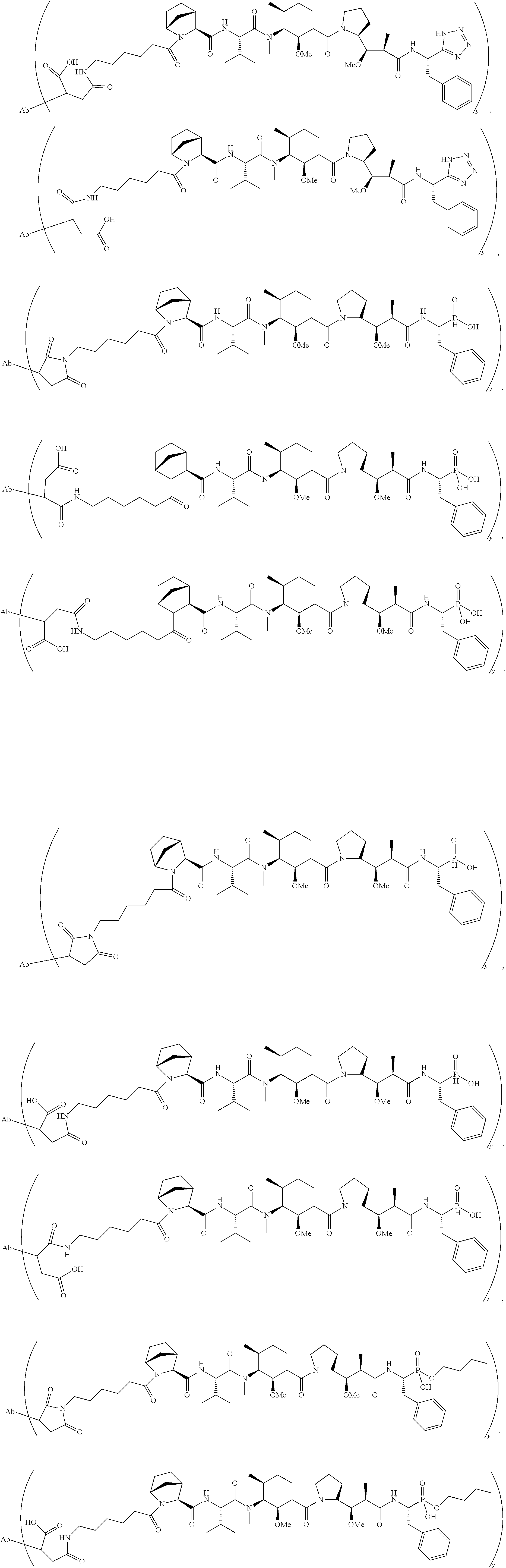

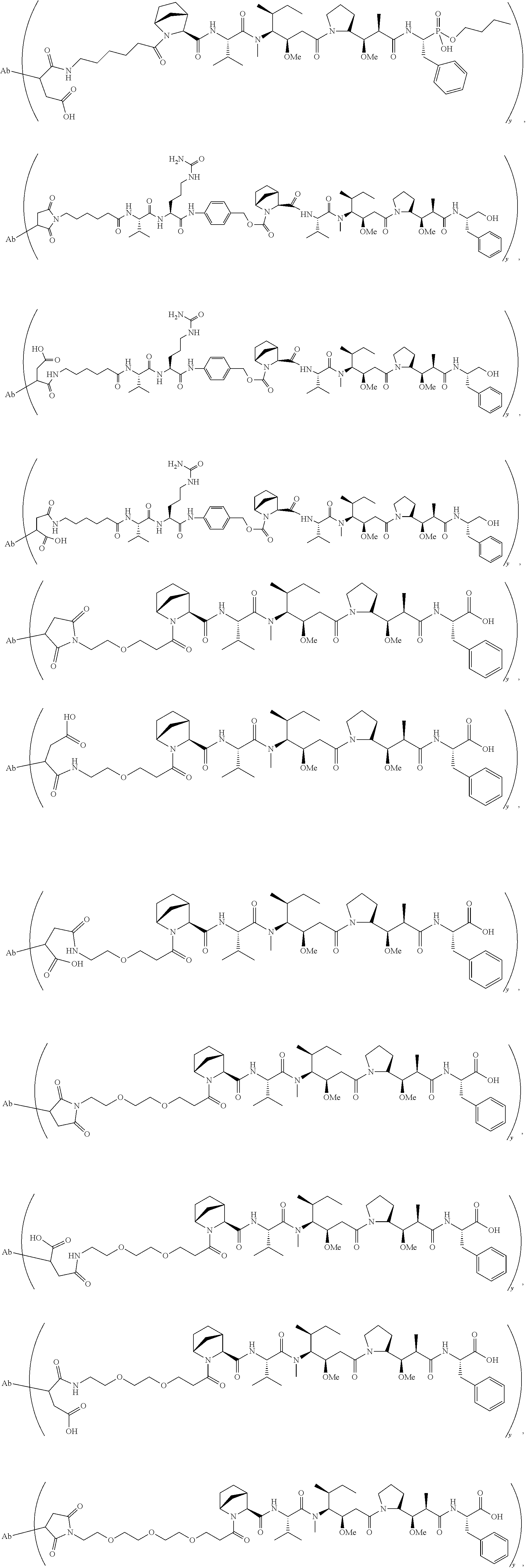

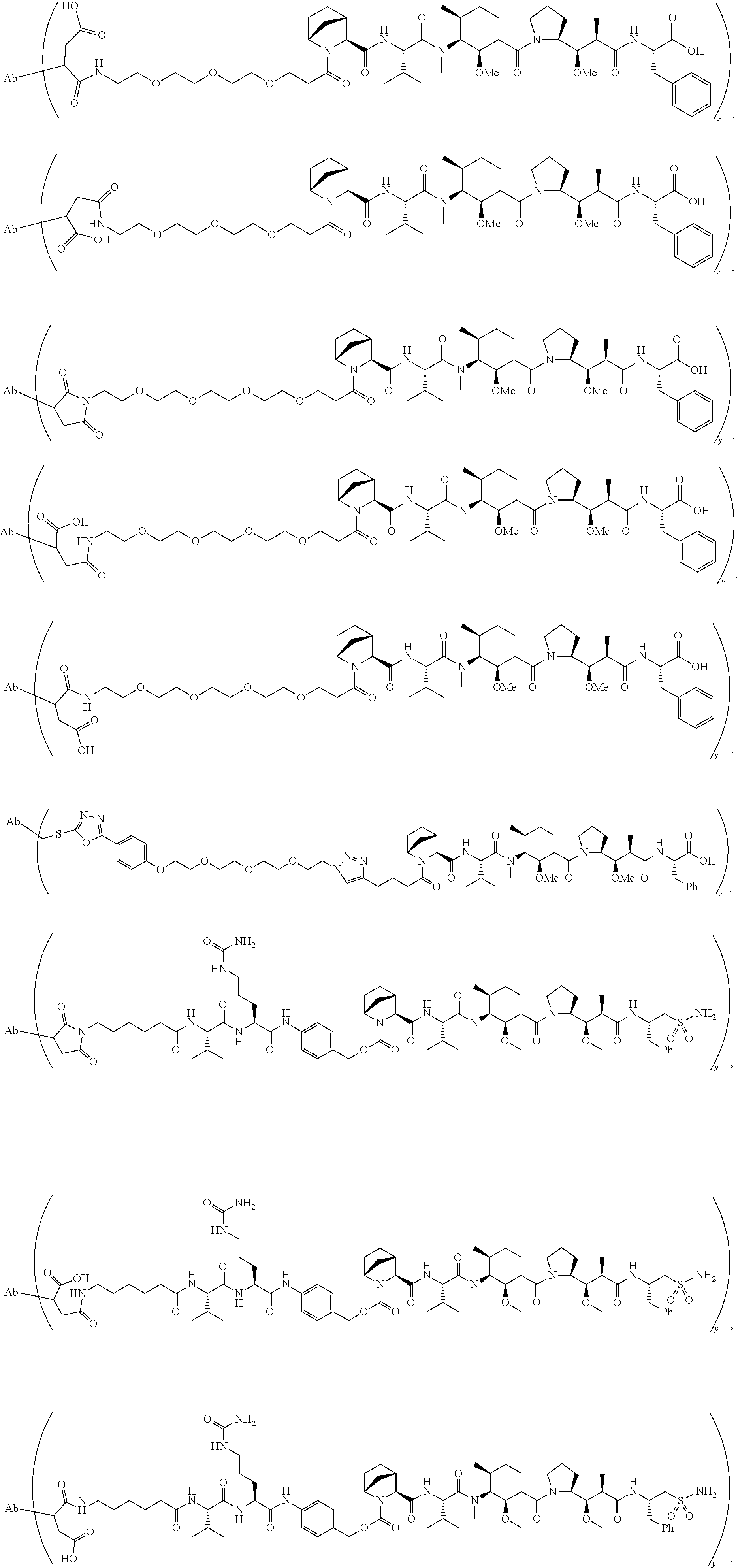

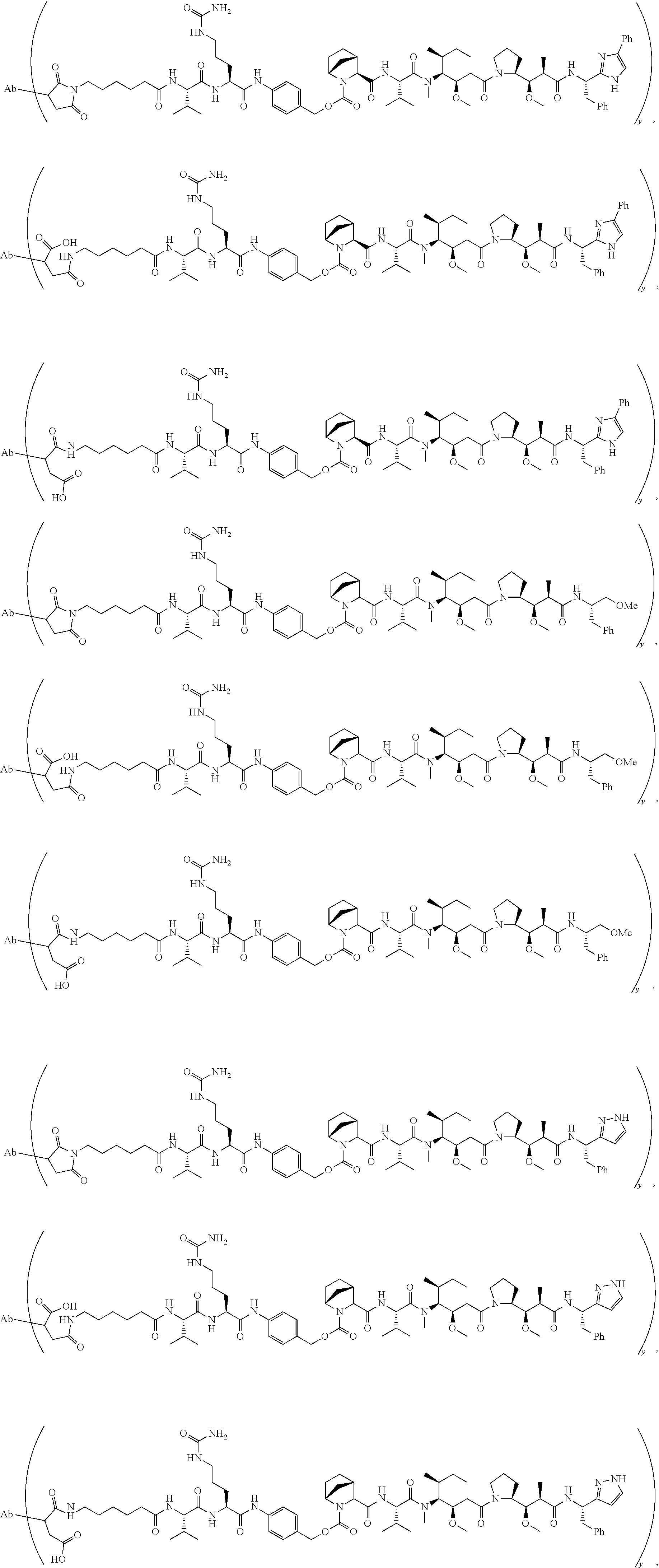

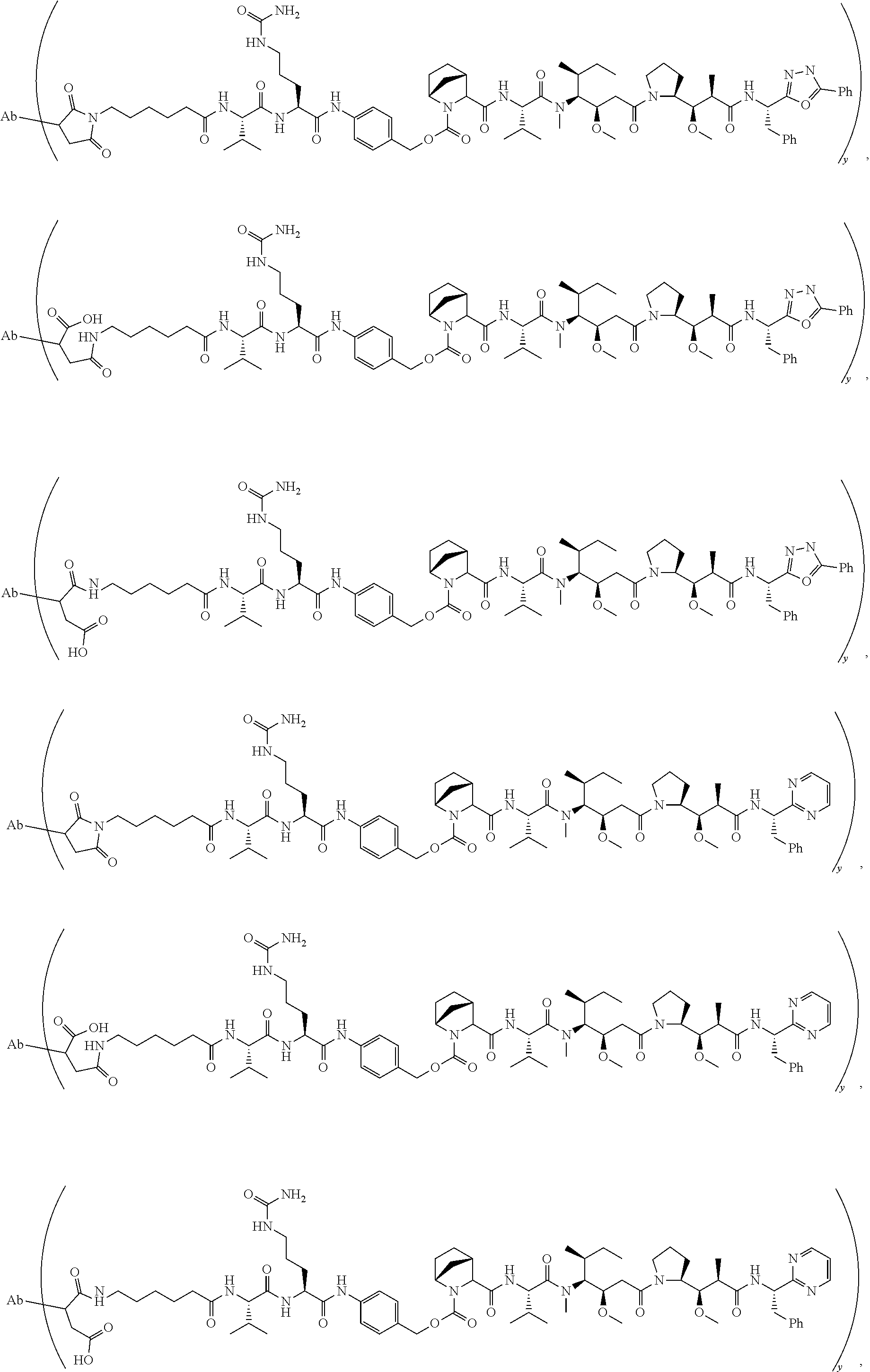

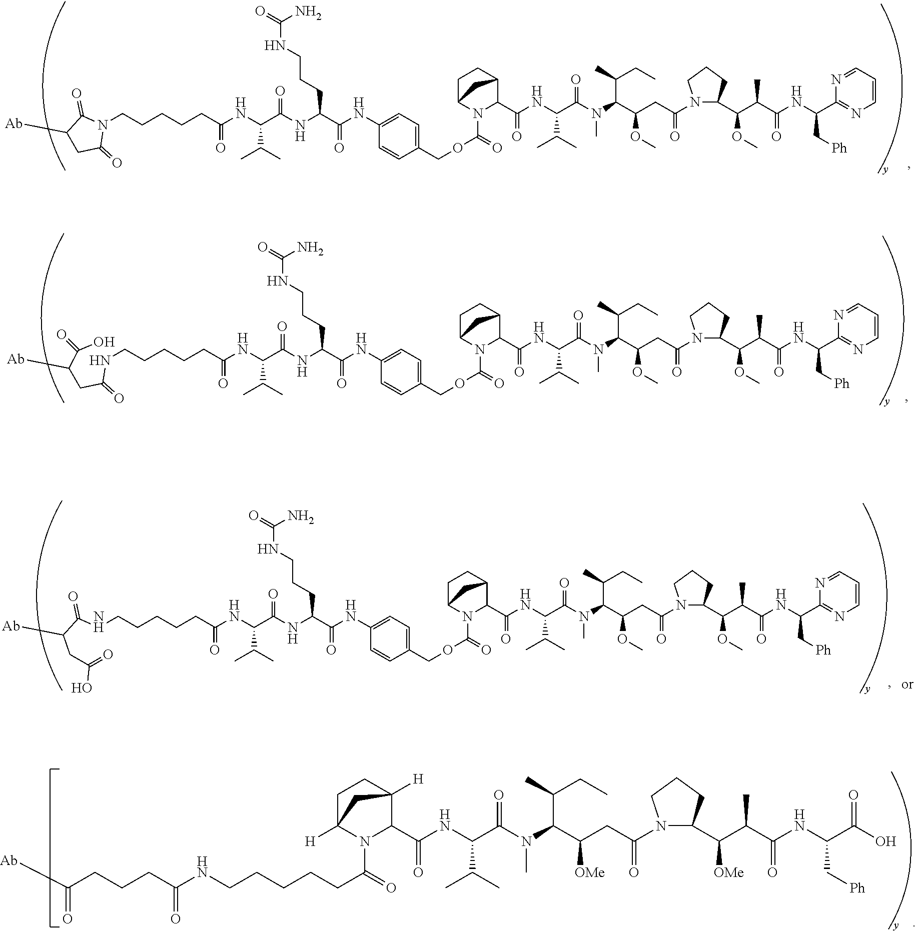

10. The antibody drug conjugate of claim 2 comprising a structure selected from:

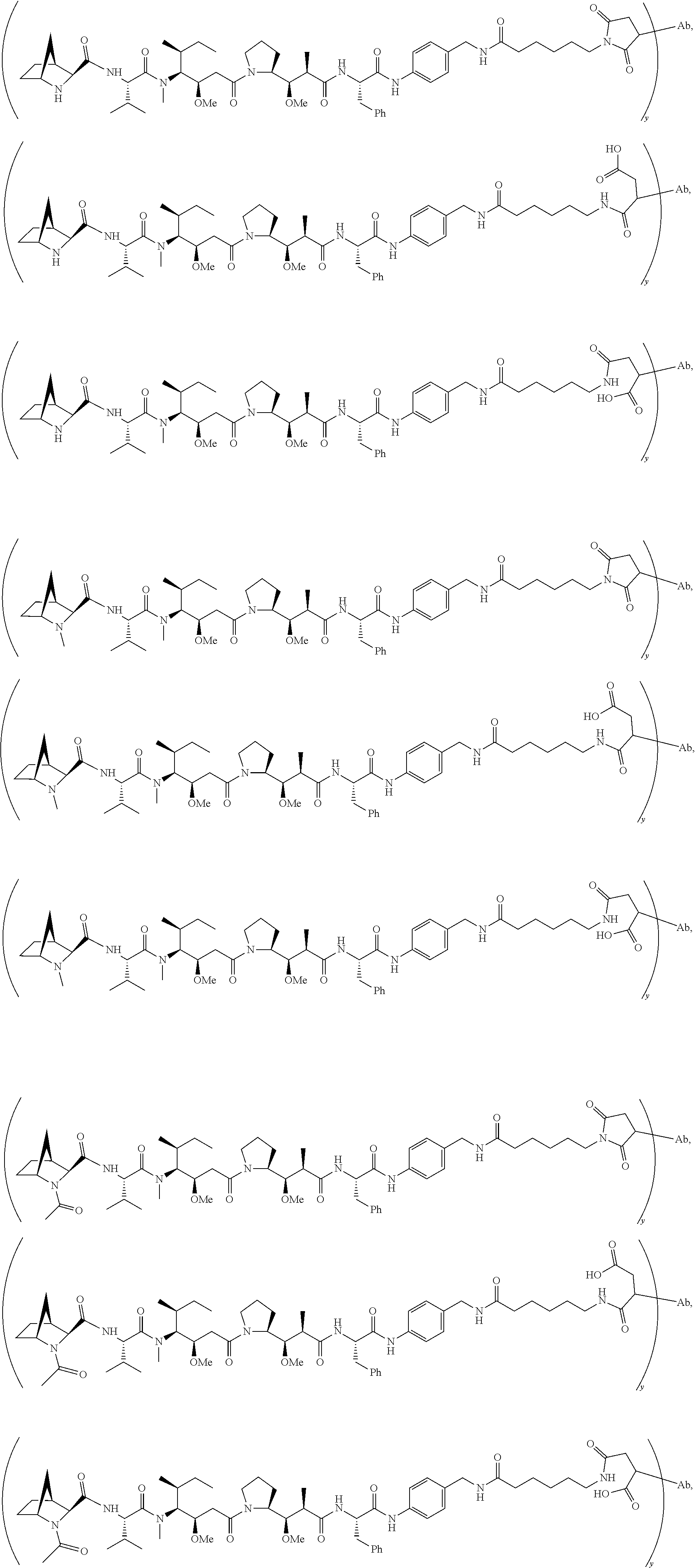

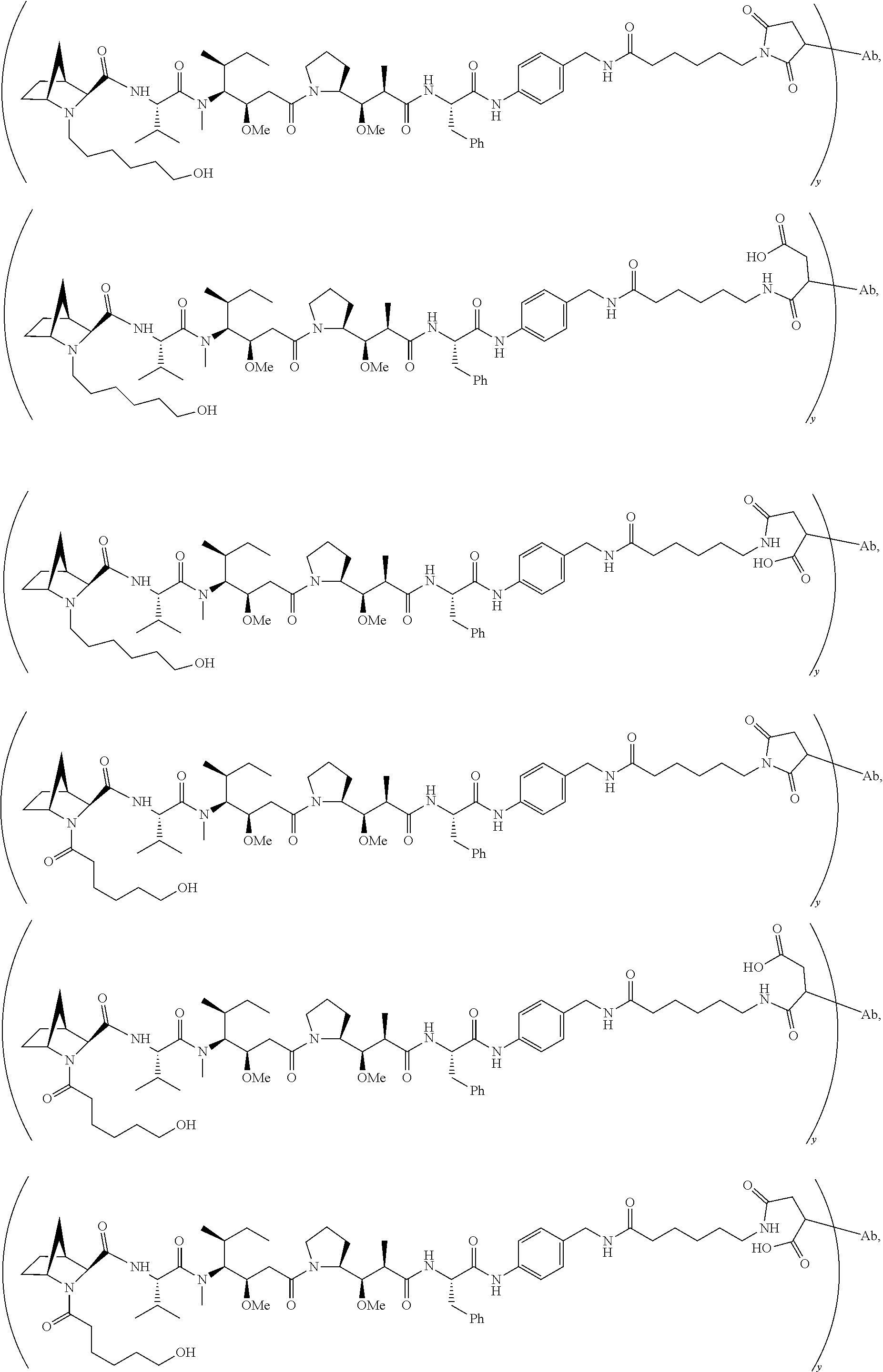

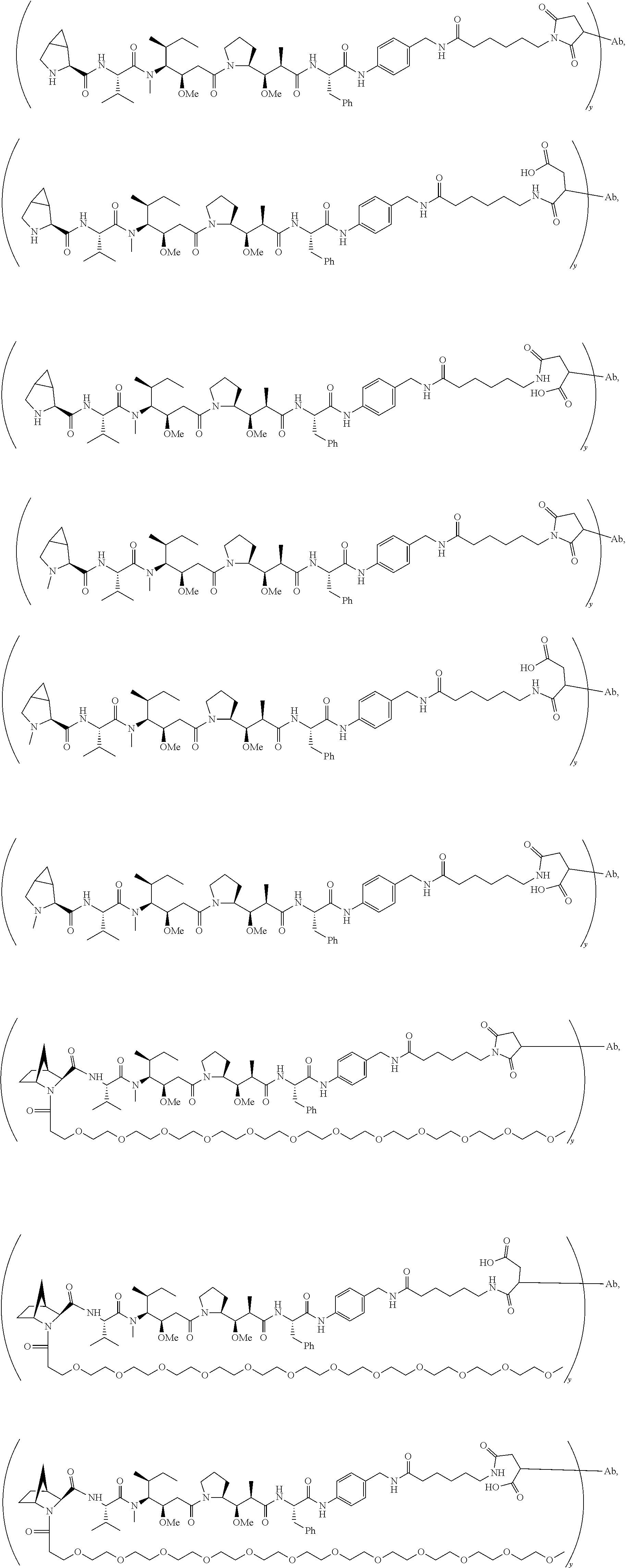

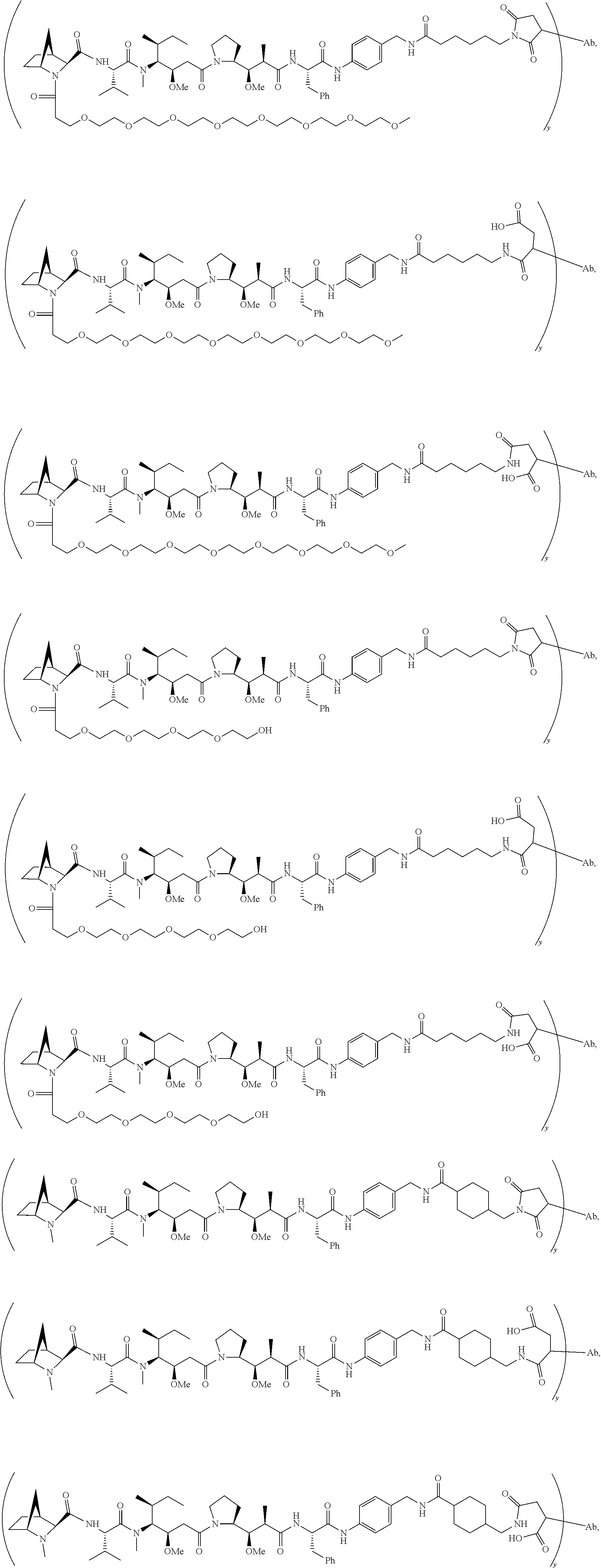

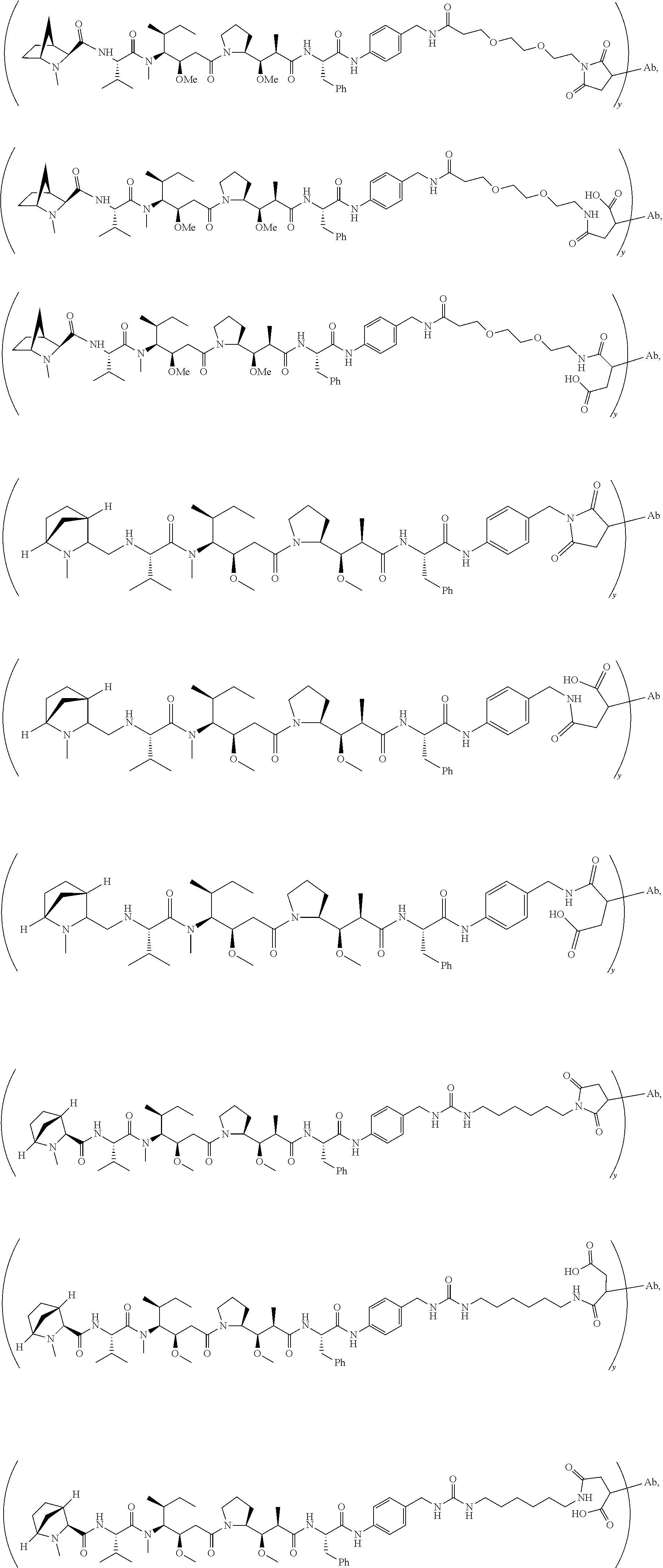

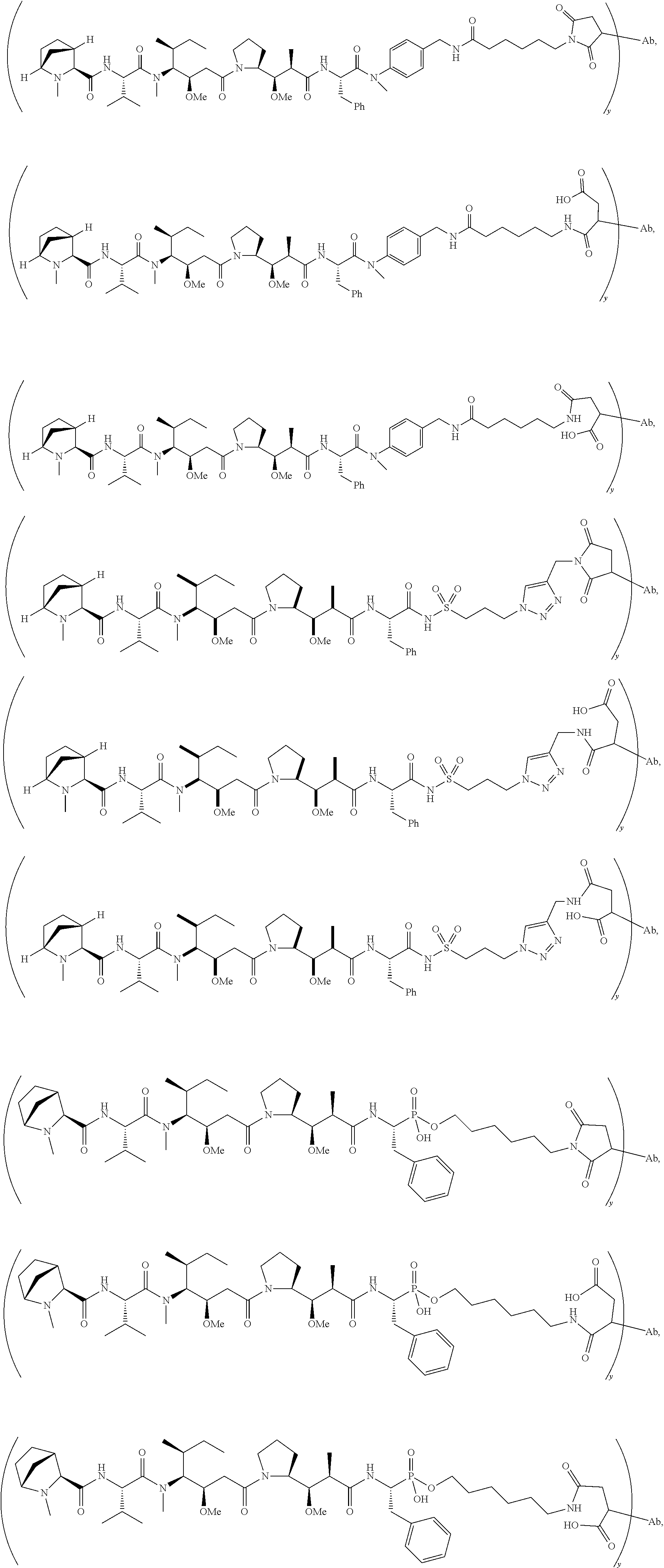

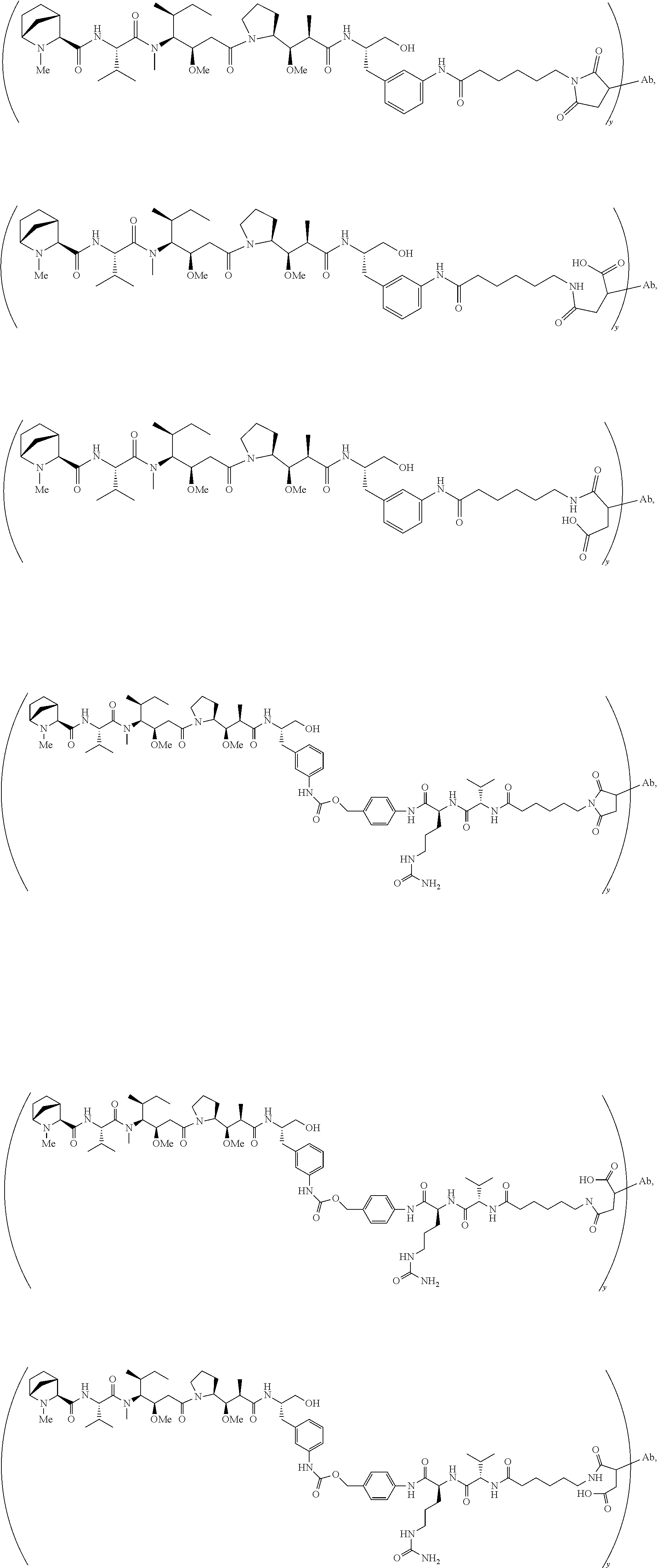

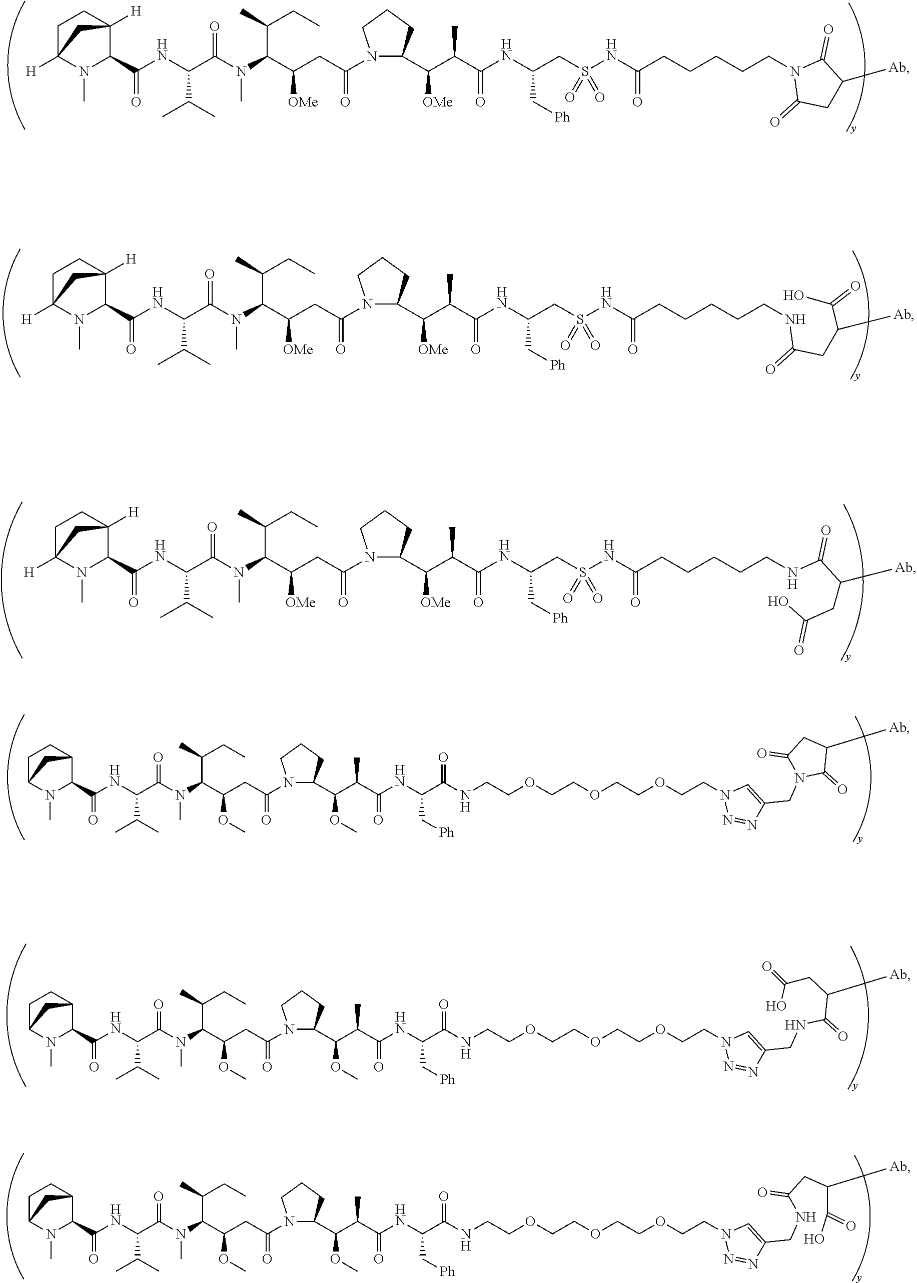

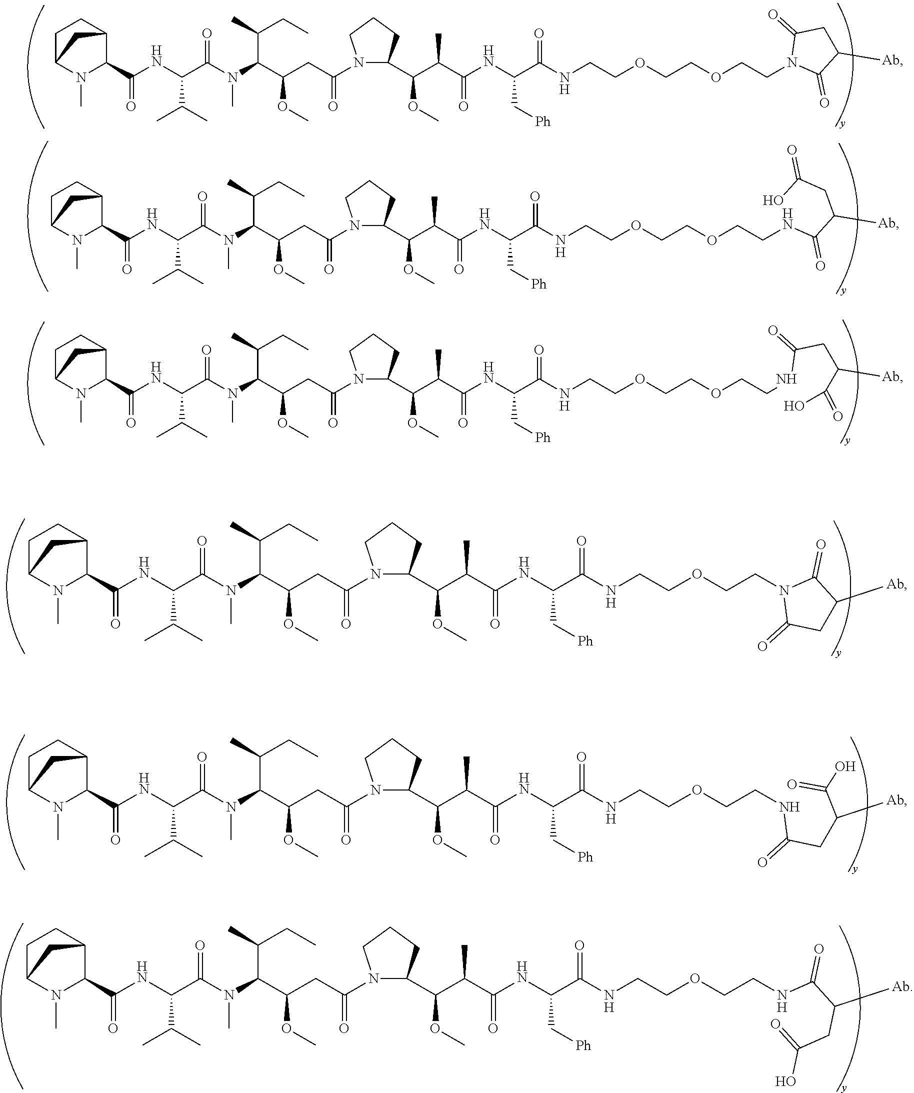

11. The antibody drug conjugate of claim 2, wherein the structure is selected from:

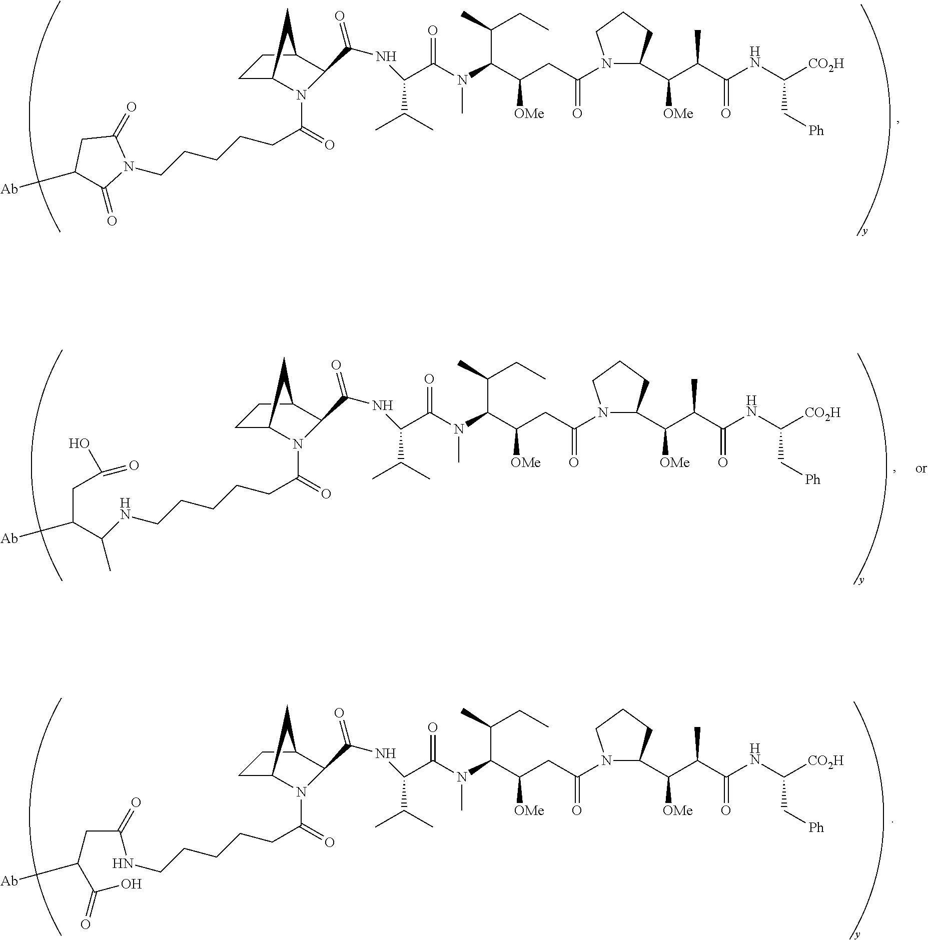

12. The antibody drug conjugate of claim 2, wherein the structure is selected from:

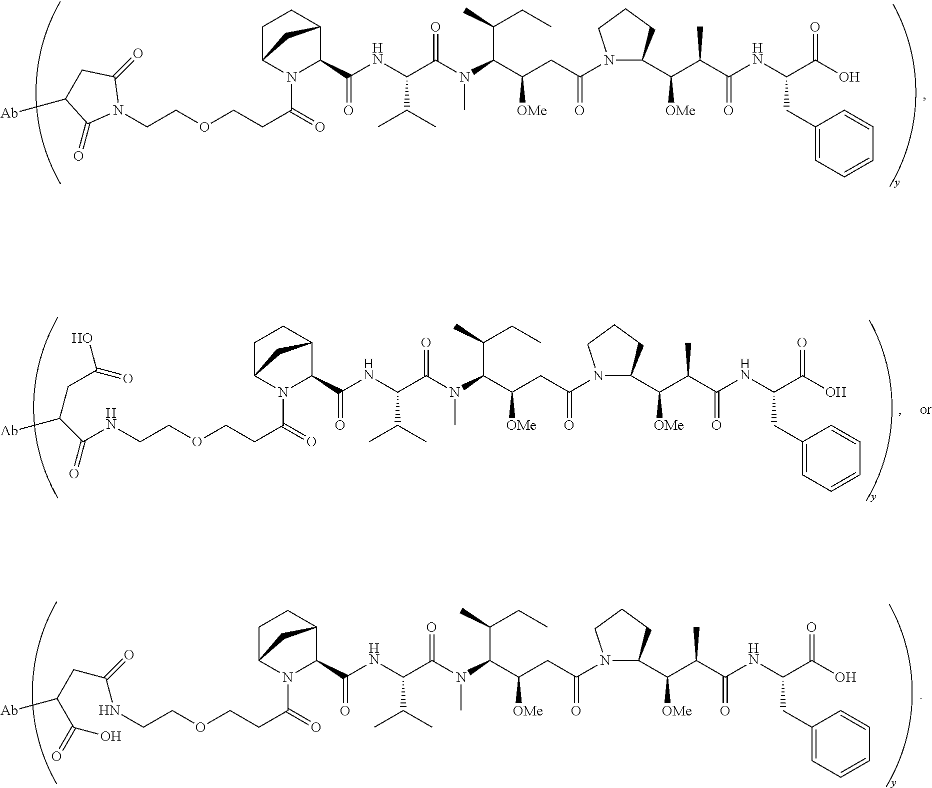

13. The antibody drug conjugate of claim 2, comprising the structure:

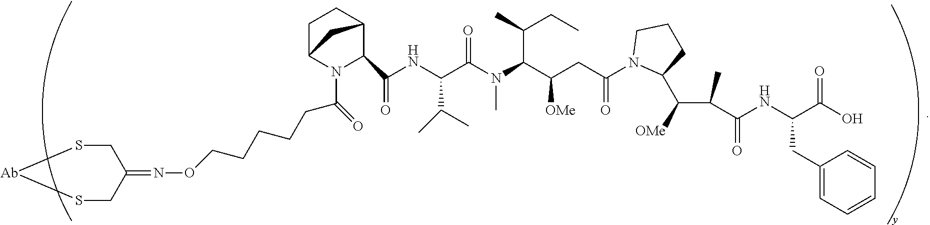

14. The antibody drug conjugate of claim 2, wherein the antibody or antigen binding fragment thereof comprises a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition, and a modified heavy chain constant region comprising cysteine at positions 152 and 375, wherein said cysteine positions are numbered according to the EU system.

15. The antibody drug conjugate of claim 2, wherein the antibody or antigen binding fragment thereof comprises a heavy chain variable region (VH) comprising the amino acid sequence of SEQ ID NO:7, and a light chain variable region (VL) comprising the amino acid sequence of SEQ ID NO:17, and a modified heavy chain constant region comprising cysteine at positions 152 and 375, wherein said cysteine positions are numbered according to the EU system.

16. The antibody drug conjugate of claim 2, wherein the antibody or antigen binding fragment thereof comprises a heavy chain comprising the amino acid sequence of SEQ ID NO:130, and a light chain comprising the amino acid sequence of 19.

17. The antibody drug conjugate of claim 13, wherein the antibody or antigen binding fragment thereof comprises a heavy chain variable region that comprises a VH CDR1 of SEQ ID NO: 1, a VH CDR2 of SEQ ID NO: 2, and a VH CDR3 of SEQ ID NO: 3, wherein the CDR is defined in accordance with the Kabat definition; and a light chain variable region that comprises a VL CDR1 of SEQ ID NO: 11, a VL CDR2 of SEQ ID NO: 12, and a VL CDR3 of SEQ ID NO: 13, wherein the CDR is defined in accordance with the Kabat definition.

18. The antibody drug conjugate of claim 13, wherein the antibody or antigen binding fragment thereof comprises a heavy chain variable region (VH) comprising the amino acid sequence of SEQ ID NO:7, and a light chain variable region (VL) comprising the amino acid sequence of SEQ ID NO:17.

19. The antibody drug conjugate of claim 13, wherein the antibody or antigen binding fragment thereof comprises a heavy chain comprising the amino acid sequence of SEQ ID NO:9, and a light chain comprising the amino acid sequence of 19.

20. An antibody drug conjugate having a structure Ab(-L-D)y selected from: 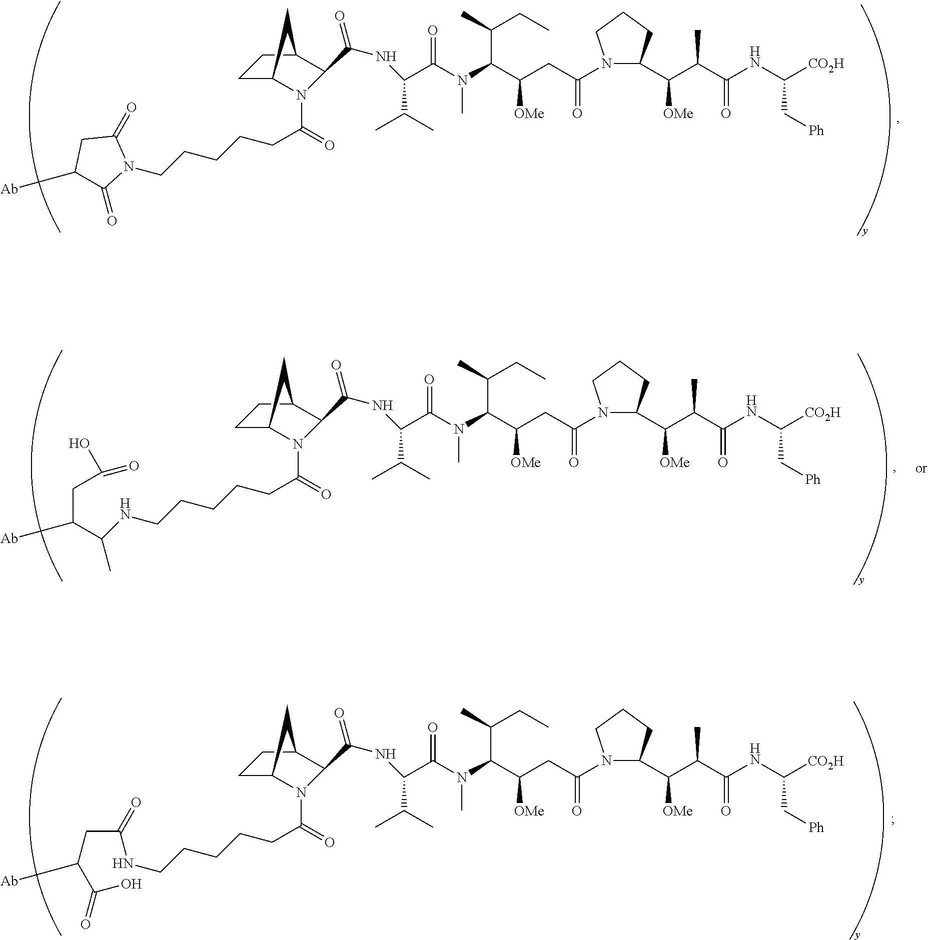
wherein Ab is an antibody comprising a heavy chain having the amino acid sequence of SEQ ID NO:130, and a light chain comprising the amino acid sequence of SEQ ID NO:19, wherein the L-D is conjugated to the Ab via maleimide linkage at the cysteine residues at positions 158 and 381 of SEQ ID NO 130, and wherein y is 4.

21. An antibody drug conjugate having the structure: 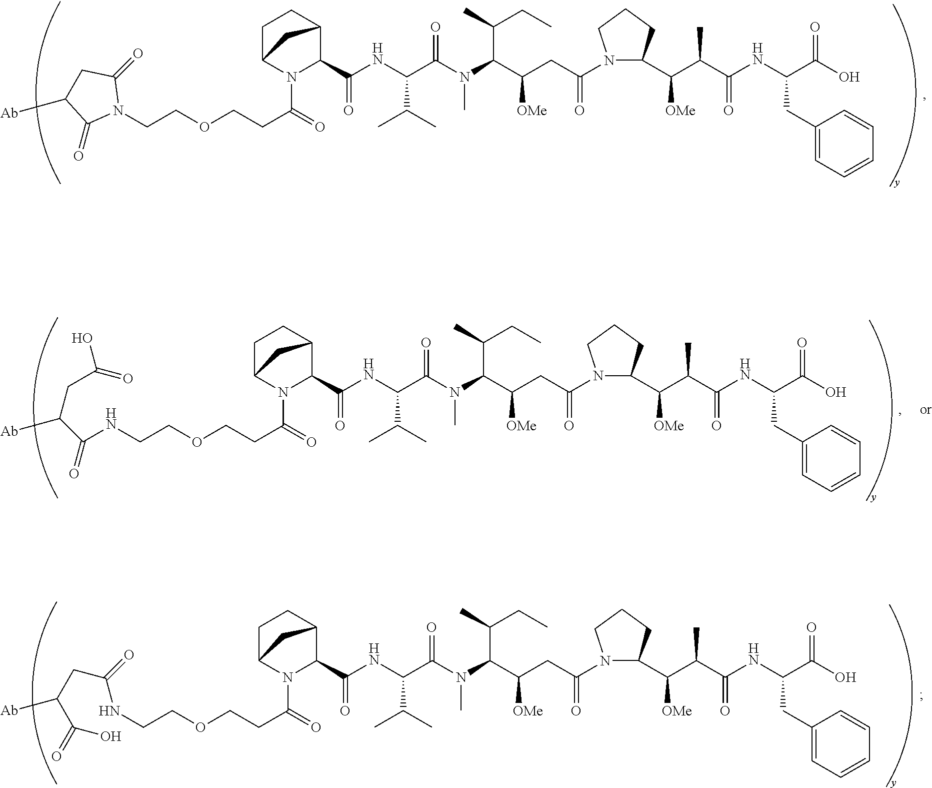
wherein Ab is an antibody comprising a heavy chain having the amino acid sequence of SEQ ID NO:130, and a light chain comprising the amino acid sequence of SEQ ID NO:19, wherein the linker-payload [this term appears to lack antecedent basis] is conjugated to the Ab via maleimide linkage at the cysteine residues at positions 158 and 381 of SEQ ID NO 130, and wherein y is 4.

22. An antibody drug conjugate having the structure: 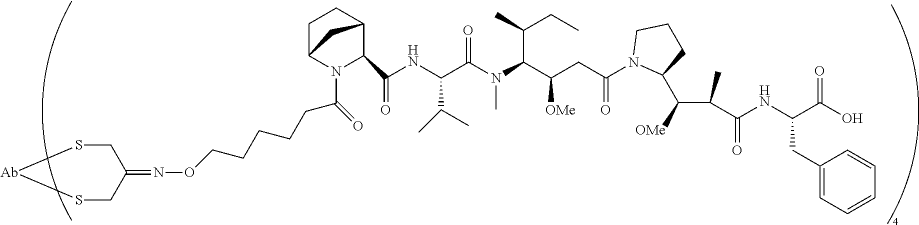
wherein Ab is an antibody comprising a heavy chain having the amino acid sequence of SEQ ID NO:9, and a light chain having the amino acid sequence of SEQ ID NO:19; and wherein the linker payload [above] is conjugated to the Ab at the interchain disulfide bonds of the Ab.

23. The antibody drug conjugate of claim 2, wherein said z is 1.

24. The antibody drug conjugate of claim 2, wherein said y is 4.

25. (canceled)

26. A pharmaceutical composition comprising the antibody drug conjugate of claim 2 and a pharmaceutically acceptable carrier.

27. The pharmaceutical composition of claim 26 wherein said composition is prepared as a lyophilisate.

28. A method of treating cancer in a patient in need thereof, comprising administering to said patient the antibody drug conjugate of claim 2.

29. The method of claim 28, wherein the antibody drug conjugate is administered to the patient in combination with one or more additional therapeutic compounds.

30. (canceled)

31. (canceled)

32. (canceled)

33. (canceled)

34. The method of claim 28, wherein the cancer expresses P-cadherin.

35. The method of claim 34, wherein the cancer is selected from the group consisting of adrenocortical carcinoma, bladder cancer, bone cancer, breast cancer, central nervous system atypical teratoid/rhabdoid tumors, colon cancer, colorectal cancer, embryonal tumors, endometrial cancer, esophageal cancer, gastric cancer, head and neck cancer, hepatocellular cancer, Kaposi sarcoma, liver cancer, lung cancer, including small cell lung cancer and non-small cell lung cancer, ovarian cancer, rectal cancer, rhabdomyosarcomasmall intestine cancer, soft tissue sarcoma, squamous cell carcinoma, squamous neck cancer, stomach cancer, uterine cancer, vaginal cancer, and vulvar canceradrenocortical carcinoma, bladder cancer, bone cancer, breast cancer, central nervous system atypical teratoid/rhabdoid tumors, colon cancer, colorectal cancer, embryonal tumors, endometrial cancer, esophageal cancer, gastric cancer, head and neck cancer, hepatocellular cancer, Kaposi sarcoma, liver cancer, lung cancer, including small cell lung cancer and non-small cell lung cancer, ovarian cancer, rectal cancer, rhabdomyosarcomasmall intestine cancer, soft tissue sarcoma, squamous cell carcinoma, squamous neck cancer, stomach cancer, uterine cancer, vaginal cancer, and vulvar cancer.

36. (canceled)

37. (canceled)

38. (canceled)

39. (canceled)

40. A nucleic acid that encodes the antibody or antigen binding fragment of claim 1.

41. A vector comprising the nucleic acid of claim 40.

42. A host cell comprising the vector according to claim 41.

43. A process for producing an antibody or antigen binding fragment comprising cultivating the host cell of claim 42 and recovering the antibody from the culture.

44. A diagnostic reagent comprising the antibody or antigen binding fragment thereof of claim 1.

45. The diagnostic reagent of claim 44, wherein the antibody or antigen binding fragment thereof is labeled with a radiolabel, a fluorophore, a chromophore, an imaging agent, or a metal ion.

46. (canceled)

47. (canceled)

48. (canceled)


SEQUENCE LISTING (168 sequences)

SEQ ID NO: 1 - protein, 7 aa, Artificial Sequence
  source: /note=“Description of Artificial Sequence Synthetic peptide”
SQSAAWN

SEQ ID NO: 2 - protein, 18 aa, Artificial Sequence
  source: /note=“Description of Artificial Sequence Synthetic peptide”
RIYYRSKWYNDYALSVKS

SEQ ID NO: 3 - protein, 11 aa, Artificial Sequence
  source: /note=“Description of Artificial Sequence Synthetic peptide”
GEGYGREGFAI

SEQ ID NO: 4 - protein, 9 aa, Artificial Sequence
  source: /note=“Description of Artificial Sequence Synthetic peptide”
GDSVSSQSA

SEQ ID NO: 5 - protein, 7 aa, Artificial Sequence
  source: /note=“Description of Artificial Sequence Synthetic peptide”
YYRSKWY

SEQ ID NO: 6 - protein, 11 aa, Artificial Sequence
  source: /note=“Description of Artificial Sequence Synthetic peptide”
GEGYGREGFAI

SEQ ID NO: 7 - protein, 123 aa, Artificial Sequence
  source: /note=“Description of Artificial Sequence Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSS

SEQ ID NO: 8 - DNA, 369 bp, Artificial Sequence
  source: /note=“Description of Artificial Sequence Synthetic polynucleotide”
caggtgcagctgcagcagtcaggccctggcctggtcaagcctagtcagaccctgagcctgacctgcgctattagcggcgatagtgtgtctagtcagtcagccgcctggaactggattagacagtcaccctctaggggcctggagtggctgggtagaatctactataggtctaagtggtataacgactacgccctgagcgtgaagtctaggatcactattaaccccgacacctctaagaatcagtttagcctgcagctgaatagcgtgacccccgaggacaccgccgtctactactgcgctagaggcgagggctacggtagagagggcttcgctatctggggtcagggcaccctggtcaccgtgtctagc

SEQ ID NO: 9 - protein, 453 aa, Artificial Sequence
  source: /note=“Description of Artificial Sequence Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1359 bp, Artificial Sequence
  source: /note=“Description of Artificial Sequence Synthetic polynucleotide”
caggtgcagctgcagcagtcaggccctggcctggtcaagcctagtcagaccctgagcctgacctgcgctattagcggcgatagtgtgtctagtcagtcagccgcctggaactggattagacagtcaccctctaggggcctggagtggctgggtagaatctactataggtctaagtggtataacgactacgccctgagcgtgaagtctaggatcactattaaccccgacacctctaagaatcagtttagcctgcagctgaatagcgtgacccccgaggacaccgccgtctactactgcgctagaggcgagggctacggtagagagggcttcgctatctggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 11 - protein, 11 aa, Artificial Sequence
  source: /note=“Description of Artificial Sequence Synthetic peptide”
RASQTISNTLA

SEQ ID NO: 12 - protein, 7 aa, Artificial Sequence
  source: /note=“Description of Artificial Sequence Synthetic peptide”
AASNLQS

SEQ ID NO: 13 - protein, 8 aa, Artificial Sequence
  source: /note=“Description of Artificial Sequence Synthetic peptide”
QQYLSWFT

SEQ ID NO: 14 - protein, 7 aa, Artificial Sequence
  source: /note=“Description of Artificial Sequence Synthetic peptide”
SQTISNT

SEQ ID NO: 15 - protein, 3 aa, Artificial Sequence
  source: /note=“Description of Artificial Sequence Synthetic peptide”
AAS

SEQ ID NO: 16 - protein, 5 aa, Artificial Sequence
  source: /note=“Description of Artificial Sequence Synthetic peptide”
YLSWF

SEQ ID NO: 17 - protein, 106 aa, Artificial Sequence
  source: /note=“Description of Artificial Sequence Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIK

SEQ ID NO: 18 - DNA, 318 bp, Artificial Sequence
  source: /note=“Description of Artificial Sequence Synthetic polynucleotide”
gatattcagatgactcagtcacctagtagcctgagcgctagtgtgggcgatagagtgactatcacctgtagagcctctcagactatctctaacaccctggcctggtatcagcagaagcccggtaaagcccctaagctgctgatctacgccgcctctaacctgcagtcaggcgtgccctctaggtttagcggtagcggtagtggcaccgacttcaccctgactattagtagcctgcagcccgaggacttcgctacctactactgtcagcagtacctgagctggttcaccttcggtcagggcactaaggtcgagattaag

SEQ ID NO: 19 - protein, 213 aa, Artificial Sequence
  source: /note=“Description of Artificial Sequence Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 639 bp, Artificial Sequence
  source: /note=“Description of Artificial Sequence Synthetic polynucleotide”
gatattcagatgactcagtcacctagtagcctgagcgctagtgtgggcgatagagtgactatcacctgtagagcctctcagactatctctaacaccctggcctggtatcagcagaagcccggtaaagcccctaagctgctgatctacgccgcctctaacctgcagtcaggcgtgccctctaggtttagcggtagcggtagtggcaccgacttcaccctgactattagtagcctgcagcccgaggacttcgctacctactactgtcagcagtacctgagctggttcaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 21 - protein, 5 aa, Artificial Sequence
  source: /note=“Description of Artificial Sequence Synthetic peptide”
DHTIH

SEQ ID NO: 22 - protein, 17 aa, Artificial Sequence
  source: /note=“Description of Artificial Sequence Synthetic peptide”
YIYPRSGSINYNEKFKG

SEQ ID NO: 23 - protein, 9 aa, Artificial Sequence
  source: /note=“Description of Artificial Sequence Synthetic peptide”
RNLFLPMEY

SEQ ID NO: 24 - protein, 7 aa, Artificial Sequence
  source: /note=“Description of Artificial Sequence Synthetic peptide”
GYTFTDH

SEQ ID NO: 25 - protein, 6 aa, Artificial Sequence
  source: /note=“Description of Artificial Sequence Synthetic peptide”
YPRSGS

SEQ ID NO: 26 - protein, 9 aa, Artificial Sequence
  source: /note=“Description of Artificial Sequence Synthetic peptide”
RNLFLPMEY

SEQ ID NO: 27 - protein, 118 aa, Artificial Sequence
  source: /note=“Description of Artificial Sequence Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSS

SEQ ID NO: 28 - DNA, 354 bp, Artificial Sequence
  source: /note=“Description of Artificial Sequence Synthetic polynucleotide”
gaggtgcagctggtgcagtcaggcgccgaagtgaagaagcccggcgagtcactgaagattagctgtaaagtctcaggctacaccttcaccgatcacactattcactggatgagacagatgcccggtaaaggcctggagtggatgggctatatctaccctagatcaggctctattaactataacgagaagtttaagggtcaggtcacaattagcgccgataagtctagctctaccgcctacctgcagtggtctagcctgaaggctagtgacaccgctatgtactactgcgctagacgtaacctgttcctgcctatggaatactggggtcagggcaccctggtcaccgtgtctagc

SEQ ID NO: 29 - protein, 448 aa, Artificial Sequence
  source: /note=“Description of Artificial Sequence Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1344 bp, Artificial Sequence
  source: /note=“Description of Artificial Sequence Synthetic polynucleotide”
gaggtgcagctggtgcagtcaggcgccgaagtgaagaagcccggcgagtcactgaagattagctgtaaagtctcaggctacaccttcaccgatcacactattcactggatgagacagatgcccggtaaaggcctggagtggatgggctatatctaccctagatcaggctctattaactataacgagaagtttaagggtcaggtcacaattagcgccgataagtctagctctaccgcctacctgcagtggtctagcctgaaggctagtgacaccgctatgtactactgcgctagacgtaacctgttcctgcctatggaatactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 31 - protein, 17 aa, Artificial Sequence
  source: /note=“Description of Artificial Sequence Synthetic peptide”
RSSQSLLSSGDQKNYLT

SEQ ID NO: 32 - protein, 7 aa, Artificial Sequence
  source: /note=“Description of Artificial Sequence Synthetic peptide”
WASTRES

SEQ ID NO: 33 - protein, 9 aa, Artificial Sequence
  source: /note=“Description of Artificial Sequence Synthetic peptide”
QNDYRYPLT

SEQ ID NO: 34 - protein, 13 aa, Artificial Sequence
  source: /note=“Description of Artificial Sequence Synthetic peptide”
SQSLLSSGDQKNY

SEQ ID NO: 35 - protein, 3 aa, Artificial Sequence
  source: /note=“Description of Artificial Sequence Synthetic peptide”
WAS

SEQ ID NO: 36 - protein, 6 aa, Artificial Sequence
  source: /note=“Description of Artificial Sequence Synthetic peptide”
DYRYPL

SEQ ID NO: 37 - protein, 113 aa, Artificial Sequence
  source: /note=“Description of Artificial Sequence Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIK

SEQ ID NO: 38 - DNA, 339 bp, Artificial Sequence
  source: /note=“Description of Artificial Sequence Synthetic polynucleotide”
gatatcgtgatgactcagacccccctgagcctgcccgtgacccctggcgagcctgcctctattagctgtagatctagtcagtcactgctgtctagcggcgatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagataccccctgaccttcggtcagggcactaagctggagattaag

SEQ ID NO: 39 - protein, 220 aa, Artificial Sequence
  source: /note=“Description of Artificial Sequence Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 660 bp, Artificial Sequence
  source: /note=“Description of Artificial Sequence Synthetic polynucleotide”
gatatcgtgatgactcagacccccctgagcctgcccgtgacccctggcgagcctgcctctattagctgtagatctagtcagtcactgctgtctagcggcgatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagataccccctgaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 41 - protein, 5 aa, Artificial Sequence
  source: /note=“Description of Artificial Sequence Synthetic peptide”
DHTIH

SEQ ID NO: 42 - protein, 17 aa, Artificial Sequence
  source: /note=“Description of Artificial Sequence Synthetic peptide”
YIYPRSGSINYNEKFKG

SEQ ID NO: 43 - protein, 9 aa, Artificial Sequence
  source: /note=“Description of Artificial Sequence Synthetic peptide”
RNLFLPMEY

SEQ ID NO: 44 - protein, 7 aa, Artificial Sequence
  source: /note=“Description of Artificial Sequence Synthetic peptide”
GYTFTDH

SEQ ID NO: 45 - protein, 6 aa, Artificial Sequence
  source: /note=“Description of Artificial Sequence Synthetic peptide”
YPRSGS

SEQ ID NO: 46 - protein, 9 aa, Artificial Sequence
  source: /note=“Description of Artificial Sequence Synthetic peptide”
RNLFLPMEY

SEQ ID NO: 47 - protein, 118 aa, Artificial Sequence; identical to SEQ ID NO: 27
  source: /note=“Description of Artificial Sequence Synthetic polypeptide”

SEQ ID NO: 48 - DNA, 354 bp, Artificial Sequence; identical to SEQ ID NO: 28
  source: /note=“Description of Artificial Sequence Synthetic polynucleotide”

SEQ ID NO: 49 - protein, 448 aa, Artificial Sequence; identical to SEQ ID NO: 29
  source: /note=“Description of Artificial Sequence Synthetic polypeptide”

SEQ ID NO: 50 - DNA, 1344 bp, Artificial Sequence; identical to SEQ ID NO: 30
  source: /note=“Description of Artificial Sequence Synthetic polynucleotide”

SEQ ID NO: 51 - protein, 17 aa, Artificial Sequence
  source: /note=“Description of Artificial Sequence Synthetic peptide”
RSSQSLLSSGNQKNYLT

SEQ ID NO: 52 - protein, 7 aa, Artificial Sequence
  source: /note=“Description of Artificial Sequence Synthetic peptide”
WASTRES

SEQ ID NO: 53 - protein, 9 aa, Artificial Sequence
  source: /note=“Description of Artificial Sequence Synthetic peptide”
QNDYSYPLT

SEQ ID NO: 54 - protein, 13 aa, Artificial Sequence
  source: /note=“Description of Artificial Sequence Synthetic peptide”
SQSLLSSGNQKNY

SEQ ID NO: 55 - protein, 3 aa, Artificial Sequence
  source: /note=“Description of Artificial Sequence Synthetic peptide”
WAS

SEQ ID NO: 56 - protein, 6 aa, Artificial Sequence
  source: /note=“Description of Artificial Sequence Synthetic peptide”
DYSYPL

SEQ ID NO: 57 - protein, 113 aa, Artificial Sequence
  source: /note=“Description of Artificial Sequence Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEIK

SEQ ID NO: 58 - DNA, 339 bp, Artificial Sequence
  source: /note=“Description of Artificial Sequence Synthetic polynucleotide”
gatatcgtgatgactcagacccccctgagcctgcccgtgacccctggcgagcctgcctctattagctgtagatctagtcagtcactgctgtctagcggtaatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagctaccccctgaccttcggtcagggcactaagctggagattaag

SEQ ID NO: 59 - protein, 220 aa, Artificial Sequence
  source: /note=“Description of Artificial Sequence Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - DNA, 660 bp, Artificial Sequence
  source: /note=“Description of Artificial Sequence Synthetic polynucleotide”
gatatcgtgatgactcagacccccctgagcctgcccgtgacccctggcgagcctgcctctattagctgtagatctagtcagtcactgctgtctagcggtaatcagaagaactacctgacctggtatctgcagaagcccggtcagtcacctcagctgctgatctactgggcctctactagagaatcaggcgtgcccgataggtttagcggtagcggtagtggcaccgacttcaccctgaagatctctagggtggaagccgaggacgtgggcgtctactactgtcagaacgactatagctaccccctgaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 61 - protein, 5 aa, Artificial Sequence
  source: /note=“Description of Artificial Sequence Synthetic peptide”
DHTLH

SEQ ID NO: 62 - protein, 17 aa, Artificial Sequence
  source: /note=“Description of Artificial Sequence Synthetic peptide”
YIYPRSGSTKYNENFRG

SEQ ID NO: 63 - protein, 9 aa, Artificial Sequence
  source: /note=“Description of Artificial Sequence Synthetic peptide”
RLLFLPLDY

SEQ ID NO: 64 - protein, 7 aa, Artificial Sequence
  source: /note=“Description of Artificial Sequence Synthetic peptide”
GYTFTDH

SEQ ID NO: 65 - protein, 6 aa, Artificial Sequence
  source: /note=“Description of Artificial Sequence Synthetic peptide”
YPRSGS

SEQ ID NO: 66 - protein, 9 aa, Artificial Sequence
  source: /note=“Description of Artificial Sequence Synthetic peptide”
RLLFLPLDY

SEQ ID NO: 67 - protein, 118 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSS

SEQ ID NO: 68 - DNA, 354 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgctagacggctgctgttcctgcccctggactactggggtcagggcaccctggtcaccgtgtctagc

SEQ ID NO: 69 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1344 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgctagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 71 - protein, 17 aa, Artificial Sequence
  source: /note=“Description of Artificial Sequence Synthetic peptide”
RSSQSLLSSGNQKSYLT

SEQ ID NO: 72 - protein, 7 aa, Artificial Sequence
  source: /note=“Description of Artificial Sequence Synthetic peptide”
WASTRES

SEQ ID NO: 73 - protein, 9 aa, Artificial Sequence
  source: /note=“Description of Artificial Sequence Synthetic peptide”
QNDYSYPFT

SEQ ID NO: 74 - protein, 13 aa, Artificial Sequence
  source: /note=“Description of Artificial Sequence Synthetic peptide”
SQSLLSSGNQKSY

SEQ ID NO: 75 - protein, 3 aa, Artificial Sequence
  source: /note=“Description of Artificial Sequence Synthetic peptide”
WAS

SEQ ID NO: 76 - protein, 6 aa, Artificial Sequence
  source: /note=“Description of Artificial Sequence Synthetic peptide”
DYSYPF

SEQ ID NO: 77 - protein, 113 aa, Artificial Sequence
  source: /note=“Description of Artificial Sequence Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEIK

SEQ ID NO: 78 - DNA, 339 bp, Artificial Sequence
  source: /note=“Description of Artificial Sequence Synthetic polynucleotide”
gagatcgtgatgactcagtcacccgctaccctgagcctgagccctggcgagagagctacactgagctgtagatctagtcagtcactgctgtctagcggtaatcagaagtcctacctgacctggtatcagcagaagcccggtcaggcccctagactgctgatctactgggcctctactagagagtcagggatccccgctaggtttagcggtagcggtagtggcaccgacttcaccctgactatctctagcctgcagcccgaggacttcgccgtctactactgtcagaacgactatagctaccccttcaccttcggtcagggcactaagctggagattaag

SEQ ID NO: 79 - protein, 220 aa, Artificial Sequence
  source: /note=“Description of Artificial Sequence Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - DNA, 660 bp, Artificial Sequence
  source: /note=“Description of Artificial Sequence Synthetic polynucleotide”
gagatcgtgatgactcagtcacccgctaccctgagcctgagccctggcgagagagctacactgagctgtagatctagtcagtcactgctgtctagcggtaatcagaagtcctacctgacctggtatcagcagaagcccggtcaggcccctagactgctgatctactgggcctctactagagagtcagggatccccgctaggtttagcggtagcggtagtggcaccgacttcaccctgactatctctagcctgcagcccgaggacttcgccgtctactactgtcagaacgactatagctaccccttcaccttcggtcagggcactaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 81 - protein, 5 aa, Artificial Sequence
  source: /note=“Description of Artificial Sequence Synthetic peptide”
DHTLH

SEQ ID NO: 82 - protein, 17 aa, Artificial Sequence
  source: /note=“Description of Artificial Sequence Synthetic peptide”
YIYPRSGSTKYNENFRG

SEQ ID NO: 83 - protein, 9 aa, Artificial Sequence
  source: /note=“Description of Artificial Sequence Synthetic peptide”
RLLFLPLDY

SEQ ID NO: 84 - protein, 7 aa, Artificial Sequence
  source: /note=“Description of Artificial Sequence Synthetic peptide”
GYTFTDH

SEQ ID NO: 85 - protein, 6 aa, Artificial Sequence
  source: /note=“Description of Artificial Sequence Synthetic peptide”
YPRSGS

SEQ ID NO: 86 - protein, 9 aa, Artificial Sequence
  source: /note=“Description of Artificial Sequence Synthetic peptide”
RLLFLPLDY

SEQ ID NO: 87 - protein, 118 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSS

SEQ ID NO: 88 - DNA, 354 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagc

SEQ ID NO: 89 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - DNA, 1344 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaggggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 91 - protein, 17 aa, Artificial Sequence
  source: /note=“Description of Artificial Sequence Synthetic peptide”
RSSQSLLSSGNQKSYLT

SEQ ID NO: 92 - protein, 7 aa, Artificial Sequence
  source: /note=“Description of Artificial Sequence Synthetic peptide”
WASTRES

SEQ ID NO: 93 - protein, 9 aa, Artificial Sequence
  source: /note=“Description of Artificial Sequence Synthetic peptide”
QNDYSYPFT

SEQ ID NO: 94 - protein, 13 aa, Artificial Sequence
  source: /note=“Description of Artificial Sequence Synthetic peptide”
SQSLLSSGNQKSY

SEQ ID NO: 95 - protein, 3 aa, Artificial Sequence
  source: /note=“Description of Artificial Sequence Synthetic peptide”
WAS

SEQ ID NO: 96 - protein, 6 aa, Artificial Sequence
  source: /note=“Description of Artificial Sequence Synthetic peptide”
DYSYPF

SEQ ID NO: 97 - protein, 113 aa, Artificial Sequence; identical to SEQ ID NO: 77
  source: /note=“Description of Artificial Sequence Synthetic polypeptide”

SEQ ID NO: 98 - DNA, 339 bp, Artificial Sequence; identical to SEQ ID NO: 78
  source: /note=“Description of Artificial Sequence Synthetic polynucleotide”

SEQ ID NO: 99 - protein, 220 aa, Artificial Sequence; identical to SEQ ID NO: 79
  source: /note=“Description of Artificial Sequence Synthetic polypeptide”

SEQ ID NO: 100 - DNA, 660 bp, Artificial Sequence; identical to SEQ ID NO: 80
  source: /note=“Description of Artificial Sequence Synthetic polynucleotide”

SEQ ID NO: 101 - protein, 5 aa, Artificial Sequence
  source: /note=“Description of Artificial Sequence Synthetic peptide”
DHTLH

SEQ ID NO: 102 - protein, 17 aa, Artificial Sequence
  source: /note=“Description of Artificial Sequence Synthetic peptide”
YIYPRSGSTKYNENFKG

SEQ ID NO: 103 - protein, 9 aa, Artificial Sequence
  source: /note=“Description of Artificial Sequence Synthetic peptide”
RLLFLPLDY

SEQ ID NO: 104 - protein, 7 aa, Artificial Sequence
  source: /note=“Description of Artificial Sequence Synthetic peptide”
GYTFTDH

SEQ ID NO: 105 - protein, 6 aa, Artificial Sequence
  source: /note=“Description of Artificial Sequence Synthetic peptide”
YPRSGS

SEQ ID NO: 106 - protein, 9 aa, Artificial Sequence
  source: /note=“Description of Artificial Sequence Synthetic peptide”
RLLFLPLDY

SEQ ID NO: 107 - protein, 118 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSS

SEQ ID NO: 108 - DNA, 354 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaagggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagc

SEQ ID NO: 109 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 1344 bp, Artificial Sequence
  source: /note=“Description of Artificial Sequence Synthetic polynucleotide”
cagattcagctggtgcagtcaggcgccgaagtgaagaaacccggctctagcgtgaaagtcagctgtaaagtctcaggctacaccttcaccgatcacaccctgcactggatgagacaggccccaggtcagggcctggagtggatgggctatatctaccctagatcaggctctactaagtataacgagaactttaagggtagagtgactatcaccgccgacactagctctagcaccgcctatatggaactgtctagcctgagatcagaggacaccgccgtctactactgcgtcagacggctgctgttcctgcccctggactactggggtcagggcaccctggtcaccgtgtctagcgctagcactaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 111 - protein, 17 aa, Artificial Sequence
  source: /note=“Description of Artificial Sequence Synthetic peptide”
RSSQSLLSSGNQKSYLT

SEQ ID NO: 112 - protein, 7 aa, Artificial Sequence
  source: /note=“Description of Artificial Sequence Synthetic peptide”
WASTRES

SEQ ID NO: 113 - protein, 9 aa, Artificial Sequence
  source: /note=“Description of Artificial Sequence Synthetic peptide”
QNDYSYPFT

SEQ ID NO: 114 - protein, 13 aa, Artificial Sequence
  source: /note=“Description of Artificial Sequence Synthetic peptide”
SQSLLSSGNQKSY

SEQ ID NO: 115 - protein, 3 aa, Artificial Sequence
  source: /note=“Description of Artificial Sequence Synthetic peptide”
WAS

SEQ ID NO: 116 - protein, 6 aa, Artificial Sequence
  source: /note=“Description of Artificial Sequence Synthetic peptide”
DYSYPF

SEQ ID NO: 117 - protein, 113 aa, Artificial Sequence; identical to SEQ ID NO: 77
  source: /note=“Description of Artificial Sequence Synthetic polypeptide”

SEQ ID NO: 118 - DNA, 339 bp, Artificial Sequence; identical to SEQ ID NO: 78
  source: /note=“Description of Artificial Sequence Synthetic polynucleotide”

SEQ ID NO: 119 - protein, 220 aa, Artificial Sequence; identical to SEQ ID NO: 79
  source: /note=“Description of Artificial Sequence Synthetic polypeptide”

SEQ ID NO: 120 - DNA, 660 bp, Artificial Sequence; identical to SEQ ID NO: 80
  source: /note=“Description of Artificial Sequence Synthetic polynucleotide”

SEQ ID NO: 121 - protein, 829 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 122 - protein, 822 aa, Rattus norvegicus
MELLSGPLVFLLLLLQVCWLRSVVSESYREDFIREAGVTLEVGGTDLEPSQALEKEALAGHEVLGADSGGIITLNRETVQGGKGVMNSPPSRILRRRKREWVMPPISVPENGKGPFPQRLNQLKSNKDRGTKLFYSITGPGADSPPEGVFTIEKETGWLLLNMPLDREKIVKYELFGHAVSENGASVEEPMNISIIVTDQNDNKPKFTQDTFRGSVPEGVMPGTSVMQVTATDEDDAINTYNGVVAYSIHSQEPKEPHDLMFTIHKSTGTISVISSGLDREKVPEYTLTIQATDMDGDGSTTTAEAVVQILDTNDNAPEFQPQKYEAWVPENAVGHEVQRLTVTDLDSPNSPAWRATYHIVGGDDGDHFTVATHPETNQGILTTKKGLDFEAQNQHTLYIEVTNEASFAVKLPTATATVVVHVEDVNEAPVFVPPSKVIEAQEGISIGELVCVYTAQDPDKEEQKISYSILRDPASWLAVDPDSGQITAAGILDREDEQFVKNDIYEVMVLATDNGNPPTTGTGTLLLTLTDINDHGPIPEPRQIIICNQSPVPQVLNITDKDLSPNSSPFQAQLTHDSDIYWMAEVSEKGDTVALSLKKFLKQDTYDLHLSLSDHGNREQLTMIRATVCDCHGQVLNDCPRPWKGGFILPVLGAVLALLTLLLALLLLVRKKRKVKEPLLLPEDDTRDNVFYYGEEGGGEEDQDYDITQLHRGLEARPEVVLRNDVAPTFIPTPMYRPRPANPDEIGNFIIENLKAANTDPTAPPYDSLLVFDYEGSGSDAASLSSLTSSTSDQDQDYNYLTEWGSRFKKLADMYGGGEDD

SEQ ID NO: 123 - protein, 822 aa, Mus musculus
MELLSGPHAFLLLLLQVCWLRSVVSEPYRAGFIGEAGVTLEVEGTDLEPSQVLGKVALAGQGMHHADNGDIIMLTRGTVQGGKDAMHSPPTRILRRRKREWVMPPIFVPENGKGPFPQRLNQLKSNKDRGTKIFYSITGPGADSPPEGVFTIEKESGWLLLHMPLDREKIVKYELYGHAVSENGASVEEPMNISIIVTDQNDNKPKFTQDTFRGSVLEGVMPGTSVMQVTATDEDDAVNTYNGVVAYSIHSQEPKEPHDLMFTIHKSTGTISVISSGLDREKVPEYRLTVQATDMDGEGSTTTAEAVVQILDANDNAPEFEPQKYEAWVPENEVGHEVQRLTVTDLDVPNSPAWRATYHIVGGDDGDHFTITTHPETNQGVLTTKKGLDFEAQDQHTLYVEVTNEAPFAVKLPTATATVVVHVKDVNEAPVFVPPSKVIEAQEGISIGELVCIYTAQDPDKEDQKISYTISRDPANWLAVDPDSGQITAAGILDREDEQFVKNNVYEVMVLATDSGNPPTTGTGTLLLTLTDINDHGPIPEPRQIIICNQSPVPQVLNITDKDLSPNSSPFQAQLTHDSDIYWMAEVSEKGDTVALSLKKFLKQDTYDLHLSLSDHGNREQLTMIRATVCDCHGQVFNDCPRPWKGGFILPILGAVLALLTLLLALLLLVRKKRKVKEPLLLPEDDTRDNVFYYGEEGGGEEDQDYDITQLHRGLEARPEVVLRNDVVPTFIPTPMYRPRPANPDEIGNFIIENLKAANTDPTAPPYDSLLVFDYEGSGSDAASLSSLTTSASDQDQDYNYLNEWGSRFKKLADMYGGGEDD

SEQ ID NO: 124 - protein, 595 aa, Artificial Sequence
  source: /note=“Description of Artificial Sequence Synthetic polypeptide”
MKFLVNVALVFMVVYISYIYADHQTSLYKKAGFEGDRTDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIHTYNGVVAYSIHSQEPKDPHDLMFTIHRSTGTISVISSGLDREKVPEYTLTIQATDMDGDGSTTTAVAVVEILDANDNAPVFDPQKYESHVPENAVGHEVQRLTVTDLDAPNSPAWRATYLIVGGDDGDHFTIATHPESNQGILTTRKGLDFEAKNQHTLYVEVTNEAPFVLKLPTSTATIVVHVEDVNEAPVFVPPSKVVEVQEGIPTGEAVCVYTAKDPDKENQKISYRILRDPAGWLAMDPDSGQVTVAGTLDREDERFVRNNIYEVMVLAVDNGSPPTTGTGTLLLTLIDVNDHGPVPEPREITICNQSPESQVLNITDKDLSPHTSPFQAQLTDDSDIYWMAEVNEKDDTVVLSLKKFLKQDTYDVHLSLSDHGNKEQLTVIRATVCDCHGHVEKCPDPWKGGGAHHHHHHGA

SEQ ID NO: 125 - protein, 595 aa, Artificial Sequence
  source: /note=“Description of Artificial Sequence Synthetic polypeptide”
MKFLVNVALVFMVVYISYIYADHQTSLYKKAGFEGDRTDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIHTYNGVVAYSIHSQEPKDPHDLMFTIHRSTGTISVISSGLDREKVPEYTLTIQATDMDGDGSTTTAVAVVEILDANDNAPVFDPQKYESHVPENAVGHEVQRLTVTDLDAPNSPAWRATYLIVGGDDGDHFTIATHPESNQGILTTRKGLDFEAKNQHTLYVEVTNEAPFVLKLPTSTATIVVHVEDVNEAPVFVPPSKVVEVQEGIPTGEAVCVYTAKDPDKENQKISYRILRDPAGWLAMDPDSGQVTVAGTLDREDERFVRNNIYEVMVLAVDNGSPPTTGTGTLLLTLIDVNDHGPVPEPREITICNQSPESQVLNITDKDLSPHTSPFQAQLTDDSDIYWMAEVNEKDDTVVLSLKKFLKQGTYDVHLSLSDHGNKEQLTVIRATVCDCHGHVEKCPDPWKGGGAHHHHHHGA

SEQ ID NO: 126 - protein, 829 aa, Homo sapiens; identical to SEQ ID NO: 121

SEQ ID NO: 127 - protein, 219 aa, Artificial Sequence
  source: /note=“Description of Artificial Sequence Synthetic polypeptide”
GPDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDN

SEQ ID NO: 128 - protein, 244 aa, Artificial Sequence
  source: /note=“Description of Artificial Sequence Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSEFDYKDDDDKGAPHHHHHH

SEQ ID NO: 129 - protein, 213 aa, Artificial Sequence
  source: /note=“Description of Artificial Sequence Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 130 - protein, 453 aa, Artificial Sequence
  source: /note=“Description of Artificial Sequence Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 131 - protein, 453 aa, Artificial Sequence
  source: /note=“Description of Artificial Sequence Synthetic polypeptide”
QVQLQQSGPGLVKPSQTLSLTCAISGDSVSSQSAAWNWIRQSPSRGLEWLGRIYYRSKWYNDYALSVKSRITINPDTSKNQFSLQLNSVTPEDTAVYYCARGEGYGREGFAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 213 aa, Artificial Sequence
  source: /note=“Description of Artificial Sequence Synthetic polypeptide”
DIQMTQSPSSLSASVGDRVTITCRASQTISNTLAWYQQKPGKAPKLLIYAASNLQSGVPSRFSGSGSGTDFTLTISSLQPEDFATYYCQQYLSWFTFGQGTKVEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 448 aa, Artificial Sequence
  source: /note=“Description of Artificial Sequence Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 134 - protein, 448 aa, Artificial Sequence
  source: /note=“Description of Artificial Sequence Synthetic polypeptide”
EVQLVQSGAEVKKPGESLKISCKVSGYTFTDHTIHWMRQMPGKGLEWMGYIYPRSGSINYNEKFKGQVTISADKSSSTAYLQWSSLKASDTAMYYCARRNLFLPMEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 220 aa, Artificial Sequence
  source: /note=“Description of Artificial Sequence Synthetic polypeptide”
DIVMTQTPLSLPVTPGEPASISCRSSQSLLSSGDQKNYLTWYLQKPGQSPQLLIYWASTRESGVPDRFSGSGSGTDFTLKISRVEAEDVGVYYCQNDYRYPLTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 448 aa, Artificial Sequence; identical to SEQ ID NO: 133
  source: /note=“Description of Artificial Sequence Synthetic polypeptide”

SEQ ID NO: 137 - protein, 448 aa, Artificial Sequence; identical to SEQ ID NO: 134
  source: /note=“Description of Artificial Sequence Synthetic polypeptide”

SEQ ID NO: 138 - protein, 220 aa, Artificial Sequence
  source: /note=“Description of Artificial Sequence Synthetic polypeptide”
DIVMTQTPLSLPVTPGEPASISCRSSQSLLSSGNQKNYLTWYLQKPGQSPQLLIYWASTRESGVPDRFSGSGSGTDFTLKISRVEAEDVGVYYCQNDYSYPLTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 140 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCARRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 220 aa, Artificial Sequence
  source: /note=“Description of Artificial Sequence Synthetic polypeptide”
EIVMTQSPATLSLSPGERATLSCRSSQSLLSSGNQKSYLTWYQQKPGQAPRLLIYWASTRESGIPARFSGSGSGTDFTLTISSLQPEDFAVYYCQNDYSYPFTFGQGTKLEICRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 143 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFRGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 220 aa, Artificial Sequence; identical to SEQ ID NO: 141
  source: /note=“Description of Artificial Sequence Synthetic polypeptide”

SEQ ID NO: 145 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 146 - protein, 448 aa, Artificial Sequence
  source: /note=“Description of Artificial Sequence Synthetic polypeptide”
QIQLVQSGAEVKKPGSSVKVSCKVSGYTFTDHTLHWMRQAPGQGLEWMGYIYPRSGSTKYNENFKGRVTITADTSSSTAYMELSSLRSEDTAVYYCVRRLLFLPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 220 aa, Artificial Sequence; identical to SEQ ID NO: 141
  source: /note=“Description of Artificial Sequence Synthetic polypeptide”

SEQ ID NO: 148 - protein, 331 aa, Artificial Sequence
  source: /note=“Description of Artificial Sequence Synthetic polypeptide”
SASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 149 - protein, 331 aa, Artificial Sequence
  source: /note=“Description of Artificial Sequence Synthetic polypeptide”
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 108 aa, Artificial Sequence
  source: /note=“Description of Artificial Sequence Synthetic polypeptide”
CRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 369 bp, Artificial Sequence
  source: /note=“Description of Artificial Sequence Synthetic polynucleotide”
caggtgcaattgcagcagagcggtccgggcctggtgaaaccgagccagaccctgagcctgacctgcgcgatttccggagatagcgtgagctctcagtctgctgcttggaactggattcgtcagagcccgagccgtggcctcgagtggctgggccgtatctactaccgtagcaaatggtacaacgactatgccttgagcgtgaaaagccgcattaccattaacccggatacttcgaaaaaccagtttagcctgcaactgaacagcgtgaccccggaagatacggccgtgtattattgcgcgcgtggtgaaggttacggtcgtgaaggtttcgctatctggggccaaggcaccctggtgactgttagctca

SEQ ID NO: 152 - DNA, 1359 bp, Artificial Sequence
  source: /note=“Description of Artificial Sequence Synthetic polynucleotide”
caggtgcaattgcagcagagcggtccgggcctggtgaaaccgagccagaccctgagcctgacctgcgcgatttccggagatagcgtgagctctcagtctgctgcttggaactggattcgtcagagcccgagccgtggcctcgagtggctgggccgtatctactaccgtagcaaatggtacaacgactatgccttgagcgtgaaaagccgcattaccattaacccggatacttcgaaaaaccagtttagcctgcaactgaacagcgtgaccccggaagatacggccgtgtattattgcgcgcgtggtgaaggttacggtcgtgaaggtttcgctatctggggccaaggcaccctggtgactgttagctcagcctctacgaaaggcccaagcgtatttcccctggctccttctagtaaatcaacctcaggtggtacagcagcccttggctgcctggtcaaagactatttcccctgtccggtgaccgtctcatggaactcaggtgctttgacatctggtgtgcatacattcccagctgtgctgcaaagtagtggactgtacagcctttccagcgtggtcacggtgccaagtagctccttgggtactcagacttatatctgcaatgtgaaccacaagccctctaacacgaaggtggacaagcgcgtggagcccaaatcttgcgataagacgcatacttgtcccccatgccctgctcctgagctgttgggaggcccgtcagtgttcttgttccctccgaagcctaaggacactttgatgataagtaggacaccagaggtgacttgcgtggtggttgatgtgtcccatgaagatcccgaggtcaaatttaattggtacgtagatggtgtcgaagttcacaatgctaagactaagccaagggaagagcagtacaacagtacatatagggtagtctccgtgctgacagtcctccaccaggactggttgaacggcaaggaatacaaatgtaaggtgtcaaacaaagctctgcctgctcccattgagaaaacaatctctaaagccaaaggccagccgagagagccccaagtctacactttgcccccgagcagggaggaaatgaccaagaatcaggtgagtctgacgtgcctcgtcaaaggattttatccatgcgatattgcagttgaatgggagagcaatggccagccagagaacaactataaaaccacaccacccgtgctcgactctgatggcagcttcttcctctatagcaagctgacagtcgataaatctcgctggcagcaaggcaatgtgttctcctgctccgtcatgcacgaggctttgcataaccattatactcaaaaatctctgtccctgtcacctggtaaa

SEQ ID NO: 153 - DNA, 318 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagactatttctaacactctggcttggtaccagcagaaaccgggcaaagcgccgaaactattaatctacgctgcttctaacctgcaaagcggcgtgccgagccgctttagcggcagcggatccggcaccgatttcaccctgaccattagctctctgcaaccggaagactttgcgacctattattgccagcagtacctgtcttggttcacctttggccagggcacgaaagttgaaattaaa

SEQ ID NO: 154 - DNA, 639 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagactatttctaacactctggcttggtaccagcagaaaccgggcaaagcgccgaaactattaatctacgctgcttctaacctgcaaagcggcgtgccgagccgctttagcggcagcggatccggcaccgatttcaccctgaccattagctctctgcaaccggaagactttgcgacctattattgccagcagtacctgtcttggttcacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 155 - protein, 6 aa, Artificial Sequence
  source: /note=“Description of Artificial Sequence Synthetic 6xHis tag”
HHHHHH

SEQ ID NO: 156 - protein, 4 aa, Artificial Sequence
  source: /note=“Description of Artificial Sequence Synthetic peptide”
RGDS

SEQ ID NO: 157 - protein, 101 aa, Homo sapiens
DWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQ

SEQ ID NO: 158 - protein, 101 aa, Macaca fascicularis; identical to SEQ ID NO: 157

SEQ ID NO: 159 - protein, 59 aa, Homo sapiens
CPENEKGPFPKNLVQIKSNKDKEGKVFYSITGQGADTPPVGVFIIERETGWLKVTEPLD

SEQ ID NO: 160 - protein, 59 aa, Homo sapiens
LPENSRGPFPQELVRIRSDRDKNLSLRYSVTGPGADQPPTGIFIINPISGQLSVTKPLD

SEQ ID NO: 161 - protein, 59 aa, Homo sapiens
VPENGKGPFPQRLNQLKSNKDRDTKIFYSITGPGADSPPEGVFAVEKETGWLLLNKPLD

SEQ ID NO: 162 - protein, 59 aa, Homo sapiens
VPENSRGPFPQQLVRIRSDKDNDIPIRYSITGVGADQPPMEVFSIDSMSGRMYVTRPMD

SEQ ID NO: 163 - protein, 54 aa, Homo sapiens
IDEEKNTSLPHHVGKIKSSVSRKNAKYLLKGEYVGKVFRVDAETGDVFAIERLD

SEQ ID NO: 164 - protein, 56 aa, Homo sapiens
LLEEYTGSDYQYVGKLHSDQDRGDGSLKYILSGDGAGDLFIINENTGDIQATKRLD

SEQ ID NO: 165 - protein, 56 aa, Homo sapiens
VLEEYMGSDPLYVGKLHSDVDKGDGSIKYILSGEGASSIFIIDENTGDIHATKRLD

SEQ ID NO: 166 - protein, 56 aa, Homo sapiens
VLEEFSGPEPILVGRLHTDLDPGSKKIKYILSGDGAGTIFQINDVTGDIHAIKRLD

SEQ ID NO: 167 - protein, 56 aa, Homo sapiens
LLEEYTGTDTQYVGKLHTDQDKGDGNLKYILTGDGAGSLFVIDENTGDIHAAKKLD

SEQ ID NO: 168 - protein, 22 aa, Artificial Sequence
  source: /note=“Description of Artificial Sequence Synthetic peptide”
YGRKKRRQRRRLYRSPAMPENL